(19) United States
(12) Patent Application Publication
(10) Pub. No.: US 2016/0045528 A1
(43) Pub. Date: Feb. 18, 2016

(54) COMBINATION THERAPY FOR TREATING A PARAMYXOVIRUS

(71) Applicant: ALIOS BIOPHARMA, INC., South San Francisco, CA (US)

(72) Inventors: Lawrence M. Blatt, Healdsburg, CA (US); Leonid Beigelman, San Mateo, CA (US); David Bernard Smith, San Mateo, CA (US); Guangyi Wang, Carlsbad, CA (US)

(21) Appl. No.: 14/816,843

(22) Filed: Aug. 3, 2015

Related U.S. Application Data
(60) Provisional application No. 62/182,913, filed on Jun. 22, 2015.
(60) Provisional application No. 62/060,445, filed on Oct. 6, 2014.
(60) Provisional application No. 62/033,551, filed on Aug. 5, 2014.

(51) Int. Cl. A61K 31/7068; A61K 31/4436; A61K 38/21; A61K 31/437; A61K 31/7056; C12N 7/00; A61K 39/42

(52) U.S. Cl. CPC A61K 31/7068; C12N 7/00; A61K 31/4436; A61K 39/42; A61K 31/437; A61K 31/7056; A61K 38/212; C12N 2760/18563

(57) ABSTRACT

Disclosed herein are a combination of compounds and methods of using the combination of compounds for ameliorating, treating and/or preventing a paramyxovirus viral infection.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6.

### REFERENCE TO SEQUENCE LISTING

The present application is filed with a Sequence Listing in Electronic format. The Sequence Listing is provided as a file entitled ALIOS086.txt, created Aug. 3, 2015, which is approximately 4 kb in size. The information in the electronic format of the sequence listing is incorporated herein by reference in its entirety.

### BACKGROUND

1. Field

The present application relates to the fields of chemistry, biochemistry and medicine. More particularly, disclosed herein are a combination of compounds that can be used to ameliorate, treat and/or prevent a paramyxovirus viral.

2. Description

Respiratory viral infections, including upper and lower respiratory tract viral infections, infects and is the leading cause of death of millions of people each year. Upper respiratory tract viral infections involve the nose, sinuses, pharynx and/or larynx. Lower respiratory tract viral infections involve the respiratory system below the vocal cords, including the trachea, primary bronchi and lungs.

Nucleoside analogs are a class of compounds that have been shown to exert antiviral activity both in vitro and in vivo, and thus, have been the subject of widespread research for the treatment of viral infections. Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments disclosed herein relate to a method for ameliorating or treating a paramyxovirus virus infection that can include administering to a subject infected with the paramyxovirus virus an effective amount of a combination of one or more of Compound (A) and one or more of Compound (B), or a pharmaceutical acceptable salt of any of the foregoing, wherein the paramyxovirus virus infection can be selected from a respiratory syncytial virus infection, a parainfluenza virus infection and a metapneumovirus infection.

Other embodiments disclosed herein relate to a method for ameliorating or treating a paramyxovirus virus infection comprising contacting a cell infected with the paramyxovirus virus with an effective amount of a combination of one or more of Compound (A) and one or more of Compound (B), or a pharmaceutical acceptable salt of any of the foregoing, wherein the paramyxovirus virus infection can be selected from a respiratory syncytial virus infection, a parainfluenza virus infection and a metapneumovirus infection.

Still other embodiments disclosed herein relate to use of an effective amount of a combination of one or more of Compound (A) and one or more of Compound (B), or a pharmaceutical acceptable salt of any of the foregoing, for ameliorating or treating a paramyxovirus virus infection, wherein the paramyxovirus virus infection can be selected from a respiratory syncytial virus infection, a parainfluenza virus infection and a metapneumovirus infection

Yet still other embodiments disclosed herein relate to use of an effective amount of a combination of one or more of Compound (A) and one or more of Compound (B), or a pharmaceutical acceptable salt of any of the foregoing, for ameliorating or treating a paramyxovirus virus infection, wherein the paramyxovirus virus infection can be selected from a respiratory syncytial virus infection, a parainfluenza virus infection and a metapneumovirus infection.

### BRIEF DESCRIPTION OF THE DRAWINGS

FIG. 1 shows example anti-RSV agents.

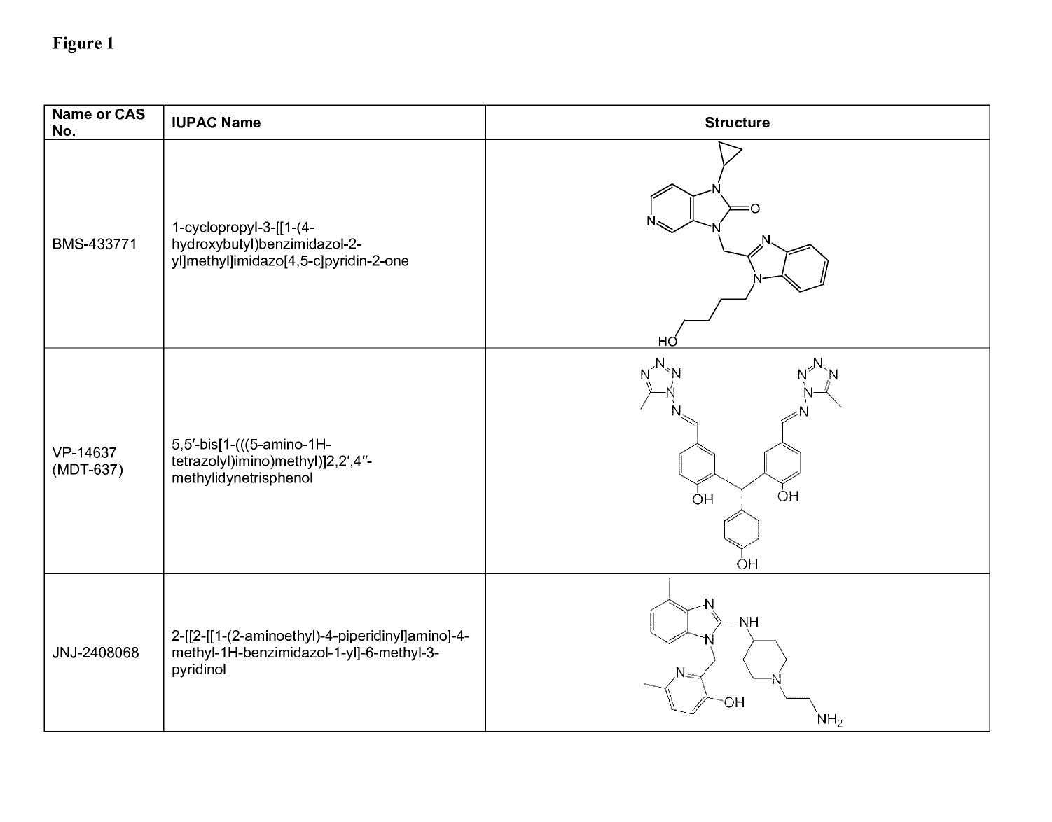

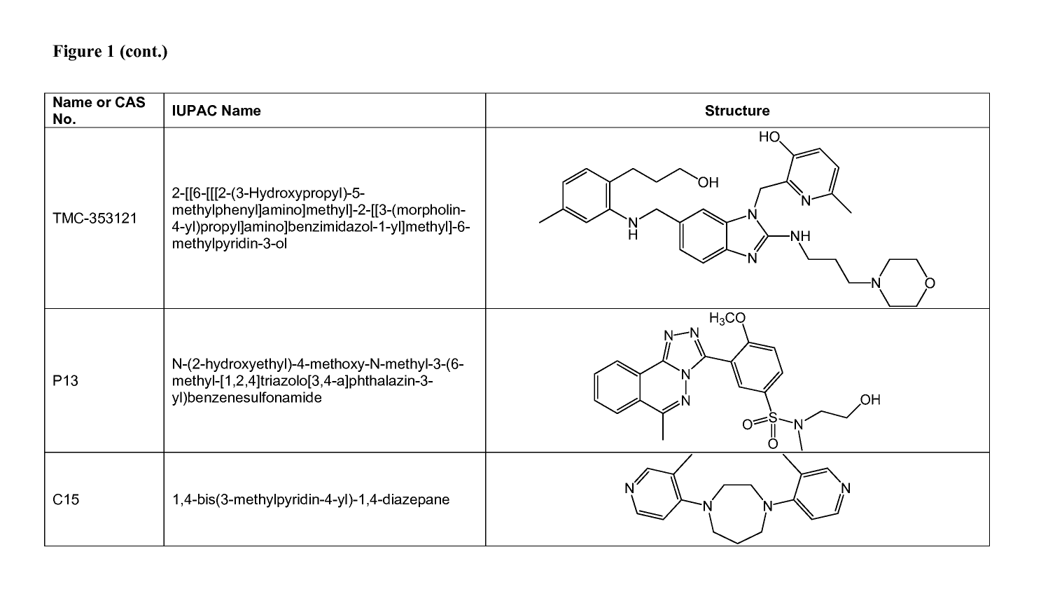

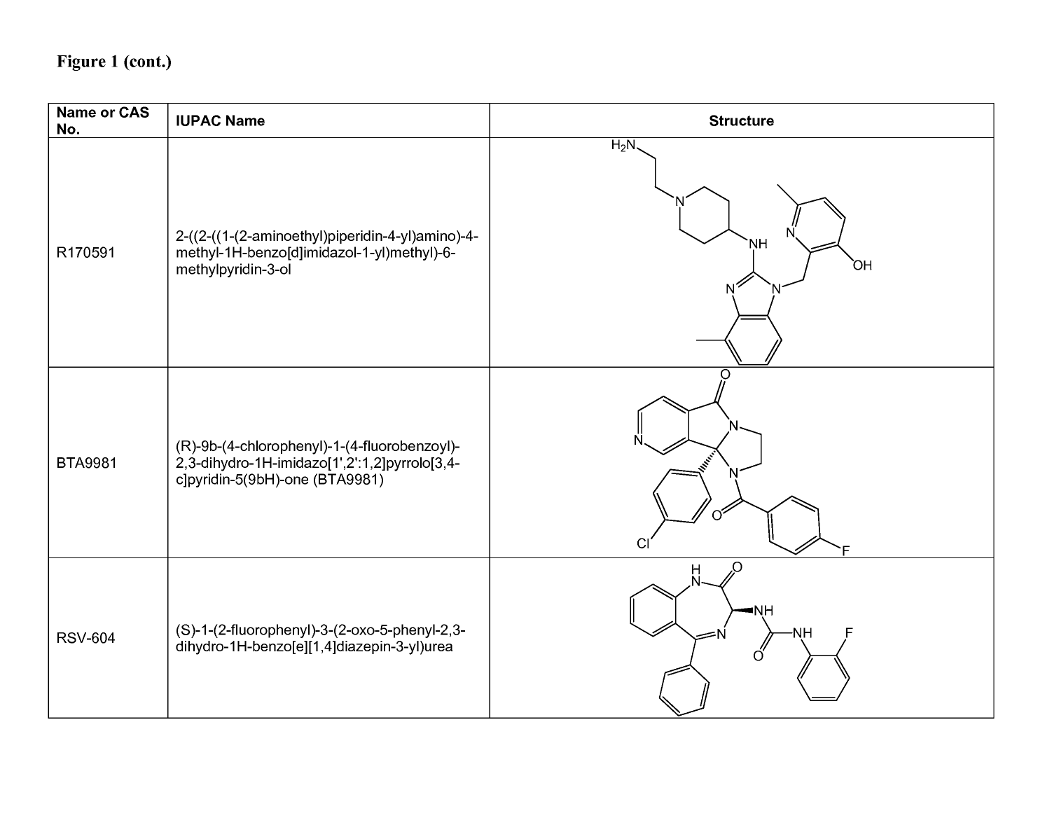

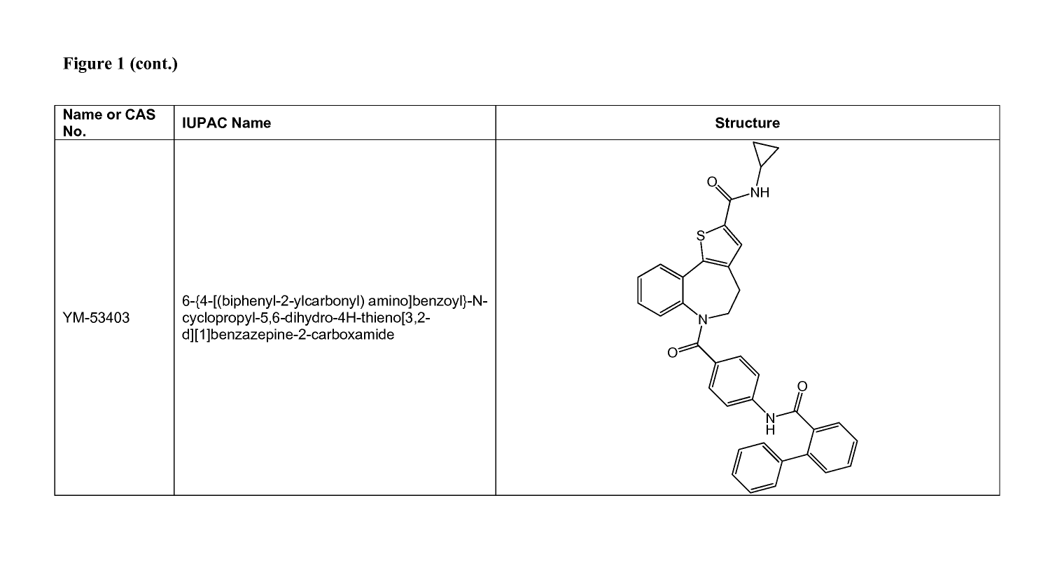

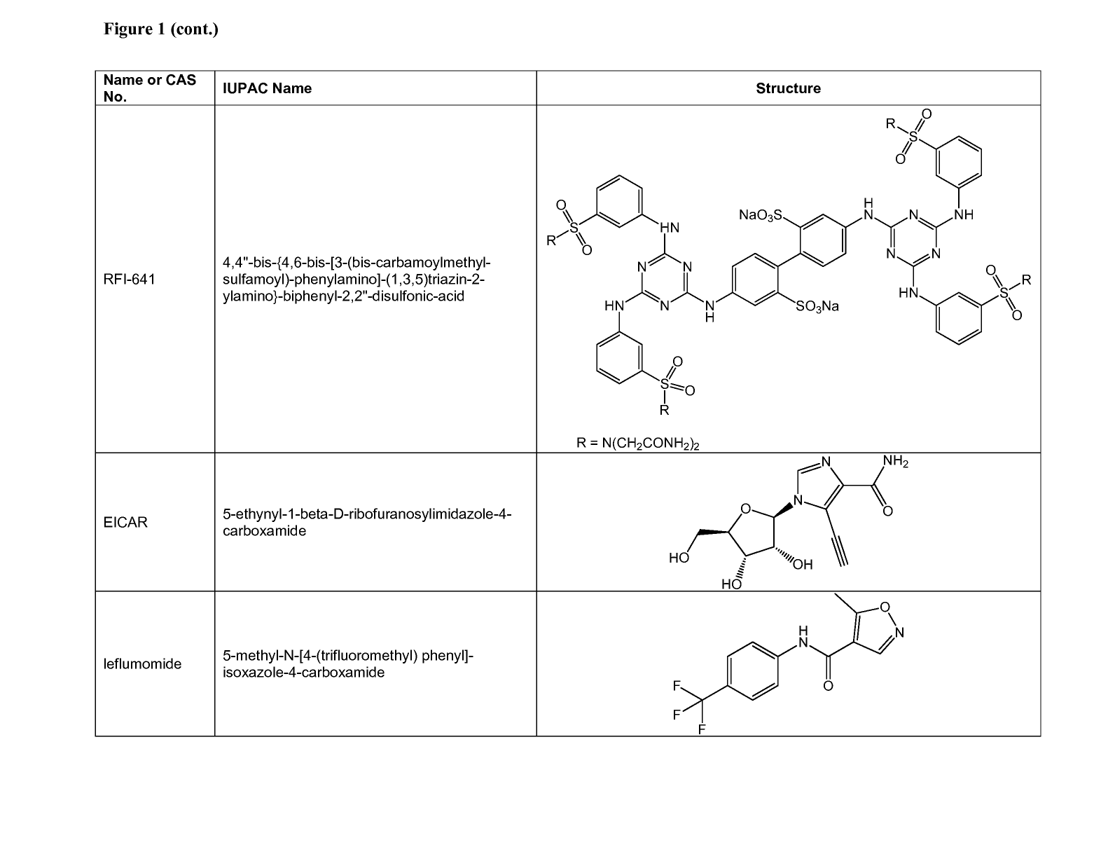

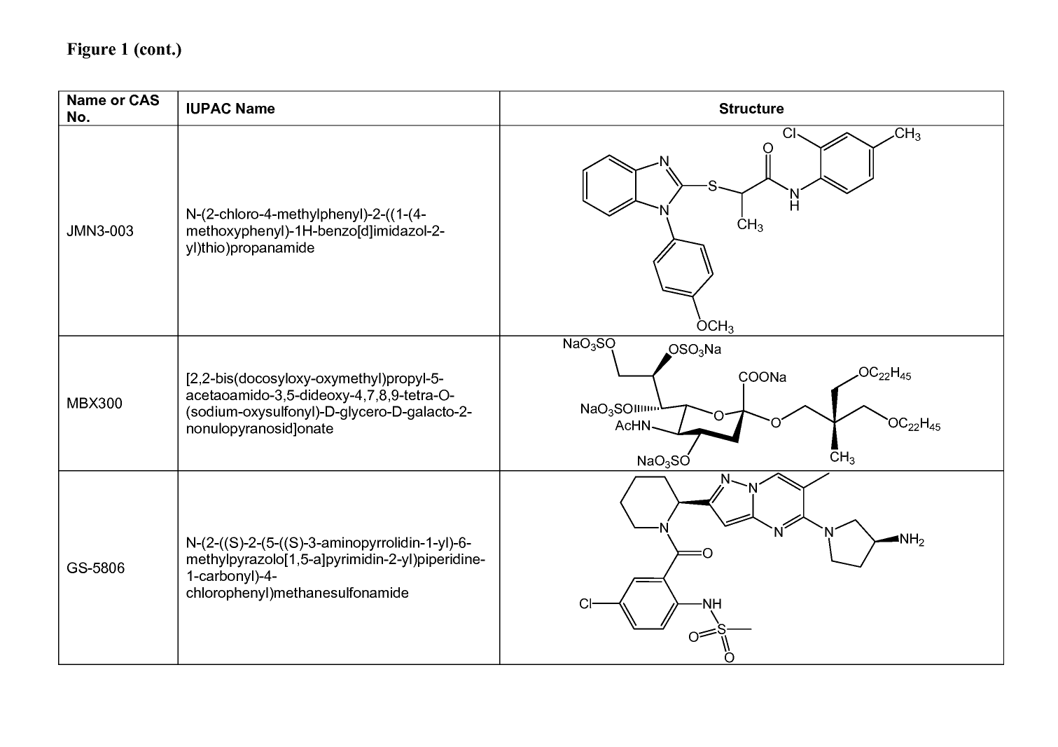

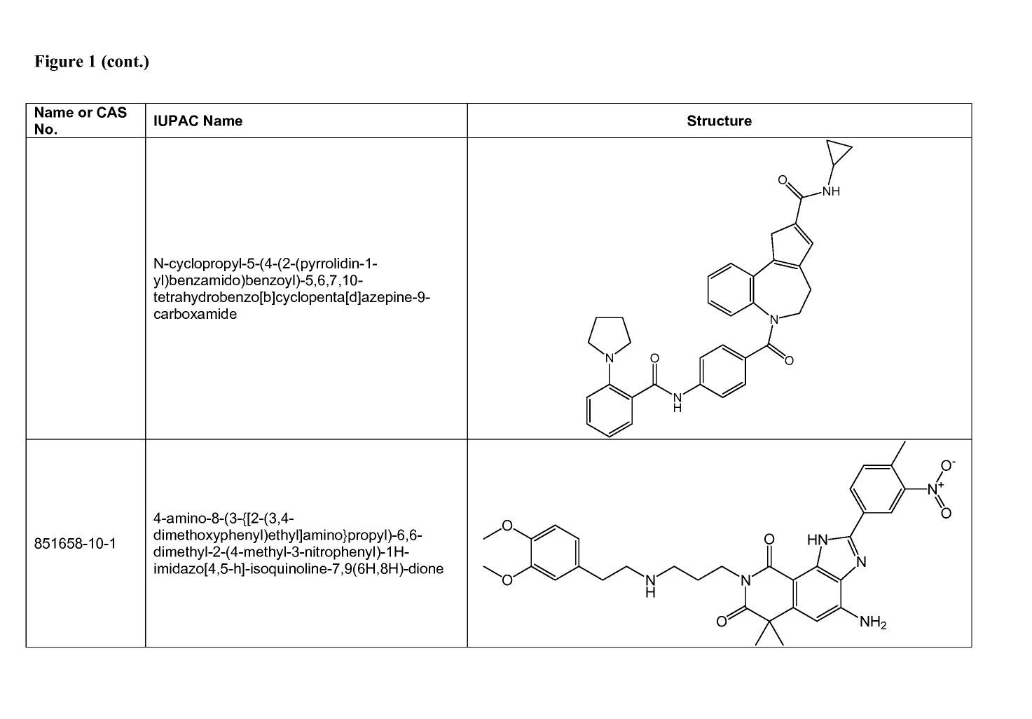

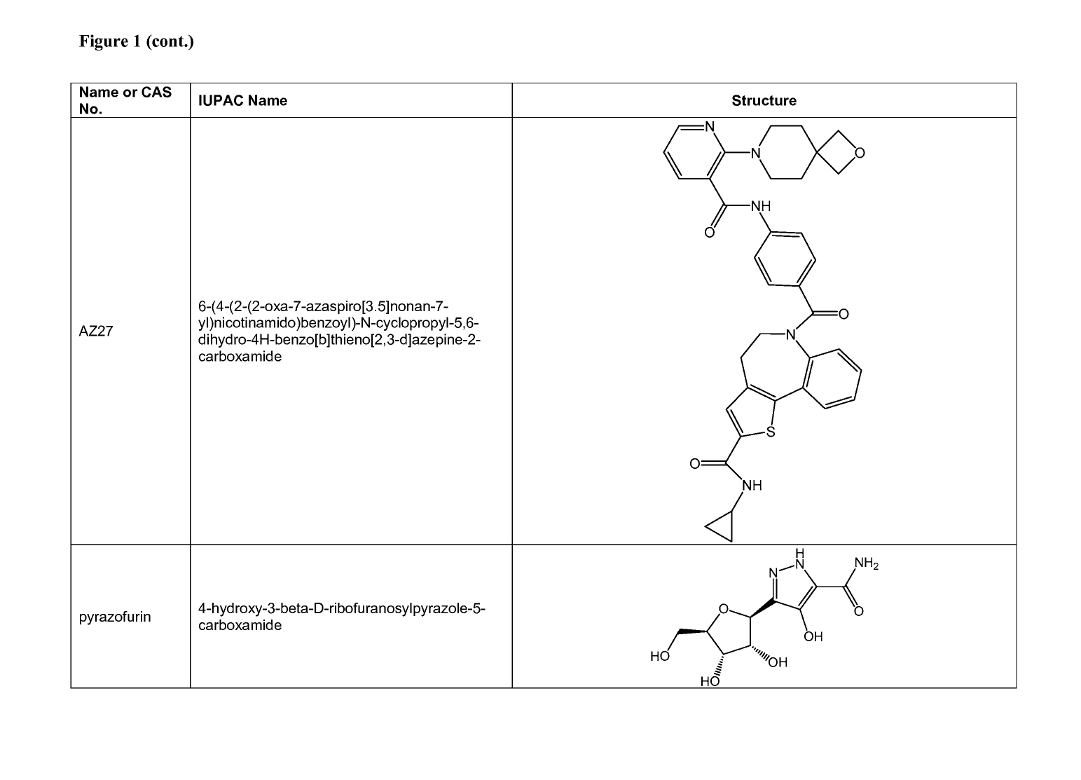

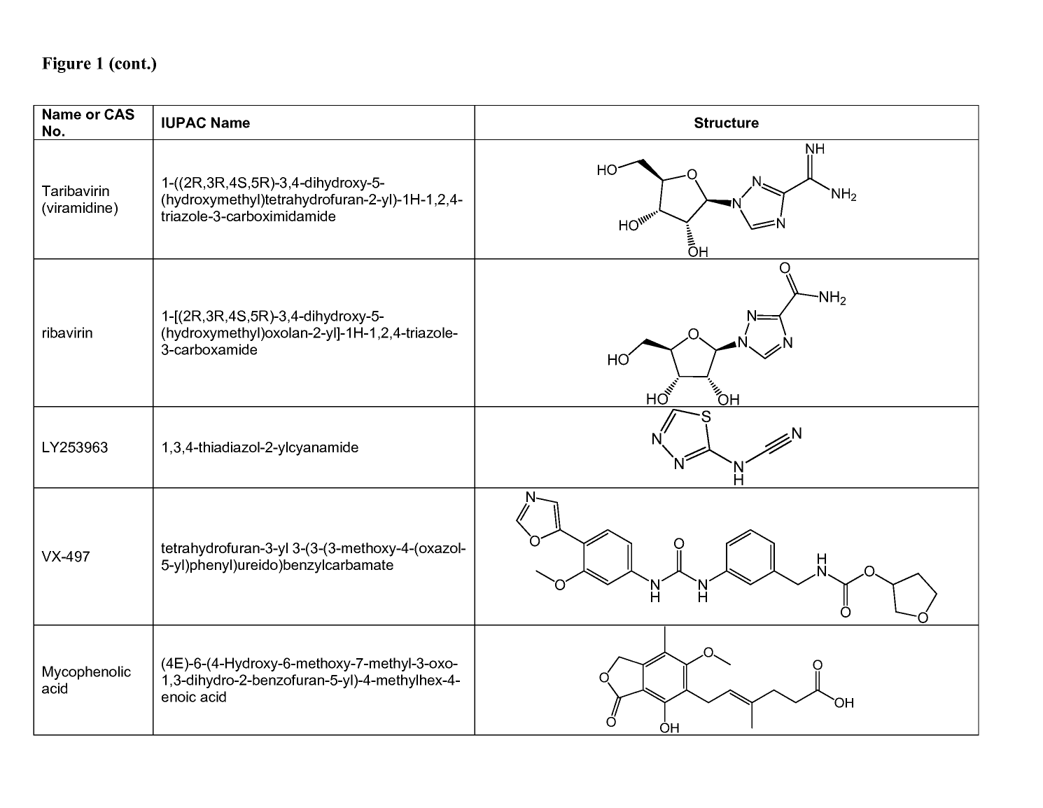

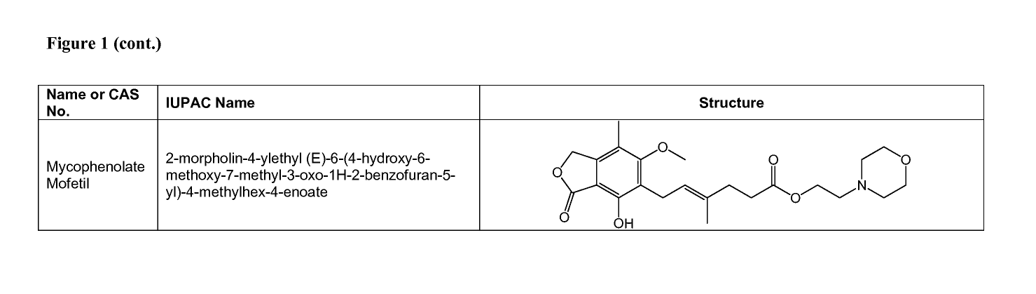

### DETAILED DESCRIPTION

Paramyxoviridae family is a family of single stranded RNA viruses. Several genera of the paramyxoviridae family include respirovirus, rubulavirus, pneumovirus and metapneumovirus. These viruses can be transmitted person to person via direct or close contact with contaminated respiratory droplets or fomites.

Human Respiratory Syncytial Virus (RSV) is a species of pneumovirus and a negative single-stranded RNA virus. RSV can cause respiratory infections, and can be associated with bronchiolitis and pneumonia. Symptoms of an RSV infection include coughing, sneezing, runny nose, fever, decrease in appetite, sore throat, headache and wheezing. RSV is the most common cause of bronchiolitis and pneumonia in children under one year of age in the world, and can be the cause of tracheobronchitis in older children and adults. In the United States, between 75,000 and 125,000 infants are hospitalized each year with RSV. Among adults older than 65 years of age, an estimated 14,000 deaths and 177,000 hospitalizations have been attributed to RSV.

Treatment options for people infected with RSV are currently limited. Antibiotics, usually prescribed to treat bacterial infections, and over-the-counter medication are not effective in treating RSV and may help only to relieve some of the symptoms. In severe cases, a nebulized bronchodilator, such as albuterol, may be prescribed to relieve some of the symptoms, such as wheezing. RespiGam® (RSV-IGIV, Medlmmune, approved for high risk children younger than 24 months of age) and Synagis® (palivizumab, Medlmmune, approved for high risk children younger than 24 months of age) have been approved for prophylactic use against RSV, and Virzole® (ribavirin by aerosol, ICN pharmaceuticals) have been approved for the treatment of RSV.

Parainfluenza viruses are typically negative-sense RNA viruses. Species of respirovirus include human parainfluenza viruses 1 and 3; and species of rubulavirus include human parainfluenza viruses 2 and 4. Human parainfluenza virus includes four serotypes types (HPIV-1, HPIV-2, HPIV-3 and HPIV-4), and human parainfluenza virus 4 (HPIV-4) include two antigenic subgroups, A and B. Human parainfluenza viruses can cause upper and lower respiratory tract infections. Human parainfluenza virus 1 (HPIV-1) and human parainfluenza virus 2 (HPIV-2) can be associated with croup; human parainfluenza virus 3 (HPIV-3) can be associated with bronchiolitis and pneumonia. According to the Centers of Disease Control and Prevention (CDC), there are no vaccines against human parainfluenza viruses.

A species of metapneumovirus is human metapneumovirus. Human metapneumovirus is a negative single-stranded RNA virus. Human metapneumovirus can cause respiratory tract infections, such as upper and lower respiratory tract infections in human, for example young children.

Respiratory infections include colds, croup, pneumonia, bronchitis, tracheobronchitis and bronchiolitis. Symptoms can include a cough, runny nose, nasal congestion, sore throat, fever, difficulty breathing, abnormally rapid breathing, wheezing vomiting, diarrhea and ear infections.

### DEFINITIONS

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, R^{14A}, R^{15A}, R^{16A}, R^{17A}, R^{18A}, R^{19A}, R^{20A}, R^{21A}, R^{22A}, R^{23A}, R^{24A}, R^{25A}, R^{26A}, R^{27A}, R^{28A}, R^{29A}, R^{30A}, R^{31A}, R^{32A}, R^{33A}, R^{34A}, R^{35A}, R^{36A}, R^{37A} and R^{38A} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

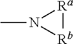

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heterocyclyl, aryl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, sulfenyl, sulfinyl, sulfonyl, haloalkyl, haloalkoxy, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heteroalicyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl, and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. Cycloalkenyl groups can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates.

When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenyl(alkyl), 3-phenyl(alkyl), and naphthyl(alkyl).

As used herein, “heteroaralkyl” and “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

A “(heteroalicyclyl)alkyl” and “(heterocyclyl)alkyl” refer to a heterocyclic or a heteroalicyclylic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂-tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, aralkyl, heteroaryl(alkyl) or heterocyclyl(alkyl) is defined herein. A non-limiting list of alkoxys are methoxy, ethoxy, n-propoxy, 1-methylethoxy(isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an —O-alkyl group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O-” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂-” group wherein each X is a halogen.

A “trihalomethanesulfonamido” group refers to an “X₃CS(O)₂N(R_{A})—” group wherein each X is a halogen, and R_{A} hydrogen, alkyl, alkenyl, alkynyl, cycloalkyl, cycloalkenyl, aryl, heteroaryl, heteroalicyclyl, aralkyl, (heteroaryl)alkyl or (heteroalicyclyl)alkyl.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “—N-linked amino acid” refers to an amino acid that is attached to the indicated moiety via a main-chain amino or mono-substituted amino group. When the amino acid is attached in an —N-linked amino acid, one of the hydrogens that is part of the main-chain amino or mono-substituted amino group is not present and the amino acid is attached via the nitrogen. N-linked amino acids can be substituted or unsubstituted.

The term “—N-linked amino acid ester derivative” refers to an amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include substituted and unsubstituted versions of the following: methyl-O—C(═O)—, ethyl-O—C(═O)—, n-propyl-O—C(═O)—, isopropyl-O—C(═O)—, n-butyl-O—C(═O)—, isobutyl-O—C(═O)—, tert-butyl-O—C(═O)—, neopentyl-O—C(═O)—, cyclopropyl-O—C(═O)—, cyclobutyl-O—C(═O)—, cyclopentyl-O—C(═O)—, cyclohexyl-O—C(═O)—, phenyl-O—C(═O)—, benzyl-O—C(═O)—, and naphthyl-O—C(═O)—. N-linked amino acid ester derivatives can be substituted or unsubstituted.

The term “—O-linked amino acid” refers to an amino acid that is attached to the indicated moiety via the hydroxy from its main-chain carboxylic acid group. When the amino acid is attached in an —O-linked amino acid, the hydrogen that is part of the hydroxy from its main-chain carboxylic acid group is not present and the amino acid is attached via the oxygen. O-linked amino acids can be substituted or unsubstituted.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine.

The term “interferon” is used herein as is commonly understood by one of ordinary skill in the art. Several types of interferons are known to those skilled in the art, such as Type I interferons, Type 2 interferons and Type 3 interferons. A non-limiting list of examples include: alpha-interferons, beta-interferons, delta-interferons, gamma interferons, lambda interferons, omega-interferons, tau-interferons, x-interferons, consensus interferons and asialo-interferons. Interferons can be pegylated. Examples of type 1 interferons include interferon alpha 1A, interferon alpha 1B, interferon alpha 2A, interferon alpha 2B, pegylated-interferon alpha 2a (PEGASYS, Roche), recombinant interferon alpha 2a (ROFERON, Roche), inhaled interferon alpha 2b (AERX, Aradigm), pegylated-interferon alpha 2b (ALBUFERON, Human Genome Sciences/Novartis, PEGINTRON, Schering), recombinant interferon alpha 2b (INTRON A, Schering), pegylated interferon alpha 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical). Examples of type 2 interferons include interferon gamma 1, interferon gamma 2 and pegylated interferon gamma; and examples of type 3 interferons include interferon lambda 1, interferon lambda 2 and interferon lambda 3.

The terms “phosphorothioate” and “phosphothioate” refer to a compound of the general formula

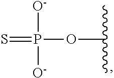

its protonated forms (for example,

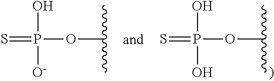 [SMILES: *OP([O-])(O)=S ; *OP(O)(O)=S]

and its tautomers (such as

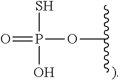 [SMILES: *OP(=O)(O)S]

As used herein, the term “phosphate” is used in its ordinary sense as understood by those skilled in the art, and includes its protonated forms (for example,

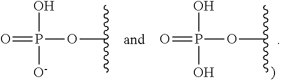 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of a phosphate and a phosphorothioate groups are intended to be included. Examples of tautomers of a phosphorothioate include the following:

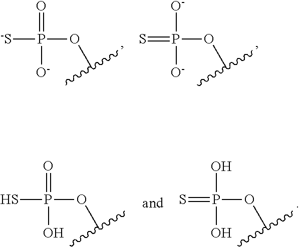

Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### COMPOUNDS

### Compound (A)

Some embodiments described herein relate generally to the use of

Compound (A), or a pharmaceutically acceptable salt thereof, wherein:

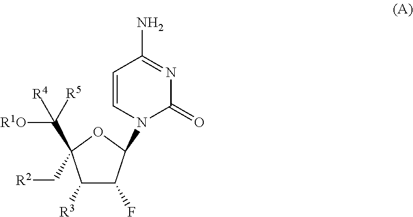

wherein: R¹ can be selected from H (hydrogen), an optionally substituted acyl, an optionally substituted O-linked amino acid,

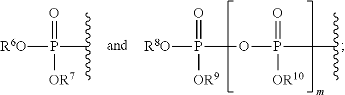

R² can be chloro (Cl) or azido (N₃); R³ can be selected from OH, —OC(═O)R^{A1} and an optionally substituted O-linked amino acid; R⁴ and R⁵ can be independently H (hydrogen) or D (deuterium); R⁶ and R⁷ can be independently absent, H (hydrogen),

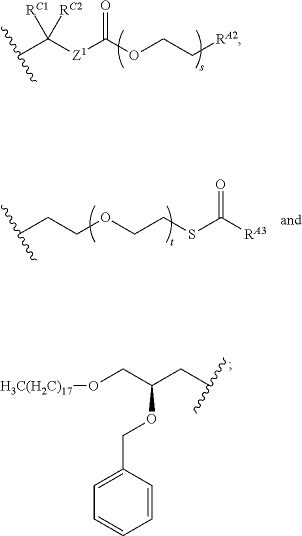

R⁸, R⁹ and each R¹⁰ can be independently absent or H (hydrogen); R^{A1} can be an optionally substituted C₁₋₂₄ alkyl; R^{A2} can be independently selected from H (hydrogen), an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl,

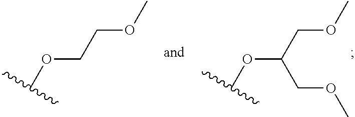

R^{A3} can be selected from H (hydrogen), an optionally substituted C₁₋₂₄ alkyl and an optionally substituted aryl; R^{C1} and R^{C2} can be independently selected from H (hydrogen), an optionally substituted C₁₋₂₄ alkyl and an optionally substituted aryl; m can be 1 or 2; s can be 0, 1, 2 or 3; t can be 0 or 1; and Z¹ can be O (oxygen) or S (sulfur).

In some embodiments, R¹ can be H (hydrogen). When R¹ is H, Compound (A) can be a nucleoside. In other embodiments, R¹ can be an optionally substituted acyl. In other embodiments, R¹ can be —C(═O)R^{B1}, wherein R^{B1} can be selected from an optionally substituted C₁₋₁₂ alkyl, an optionally substituted C₂₋₁₂ alkenyl, an optionally substituted C₂₋₁₂ alkynyl, an optionally substituted C₃₋₈ cycloalkyl, an optionally substituted C₅₋₈ cycloalkenyl, an optionally substituted C₆₋₁₀ aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl). In some embodiments, R^{B1} can be a substituted C₁₋₁₂ alkyl. In other embodiments, R^{B1} can be an unsubstituted C₁₋₁₂ alkyl. In some embodiments, R^{B1} can be an unsubstituted C₁₋₆ alkyl.

In still other embodiments, R¹ can be an optionally substituted O-linked amino acid. Examples of suitable O-linked amino acids include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine. In some embodiments, the O-linked amino acid can have the structure

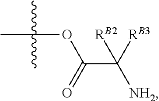

wherein R^{B2} can be selected from hydrogen, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl(C₁₋₆ alkyl); and R^{B3} can be hydrogen or an optionally substituted C₁₋₄-alkyl; or R^{B2} and R^{B3} can be taken together to form an optionally substituted C₃₋₆ cycloalkyl. Those skilled in the art understand that when R¹ is an optionally substituted O-linked amino acid, the oxygen of R¹O— of Compound (A) is part of the optionally substituted O-linked amino acid. For example, when R¹ is

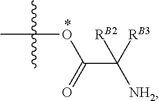

the oxygen indicated with “*” is the oxygen of R¹O— of Compound (A).

When R^{B2} is substituted, R^{B2} can be substituted with one or more substituents selected from N-amido, mercapto, alkylthio, an optionally substituted aryl, hydroxy, an optionally substituted heteroaryl, O-carboxy and amino. In some embodiments, R^{B2} can be an unsubstituted C₁₋₆-alkyl, such as those described herein. In some embodiments, R^{B2} can be hydrogen. In other embodiments, R^{B2} can be methyl. In some embodiments, R^{B3} can be hydrogen. In other embodiments, R^{B3} can be an optionally substituted C₁₋₄-alkyl, such as methyl, ethyl, n-propyl, isopropyl, n-butyl, isobutyl and tert-butyl. In an embodiment, R^{B3} can be methyl. Depending on the groups that are selected for R^{B2} and R^{B3}, the carbon to which R^{B2} and R^{B3} are attached may be a chiral center. In some embodiment, the carbon to which R^{B2} and R^{B3} are attached may be a (R)-chiral center. In other embodiments, the carbon to which R^{B2} and R^{B3} are attached may be a (S)-chiral center.

In yet still other embodiments, R¹ can be

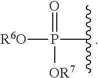

### When R¹ is

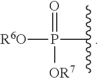

in some embodiments, at least one of R⁶ and R⁷ can be absent or H. In other embodiments, both R⁶ and R⁷ can be independently absent or H. Those skilled in the art understand that when both R⁶ and R⁷ can be independently absent or H, Compound (A) can be a monophosphate. Those skilled in the art also understand that when R⁶ and/or R⁷ are absent, then the oxygen(s) associated with R⁶ and/or R⁷ will have a negative charge. For example, when R⁶ is absent, the oxygen associated with R⁶ will have an associated negative charge.

In some embodiments, at least one of R⁶ and R⁷ can be

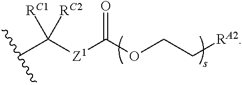

In some embodiments, both R⁶ and R⁷ can be

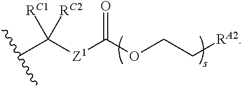

When one or both of R⁶ and R⁷ are

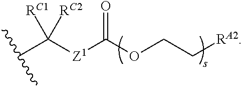

R^{C1} and R^{C2} can be independently selected from hydrogen, an optionally substituted C₁₋₂₄ alkyl and an optionally substituted aryl; R^{A2} can be independently selected from hydrogen, an optionally substituted C₁₋₂₄ alkyl, an optionally substituted aryl, an optionally substituted —O—C₁₋₂₄ alkyl, an optionally substituted —O-aryl, an optionally substituted —O— heteroaryl, an optionally substituted —O-monocyclic heterocyclyl,

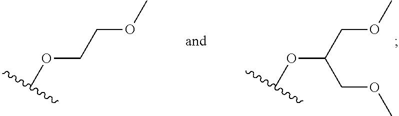

and Z¹ can be independently O (oxygen) or S (sulfur). In some embodiments, R^{C1} and R^{C2} can be hydrogen. In other embodiments, at least one of R^{C1} and R^{C2} can be an optionally substituted C₁₋₂₄ alkyl or an optionally substituted aryl. In some embodiments, R^{A2} can be an optionally substituted C₁₋₂₄ alkyl. In other embodiments, R^{A2} can be an optionally substituted aryl. In still other embodiments, R^{A2} can be an optionally substituted —O—C₁₋₂₄ alkyl or an optionally substituted —O-aryl. In yet still other embodiments, R^{A2} can be an optionally substituted —O-heteroaryl or an optionally substituted —O-monocyclic heterocyclyl. In some embodiments, Z¹ can be O (oxygen). In other embodiments, Z¹ can be S (sulfur). In some embodiments, s can be 0. In other embodiments, s can be 1. In still other embodiments, s can be 2. In yet still other embodiments, s can be 3. In some embodiments, s can be 0, and R^{A2} can be

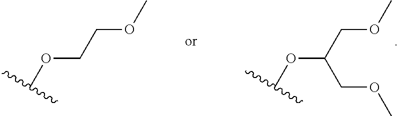

In some embodiments, one or both of R⁶ and R⁷ can be isopropyloxycarbonyloxymethyl (POC). In some embodiments, one or both of R⁶ and R⁷ can be pivaloyloxymethyl (POM). In some embodiments, R⁶ and R⁷ can be both an optionally substituted isopropyloxycarbonyloxymethyl group, and form an optionally substituted bis(isopropyloxycarbonyloxymethyl) (bis(POC)) prodrug. In some embodiments, R⁶ and R⁷ can be both an optionally substituted pivaloyloxymethyl group, and form an optionally substituted bis(pivaloyloxymethyl) (bis(POM)) prodrug.

In some embodiments, R⁶ and R⁷ can be both

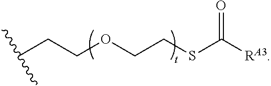

In some embodiments, at least one of R⁶ and R⁷ can be

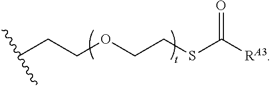

In some embodiments, R^{A3} can be hydrogen. In other embodiments, R^{A3} can be an optionally substituted C₁₋₂₄ alkyl. In still other embodiments, R^{A3} can be an optionally substituted aryl. In some embodiments, R^{A3} can be a C₁₋₆ alkyl, for example, methyl, ethyl, n-propyl, isopropyl, n-butyl, isobutyl, tert-butyl, pentyl (branched and straight-chained) and hexyl (branched and straight-chained). In some embodiments, t can be 0. In other embodiments, t can be 1. In some embodiments, one or both of R⁶ and R⁷ can be an optionally substituted S-acylthioethyl (SATE) and form an optionally substituted SATE ester prodrug.

In some embodiments, one of R⁶ and R⁷ can be

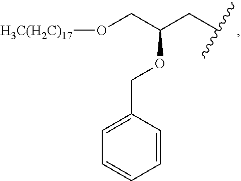

and the other of R⁶ and R⁷ can be absent or H.

In some embodiments, R¹ can be

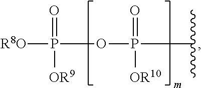

R⁸, R⁹ and each R¹⁰ can be independently absent or hydrogen; and m can be 1 or 2. In some embodiments, m can be 1, and R⁸, R⁹ and R¹⁰ can be independently absent or hydrogen. In other embodiments, m can be 2, and R⁸, R⁹ and each R¹⁰ can be independently absent or hydrogen. Those skilled in the art understand that when m is 1, R¹ can be diphosphate. Likewise, those skilled in the art understand that when m is 2, R¹ can be triphosphate. When R⁸, R⁹ and/or R¹⁰ are absent, those skilled in the art understand that the oxygen associated with R⁸, R⁹ and/or R¹⁰ will have an associated negative charge. For example, when R⁸ is absent, the oxygen associated with R⁸ will have a negative charge, which can be indicated as O⁻.

In some embodiments, R² can be chloro, such that the 2′-position is substituted with a chloromethyl group. In other embodiments, R² can be azido, such that the 2′-position is substituted with an azidomethyl group.

The groups attached to the 3′-position of the ring can vary. In some embodiments, R³ can be OH. In other embodiments, R³ can be —OC(═O)R^{A1}. In some embodiments, R^{A1} can be an optionally substituted C₁₋₆ alkyl. In still other embodiments, R³ can be an optionally substituted O-linked amino acid, such as a O-linked alpha-amino acid. When R³ is an optionally substituted O-linked amino acid, R³ can have the structure

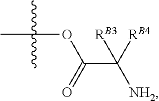

wherein R^{B3} can be selected from hydrogen, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl(C₁₋₆ alkyl); and R^{B4} can be hydrogen or an optionally substituted C₁₋₄-alkyl; or R^{B3} and R^{B4} can be taken together to form an optionally substituted C₃₋₆ cycloalkyl.

When R^{B3} is substituted, R^{B3} can be substituted with one or more substituents selected from N-amido, mercapto, alkylthio, an optionally substituted aryl, hydroxy, an optionally substituted heteroaryl, O-carboxy and amino. In some embodiments, R^{B3} can be an unsubstituted C₁₋₆-alkyl, such as those described herein. In some embodiments, R^{B3} can be hydrogen. In other embodiments, R^{B3} can be methyl. In some embodiments, R^{B4} can be hydrogen. In other embodiments, R^{B4} can be an optionally substituted C₁₋₄-alkyl, such as methyl, ethyl, n-propyl, isopropyl, n-butyl, isobutyl and tert-butyl. In an embodiment, R^{B4} can be methyl. Depending on the groups that are selected for R^{B3} and R^{B4}, the carbon to which R^{B3} and R^{B4} are attached may be a chiral center. In some embodiment, the carbon to which R^{B3} and R^{B4} are attached may be a (R)-chiral center. In other embodiments, the carbon to which R^{B3} and R^{B4} are attached may be a (S)-chiral center.

Examples of suitable

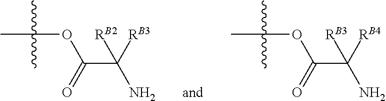

include the following:

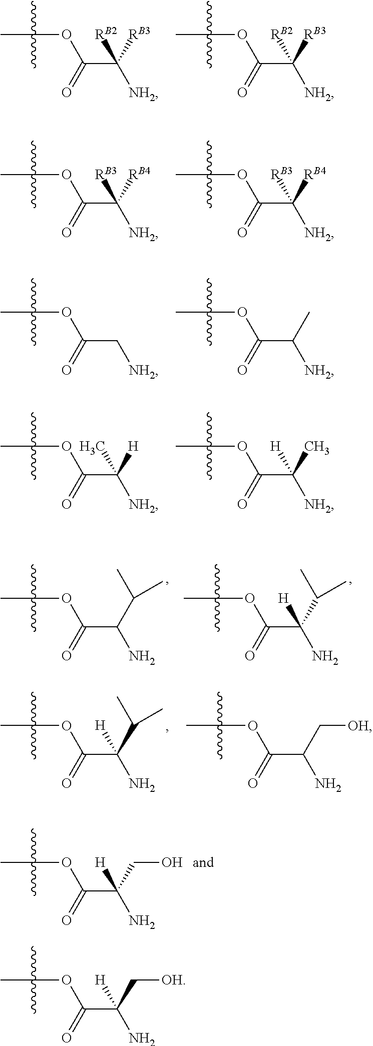

In some embodiments, R⁴ and R⁵ can be both hydrogen (H). In other embodiments, R⁴ and R⁵ can be both deuterium (D). In still other embodiments, one of R⁴ and R⁵ can be hydrogen, and the other of R⁴ and R⁵ can be deuterium.

As described herein, at any position of Compound (A) that has a hydrogen, the hydrogen can be an isotope of hydrogen, such as hydrogen-2 (deuterium). In some embodiments, Compound (A) can be Compound (A1). Some embodiments of Compound (A1) are provided in Table A.

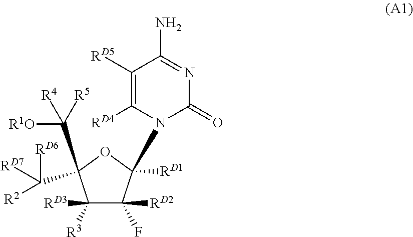

TABLE A

| | R⁴ | | | | | | R^{D6} |
|---|---|---|---|---|---|---|---|
| R² | R⁵ | R^{D1} | R^{D2} | R^{D3} | R^{D4} | R^{D5} | R^{D7} |
| Cl | DD | D | D | D | D | D | DD |
| Cl | DD | D | D | D | D | D | HD |
| Cl | DD | D | D | D | H | D | DD |
| Cl | DD | D | D | D | D | H | DD |
| Cl | DD | D | D | D | D | D | HH |
| Cl | DD | D | D | D | H | D | HD |
| Cl | DD | D | D | D | D | H | HD |
| Cl | DD | D | D | D | H | H | DD |
| Cl | DD | D | D | D | H | D | HH |
| Cl | DD | D | D | D | D | H | HH |
| Cl | DD | D | D | D | H | H | HD |
| Cl | DD | D | D | D | H | H | HH |
| Cl | HD | D | D | D | D | D | DD |
| Cl | HD | D | D | D | D | D | HD |
| Cl | HD | D | D | D | H | D | DD |
| Cl | HD | D | D | D | D | H | DD |
| Cl | HD | D | D | D | D | D | HH |
| Cl | HD | D | D | D | H | D | HD |
| Cl | HD | D | D | D | D | H | HD |
| Cl | HD | D | D | D | H | H | DD |
| Cl | HD | D | D | D | H | D | HH |
| Cl | HD | D | D | D | D | H | HH |
| Cl | HD | D | D | D | H | H | HD |
| Cl | HD | D | D | D | H | H | HH |
| Cl | HH | D | D | D | D | D | DD |
| Cl | HH | D | D | D | D | D | HD |
| Cl | HH | D | D | D | H | D | DD |
| Cl | HH | D | D | D | D | H | DD |
| Cl | HH | D | D | D | D | D | HH |
| Cl | HH | D | D | D | H | D | HD |
| Cl | HH | D | D | D | D | H | HD |
| Cl | HH | D | D | D | H | H | DD |
| Cl | HH | D | D | D | H | D | HH |
| Cl | HH | D | D | D | D | H | HH |
| Cl | HH | D | D | D | H | H | HD |
| Cl | HH | D | D | D | H | H | HH |
| Cl | DD | H | D | D | D | D | DD |
| Cl | DD | H | D | D | D | D | HD |
| Cl | DD | H | D | D | H | D | DD |
| Cl | DD | H | D | D | D | H | DD |
| Cl | DD | H | D | D | D | D | HH |
| Cl | DD | H | D | D | H | D | HD |
| Cl | DD | H | D | D | D | H | HD |
| Cl | DD | H | D | D | H | H | DD |
| Cl | DD | H | D | D | H | D | HH |
| Cl | DD | H | D | D | D | H | HH |
| Cl | DD | H | D | D | H | H | HD |
| Cl | DD | H | D | D | H | H | HH |
| Cl | HD | H | D | D | D | D | DD |
| Cl | HD | H | D | D | D | D | HD |
| Cl | HD | H | D | D | H | D | DD |
| Cl | HD | H | D | D | D | H | DD |
| Cl | HD | H | D | D | D | D | HH |
| Cl | HD | H | D | D | H | D | HD |
| Cl | HD | H | D | D | D | H | HD |
| Cl | HD | H | D | D | H | H | DD |
| Cl | HD | H | D | D | H | D | HH |
| Cl | HD | H | D | D | D | H | HH |
| Cl | HD | H | D | D | H | H | HD |
| Cl | HD | H | D | D | H | H | HH |
| Cl | HH | H | D | D | D | D | DD |
| Cl | HH | H | D | D | D | D | HD |
| Cl | HH | H | D | D | H | D | DD |
| Cl | HH | H | D | D | D | H | DD |
| Cl | HH | H | D | D | D | D | HH |
| Cl | HH | H | D | D | H | D | HD |
| Cl | HH | H | D | D | D | H | HD |
| Cl | HH | H | D | D | H | H | DD |
| Cl | HH | H | D | D | H | D | HH |
| Cl | HH | H | D | D | D | H | HH |
| Cl | HH | H | D | D | H | H | HD |
| Cl | HH | H | D | D | H | H | HH |
| Cl | DD | D | H | D | D | D | DD |
| Cl | DD | D | H | D | D | D | HD |
| Cl | DD | D | H | D | H | D | DD |
| Cl | DD | D | H | D | D | H | DD |
| Cl | DD | D | H | D | D | D | HH |
| Cl | DD | D | H | D | H | D | HD |
| Cl | DD | D | H | D | D | H | HD |
| Cl | DD | D | H | D | H | H | DD |
| Cl | DD | D | H | D | H | D | HH |
| Cl | DD | D | H | D | D | H | HH |
| Cl | DD | D | H | D | H | H | HD |
| Cl | DD | D | H | D | H | H | HH |
| Cl | HD | D | H | D | D | D | DD |
| Cl | HD | D | H | D | D | D | HD |
| Cl | HD | D | H | D | H | D | DD |
| Cl | HD | D | H | D | D | H | DD |
| Cl | HD | D | H | D | D | D | HH |
| Cl | HD | D | H | D | H | D | HD |
| Cl | HD | D | H | D | D | H | HD |
| Cl | HD | D | H | D | H | H | DD |
| Cl | HD | D | H | D | H | D | HH |
| Cl | HD | D | H | D | D | H | HH |
| Cl | HD | D | H | D | H | H | HD |
| Cl | HD | D | H | D | H | H | HH |
| Cl | HH | D | H | D | D | D | DD |
| Cl | HH | D | H | D | D | D | HD |
| Cl | HH | D | H | D | H | D | DD |
| Cl | HH | D | H | D | D | H | DD |
| Cl | HH | D | H | D | D | D | HH |
| Cl | HH | D | H | D | H | D | HD |
| Cl | HH | D | H | D | D | H | HD |
| Cl | HH | D | H | D | H | H | DD |
| Cl | HH | D | H | D | H | D | HH |
| Cl | HH | D | H | D | D | H | HH |
| Cl | HH | D | H | D | H | H | HD |
| Cl | HH | D | H | D | H | H | HH |
| Cl | DD | D | D | H | D | D | DD |
| Cl | DD | D | D | H | D | D | HD |
| Cl | DD | D | D | H | H | D | DD |
| Cl | DD | D | D | H | D | H | DD |
| Cl | DD | D | D | H | D | D | HH |
| Cl | DD | D | D | H | H | D | HD |
| Cl | DD | D | D | H | D | H | HD |
| Cl | DD | D | D | H | H | H | DD |
| Cl | DD | D | D | H | H | D | HH |
| Cl | DD | D | D | H | D | H | HH |
| Cl | DD | D | D | H | H | H | HD |
| Cl | DD | D | D | H | H | H | HH |
| Cl | HD | D | D | H | D | D | DD |
| Cl | HD | D | D | H | D | D | HD |
| Cl | HD | D | D | H | H | D | DD |
| Cl | HD | D | D | H | D | H | DD |
| Cl | HD | D | D | H | D | D | HH |
| Cl | HD | D | D | H | H | D | HD |
| Cl | HD | D | D | H | D | H | HD |
| Cl | HD | D | D | H | H | H | DD |
| Cl | HD | D | D | H | H | D | HH |
| Cl | HD | D | D | H | D | H | HH |
| Cl | HD | D | D | H | H | H | HD |
| Cl | HD | D | D | H | H | H | HH |
| Cl | HH | D | D | H | D | D | DD |
| Cl | HH | D | D | H | D | D | HD |
| Cl | HH | D | D | H | H | D | DD |
| Cl | HH | D | D | H | D | H | DD |
| Cl | HH | D | D | H | D | D | HH |
| Cl | HH | D | D | H | H | D | HD |
| Cl | HH | D | D | H | D | H | HD |
| Cl | HH | D | D | H | H | H | DD |
| Cl | HH | D | D | H | H | D | HH |
| Cl | HH | D | D | H | D | H | HH |
| Cl | HH | D | D | H | H | H | HD |
| Cl | HH | D | D | H | H | H | HH |
| Cl | DD | H | H | D | D | D | DD |
| Cl | DD | H | H | D | D | D | HD |
| Cl | DD | H | H | D | H | D | DD |
| Cl | DD | H | H | D | D | H | DD |
| Cl | DD | H | H | D | D | D | HH |
| Cl | DD | H | H | D | H | D | HD |
| Cl | DD | H | H | D | D | H | HD |
| Cl | DD | H | H | D | H | H | DD |
| Cl | DD | H | H | D | H | D | HH |
| Cl | DD | H | H | D | D | H | HH |
| Cl | DD | H | H | D | H | H | HD |
| Cl | DD | H | H | D | H | H | HH |
| Cl | HD | H | H | D | D | D | DD |
| Cl | HD | H | H | D | D | D | HD |
| Cl | HD | H | H | D | H | D | DD |
| Cl | HD | H | H | D | D | H | DD |
| Cl | HD | H | H | D | D | D | HH |
| Cl | HD | H | H | D | H | D | HD |
| Cl | HD | H | H | D | D | H | HD |
| Cl | HD | H | H | D | H | H | DD |
| Cl | HD | H | H | D | H | D | HH |
| Cl | HD | H | H | D | D | H | HH |
| Cl | HD | H | H | D | H | H | HD |
| Cl | HD | H | H | D | H | H | HH |
| Cl | HH | H | H | D | D | D | DD |
| Cl | HH | H | H | D | D | D | HD |
| Cl | HH | H | H | D | H | D | DD |
| Cl | HH | H | H | D | D | H | DD |
| Cl | HH | H | H | D | D | D | HH |
| Cl | HH | H | H | D | H | D | HD |
| Cl | HH | H | H | D | D | H | HD |
| Cl | HH | H | H | D | H | H | DD |
| Cl | HH | H | H | D | H | D | HH |
| Cl | HH | H | H | D | D | H | HH |
| Cl | HH | H | H | D | H | H | HD |
| Cl | HH | H | H | D | H | H | HH |
| Cl | DD | D | H | H | D | D | DD |
| Cl | DD | D | H | H | D | D | HD |
| Cl | DD | D | H | H | H | D | DD |
| Cl | DD | D | H | H | D | H | DD |
| Cl | DD | D | H | H | D | D | HH |
| Cl | DD | D | H | H | H | D | HD |
| Cl | DD | D | H | H | D | H | HD |
| Cl | DD | D | H | H | H | H | DD |
| Cl | DD | D | H | H | H | D | HH |
| Cl | DD | D | H | H | D | H | HH |
| Cl | DD | D | H | H | H | H | HD |
| Cl | DD | D | H | H | H | H | HH |
| Cl | HD | D | H | H | D | D | DD |
| Cl | HD | D | H | H | D | D | HD |
| Cl | HD | D | H | H | H | D | DD |
| Cl | HD | D | H | H | D | H | DD |
| Cl | HD | D | H | H | D | D | HH |
| Cl | HD | D | H | H | H | D | HD |
| Cl | HD | D | H | H | D | H | HD |
| Cl | HD | D | H | H | H | H | DD |
| Cl | HD | D | H | H | H | D | HH |
| Cl | HD | D | H | H | D | H | HH |
| Cl | HD | D | H | H | H | H | HD |
| Cl | HD | D | H | H | H | H | HH |
| Cl | HH | D | H | H | D | D | DD |
| Cl | HH | D | H | H | D | D | HD |
| Cl | HH | D | H | H | H | D | DD |
| Cl | HH | D | H | H | D | H | DD |
| Cl | HH | D | H | H | D | D | HH |
| Cl | HH | D | H | H | H | D | HD |
| Cl | HH | D | H | H | D | H | HD |
| Cl | HH | D | H | H | H | H | DD |
| Cl | HH | D | H | H | H | D | HH |
| Cl | HH | D | H | H | D | H | HH |
| Cl | HH | D | H | H | H | H | HD |
| Cl | HH | D | H | H | H | H | HH |
| Cl | DD | H | D | H | D | D | DD |
| Cl | DD | H | D | H | D | D | HD |
| Cl | DD | H | D | H | H | D | DD |
| Cl | DD | H | D | H | D | H | DD |
| Cl | DD | H | D | H | D | D | HH |
| Cl | DD | H | D | H | H | D | HD |
| Cl | DD | H | D | H | D | H | HD |
| Cl | DD | H | D | H | H | H | DD |
| Cl | DD | H | D | H | H | D | HH |
| Cl | DD | H | D | H | D | H | HH |
| Cl | DD | H | D | H | H | H | HD |
| Cl | DD | H | D | H | H | H | HH |
| Cl | HD | H | D | H | D | D | DD |
| Cl | HD | H | D | H | D | D | HD |
| Cl | HD | H | D | H | H | D | DD |
| Cl | HD | H | D | H | D | H | DD |
| Cl | HD | H | D | H | D | D | HH |
| Cl | HD | H | D | H | H | D | HD |
| Cl | HD | H | D | H | D | H | HD |
| Cl | HD | H | D | H | H | H | DD |
| Cl | HD | H | D | H | H | D | HH |
| Cl | HD | H | D | H | D | H | HH |
| Cl | HD | H | D | H | H | H | HD |
| Cl | HD | H | D | H | H | H | HH |
| Cl | HH | H | D | H | D | D | DD |
| Cl | HH | H | D | H | D | D | HD |
| Cl | HH | H | D | H | H | D | DD |
| Cl | HH | H | D | H | D | H | DD |
| Cl | HH | H | D | H | D | D | HH |
| Cl | HH | H | D | H | H | D | HD |
| Cl | HH | H | D | H | D | H | HD |
| Cl | HH | H | D | H | H | H | DD |
| Cl | HH | H | D | H | H | D | HH |
| Cl | HH | H | D | H | D | H | HH |
| Cl | HH | H | D | H | H | H | HD |
| Cl | HH | H | D | H | H | H | HH |
| Cl | DD | H | H | H | D | D | DD |
| Cl | DD | H | H | H | D | D | HD |
| Cl | DD | H | H | H | H | D | DD |
| Cl | DD | H | H | H | D | H | DD |
| Cl | DD | H | H | H | D | D | HH |
| Cl | DD | H | H | H | H | D | HD |
| Cl | DD | H | H | H | D | H | HD |
| Cl | DD | H | H | H | H | H | DD |
| Cl | DD | H | H | H | H | D | HH |
| Cl | DD | H | H | H | D | H | HH |
| Cl | DD | H | H | H | H | H | HD |
| Cl | DD | H | H | H | H | H | HH |
| Cl | HD | H | H | H | D | D | DD |
| Cl | HD | H | H | H | D | D | HD |
| Cl | HD | H | H | H | H | D | DD |
| Cl | HD | H | H | H | D | H | DD |
| Cl | HD | H | H | H | D | D | HH |
| Cl | HD | H | H | H | H | D | HD |
| Cl | HD | H | H | H | D | H | HD |
| Cl | HD | H | H | H | H | H | DD |
| Cl | HD | H | H | H | H | D | HH |
| Cl | HD | H | H | H | D | H | HH |
| Cl | HD | H | H | H | H | H | HD |
| Cl | HD | H | H | H | H | H | HH |
| Cl | HH | H | H | H | D | D | DD |
| Cl | HH | H | H | H | D | D | HD |
| Cl | HH | H | H | H | H | D | DD |
| Cl | HH | H | H | H | D | H | DD |
| Cl | HH | H | H | H | D | D | HH |
| Cl | HH | H | H | H | H | D | HD |
| Cl | HH | H | H | H | D | H | HD |
| Cl | HH | H | H | H | H | H | DD |
| Cl | HH | H | H | H | H | D | HH |
| Cl | HH | H | H | H | D | H | HH |
| Cl | HH | H | H | H | H | H | HD |
| Cl | HH | H | H | H | H | H | HH |
| N₃ | DD | D | D | D | D | D | DD |
| N₃ | DD | D | D | D | D | D | HD |
| N₃ | DD | D | D | D | H | D | DD |
| N₃ | DD | D | D | D | D | H | DD |
| N₃ | DD | D | D | D | D | D | HH |
| N₃ | DD | D | D | D | H | D | HD |
| N₃ | DD | D | D | D | D | H | HD |
| N₃ | DD | D | D | D | H | H | DD |
| N₃ | DD | D | D | D | H | D | HH |
| N₃ | DD | D | D | D | D | H | HH |
| N₃ | DD | D | D | D | H | H | HD |
| N₃ | DD | D | D | D | H | H | HH |
| N₃ | HD | D | D | D | D | D | DD |
| N₃ | HD | D | D | D | D | D | HD |
| N₃ | HD | D | D | D | H | D | DD |
| N₃ | HD | D | D | D | D | H | DD |
| N₃ | HD | D | D | D | D | D | HH |
| N₃ | HD | D | D | D | H | D | HD |
| N₃ | HD | D | D | D | D | H | HD |
| N₃ | HD | D | D | D | H | H | DD |
| N₃ | HD | D | D | D | H | D | HH |
| N₃ | HD | D | D | D | D | H | HH |
| N₃ | HD | D | D | D | H | H | HD |
| N₃ | HD | D | D | D | H | H | HH |
| N₃ | HH | D | D | D | D | D | DD |
| N₃ | HH | D | D | D | D | D | HD |
| N₃ | HH | D | D | D | H | D | DD |
| N₃ | HH | D | D | D | D | H | DD |
| N₃ | HH | D | D | D | D | D | HH |
| N₃ | HH | D | D | D | H | D | HD |
| N₃ | HH | D | D | D | D | H | HD |
| N₃ | HH | D | D | D | H | H | DD |
| N₃ | HH | D | D | D | H | D | HH |
| N₃ | HH | D | D | D | D | H | HH |
| N₃ | HH | D | D | D | H | H | HD |
| N₃ | HH | D | D | D | H | H | HH |
| N₃ | DD | H | D | D | D | D | DD |
| N₃ | DD | H | D | D | D | D | HD |
| N₃ | DD | H | D | D | H | D | DD |
| N₃ | DD | H | D | D | D | H | DD |
| N₃ | DD | H | D | D | D | D | HH |
| N₃ | DD | H | D | D | H | D | HD |
| N₃ | DD | H | D | D | D | H | HD |
| N₃ | DD | H | D | D | H | H | DD |
| N₃ | DD | H | D | D | H | D | HH |
| N₃ | DD | H | D | D | D | H | HH |
| N₃ | DD | H | D | D | H | H | HD |
| N₃ | DD | H | D | D | H | H | HH |
| N₃ | HD | H | D | D | D | D | DD |
| N₃ | HD | H | D | D | D | D | HD |
| N₃ | HD | H | D | D | H | D | DD |
| N₃ | HD | H | D | D | D | H | DD |
| N₃ | HD | H | D | D | D | D | HH |
| N₃ | HD | H | D | D | H | D | HD |
| N₃ | HD | H | D | D | D | H | HD |
| N₃ | HD | H | D | D | H | H | DD |
| N₃ | HD | H | D | D | H | D | HH |
| N₃ | HD | H | D | D | D | H | HH |
| N₃ | HD | H | D | D | H | H | HD |
| N₃ | HD | H | D | D | H | H | HH |
| N₃ | HH | H | D | D | D | D | DD |
| N₃ | HH | H | D | D | D | D | HD |
| N₃ | HH | H | D | D | H | D | DD |
| N₃ | HH | H | D | D | D | H | DD |
| N₃ | HH | H | D | D | D | D | HH |
| N₃ | HH | H | D | D | H | D | HD |
| N₃ | HH | H | D | D | D | H | HD |
| N₃ | HH | H | D | D | H | H | DD |
| N₃ | HH | H | D | D | H | D | HH |
| N₃ | HH | H | D | D | D | H | HH |
| N₃ | HH | H | D | D | H | H | HD |
| N₃ | HH | H | D | D | H | H | HH |
| N₃ | DD | D | H | D | D | D | DD |
| N₃ | DD | D | H | D | D | D | HD |
| N₃ | DD | D | H | D | H | D | DD |
| N₃ | DD | D | H | D | D | H | DD |
| N₃ | DD | D | H | D | D | D | HH |
| N₃ | DD | D | H | D | H | D | HD |
| N₃ | DD | D | H | D | D | H | HD |
| N₃ | DD | D | H | D | H | H | DD |
| N₃ | DD | D | H | D | H | D | HH |
| N₃ | DD | D | H | D | D | H | HH |
| N₃ | DD | D | H | D | H | H | HD |
| N₃ | DD | D | H | D | H | H | HH |
| N₃ | HD | D | H | D | D | D | DD |
| N₃ | HD | D | H | D | D | D | HD |
| N₃ | HD | D | H | D | H | D | DD |
| N₃ | HD | D | H | D | D | H | DD |
| N₃ | HD | D | H | D | D | D | HH |
| N₃ | HD | D | H | D | H | D | HD |
| N₃ | HD | D | H | D | D | H | HD |
| N₃ | HD | D | H | D | H | H | DD |
| N₃ | HD | D | H | D | H | D | HH |
| N₃ | HD | D | H | D | D | H | HH |
| N₃ | HD | D | H | D | H | H | HD |
| N₃ | HD | D | H | D | H | H | HH |
| N₃ | HH | D | H | D | D | D | DD |
| N₃ | HH | D | H | D | D | D | HD |
| N₃ | HH | D | H | D | H | D | DD |
| N₃ | HH | D | H | D | D | H | DD |
| N₃ | HH | D | H | D | D | D | HH |
| N₃ | HH | D | H | D | H | D | HD |
| N₃ | HH | D | H | D | D | H | HD |
| N₃ | HH | D | H | D | H | H | DD |
| N₃ | HH | D | H | D | H | D | HH |
| N₃ | HH | D | H | D | D | H | HH |
| N₃ | HH | D | H | D | H | H | HD |
| N₃ | HH | D | H | D | H | H | HH |
| N₃ | DD | D | D | H | D | D | DD |
| N₃ | DD | D | D | H | D | D | HD |
| N₃ | DD | D | D | H | H | D | DD |
| N₃ | DD | D | D | H | D | H | DD |
| N₃ | DD | D | D | H | D | D | HH |
| N₃ | DD | D | D | H | H | D | HD |
| N₃ | DD | D | D | H | D | H | HD |
| N₃ | DD | D | D | H | H | H | DD |
| N₃ | DD | D | D | H | H | D | HH |
| N₃ | DD | D | D | H | D | H | HH |
| N₃ | DD | D | D | H | H | H | HD |
| N₃ | DD | D | D | H | H | H | HH |
| N₃ | HD | D | D | H | D | D | DD |
| N₃ | HD | D | D | H | D | D | HD |
| N₃ | HD | D | D | H | H | D | DD |
| N₃ | HD | D | D | H | D | H | DD |
| N₃ | HD | D | D | H | D | D | HH |
| N₃ | HD | D | D | H | H | D | HD |
| N₃ | HD | D | D | H | D | H | HD |
| N₃ | HD | D | D | H | H | H | DD |
| N₃ | HD | D | D | H | H | D | HH |
| N₃ | HD | D | D | H | D | H | HH |
| N₃ | HD | D | D | H | H | H | HD |
| N₃ | HD | D | D | H | H | H | HH |
| N₃ | HH | D | D | H | D | D | DD |
| N₃ | HH | D | D | H | D | D | HD |
| N₃ | HH | D | D | H | H | D | DD |
| N₃ | HH | D | D | H | D | H | DD |
| N₃ | HH | D | D | H | D | D | HH |
| N₃ | HH | D | D | H | H | D | HD |
| N₃ | HH | D | D | H | D | H | HD |
| N₃ | HH | D | D | H | H | H | DD |
| N₃ | HH | D | D | H | H | D | HH |
| N₃ | HH | D | D | H | D | H | HH |
| N₃ | HH | D | D | H | H | H | HD |
| N₃ | HH | D | D | H | H | H | HH |
| N₃ | DD | H | H | D | D | D | DD |
| N₃ | DD | H | H | D | D | D | HD |
| N₃ | DD | H | H | D | H | D | DD |
| N₃ | DD | H | H | D | D | H | DD |
| N₃ | DD | H | H | D | D | D | HH |
| N₃ | DD | H | H | D | H | D | HD |
| N₃ | DD | H | H | D | D | H | HD |
| N₃ | DD | H | H | D | H | H | DD |
| N₃ | DD | H | H | D | H | D | HH |
| N₃ | DD | H | H | D | D | H | HH |
| N₃ | DD | H | H | D | H | H | HD |
| N₃ | DD | H | H | D | H | H | HH |
| N₃ | HD | H | H | D | D | D | DD |
| N₃ | HD | H | H | D | D | D | HD |
| N₃ | HD | H | H | D | H | D | DD |
| N₃ | HD | H | H | D | D | H | DD |
| N₃ | HD | H | H | D | D | D | HH |
| N₃ | HD | H | H | D | H | D | HD |
| N₃ | HD | H | H | D | D | H | HD |
| N₃ | HD | H | H | D | H | H | DD |
| N₃ | HD | H | H | D | H | D | HH |
| N₃ | HD | H | H | D | D | H | HH |
| N₃ | HD | H | H | D | H | H | HD |
| N₃ | HD | H | H | D | H | H | HH |
| N₃ | HH | H | H | D | D | D | DD |
| N₃ | HH | H | H | D | D | D | HD |
| N₃ | HH | H | H | D | H | D | DD |
| N₃ | HH | H | H | D | D | H | DD |
| N₃ | HH | H | H | D | D | D | HH |
| N₃ | HH | H | H | D | H | D | HD |
| N₃ | HH | H | H | D | D | H | HD |
| N₃ | HH | H | H | D | H | H | DD |
| N₃ | HH | H | H | D | H | D | HH |
| N₃ | HH | H | H | D | D | H | HH |
| N₃ | HH | H | H | D | H | H | HD |
| N₃ | HH | H | H | D | H | H | HH |
| N₃ | DD | D | H | H | D | D | DD |
| N₃ | DD | D | H | H | D | D | HD |
| N₃ | DD | D | H | H | H | D | DD |
| N₃ | DD | D | H | H | D | H | DD |
| N₃ | DD | D | H | H | D | D | HH |
| N₃ | DD | D | H | H | H | D | HD |
| N₃ | DD | D | H | H | D | H | HD |
| N₃ | DD | D | H | H | H | H | DD |
| N₃ | DD | D | H | H | H | D | HH |
| N₃ | DD | D | H | H | D | H | HH |
| N₃ | DD | D | H | H | H | H | HD |
| N₃ | DD | D | H | H | H | H | HH |
| N₃ | HD | D | H | H | D | D | DD |
| N₃ | HD | D | H | H | D | D | HD |
| N₃ | HD | D | H | H | H | D | DD |
| N₃ | HD | D | H | H | D | H | DD |
| N₃ | HD | D | H | H | D | D | HH |
| N₃ | HD | D | H | H | H | D | HD |
| N₃ | HD | D | H | H | D | H | HD |
| N₃ | HD | D | H | H | H | H | DD |
| N₃ | HD | D | H | H | H | D | HH |
| N₃ | HD | D | H | H | D | H | HH |
| N₃ | HD | D | H | H | H | H | HD |
| N₃ | HD | D | H | H | H | H | HH |
| N₃ | HH | D | H | H | D | D | DD |
| N₃ | HH | D | H | H | D | D | HD |
| N₃ | HH | D | H | H | H | D | DD |
| N₃ | HH | D | H | H | D | H | DD |
| N₃ | HH | D | H | H | D | D | HH |
| N₃ | HH | D | H | H | H | D | HD |
| N₃ | HH | D | H | H | D | H | HD |
| N₃ | HH | D | H | H | H | H | DD |
| N₃ | HH | D | H | H | H | D | HH |
| N₃ | HH | D | H | H | D | H | HH |
| N₃ | HH | D | H | H | H | H | HD |
| N₃ | HH | D | H | H | H | H | HH |
| N₃ | DD | H | D | H | D | D | DD |
| N₃ | DD | H | D | H | D | D | HD |
| N₃ | DD | H | D | H | H | D | DD |
| N₃ | DD | H | D | H | D | H | DD |
| N₃ | DD | H | D | H | D | D | HH |
| N₃ | DD | H | D | H | H | D | HD |
| N₃ | DD | H | D | H | D | H | HD |
| N₃ | DD | H | D | H | H | H | DD |
| N₃ | DD | H | D | H | H | D | HH |
| N₃ | DD | H | D | H | D | H | HH |
| N₃ | DD | H | D | H | H | H | HD |
| N₃ | DD | H | D | H | H | H | HH |
| N₃ | HD | H | D | H | D | D | DD |
| N₃ | HD | H | D | H | D | D | HD |
| N₃ | HD | H | D | H | H | D | DD |
| N₃ | HD | H | D | H | D | H | DD |
| N₃ | HD | H | D | H | D | D | HH |
| N₃ | HD | H | D | H | H | D | HD |
| N₃ | HD | H | D | H | D | H | HD |
| N₃ | HD | H | D | H | H | H | DD |
| N₃ | HD | H | D | H | H | D | HH |
| N₃ | HD | H | D | H | D | H | HH |
| N₃ | HD | H | D | H | H | H | HD |
| N₃ | HD | H | D | H | H | H | HH |
| N₃ | HH | H | D | H | D | D | DD |
| N₃ | HH | H | D | H | D | D | HD |
| N₃ | HH | H | D | H | H | D | DD |
| N₃ | HH | H | D | H | D | H | DD |
| N₃ | HH | H | D | H | D | D | HH |
| N₃ | HH | H | D | H | H | D | HD |
| N₃ | HH | H | D | H | D | H | HD |
| N₃ | HH | H | D | H | H | H | DD |
| N₃ | HH | H | D | H | H | D | HH |
| N₃ | HH | H | D | H | D | H | HH |
| N₃ | HH | H | D | H | H | H | HD |
| N₃ | HH | H | D | H | H | H | HH |
| N₃ | DD | H | H | H | D | D | DD |
| N₃ | DD | H | H | H | D | D | HD |
| N₃ | DD | H | H | H | H | D | DD |
| N₃ | DD | H | H | H | D | H | DD |
| N₃ | DD | H | H | H | D | D | HH |
| N₃ | DD | H | H | H | H | D | HD |
| N₃ | DD | H | H | H | D | H | HD |
| N₃ | DD | H | H | H | H | H | DD |
| N₃ | DD | H | H | H | H | D | HH |
| N₃ | DD | H | H | H | D | H | HH |
| N₃ | DD | H | H | H | H | H | HD |
| N₃ | DD | H | H | H | H | H | HH |
| N₃ | HD | H | H | H | D | D | DD |
| N₃ | HD | H | H | H | D | D | HD |
| N₃ | HD | H | H | H | H | D | DD |
| N₃ | HD | H | H | H | D | H | DD |
| N₃ | HD | H | H | H | D | D | HH |
| N₃ | HD | H | H | H | H | D | HD |
| N₃ | HD | H | H | H | D | H | HD |
| N₃ | HD | H | H | H | H | H | DD |
| N₃ | HD | H | H | H | H | D | HH |
| N₃ | HD | H | H | H | D | H | HH |
| N₃ | HD | H | H | H | H | H | HD |
| N₃ | HD | H | H | H | H | H | HH |
| N₃ | HH | H | H | H | D | D | DD |
| N₃ | HH | H | H | H | D | D | HD |
| N₃ | HH | H | H | H | H | D | DD |
| N₃ | HH | H | H | H | D | H | DD |
| N₃ | HH | H | H | H | D | D | HH |
| N₃ | HH | H | H | H | H | D | HD |
| N₃ | HH | H | H | H | D | H | HD |
| N₃ | HH | H | H | H | H | H | DD |
| N₃ | HH | H | H | H | H | D | HH |
| N₃ | HH | H | H | H | D | H | HH |
| N₃ | HH | H | H | H | H | H | HD |
| N₃ | HH | H | H | H | H | H | HH |

In some embodiments of Table A, R¹ can be hydrogen. In some embodiments of Table A, R¹ can be deuterium. In still other embodiments of Table A, R¹ can be an optionally substituted acyl, for example, R¹ can be —C(═O)C₁₋₆ alkyl. In some embodiments of Table A, R³ can be OH. In other embodiments of Table A, R³ can be —OC(═O)R^{A1}. In some embodiments of Table A, R¹ can be hydrogen and R³ can be OH. In other embodiments of Table A, R¹ can be an optionally substituted acyl and R³ can be —OC(═O)R^{A1}. In some embodiments of Table A, R¹ can be —C(═O)C₁₋₆ alkyl and R³ can be —OC(═O)C₁₋₆ alkyl. In some embodiments of Table A, R¹ can be

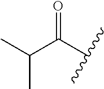 [SMILES: *C(=O)C(C)C]

### and R³ can be

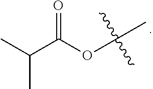 [SMILES: *OC(=O)C(C)C]

In some embodiments, R¹ and/or R³ can include one or more deuterium atoms. For example, R¹ can be deuterium or R¹ can be

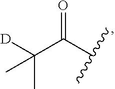 [SMILES: *C(=O)C([2H])(C)C]

and/or R³ can be

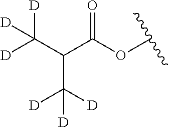 [SMILES: *OC(=O)C(C([2H])([2H])[2H])C([2H])([2H])[2H]]

### or R³ can be OD.

Compound (A), or a pharmaceutically acceptable salt thereof, can act as a chain-terminator and inhibit replication of a virus, such as a paramyxovirus.

Examples of Compound (A), or a pharmaceutically acceptable salt thereof, include the following:

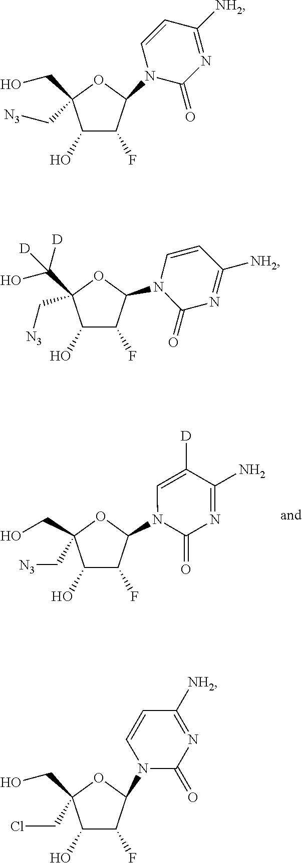 [SMILES: [N-]=[N+]=NC[C@]1(CO)O[C@@H](n2ccc(N)nc2=O)[C@H](F)[C@@H]1O ; [2H]C([2H])(O)[C@@]1(CN=[N+]=[N-])O[C@@H](n2ccc(N)nc2=O)[C@H](F)[C@@H]1O ; [2H]c1cn([C@@H]2O[C@@](CO)(CN=[N+]=[N-])[C@@H](O)[C@H]2F)c(=O)nc1N ; Nc1ccn([C@@H]2O[C@@](CO)(CCl)[C@@H](O)[C@H]2F)c(=O)n1]

or a pharmaceutically acceptable salt of any of the foregoing.

Further examples of Compound (A), or a pharmaceutically acceptable salt thereof, include:

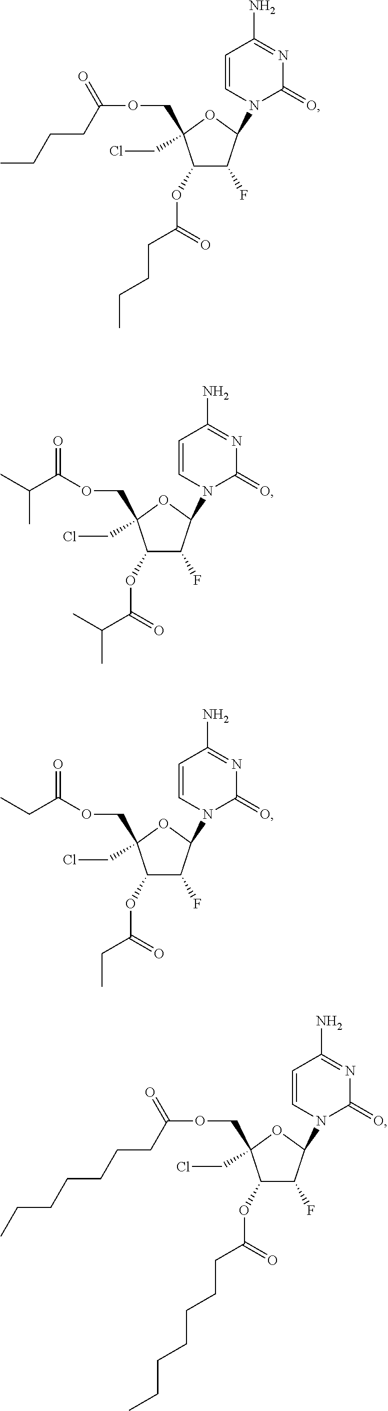 [SMILES: CCCCC(=O)OC[C@@]1(CCl)O[C@@H](n2ccc(N)nc2=O)[C@H](F)[C@@H]1OC(=O)CCCC ; CC(C)C(=O)OC[C@@]1(CCl)O[C@@H](n2ccc(N)nc2=O)[C@H](F)[C@@H]1OC(=O)C(C)C ; CCC(=O)OC[C@@]1(CCl)O[C@@H](n2ccc(N)nc2=O)[C@H](F)[C@@H]1OC(=O)CC ; CCCCCCCC(=O)OC[C@@]1(CCl)O[C@@H](n2ccc(N)nc2=O)[C@H](F)[C@@H]1OC(=O)CCCCCCC]

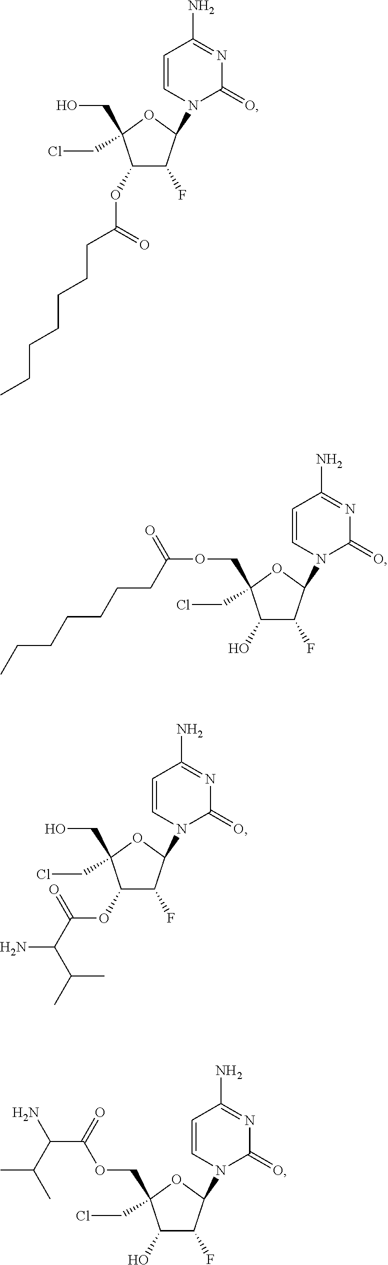 [SMILES: CCCCCCCC(=O)O[C@H]1[C@@H](F)[C@H](n2ccc(N)nc2=O)O[C@@]1(CO)CCl ; CCCCCCCC(=O)OC[C@@]1(CCl)O[C@@H](n2ccc(N)nc2=O)[C@H](F)[C@@H]1O ; CC(C)C(N)C(=O)O[C@H]1[C@@H](F)[C@H](n2ccc(N)nc2=O)O[C@@]1(CO)CCl ; CC(C)C(N)C(=O)OC[C@@]1(CCl)O[C@@H](n2ccc(N)nc2=O)[C@H](F)[C@@H]1O]

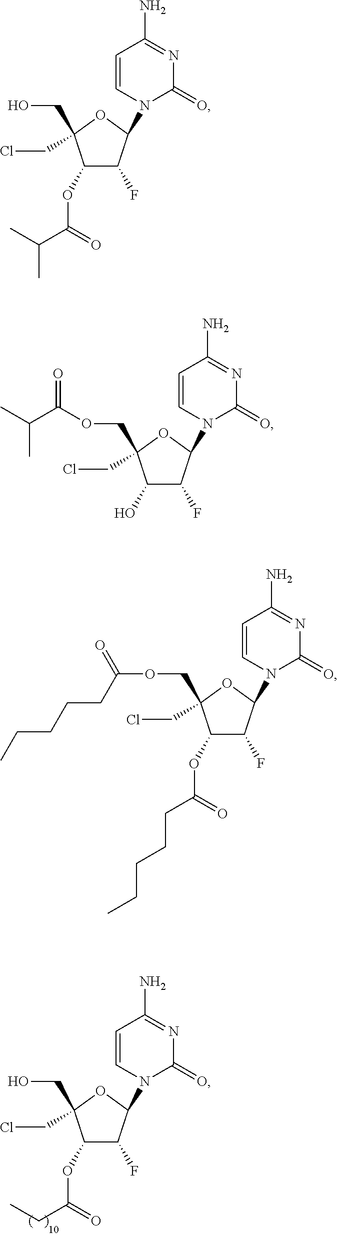

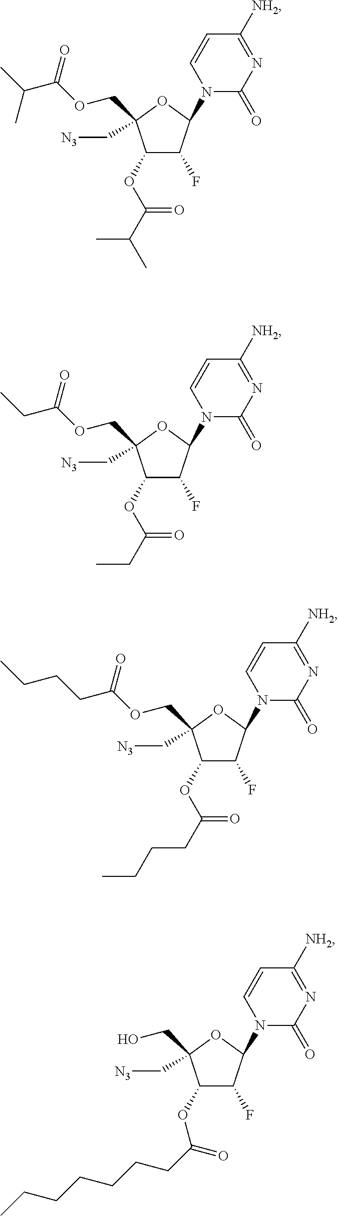 [SMILES: CC(C)C(=O)OC[C@@]1(CN=[N+]=[N-])O[C@@H](n2ccc(N)nc2=O)[C@H](F)[C@@H]1OC(=O)C(C)C ; CCC(=O)OC[C@@]1(CN=[N+]=[N-])O[C@@H](n2ccc(N)nc2=O)[C@H](F)[C@@H]1OC(=O)CC ; CCCCC(=O)OC[C@@]1(CN=[N+]=[N-])O[C@@H](n2ccc(N)nc2=O)[C@H](F)[C@@H]1OC(=O)CCCC ; CCCCCCCC(=O)O[C@H]1[C@@H](F)[C@H](n2ccc(N)nc2=O)O[C@@]1(CO)CN=[N+]=[N-]]

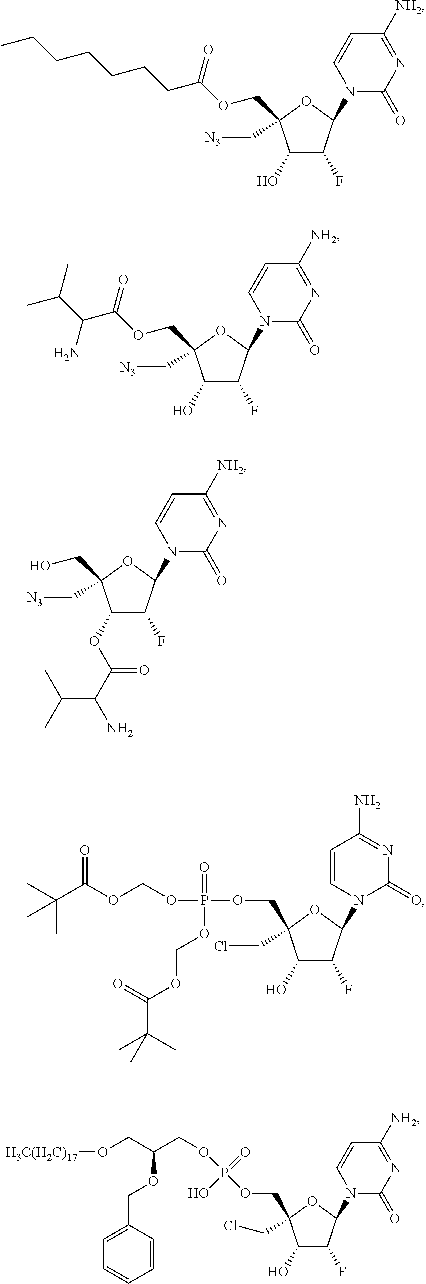

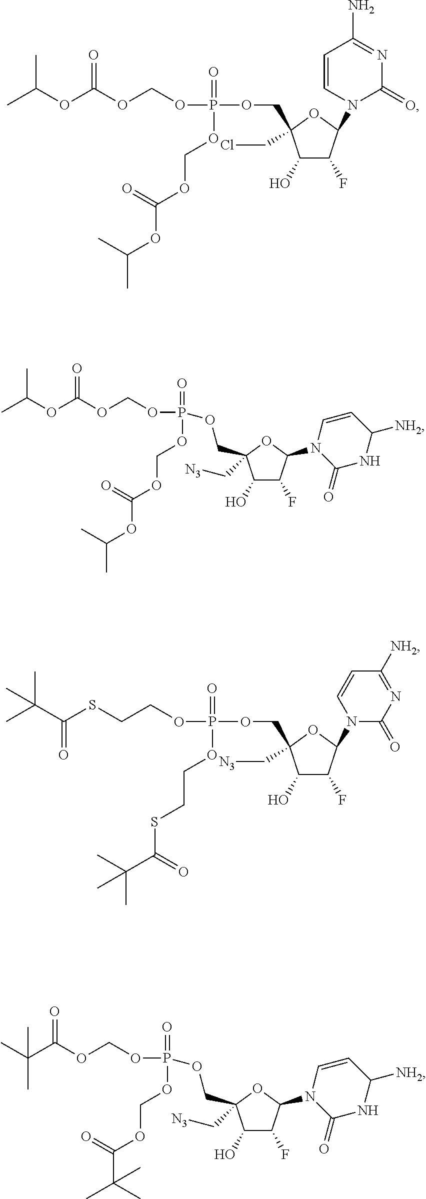 [SMILES: CC(C)OC(=O)OCOP(=O)(OCOC(=O)OC(C)C)OC[C@@]1(CCl)O[C@@H](n2ccc(N)nc2=O)[C@H](F)[C@@H]1O ; CC(C)OC(=O)OCOP(=O)(OCOC(=O)OC(C)C)OC[C@@]1(CN=[N+]=[N-])O[C@@H](N2C=CC(N)NC2=O)[C@H](F)[C@@H]1O ; CC(C)(C)C(=O)SCCOP(=O)(OCCSC(=O)C(C)(C)C)OC[C@@]1(CN=[N+]=[N-])O[C@@H](n2ccc(N)nc2=O)[C@H](F)[C@@H]1O ; CC(C)(C)C(=O)OCOP(=O)(OCOC(=O)C(C)(C)C)OC[C@@]1(CN=[N+]=[N-])O[C@@H](N2C=CC(N)NC2=O)[C@H](F)[C@@H]1O]

or a pharmaceutically acceptable salt of any of the foregoing.

Additional examples of Compound (A), or a pharmaceutically acceptable salt thereof, include the following:

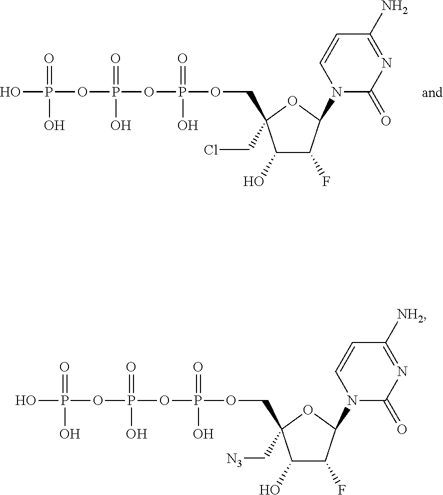 [SMILES: Nc1ccn([C@@H]2O[C@](CCl)(COP(=O)(O)OP(=O)(O)OP(=O)(O)O)[C@@H](O)[C@H]2F)c(=O)n1 ; [N-]=[N+]=NC[C@]1(COP(=O)(O)OP(=O)(O)OP(=O)(O)O)O[C@@H](n2ccc(N)nc2=O)[C@H](F)[C@@H]1O]

or a pharmaceutically acceptable salt of any of the foregoing.

### Compound (B)

A variety of compounds can be compound (B), or a pharmaceutically acceptable salt thereof. In some embodiments, compound (B), or a pharmaceutically acceptable salt thereof, can be selected from an anti-RSV antibody, a fusion protein inhibitor, an N-protein inhibitor, a RSV polymerase inhibitor, an IMPDH inhibitor, an interferon and an other compound that inhibits the RSV virus, or a pharmaceutically acceptable salt of any of the foregoing.

In some embodiments, compound (B), or a pharmaceutically acceptable salt thereof, can be an anti-RSV agent. In some embodiments, compound (B) can be an anti-RSV antibody, or a pharmaceutically acceptable salt thereof. Examples of anti-RSV antibodies include, but are not limited to, RSV-IGIV (RespiGam®), palivizumab (Synagis®, a chimeric humanized IgG monoclonal antibody) and motavizumab (MEDI-524, humanized monoclonal antibody), and pharmaceutically acceptable salts of the foregoing.

In some embodiments, compound (B) can be a fusion protein inhibitor, or a pharmaceutically acceptable salt thereof. A non-limiting list of fusion protein inhibitors include the following: 1-cyclopropyl-3-[[1-(4-hydroxybutyl)benzimidazol-2-yl]methyl]imidazo[4,5-c]pyridin-2-one (BMS-433771), 4,4″-bis-{4,6-bis-[3-(bis-carbamoylmethyl-sulfamoyl)-phenylamino]-(1,3,5)triazin-2-ylamino}-biphenyl-2,2″-disulfonic-acid (RFI-641), 4,4′-Bis[4,6-di[3-aminophenyl-N,N-bis(2-carbamoylethyl)-sulfonilimino]-1,3,5-triazine-2-ylamino]-biphenyl-2,2′-disulfonic acid, disodium salt (CL387626), 2-[[2-[[1-(2-aminoethyl)-4-piperidinyl]amino]-4-methyl-1H-benzimidazol-1-yl]-6-methyl-3-pyridinol (JNJ-2408068), 2-[[6-[[[2-(3-Hydroxypropyl)-5-methylphenyl]amino]methyl]-2-[[3-(morpholin-4-yl)propyl]amino]benzimidazol-1-yl]methyl]-6-methylpyridin-3-ol (TMC-353121), 5,5′-bis[1-(((5-amino-1H-tetrazolyl)imino)methyl)]2,2′,4″-methylidynetrisphenol (VP-14637, MDT-637), N-(2-hydroxyethyl)-4-methoxy-N-methyl-3-(6-methyl-[1,2,4]triazolo[3,4-a]phthalazin-3-yl)benzenesulfonamide (P13), 2-((2-((1-(2-aminoethyl)piperidin-4-yl)amino)-4-methyl-1H-benzo[d]imidazol-1-yl)methyl)-6-methylpyridin-3-ol (R170591), 1,4-bis(3-methylpyridin-4-yl)-1,4-diazepane (C15), (R)-9b-(4-chlorophenyl)-1-(4-fluorobenzoyl)-2,3-dihydro-1H-imidazo[1′,2′:1,2]pyrrolo[3,4-c]pyridin-5 (9bH)-one (BTA9981), [2,2-bis(docosyloxy-oxymethyl)propyl-5-acetaoamido-3,5-dideoxy-4,7,8,9-tetra-O-(sodium-oxysulfonyl)-D-glycero-D-galacto-2-nonulopyranosid]onate (MBX-300), BTA-C286, N-(2-((S)-2-(5-((S)-3-aminopyrrolidin-1-yl)-6-methylpyrazolo[1,5-a]pyrimidin-2-yl)piperidine-1-carbonyl)-4-chlorophenyl)methanesulfonamide (GS-5806), an anti-RSV nanobody (e.g., ALX-0171 (a trivalent nanobody, for example, those described in U.S. Publication No. 2012/0128669, filed Jun. 7, 2010, which is hereby incorporated by reference for the limited purpose of its description of nanobodies), Ablynx) and a peptide fusion inhibitor (such as a peptide having the sequence DEFDASISQVNEKINQSLAFIRKSDELL (T-67, SEQ ID NO: 1, U.S. Pat. No. 6,623,741, filed Feb. 29, 2000), and a peptide having the sequence FDASISQVNEKINQSLAFIRKSDELLHNVNAGKST (T-118, SEQ ID NO: 2, U.S. Pat. No. 6,623,741, filed Feb. 29, 2000), and pharmaceutically acceptable salts of the foregoing. U.S. Pat. No. 6,623,741 is hereby incorporated by reference for the limited purpose of its description of peptide fusion inhibitors.

In some embodiments, compound (B) can be an N-protein inhibitor, or a pharmaceutically acceptable salt thereof. An exemplary N-protein inhibitor is (S)-1-(2-fluorophenyl)-3-(2-oxo-5-phenyl-2,3-dihydro-1H-benzo[e][1,4]diazepin-3-yl)urea (RSV-604), STP-92 (siRNA delivered through nanoparticle based delivery systems, Sirnaomics) and iKT-041 (Inhibikase), and a pharmaceutically acceptable salt thereof

In some embodiments, compound (B) can be a RSV polymerase inhibitor, or a pharmaceutically acceptable salt thereof. Examples of RSV polymerase inhibitors include, but are not limited to, 6-{4-[(biphenyl-2-ylcarbonyl)amino]benzoyl}-N-cyclopropyl-5,6-dihydro-4H-thieno[3,2-d][1]benzazepine-2-carboxamide (YM-53403), N-cyclopropyl-5-(4-(2-(pyrrolidin-1-yl)benzamido)benzoyl)-5,6,7,10-tetrahydrobenzo[b]cyclopenta[d]azepine-9-carboxamide, 6-(4-(2-(2-oxa-7-azaspiro[3.5]nonan-7-yl)nicotinamido)benzoyl)-N-cyclopropyl-5,6-dihydro-4H-benzo[b]thieno[2,3-d]azepine-2-carboxamide, 4-amino-8-(3-{[2-(3,4-dimethoxyphenyl)ethyl]amino}propyl)-6,6-dimethyl-2-(4-methyl-3-nitrophenyl)-1H-imidazo[4,5-h]-isoquinoline-7,9(6H,8H)-dione (CAS Reg. No. 851658-10-1) and 6-(4-(2-(2-oxa-7-azaspiro[3.5]nonan-7-yl)nicotinamido)benzoyl)-N-cyclopropyl-5,6-dihydro-4H-benzo[b]thieno[2,3-d]azepine-2-carboxamide (AZ27), and pharmaceutically acceptable salts of the foregoing.

In some embodiments, compound (B) can be an IMPDH inhibitor, or a pharmaceutically acceptable salt thereof. A non-limiting list of IMPDH inhibitors include: ribavirin, 5-ethynyl-1-beta-D-ribofuranosylimidazole-4-carboxamide (EICAR), 4-hydroxy-3-beta-D-ribofuranosylpyrazole-5-carboxamide (pyrazofurin), 1-((2R,3R,4S,5R)-3,4-dihydroxy-5-(hydroxymethyl)tetrahydrofuran-2-yl)-1H-1,2,4-triazole-3-carboximidamide (Taribavirin, viramidine), 1,3,4-thiadiazol-2-ylcyanamide (LY253963), tetrahydrofuran-3-yl-3-(3-(3-methoxy-4-(oxazol-5-yl)phenyl)ureido)benzylcarbamate (VX-497), (4E)-6-(4-Hydroxy-6-methoxy-7-methyl-3-oxo-1,3-dihydro-2-benzofuran-5-yl)-4-methylhex-4-enoic acid (Mycophenolic acid) and 2-morpholin-4-ylethyl-(E)-6-(4-hydroxy-6-methoxy-7-methyl-3-oxo-1H-2-benzofuran-5-yl)-4-methylhex-4-enoate (Mycophenolate Mofetil), or a pharmaceutically acceptable salt of any of the foregoing.

In some embodiments, compound (B) can be an interferon, or a pharmaceutically acceptable salt thereof. Examples of interferons are described herein. In some embodiments, the interferon can be a pegylated interferon. In some embodiments, the interferon can be a Type 1 interferon, for example, an alpha-interferon (IFN-α). Exemplary alpha-interferons include Pegylated interferon-alpha-2a (PEGASYS®), Pegylated interferon-alpha-2b (PEG-INTRON®) and interferon alfacon-1 (INFERGEN®). In other embodiments, the Type 1 interferon can be a beta-interferon (IFN-β). In some embodiments, the interferon can be a Type 2 interferon. In other embodiments, the interferon can be Type 3 interferon, such as a lambda-interferon (IFN-λ) and pegylated interferon lambda.

In some embodiments, compound (B) can be an other compound that inhibits the RSV virus, or a pharmaceutically acceptable salt thereof. Examples of other compounds that inhibits the RSV virus include, but are not limited to, a double stranded RNA oligonucleotide, 5-methyl-N-[4-(trifluoromethyl)phenyl]-isoxazole-4-carboxamide (leflumomide), N-(2-chloro-4-methylphenyl)-2-((1-(4-methoxyphenyl)-1H-benzo[d]imidazol-2-yl)thio)propanamide (JMN3-003), Medi-559, Medi-534, Medi-557, an intratracheal formulation of recombinant human CC10 (CG-100), high titer, human immunoglobulin (RI-001, ADMA Biologics Inc.) and a non-neutralizing mAb against the G protein (mAb 131-2G), or a pharmaceutically acceptable salt of any of the foregoing. A non-limiting list of double stranded RNA oligonucleotides are ALN-RSV01 (an siRNA agent with the sense strand sequence (5′ to 3′) GGCUCUUAGCAAAGUCAAGdTdT (SEQ ID NO. 3) and the antisense strand sequence (5′ to 3′) CUUGACUUUGCUAAGAGCCdTdT (SEQ ID NO. 4) and ALN-RSV02. Additional information regarding ALN-RSV01 and/or ALN-RSVO2 can be found in U.S. Publication No. 2009/0238772, filed Dec. 15, 2008 (Alnylam Pharmaceuticals).

Additional compounds for Compound (B) include compounds that can be encompassed by the following formulae/compounds. For each of the following formulae/compounds, each variable pertains only to each individual section. For example for Compounds of Formula (B1), the variables listed under Compounds of Formula (B1) refer only to Compounds of Formula (B1) and not Compounds of Formula (B2) or any of the other formulae/compounds provided in this section, unless stated otherwise.

### Compounds of Formula (B1)

Compounds of the general Formula (B1) are described in PCT Publication No. WO 2013/186333, published Dec. 19, 2013, which is hereby incorporated by reference in its entirety. Formula (B1) has the structure:

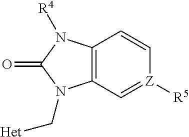

or a stereoisomeric form thereof, wherein: Het can be a heterocycle having formula (b), (c), (d) or (e):

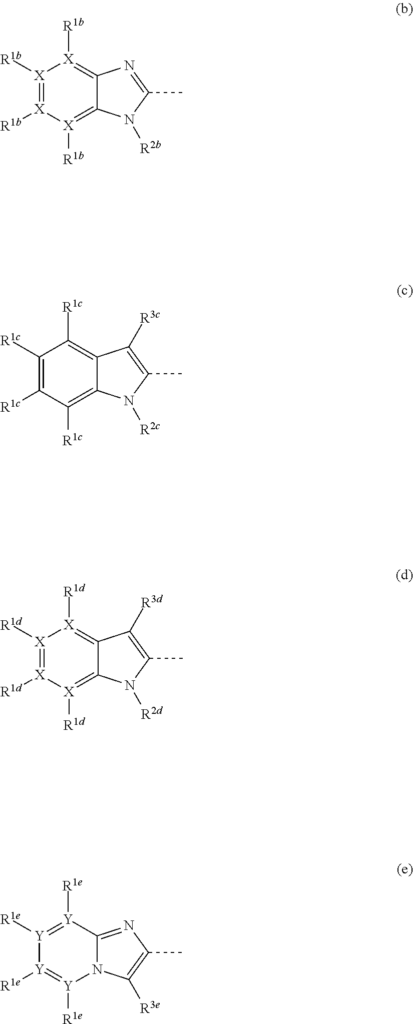

each X independently can be C or N; provided that at least one X is N; R^{1b} can be present when Het has formula (b) and X is C; each R^{1b} can be selected independently from H, halogen, C₁-C₆ alkyl, C₃-C₇ cycloalkyl, C₁-C₆ alkyloxy, N(R⁶)₂, CO(R⁷), CH₂NH₂, CH₂OH, CN, C(═NOH)NH₂, C(═NOCH₃)NH₂, C(═NH)NH₂, CF₃, CF₃, B(OH)₂ and B(O—C₁-C₆ alkyl)₂; R^{1b} is absent when the X to which it is bound is N; R^{2b} can be —(CR⁸R⁹)ₘ—R^{10b}; each R⁶ can be independently selected from H, C₁-C₆ alkyl, COOCH₃ and CONHSO₂CH₃; each R⁷ can be independently selected from OH, C₁-C₆ alkyloxy, NH₂, NHSO₂N(C₁-C₆ alkyl)₂, NHSO₂NHCH₃, NHSO₂(C₁-C₆ alkyl), NHSO₂(C₃-C₇ cycloalkyl) and N(C₁-C₆ alkyl)₂; each R⁸ and R⁹ can be independently chosen from H, C₁-C₁₀ alkyl and C₃-C₇ cycloalkyl; or R⁸ and R⁹ can be taken together form a 4 to 6 membered aliphatic ring that optionally contains one or more heteroatoms selected from N, S and O; R^{10b} can be selected from H, R¹¹, OH, CN, F, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, OCOR⁸, O-Benzyl, NR⁸SO₂R⁹, SO₂NR⁸R⁹, SO₂R⁸, OCONR⁸R⁹, OCONR⁸R¹², N(R⁸)CON(R⁸R⁹), N(R⁸)COOR¹², and a 4 to 6 membered saturated ring containing one oxygen atom; m can be an integer from 2 to 6; R¹¹ can be selected from C₁-C₆ alkyl, C₃-C₇ cycloalkyl, phenyl, pyridinyl and pyrazolyl; each optionally substituted with one or more substituents each independently selected from CF₃, CH₃, OCH₃, OCF₃ and halogen; R¹² can be selected from phenyl, pyridinyl and pyrazolyl; each optionally substituted with one or more substituents each independently selected from CF₃, CH₃, OCH₃, OCF₃ and halogen; or R¹² can be C₁-C₆ alkyl or C₃-C₇ cycloalkyl; each substituted with one or more substituents each independently selected from CF₃, CH₃, OCH₃, OCF₃ and halogen; R^{1c} can be present when Het has formula (c); each R^{1c} can be selected independently from H, halogen, C₁-C₆ alkyl, C₃-C₇ cycloalkyl, C₁-C₆ alkyloxy, N(R⁶)₂, CO(R^{7c}), CH₂NH₂, CH₂OH, CN, C(═NOH)NH₂, C(═NOCH₃)NH₂, C(═NH)NH₂, CF₃, OCF₃, B(OH)₂ and B(O—C₁-C₆ alkyl)₂; R^{1c} can be selected from H, halogen, C₁-C₆ alkyl, C₃-C₇ cycloalkyl, C₁-C₆ alkyloxy and CO(R^{7c}); R^{2c} can be —(CR⁸R⁹)ₘ—R^{10c}; R^{7c} can be selected from OH, O(C₁-C₆ alkyl), NH₂, NHSO₂N(C₁-C₆ alkyl)₂, NHSO₂NHCH₃, NHSO₂(C₁-C₆ alkyl), NHSO₂(C₃-C₇ cycloalkyl), N(C₁-C₆ alkyl)₂, NR⁸R⁹ and NR⁹R^{10c}; R^{10c} can be selected from H, R¹¹, OH, CN, F, CF₂H, CF₃, C(═NOH)NH₂, CONR⁸R⁹, COOR⁸, CONR⁸SO₂R⁹, CON(R⁸)SO₂N(R⁸R⁹), NR⁸R⁹, NR⁸COOR⁹, OCOR⁸, NR⁸SO₂R⁹, SO₂NR⁸R⁹, SO₂R⁸ and a 4 to 6 membered saturated ring containing one oxygen atom; R^{1d} can be present when Het has formula (d) and X is C; each R^{1d} is selected independently from H, OH, halogen, C₁-C₆ alkyl, C₃-C₇ cycloalkyl, C₁-C₆ alkyloxy, N(R⁶)₂, CO(R⁷), CH₂NH₂, CH₂OH, C(═NOH)NH₂, C(═NOCH₃)NH₂, C(═NH)NH₂, CF₃, OCF₃, B(OH)₂ and B(O—C₁-C₆ alkyl)₂; R^{1d} is absent when the X to which it is bound is N; R^{ad} can be selected from H, halogen, C₁-C₆ alkyl, C₃-C₇ cycloalkyl, C₁-C₆ alkyloxy, and CO(R⁷); R^{3d} can be —(CR⁸R⁹)ₘ^{10d}; R^{10d} can be selected from H, R¹¹, OH, CN, F, CF₂H, CF₃, CONR⁸R⁹, COOR⁸, CONR⁸SO₂R⁹, CON(R⁸)SO₂N(R⁸R⁹), NR⁸R⁹, NR⁸COOR₉, OCOR⁸, NR⁸SO₂R⁹, SO₂NR⁸R⁹, SO₂R⁸ and a 4 to 6 membered saturated ring containing one oxygen atom; each Y independently can be C or N; R^{1e} can be present when Het has formula (e) and Y is C; each R^{1e} can be selected independently from H, halogen, C₁-C₆ alkyl, C₃-C₇ cycloalkyl, C₁-C₆ alkyloxy, N(R⁶)₂, CO(R⁷), CH₂NH₂, CH₂OH, CN, C(═NOH)NH₂, C(═NOCH₃)NH₂, C(═NH)NH₂, CF₃, OCF₃, B(OH)₂ and B(O—C₁-C₆ alkyl)₂; R^{1e} is absent when the Y to which it is bound is N; R^{3e} can be selected from H, halogen, —(CR⁸R⁹)ₘ—C≡C—CH₂, can be selected from H, R¹¹, C₁-C₆ alkyloxy, OH, CN, F, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, OCOR⁸, NR⁸SO₂R⁹, SO₂NR⁸R⁹, SO₂R⁸ and a 4 to 6 membered saturated ring containing one oxygen atom; R⁴ can be selected from tert-butyl, Het¹, aryl, Het², CH(CH₃)(CF₃), and C₃-C₇ cycloalkyl substituted with one or more substituents selected from halo and C₁-C₄ alkyl; aryl can represents phenyl or naphthalenyl; said aryl optionally being substituted with one or more substituents each independently selected from halo C₁-C₄ alkyloxy, C₁-C₄ alkyl, OH, CN, CF₂H, CF₃, CF₃O, CONR⁸R⁹, COOR⁸, CON(R⁸)SO₂R⁹, CON(R⁸)SO₂N(R⁸R⁹), NR⁸R⁹, NR⁸COOR⁹, OCOR⁸, NR⁸SO₂R⁹, SO₂NR⁸R⁹, SO₂R⁸, OCONR⁸R⁹, OCONR⁸R¹², N(R⁸)CON(R⁸R⁹), N(R⁸)COOR¹²; or C₁-C₄ alkyloxyC₁-C₄ alkyloxy; Het¹ can represents a 4 to 6 membered saturated ring containing one N atom, optionally being substituted with one or more substituents each independently selected from halo, C₁-C₄ alkyloxy, SO₂R⁸, C₁-C₄ alkylcarbonyl, CO(aryl), COHet², C₁-C₄ alkyloxycarbonyl, pyridinyl, CF₃, SO₂N(C₁-C₄ alkyl)₂, SO₂NH(C₁-C₄ alkyl), (C═O)NH(C₁-C₄ alkyl), (C═S)NH(C₁-C₄ alkyl), C₁-C₄ alkyl and C₁-C₄ alkyl substituted with one hydroxy; or Het¹ can represents a 4 to 6 membered saturated ring containing one O atom, substituted with one or more substituents each independently selected from halo, C₁-C₄ alkyloxy, CF₃, NH(C═O)(C₁-C₄ alkyl), (C═O)NH(C₁-C₄ alkyl) and C₁-C₄ alkyl; or Het represents a bicyclic 7 to 11 membered non-aromatic heterocycle containing one or two heteroatoms each independently selected from O, S and N, optionally substituted with one or more substituents each independently selected from halo, C₁-C₄ alkyloxy, SO₂R⁸, C₁-C₄ alkylcarbonyl, CO(aryl), COHet², C₁-C₄ alkyloxycarbonyl, pyridinyl, CF₃, SO₂N(C₁-C₄ alkyl)₂, SO₂NH(C₁-C₄ alkyl), (C═O)NH(C₁-C₄ alkyl), (C═S)NH(C₁-C₄ alkyl), C₁-C₄ alkyl and C₁-C₄ alkyl substituted with one hydroxy; Het² can represents a monocyclic 5 to 6 membered aromatic heterocycle containing one or more heteroatoms each independently selected from O, S and N; or a bicyclic 8 to 12 membered aromatic heterocycle containing one or more heteroatoms each independently selected from O, S and N; said Het² optionally being substituted with one or more substituents each independently selected from halo, C₁-C₄ alkyloxy, C₁-C₄ alkyl, OH, CN, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, COR⁸, NR⁸SO₂R⁹, SO₂NR⁸R⁹, SO₂R⁸, CONR⁸R⁹, CONRV², N(R⁸)CON(R⁸R⁹), N(R⁸)COOR¹²; Z can be C or N; R⁵ is present where Z is C, whereby R⁵ can be selected form hydrogen, CF₃ and halogen; R⁵ is absent where Z is N; or a pharmaceutically acceptable addition salt or a solvate thereof.

Examples of Compounds of Formula (B1) include:

| | Structure |
|---|---|
| P1 | 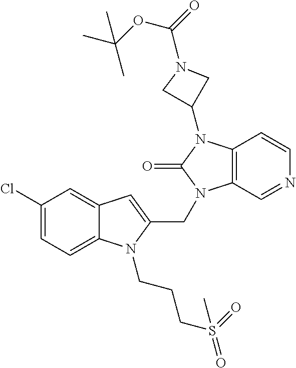 [SMILES: CC(C)(C)OC(=O)N1CC(n2c(=O)n(Cc3cc4cc(Cl)ccc4n3CCCS(C)(=O)=O)c3cnccc32)C1] |
| P2 | 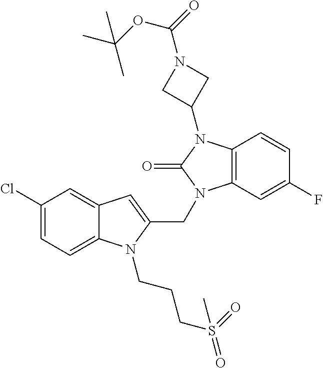 [SMILES: CC(C)(C)OC(=O)N1CC(n2c(=O)n(Cc3cc4cc(Cl)ccc4n3CCCS(C)(=O)=O)c3cc(F)ccc32)C1] |
| P3 | 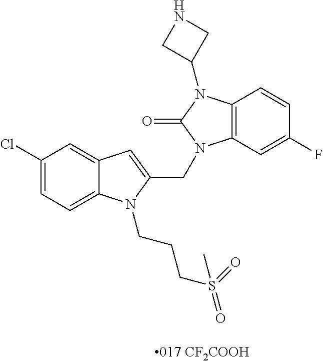 [SMILES: CS(=O)(=O)CCCn1c(Cn2c(=O)n(C3CNC3)c3ccc(F)cc32)cc2cc(Cl)ccc21] |
| P4 | 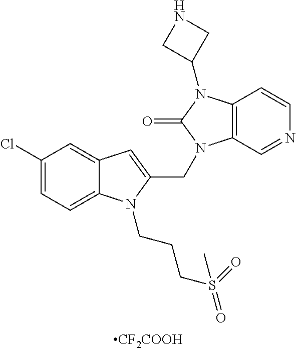 [SMILES: CS(=O)(=O)CCCn1c(Cn2c(=O)n(C3CNC3)c3ccncc32)cc2cc(Cl)ccc21] |
| P5 | 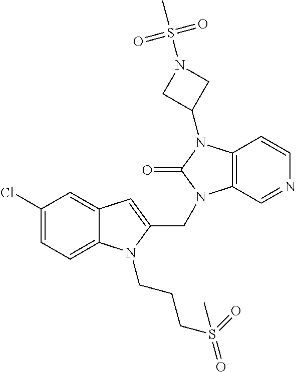 [SMILES: CS(=O)(=O)CCCn1c(Cn2c(=O)n(C3CN(S(C)(=O)=O)C3)c3ccncc32)cc2cc(Cl)ccc21] |
| P6 | 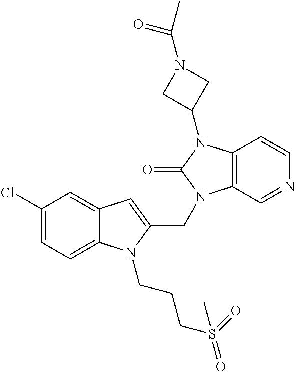 [SMILES: CC(=O)N1CC(n2c(=O)n(Cc3cc4cc(Cl)ccc4n3CCCS(C)(=O)=O)c3cnccc32)C1] |
| P7 | 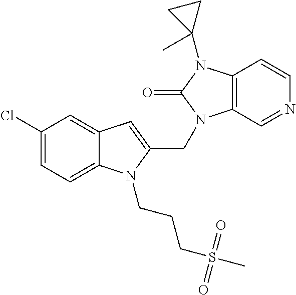 [SMILES: CC1(n2c(=O)n(Cc3cc4cc(Cl)ccc4n3CCCS(C)(=O)=O)c3cnccc32)CC1] |
| P8 | 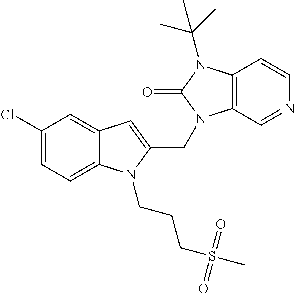 [SMILES: CC(C)(C)n1c(=O)n(Cc2cc3cc(Cl)ccc3n2CCCS(C)(=O)=O)c2cnccc21] |
| P9 | 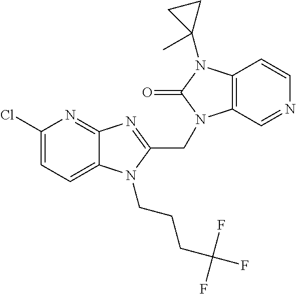 [SMILES: CC1(n2c(=O)n(Cc3nc4nc(Cl)ccc4n3CCCC(F)(F)F)c3cnccc32)CC1] |
| P10 | 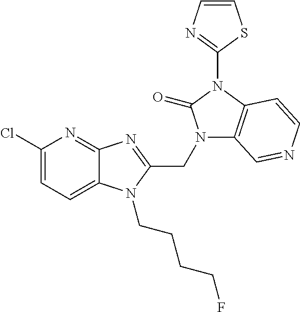 [SMILES: O=c1n(Cc2nc3nc(Cl)ccc3n2CCCCF)c2cnccc2n1-c1nccs1] |
| P11 | 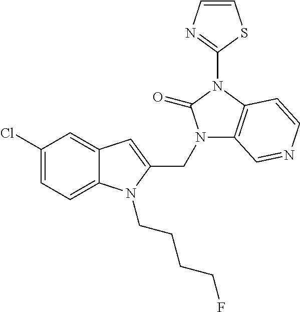 [SMILES: O=c1n(Cc2cc3cc(Cl)ccc3n2CCCCF)c2cnccc2n1-c1nccs1] |
| P12 | 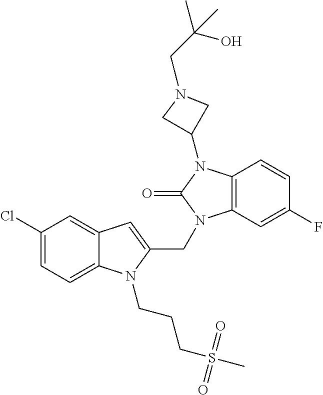 [SMILES: CC(C)(O)CN1CC(n2c(=O)n(Cc3cc4cc(Cl)ccc4n3CCCS(C)(=O)=O)c3cc(F)ccc32)C1] |
| P13 | 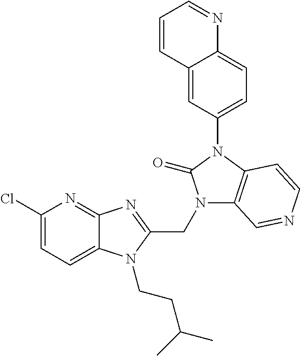 [SMILES: CC(C)CCn1c(Cn2c(=O)n(-c3ccc4ncccc4c3)c3ccncc32)nc2nc(Cl)ccc21] |
| P14 | 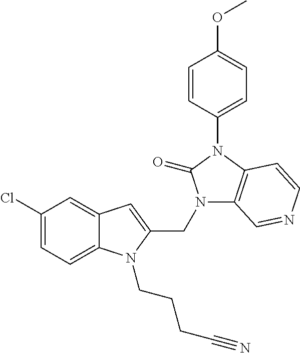 [SMILES: COc1ccc(-n2c(=O)n(Cc3cc4cc(Cl)ccc4n3CCCC#N)c3cnccc32)cc1] |
| P15 | 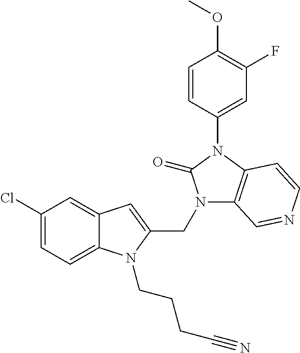 [SMILES: COc1ccc(-n2c(=O)n(Cc3cc4cc(Cl)ccc4n3CCCC#N)c3cnccc32)cc1F] |
| P16 | 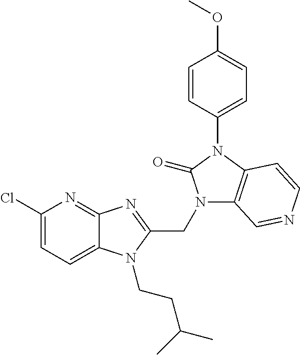 [SMILES: COc1ccc(-n2c(=O)n(Cc3nc4nc(Cl)ccc4n3CCC(C)C)c3cnccc32)cc1] |
| P17 | 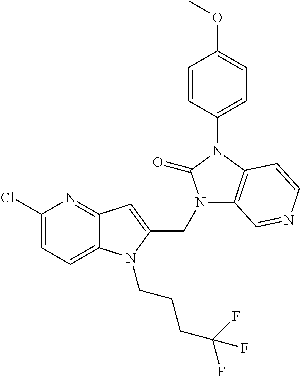 [SMILES: COc1ccc(-n2c(=O)n(Cc3cc4nc(Cl)ccc4n3CCCC(F)(F)F)c3cnccc32)cc1] |
| P18 | 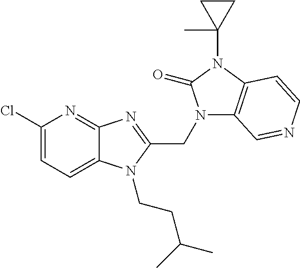 [SMILES: CC(C)CCn1c(Cn2c(=O)n(C3(C)CC3)c3ccncc32)nc2nc(Cl)ccc21] |
| P19 | 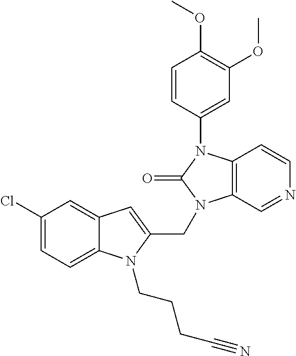 [SMILES: COc1ccc(-n2c(=O)n(Cc3cc4cc(Cl)ccc4n3CCCC#N)c3cnccc32)cc1OC] |
| P20 | 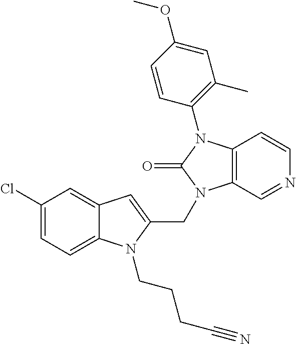 [SMILES: COc1ccc(-n2c(=O)n(Cc3cc4cc(Cl)ccc4n3CCCC#N)c3cnccc32)c(C)c1] |
| P21 | 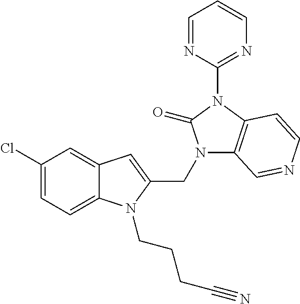 [SMILES: N#CCCCn1c(Cn2c(=O)n(-c3ncccn3)c3ccncc32)cc2cc(Cl)ccc21] |
| P22 | 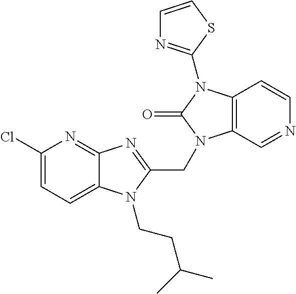 [SMILES: CC(C)CCn1c(Cn2c(=O)n(-c3nccs3)c3ccncc32)nc2nc(Cl)ccc21] |
| P23 | 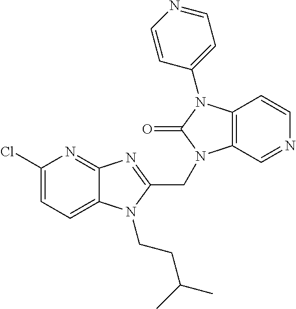 [SMILES: CC(C)CCn1c(Cn2c(=O)n(-c3ccncc3)c3ccncc32)nc2nc(Cl)ccc21] |
| P24 | 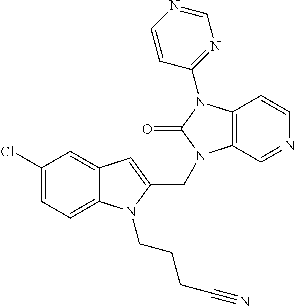 [SMILES: N#CCCCn1c(Cn2c(=O)n(-c3ccncn3)c3ccncc32)cc2cc(Cl)ccc21] |
| P25 | 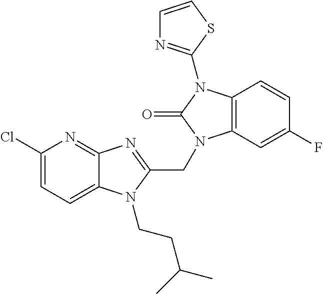 [SMILES: CC(C)CCn1c(Cn2c(=O)n(-c3nccs3)c3ccc(F)cc32)nc2nc(Cl)ccc21] |
| P26 | 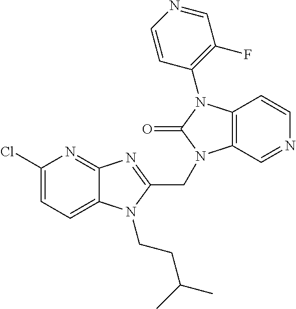 [SMILES: CC(C)CCn1c(Cn2c(=O)n(-c3ccncc3F)c3ccncc32)nc2nc(Cl)ccc21] |
| P27 | 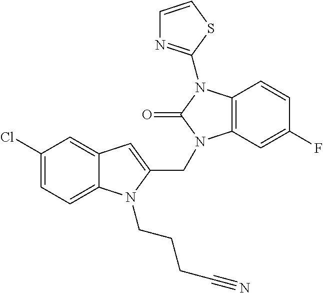 [SMILES: N#CCCCn1c(Cn2c(=O)n(-c3nccs3)c3ccc(F)cc32)cc2cc(Cl)ccc21] |
| P28 | 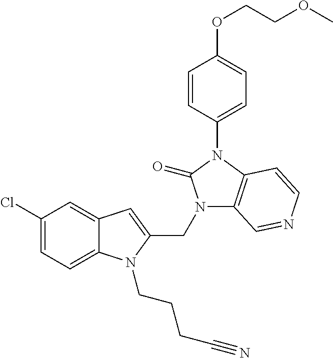 [SMILES: COCCOc1ccc(-n2c(=O)n(Cc3cc4cc(Cl)ccc4n3CCCC#N)c3cnccc32)cc1] |
| P29 | 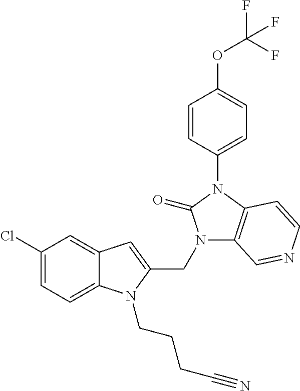 [SMILES: N#CCCCn1c(Cn2c(=O)n(-c3ccc(OC(F)(F)F)cc3)c3ccncc32)cc2cc(Cl)ccc21] |
| P30 | 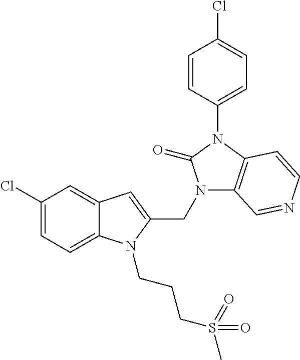 [SMILES: CS(=O)(=O)CCCn1c(Cn2c(=O)n(-c3ccc(Cl)cc3)c3ccncc32)cc2cc(Cl)ccc21] |
| P31 | 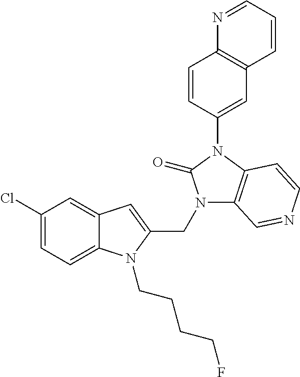 [SMILES: O=c1n(Cc2cc3cc(Cl)ccc3n2CCCCF)c2cnccc2n1-c1ccc2ncccc2c1] |
| P32 | 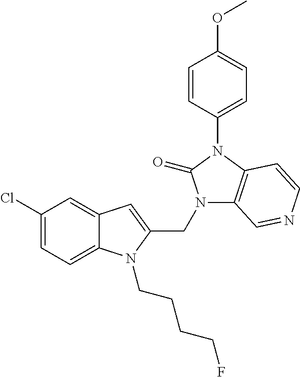 [SMILES: COc1ccc(-n2c(=O)n(Cc3cc4cc(Cl)ccc4n3CCCCF)c3cnccc32)cc1] |
| P33 | 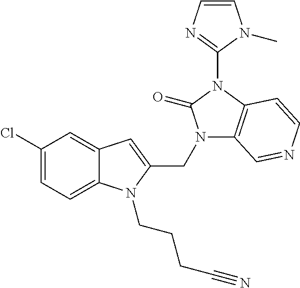 [SMILES: Cn1ccnc1-n1c(=O)n(Cc2cc3cc(Cl)ccc3n2CCCC#N)c2cnccc21] |
| P34 | 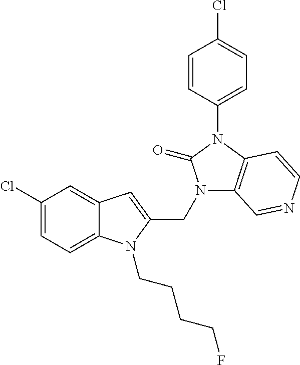 [SMILES: O=c1n(Cc2cc3cc(Cl)ccc3n2CCCCF)c2cnccc2n1-c1ccc(Cl)cc1] |
| P35 | 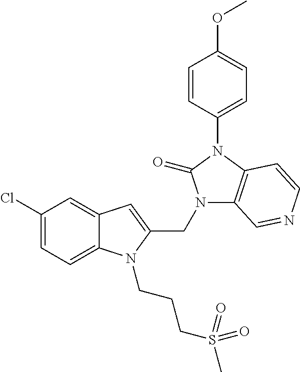 [SMILES: COc1ccc(-n2c(=O)n(Cc3cc4cc(Cl)ccc4n3CCCS(C)(=O)=O)c3cnccc32)cc1] |
| P36 | 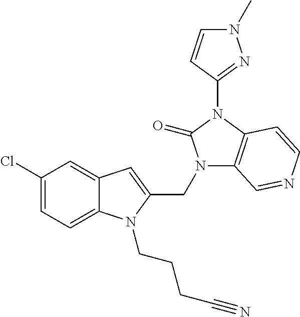 [SMILES: Cn1ccc(-n2c(=O)n(Cc3cc4cc(Cl)ccc4n3CCCC#N)c3cnccc32)n1] |
| P37 | 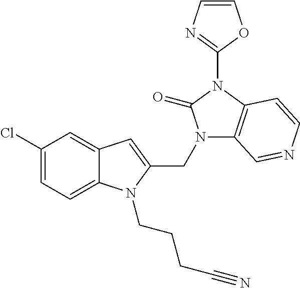 [SMILES: N#CCCCn1c(Cn2c(=O)n(-c3ncco3)c3ccncc32)cc2cc(Cl)ccc21] |
| P38 | 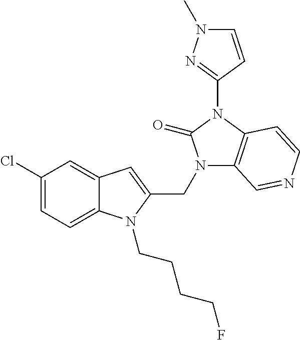 [SMILES: Cn1ccc(-n2c(=O)n(Cc3cc4cc(Cl)ccc4n3CCCCF)c3cnccc32)n1] |
| P39 | 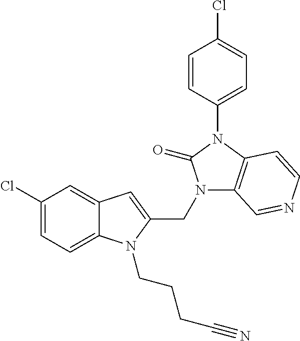 [SMILES: N#CCCCn1c(Cn2c(=O)n(-c3ccc(Cl)cc3)c3ccncc32)cc2cc(Cl)ccc21] |
| P40 | 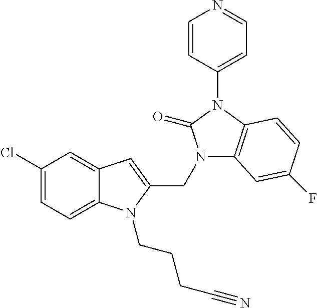 [SMILES: N#CCCCn1c(Cn2c(=O)n(-c3ccncc3)c3ccc(F)cc32)cc2cc(Cl)ccc21] |
| P41 | 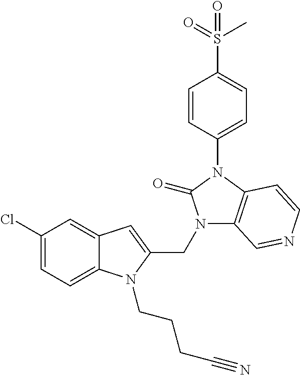 [SMILES: CS(=O)(=O)c1ccc(-n2c(=O)n(Cc3cc4cc(Cl)ccc4n3CCCC#N)c3cnccc32)cc1] |
| P42 | 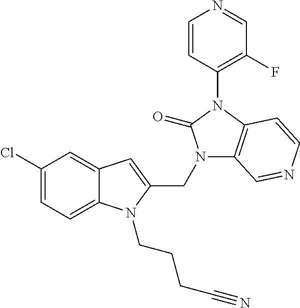 [SMILES: N#CCCCn1c(Cn2c(=O)n(-c3ccncc3F)c3ccncc32)cc2cc(Cl)ccc21] |
| P43 | 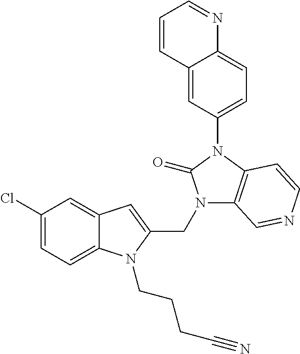 [SMILES: N#CCCCn1c(Cn2c(=O)n(-c3ccc4ncccc4c3)c3ccncc32)cc2cc(Cl)ccc21] |
| P44 | 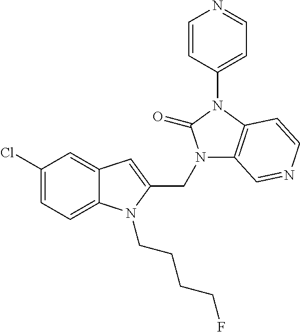 [SMILES: O=c1n(Cc2cc3cc(Cl)ccc3n2CCCCF)c2cnccc2n1-c1ccncc1] |
| P45 | 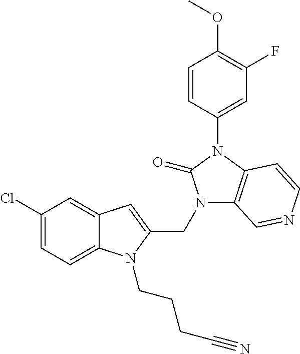 [SMILES: COc1ccc(-n2c(=O)n(Cc3cc4cc(Cl)ccc4n3CCCC#N)c3cnccc32)cc1F] |
| P46 | 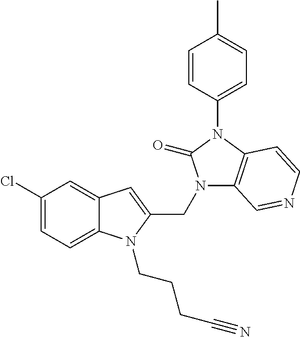 [SMILES: Cc1ccc(-n2c(=O)n(Cc3cc4cc(Cl)ccc4n3CCCC#N)c3cnccc32)cc1] |
| P47 | 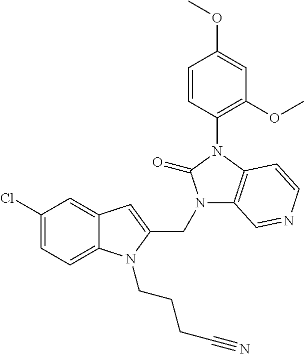 [SMILES: COc1ccc(-n2c(=O)n(Cc3cc4cc(Cl)ccc4n3CCCC#N)c3cnccc32)c(OC)c1] |
| P48 | 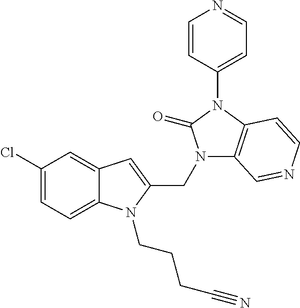 [SMILES: N#CCCCn1c(Cn2c(=O)n(-c3ccncc3)c3ccncc32)cc2cc(Cl)ccc21] |
| P49 | 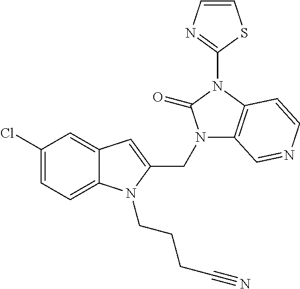 [SMILES: N#CCCCn1c(Cn2c(=O)n(-c3nccs3)c3ccncc32)cc2cc(Cl)ccc21] |
| P50 | 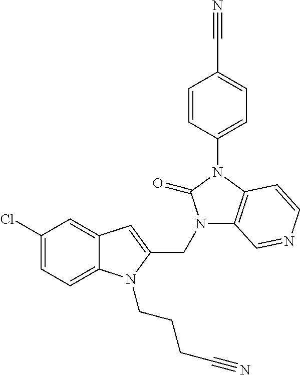 [SMILES: N#CCCCn1c(Cn2c(=O)n(-c3ccc(C#N)cc3)c3ccncc32)cc2cc(Cl)ccc21] |
| P51 | 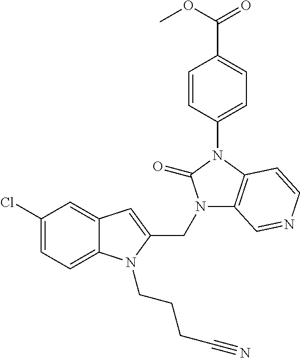 [SMILES: COC(=O)c1ccc(-n2c(=O)n(Cc3cc4cc(Cl)ccc4n3CCCC#N)c3cnccc32)cc1] |
| P52 | 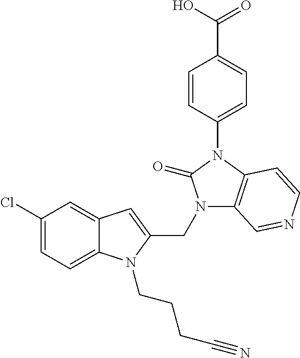 [SMILES: N#CCCCn1c(Cn2c(=O)n(-c3ccc(C(=O)O)cc3)c3ccncc32)cc2cc(Cl)ccc21] |
| P53 | 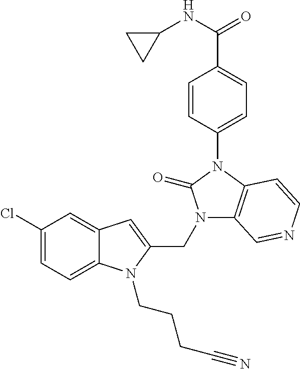 [SMILES: N#CCCCn1c(Cn2c(=O)n(-c3ccc(C(=O)NC4CC4)cc3)c3ccncc32)cc2cc(Cl)ccc21] |
| P54 | 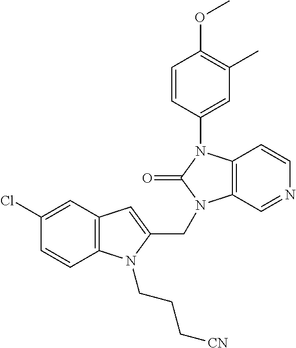 [SMILES: COc1ccc(-n2c(=O)n(Cc3cc4cc(Cl)ccc4n3CCCC#N)c3cnccc32)cc1C] |
| P55 | 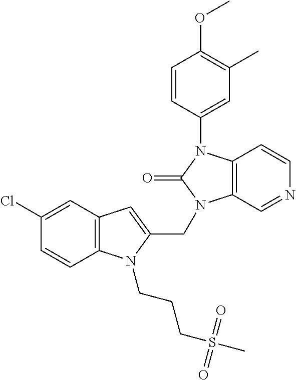 [SMILES: COc1ccc(-n2c(=O)n(Cc3cc4cc(Cl)ccc4n3CCCS(C)(=O)=O)c3cnccc32)cc1C] |
| P56 | 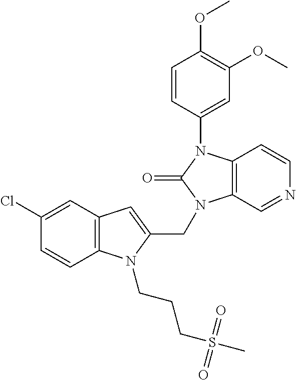 [SMILES: COc1ccc(-n2c(=O)n(Cc3cc4cc(Cl)ccc4n3CCCS(C)(=O)=O)c3cnccc32)cc1OC] |

### Compounds of Formula (B2)

Compounds of the general Formula (B2) are described in PCT Publication No. WO 2013/186332, published Dec. 19, 2013, which is hereby incorporated by reference in its entirety. Formula (B2) has the structure:

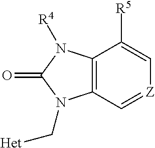

a tautomer or a stereoisomeric form thereof, wherein: Het can be a heterocycle having formula (a):

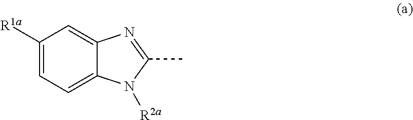

R^{1a} can be Br or Cl; R^{2a} can be —(CR^{8a}R^{9a})ₙ—R^{10a}; each R^{8a} and R^{9a} can be independently chosen from H, C₁-C₁₀ alkyl and C₃-C₇ cycloalkyl; or R^{8a} and R^{9a} can be taken together form a 4 to 6 membered aliphatic ring; wherein the 4 to 6 membered aliphatic ring optionally contains one or more heteroatoms selected from N, S and O; R^{10a} can be selected from H, C₁-C₆ alkyl, R¹¹, OH, CF₃, CHF₂, F, Cl, SO₂CH₃, SO₂C₃-C₇ cycloalkyl, NR^{8a}SO₂R^{8a}, SO₂NR^{8a}R^{9a}, NR^{8a}SO₂C₃-C₇ cycloalkyl, CN, NR^{8a}R^{9a}, COOH, COOR⁸a, CONR^{8a}R^{9a}, OCOC₁-C₆ alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from N, S and O; R¹¹ can be selected from C₁-C₆ alkyl, C₃-C₇ cycloalkyl, phenyl, pyridinyl and pyrazolyl; each substituted with one or more substituents each independently selected from CF₃, CH₃, CH₃, OCF₃ and halogen; n can be an integer having a value from 1 to 6; R⁵ can be selected from C₁-C₆ alkyl, C₁-C₆ alkyloxy, CN, CF₃ and halo; R⁴ can be selected from hydrogen, tert-butyl, C₃-C₇ cycloalkyl, CH(CH₃)(CF₃), C₂-C₁₀ alkenyl, CH₂CF₃, SO₂CH₃, —CH₂-p-fluorophenyl, aryl, Het¹, Het² and C₃-C₇ cycloalkyl substituted with one or more substituents selected from halo and C₁-C₄ alkyl; aryl can represents phenyl or naphthalenyl; said aryl optionally being substituted with one or more substituents each independently selected from halo, C₁-C₄ alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, COR^{8a}, NR^{8a}SO₂R^{9a}, SO₂NR^{8a}R^{9a}, SO₂R^{8a}, CONR^{8a}R^{9a}, CONR^{8a}R^{11a}, N(R^{8a})CON(R^{8a}R^{9a}), N(R^{8a})COOR^{11a}, and C₁-C₄ alkyl; Het¹ can represents a monocyclic 4 to 6 membered non-aromatic heterocycle containing one or two heteroatoms each independently selected from O, S and N; or a bicyclic 7 to 11 membered non-aromatic heterocycle containing one or two heteroatoms each independently selected from O, S and N; said Het¹ optionally being substituted with one or more substituents each independently selected from halo, C₁-C₄ alkyloxy, SO₂R^{8a} C₁-C₄ alkylcarbonyl, C₁-C₄ alkyloxycarbonyl, CO(aryl), COHet², pyridinyl, CF₃, SO₂N(C₁-C₄ alkyl)₂, SO₂NH(C₁-C₄ alkyl), NH(C═O)(C₁-C₄ alkyl, (C═O)NH(C₁-C₄ alkyl, (C═S)NH(C₁-C₄ alkyl), C₁-C₄ alkyl and C₁-C₄ alkyl substituted with one hydroxy; Het² can represents a monocyclic 5 to 6 membered aromatic heterocycle containing one or more heteroatoms each independently selected from O, S and N; or a bicyclic 8 to 12 membered aromatic heterocycle containing one or more heteroatoms each independently selected from O, S and N; said Het² optionally being substituted with one or more substituents each independently selected from halo, C₁-C₄ alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, COR^{8a}, NR^{8a}SO₂R^{9a}, SO₂NR^{8a}R^{9a}, SO₂R^{8a}, CONR^{8a}R^{9a}, CONR^{8a}R^{11a}, N(R^{8a})CON(R^{8a}R^{9a}), N(R^{8a})COOR^{11a} and C₁-C₄ alkyl; R^{1c} a can be selected from phenyl, pyridinyl and pyrazolyl; each optionally substituted with one or more substituents each independently selected from CF₃, CH₃, OCH₃, CF₃ and halogen; or R^{1c}a can be C₁-C₆ alkyl or C₃-C₇ cycloalkyl; each substituted with one or more substituents each independently selected from CF₃, CH₃, CH₃, CF₃ and halogen; Z can be CH or N; or a pharmaceutically acceptable addition salt or a solvate thereof

Examples of Compounds of Formula (B2) Include:

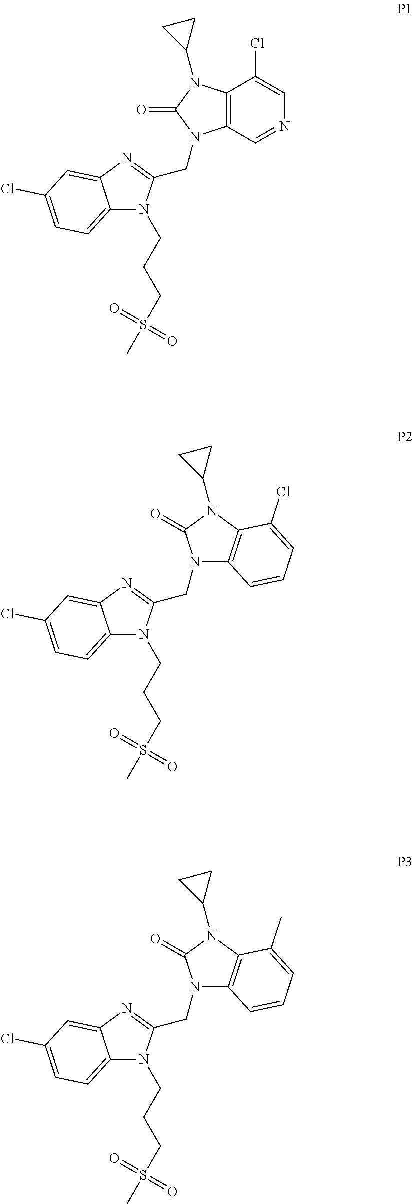 [SMILES: CS(=O)(=O)CCCn1c(Cn2c(=O)n(C3CC3)c3c(Cl)cncc32)nc2cc(Cl)ccc21 ; CS(=O)(=O)CCCn1c(Cn2c(=O)n(C3CC3)c3c(Cl)cccc32)nc2cc(Cl)ccc21 ; Cc1cccc2c1n(C1CC1)c(=O)n2Cc1nc2cc(Cl)ccc2n1CCCS(C)(=O)=O]

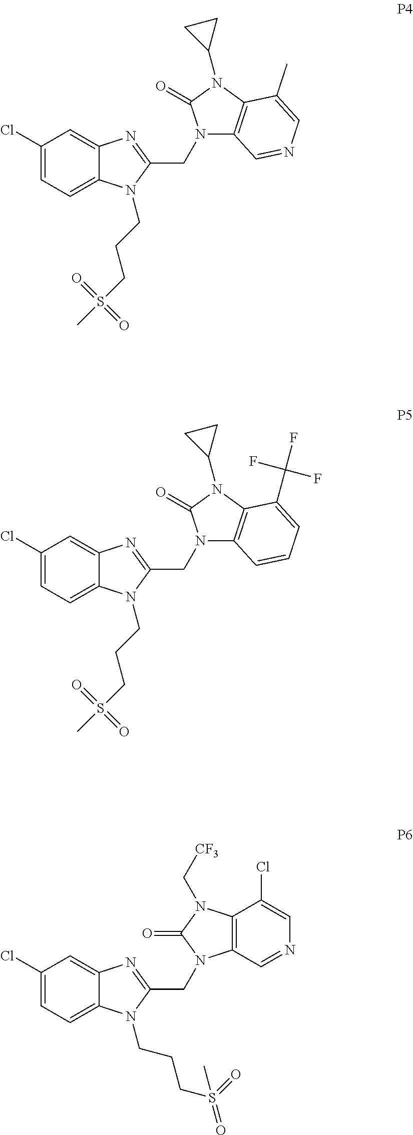 [SMILES: Cc1cncc2c1n(C1CC1)c(=O)n2Cc1nc2cc(Cl)ccc2n1CCCS(C)(=O)=O ; CS(=O)(=O)CCCn1c(Cn2c(=O)n(C3CC3)c3c(C(F)(F)F)cccc32)nc2cc(Cl)ccc21 ; CS(=O)(=O)CCCn1c(Cn2c(=O)n(CC(F)(F)F)c3c(Cl)cncc32)nc2cc(Cl)ccc21]

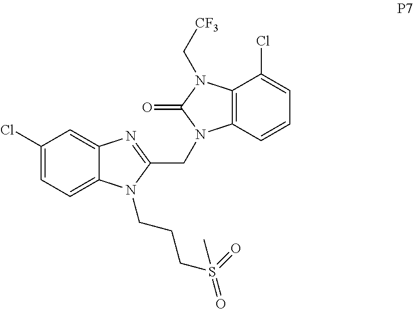 [SMILES: CS(=O)(=O)CCCn1c(Cn2c(=O)n(CC(F)(F)F)c3c(Cl)cccc32)nc2cc(Cl)ccc21]

### Compounds of Formula (B3)

Compounds of the general Formula (B3) are described in PCT Publication No. WO 2013/186335, published Dec. 19, 2013, which is hereby incorporated by reference in its entirety. Formula (B3) has the structure:

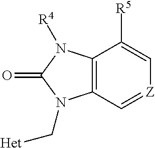

a tautomer or a stereoisomeric form thereof, wherein: Het can be a heterocycle having formula (b), (c), (d) or (e):

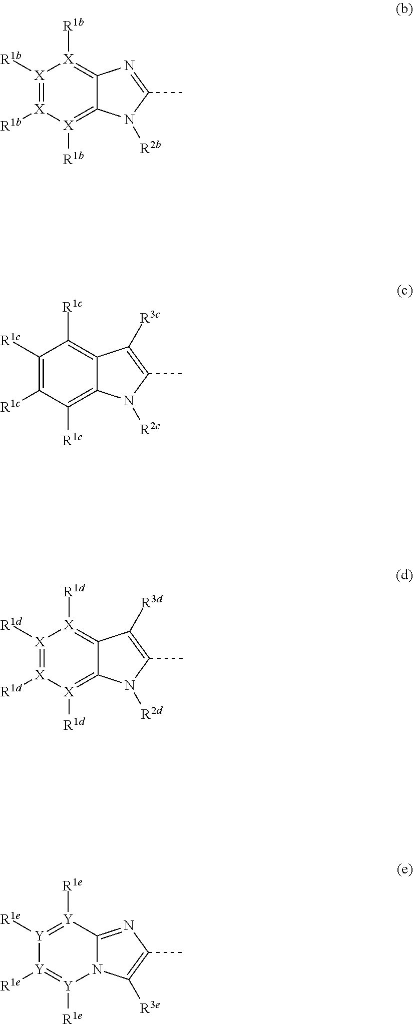

each X independently can be C or N; provided that at least one X is N; R^{1b} can be present when Het has formula (b) and X is C; each R^{1b} can be selected independently from H, halogen, C₁-C₆ alkyl, C₃-C₇ cycloalkyl, C₁-C₆ alkyloxy, N(R⁶)₂, CO(R⁷), CH₂NH₂, CH₂OH, CN, C(═NOH)NH₂, C(═NOCH₃)NH₂, C(═NH)NH₂, CF₃, CF₃, B(OH)₂ and B(O—C₁-C₆ alkyl)₂; R^{1b} can be absent when the X to which it is bound is N; R^{2b} can be —(CR⁸R⁹)ₘ—R^{10b}; each R⁶ can be independently selected from can be H, C₁-C₆ alkyl, COOCH₃ and CONHSO₂CH₃; each R⁷ can be independently selected from OH, C₁-C₆ alkyloxy, NH₂, NHSO₂N(C₁-C₆ alkyl)₂, NHSO₂NHCH₃, NHSO₂(C₁-C₆ alkyl), NHSO₂(C₃-C₇ cycloalkyl) and N(C₁-C₆-alkyl)₂; each R⁸ and R⁹ can be independently chosen from H, C₁-C₆ alkyl and C₃-C₇ cycloalkyl; or R⁸ and R⁹ can be taken together form a 4 to 6 membered aliphatic ring that optionally contains one or more heteroatoms selected from N, S and O; R^{10b} can be selected from H, R¹¹, OH, CN, F, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, COR⁸, O-Benzyl, NR⁸SO₂R⁹, SO₂NR⁸R⁹, SO₂R⁸, OCONR⁸R⁹, CONR⁸R¹², N(R⁸)CON(R⁸R⁹), N(R⁸)COOR¹² and a 4 to 6 membered saturated ring containing one oxygen atom; R¹¹ can be selected from C₁-C₆ alkyl, C₃-C₇ cycloalkyl, phenyl, pyridinyl and pyrazolyl; each optionally substituted with one or more substituents each independently selected from CF₃, CH₃, CH₃, CF₃ and halogen; R¹² can be selected from phenyl, pyridinyl and pyrazolyl; each optionally substituted with one or more substituents each independently selected from CF₃, CH₃, CH₃, CF₃ and halogen; or R¹² can be C₁-C₆ alkyl or C₃-C₇ cycloalkyl; each substituted with one or more substituents each independently selected from CF₃, CH₃, CH₃, CF₃ and halogen; m can be an integer from 2 to 6; R^{1c} can be present when Het has formula (c); each R^{1c} can be selected independently from H, halogen, C₁-C₆ alkyl, C₃-C₇ cycloalkyl, C₁-C₆ alkyloxy, N(R⁶)₂, CO(R^{7c}), CH₂NH₂, CH₂OH, CN, C(═NOH)NH₂, C(═NOCH₃)NH₂, C(═NH)NH₂, CF₃, CF₃, B(OH)₂ and B(O—C₁-C₆alkyl)₂; R^{1c} can be selected from H, halogen, C₁-C₆ alkyl, C₃-C₇ cycloalkyl, C₁-C₆ alkyloxy and CO(R^{7c}); R^{2c} can be —(CR⁸R⁹)ₘ—R^{10c}; R^{7c} can be selected from OH, O(C₁-C₆ alkyl), NH₂, NHSO₂N(C₁-C₆ alkyl)₂, NHSO₂NHCH₃, NHSO₂(C₁-C₆ alkyl), NHSO₂(C₃-C₇ cycloalkyl), N(C₁-C₆-alkyl)₂, NR⁸R⁹ and NR₉R^{10c}; R^{10c} can be selected from H, R¹¹, OH, CN, F, CF₂H, CF₃, C(═NOH)NH₂, CONR⁸R⁹, COOR⁸, CONR⁸SO₂R⁹, CON(R⁸)SO₂N(R⁸R⁹), NR⁸R⁹, NR⁸COOR⁹, COR⁸, NR⁸SO₂R⁹, SO₂NR⁸R⁹, SO₂R⁸ and a 4 to 6 membered saturated ring containing one oxygen atom; R^{1d} can be present when Het has formula (d) and X is C; each R^{1d} can be selected independently from H, OH, halogen, C₁-C₆ alkyl, C₃-C₇ cycloalkyl, C₁-C₆ alkyloxy, N(R⁶)₂, CO(R⁷), CH₂NH₂, CH₂OH, CN, C(═NOH)NH₂, C(═NOCH₃)NH₂, C(═NH)NH₂, CF₃, CF₃, B(OH)₂ and B(O—C₁-C₆ alkyl)₂; R^{1d} is absent when the X to which it is bound is N; R^{3d} can be selected from H, halogen, C₁-C₆ alkyl, C₃-C₇ cycloalkyl, C₁-C₆ alkyloxy, and CO(R⁷); R^{2d} can be —(CR⁸R⁹)ₘ—R^{10d}; R^{10d} can be selected from H, R¹¹, OH, CN, F, CF₂H, CF₃, CONR⁸R⁹, COOR⁸, CONR⁸SO₂R⁹, CON(R⁸)SO₂N(R⁸R⁹), NR⁸R⁹, NR⁸COOR⁹, OCOR⁸, NR⁸SO₂R⁹, SO₂NR⁸R⁹, SO₂R⁸ and a 4 to 6 membered saturated ring containing one oxygen atom; each Y independently can be C or N; R^{1e} can be present when Het has formula (e) and Y is C; each R^{1e} can be selected independently from H, halogen, C₁-C₆ alkyl, C₃-C₇ cycloalkyl, C₁-C₆ alkyloxy, N(R⁶)₂, CO(R⁷), CH₂NH₂, CH₂OH, CN, C(═NOH)NH₂, C(═NOCH₃)NH₂, C(═NH)NH₂, CF₃, CF₃, B(OH)₂ and B(O—C₁-C₆ alkyl)₂; R^{1e} is absent when the Y to which it is bound is N; R^{3e} can be selected from H, halogen, —(CR⁸R⁹)ₘ—R^{10e}, C≡C—CH₂—R^{10e}, C≡C—R^{10e} and C≡C—R^{10e}; R^{10e} can be selected from H, R¹¹, C₁-C₆ alkyloxy, OH, CN, F, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, COR⁸, NR⁸SO₂R⁹, SO₂NR⁸R⁹, SO₂R⁸ and a 4 to 6 membered saturated ring containing one oxygen atom; R⁵ can be selected from C₁-C₆ alkyl, C₁-C₆ alkyloxy, CN, CF₃ and halogen; R⁴ can be selected from hydrogen, C₃-C₇ cycloalkyl, tert-butyl, C₂-C₁₀ alkenyl, CH₂CF₃, CH(CH₃)(CF₃), SO₂CH₃, —CH₂-p-fluorophenyl, aryl, Het¹, Het² and C₃-C₇ cycloalkyl substituted with one or more substituents selected from halo and C₁-C₄ alkyl; aryl can represents phenyl or naphthalenyl; said aryl optionally being substituted with one or more substituents each independently selected from halo, C₁-C₄alkyloxy, C₁-C₄alkyl, OH, CN, CF₂H, CF₃, CONR⁸R⁹, COOR⁸, CON(R⁸)SO₂R⁹, CON(R⁸)SO₂N(R⁸R⁹), NR⁸R⁹, NR⁸COOR⁹, COR⁸, NR⁸SO₂R⁹, SO₂NR⁸R⁹, SO₂R⁸, CONR⁸R⁹, OCONR⁸R¹², N(R⁸)CON(R⁸R⁹) and N(R⁸)COOR¹²; Het¹ can represents a monocyclic 4 to 6 membered non-aromatic heterocycle containing one or two heteroatoms each independently selected from O, S and N; or a bicyclic 7 to 11 membered non-aromatic heterocycle containing one or two heteroatoms each independently selected from O, S and N; said Het¹ optionally being substituted with one or more substituents each independently selected from halo, C₁-C₄ alkyloxy, SO₂R, C₁-C₄ alkylcarbonyl, CO(aryl), COHet², C₁-C₄ alkyloxycarbonyl, pyridinyl, CF₃, SO₂N(C₁-C₄ alkyl)₂, SO₂NH(C₁-C₄ alkyl), NH(C═O)(C₁-C₄ alkyl, (C═O)NH(C₁-C₄ alkyl, (C═S)NH(C₁-C₄ alkyl) and C₁-C₄ alkyl; Het² can represents a monocyclic 5 to 6 membered aromatic heterocycle containing one or more heteroatoms each independently selected from O, S and N; or a bicyclic 8 to 12 membered aromatic heterocycle containing one or more heteroatoms each independently selected from O, S and N; said Het² optionally being substituted with one or more substituents each independently selected from halo, C₁-C₄ alkyloxy, C₁-C₄ alkyl, OH, CN, CF₂H, CF₃, CONRV, COOR⁸, CON(R⁸)SO₂R⁹, CON(R⁸)SO₂N(R⁸R⁹), NR⁸R⁹, NR⁸COOR⁹, COR⁸, NR⁸SO₂R⁹, SO₂NR⁸R⁹, SO₂R⁸, CONR⁸R⁹, CONR⁸R¹², N(R⁸)CON(R⁸R⁹) and N(R⁸)COOR¹²; Z can be CH or N; or a pharmaceutically acceptable addition salt or a solvate thereof.

Examples of Compounds of Formula (B3) include:

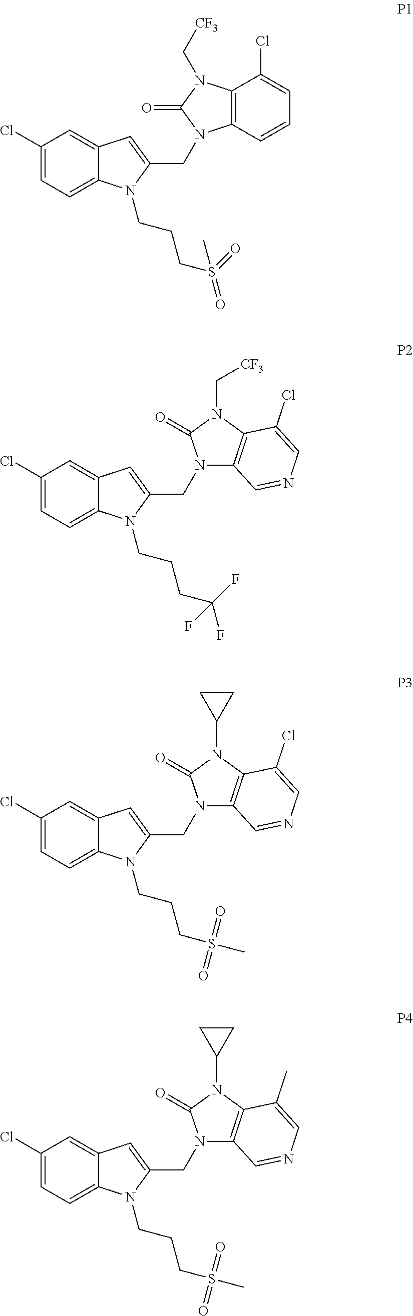 [SMILES: CS(=O)(=O)CCCn1c(Cn2c(=O)n(CC(F)(F)F)c3c(Cl)cccc32)cc2cc(Cl)ccc21 ; O=c1n(Cc2cc3cc(Cl)ccc3n2CCCC(F)(F)F)c2cncc(Cl)c2n1CC(F)(F)F ; CS(=O)(=O)CCCn1c(Cn2c(=O)n(C3CC3)c3c(Cl)cncc32)cc2cc(Cl)ccc21 ; Cc1cncc2c1n(C1CC1)c(=O)n2Cc1cc2cc(Cl)ccc2n1CCCS(C)(=O)=O]

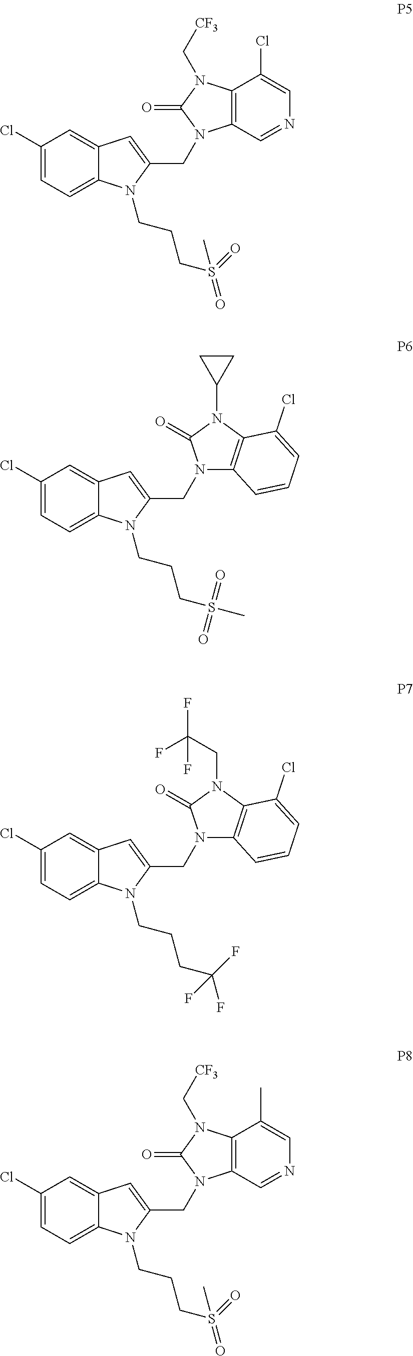 [SMILES: CS(=O)(=O)CCCn1c(Cn2c(=O)n(CC(F)(F)F)c3c(Cl)cncc32)cc2cc(Cl)ccc21 ; CS(=O)(=O)CCCn1c(Cn2c(=O)n(C3CC3)c3c(Cl)cccc32)cc2cc(Cl)ccc21 ; O=c1n(Cc2cc3cc(Cl)ccc3n2CCCC(F)(F)F)c2cccc(Cl)c2n1CC(F)(F)F ; Cc1cncc2c1n(CC(F)(F)F)c(=O)n2Cc1cc2cc(Cl)ccc2n1CCCS(C)(=O)=O]

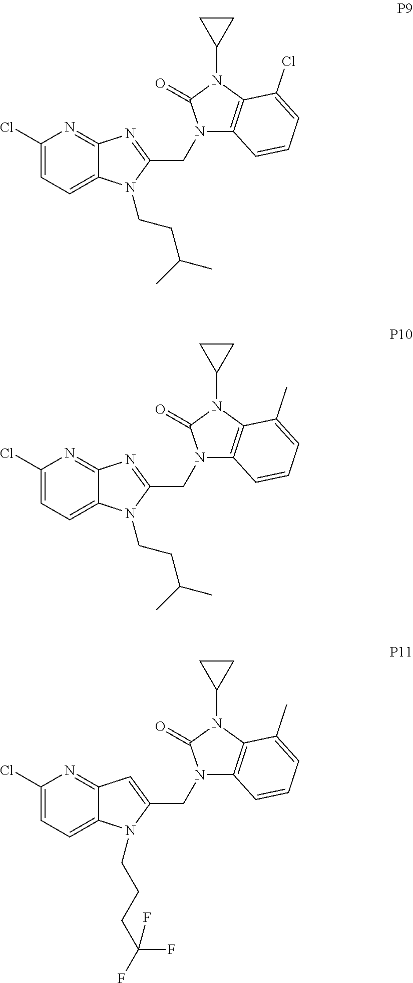 [SMILES: CC(C)CCn1c(Cn2c(=O)n(C3CC3)c3c(Cl)cccc32)nc2nc(Cl)ccc21 ; Cc1cccc2c1n(C1CC1)c(=O)n2Cc1nc2nc(Cl)ccc2n1CCC(C)C ; Cc1cccc2c1n(C1CC1)c(=O)n2Cc1cc2nc(Cl)ccc2n1CCCC(F)(F)F]

### Compounds of Formula (B4)

Compounds of the general Formula (B4) are described in PCT Publication No. WO 2013/186334, published Dec. 19, 2013, which is hereby incorporated by reference in its entirety. Formula (B4) has the structure:

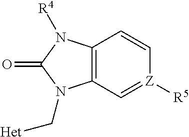

or a stereoisomeric form thereof, wherein: Het can be a heterocycle having formula (a):

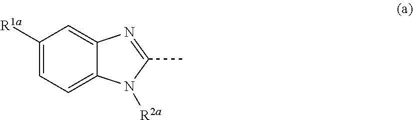

R^{1a} can be Br or Cl; R^{2a} can be —(CR^{8a}R^{9a})ₙ—R^{10a}; each R^{8a} and R^{9a} can be independently chosen from H, C₁-C₁₀ alkyl and C₃-C₇ cycloalkyl; or R^{8a} and R^{9a} can be taken together form a 4 to 6 membered aliphatic ring; wherein the 4 to 6 membered aliphatic ring optionally contains one or more heteroatoms selected N, S and O; R^{10a} can be selected from H, C₁-C₆ alkyl, R¹¹, OH, CF₃, CHF₂, F, Cl, SO₂CH₃, SO₂C₃-C₇ cycloalkyl, NR^{8a}SO₂R^{8a}, SO₂NR^{8a}R^{9a}, NR^{8a}SO₂C₃-C₇ cycloalkyl, CN, NR^{8a}R^{9a}, COOH, COOR^{8a}, CONR^{8a}R^{9a}, OCOC₁-C₆ alkyl, CONR^{8a}SO₂R^{9a}, CONR^{8a}SO₂NR^{8a}R^{9a}, a 4 to 6 membered aliphatic ring and a 5 to 6 membered aromatic ring; wherein the aliphatic or aromatic ring optionally contains one or more heteroatoms selected from N, S and O; R¹¹ can be selected from C₁-C₆ alkyl, C₃-C₇ cycloalkyl, phenyl, pyridinyl and pyrazolyl; each substituted with one or more substituents each independently selected from CF₃, CH₃, CH₃, OCF₃ and halogen; n can be an integer having a value from 1 to 6; R⁴ can be selected from tert-butyl, CH(CH₃)(CF₃), aryl, Het¹, Het² and C₃-C₇ cycloalkyl substituted with one or more substituents selected from halo and C₁-C₄ alkyl; aryl represents phenyl or naphthalenyl; said aryl optionally being substituted with one or more substituents each independently selected from halo, C₁-C₄ alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, COR^{8a}, NR^{8a}SO₂R^{9a}, SO₂NR^{8a}R^{9a}, SO₂R^{8a}, OCONR^{8a}R^{9a}, CONR^{8a}R^{11b}, N(R^{8a})CON(R^{8a}R^{9a}), N(R^{8a})COO—R^{11b}, and C₁-C₄ alkyl; Het¹ can represents a monocyclic 4 to 6 membered non-aromatic heterocycle containing one or two heteroatoms each independently selected from O, S and N; or a bicyclic 7 to 11 non-aromatic heterocycle containing one or two heteroatoms each independently selected from O, S and N; said Het¹ optionally being substituted with one or more substituents each independently selected from halo, C₁-C₄ alkyloxy, SO₂R^{8a} C₁-C₄ alkylcarbonyl, C₁-C₄ alkyloxycarbonyl, CO(aryl), COHet², pyridinyl, CF₃, SO₂N(C₁-C₄ alkyl)₂, SO₂NH(C₁-C₄ alkyl), NH(C═O)(C₁-C₄ alkyl, (C═O)NH(C₁-C₄ alkyl, (C═S)NH(C₁-C₄ alkyl), C₁-C₄ alkyl and C₁-C₄ alkyl substituted with one hydroxy; Het² can represents a monocyclic 5 to 6 membered aromatic heterocycle containing one or more heteroatoms each independently selected from O, S and N; or a bicyclic 8 to 12 membered aromatic heterocycle containing one or more heteroatoms each independently selected from O, S and N; said Het² optionally being substituted with one or more substituents each independently selected from halo, C₁-C₄ alkyloxy, OH, CN, CF₂H, CF₃, CONR^{8a}R^{9a}, COOR^{8a}, CON(R^{8a})SO₂R^{9a}, CON(R^{8a})SO₂N(R^{8a}R^{9a}), NR^{8a}R^{9a}, NR^{8a}COOR^{9a}, COR^{8a}, NR^{8a}SO₂R^{9a}, SO₂NR^{8a}R^{9a}, SO₂R^{8a}, OCONR^{8a}R^{9a}, CONR^{8a}R^{11b}, N(R^{8a})CON(R^{8a}R^{9a}), N(R^{8a})COOR^{11b} and C₁-C₄ alkyl; R^{11b} can be selected from phenyl, pyridinyl and pyrazolyl; each optionally substituted with one or more substituents each independently selected from CF₃, CH₃, CH₃, CF₃ and halogen; or R^{11b} can be C₁-C₆ alkyl or C₃-C₇ cycloalkyl; each substituted with one or more substituents each independently selected from CF₃, CH₃, CH₃, CF₃ and halogen; Z can be C or N; R⁵ is present where Z is C, whereby R⁵ can be selected from hydrogen, CF₃ and halogen; R⁵ is absent where Z is N; or a pharmaceutically acceptable addition salt or a solvate thereof.

Examples of Compounds of Formula (B4) include:

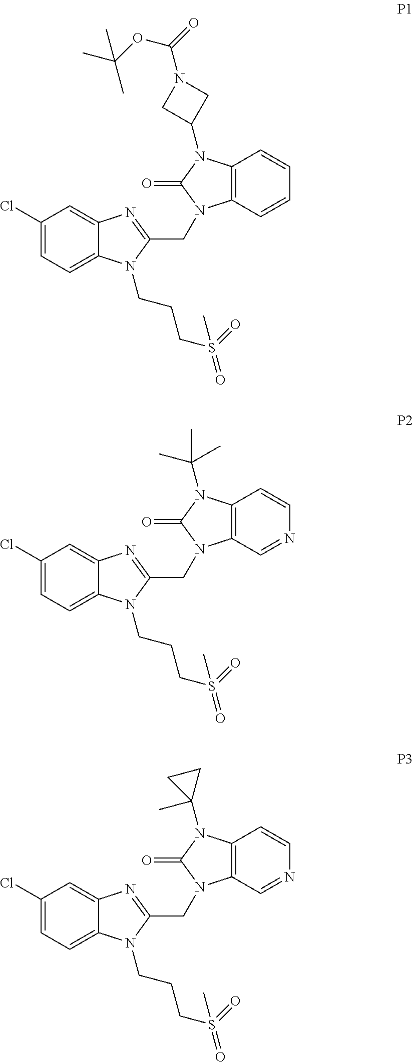 [SMILES: CC(C)(C)OC(=O)N1CC(n2c(=O)n(Cc3nc4cc(Cl)ccc4n3CCCS(C)(=O)=O)c3ccccc32)C1 ; CC(C)(C)n1c(=O)n(Cc2nc3cc(Cl)ccc3n2CCCS(C)(=O)=O)c2cnccc21 ; CC1(n2c(=O)n(Cc3nc4cc(Cl)ccc4n3CCCS(C)(=O)=O)c3cnccc32)CC1]

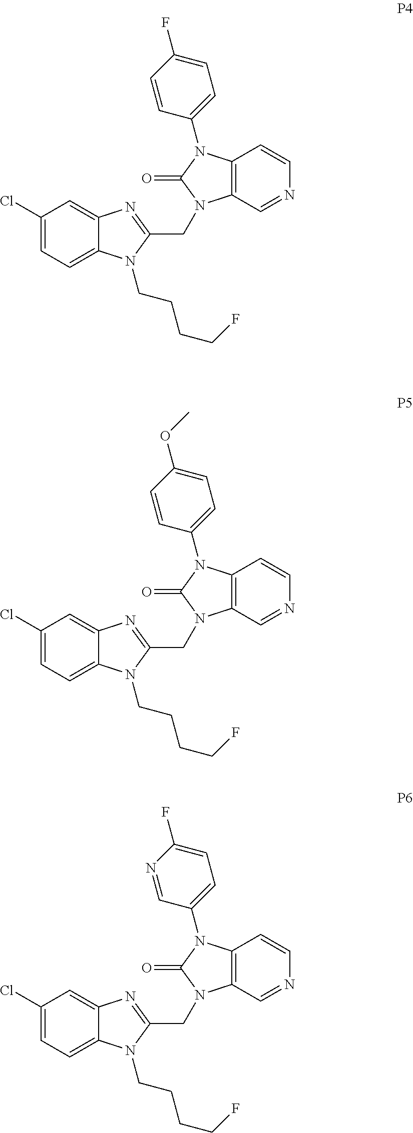 [SMILES: O=c1n(Cc2nc3cc(Cl)ccc3n2CCCCF)c2cnccc2n1-c1ccc(F)cc1 ; COc1ccc(-n2c(=O)n(Cc3nc4cc(Cl)ccc4n3CCCCF)c3cnccc32)cc1 ; O=c1n(Cc2nc3cc(Cl)ccc3n2CCCCF)c2cnccc2n1-c1ccc(F)nc1]

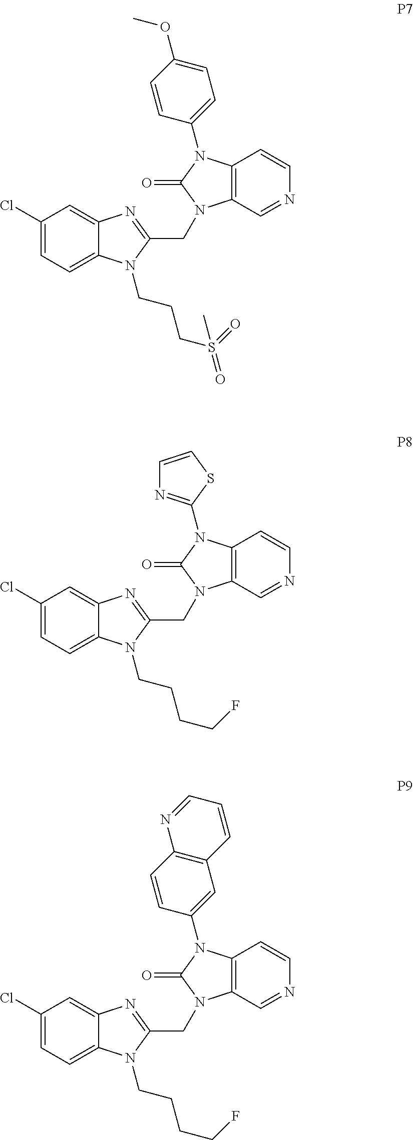 [SMILES: COc1ccc(-n2c(=O)n(Cc3nc4cc(Cl)ccc4n3CCCS(C)(=O)=O)c3cnccc32)cc1 ; O=c1n(Cc2nc3cc(Cl)ccc3n2CCCCF)c2cnccc2n1-c1nccs1 ; O=c1n(Cc2nc3cc(Cl)ccc3n2CCCCF)c2cnccc2n1-c1ccc2ncccc2c1]

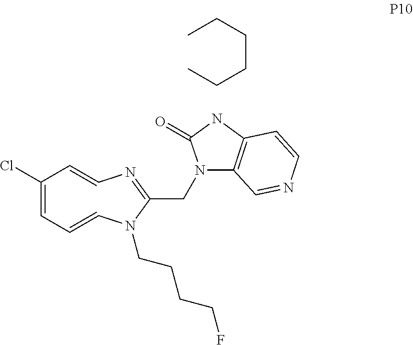

### Compounds of Formula (B5)

Compounds of the general Formula (B5) are described in PCT Publication No. WO 2012/080447, published Jun. 21, 2012, which is hereby incorporated by reference in its entirety. Formula (B5) has the structure:

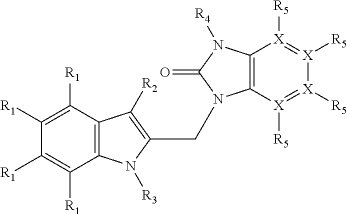

or a prodrug, N-oxide, addition salt, quaternary amine, metal complex, or a stereochemically isomeric form thereof, wherein: each X independently can be C or N; R₁ can be selected from H, halogen, C₁-C₆ alkyl, C₃-C₇ cycloalkyl, C₁-C₆ alkoxy, N(R₆)₂, CO(R₇), CH₂NH₂, CH₂OH, CN, C(═NOH)NH₂, C(═NOCH₃)NH₂, C(═NH)NH₂, CF₃, CF₃, and B(OH)₂; B(O—C₁-C₆ alkyl)₂; R₂ can be selected from H, halogen, C₁-C₆ alkyl, C₃-C₇ cycloalkyl, C₁-C₆ alkoxy, and CO(R₇); R₃ can be —(CR₈R₉)ₙ—R₁₀; R₄ can be selected from H, C₁-C₁₀ alkyl, C₃-C₇ cycloalkyl, C₂-C₁₀ alkenyl, SO₂—R₈, CH₂CF₃, SO₂CH₃ or a 4 to 6 membered saturated ring containing an oxygen atom; R₅ is present where X is C, and can be selected from H, C₁-C₆ alkyl, C₃-C₇ cycloalkyl, C₁-C₆ alkoxy, CO(R₇), CF₃ and halogen; R₅ is absent where X is N; R₆ can be selected from H, C₁-C₆ alkyl, COOCH₃, and CONHSO₂CH₃; R₇ can be selected from OH, O(C₁-C₆ alkyl), NH₂, NHSO₂N(C₁-C₆ alkyl)₂, NHSO₂NCH₃, NHSO₂(C₁-C₆ alkyl), NHSO₂(C₃-C₇ cycloalkyl), and N(C₁-C₆ alkyl)₂, NR₈R₉, NR₉R₁₀; n can be an integer from 2 to 6; R₈ and R₉ can be each independently chosen from H, C₁-C₁₀ alkyl, C₃-C₇ cycloalkyl or R₈ and R₉ can be taken together form a 4 to 6 membered aliphatic ring that optionally contains one or more heteroatoms selected from N, S, O; R₁₀ can be selected from H, C₁-C₆ alkyl, OH, CN, F, CF₂H, CF₃, C(═NOH)NH₂, CONR₈R₉, COOR₈, CONR₈SO₂R₉, CON(R₈)SO₂N(R₈R₉), NR₈R₉, NR₈COOR₉, COR₈, NR₈SO₂R₉, SO₂NR₈R₉, SO₂NR₈ or a 4 to 6 membered saturated ring containing an oxygen atom.

Examples of Compounds of Formula (B5) include:

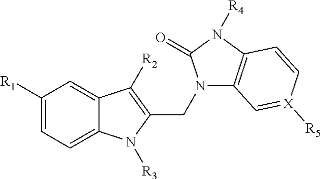

| | R₁ | R₂ | R₃ | R₄ | X—R₅ |
|---|---|---|---|---|---|
| P20 | F | H | 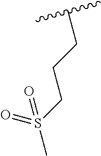 [SMILES: *CCCS(C)(=O)=O] | 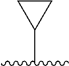 [SMILES: *C1CC1] | N |
| P21 | Br | H | 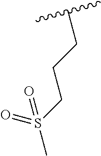 [SMILES: *CCCS(C)(=O)=O] | 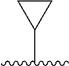 [SMILES: *C1CC1] | C—H |
| P22 | F | H | 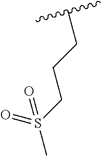 [SMILES: *CCCS(C)(=O)=O] | 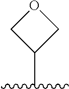 [SMILES: *C1COC1] | N |
| P23 | CF₃ | H | 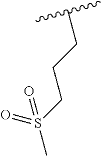 [SMILES: *CCCS(C)(=O)=O] | 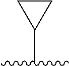 [SMILES: *C1CC1] | N |
| P24 | H | H | 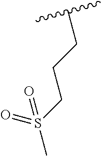 [SMILES: *CCCS(C)(=O)=O] | 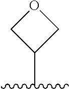 [SMILES: *C1COC1] | N |
| P25 | H | H | 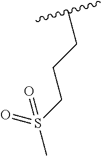 [SMILES: *CCCS(C)(=O)=O] | 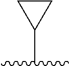 [SMILES: *C1CC1] | N |
| P26 | F | H | 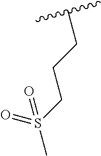 [SMILES: *CCCS(C)(=O)=O] | 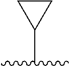 [SMILES: *C1CC1] | C—H |
| P27 | F | H | 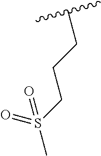 [SMILES: *CCCS(C)(=O)=O] | 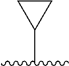 [SMILES: *C1CC1] | C—F |
| P28 | H | H | 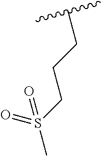 [SMILES: *CCCS(C)(=O)=O] | 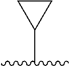 [SMILES: *C1CC1] | C—H |
| P29 | F | H | 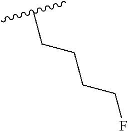 [SMILES: *CCCCF] | 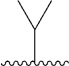 [SMILES: *C(C)C] | N |
| P30 | Cl | H | 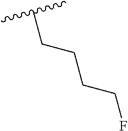 [SMILES: *CCCCF] | 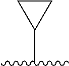 [SMILES: *C1CC1] | N |
| P31 | Br | H | 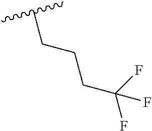 [SMILES: *CCCC(F)(F)F] | 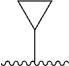 [SMILES: *C1CC1] | C—H |
| P32 | Br | H | 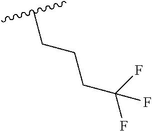 [SMILES: *CCCC(F)(F)F] | 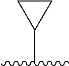 [SMILES: *C1CC1] | N |
| P33 | Cl | H | 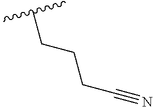 [SMILES: *CCCC#N] | 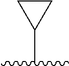 [SMILES: *C1CC1] | N |
| P34 | F | H | 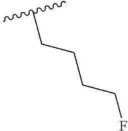 [SMILES: *CCCCF] | 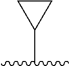 [SMILES: *C1CC1] | C—H |
| P35 | Cl | H | 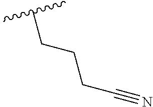 [SMILES: *CCCC#N] | 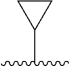 [SMILES: *C1CC1] | C—H |
| P36 | Cl | H | 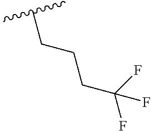 [SMILES: *CCCC(F)(F)F] | 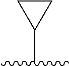 [SMILES: *C1CC1] | N |
| P37 | Cl | H | 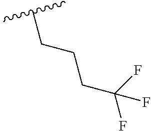 [SMILES: *CCCC(F)(F)F] | 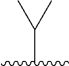 [SMILES: *C(C)C] | C—F |
| P38 | Cl | H | 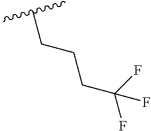 [SMILES: *CCCC(F)(F)F] | 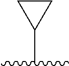 [SMILES: *C1CC1] | C—H |
| P39 | Cl | H | 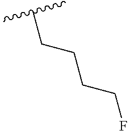 [SMILES: *CCCCF] | 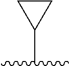 [SMILES: *C1CC1] | C—F |
| P40 | Cl | H | 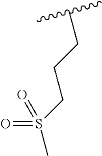 [SMILES: *CCCS(C)(=O)=O] | 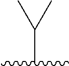 [SMILES: *C(C)C] | C—H |
| P41 | F | H | 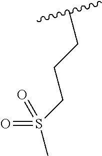 [SMILES: *CCCS(C)(=O)=O] | 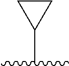 [SMILES: *C1CC1] | C—OCH₃ |
| P42 | Cl | H | 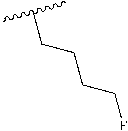 [SMILES: *CCCCF] | 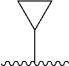 [SMILES: *C1CC1] | C—H |
| P43 | CN | H | 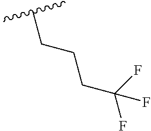 [SMILES: *CCCC(F)(F)F] | 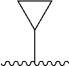 [SMILES: *C1CC1] | N |
| P44 | Cl | H | 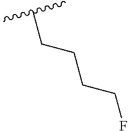 [SMILES: *CCCCF] | 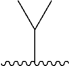 [SMILES: *C(C)C] | C—CONHMe |
| P45 | Br | H | 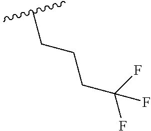 [SMILES: *CCCC(F)(F)F] | 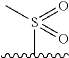 [SMILES: *S(C)(=O)=O] | N |
| P46 | H | H | 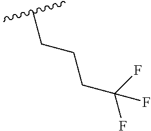 [SMILES: *CCCC(F)(F)F] | 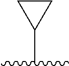 [SMILES: *C1CC1] | N |
| P47 | H | H | 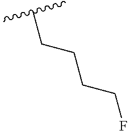 [SMILES: *CCCCF] | 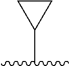 [SMILES: *C1CC1] | N |
| P48 | Cl | H | 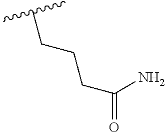 [SMILES: *CCCC(N)=O] | 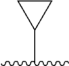 [SMILES: *C1CC1] | N |
| P49 | Cl | H | 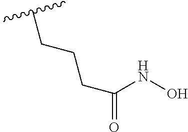 [SMILES: *CCCC(=O)NO] | 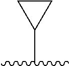 [SMILES: *C1CC1] | N |
| P50 | Cl | H | 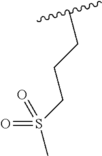 [SMILES: *CCCS(C)(=O)=O] | 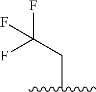 [SMILES: *CC(F)(F)F] | C—F |
| P51 | Cl | H | 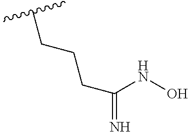 [SMILES: *CCCC(=N)NO] | 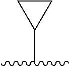 [SMILES: *C1CC1] | N |
| P52 | Cl | H | 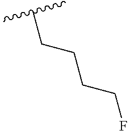 [SMILES: *CCCCF] | 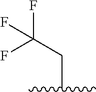 [SMILES: *CC(F)(F)F] | N |
| P53 | Cl | COOH | 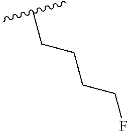 [SMILES: *CCCCF] | 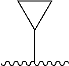 [SMILES: *C1CC1] | N |
| P54 | Cl | CONH₂ | 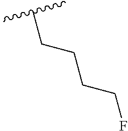 [SMILES: *CCCCF] | 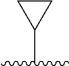 [SMILES: *C1CC1] | N |
| P55 | Cl | H | 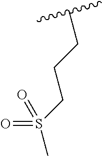 [SMILES: *CCCS(C)(=O)=O] | 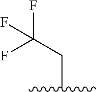 [SMILES: *CC(F)(F)F] | N |
| P57 | Cl | H | 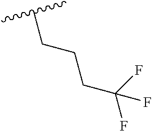 [SMILES: *CCCC(F)(F)F] | 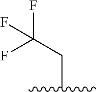 [SMILES: *CC(F)(F)F] | N |
| P58 | Cl | H | 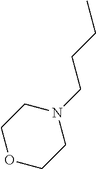 | 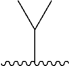 [SMILES: *C(C)C] | N |
| P59 | Cl | COOH | 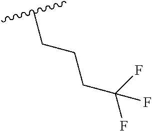 [SMILES: *CCCC(F)(F)F] | 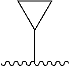 [SMILES: *C1CC1] | N |
| P60 | Cl | COOH | 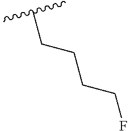 [SMILES: *CCCCF] | 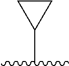 [SMILES: *C1CC1] | C—F |
| P61 | Cl | CONH₂ | 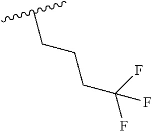 [SMILES: *CCCC(F)(F)F] | 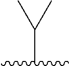 [SMILES: *C(C)C] | N |
| P62 | Cl | H | 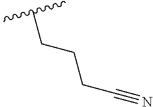 [SMILES: *CCCC#N] | 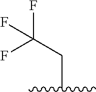 [SMILES: *CC(F)(F)F] | N |
| P63 | Cl | CONH₂ | 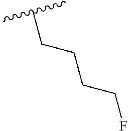 [SMILES: *CCCCF] | 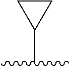 [SMILES: *C1CC1] | C—F |
| P64 | Cl | CONHMe | 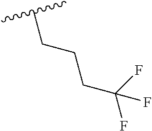 [SMILES: *CCCC(F)(F)F] | 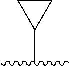 [SMILES: *C1CC1] | N |
| P65 | Cl | CONH₂ | 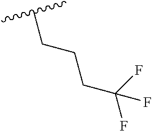 [SMILES: *CCCC(F)(F)F] | 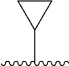 [SMILES: *C1CC1] | C—F |
| P66 | Cl | H | 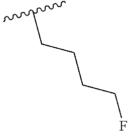 [SMILES: *CCCCF] | 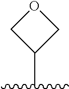 [SMILES: *C1COC1] | N |
| P67 | Cl | H | 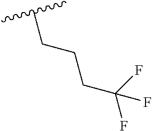 [SMILES: *CCCC(F)(F)F] | 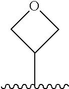 [SMILES: *C1COC1] | N |
| P68 | Cl | CONH cyclopropyl | 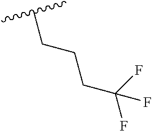 [SMILES: *CCCC(F)(F)F] | 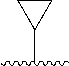 [SMILES: *C1CC1] | N |
| P69 | Cl | CONHMe | 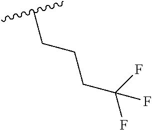 [SMILES: *CCCC(F)(F)F] | 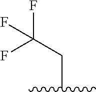 [SMILES: *CC(F)(F)F] | N |
| P70 | Cl | CONH₂ | 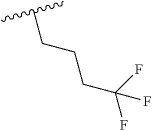 [SMILES: *CCCC(F)(F)F] | 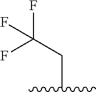 [SMILES: *CC(F)(F)F] | N |
| P71 | CN | H | 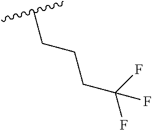 [SMILES: *CCCC(F)(F)F] | 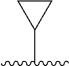 [SMILES: *C1CC1] | C—H |
| P72 | Cl | H | 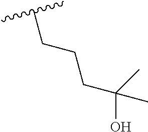 [SMILES: *CCCC(C)(C)O] | 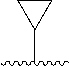 [SMILES: *C1CC1] | N |
| P73 | Cl | H | 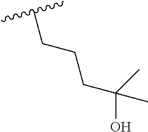 [SMILES: *CCCC(C)(C)O] | 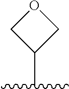 [SMILES: *C1COC1] | N |
| P74 | Cl | H | 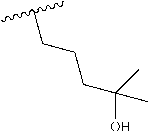 [SMILES: *CCCC(C)(C)O] | 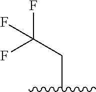 [SMILES: *CC(F)(F)F] | N |
| P75 | Cl | H | 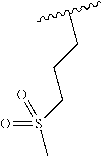 [SMILES: *CCCS(C)(=O)=O] | 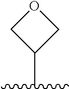 [SMILES: *C1COC1] | C—F |
| P79 | Cl | H | 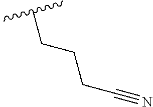 [SMILES: *CCCC#N] | 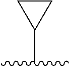 [SMILES: *C1CC1] | C—F |
| P80 | Cl | H | 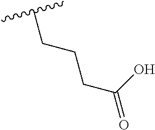 [SMILES: *CCCC(=O)O] | 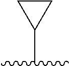 [SMILES: *C1CC1] | N |
| P81 | Cl | H | 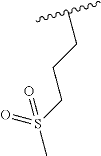 [SMILES: *CCCS(C)(=O)=O] | 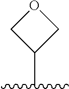 [SMILES: *C1COC1] | C—H |

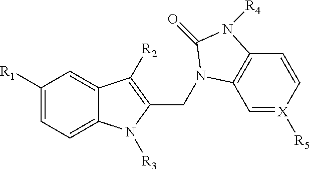

| | R₁ | R₂ | R₃ | R₄ | X—R₅ |
|---|---|---|---|---|---|
| P82 P83 P84 | Cl | CONH₂ | 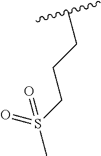 [SMILES: *CCCS(C)(=O)=O] | 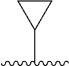 [SMILES: *C1CC1] | N CH CF |
| P85 P86 P87 | Cl | CONH₂ | 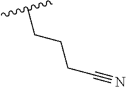 [SMILES: *CCCC#N] | 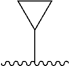 [SMILES: *C1CC1] | N CH CF |
| P88 P89 P90 | Cl | CONH₂ | 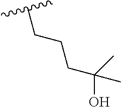 [SMILES: *CCCC(C)(C)O] | 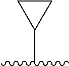 [SMILES: *C1CC1] | N CH CF |
| P91 P92 P93 | Cl | CONH₂ | 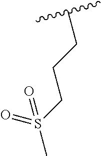 [SMILES: *CCCS(C)(=O)=O] | 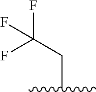 [SMILES: *CC(F)(F)F] | N CH CF |
| P94 P95 P96 | Cl | CONH₂ | 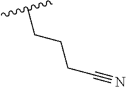 [SMILES: *CCCC#N] | 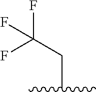 [SMILES: *CC(F)(F)F] | N CH CF |
| P97 P98 P99 | Cl | CONH₂ | 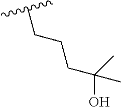 [SMILES: *CCCC(C)(C)O] | 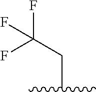 [SMILES: *CC(F)(F)F] | N CH CF |
| P100 P101 P102 | Cl | CONH₂ | 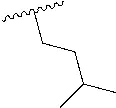 [SMILES: *CCC(C)C] | 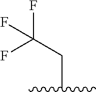 [SMILES: *CC(F)(F)F] | N CH CF |
| P103 P104 P105 | Cl | CONH₂ | 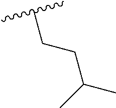 [SMILES: *CCC(C)C] | 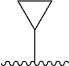 [SMILES: *C1CC1] | N CH CF |

### Compounds of Formula (B6)

Compounds of the general Formula (B6) are described in PCT Publication No. WO 2012/080449, published Jun. 21, 2012, which is hereby incorporated by reference in its entirety. Formula (B6) has the structure:

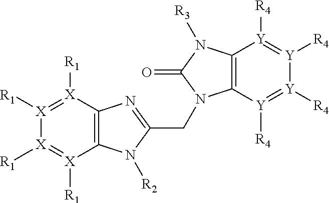

or a prodrug, N-oxide, addition salt, quaternary amine, metal complex, or a stereochemically isomeric form thereof, wherein: each X independently can be C or N; at least one X═N; each Y independently can be C or N; R₁ is present when X═C and R₁ can be selected from H, halogen, C₁-C₆ alkyl, C₃-C₇ cycloalkyl, C₁-C₆ alkoxy, N(R₅)₂, CO(R₆), CH₂NH₂, CH₂OH, CN, C(═NOH)NH₂, C(═NOCH₃)NH₂, C(═NH)NH₂, CF₃, CF₃, and B(OH)₂; B(O—C₁-C₆ alkyl)₂; R₁ is absent when X═N; R₂ can be —(CR₇R₈)ₙ—R₉; R₃ can be selected from H, C₁-C₁₀ alkyl, C₃-C₇ cycloalkyl, C₂-C₁₀ alkenyl, SO₂—R₇; CH₂CF₃ or a 4 to 6 membered saturated ring containing an oxygen atom; R₄ can be present where Y is C and is selected from H, C₁-C₆ alkyl, C₁-C₆ cycloalkyl, C₁-C₆ alkoxy, CO(R₇), COO(R₇), CF₃ and halogen, R₅ can be selected from H, C₁-C₆ alkyl, COOCH₃, and CONHSO₂CH₃; R₆ can be selected from OH, O(C₁-C₆ alkyl), NH₂, NHSO₂N(C₁-C₆ alkyl)₂, NHSO₂NHCH₃, NHSO₂(C₁-C₆ alkyl), NHSO₂(C₃-C₇ cycloalkyl), and N(C₁-C₆-alkyl)₂; R₇ and R₈ can be each independently chosen from H, C₁-C₁₀ alkyl, C₃-C₇ cycloalkyl or R₇ and R₈ can be taken together form a 4 to 6 membered aliphatic ring that optionally contains a heteroatom selected from N, S, O; R₉ can be selected from H, R₁₀, C₁-C₆ alkyl, OH, CN, F, CF₂H, CF₃, CONR₇R₈, COOR₇, CON(R₇)SO₂R₈, CON(R₇)SO₂N(R₇R₈), NR₇R₈, NR₇COOR₈, COR₇, O-Benzyl, NR₇SO₂R₈, SO₂R₇R₈, SO₂R₇, CONR₇R₈, CONR₇R₁₀, N(R₇)CON(R₇R₈), N(R₇)COOC; phtalimido, 2-methyl-benzothiophene(1,1)di oxide, or a 4 to 6 membered saturated ring containing an oxygen atom; n can be an integer from 2 to 6; R₁₀ can be selected from C₁-C₆ alkyl, C₃-C₇ cycloalkyl, phenyl, pyridine or pyrazole, optionally substituted with one or more substituents selected from CF₃, CH₃, CH₃, OCF₃ or halogen.

Examples of Compounds of Formula (B6) include:

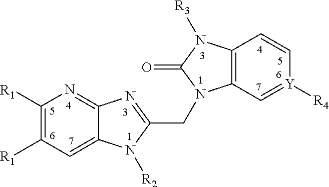

| N^{o} | Name | C₅—R₁ | C₆—R₁ | R₂ | R₃ | Y—R₄ |
|---|---|---|---|---|---|---|
| 1 | 3-((5-chloro-1-isopentyl- 1H-imidazol[4,5-b]pyridin- 2-yl)methyl)-1-cyclopropyl- 1H-imidazo[4,5-c]pyridin- 2(3H)-one | C—Cl | C—H | 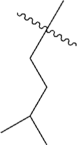 [SMILES: *CCC(C)C] | 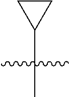 [SMILES: *C1CC1] | N |
| 2 | 3-((5-chloro-1-isopentyl- 1H-imidazo[4,5-b]pyridin- 2-yl)methyl)-1-(oxetan-3- yl)-1H-imidazo[4,5-c] pyridin-2(3H)-one | C—Cl | C—H | 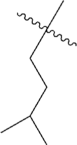 [SMILES: *CCC(C)C] | 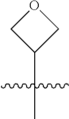 [SMILES: *C1COC1] | N |
| 3 | 4-(5-chloro-2-((1- cyclopropyl-2-oxo-1H- imidazo[4,5-c]pyridin- 3(2H)-yl)methyl)-1H- imidazo[4,5-b]pyridin- 1yl)butyl pivalate | C—Cl | C—H | 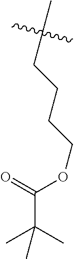 [SMILES: *CCCCOC(=O)C(C)(C)C] | 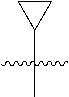 [SMILES: *C1CC1] | N |
| 4 | 1-cyclopropyl-3-((1- isopentyl-1H-imidazo[4,5- b]pyridin-2-yl)methyl)-1H- benzo[d]imidazol-2(3H)-one | C—H | C—H | 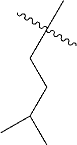 [SMILES: *CCC(C)C] | 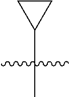 [SMILES: *C1CC1] | C—H |
| 5 | 1-cyclopropyl-3-((1- isopentyl-1H-imidazo[4,5- b]pyridin-2-yl)methyl)-1H- imidazo[4,5-c]pyridin- 2(3H)-one | C—H | C—H | 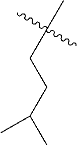 [SMILES: *CCC(C)C] | 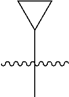 [SMILES: *C1CC1] | N |
| 6 | 3-((5-chloro-1-isopentyl- 1H-imidazo[4,5-b]pyridin- 2-yl)methyl)-1-cyclopropyl- 5-fluoro-1H- benzo[d]imidazol-2(3H)-one | C—Cl | C—H | 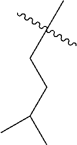 [SMILES: *CCC(C)C] | 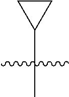 [SMILES: *C1CC1] | C—F |
| 7 | 1-cyclopropyl-3-((1-(3- (methylsulfonyl)propyl)-1H- imidazo[4,5-b]pyridin-2- yl)methyl)-1H-imidazo[4,5- c]pyridin-2(3H)-one | C—H | C—H | 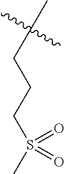 [SMILES: *CCCS(C)(=O)=O] | 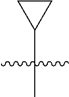 [SMILES: *C1CC1] | N |
| 8 | 1-cyclopropyl-5-fluoro-3- ((1-isopentyl-1H- imidazo[4,5-b]pyridin-2- yl)methyl)-1H- benzo[d]imidazol-2(3H)-one | C—H | C—H | 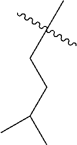 [SMILES: *CCC(C)C] | 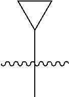 [SMILES: *C1CC1] | C—F |
| 9 | 3-((1-isopentyl-1H- imidazo[4,5-b]pyridin-2- yl)methyl)-1-(oxetan-3-yl)- 1H-imidazo[4,5-c]pyridin- 2(3H)-one | C—H | C—H | 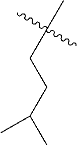 [SMILES: *CCC(C)C] | 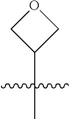 [SMILES: *C1COC1] | N |
| 10 | 1-cyclopropyl-3-((1-(3- (methoxypropyl)-1H- imidazo[4,5-b]pyridin-2- yl)methyl)-1H-imidazo[4,5- c]pyridin-2(3H)-one | C—H | C—H | 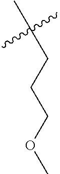 [SMILES: *CCCOC] | 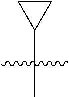 [SMILES: *C1CC1] | N |
| 11 | 1-cyclopropyl-3-((1-(3- (fluoropropyl)-1H- imidazo[4,5-b]pyridin-2- yl)methyl)-1H-imidazo[4,5- c]pyridin-2(3H)-one | C—H | C—H | 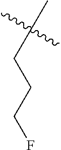 [SMILES: *CCCF] | 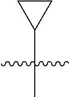 [SMILES: *C1CC1] | N |
| 12 | 3-((1-(3-methoxypropyl)- 1H-imidazo[4,5-b]pyridin- 2-yl)methyl)-1-(oxetan-3- yl)-1H-imidazo[4,5- c]pyridin-2(3H)-one | C—H | C—H | 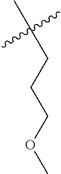 [SMILES: *CCCOC] | 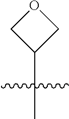 [SMILES: *C1COC1] | N |
| 13 | 3-((5-chloro-1-isopentyl- 1H-imidazo[4,5-b]pyridin- 2-yl)methyl)-3-cyclopropyl- 1H-benzo[d]imidazol- 2(3H)-one | C—Cl | C—H | 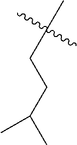 [SMILES: *CCC(C)C] | 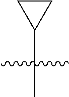 [SMILES: *C1CC1] | C—H |
| 14 | 3-((5-chloro-1-(4- hydroxybutyl)-1H- imidazo[4,5-b]pyridin-2- yl)methyl)-1-cyclopropyl- 1H-imidazo[4,5-c]pyridin- 2(3H)-one | C—Cl | C—H | 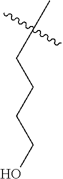 [SMILES: *CCCCO] | 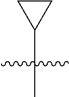 [SMILES: *C1CC1] | N |
| 15 | 1-cyclopropyl-5-fluoro-3- ((1-(3-methoxypropyl)-1H- imidazo[4,5-b]pyridin-2- yl)methyl)-1H- benzo[d]imidazol-2(3H)-one | C—H | C—H | 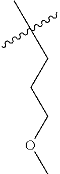 [SMILES: *CCCOC] | 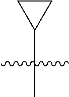 [SMILES: *C1CC1] | C—F |
| 39 | 3-((5-chloro-1-(4,4,4- trifluorobutyl)-1H- imidazo[4,5-b]pyridin-2- yl)methyl)-1-cyclopropyl- 1H-imidazo[4,5-b]pyridin- 2(3H)-one | C—Cl | C—H | 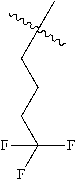 [SMILES: *CCCC(F)(F)F] | 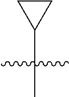 [SMILES: *C1CC1] | N |
| 40 | 3-((5-chloro-1-(4- fluorobutyl)-1H- imidazo[4,5-b]pyridin-2- yl)methyl)-1-cyclopropyl- 1H-imidazo[4,5-b]pyridin- 2(3H)-one | C—Cl | C—H | 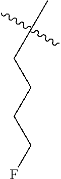 [SMILES: *CCCCF] | 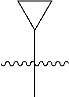 [SMILES: *C1CC1] | N |
| 41 | 1-cyclopropyl-3-((1-(4,4,4- trifluorobutyl)-1H- imidazo[4,5-b]pyridin-2- yl)methyl)-1H-imidazo[4,5- c]pyridin-2(3H)-one | C—H | C—H | 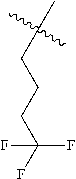 [SMILES: *CCCC(F)(F)F] | 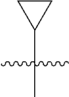 [SMILES: *C1CC1] | N |
| 42 | 1-cyclopropyl-3-((1-(4- fluorobutyl)-1H- imidazo[4,5-b]pyridin-2- yl)methyl)-1H-imidazo[4,5- c]pyridin-2(3H)-one | C—H | C—H | 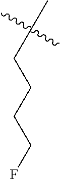 [SMILES: *CCCCF] | 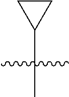 [SMILES: *C1CC1] | N |
| 43 | 1-cyclopropyl-3-((1-(4,4- difluorobutyl)-1H- imidazo[4,5-b]pyridin-2- yl)methyl)-1H-imidazo[4,5- c]pyridin-2(3H)-one | C—H | C—H | 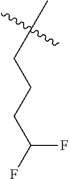 [SMILES: *CCCC(F)F] | 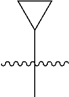 [SMILES: *C1CC1] | N |
| 44 | 1-cyclopropyl-3-((1- isopentyl-5- (trifluoromethyl)-1H- imidazo[4,5-b]pyridin-2- yl)methyl)-1H-imidazo[4,5- c]pyridin-2(3H)-one | C—CF₃ | C—H | 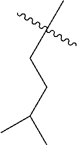 [SMILES: *CCC(C)C] | 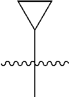 [SMILES: *C1CC1] | N |

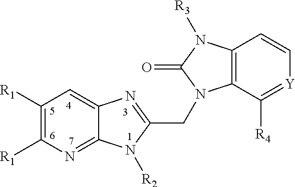

| N^{o} | Name | C₅—R₁ | C₆—R₁ | R₂ | R₃ | R₄ | Y |
|---|---|---|---|---|---|---|---|
| 16 | 3-((6-bromo-3-isopentyl-3H- imidazo[4,5-b]pyridin-2- yl)methyl)-1-cyclopropyl-1H- imidazo[4,5-c]pyridin-2(3H)- one | C—Br | C—H | 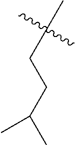 [SMILES: *CCC(C)C] | 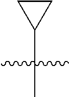 [SMILES: *C1CC1] | H | N |
| 17 | 3-((6-aminomethyl)-3- isopentyl-3H-imidazo[4,5- b]pyridine-2-yl)methyl)-1- cyclopropyl-1H-imidazo[4,5- c]pyridin-2(3H)-one | C—CH₂NH₂ | C—H | 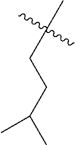 [SMILES: *CCC(C)C] | 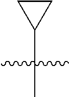 [SMILES: *C1CC1] | H | N |
| 18 | 3-((6-bromo-3-isopentyl-3H- imidazo[4,5-b]pyridin-2- yl)methyl)-1-(oxetan-3-yl)- 1H-imidazo[4,5-c]pyridin- 2(3H)-one | C—Br | C—H | 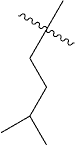 [SMILES: *CCC(C)C] | 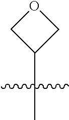 [SMILES: *C1COC1] | H | N |
| 19 | 3-((6-bromo-3-(4- 2(3H)-one 3-((6-bromo-3-(4- hydroxybutyl)-3H- imidazo[4,5-b]pyridin-2- yl)methyl)-1-cyclopropyl-1H- imidazo[4,5-c]pyridin-2(3H)- one | C—Br | C—H | 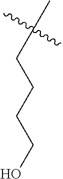 [SMILES: *CCCCO] | 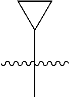 [SMILES: *C1CC1] | H | N |
| 20 | 3-((6-bromo-3-(4- hydroxybutyl)-3H- imidazo[4,5-b]pyridin-2- yl)methyl)-1-(oxetan-3-yl)- 1H-imidazo[4,5-c]pyridin- 2(3H)-one | C—Br | C—H | 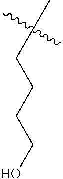 [SMILES: *CCCCO] | 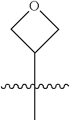 [SMILES: *C1COC1] | H | N |
| 21 | 3-((6-chloro-3-(4- hydroxybutyl)-3H- imidazo[4,5-b]pyridin-2- yl)methyl)-1-cyclopropyl-1H- imidazo[4,5-c]pyridin-2(3H)- one | C—Cl | C—H | 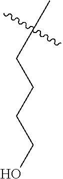 [SMILES: *CCCCO] | 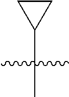 [SMILES: *C1CC1] | H | N |
| 22 | 1-cyclopropyl-3-((6-fluoro-3- (4-hydroxybutyl)-3H- imidazo[4,5-b]pyridin-2- yl)methyl)-1H-imidazo[4,5- c]pyridin-2(3H)-one | C—F | C—H | 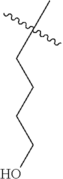 [SMILES: *CCCCO] | 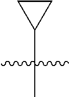 [SMILES: *C1CC1] | H | N |
| 23 | 2-((1-cyclopropyl-2-oxo-1H- imidazo[4,5-c]pyridin-3(2H)- yl)methyl)-3-isopentyl-3H- imidazo[4,5-b]pyridin-6- ylboronic acid | C—B(OH)₂ | C—H | 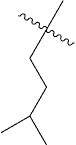 [SMILES: *CCC(C)C] | 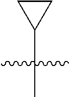 [SMILES: *C1CC1] | H | N |
| 24 | 1-cyclopropyl-3-((3- isopentyl-3H-imidazo[4,5- b]pyridin-2-yl)methyl)-1H- imidazo[4,5-c]pyridin-2(3H)- one | C—H | C—H | 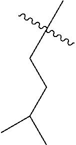 [SMILES: *CCC(C)C] | 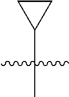 [SMILES: *C1CC1] | H | N |
| 25 | 4-chloro-3-((3-isopentyl-3H- imidazo[4,5-b]pyridin-2- yl)methyl)-1-isopropyl-1H- imidazo[4,5-c]pyridin-2(3H)- one | C—H | C—H | 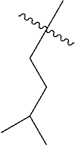 [SMILES: *CCC(C)C] | 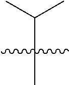 [SMILES: *C(C)C] | Cl | N |
| 26 | methyl 2-((1-cyclopropyl-2- oxo-1H-imidazo[4,5- c]pyridin-3(2H)-yl)methyl)-3- isopentyl-3H-imidazo[4,5- b]pyridine-6-carboxylate | C—CO₂Me | C—H | 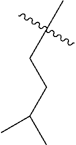 [SMILES: *CCC(C)C] | 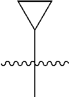 [SMILES: *C1CC1] | H | N |
| 27 | 2-((1-cyclopropyl-2-oxo-1H- imidazo[4,5-c]pyridin-3(2H)- yl)methyl)-3-isopentyl-3H- imidazo[4,5-b]pyridine-6- carbonitrile | C—CN | C—H | 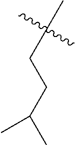 [SMILES: *CCC(C)C] | 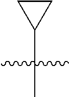 [SMILES: *C1CC1] | H | N |
| 28 | 2-((1-cyclopropyl-2-oxo-1H- imidazo[4,5-c]pyridin-3(2H)- yl)methyl)-3-isopentyl-3H- imidazo[4,5-b]pyridine-6- carboxylic acid | C—CO₂H | C—H | 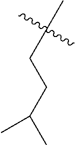 [SMILES: *CCC(C)C] | 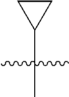 [SMILES: *C1CC1] | H | N |
| 29 | 3-((3-isopentyl-3H- imidazo[4,5-b]pyridin-2- yl)methyl)-1-isopropyl-2- oxo-2,3-dihydro-1H- imidazo[4,5-c]pyridine-4- carbonitrile | C—H | C—H | 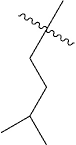 [SMILES: *CCC(C)C] | 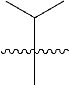 [SMILES: *C(C)C] | CN | N |
| 30 | 1-((6-bromo-3-isopentyl-3H- imidazo[4,5-b]pyridin-2- yl)methyl)-3-cyclopropyl-1H- benzo[d]imidazol-2(3H)-one | C—Br | C—H | 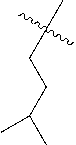 [SMILES: *CCC(C)C] | 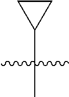 [SMILES: *C1CC1] | H | CH |

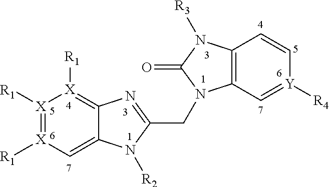

| N^{o} | Name | X₄—R₁ | X₅—R₁ | X₆—R₁ | R₂ | R₃ | Y—R₄ |
|---|---|---|---|---|---|---|---|
| 31 | 1-cyclopropyl-3-((1-isopentyl- 1H-imidazo[4,5-c]pyridin-2- yl)methyl-1H- benzo[d]imidazol-2(3H)-one | C—H | N | C—H | 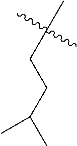 [SMILES: *CCC(C)C] | 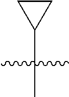 [SMILES: *C1CC1] | C—H |
| 32 | 1-cyclopropyl-5-fluoro-3-((1- isopentyl-1H-imidazo[4,5- c]pyridin-2-yl)methyl-1H- benzo[d]imidazol-2(3H)-one | C—H | N | C—H | 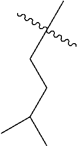 [SMILES: *CCC(C)C] | 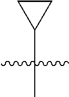 [SMILES: *C1CC1] | C—F |
| 33 | 1-cyclopropyl-3-((1-isopentyl- 1H-imidazo[4,5-c]pyridin-2- yl)methyl-1H-imidazo[4,5- c]pyridin-2(3H)-one | C—H | N | C—H | 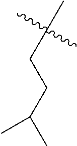 [SMILES: *CCC(C)C] | 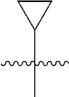 [SMILES: *C1CC1] | N |
| 34 | 1-cyclopropyl-3-((3-isopentyl- 3H-imidazo[4,5-c]pyridin-2- yl)methyl-1H-imidazo[4,5- c]pyridin-2(3H)-one | C—H | C—H | N | 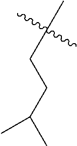 [SMILES: *CCC(C)C] | 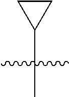 [SMILES: *C1CC1] | N |
| 35 | 1-cyclopropyl-3-((1-(4- hydroxybutyl)-1H-imidazo[4,5- c]pyridin-2-yl)methyl-1H- imidazo[4,5-c]pyridin-2(3H)-one | C—H | N | C—H | 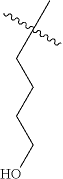 [SMILES: *CCCCO] | 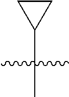 [SMILES: *C1CC1] | N |
| 36 | 3-((1-isopentyl-1H- imidazo[4,5-c]pyridin-2- yl)methyl-1-(oxetan-3-yl)-1H- imidazo[4,5-c]pyridin-2(3H)-one | C—H | N | C—H | 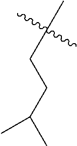 [SMILES: *CCC(C)C] | 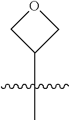 [SMILES: *C1COC1] | N |
| 37 | 1-cyclopropyl-3-((4- (dimethylamino)-1-(4- hydroxybutyl)-1H-imidzo[4,5- c]pyridin-2-yl)methyl-1H- imidazo[4,5-c]pyridin-2(3H)- one | C—NMe₂ | N | C—H | 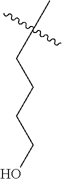 [SMILES: *CCCCO] | 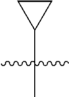 [SMILES: *C1CC1] | N |
| 38 | 3-((1-(4-benzyloxy)butyl)-4- chloro-1H-imidazo[4,5-c] pyridin-2-yl)methyl-1- cyclopropyl-1H-imidazo[4,5- c]pyridin-2(3H)-one | C—Cl | N | C—H | 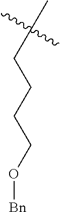 [SMILES: *CCCCOCc1ccccc1] | 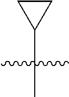 [SMILES: *C1CC1] | N |

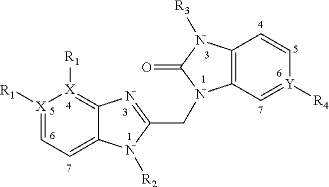

| | X₄—R₁ | X5—R₁ | R₂ | R₃ | |
|---|---|---|---|---|---|
| 99 | N | C—Cl | 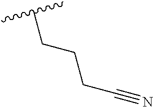 [SMILES: *CCCC#N] | 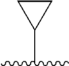 [SMILES: *C1CC1] | |
| 100 | N | C—Cl | 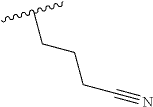 [SMILES: *CCCC#N] | 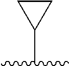 [SMILES: *C1CC1] | |
| 101 | N | C—Cl | 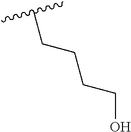 [SMILES: *CCCCO] | 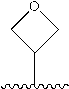 [SMILES: *C1COC1] | |
| | X₄—R₁ | X5—R₁ | R₂ | R₃ | Y—R₄ |
| 102 | N | C—Cl | 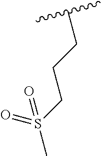 [SMILES: *CCCS(C)(=O)=O] | 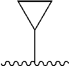 [SMILES: *C1CC1] | N |
| 103 | N | C—Cl | 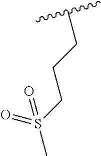 [SMILES: *CCCS(C)(=O)=O] | 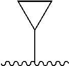 [SMILES: *C1CC1] | C—F |
| 45 | N | C—Cl | 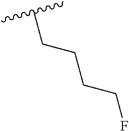 [SMILES: *CCCCF] | 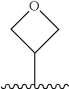 [SMILES: *C1COC1] | N |
| 46 | N | C—Cl | 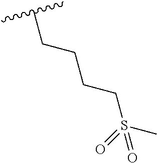 [SMILES: *CCCCS(C)(=O)=O] | 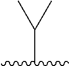 [SMILES: *C(C)C] | C—F |
| 47 | N | C—Cl | 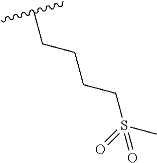 [SMILES: *CCCCS(C)(=O)=O] | 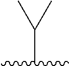 [SMILES: *C(C)C] | N |
| 48 | N | C—Cl | 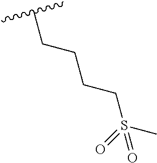 [SMILES: *CCCCS(C)(=O)=O] | 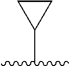 [SMILES: *C1CC1] | C—H |
| 50 | N | C—H | 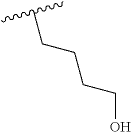 [SMILES: *CCCCO] | H | N |
| 51 | N | C—H | 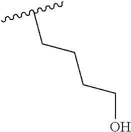 [SMILES: *CCCCO] | 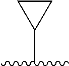 [SMILES: *C1CC1] | N |
| 54 | N | C—Cl | 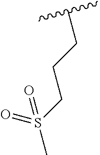 [SMILES: *CCCS(C)(=O)=O] | 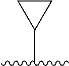 [SMILES: *C1CC1] | C—H |
| 55 | N | C—Cl | 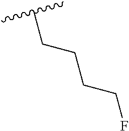 [SMILES: *CCCCF] | 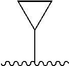 [SMILES: *C1CC1] | C—COOEt |
| 56 | N | C—Cl | 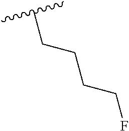 [SMILES: *CCCCF] | 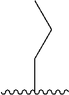 [SMILES: *CCC] | C—COOEt |
| 57 | N | C—Cl | 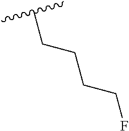 [SMILES: *CCCCF] | 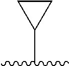 [SMILES: *C1CC1] | C—COOH |
| 58 | N | C—Cl | 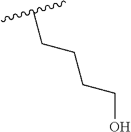 [SMILES: *CCCCO] | 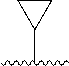 [SMILES: *C1CC1] | C—F |
| 43 | N | C—H | 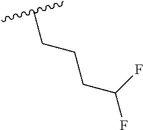 [SMILES: *CCCC(F)F] | 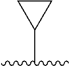 [SMILES: *C1CC1] | N |
| 64 | N | C—Cl | 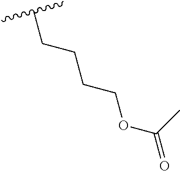 [SMILES: *CCCCOC(C)=O] | 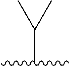 [SMILES: *C(C)C] | N |
| 65 | N | C—Cl | 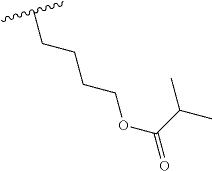 [SMILES: *CCCCOC(=O)C(C)C] | 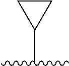 [SMILES: *C1CC1] | N |
| 66 | N | C—Cl | 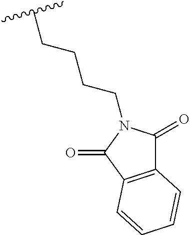 [SMILES: *CCCCN1C(=O)c2ccccc2C1=O] | 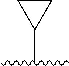 [SMILES: *C1CC1] | N |
| 67 | N | C—Cl | 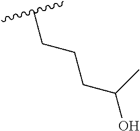 [SMILES: *CCCC(C)O] | 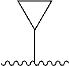 [SMILES: *C1CC1] | N |
| 68 | N | C—Cl | 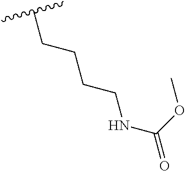 [SMILES: *CCCCNC(=O)OC] | 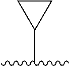 [SMILES: *C1CC1] | N |
| 69 | N | C—F | 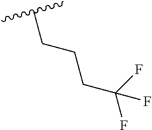 [SMILES: *CCCC(F)(F)F] | 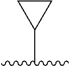 [SMILES: *C1CC1] | N |
| 70 | N | C—H | 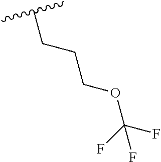 [SMILES: *CCCOC(F)(F)F] | 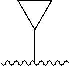 [SMILES: *C1CC1] | N |
| 71 | N | C—Cl | 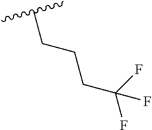 [SMILES: *CCCC(F)(F)F] | 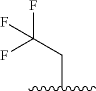 [SMILES: *CC(F)(F)F] | N |
| 72 | N | C—H | 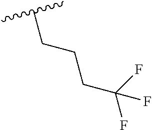 [SMILES: *CCCC(F)(F)F] | 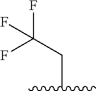 [SMILES: *CC(F)(F)F] | N |
| 73 | N | C—Cl | 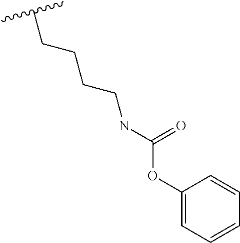 [SMILES: *CCCCNC(=O)Oc1ccccc1] | 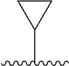 [SMILES: *C1CC1] | N |
| 74 | N | C—Cl | 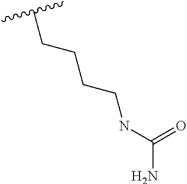 [SMILES: *CCCCNC(N)=O] | 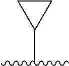 [SMILES: *C1CC1] | N |
| 75 | N | C—Cl | 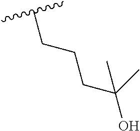 [SMILES: *CCCC(C)(C)O] | 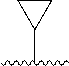 [SMILES: *C1CC1] | N |
| 76 | N | C—Cl | 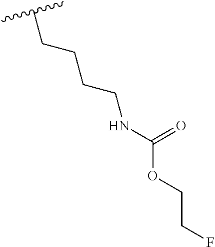 [SMILES: *CCCCNC(=O)OCCF] | 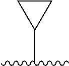 [SMILES: *C1CC1] | N |
| 77 | N | C—Cl | 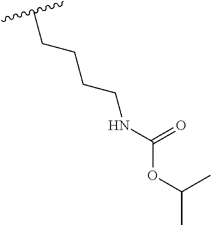 [SMILES: *CCCCNC(=O)OC(C)C] | 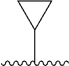 [SMILES: *C1CC1] | N |
| 78 | N | C—Cl | 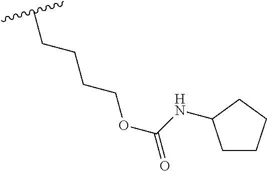 [SMILES: *CCCCOC(=O)NC1CCCC1] | 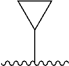 [SMILES: *C1CC1] | N |
| 79 | N | C—Cl | 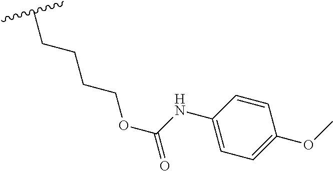 [SMILES: *CCCCOC(=O)Nc1ccc(OC)cc1] | 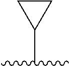 [SMILES: *C1CC1] | N |
| 80 | N | C—Cl | 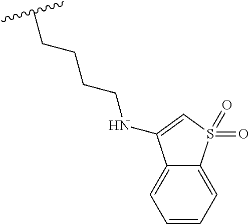 [SMILES: *CCCCNC1=CS(=O)(=O)c2ccccc21] | 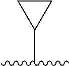 [SMILES: *C1CC1] | N |
| 81 | N | C—H | 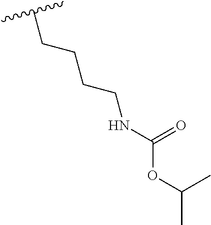 [SMILES: *CCCCNC(=O)OC(C)C] | 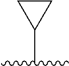 [SMILES: *C1CC1] | N |
| 82 | N | C—Cl | 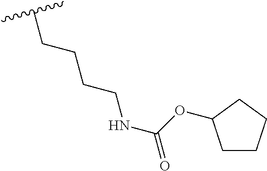 [SMILES: *CCCCNC(=O)OC1CCCC1] | 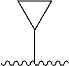 [SMILES: *C1CC1] | N |
| 83 | N | C—Cl | 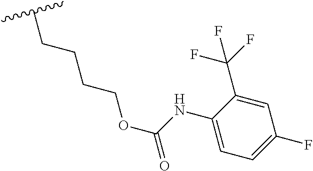 [SMILES: *CCCCOC(=O)Nc1ccc(F)cc1C(F)(F)F] | 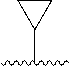 [SMILES: *C1CC1] | N |
| 84 | N | C—Cl | 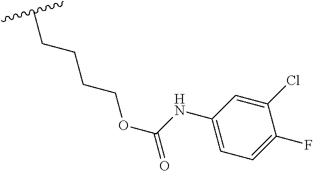 [SMILES: *CCCCOC(=O)Nc1ccc(F)c(Cl)c1] | 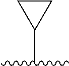 [SMILES: *C1CC1] | N |
| 85 | N | C—H | 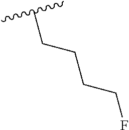 [SMILES: *CCCCF] | 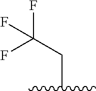 [SMILES: *CC(F)(F)F] | N |
| 86 | N | C—Cl | 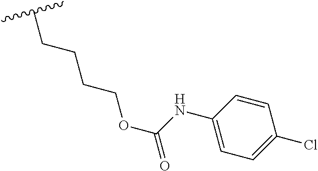 [SMILES: *CCCCOC(=O)Nc1ccc(Cl)cc1] | 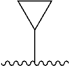 [SMILES: *C1CC1] | N |
| 87 | N | C—Cl | 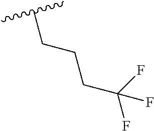 [SMILES: *CCCC(F)(F)F] | 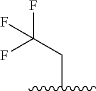 [SMILES: *CC(F)(F)F] | C—F |
| 88 | N | C—Cl | 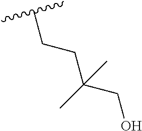 [SMILES: *CCC(C)(C)CO] | 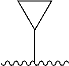 [SMILES: *C1CC1] | N |
| 89 | N | C—H | 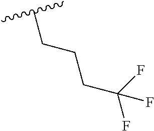 [SMILES: *CCCC(F)(F)F] | 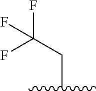 [SMILES: *CC(F)(F)F] | C—F |
| 90 | N | C—Cl | 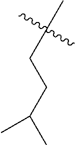 [SMILES: *CCC(C)C] | 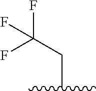 [SMILES: *CC(F)(F)F] | N |
| 91 | N | C—H | 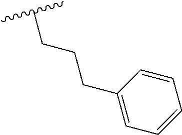 [SMILES: *CCCc1ccccc1] | 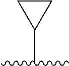 [SMILES: *C1CC1] | N |
| 92 | N | C—Cl | 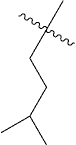 [SMILES: *CCC(C)C] | 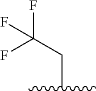 [SMILES: *CC(F)(F)F] | C—F |
| 93 | N | C—H | 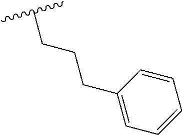 [SMILES: *CCCc1ccccc1] | 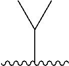 [SMILES: *C(C)C] | C—F |
| 94 | N | C—H | 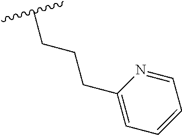 [SMILES: *CCCc1ccccn1] | 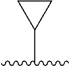 [SMILES: *C1CC1] | N |
| 95 | N | C—H | 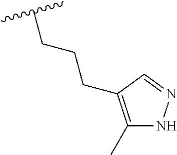 [SMILES: *CCCc1cn[nH]c1C] | 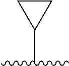 [SMILES: *C1CC1] | C—F |
| 96 | N | C—H | 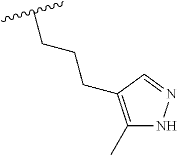 [SMILES: *CCCc1cn[nH]c1C] | 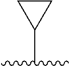 [SMILES: *C1CC1] | N |
| 97 | N | CMe₂NH₂ | 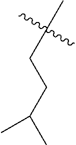 [SMILES: *CCC(C)C] | 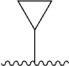 [SMILES: *C1CC1] | N |
| 98 | N | C—H | 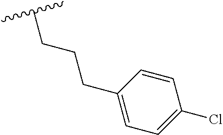 [SMILES: *CCCc1ccc(Cl)cc1] | 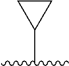 [SMILES: *C1CC1] | N |

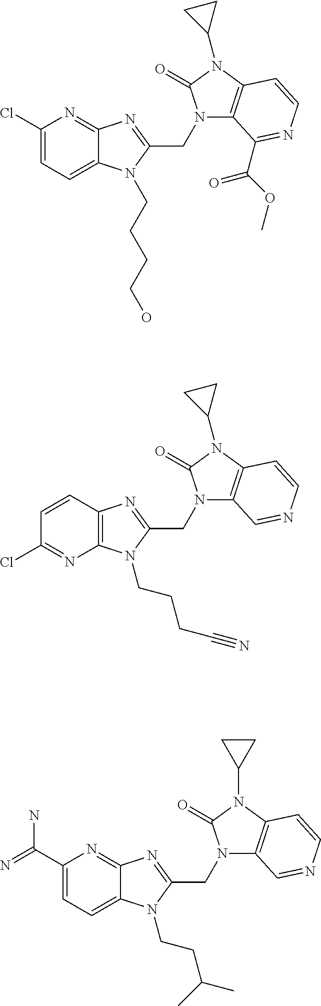 [SMILES: COC(=O)c1nccc2c1n(Cc1nc3nc(Cl)ccc3n1CCCCO)c(=O)n2C1CC1 ; N#CCCCn1c(Cn2c(=O)n(C3CC3)c3ccncc32)nc2ccc(Cl)nc21 ; CC(C)CCn1c(Cn2c(=O)n(C3CC3)c3ccncc32)nc2nc(C(=N)N)ccc21]

### Compounds of Formula (B7)

Compounds of the general Formula (B7) are described in PCT Publication No. WO 2012/080450, published Jun. 21, 2012, which is hereby incorporated by reference in its entirety. Formula (B7) has the structure:

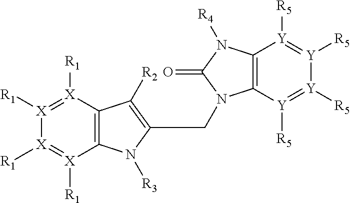

or a prodrug, N-oxide, addition salt, quaternary amine, metal complex, or a stereochemically isomeric form thereof, wherein: each X independently can be C or N with at least one X being N; R₁ is present where X═C and R₁ can be selected from H, OH, halogen, C₁-C₆ alkyl, C₃-C₇ cycloalkyl, C₁-C₆ alkoxy, NH₂, CO(R₇), CH₂NH₂, CH₂OH, CN, C(═NOH)NH₂, C(═NOCH₃)NH₂, C(═NH)NH₂, CF₃, CF₃, and B(OH)₂; B(O—C₁-C₆ alkyl)₂; R₂ can be selected from H, halogen, C₁-C₆ alkyl, C₃-C₇ cycloalkyl, C₁-C₆ alkoxy, and CO(R₇); R₃ can be —(CR₈R₉)ₙ—R₁₀; R₄ can be selected from H, C₁-C₁₀ alkyl, CH₂CF₃C₃-C₇ cycloalkyl, C₂-C₁₀ alkenyl, SO₂—R₈, or a 4 to 6 membered saturated ring containing an oxygen atom; R₅ is present where Y is C, and can be selected from H, C₁-C₆ alkyl, C₃-C₇ cycloalkyl, C₁-C₆ alkoxy, CO(R₇), CF₃ and halogen; R₅ is absent where X is N; R₆ can be selected from H, C₁-C₆ alkyl, COOCH₃, and CONHSO₂CH₃; R₇ can be selected from OH, O(C₁-C₆ alkyl), NH₂, NHSO₂N(C₁-C₆ alkyl)₂, NHSO₂NHCH₃, NHSO₂(C₁-C₆ alkyl), NHSO₂(C₃-C₇ cycloalkyl), and N(C₁-C₆-alkyl)₂; n can be an integer from 2 to 6; R₈ and R₉ can be each independently chosen from H, C₁-C₁₀ alkyl, C₃-C₇ cycloalkyl or R₈ and R₉ can be taken together form a 4 to 6 membered aliphatic ring that optionally contains a heteroatom selected from N, S, O; R₁₀ can be selected from H, C₁-C₆ alkyl, OH, CN, F, CF₂H, CF₃, CONR₈R₉, COOR₈, CONR₈SO₂R₉, CON(R₈)SO₂N(R₈R₉), NR₈R₉, NR₈COOR₉, COR₈, NR₈SO₂R₉, SO₂NR₈R₉, SO₂R₈ or a 4 to 6 membered saturated ring containing an oxygen atom.

Examples of Compounds of Formula (B7) include:

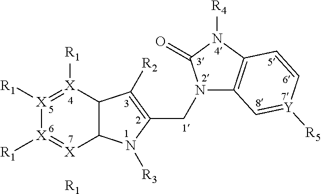

| | Compound name | X₄—R₁ | X₅—R₁ | X₆—R₁ | X₇—R₁ | R₂ | R₃ | R₄ | Y_{7′}—R₅ |
|---|---|---|---|---|---|---|---|---|---|
| P1 | 1-Cyclopropyl-3- {[1-(4-hydroxybutyl)- 1H-pyrrolo[3,2-c] pyridin-2-yl]methyl}-1, 3-dihydro-2H-imidazo [4,5-c]pyridin-2-one | C—H | N | C—H | C—H | H | 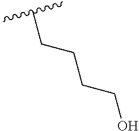 [SMILES: *CCCCO] | 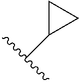 [SMILES: *C1CC1] | N |
| P2 | 3-{[5-Chloro-1-(4- hydroxybutyl)-1H- pyrrolo[3,2-b]pyridin-2- yl]methyl}-1-cyclopropyl- 1,3-dihydro-2H-imidazo [4,5-c]pyridin-2-one | N | C—Cl | C—H | C—H | H | 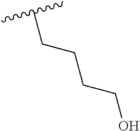 [SMILES: *CCCCO] | 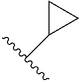 [SMILES: *C1CC1] | N |
| P3 | 1-Cyclopropyl-3- {[1-(3-methylbutyl)-1H- pyrrolo[3,2-c]pyridin- 2-yl]methyl}-1,3- dihydro-2H-imidazo [4,5-c]pyridin-2- one | N | C—H | C—H | C—H | H | 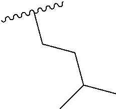 [SMILES: *CCC(C)C] | 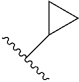 [SMILES: *C1CC1] | N |
| P4 | 1-Cyclopropyl-3- {[1-(4-hydroxybutyl)-1H- pyrrolo[2,3-c]pyridin-2- yl]methyl}-1,3-dihydro- 2H-imidazo[4,5-c] pyridin-2-one | C—H | C—H | N | C—H | H | 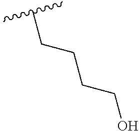 [SMILES: *CCCCO] | 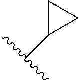 [SMILES: *C1CC1] | N |
| P5 | 1-Cyclopropyl-3-{[1-(4- hydroxybutyl)-1H-pyrrolo [3,2-b]pyridin-2-yl] methyl}-1,3-dihydro- 2H-imidazo[4,5-c] pyridin-2-one | N | C—H | C—H | C—H | H | 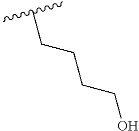 [SMILES: *CCCCO] | 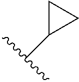 [SMILES: *C1CC1] | N |
| P6 | 1-Cyclopropyl-3-{[1-(4- hydroxybutyl)-5- methoxy-1H-pyrrolo [3,2-b]pyridin-2-yl] methyl}-1,3-dihydro- 2H-imidazo[4,5-c] pyridin-2-one | N | C—OMe | C—H | C—H | H | 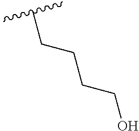 [SMILES: *CCCCO] | 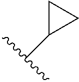 [SMILES: *C1CC1] | N |
| P8 | 1-Cyclopropyl-3-{[5- hydroxy-1-(4- hydroxybutyl)-1H- pyrrolo[3,2-b]pyridin-2- yl]methyl}-1,3- dihydro-2H-imidazo [4,5-c]pyridin-2-one | N | C—OH | C—H | C—H | H | 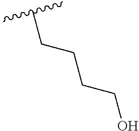 [SMILES: *CCCCO] | 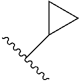 [SMILES: *C1CC1] | N |
| P9 | 4-{2-[(1-Cyclopropyl- 2-oxo-1,2-dihydro- 3H-imidazo[4,5-c] pyridin-3-yl)methyl]- 5-hydroxy-1H- pyrrolo[3,2-b] pyridin-1-yl}butyl dimethylpropanoate | N | C—OH | C—H | C—H | H | 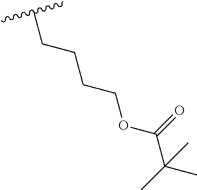 [SMILES: *CCCCOC(=O)C(C)(C)C] | 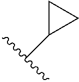 [SMILES: *C1CC1] | N |
| P10 | 1-Cyclopropyl-3-{[1- (3-methylbutyl)- 1H-pyrrolo[2,3-b] pyridin-2-yl]methyl}- 1,3-dihydro-2H- imidazo[4,5- c]pyridin-2-one | C—H | C—H | C—H | N | H | 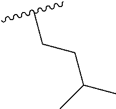 [SMILES: *CCC(C)C] | 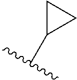 [SMILES: *C1CC1] | N |

| | X₄—R₁ | X₅—R₁ | X₆—R₁ | R₂ | R₃ | R₄ | Y_{7′}—R₅ |
|---|---|---|---|---|---|---|---|
| P11 | H | C—Cl | N | H | 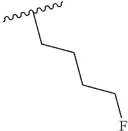 [SMILES: *CCCCF] | 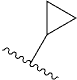 [SMILES: *C1CC1] | N |
| P12 | CH | C—Cl | N | H | 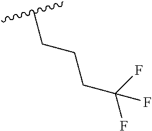 [SMILES: *CCCC(F)(F)F] | 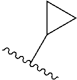 [SMILES: *C1CC1] | N |
| P13 | CH | C—Cl | N | H | 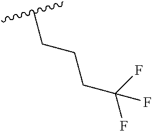 [SMILES: *CCCC(F)(F)F] | 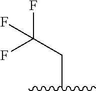 [SMILES: *CC(F)(F)F] | N |
| P14 | CH | C—Cl | N | H | 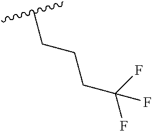 [SMILES: *CCCC(F)(F)F] | 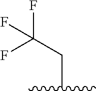 [SMILES: *CC(F)(F)F] | C—F |
| P15 | CH | C—Cl | N | H | 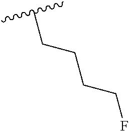 [SMILES: *CCCCF] | 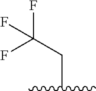 [SMILES: *CC(F)(F)F] | N |
| P16 | CH | C—Cl | N | H | 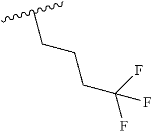 [SMILES: *CCCC(F)(F)F] | 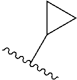 [SMILES: *C1CC1] | C—F |
| P17 | CH | N | CH | Br | 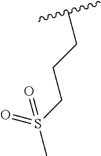 [SMILES: *CCCS(C)(=O)=O] | 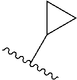 [SMILES: *C1CC1] | N |
| P18 | CH | N | CH | H | 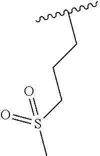 [SMILES: *CCCS(C)(=O)=O] | 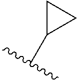 [SMILES: *C1CC1] | N |
| P19 | CH | N | CH | H | 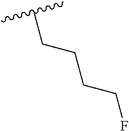 [SMILES: *CCCCF] | 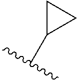 [SMILES: *C1CC1] | N |
| P20 | CH | N | CH | H | 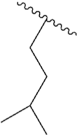 [SMILES: *CCC(C)C] | 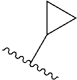 [SMILES: *C1CC1] | N |
| P21 | CH | N | CH | H | 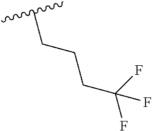 [SMILES: *CCCC(F)(F)F] | 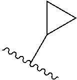 [SMILES: *C1CC1] | N |
| P22 | CH | N | CH | Cl | 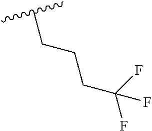 [SMILES: *CCCC(F)(F)F] | 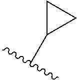 [SMILES: *C1CC1] | N |
| P23 | CH | N | CH | F | 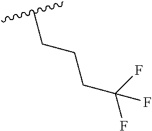 [SMILES: *CCCC(F)(F)F] | 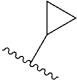 [SMILES: *C1CC1] | N |
| P25 | CH | N | CH | Me | 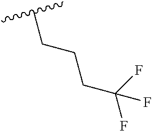 [SMILES: *CCCC(F)(F)F] | 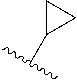 [SMILES: *C1CC1] | N |
| P26 | CH | N | CH | I | 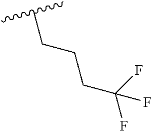 [SMILES: *CCCC(F)(F)F] | 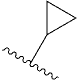 [SMILES: *C1CC1] | N |
| P29 | CH | N | CH | Br | 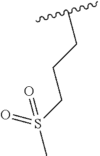 [SMILES: *CCCS(C)(=O)=O] | 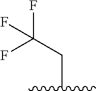 [SMILES: *CC(F)(F)F] | C—F |
| P30 | CH | N | CH | H | 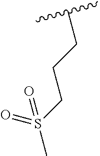 [SMILES: *CCCS(C)(=O)=O] | 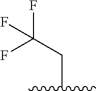 [SMILES: *CC(F)(F)F] | C—F |
| P32 | N | Cl | CH | H | 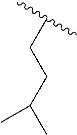 [SMILES: *CCC(C)C] | 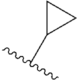 [SMILES: *C1CC1] | N |
| P33 | N | Cl | CH | H | 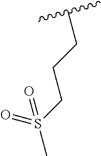 [SMILES: *CCCS(C)(=O)=O] | 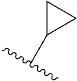 [SMILES: *C1CC1] | N |
| P34 | N | Cl | CH | H | 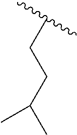 [SMILES: *CCC(C)C] | 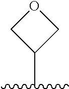 [SMILES: *C1COC1] | N |
| P35 | N | Cl | CH | H | 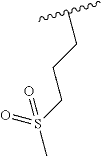 [SMILES: *CCCS(C)(=O)=O] | 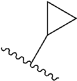 [SMILES: *C1CC1] | C—H |
| P36 | N | Cl | CH | H | 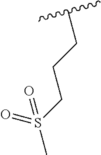 [SMILES: *CCCS(C)(=O)=O] | 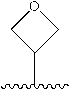 [SMILES: *C1COC1] | N |
| P37 | N | Cl | CH | H | 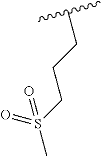 [SMILES: *CCCS(C)(=O)=O] | 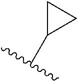 [SMILES: *C1CC1] | C—F |
| P38 | N | Cl | CH | H | 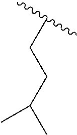 [SMILES: *CCC(C)C] | 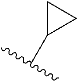 [SMILES: *C1CC1] | C—F |
| P39 | N | Cl | CH | H | 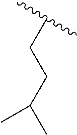 [SMILES: *CCC(C)C] | 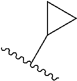 [SMILES: *C1CC1] | C—H |
| P40 | N | Cl | CH | H | 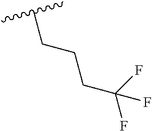 [SMILES: *CCCC(F)(F)F] | 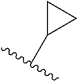 [SMILES: *C1CC1] | N |
| P42 | N | Cl | CH | H | 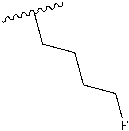 [SMILES: *CCCCF] | 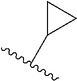 [SMILES: *C1CC1] | N |
| P43 | N | Cl | CH | H | 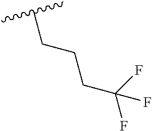 [SMILES: *CCCC(F)(F)F] | 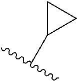 [SMILES: *C1CC1] | C—H |
| P45 | N | CH | CH | H | 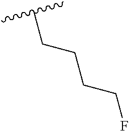 [SMILES: *CCCCF] | 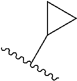 [SMILES: *C1CC1] | N |
| P46 | N | CF₃ | CH | H | 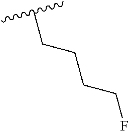 [SMILES: *CCCCF] | 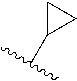 [SMILES: *C1CC1] | N |
| P47 | N | Cl | CH | H | 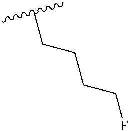 [SMILES: *CCCCF] | 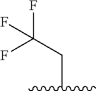 [SMILES: *CC(F)(F)F] | C—F |
| P48 | N | Cl | CH | H | 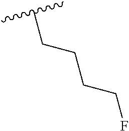 [SMILES: *CCCCF] | 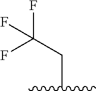 [SMILES: *CC(F)(F)F] | N |
| P49 | N | Cl | CH | H | 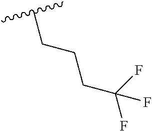 [SMILES: *CCCC(F)(F)F] | 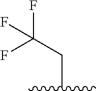 [SMILES: *CC(F)(F)F] | N |
| P50 | N | Cl | CH | H | 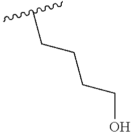 [SMILES: *CCCCO] | 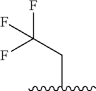 [SMILES: *CC(F)(F)F] | N |
| P51 | N | Cl | CH | H | 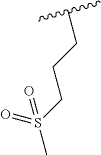 [SMILES: *CCCS(C)(=O)=O] | 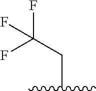 [SMILES: *CC(F)(F)F] | C—H |
| P52 | N | Cl | CH | H | 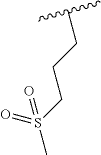 [SMILES: *CCCS(C)(=O)=O] | 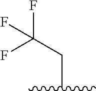 [SMILES: *CC(F)(F)F] | N |
| P53 | N | Cl | CH | H | 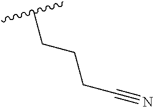 [SMILES: *CCCC#N] | 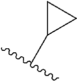 [SMILES: *C1CC1] | N |
| P54 | N | Cl | CH | H | 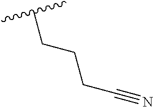 [SMILES: *CCCC#N] | 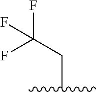 [SMILES: *CC(F)(F)F] | N |

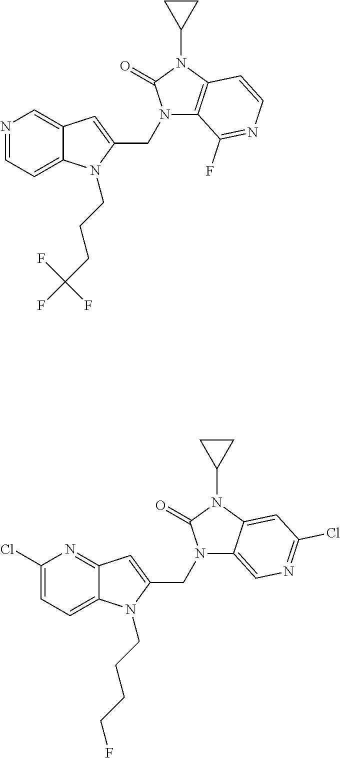 [SMILES: O=c1n(Cc2cc3cnccc3n2CCCC(F)(F)F)c2c(F)nccc2n1C1CC1 ; O=c1n(Cc2cc3nc(Cl)ccc3n2CCCCF)c2cnc(Cl)cc2n1C1CC1]

### Compounds of Formula (B8)

Compounds of the general Formula (B8) are described in PCT Publication No. WO 2012/080451, published Jun. 21, 2012, which is hereby incorporated by reference in its entirety. Formula (B8) has the structure:

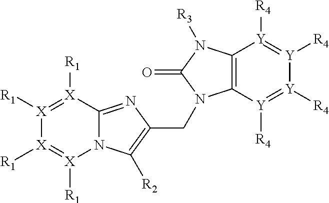

or a prodrug, N-oxide, addition salt, quaternary amine, metal complex, or a stereochemically isomeric form thereof, wherein: each X independently can be C or N; each Y independently can be C or N; R₁ is present when X═C and R₁ can be selected from H, halogen, C₁-C₆ alkyl, C₃-C₇ cycloalkyl, C₁-C₆ alkoxy, N(R₅)₂, CO(R₆), CH₂NH₂, CH₂OH, CN, C(═NOH)NH₂, C(═NOCH₃)NH₂, C(═NH)NH₂, CF₃, CF₃, and B(OH)₂; B(O—C₁-C₆ alkyl)₂; R₁ is absent when X═N; R₂ can be selected from H, halogen, —(CR₇R₈)ₙ—R₉, C≡C—CH₂—R₉ and C≡C—R₉, C═C—R₉; R₃ can be selected from H, C₁-C₁₀ alkyl, C₃-C₇ cycloalkyl, C₂-C₁₀ alkenyl, SO₂—R₇, or a 4 to 6 membered saturated ring containing an oxygen atom; R₄ is present where Y is C and can be selected from H, C₁-C₆ alkyl, C₁-C₆ cycloalkyl, C₁-C₆ alkoxy, CO(R₇), CF₃ and halogen, R₅ can be selected from H, C₁-C₆ alkyl, COOCH₃, and CONHSO₂CH₃; R₆ can be selected from OH, O(C₁-C₆ alkyl), NH₂, NHSO₂N(C₁-C₆ alkyl)₂, NHSO₂NHCH₃, NHSO₂(C₁-C₆ alkyl), NHSO₂(C₃-C₇ cycloalkyl), and N(C₁-C₆-alkyl)₂; R₇ and R₈ can be each independently chosen from H, C₁-C₁₀ alkyl, C₃-C₇ cycloalkyl or R₇ and R₈ can be taken together form a 4 to 6 membered aliphatic ring that optionally contains at least one heteroatom selected from N, S, O; R₉ can be selected from H, C₁-C₆ alkyl, C₁-C₆ alkoxy, C₃-C₇ cycloalkyl OH, CN, F, CF₂H, CF₃, CONR₇R₈, COOR₈, CON(R₇)SO₂R₈, CON(R₇)SO₂N(R₇R₈), NR₇R₈, NR₇COOR₈, COR₇, NR₇SO₂R₈, SO₂NR₇R₈, SO₂R₇ or a 4 to 6 membered saturated ring containing an oxygen atom; n can be an integer from 2 to 6.

Examples of Compounds of Formula (B8) include:

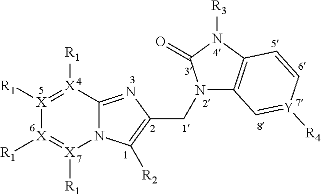

| | X₄—R₁ | X₅—R₁ | X₆—R₁ | X₇—R₁ | R₂ | R₃ | Y_{7′}—R₄ |
|---|---|---|---|---|---|---|---|
| P1 | C—H | C—Cl | C—H | C—H | 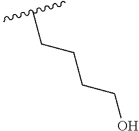 [SMILES: *CCCCO] | 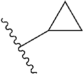 [SMILES: *C1CC1] | N |
| P2 | C—H | C—Cl | C—H | C—H | 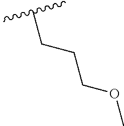 [SMILES: *CCCOC] | 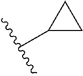 [SMILES: *C1CC1] | N |
| P3 | C—H | C—H | C—H | C—H | 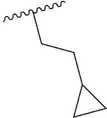 [SMILES: *CCC1CC1] | 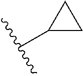 [SMILES: *C1CC1] | N |
| P4 | C—H | C—H | C—H | C—H | 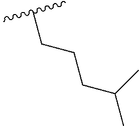 [SMILES: *CCCC(C)C] | 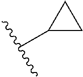 [SMILES: *C1CC1] | N |
| P5 | C—H | C—H | C—H | C—H | 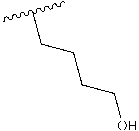 [SMILES: *CCCCO] | 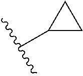 [SMILES: *C1CC1] | N |
| P6 | C—H | C—Cl | C—H | C—H | 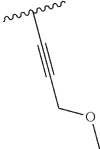 [SMILES: *C#CCOC] | 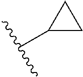 [SMILES: *C1CC1] | N |
| P7 | C—H | C—H | C—H | C—H | 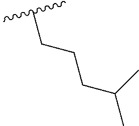 [SMILES: *CCCC(C)C] | 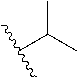 [SMILES: *C(C)C] | C—H |
| P8 | C—H | C—H | C—H | C—H | 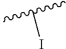 [SMILES: *I] | 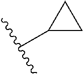 [SMILES: *C1CC1] | N |
| P9 | C—H | C—H | C—H | C—H | 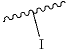 [SMILES: *I] | 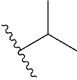 [SMILES: *C(C)C] | C—H |
| P10 | C—H | C—C₃H₈OH | C—H | C—H | 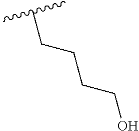 [SMILES: *CCCCO] | 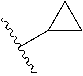 [SMILES: *C1CC1] | N |
| P11 | C—H | C—H | C—H | C—H | 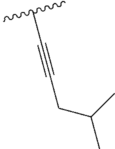 [SMILES: *C#CCC(C)C] | 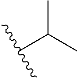 [SMILES: *C(C)C] | C—H |
| P12 | C—H | C—H | C—H | C—H | 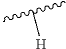 [SMILES: *[H]] | 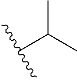 [SMILES: *C(C)C] | C—H |
| P13 | C—H | C—H | C—H | C—H | 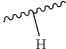 [SMILES: *[H]] | 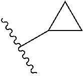 [SMILES: *C1CC1] | N |
| P14 | C—H | N | C—H | C—H | 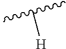 [SMILES: *[H]] | 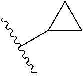 [SMILES: *C1CC1] | N |

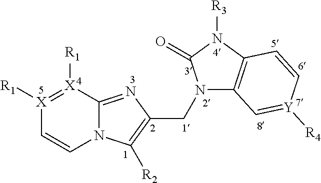

| | X₄—R₁ | X₅—R₁ | R₂ | R₃ | Y_{7′}—R₄ |
|---|---|---|---|---|---|
| P15 | CH | N | 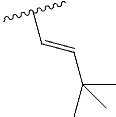 [SMILES: */C=C/C(C)(C)C] | 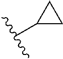 [SMILES: *C1CC1] | N |
| P16 | CH | N | 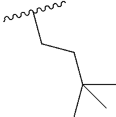 [SMILES: *CCC(C)(C)C] | 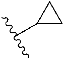 [SMILES: *C1CC1] | N |
| P17 | CH | N | 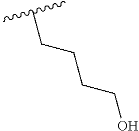 [SMILES: *CCCCO] | 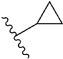 [SMILES: *C1CC1] | N |
| P18 | CH | N | 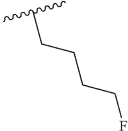 [SMILES: *CCCCF] | 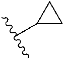 [SMILES: *C1CC1] | N |
| P19 | CH | C—Cl | 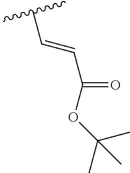 [SMILES: */C=C/C(=O)OC(C)(C)C] | 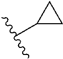 [SMILES: *C1CC1] | N |
| P20 | CH | C—Cl | 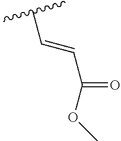 [SMILES: */C=C/C(=O)OC] | 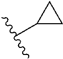 [SMILES: *C1CC1] | N |
| P21 | CH | C—Cl | 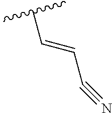 [SMILES: */C=C/C#N] | 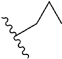 [SMILES: *CCC] | N |
| P22 | CH | C—Cl | 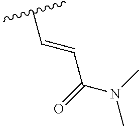 [SMILES: */C=C/C(=O)N(C)C] | 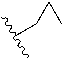 [SMILES: *CCC] | N |
| P23 | CH | C—Cl | 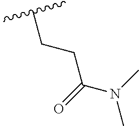 [SMILES: *CCC(=O)N(C)C] | 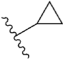 [SMILES: *C1CC1] | N |
| P24 | CH | C—Cl | 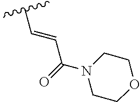 [SMILES: */C=C/C(=O)N1CCOCC1] | 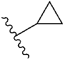 [SMILES: *C1CC1] | N |
| P25 | CH | CH | 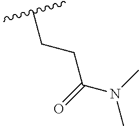 [SMILES: *CCC(=O)N(C)C] | 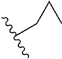 [SMILES: *CCC] | N |
| P26 | CH | C—Cl | 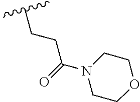 [SMILES: *CCC(=O)N1CCOCC1] | 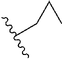 [SMILES: *CCC] | N |
| P27 | CH | CH | 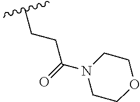 [SMILES: *CCC(=O)N1CCOCC1] | 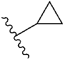 [SMILES: *C1CC1] | N |

### Compounds of Formula (B9)

Compounds of the general Formula (B9) are described in PCT Publication No. WO 2012/080446, published Jun. 21, 2012, which is hereby incorporated by reference in its entirety. Formula (B9) has the structure:

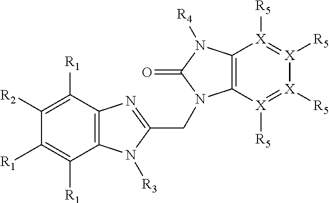

or a prodrug, N-oxide, addition salt, quaternary amine, metal complex, or a stereochemically isomeric form thereof, wherein: each X independently can be C or N; R₁ can be H; R₂ can be selected from Br and Cl; R₃ can be —(CR₆R₇)ₙ—R₈, R₄ can be selected from H, C₃-C₇ cycloalkyl, C₂-C₁₀ alkenyl, —(CR₆R₇)ₙ—R₈, —CH₂-p-Fluorophenyl, CH₂CF₃ and —SO₂CH₃; R₅ is present where X is C, whereby each R₅ can be selected, each independently, from the group consisting of H, C₁-C₆ alkyl, C₁-C₆ alkoxy, halogen, and CN; R₅ is absent where X is N; R₆ and R₇ can be each independently chosen from H and C₁-C₁₀ alkyl, C₃-C₇ cycloalkyl; or R₆ and R₇ can be taken together form a 5 to 6 membered aliphatic or aromatic ring that optionally contains one or more heteroatoms selected from the group N, S, O; R₈ can be selected from H, OH, CF₃, CHF₂, F, CI, SO₂CH₃, SO₂C₃-C₇ cycloalkyl, NR₆SO₂R₆, SO₂R₆R₇, R₆SO₂C₃-C₇ cycloalkyl, CN, NR₆R₇, COOH, COOR₆, CONR₆R₇, OCOC₁-C₆ alkyl, CONR₆SOR₇, CONH—R₆—SO₂R₇, CONH—R₆—SO₂NR₆R₇CONR₆SO₂NR₆R₇, phtalimido or a 5 to 6 membered aliphatic or aromatic ring that optionally contains one or more heteroatoms selected from the group N, S, O; n can be an integer having a value from 1 to 6.

Examples of Compounds of Formula (B9) include:

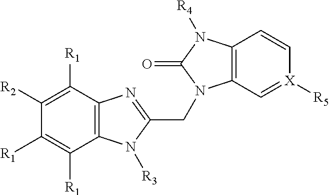

| | R₁ | R₂ | R₃ | R₄ | X—R₅ |
|---|---|---|---|---|---|
| P1 | H | Br | 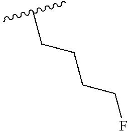 [SMILES: *CCCCF] | 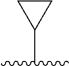 [SMILES: *C1CC1] | N |
| P2 | H | Br | 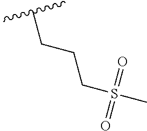 [SMILES: *CCCS(C)(=O)=O] | 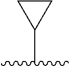 [SMILES: *C1CC1] | N |
| P3 | H | Br | 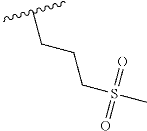 [SMILES: *CCCS(C)(=O)=O] | 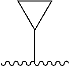 [SMILES: *C1CC1] | C—F |
| P4 | H | Br | 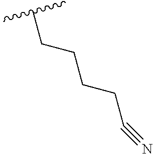 [SMILES: *CCCCC#N] | 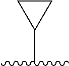 [SMILES: *C1CC1] | N |
| P5 | H | Br | 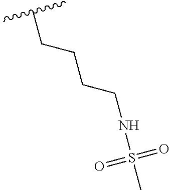 [SMILES: *CCCCNS(C)(=O)=O] | 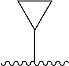 [SMILES: *C1CC1] | N |
| P6 | H | Cl | 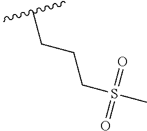 [SMILES: *CCCS(C)(=O)=O] | 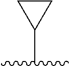 [SMILES: *C1CC1] | N |
| P7 | H | Br | 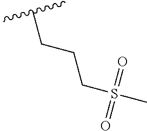 [SMILES: *CCCS(C)(=O)=O] | 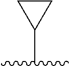 [SMILES: *C1CC1] | C—H |
| P8 | H | Br | 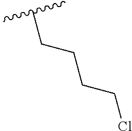 [SMILES: *CCCCCl] | 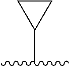 [SMILES: *C1CC1] | N |
| P9 | H | Br | 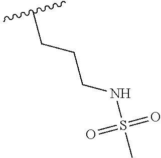 [SMILES: *CCCNS(C)(=O)=O] | 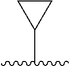 [SMILES: *C1CC1] | N |
| P10 | H | Cl | 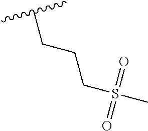 [SMILES: *CCCS(C)(=O)=O] | 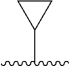 [SMILES: *C1CC1] | C—F |
| P11 | H | Cl | 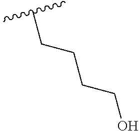 [SMILES: *CCCCO] | 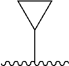 [SMILES: *C1CC1] | C—F |
| P12 | H | Cl | 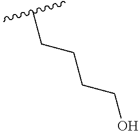 [SMILES: *CCCCO] | 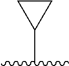 [SMILES: *C1CC1] | C—H |
| P13 | H | Cl | 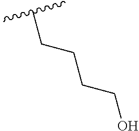 [SMILES: *CCCCO] | 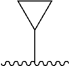 [SMILES: *C1CC1] | N |
| P14 | H | Br | 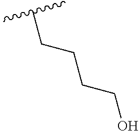 [SMILES: *CCCCO] | 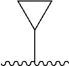 [SMILES: *C1CC1] | N |
| P15 | H | Cl | 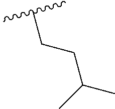 [SMILES: *CCC(C)C] | 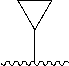 [SMILES: *C1CC1] | N |
| P16 | H | Br | 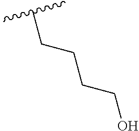 [SMILES: *CCCCO] | 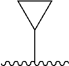 [SMILES: *C1CC1] | C—F |
| P17 | H | Br | 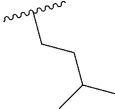 [SMILES: *CCC(C)C] | 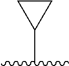 [SMILES: *C1CC1] | N |
| P18 | H | Br | 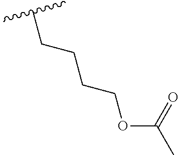 [SMILES: *CCCCOC(C)=O] | 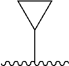 [SMILES: *C1CC1] | N |
| P19 | H | Cl | 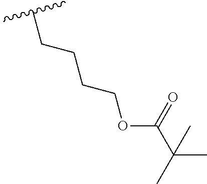 [SMILES: *CCCCOC(=O)C(C)(C)C] | 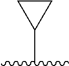 [SMILES: *C1CC1] | C—H |
| P20 | H | Cl | 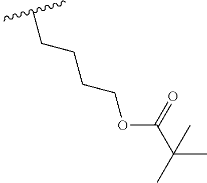 [SMILES: *CCCCOC(=O)C(C)(C)C] | 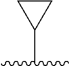 [SMILES: *C1CC1] | C—F |
| P21 | H | Br | 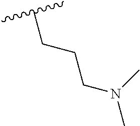 [SMILES: *CCCN(C)C] | 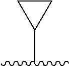 [SMILES: *C1CC1] | N |
| P22 | H | Br | 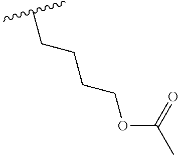 [SMILES: *CCCCOC(C)=O] | 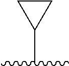 [SMILES: *C1CC1] | C—F |
| P23 | H | Br | 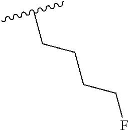 [SMILES: *CCCCF] | 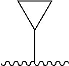 [SMILES: *C1CC1] | C—F |
| P24 | H | Cl | 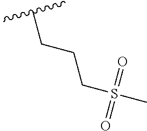 [SMILES: *CCCS(C)(=O)=O] | 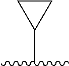 [SMILES: *C1CC1] | C—H |
| P25 | H | Cl | 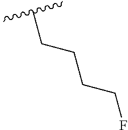 [SMILES: *CCCCF] | 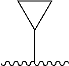 [SMILES: *C1CC1] | C—H |
| P26 | H | Cl | 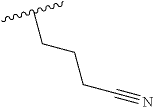 [SMILES: *CCCC#N] | 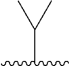 [SMILES: *C(C)C] | N |
| P27 | H | Br | 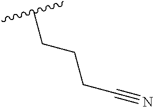 [SMILES: *CCCC#N] | 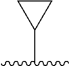 [SMILES: *C1CC1] | N |

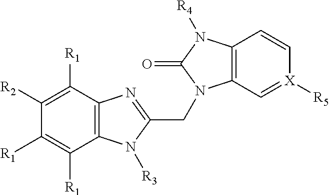

| | R₁ | R₂ | R₃ | R₄ | X—R₅ |
|---|---|---|---|---|---|
| P28 | H | Cl | 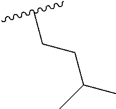 [SMILES: *CCC(C)C] | 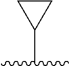 [SMILES: *C1CC1] | C—F |
| P29 | H | Cl | 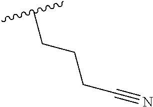 [SMILES: *CCCC#N] | 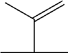 | N |
| P30 | H | Cl | 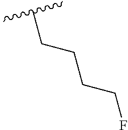 [SMILES: *CCCCF] | 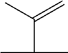 | N |
| P31 | H | Cl | 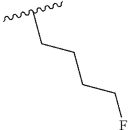 [SMILES: *CCCCF] | 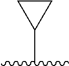 [SMILES: *C1CC1] | N |
| P32 | H | Br | 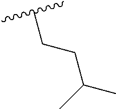 [SMILES: *CCC(C)C] | 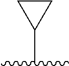 [SMILES: *C1CC1] | C—F |
| P33 | H | Cl | 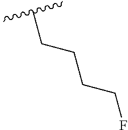 [SMILES: *CCCCF] | 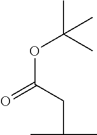 | N |
| P34 | H | Cl | 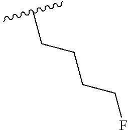 [SMILES: *CCCCF] | 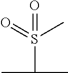 | N |
| P35 | H | Cl | 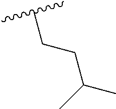 [SMILES: *CCC(C)C] | 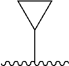 [SMILES: *C1CC1] | CH |
| P36 | H | Br | 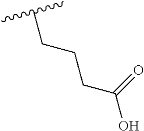 [SMILES: *CCCC(=O)O] | 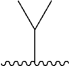 [SMILES: *C(C)C] | N |
| P37 | H | Cl | 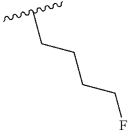 [SMILES: *CCCCF] | 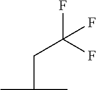 | N |
| P38 | H | Br | 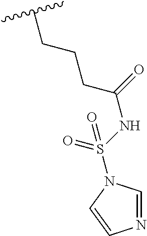 [SMILES: *CCCC(=O)NS(=O)(=O)n1ccnc1] | 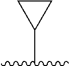 [SMILES: *C1CC1] | N |
| P39 | H | Cl | 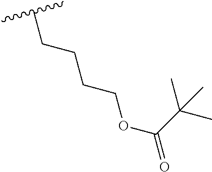 [SMILES: *CCCCOC(=O)C(C)(C)C] | 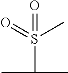 | N |
| | R₁ | R₂ | R₃ | R₄ | |
| P40 | H | Cl | 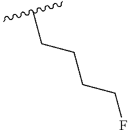 [SMILES: *CCCCF] | 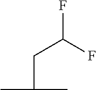 | |
| P41 | H | Cl | 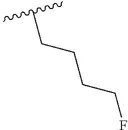 [SMILES: *CCCCF] | 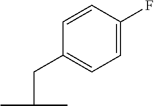 | |
| P42 | H | Cl | 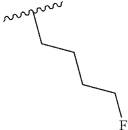 [SMILES: *CCCCF] | 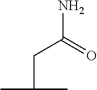 | |
| P43 | H | Cl | 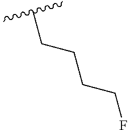 [SMILES: *CCCCF] | 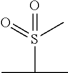 | |
| P44 | H | Cl | 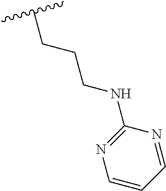 [SMILES: *CCCNc1ncccn1] | 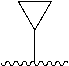 [SMILES: *C1CC1] | |
| P45 | H | Cl | 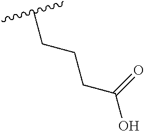 [SMILES: *CCCC(=O)O] | 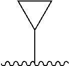 [SMILES: *C1CC1] | |
| P46 | H | Cl | 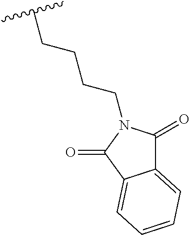 [SMILES: *CCCCN1C(=O)c2ccccc2C1=O] | 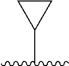 [SMILES: *C1CC1] | |
| P47 | H | Cl | 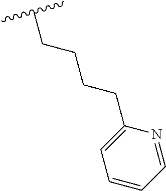 [SMILES: *CCCCc1ccccn1] | 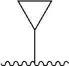 [SMILES: *C1CC1] | |
| P48 | H | Br | 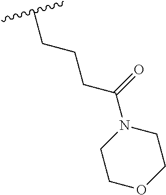 [SMILES: *CCCC(=O)N1CCOCC1] | 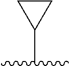 [SMILES: *C1CC1] | |
| P49 | H | Cl | 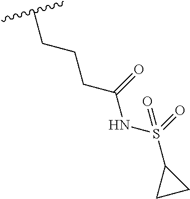 [SMILES: *CCCC(=O)NS(=O)(=O)C1CC1] | 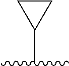 [SMILES: *C1CC1] | |
| P50 | H | Cl | 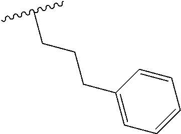 [SMILES: *CCCc1ccccc1] | 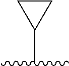 [SMILES: *C1CC1] | |
| P51 | H | Cl | 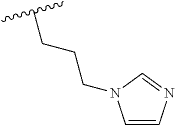 [SMILES: *CCCn1ccnc1] | 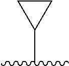 [SMILES: *C1CC1] | |
| | R₁ | R₂ | R₃ | R₄ | X—R₅ |
| P52 | H | Cl | 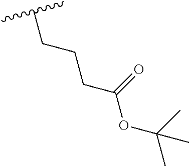 [SMILES: *CCCC(=O)OC(C)(C)C] | 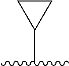 [SMILES: *C1CC1] | N |
| P53 | H | Cl | 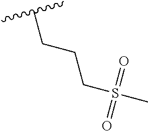 [SMILES: *CCCS(C)(=O)=O] | 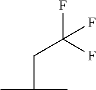 | C—F |
| P54 | H | Cl | 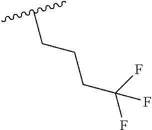 [SMILES: *CCCC(F)(F)F] | 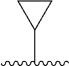 [SMILES: *C1CC1] | N |
| P55 | H | Cl | 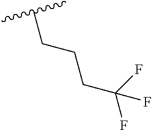 [SMILES: *CCCC(F)(F)F] | 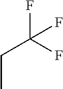 | N |
| P56 | H | Cl | 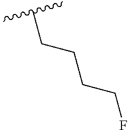 [SMILES: *CCCCF] | 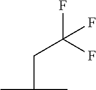 | C—F |
| P57 | H | Cl | 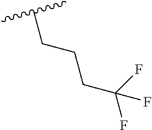 [SMILES: *CCCC(F)(F)F] | 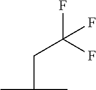 | C—F |
| P58 | H | Cl | 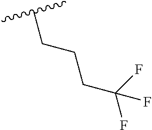 [SMILES: *CCCC(F)(F)F] | 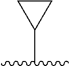 [SMILES: *C1CC1] | C—F |
| P59 | H | Cl | 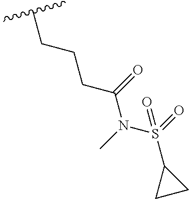 [SMILES: *CCCC(=O)N(C)S(=O)(=O)C1CC1] | 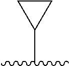 [SMILES: *C1CC1] | N |
| P60 | H | Cl | 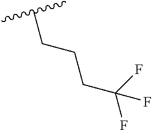 [SMILES: *CCCC(F)(F)F] | 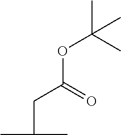 | N |
| P61 | H | Cl | 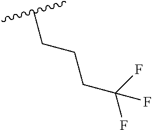 [SMILES: *CCCC(F)(F)F] | 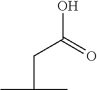 | N |
| P62 | H | Cl | 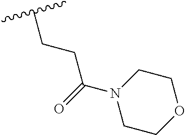 [SMILES: *CCC(=O)N1CCOCC1] | 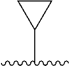 [SMILES: *C1CC1] | C—F |
| P63 | H | Cl | 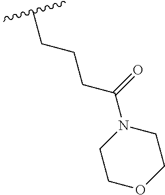 [SMILES: *CCCC(=O)N1CCOCC1] | 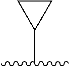 [SMILES: *C1CC1] | N |
| P64 | H | Cl | 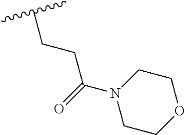 [SMILES: *CCC(=O)N1CCOCC1] | 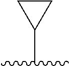 [SMILES: *C1CC1] | N |
| P65 | H | Cl | 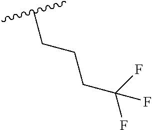 [SMILES: *CCCC(F)(F)F] | 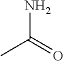 | N |
| P66 | H | Cl | 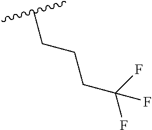 [SMILES: *CCCC(F)(F)F] | 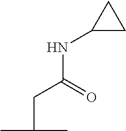 | N |
| P67 | H | Cl | 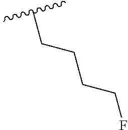 [SMILES: *CCCCF] | 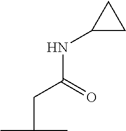 | N |
| P68 | H | Cl | 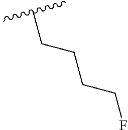 [SMILES: *CCCCF] | 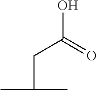 | N |
| P69 | H | Cl | 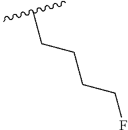 [SMILES: *CCCCF] | 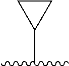 [SMILES: *C1CC1] | C—F |
| P70 | H | Cl | 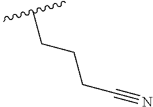 [SMILES: *CCCC#N] | 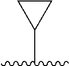 [SMILES: *C1CC1] | CH |
| P71 | H | Cl | 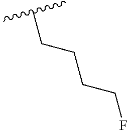 [SMILES: *CCCCF] | 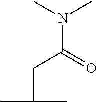 | N |
| P72 | H | Cl | 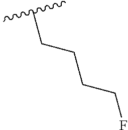 [SMILES: *CCCCF] | 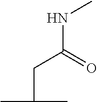 | N |
| P73 | H | Cl | 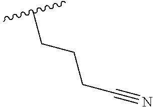 [SMILES: *CCCC#N] | 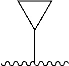 [SMILES: *C1CC1] | C—F |
| P74 | H | Br | 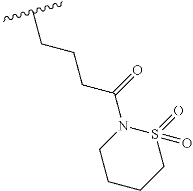 [SMILES: *CCCC(=O)N1CCCCS1(=O)=O] | 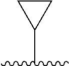 [SMILES: *C1CC1] | N |
| P75 | H | Br | 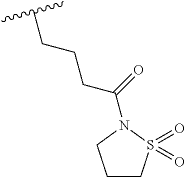 [SMILES: *CCCC(=O)N1CCCS1(=O)=O] | 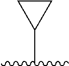 [SMILES: *C1CC1] | N |
| P76 | H | Br | 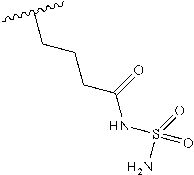 [SMILES: *CCCC(=O)NS(N)(=O)=O] | 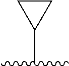 [SMILES: *C1CC1] | N |
| E. P77 | F. H | G. Br | 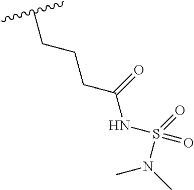 [SMILES: *CCCC(=O)NS(=O)(=O)N(C)C] | 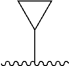 [SMILES: *C1CC1] | N |
| P78 | H | Br | 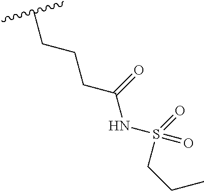 [SMILES: *CCCC(=O)NS(=O)(=O)CCC] | 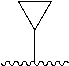 [SMILES: *C1CC1] | N |
| P79 | H | Br | 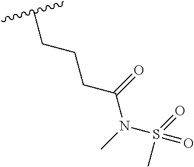 [SMILES: *CCCC(=O)N(C)S(C)(=O)=O] | 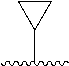 [SMILES: *C1CC1] | N |
| P80 | H | Br | 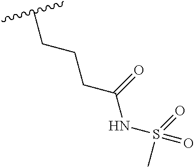 [SMILES: *CCCC(=O)NS(C)(=O)=O] | 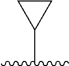 [SMILES: *C1CC1] | N |
| P81 | H | Br | 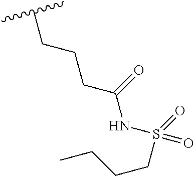 [SMILES: *CCCC(=O)NS(=O)(=O)CCCC] | 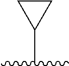 [SMILES: *C1CC1] | N |
| P82 | H | Br | 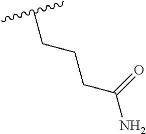 [SMILES: *CCCC(N)=O] | 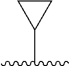 [SMILES: *C1CC1] | N |
| P83 | H | Br | 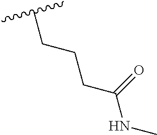 [SMILES: *CCCC(=O)NC] | 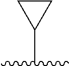 [SMILES: *C1CC1] | N |
| P84 | H | Cl | 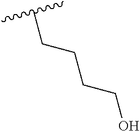 [SMILES: *CCCCO] | 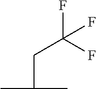 | N |
| P85 | H | Cl | 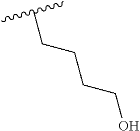 [SMILES: *CCCCO] | 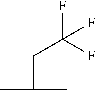 | C—F |

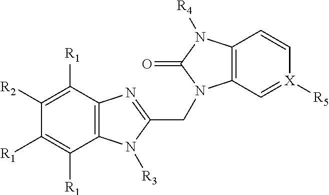

| | R₁ | R₂ | R₃ | R₄ | X—R₅ |
|---|---|---|---|---|---|
| P86 | H | Cl | 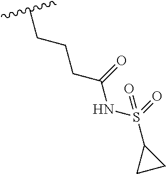 [SMILES: *CCCC(=O)NS(=O)(=O)C1CC1] | 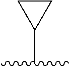 [SMILES: *C1CC1] | C—F |
| P87 | H | Cl | 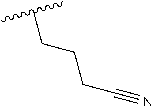 [SMILES: *CCCC#N] | 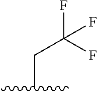 [SMILES: *CC(F)(F)F] | C—F |
| P88 | H | Cl | 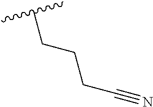 [SMILES: *CCCC#N] | 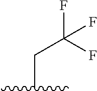 [SMILES: *CC(F)(F)F] | C—H |
| P89 | H | Cl | 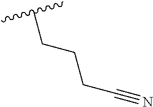 [SMILES: *CCCC#N] | 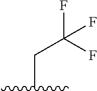 [SMILES: *CC(F)(F)F] | N |

### Compounds of Formula (B10)

Compounds of the general Formula (B10) are described in PCT Publication No. WO 2010/103306, published Sep. 16, 2010, which is hereby incorporated by reference in its entirety. Formula (B10) has the structure:

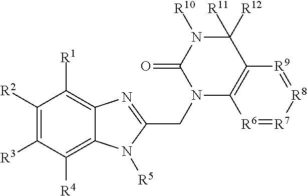

wherein: R¹, R³ and R⁴ each independently can represent H, C1-6 alkyl or halogen; R² can represent H, CN, CH₂NH₂, CH₂NH(CH₂)₃NH₂, C(═NH)NH₂ or C(═NOH)NH₂; R⁵ can represent C1-6 alkyl; said C1-6 alkyl being optionally substituted with one or more of OR¹³, CF₃, CN or NR¹⁴R¹⁵ wherein R¹³ can represent H or C1-6 alkyl and R¹⁴ and R¹⁵ independently can represent H, C1-6 alkyl or C3-7 cycloalkyl; or the group —NR¹⁴R¹⁵ together can represent a 5 to 7 membered azacyclic ring optionally incorporating one further heteroatom selected from O, S and NR¹⁹ wherein R¹⁹ can represent H or C1-6 alkyl; R⁶, R⁷, R⁸ and R⁹ each independently can represent CH, C—F, C—Cl, C—CF₃ or N; R¹⁹ can represent aryl, heteroaryl, C3-7 cycloalkyl or C1-6 alkyl; said C1-6 alkyl or C3-7 cycloalkyl being optionally substituted with one or more of aryl, C3-7 cycloalkyl, OR¹⁶, SR¹⁶, halogen or NR¹⁷R¹⁸, wherein R¹⁶ can represent H or C1-6 alkyl and R¹⁷ and R¹⁸ each independently can represent H, C1-6 alkyl or C3-7 cycloalkyl; or the group —NR¹⁷R¹ together represents a 5 to 7 membered azacyclic ring optionally incorporating one heteroatom selected from O, S and NR²⁹ wherein R²⁹ can represent H or C1-6 alkyl; and R¹¹ and R¹² each independently can represent H or C1-6 alkyl.

Examples of Compounds of Formula (B10) include: 3-methyl-1-[(1-isopentylbenzimidazol-2-yl)methyl]-4H-quinazolin-2-one; 3-isopentyl-1-[(1-isopentylbenzimidazol-2-yl)methyl]-4H-quinazolin-2-one; 3-cyclopropyl-1-[(1-isopentylbenzimidazol-2-yl)methyl]-4-methyl-4H-quinazolin-2-one; 3-cyclopropyl-1-[(1-isopentylbenzimidazol-2-yl)methyl]-4,4-dimethyl-quinazolin-2-one; 1-[[5-(aminomethyl)-1-isopentyl-benzimidazol-2-yl]methyl]-3-methyl-4H-quinazolin-2-one; 1-[[5-(aminomethyl)-1-isopentyl-benzimidazol-2-yl]methyl]-3-propyl-4H-quinazolin-2-one; 1-[[5-(aminomethyl)-1-isopentyl-benzimidazol-2-yl]methyl]-3-cyclopropyl-4H-quinazolin-2-one; 1-[[5-(aminomethyl)-1-isopentyl-benzimidazol-2-yl]methyl]-3-tert-butyl-4H-quinazolin-2-one; 1-[[5-(aminomethyl)-1-isopentyl-benzimidazol-2-yl]methyl]-3-cyclopentyl-4H-quinazolin-2-one; 1-[[5-(aminomethyl)-1-isopentyl-benzimidazol-2-yl]methyl]-3-benzyl-4H-quinazolin-2-one; 1-[[5-(aminomethyl)-1-isopentyl-benzimidazol-2-yl]methyl]-3-phenethyl-4H-quinazolin-2-one; 1-[[5-(aminomethyl)-1-isopentyl-benzimidazol-2-yl]methyl]-3-cyclopropyl-4H-pyrido[2,3-d]pyrimidin-2-one; 1-[[5-(aminomethyl)-1-isopentyl-benzimidazol-2-yl]methyl]-3-(2-methoxyethyl)-4H-quinazolin-2-one; 1-[[5-(aminomethyl)-1-isopentyl-benzimidazol-2-yl]methyl]-3-isopentyl-4H-quinazolin-2-one; 1-[[5-(aminomethyl)-1-isopentyl-benzimidazol-2-yl]methyl]-3-isobutyl-4H-quinazolin-2-one; 1-[[5-(aminomethyl)-1-isopentyl-benzimidazol-2-yl]methyl]-3-(cyclopropylmethyl)-4H-quinazolin-2-one; 1-[[5-(aminomethyl)-1-isopentyl-benzimidazol-2-yl]methyl]-3-(3-pyrrolidin-1-ylpropyl)-4H-quinazolin-2-one; 1-[[5-(aminomethyl)-1-isopentyl-benzimidazol-2-yl]methyl]-3-(2-methylsulfanylethyl)-4H-quinazolin-2-one; 1-[[5-(aminomethyl)-1-isopentyl-benzimidazol-2-yl]methyl]-3-(cyclo hexylmethyl)-4H-quinazolin-2-one; 1-[[5-(aminomethyl)-1-isopentyl-benzimidazol-2-yl]methyl]-3-cyclopropyl-4-methyl-4H-quinazolin-2-one; 1-[[5-(aminomethyl)-1-isopentyl-benzimidazol-2-yl]methyl]-3-cyclopropyl-4,4-dimethyl-quinazolin-2-one; 1-[[5-(aminomethyl)-1-isopentyl-benzimidazol-2-yl]methyl]-3-cyclopropyl-5-(trifluoromethyl)-4H-quinazolin-2-one; 1-[[5-(aminomethyl)-1-isopentyl-benzimidazol-2-yl]methyl]-3-cyclopropyl-5-fluoro-4H-quinazolin-2-one; 1-[[5-(aminomethyl)-1-(4-hydroxybutyl)benzimidazol-2-yl]methyl]-3-methyl-4H-quinazolin-2-one; 1-[[5-(aminomethyl)-1-(4-hydroxybutyl)benzimidazol-2-yl]methyl]-3-cyclopropyl-4H-quinazolin-2-one; 1-[[5-(aminomethyl)-1-(4-hydroxybutyl)benzimidazol-2-yl]methyl]-3-(2-methoxyethyl)-4H-quinazolin-2-one; 1-[[5-(aminomethyl)-1-(4-hydroxybutyl)benzimidazol-2-yl]methyl]-3-(cyclohexylmethyl)-4H-quinazolin-2-one; 1-[[5-(aminomethyl)-1-(4,4,4-trifluorobutyl)benzimidazol-2-yl]methyl]-3-cyclopropyl-4H-quinazolin-2-one; 1-[[5-(aminomethyl)-1-(4,4,4-trifluorobutyl)benzimidazol-2-yl]methyl]-3-cyclopropyl-4-methyl-4H-quinazolin-2-one; 1-[[5-(aminomethyl)-1-(4,4,4-trifluorobutyl)benzimidazol-2-yl]methyl]-3-cyclopropyl-4,4-dimethyl-quinazolin-2-one; 1-[[5-[(3-aminopropylamino)methyl]-1-isopentyl-benzimidazol-2-yl]methyl]-3-methyl-4H-quinazolin-2-one; 1-[[5-[(3-aminopropylamino)methyl]-1-isopentyl-benzimidazol-2-yl]methyl]-3-cyclopropyl-4H-quinazolin-2-one; 1-[[5-[(3-aminopropylamino)methyl]-1-isopentyl-benzimidazol-2-yl]methyl]-3-(2-methoxyethyl)-4H-quinazolin-2-one; 1-[[5-[(3-aminopropylamino)methyl]-1-(4-hydroxybutyl)benzimidazol-2-yl]methyl]-3-methyl-4H-quinazolin-2-one; 2-[(3-cyclopropyl-2-oxo-4H-quinazolin-1-yl)methyl]-1-isopentyl-benzimidazole-5-carboxamidine; 2-[(3-cyclopropyl-4-methyl-2-oxo-4H-quinazolin-1-yl)methyl]-1-isopentyl-benzimidazole-5-carboxamidine; 2-[(3-cyclopropyl-4,4-dimethyl-2-oxo-quinazolin-1-yl)methyl]-1-isopentyl-benzimidazole-5-carboxamidine; 2-[(3-cyclopropyl-2-oxo-4H-quinazolin-1-yl)methyl]-N′-hydroxy-1-isopentyl-benzimidazole-5-carboxamidine; 2-[(3-cyclopropyl-4-methyl-2-oxo-4H-quinazolin-1-yl)methyl]-N′-hydroxy-1-isopentyl-benzimidazole-5-carboxamidine; 2-[(3-cyclopropyl-4,4-dimethyl-2-oxo-quinazolin-1-yl)methyl]-N′-hydroxy-1-isopentyl-benzimidazole-5-carboxamidine; 2-[(3-cyclopropyl-2-oxo-4H-quinazolin-1-yl)methyl]-1-(4,4,4-trifluorobutyl)benzimidazole-5-carboxamidine; 2-[(3-cyclopropyl-4-methyl-2-oxo-4H-quinazolin-1-yl)methyl]-1-(4,4,4-trifluorobutyl)benzimidazole-5-carboxamidine; 2-[(3-cyclopropyl-4,4-dimethyl-2-oxo-quinazolin-1-yl)methyl]-1-(4,4,4-trifluorobutyl)benzimidazole-5-carboxamidine; 2-[(3-cyclopropyl-4-methyl-2-oxo-4H-quinazolin-1-yl)methyl]-N′-hydroxy-1-(4,4,4-trifluorobutyl)benzimidazole-5-carboxamidine; 2-[(3-cyclopropyl-4,4-dimethyl-2-oxo-quinazolin-1-yl)methyl]-N′-hydroxy-1-(4,4,4-trifluorobutyl)benzimidazole-5-carboxamidine; and 1-[[5-(aminomethyl)-1-isopentyl-6-methyl-benzimidazol-2-yl]methyl]-3-cyclopropyl-4H-quinazolin-2-one.

### Compounds of Formula (B11)

Compounds of the general Formula (B11) are described in PCT Publication No. WO 2012/068622, published May 31, 2012, which is hereby incorporated by reference in its entirety. Formula (B11) has the structure:

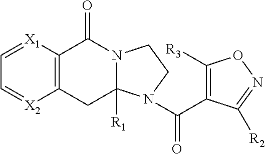

or racemates, isomers and/or salts thereof, wherein: X₁ and X₂ can be independently selected from CH and N wherein at least one of X₁ and X₂ is N; R₁ is optionally substituted and can be selected from a carbocyclic, heterocyclic and aromatic ring; R₂ can be selected from C₁₋₆ alkyl, haloC₁₋₃ alkyl and C₁₋₃alkoxy; and R₃ can be H or an optional substituent.

Examples of Compounds of Formula (B11) include: 5a-(4-chlorophenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; 10a-(4-chlorophenyl)-1-[(3-methyl-1,2-oxazol-4-yl)carbonyl]-2,3,10,10a-tetrahydroimidazo[2,1-g][1,7]naphthyridin-5 (1H)-one; 10a-(4-methoxyphenyl)-1-[(3-methyl-1,2-oxazol-4-yl)carbonyl]-2,3,10,10a-tetrahydroimidazo[2,1-g][1,7]naphthyridin-5 (1H)-one; 10a-(4-fluorophenyl)-1-[(3-methyl-1,2-oxazol-4-yl)carbonyl]-2,3,10,10a-tetrahydroimidazo[2,1-g][1,7]naphthyridin-5(1H)-one; 5a-(4-fluorophenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; 10a-(4-fluoro-3-methylphenyl)-1-[(3-methyl-1,2-oxazol-4-yl)carbonyl]-2,3,10,10a-tetrahydroimidazo[2,1-g][1,7]naphthyridin-5(1H)-one; 10a-(3,4-difluorophenyl)-1-[(3-methyl-1,2-oxazol-4-yl)carbonyl]-2,3,10,10a-tetrahydroimidazo[2,1-g][1,7]naphthyridin-5(1H)-one; 5a-(3,4-difluorophenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; 5a-(4-fluoro-3-methylphenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; 10a-(2-chlorophenyl)-1-[(3-methyl-1,2-oxazol-4-yl)carbonyl]-2,3,10,10a-tetrahydroimidazo[2,1-g][1,7]naphthyridin-5(1H)-one; 10a-cyclo hexyl-1-[(3-methyl-1,2-oxazol-4-yl)carbonyl]-2,3,10,10a-tetrahydroimidazo[2,1-g][1,7]naphthyridin-5(1H)-one; 10a-(4,4-difluorocyclohexyl)-1-[(3-methyl-1,2-oxazol-4-yl)carbonyl]-2,3,10,10a-tetrahydroimidazo[2,1-g][1,7]naphthyridin-5 (1H)-one; 10a-(4-chlorophenyl)-1-{[3-(trifluoromethyl)-1,2-oxazol-4-yl]carbonyl}-2,3,10,10a-tetrahydroimidazo[2,1-g][1,7]naphthyridin-5(1H)-one; 10a-(2,3-dihydro-1-benzofuran-5-yl)-1-[(3-methyl-1,2-oxazol-4-yl)carbonyl]-2,3,10,10a-tetrahydroimidazo[2,1-g][1,7]naphthyridin-5 (1H)-one; (5aS)-5a-(4-chlorophenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; (10aS)-10a-(4-chlorophenyl)-1-[(3-methyl-1,2-oxazol-4-yl)carbonyl]-2,3,10,10a-tetrahydroimidazo[2,1-g][1,7]naphthyridin-5 (1H)-one; (10aS)-10a-(4-fluorophenyl)-1-[(3-methyl-1,2-oxazol-4-yl)carbonyl]-2,3,10,10a-tetrahydroimidazo[2,1-g][1,7]naphthyridin-5 (1H)-one; (5aS)-5a-(4-fluorophenyl)-6-[(3-methyl-1,2-oxazol-4-yl)carbonyl]-5a,6,7,8-tetrahydroimidazo[1′,2′:1,6]pyrido[3,4-b]pyrazin-10(5H)-one; and (10aS)-10a-(4-fluoro-3-methylphenyl)-1-[(3-methyl-1,2-oxazol-4-yl)carbonyl]-2,3,10,10a-tetrahydroimidazo[2,1-g][1,7]naphthyridin-5 (1H)-one.

### Compounds of Formula (B12)

Compounds of the general Formula (B12) are described in PCT Publication No. WO 2005/042530, published May 12, 2005, which is hereby incorporated by reference in its entirety. Formula (B12) has the structure:

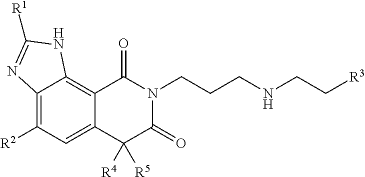

or an enantiomer or a salt thereof, wherein: R¹ can be —(CH═CH)₀₋₁—(C₆ or C₁₀)aryl or —(CH═CH)₀₋₁-5-, 6-, 9- or 10-membered heteroaryl, said aryl or heteroaryl being optionally substituted with one, two or three substituents, each independently selected from: (C₁₋₆)alkyl optionally substituted with amino, halo, C₁₋₆)haloalkyl, hydroxy, C₁₋₆)alkoxy, C₁₋₆)alkylthio, nitro, azido, cyano, amino, (C₁₋₆)alkylamino, di(C₁₋₆)alkyl)amino, aryl and heteroaryl; R² can be H, C₁₋₆)alkyl, hydroxy, halo, C₁₋₆)haloalkyl, amino, C₁₋₆)alkylamino. di(C₁₋₆)alkyl)amino, or (C₂₋₆)alkynyl; R³ can be (C₆, C₁₀ or C₁₄)aryl or 5-, 6-, 9- or 10-membered heteroaryl, each of which being optionally substituted with one, two or three substituents, each independently selected from: (C₁₋₆)alkyl, halo, C₁₋₆)haloalkyl, hydroxy, C₁₋₆)alkoxy, C₁₋₆)alkylthio, nitro, amino, C₁₋₆)alkylamino, di(C₁₋₆)alkyl)amino and COO(C₁₋₆)alkyl; and R⁴ and R⁵ can be each independently H or (C₁₋₆)alkyl; or R⁴ and R⁵ can be linked, together with the carbon atom to which they are attached, to form a (C₃₋₇)cycloalkyl group; with the proviso that R¹ is not 2-methoxyphenyl, when R² is H, R³ is 3,4-dimethoxyphenyl, R⁴ is CH₃ and R⁵ is CH₃.

Examples of Compounds of Formula (B12) include:

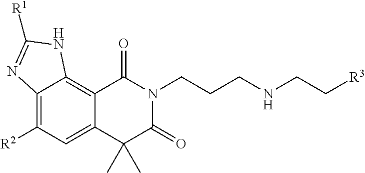

| Cpd | | | |
|---|---|---|---|
| entry | | | |
| # | R¹ | R² | R³ |
| 1002 | 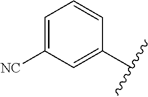 [SMILES: *c1cccc(C#N)c1] | H | 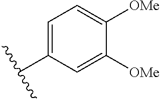 [SMILES: *c1ccc(OC)c(OC)c1] |
| 1003 | 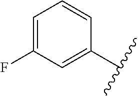 [SMILES: *c1cccc(F)c1] | H | 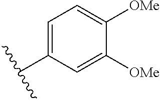 [SMILES: *c1ccc(OC)c(OC)c1] |
| 1004 | 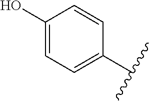 [SMILES: *c1ccc(O)cc1] | H | 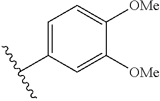 [SMILES: *c1ccc(OC)c(OC)c1] |
| 1005 | 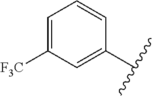 [SMILES: *c1cccc(C(F)(F)F)c1] | H | 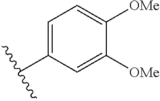 [SMILES: *c1ccc(OC)c(OC)c1] |
| 1006 | 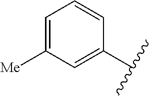 [SMILES: *c1cccc(C)c1] | H | 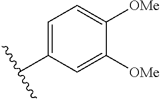 [SMILES: *c1ccc(OC)c(OC)c1] |
| 1007 | 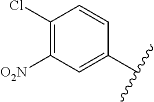 [SMILES: *c1ccc(Cl)c([N+](=O)[O-])c1] | H | 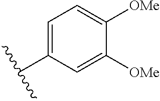 [SMILES: *c1ccc(OC)c(OC)c1] |
| 1008 | 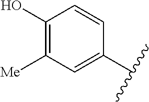 [SMILES: *c1ccc(O)c(C)c1] | H | 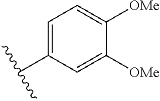 [SMILES: *c1ccc(OC)c(OC)c1] |
| 1009 | 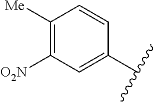 [SMILES: *c1ccc(C)c([N+](=O)[O-])c1] | H | 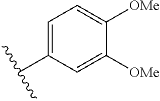 [SMILES: *c1ccc(OC)c(OC)c1] |
| 1010 | 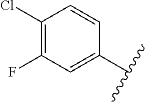 [SMILES: *c1ccc(Cl)c(F)c1] | H | 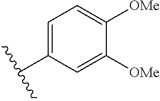 [SMILES: *c1ccc(OC)c(OC)c1] |
| 1011 | 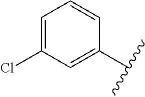 [SMILES: *c1cccc(Cl)c1] | H | 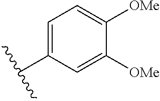 [SMILES: *c1ccc(OC)c(OC)c1] |
| 1012 | 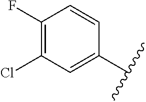 [SMILES: *c1ccc(F)c(Cl)c1] | H | 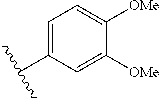 [SMILES: *c1ccc(OC)c(OC)c1] |
| 1013 | 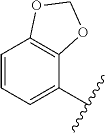 [SMILES: *c1cccc2c1OCO2] | H | 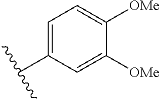 [SMILES: *c1ccc(OC)c(OC)c1] |
| 1014 | 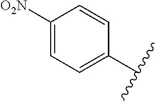 [SMILES: *c1ccc([N+](=O)[O-])cc1] | H | 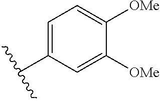 [SMILES: *c1ccc(OC)c(OC)c1] |
| 1015 | 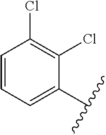 [SMILES: *c1cccc(Cl)c1Cl] | H | 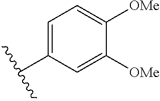 [SMILES: *c1ccc(OC)c(OC)c1] |
| 1016 | 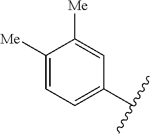 [SMILES: *c1ccc(C)c(C)c1] | H | 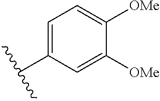 [SMILES: *c1ccc(OC)c(OC)c1] |
| 1017 | 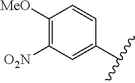 [SMILES: *c1ccc(OC)c([N+](=O)[O-])c1] | H | 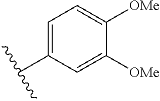 [SMILES: *c1ccc(OC)c(OC)c1] |
| 1018 | 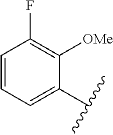 [SMILES: *c1cccc(F)c1OC] | H | 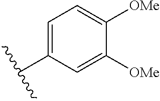 [SMILES: *c1ccc(OC)c(OC)c1] |
| 1019 | 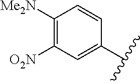 [SMILES: *c1ccc(N(C)C)c([N+](=O)[O-])c1] | H | 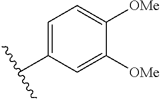 [SMILES: *c1ccc(OC)c(OC)c1] |
| 1020 | 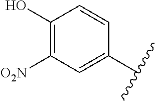 [SMILES: *c1ccc(O)c([N+](=O)[O-])c1] | H | 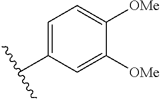 [SMILES: *c1ccc(OC)c(OC)c1] |
| 1021 | 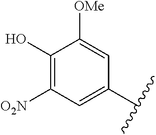 [SMILES: *c1cc(OC)c(O)c([N+](=O)[O-])c1] | H | 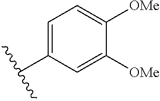 [SMILES: *c1ccc(OC)c(OC)c1] |
| 1022 | 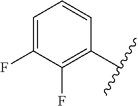 [SMILES: *c1cccc(F)c1F] | H | 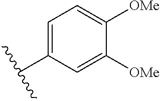 [SMILES: *c1ccc(OC)c(OC)c1] |
| 1023 | 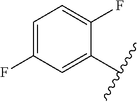 [SMILES: *c1cc(F)ccc1F] | H | 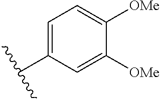 [SMILES: *c1ccc(OC)c(OC)c1] |
| 1024 | 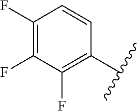 [SMILES: *c1ccc(F)c(F)c1F] | H | 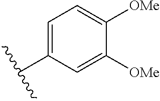 [SMILES: *c1ccc(OC)c(OC)c1] |
| 1025 | 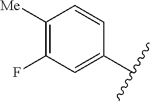 [SMILES: *c1ccc(C)c(F)c1] | H | 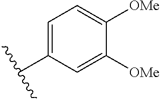 [SMILES: *c1ccc(OC)c(OC)c1] |
| 1026 | 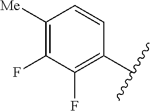 [SMILES: *c1ccc(C)c(F)c1F] | H | 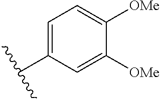 [SMILES: *c1ccc(OC)c(OC)c1] |
| 1027 | 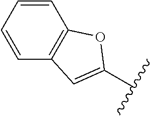 [SMILES: *c1cc2ccccc2o1] | H | 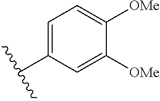 [SMILES: *c1ccc(OC)c(OC)c1] |
| 1028 | 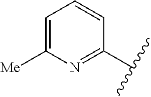 [SMILES: *c1cccc(C)n1] | H | 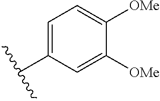 [SMILES: *c1ccc(OC)c(OC)c1] |
| 1029 | 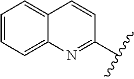 [SMILES: *c1ccc2ccccc2n1] | H | 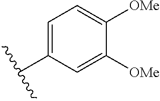 [SMILES: *c1ccc(OC)c(OC)c1] |
| 1030 | 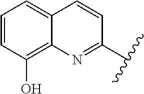 [SMILES: *c1ccc2cccc(O)c2n1] | H | 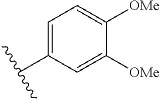 [SMILES: *c1ccc(OC)c(OC)c1] |
| 1031 | 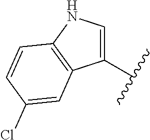 [SMILES: *c1c[nH]c2ccc(Cl)cc12] | H | 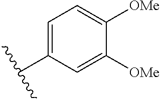 [SMILES: *c1ccc(OC)c(OC)c1] |
| 1032 | 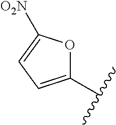 [SMILES: *c1ccc([N+](=O)[O-])o1] | H | 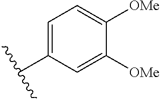 [SMILES: *c1ccc(OC)c(OC)c1] |
| 1033 | 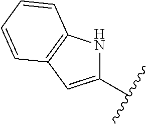 [SMILES: *c1cc2ccccc2[nH]1] | H | 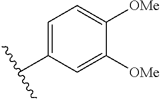 [SMILES: *c1ccc(OC)c(OC)c1] |
| 1034 | 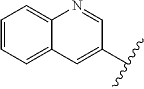 [SMILES: *c1cnc2ccccc2c1] | H | 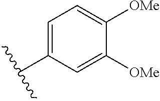 [SMILES: *c1ccc(OC)c(OC)c1] |
| 1035 | 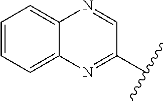 [SMILES: *c1cnc2ccccc2n1] | H | 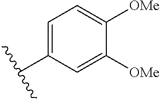 [SMILES: *c1ccc(OC)c(OC)c1] |
| 1036 | 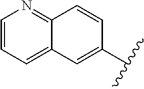 [SMILES: *c1ccc2ncccc2c1] | H | 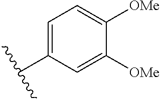 [SMILES: *c1ccc(OC)c(OC)c1] |
| 1037 | 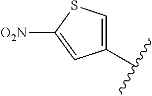 [SMILES: *c1csc([N+](=O)[O-])c1] | H | 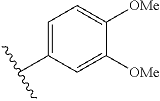 [SMILES: *c1ccc(OC)c(OC)c1] |
| 1038 | 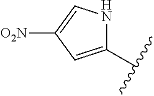 [SMILES: *c1cc([N+](=O)[O-])c[nH]1] | H | 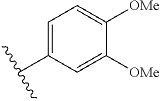 [SMILES: *c1ccc(OC)c(OC)c1] |
| 1039 | 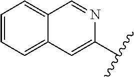 [SMILES: *c1cc2ccccc2cn1] | H | 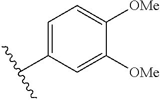 [SMILES: *c1ccc(OC)c(OC)c1] |
| 1040 | 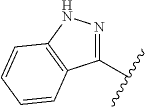 [SMILES: *c1n[nH]c2ccccc12] | H | 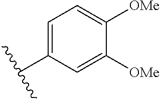 [SMILES: *c1ccc(OC)c(OC)c1] |
| 1041 | 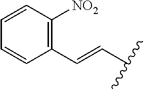 [SMILES: */C=C/c1ccccc1[N+](=O)[O-]] | H | 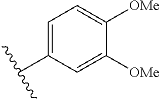 [SMILES: *c1ccc(OC)c(OC)c1] |
| 1042 | 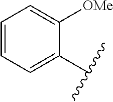 [SMILES: *c1ccccc1OC] | H | 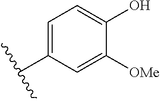 [SMILES: *c1ccc(O)c(OC)c1] |
| 1043 | 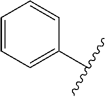 [SMILES: *c1ccccc1] | H | 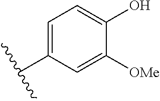 [SMILES: *c1ccc(O)c(OC)c1] |
| 1044 | 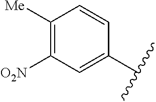 [SMILES: *c1ccc(C)c([N+](=O)[O-])c1] | H | 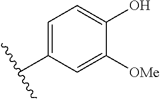 [SMILES: *c1ccc(O)c(OC)c1] |
| 1045 | 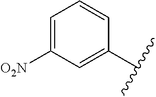 [SMILES: *c1cccc([N+](=O)[O-])c1] | H | 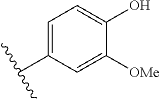 [SMILES: *c1ccc(O)c(OC)c1] |
| 1046 | 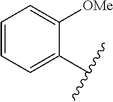 [SMILES: *c1ccccc1OC] | Br | 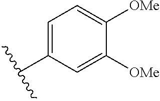 [SMILES: *c1ccc(OC)c(OC)c1] |
| 1047 | 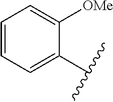 [SMILES: *c1ccccc1OC] | Et | 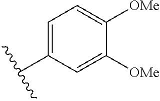 [SMILES: *c1ccc(OC)c(OC)c1] |
| 1048 | 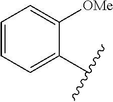 [SMILES: *c1ccccc1OC] | NH₂ | 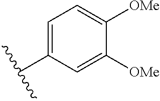 [SMILES: *c1ccc(OC)c(OC)c1] |
| 1049 | 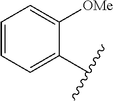 [SMILES: *c1ccccc1OC] | C≡CH | 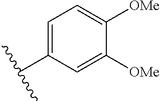 [SMILES: *c1ccc(OC)c(OC)c1] |
| 1050 | 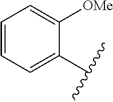 [SMILES: *c1ccccc1OC] | NHMe | 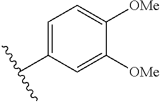 [SMILES: *c1ccc(OC)c(OC)c1] |
| 1051 | 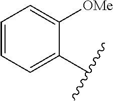 [SMILES: *c1ccccc1OC] | Me | 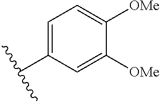 [SMILES: *c1ccc(OC)c(OC)c1] |
| 1052 | 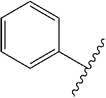 [SMILES: *c1ccccc1] | Br | 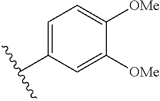 [SMILES: *c1ccc(OC)c(OC)c1] |
| 1053 | 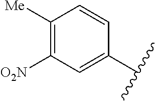 [SMILES: *c1ccc(C)c([N+](=O)[O-])c1] | Br | 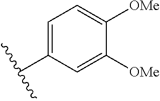 [SMILES: *c1ccc(OC)c(OC)c1] |
| 1054 | 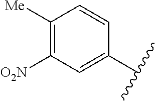 [SMILES: *c1ccc(C)c([N+](=O)[O-])c1] | Cl | 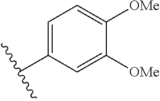 [SMILES: *c1ccc(OC)c(OC)c1] |
| 1055 | 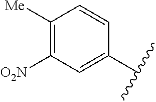 [SMILES: *c1ccc(C)c([N+](=O)[O-])c1] | OH | 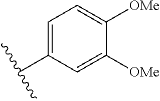 [SMILES: *c1ccc(OC)c(OC)c1] |
| 1056 | 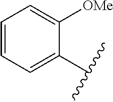 [SMILES: *c1ccccc1OC] | Br | 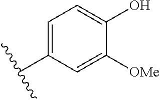 [SMILES: *c1ccc(O)c(OC)c1] |
| 1057 | 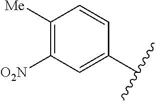 [SMILES: *c1ccc(C)c([N+](=O)[O-])c1] | Br | 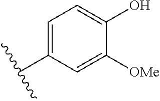 [SMILES: *c1ccc(O)c(OC)c1] |
| 1058 | 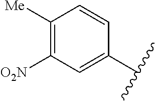 [SMILES: *c1ccc(C)c([N+](=O)[O-])c1] | NH₂ | 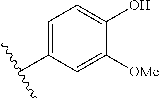 [SMILES: *c1ccc(O)c(OC)c1] |
| 1059 | 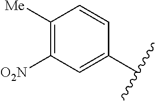 [SMILES: *c1ccc(C)c([N+](=O)[O-])c1] | NH₂ | 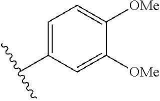 [SMILES: *c1ccc(OC)c(OC)c1] |
| 1060 | 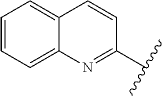 [SMILES: *c1ccc2ccccc2n1] | Br | 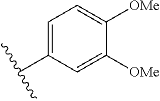 [SMILES: *c1ccc(OC)c(OC)c1] |
| 1061 | 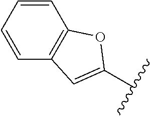 [SMILES: *c1cc2ccccc2o1] | Br | 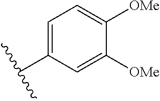 [SMILES: *c1ccc(OC)c(OC)c1] |
| 1062 | 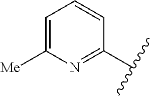 [SMILES: *c1cccc(C)n1] | Br | 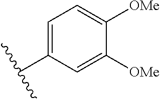 [SMILES: *c1ccc(OC)c(OC)c1] |
| 1063 | 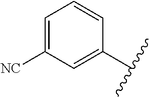 [SMILES: *c1cccc(C#N)c1] | Br | 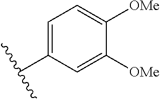 [SMILES: *c1ccc(OC)c(OC)c1] |
| 1064 | 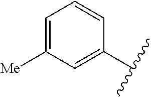 [SMILES: *c1cccc(C)c1] | Br | 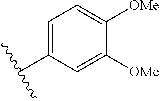 [SMILES: *c1ccc(OC)c(OC)c1] |
| 1065 | 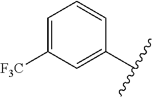 [SMILES: *c1cccc(C(F)(F)F)c1] | Br | 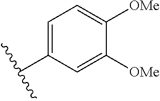 [SMILES: *c1ccc(OC)c(OC)c1] |
| 1066 | 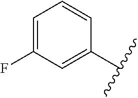 [SMILES: *c1cccc(F)c1] | Br | 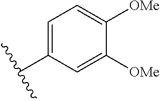 [SMILES: *c1ccc(OC)c(OC)c1] |
| 1067 | 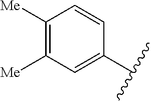 [SMILES: *c1ccc(C)c(C)c1] | Br | 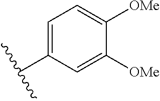 [SMILES: *c1ccc(OC)c(OC)c1] |
| 1068 | 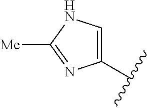 [SMILES: *c1c[nH]c(C)n1] | Br | 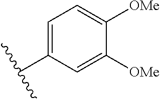 [SMILES: *c1ccc(OC)c(OC)c1] |
| 1069 | 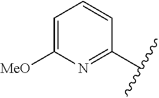 [SMILES: *c1cccc(OC)n1] | Br | 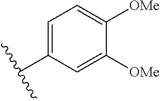 [SMILES: *c1ccc(OC)c(OC)c1] |
| 1070 | 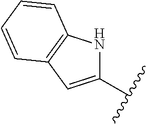 [SMILES: *c1cc2ccccc2[nH]1] | Br | 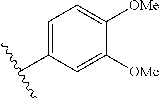 [SMILES: *c1ccc(OC)c(OC)c1] |
| 1071 | 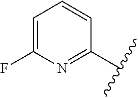 [SMILES: *c1cccc(F)n1] | Br | 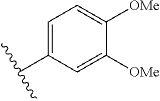 [SMILES: *c1ccc(OC)c(OC)c1] |
| 1072 | 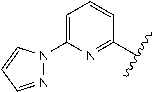 [SMILES: *c1cccc(-n2cccn2)n1] | Br | 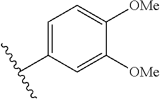 [SMILES: *c1ccc(OC)c(OC)c1] |
| 1073 | 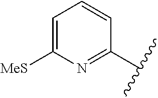 [SMILES: *c1cccc(SC)n1] | Br | 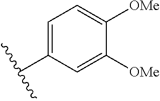 [SMILES: *c1ccc(OC)c(OC)c1] |
| 1074 | 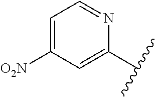 [SMILES: *c1cc([N+](=O)[O-])ccn1] | Br | 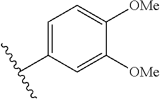 [SMILES: *c1ccc(OC)c(OC)c1] |
| 1075 | 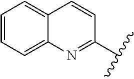 [SMILES: *c1ccc2ccccc2n1] | NH₂ | 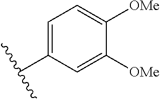 [SMILES: *c1ccc(OC)c(OC)c1] |
| 1076 | 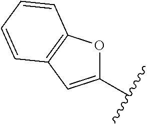 [SMILES: *c1cc2ccccc2o1] | NH₂ | 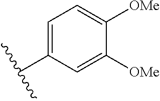 [SMILES: *c1ccc(OC)c(OC)c1] |
| 1077 | 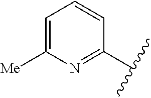 [SMILES: *c1cccc(C)n1] | NH₂ | 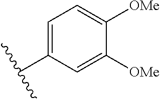 [SMILES: *c1ccc(OC)c(OC)c1] |
| 1078 | 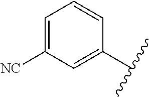 [SMILES: *c1cccc(C#N)c1] | NH₂ | 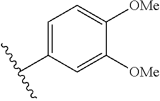 [SMILES: *c1ccc(OC)c(OC)c1] |
| 1079 | 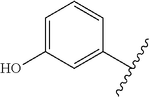 [SMILES: *c1cccc(O)c1] | NH₂ | 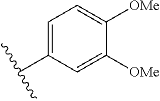 [SMILES: *c1ccc(OC)c(OC)c1] |
| 1080 | 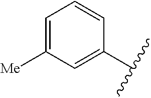 [SMILES: *c1cccc(C)c1] | NH₂ | 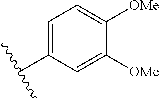 [SMILES: *c1ccc(OC)c(OC)c1] |
| 1081 | 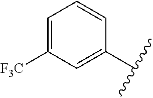 [SMILES: *c1cccc(C(F)(F)F)c1] | NH₂ | 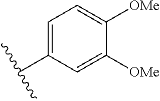 [SMILES: *c1ccc(OC)c(OC)c1] |
| 1082 | 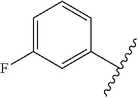 [SMILES: *c1cccc(F)c1] | NH₂ | 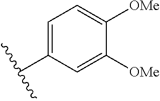 [SMILES: *c1ccc(OC)c(OC)c1] |
| 1083 | 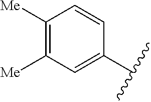 [SMILES: *c1ccc(C)c(C)c1] | NH₂ | 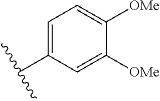 [SMILES: *c1ccc(OC)c(OC)c1] |
| 1084 | 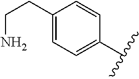 [SMILES: *c1ccc(CCN)cc1] | NH₂ | 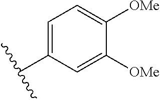 [SMILES: *c1ccc(OC)c(OC)c1] |
| 1085 | 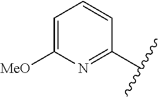 [SMILES: *c1cccc(OC)n1] | NH₂ | 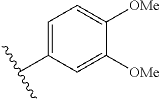 [SMILES: *c1ccc(OC)c(OC)c1] |
| 1086 | 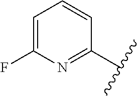 [SMILES: *c1cccc(F)n1] | NH₂ | 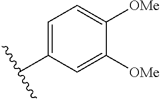 [SMILES: *c1ccc(OC)c(OC)c1] |
| 1087 | 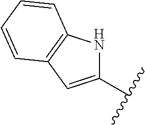 [SMILES: *c1cc2ccccc2[nH]1] | NH₂ | 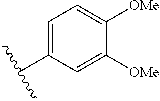 [SMILES: *c1ccc(OC)c(OC)c1] |
| 1088 | 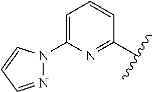 [SMILES: *c1cccc(-n2cccn2)n1] | NH₂ | 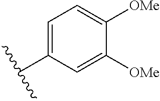 [SMILES: *c1ccc(OC)c(OC)c1] |
| 1089 | 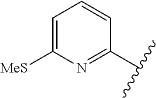 [SMILES: *c1cccc(SC)n1] | NH₂ | 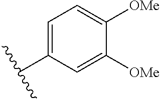 [SMILES: *c1ccc(OC)c(OC)c1] |
| 1090 | 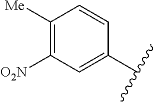 [SMILES: *c1ccc(C)c([N+](=O)[O-])c1] | Br | 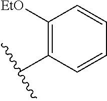 [SMILES: *c1ccccc1OCC] |
| 1091 | 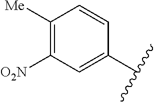 [SMILES: *c1ccc(C)c([N+](=O)[O-])c1] | Br | 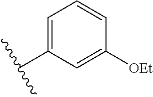 [SMILES: *c1cccc(OCC)c1] |
| 1092 | 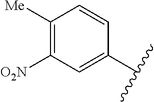 [SMILES: *c1ccc(C)c([N+](=O)[O-])c1] | Br | 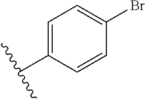 [SMILES: *c1ccc(Br)cc1] |
| 1093 | 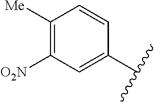 [SMILES: *c1ccc(C)c([N+](=O)[O-])c1] | Br | 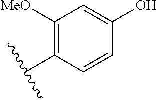 [SMILES: *c1ccc(O)cc1OC] |
| 1094 | 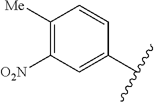 [SMILES: *c1ccc(C)c([N+](=O)[O-])c1] | Br | 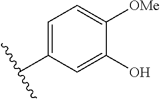 [SMILES: *c1ccc(OC)c(O)c1] |
| 1095 | 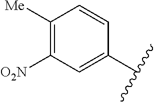 [SMILES: *c1ccc(C)c([N+](=O)[O-])c1] | Br | 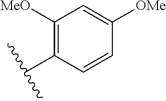 [SMILES: *c1ccc(OC)cc1OC] |
| 1096 | 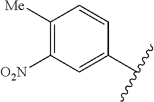 [SMILES: *c1ccc(C)c([N+](=O)[O-])c1] | Br | 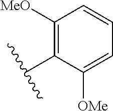 [SMILES: *c1c(OC)cccc1OC] |
| 1097 | 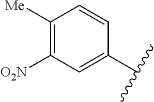 [SMILES: *c1ccc(C)c([N+](=O)[O-])c1] | Br | 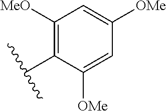 [SMILES: *c1c(OC)cc(OC)cc1OC] |
| 1098 | 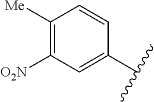 [SMILES: *c1ccc(C)c([N+](=O)[O-])c1] | Br | 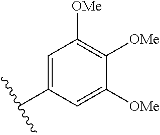 [SMILES: *c1cc(OC)c(OC)c(OC)c1] |
| 1099 | 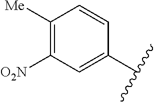 [SMILES: *c1ccc(C)c([N+](=O)[O-])c1] | Br | 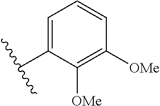 [SMILES: *c1cccc(OC)c1OC] |
| 1100 | 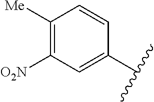 [SMILES: *c1ccc(C)c([N+](=O)[O-])c1] | Br | 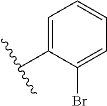 [SMILES: *c1ccccc1Br] |
| 1101 | 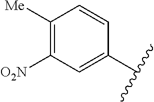 [SMILES: *c1ccc(C)c([N+](=O)[O-])c1] | Br | 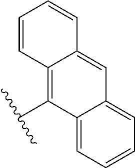 [SMILES: *c1c2ccccc2cc2ccccc12] |
| 1102 | 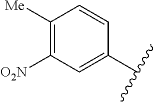 [SMILES: *c1ccc(C)c([N+](=O)[O-])c1] | Br | 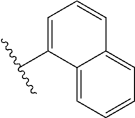 [SMILES: *c1cccc2ccccc12] |
| 1103 | 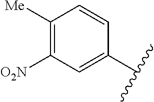 [SMILES: *c1ccc(C)c([N+](=O)[O-])c1] | Br | 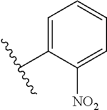 [SMILES: *c1ccccc1[N+](=O)[O-]] |
| 1104 | 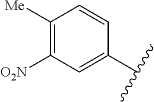 [SMILES: *c1ccc(C)c([N+](=O)[O-])c1] | Br | 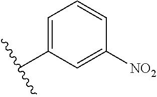 [SMILES: *c1cccc([N+](=O)[O-])c1] |
| 1105 | 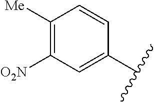 [SMILES: *c1ccc(C)c([N+](=O)[O-])c1] | Br | 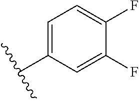 [SMILES: *c1ccc(F)c(F)c1] |
| 1106 | 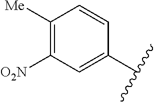 [SMILES: *c1ccc(C)c([N+](=O)[O-])c1] | Br | 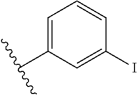 [SMILES: *c1cccc(I)c1] |
| 1107 | 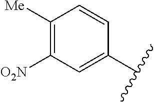 [SMILES: *c1ccc(C)c([N+](=O)[O-])c1] | Br | 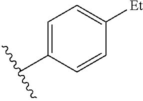 [SMILES: *c1ccc(CC)cc1] |
| 1108 | 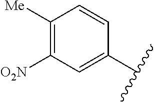 [SMILES: *c1ccc(C)c([N+](=O)[O-])c1] | Br | 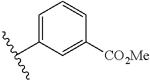 [SMILES: *c1cccc(C(=O)OC)c1] |
| 1109 | 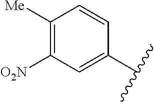 [SMILES: *c1ccc(C)c([N+](=O)[O-])c1] | Br | 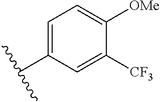 [SMILES: *c1ccc(OC)c(C(F)(F)F)c1] |
| 1110 | 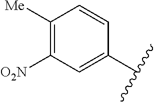 [SMILES: *c1ccc(C)c([N+](=O)[O-])c1] | Br | 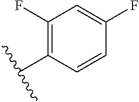 [SMILES: *c1ccc(F)cc1F] |
| 1111 | 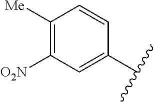 [SMILES: *c1ccc(C)c([N+](=O)[O-])c1] | Br | 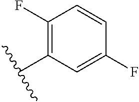 [SMILES: *c1cc(F)ccc1F] |
| 1112 | 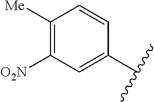 [SMILES: *c1ccc(C)c([N+](=O)[O-])c1] | Br | 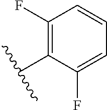 [SMILES: *c1c(F)cccc1F] |
| 1113 | 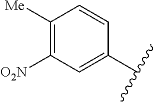 [SMILES: *c1ccc(C)c([N+](=O)[O-])c1] | Br | 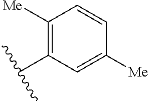 [SMILES: *c1cc(C)ccc1C] |
| 1114 | 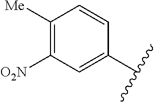 [SMILES: *c1ccc(C)c([N+](=O)[O-])c1] | Br | 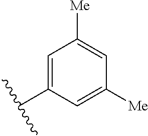 [SMILES: *c1cc(C)cc(C)c1] |
| 1115 | 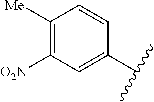 [SMILES: *c1ccc(C)c([N+](=O)[O-])c1] | Br | 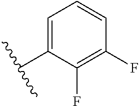 [SMILES: *c1cccc(F)c1F] |
| 1116 | 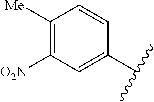 [SMILES: *c1ccc(C)c([N+](=O)[O-])c1] | Br | 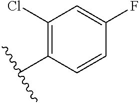 [SMILES: *c1ccc(F)cc1Cl] |
| 1117 | 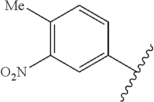 [SMILES: *c1ccc(C)c([N+](=O)[O-])c1] | Br | 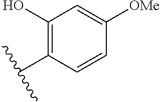 [SMILES: *c1ccc(OC)cc1O] |
| 1118 | 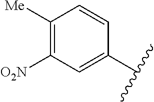 [SMILES: *c1ccc(C)c([N+](=O)[O-])c1] | Br | 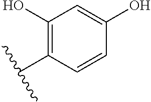 [SMILES: *c1ccc(O)cc1O] |
| 1129 | 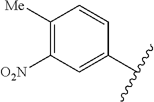 [SMILES: *c1ccc(C)c([N+](=O)[O-])c1] | Br | 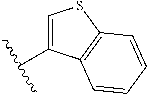 [SMILES: *c1csc2ccccc12] |
| 1130 | 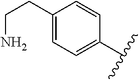 [SMILES: *c1ccc(CCN)cc1] | NH₂ | 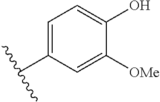 [SMILES: *c1ccc(O)c(OC)c1] |
| 1131 | 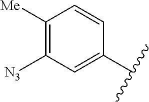 [SMILES: *c1ccc(C)c(N=[N+]=[N-])c1] | NH₂ | 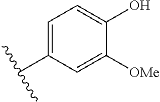 [SMILES: *c1ccc(O)c(OC)c1] |
| 1132 | 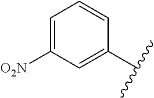 [SMILES: *c1cccc([N+](=O)[O-])c1] | H | 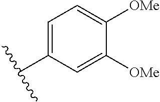 [SMILES: *c1ccc(OC)c(OC)c1] |

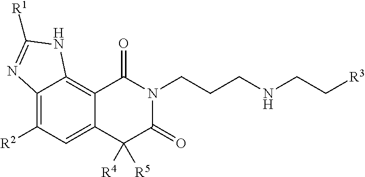

| Cpd entry # | R¹ | R² | 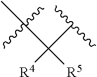 | R³ |
|---|---|---|---|---|
| 2001 | 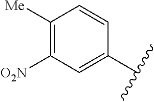 [SMILES: *c1ccc(C)c([N+](=O)[O-])c1] | H | 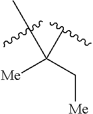 [SMILES: *C(*)(C)CC] | 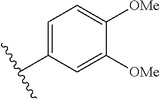 [SMILES: *c1ccc(OC)c(OC)c1] |
| 2002 | 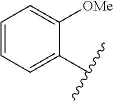 [SMILES: *c1ccccc1OC] | H | 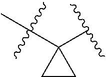 [SMILES: *C1(*)CC1] | 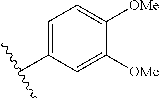 [SMILES: *c1ccc(OC)c(OC)c1] |
| 2003 | 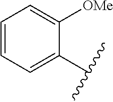 [SMILES: *c1ccccc1OC] | H | 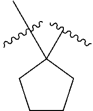 [SMILES: *C1(*)CCCC1] | 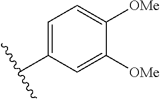 [SMILES: *c1ccc(OC)c(OC)c1] |
| 2004 | 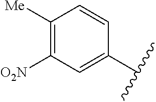 [SMILES: *c1ccc(C)c([N+](=O)[O-])c1] | H | 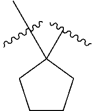 [SMILES: *C1(*)CCCC1] | 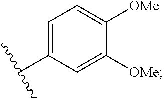 [SMILES: *c1ccc(OC)c(OC)c1] |
| 2005 | 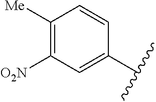 [SMILES: *c1ccc(C)c([N+](=O)[O-])c1] | Br | 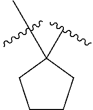 [SMILES: *C1(*)CCCC1] | 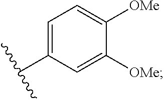 [SMILES: *c1ccc(OC)c(OC)c1] |
| 2006 | 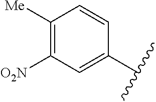 [SMILES: *c1ccc(C)c([N+](=O)[O-])c1] | NH₂ | 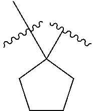 [SMILES: *C1(*)CCCC1] | 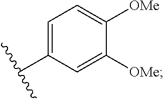 [SMILES: *c1ccc(OC)c(OC)c1] |
| 2007 | 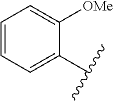 [SMILES: *c1ccccc1OC] | H | 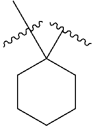 [SMILES: *C1(*)CCCCC1] | 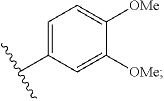 [SMILES: *c1ccc(OC)c(OC)c1] |
| 2008 | 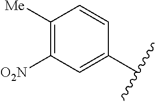 [SMILES: *c1ccc(C)c([N+](=O)[O-])c1] | Br | 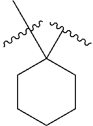 [SMILES: *C1(*)CCCCC1] | 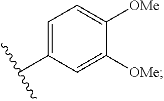 [SMILES: *c1ccc(OC)c(OC)c1] |
| 2009 | 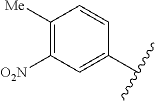 [SMILES: *c1ccc(C)c([N+](=O)[O-])c1] | NH₂ | 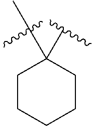 [SMILES: *C1(*)CCCCC1] | 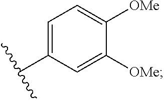 [SMILES: *c1ccc(OC)c(OC)c1] |
| 2010 | 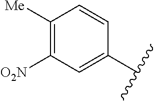 [SMILES: *c1ccc(C)c([N+](=O)[O-])c1] | NH₂ | 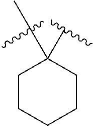 [SMILES: *C1(*)CCCCC1] | 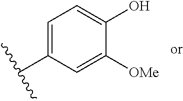 [SMILES: *c1ccc(O)c(OC)c1] |
| 2011 | 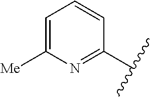 [SMILES: *c1cccc(C)n1] | NH₂ | 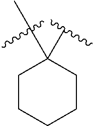 [SMILES: *C1(*)CCCCC1] | 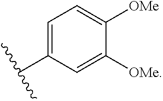 [SMILES: *c1ccc(OC)c(OC)c1] |

### Compounds of Formula (B13)

Compounds of the general Formula (B13) are described in PCT Publication No. WO 2006/136561, published Dec. 28, 2006, which is hereby incorporated by reference in its entirety. Formula (B13) has the structure:

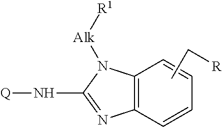

or a salt or a stereochemically isomeric form thereof, wherein: R can be a radical of formula

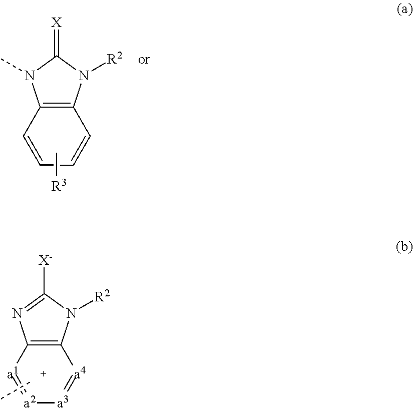

Q can be hydrogen or C₁₋₆alkyl optionally substituted with a heterocycle or Q is C₁₋₆alkyl substituted with both a radical —OR⁴ and a heterocycle; wherein said heterocycle is selected from oxazolidine, thiazolidine, 1-oxo-thiazolidine, 1,1-dioxothiazolidine, morpholinyl, thiomorpholinyl, 1-oxo-thiomorpholinyl, 1,1-dioxothiomorpholinyl, hexahydrooxazepine, hexahydrothiazepine, 1-oxo-hexahydrothiazepine, 1,1-dioxo-hexahydrothiazepine, pyrrolidine, piperidine, homopiperidine, piperazine; wherein each of said heterocycle may be optionally substituted with one or two substituents selected from the group consisting of C₁₋₆alkyl, hydroxyC₁₋₆alkyl, aminocarbonylC₁₋₆alkyl, hydroxy, carboxyl, C₁₋₆ alkyloxycarbonyl, aminocarbonyl, mono- or di(C₁₋₆alkyl)aminocarbonyl, C₁₋₆alkylcarbonylamino, aminosulfonyl and mono- or di(C₁₋₆alkyl)aminosulfonyl; AIk can be C₁₋₆ alkanediyl; X can be O or S; −a¹=a²−a³=a⁴− can be a bivalent radical of formula —N═CH—CH═CH—, —CH═N—CH═CH—, —CH═CH—N═CH— or —CH═CH—CH═N—; wherein one of the nitrogen atoms bears the chemical bond linking radical (b) with the rest of the molecule; R¹ can be Ar or a heterocycle selected from pyridyl, pyrazinyl, pyridazinyl, pyrimidinyl, furanyl, tetrahydrofuranyl, thienyl, pyrrolyl, thiazolyl, oxazolyl, imidazolyl, isothiazolyl, pyrazolyl, isoxazolyl, oxadiazolyl, quinolinyl, quinoxalinyl, benzofuranyl, benzothienyl, benzimidazolyl, benzoxazolyl, benzothiazolyl, pyridopyridyl, naphthiridinyl, 1H-imidazo[4,5-b]pyridinyl, 3H-imidazo[4,5-b]-pyridinyl, imidazo[1,2-a]pyridinyl and 2,3-dihydro-1,4-dioxino[2,3-b]pyridyl; wherein each of said heterocycle may optionally be substituted with 1, 2, or 3 substituents each independently selected from halo, hydroxy, amino, cyano, carboxyl, C₁₋₆alkyl, C₁₋₆alkyloxy, hydroxyC₁₋₆ alkyloxy, C₁₋₆ alkyl-oxy)C₁₋₆ alkyloxy, C₁₋₆ alkylthio, C₁₋₆ alkyloxyC₁₋₆ alkyl, hydroxyC₁₋₆alkyl, mono- or di(C₁₋₆ alkyl)amino, mono- or di(C₁₋₆ alkyl)aminoC₁₋₆ alkyl, polyhaloC₁₋₆ alkyl, C₁₋₆alkylcarbonylamino, C₁₋₆alkyloxycarbonyl, aminocarbonyl, mono- and di-C₁₋₆alkylaminocarbonyl; R² can be hydrogen, C₁₋₆alkyl, hydroxyC₁₋₆alkyl, C₁₋₆alkyloxyC₁₋₆alkyl, Ar—C₁₋₆alkyloxy-C₁₋₆alkyl, C₃₋₇ cycloalkyl, cyano-C₁₋₆alkyl, Ar—C₁₋₆alkyl, Het-C₁₋₆alkyl; R³ can be hydrogen, C₁₋₆alkyl, cyano, aminocarbonyl, polyhaloC₁₋₆alkyl, C₂₋₆alkenyl or C₂₋₆alkynyl; R⁴ can be hydrogen or C₁₋₆alkyl; each Ar independently can be phenyl or phenyl substituted with 1 to 5, such as 1, 2, 3 or 4, substituents selected from halo, hydroxy, amino, mono- or di(C₁₋₆ alkyl)amino, C₁₋₆ alkylcarbonylamino, C₁₋₆alkylsulfonylamino, cyano, C₁₋₆alkyl, C₂₋₆ alkenyl, C₂₋₆ alkynyl, phenyl, hydroxyC₁₋₆ alkyl, polyhaloC₁₋₆ alkyl, aminoC₁₋₆ alkyl, mono- or di(C₁₋₆ alkyl)aminoC₁₋₆ alkyl, C₁₋₆ alkyloxy, polyhaloC₁₋₆alkyloxy, phenoxy, aminocarbonyl, mono- or di(C₁₋₆alkyl)aminocarbonyl, hydroxycarbonyl, C₁₋₆alkoxycarbonyl, C₁₋₆alkylcarbonyl, aminosulfonyl, mono- and di(C₁₋₆alkyl)-aminosulfonyl; Het can be a heterocycle selected from pyridyl, pyrazinyl, pyridazinyl, pyrimidinyl, iuranyl, tetrahydrofuranyl, thienyl, pyrrolyl, thiazolyl, oxazolyl, imidazolyl, isothiazolyl, pyrazolyl, isoxazolyl, oxadiazolyl, quinolinyl, quinoxalinyl, benzofuranyl, benzothienyl, benzimidazolyl, benzoxazolyl, benzothiazolyl, pyridopyridyl, naphthiridinyl, 1H-imidazo[4,5-b]pyridinyl, 3H-imidazo[4,5-b]pyridinyl, imidazo[1,2-a]pyridinyl and 2,3-dihydro-1,4-dioxino-[2,3-b]pyridyl; wherein each Het may be optionally substituted with 1, 2 or 3 substituents each independently selected from halo, hydroxy, amino, mono- or di(C₁₋₆alkyl)amino, cyano, C₁₋₆alkyl, hydroxyC₁₋₆alkyl, polyhaloC₁₋₆alkyl, C₁₋₆alkyloxy.

Examples of Compounds of Formula (B13) include:

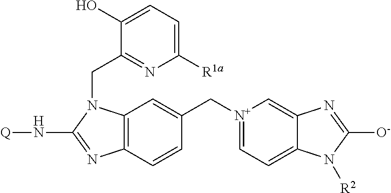

| Comp. | | | |
|---|---|---|---|
| Nr. | Q | R^{1a} | R² |
| a-6 | H- - - - | 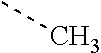 | 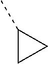 |
| c-4 | 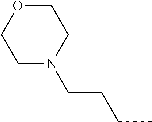 | 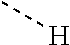 | 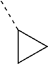 |
| b-4 | 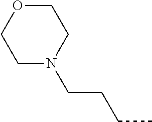 | 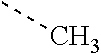 | 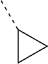 |
| d-5 | 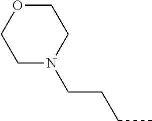 | 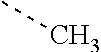 | 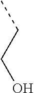 |
| e-7 | 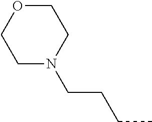 | 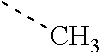 | 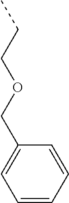 |
| m-9 | 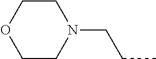 | 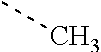 | 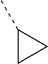 |
| n-7 | 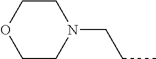 | 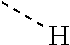 | 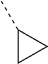 |
| i-3 | 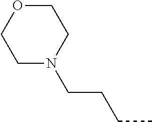 | 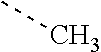 | 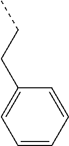 |
| h-4 | 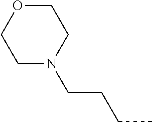 | 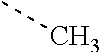 | 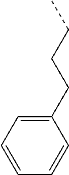 |
| o-3 | 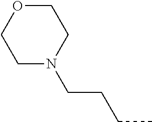 | 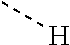 | 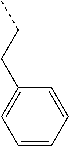 |
| p-3 | 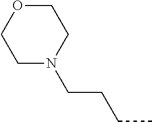 | 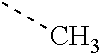 | 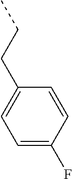 |
| g-7 | H- - - - | 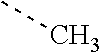 | 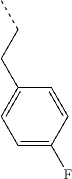 |
| q-3 | H- - - - | 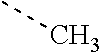 | 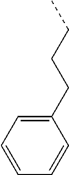 |
| j-4 | H- - - - | 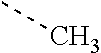 | 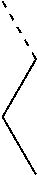 |

and

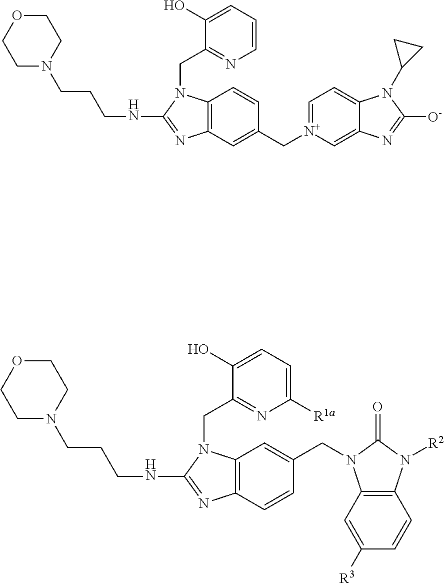

| Comp. | | | |
|---|---|---|---|
| Nr. | R^{1a} | R² | R³ |
| k-3 | 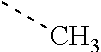 | 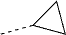 | 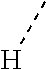 |
| l-3 | 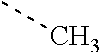 | 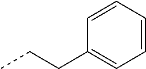 | 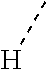 |
| l-4 | 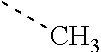 | 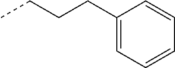 | 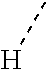 |
| f-6 | 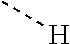 | 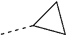 | 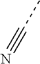 |

### Compounds of Formula (B14)

Compounds of the general Formula (B14) are described in PCT Publication No. WO 2005/058869, published Jun. 30, 2005, which is hereby incorporated by reference in its entirety. Formula (B14) has the structure:

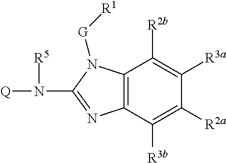

or a prodrug, N-oxide, addition salt, quaternary amine, metal complex, or a stereochemically isomeric form thereof, wherein: G can be a direct bond or C₁₋₁₀alkanediyl optionally substituted with one or more substituents independently selected from hydroxy, C₁₋₆alkyloxy, Ar¹ C₁₋₆alkyloxy, C₁₋₆alkylthio, Ar¹ C₁₋₆alkylthio, HO(—CH₂—CH₂—O)ₙ—, C₁₋₆alkyloxy(—CH₂—CH₂—O)ₐ— or Ar¹ C₁₋₆alkyloxy(—CH₂—CH₂—O)n-; each n independently can be 1, 2, 3 or 4; R¹ can be Ar¹ or a monocyclic or bicyclic heterocycle being selected from piperidinyl, piperazinyl, pyridyl, pyrazinyl, pyridazinyl, pyrimidinyl, furanyl, tetrahydro-furanyl, thienyl, pynolyl, thiazolyl, oxazolyl, imidazolyl, isothiazolyl, pyrazolyl, isoxazolyl, oxadiazolyl, quinolinyl, quinoxalinyl, benzofuranyl, benzothienyl, benzimidazolyl, benzoxazolyl, benzthiazolyl, pyridopyridyl, naphthiridinyl, 1H-imidazo[4,5-b]pyridinyl, 3H-imidazo[4,5-b]pyridinyl, imidazo[1,2-a]-pyridinyl, 2,3-dihydro-1,4-dioxino[2,3-b]pyridyl or a radical of formula

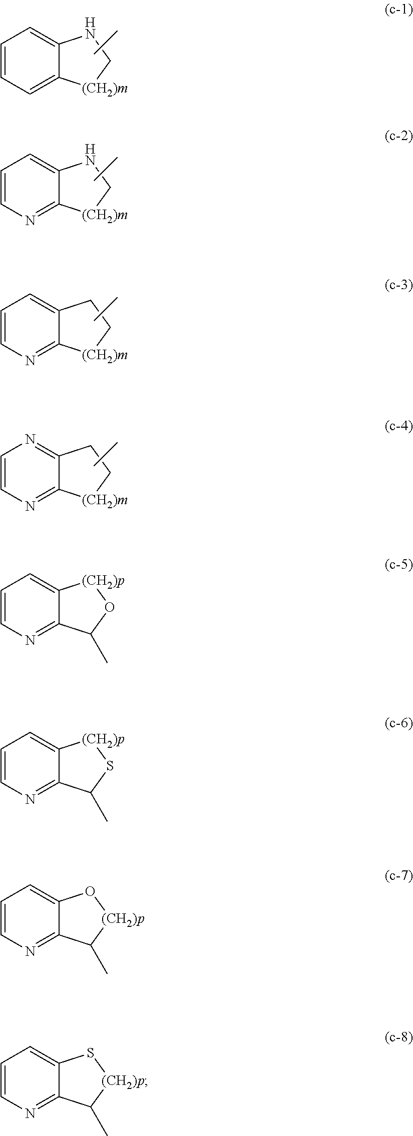

wherein each of said monocyclic or bicyclic heterocycles may optionally be substituted with 1 or where possible more, such as 2, 3, 4 or 5, substituents independently selected from halo, hydroxy, amino, cyano, carboxyl, C₁₋₆alkyl, C₁₋₆alkyloxy, C₁₋₆alkylthio, C₁₋₆alkyloxyCi-ealkyl, Ar¹, Ar¹C₁₋₆alkyl, Ar¹C₁₋₆alkyloxy, hydroxyC₁₋₆alkyl, mono- or di(C₁₋₆alkyl)amino, mono- or di(C₁₋₆alkyl)aminoC₁₋₆ alkyl, polyhaloC₁₋₆ alkyl, C₁₋₆ alkylcarbonylamino, C₁₋₆ alkyl-SO₂—NR^{5c}—, Ar¹—SO₂—NR^{5c}—, C₁₋₆alkyloxycarbonyl, —C(═O)—NR^{5c}R^{5d}, HO(—CH₂—CH₂—O)ₙ—, halo(—CH₂—CH₂—O)ₙ—, C₁₋₆alkyloxy(—CH₂—CH₂—O)ₙ—, Ar¹C₁₋₆alkyloxy(—CH₂—CH₂—O)ₙ— and mono- or di(C₁₋₆ alkyl)amino (—CH₂—CH₂—O)ₙ—; each m independently can be 1 or 2; each p independently can be 1 or 2; each t independently can be 0, 1 or 2; Q can be hydrogen, amino or mono- or di(C₁₋₄alkyl)amino; one of R^{2a} and R^{3a} can be selected from halo, optionally mono- or polysubstituted C₁₋₆alkyl, optionally mono- or polysubstituted C₂₋₆alkenyl, nitro, hydroxy, Ar², N(R^{4a}R⁴), N(R^{4a}R^{4b})sulfonyl, N(R^{4a}R⁴)carbonyl, C₁₋₆alkyloxy, Ar²oxy, Ar²C₁₋₆alkyloxy, carboxyl, C₁₋₆alkyloxycarbonyl, or —C(═Z)Ar²; and the other one of R^{2a} and R^{3a} is hydrogen; wherein ═Z is ═O, ═CH—C(═O)—NR^{5a}R^{5b}, ═CH₂, ═CH—C₁₋₆alkyl, ═N—OH or ═N—O—C₁₋₆alkyl; and the optional substituents on C₁₋₆alkyl and C₂₋₆ alkenyl can be the same or can be different relative to one another, and are each independently selected from hydroxy, cyano, halo, nitro, N(R^{4a}R^{4b}), N(R^{4a}R^{4b})sulfonyl, Het, Ar², C₁₋₆alkyloxy, C₁₋₆alkyl-S(═O)ₜ, Ar²oxy, Ar²—S(═O)ₜ, Ar²C₁₋₆alkyloxy, Ar²C₁₋₆alkyl-S(═O)ₜ, Het-oxy, Het-S(═O)ₜ, HetC₁₋₆alkyloxy, HetC₁₋₆alkyl-S(═O)ₜ, carboxyl, C₁₋₆alkyloxycarbonyl and —C(═Z)Ar²; in case R^{2a} is different from hydrogen then R^{2b} is hydrogen, C₁₋₆alkyl or halogen and R^{3b} is hydrogen; in case R^{1a} is different from hydrogen then R^{3b} is hydrogen, C₁₋₆alkyl or halogen and R^{2b} is hydrogen; R^{4a} and R^{4b} can be the same or can be different relative to one another, and can be each independently selected from hydrogen, C₁₋₆alkyl, Ar²C₁₋₆alkyl, (Ar²)(hydroxy) C₁₋₆alkyl, Het-C₁₋₆alkyl, hydroxyC₁₋₆alkyl, mono- and di-(C₁₋₆alkyloxy)C₁₋₆alkyl, (hydroxyC₁₋₆alkyl)oxyC₁₋₆alkyl, Ar¹C₁₋₆alkyloxy-C₁₋₆alkyl, dihydroxyC₁₋₆alkyl, C₁₋₆alkyloxy)(hydroxy)C₁₋₆alkyl, (Ar¹C₁₋₆alkyloxy)(hydroxy) C₁₋₆alkyl, Ar¹oxy-C₁₋₆alkyl, (Ar¹oxy)(hydroxy)-C₁₋₆alkyl, aminoC₁₋₆alkyl, mono- and di(C₁₋₆alkyl)amino-C₁₋₆alkyl, carboxyl-C₁₋₆alkyl, C₁₋₆alkyloxycarbonylC₁₋₆alkyl, aminocarbonylC₁₋₆alkyl, mono- and di(C₁₋₆alkyl)aminocarbonylC₁₋₆alkyl, C₁₋₆alkylcarbonylC₁₋₆alkyl, C₁₋₄alkyloxy)₂-P(═O)—C₁₋₆alkyl, C₁₋₄alkyloxy)₂P(═O)—O—C₁₋₆alkyl, aminosulfonyl-C₁₋₆alkyl, mono- and di(C₁₋₆alkyl)aminosulfonyl-C₁₋₆alkyl, C₁₋₆alkylcarbonyl, Ar²carbonyl, Het-carbonyl, Ar²C₁₋₆alkylcarbonyl, Het-C₁₋₆alkylcarbonyl, C₁₋₆alkylsulfonyl, aminosulfonyl, mono- and di(C₁₋₆alkyl)aminosulfonyl, Ar²sulfonyl, Ar²C₁₋₆alkylsulfonyl, Ar², Het, Het-sulfonyl, HetC₁₋₆alkylsulfonyl; R^{5a} and R^{5b} can be the same or can be different relative to one another, and are each independently hydrogen or C₁₋₆alkyl; or R^{5a} and R^{5b} taken together may form a bivalent radical of formula —(CH₂)ₛ— wherein s is 4 or 5; R^{5a} and R^{5d} can be the same or can be different relative to one another, and are each independently hydrogen or C₁₋₆alkyl; or R^{5c} and R^{5d} taken together may form a bivalent radical of formula —(CH₂)ₛ— wherein s is 4 or 5; R^{h}a can be hydrogen, C₁₋₆alkyl, Ar¹, Ar¹C₁₋₆alkyl, C₁₋₆alkylcarbonyl, Ar¹carbonyl, Ar¹C₁₋₆alkylcarbonyl, C₁₋₆alkylsulfonyl, Ar¹ sulfonyl, Ar¹C₁₋₆alkylsulfonyl, C₁₋₆alkyloxyC₁₋₆alkyl, aminoC₁₋₆alkyl, mono- or di(C₁₋₆alkyl)aminoC₁₋₆alkyl, hydroxyC₁₋₆alkyl, (carboxyl)-C₁₋₆alkyl, C₁₋₆alkyloxycarbonyl)-C₁₋₆alkyl, aminocarbonylC₁₋₆alkyl, mono- and di(C₁₋₆alkyl)aminocarbonylC₁₋₆alkyl, aminosulfonyl-C₁₋₆alkyl, mono- and di(C₁₋₆alkyl)aminosulfonyl-C₁₋₆alkyl, Het, Het-C₁₋₆alkyl, Het-carbonyl, Het-sulfonyl, Het-C₁₋₆alkylcarbonyl; R^{6b} can be hydrogen, C₁₋₆alkyl, Ar¹ or Ar¹C₁₋₆alkyl; R^{6c} can be C₁₋₆alkyl, Ar¹ or Ar¹C₁₋₆alkyl; Ar¹ can be phenyl or phenyl substituted with 1 or more, such as 2, 3 or 4, substituents selected from halo, hydroxy, C₁₋₆alkyl, hydroxyC₁₋₆alkyl, polyhaloC₁₋₆alkyl, and C₁₋₆alkyloxy; Ar² can be phenyl, phenyl annelated with C₅₋₇ cycloalkyl, or phenyl substituted with 1 or more, such as 2, 3, 4 or 5, substituents selected from halo, cyano, C₁₋₆alkyl, Het-C₁₋₆alkyl, Ar¹C₁₋₆alkyl, cyanoC₁₋₆alkyl, C₂₋₆alkenyl, cyanoC₂₋₆alkenyl, R^{6b}—O—C₃₋₆alkenyl, C₂₋₆alkynyl, cyanoC₂₋₆alkynyl, R^{6b}—O—C₃₋₆alkynyl, Ar¹, Het, R^{6b}—O—, R^{6b}—S—, R^{6c}—SO—, R^{6c}—SO₂—, R^{6b}—O—C₁₋₆alkyl-SO₂—, —N(R^{6a}R^{6b}), polyhalo-C₁₋₆alkyl, polyhaloC₁₋₆alkyloxy, polyhaloC₁₋₆alkylthio, R^{6c}—C(═O)—, R^{6b}—O—C(═O)—, —N(R^{6a}R^{6b})—C(═O)—, R^{6b}—O—C₁₋₁₀alkyl, R^{6b}—S—C₁₋₆alkyl, R^{6c}—S(═O)₂—C₁₋₆alkyl, —N(R^{6a}R^{6b})—C₁₋₆alkyl, R^{6c}—C(═O)—C₁₋₆alkyl, R^{6b}—O—C(═O)—C₁₋₆alkyl, N(R^{6a}R⁶)—C(═O)—C₁₋₆alkyl, R^{6c}—C(═O)—NR⁶—, R^{6c}—C(═O)—O—, R^{6c}—C(═O)—NR^{6b}C₁₋₆alkyl, R^{6c}—C(═O)—O—C₁₋₆alkyl, N(R^{6a}R^{6b})—S(═O)₂—, H₂N—C(═NH)—; Het can be a heterocycle being selected from tetrahydrofuranyl, tetrahydrothienyl, pynolidinyl, pynolidinonyl, furanyl, thienyl, pynolyl, thiazolyl, oxazolyl, imidazolyl, isothiazolyl, pyrazolyl, isoxazolyl, oxadiazolyl, thiadiazolyl, piperidinyl, homopiperidinyl, piperazinyl, moφholinyl, pyridyl, pyrazinyl, pyridazinyl, pyrirmdinyl, tetrahydroquinolinyl, quinolinyl, isoquinolinyl, benzodioxanyl, benzodioxolyl, indolinyl, indolyl, each of said heterocycle may optionally be substituted with oxo, amino, Ar¹, C₁₋₄alkyl, aminoC₁₋₄alkyl, Ar¹C₁₋₄alkyl, mono- or di(C₁₋₆alkyl)aminoC₁₋₆ alkyl, mono- or di(C₁₋₆ alkyl)amino, (hydroxyC₁₋₆ alkyl)amino, and optionally further with one or two C₁₋₄alkyl radicals.

Examples of Compounds of Formula (B14) include:

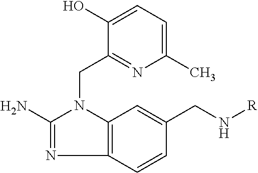

| Comp. | |
|---|---|
| No. | R |
| 1 | 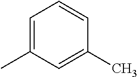 |
| 2 | 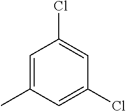 |
| 3 | 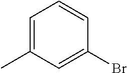 |
| 4 | 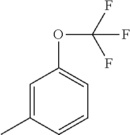 |
| 5 | 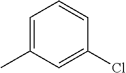 |
| 6 | 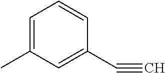 |
| 7 | 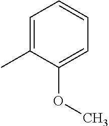 |
| 8 | 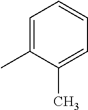 |
| 9 | 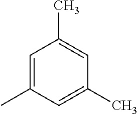 |
| 10 | 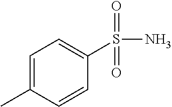 |
| 11 | 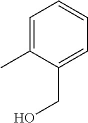 |
| 12 | 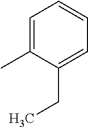 |
| 13 | 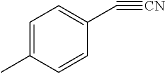 |
| 14 | 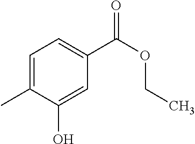 |
| 15 | 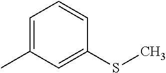 |
| 16 | 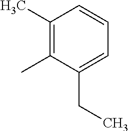 |
| 17 | 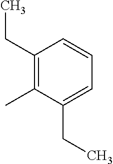 |
| 18 | 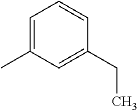 |
| 19 | 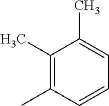 |
| 20 | 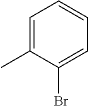 |
| 21 | 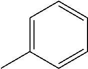 |
| 22 | 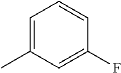 |
| 23 | 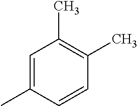 |
| 24 | 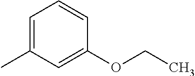 |
| 25 | 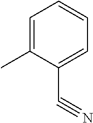 |
| 26 | 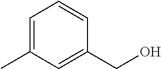 |
| 27 | 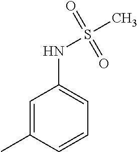 |
| 28 | 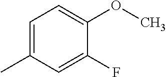 |
| 29 | 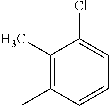 |
| 30 | 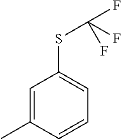 |
| 31 | 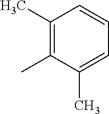 |
| 32 | 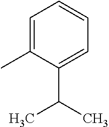 |
| 33 | 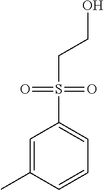 |
| 34 | 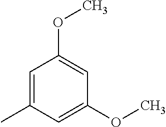 |
| 35 | 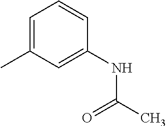 |
| 36 | 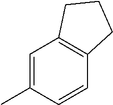 |
| 37 | 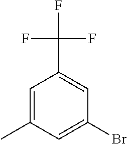 |
| 38 | 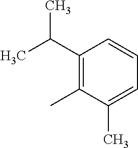 |
| 39 | 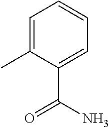 |
| 40 | 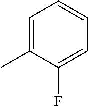 |
| 41 | 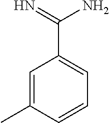 |
| 42 | 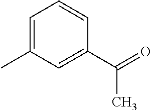 |
| 43 | 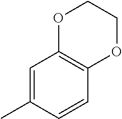 |
| 44 | 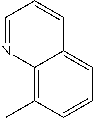 |
| 45 | 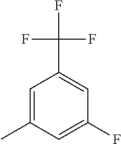 |
| 46 | 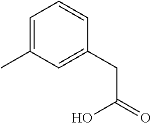 |
| 47 | 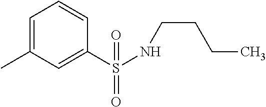 |
| 48 | 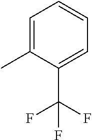 |
| 49 | 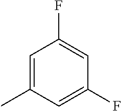 |
| 50 | 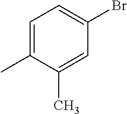 |
| 51 | 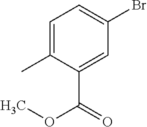 |
| 52 | 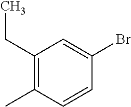 |
| 53 | 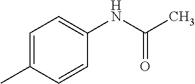 |
| 54 | 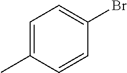 |
| 55 | 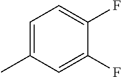 |
| 56 | 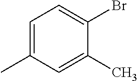 |
| 57 | 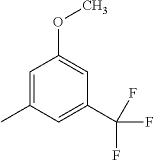 |
| 58 | 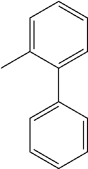 |
| 59 | 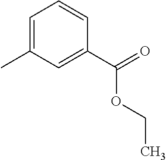 |
| 60 | 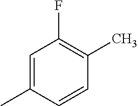 |
| 61 | 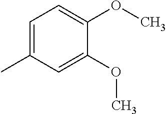 |
| 62 | 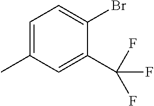 |
| 63 | 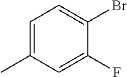 |
| 64 | 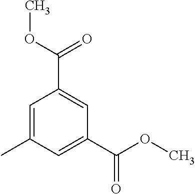 |
| 65 | 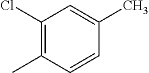 |
| 66 | 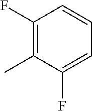 |
| 67 | 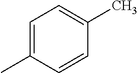 |
| 68 | 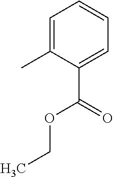 |
| 69 | 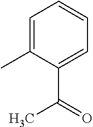 |
| 70 | 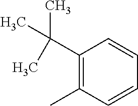 |
| 71 | 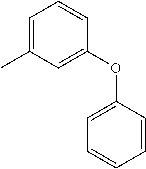 |
| 72 | 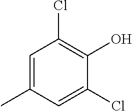 |
| 73 | 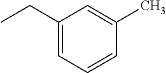 |
| 74 | 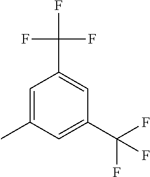 |
| 75 | 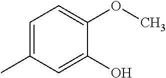 |
| 76 | 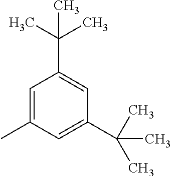 |
| 77 | 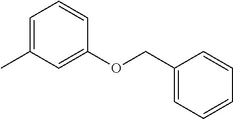 |
| 78 | 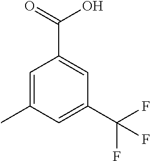 |
| 79 | 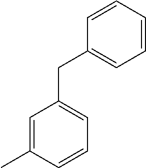 |
| 80 | 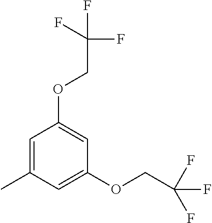 |
| 81 | 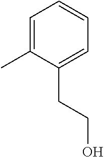 |
| 82 | 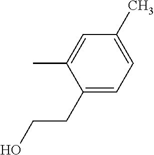 |
| 83 | 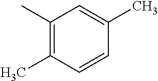 |
| 84 | 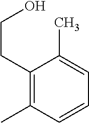 |
| 85 | 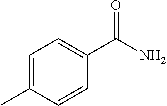 |
| 86 | 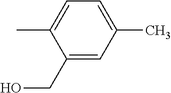 |
| 87 | 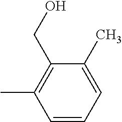 |
| 88 | 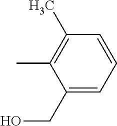 |
| 89 | 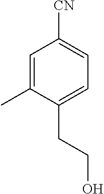 |
| 90 | 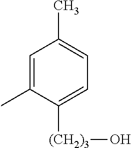 |
| 91 | 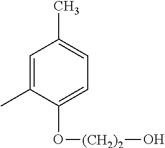 |
| 92 | 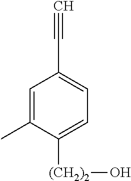 |
| 93 | 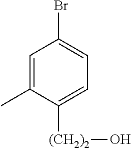 |
| 94 | 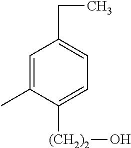 |
| 95 | 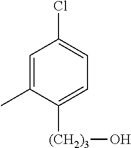 |
| 96 | 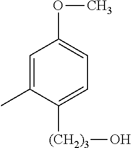 |
| 97 | 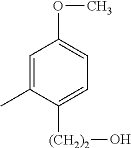 |
| 98 | 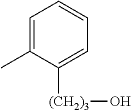 |
| 99 | 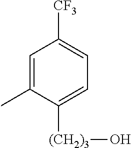 |
| 100 | 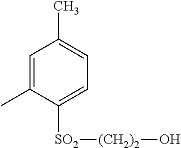 |
| 101 | 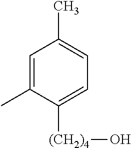 |
| 102 | 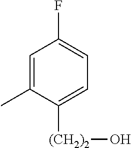 |
| 103 | 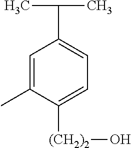 |
| 104 | 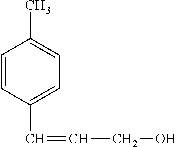 |
| 105 | 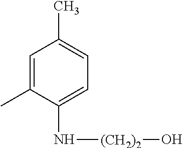 |
| 106 | 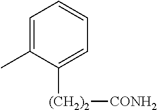 |
| 107 | 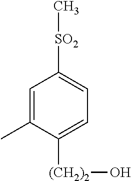 |
| 108 | 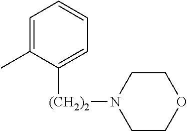 |
| 109 | 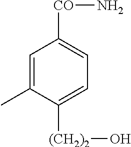 |
| 110 | 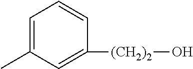 |
| 111 | 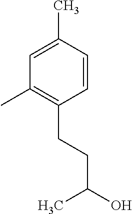 |
| 112 | 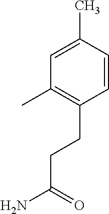 |
| 113 | 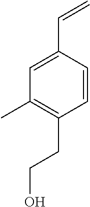 |
| 114 | 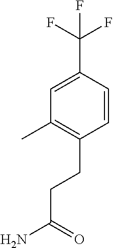 |
| 115 | 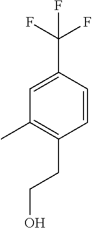 |

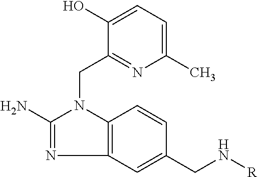

| Comp. | |
|---|---|
| No. | R |
| 116 | 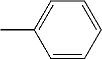 |
| 117 | 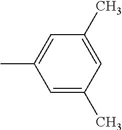 |
| 118 | 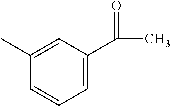 |
| 119 | 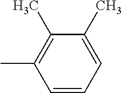 |
| 120 | 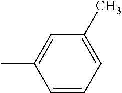 |
| 121 | 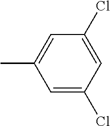 |
| 122 | 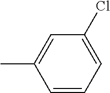 |
| 123 | 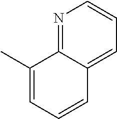 |
| 124 | 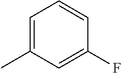 |
| 125 | 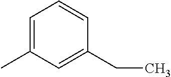 |
| 126 | 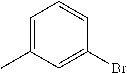 |
| 127 | 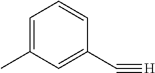 |

and

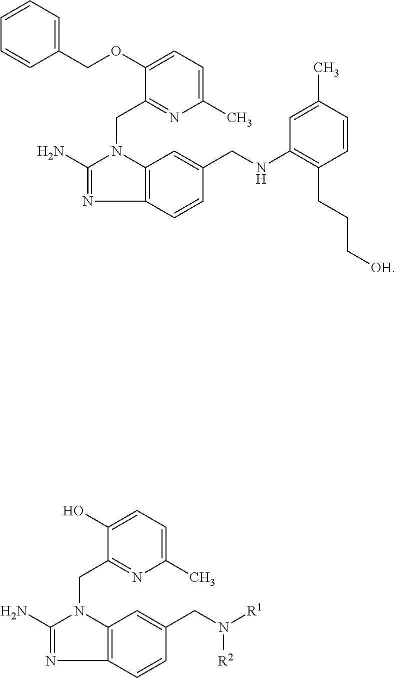

| Comp. | | |
|---|---|---|
| No. | R¹ | R² |
| 129 | 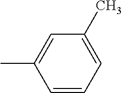 | 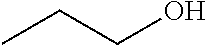 |
| 130 | 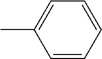 | 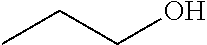 |
| 131 | 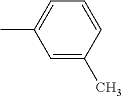 | 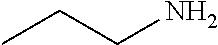 |
| 132 | 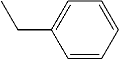 | 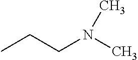 |
| 133 | 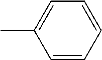 | 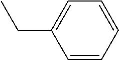 |
| 134 | 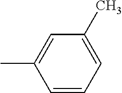 | 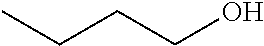 |
| 135 | 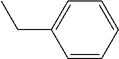 | 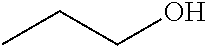 |
| 136 | 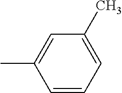 | 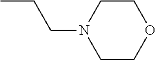 |
| 137 | 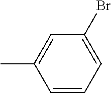 | 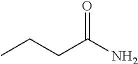 |
| 138 | 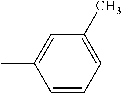 | 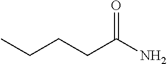 |
| 139 | 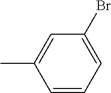 | 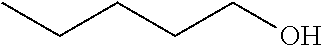 |
| 140 | 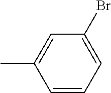 | 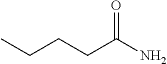 |
| 141 | 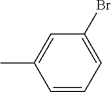 | 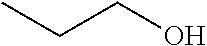 |
| 142 | 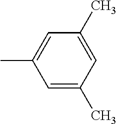 | 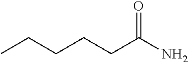 |
| 143 | 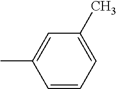 | 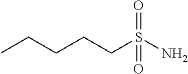 |
| 144 | 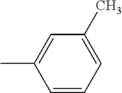 | 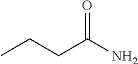 |
| 145 | 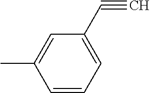 | 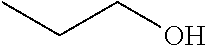 |
| 146 | 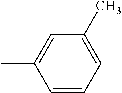 | 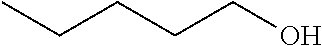 |
| 147 | 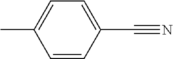 | 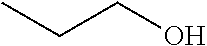 |
| 148 | 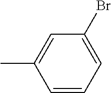 | 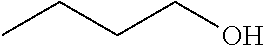 |
| 149 | 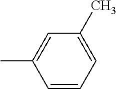 | 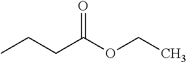 |
| 150 | 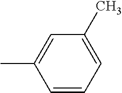 | 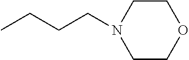 |
| 151 | 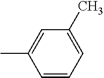 | 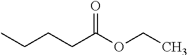 |
| 152 | 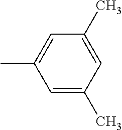 | 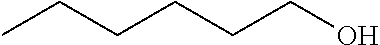 |
| 153 | 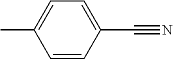 | 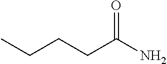 |
| 154 | 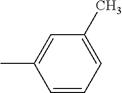 | 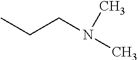 |
| 155 | 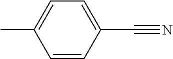 | 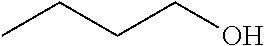 |
| 156 | 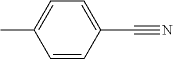 | 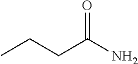 |
| 157 | 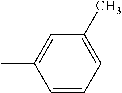 | 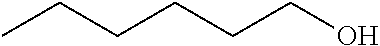 |
| 158 | 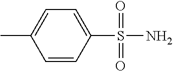 | 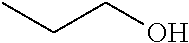 |
| 159 | 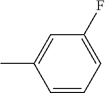 | 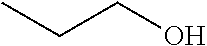 |
| 160 | 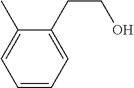 | 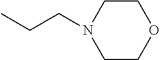 |
| 161 | 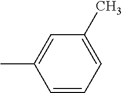 | 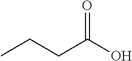 |
| 162 | 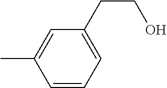 | 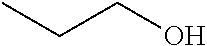 |
| 163 | 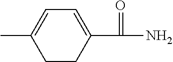 | 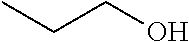 |
| 164 | 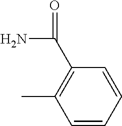 | 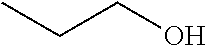 |
| 165 | 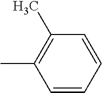 | 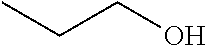 |
| 166 | 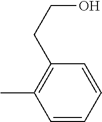 | —CH₃ |
| 167 | 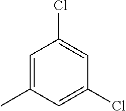 | —(CH₂)₂—CO—NH₂ |
| 168 | 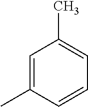 | —(CH₂)₃—SO₂—NH₂ |
| 169 | 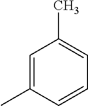 | 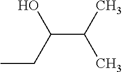 |
| 170 | 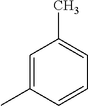 | 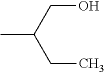 |
| 171 | 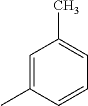 | 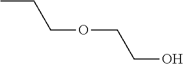 |
| 172 | 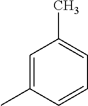 | 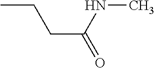 |
| 173 | 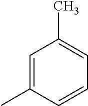 | 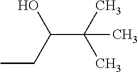 |
| 174 | 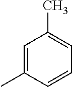 | 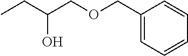 |
| 175 | 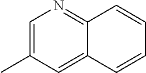 | 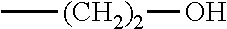 |
| 176 | 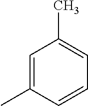 | 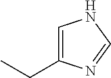 |
| 177 | 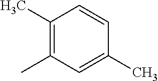 | 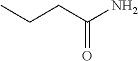 |
| 178 | 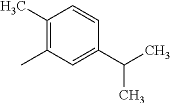 | 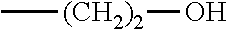 |
| 179 | 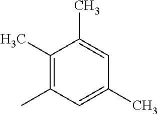 | 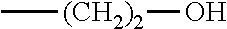 |
| 180 | 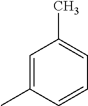 | 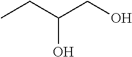 |
| 181 | 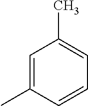 | 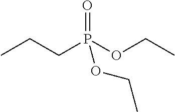 |
| 182 | 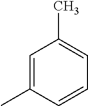 | 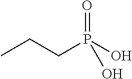 |

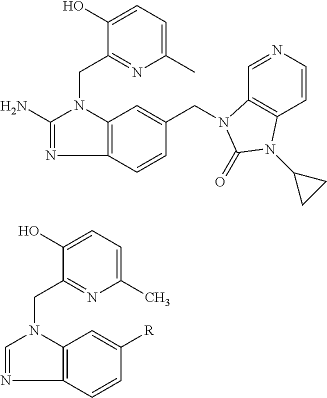

and

| Comp. | |
|---|---|
| No. | R |
| 184 | 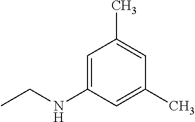 |
| 185 | 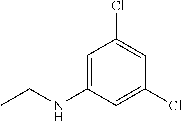 |
| 186 | 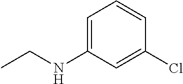 |
| 187 | 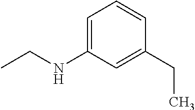 |
| 188 | 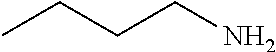 |
| 189 | 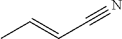 |
| 190 | 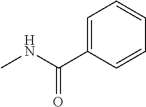 |
| 191 | 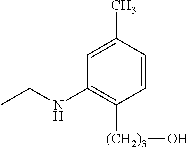 |
| 192 | 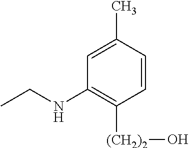 |
| 193 | 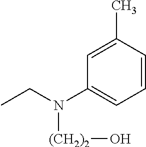 |

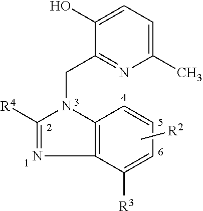

| Comp. | | | |
|---|---|---|---|
| No. | R² | R³ | R⁴ |
| 194 | 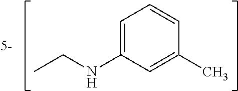 | —CH₃ | —NH₂ |
| 195 | 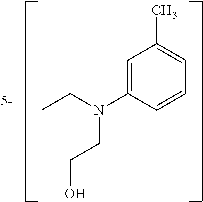 | H | —NH—CH₃ |
| 196 | 5-[—(CH₂)₃—NH₂] | H | —NH₂ |
| 197 | 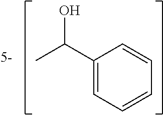 | H | —NH₂ |
| 198 | 5- [—(CH₂)₂—CN] | H | —NH₂ |
| 199 | 5- [—CH₃] | —CH₃ | —NH₂ |
| 200 | 5- [—CH₂—OH] | H | —NH—CH₃ |
| 201 | 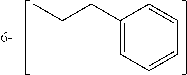 | H | —NH₂ |
| 202 | 6- [—CH₂—OH] | H | —NH—CH₃ |
| 203 | 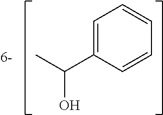 | H | —NH₂ |
| 204 | 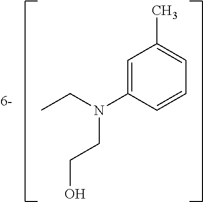 | H | —NH—CH₃ |

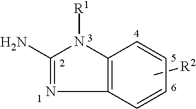

| Comp. | | |
|---|---|---|
| No. | R¹ | R² |
| 205 | 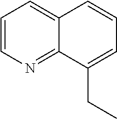 | 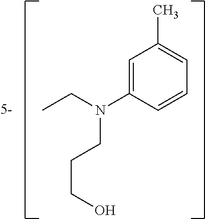 |
| 206 | 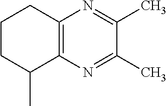 | 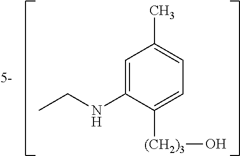 |
| 207 | 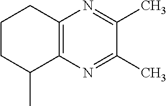 | 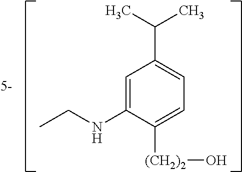 |
| 208 | 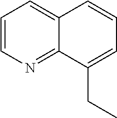 | 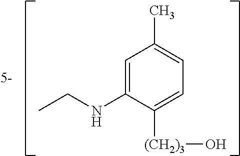 |
| 209 | 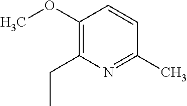 | 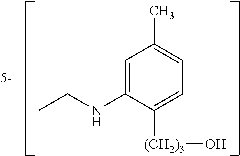 |
| 210 | 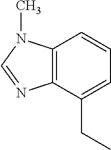 | 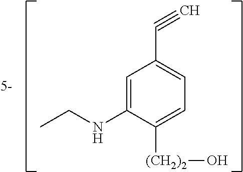 |
| 211 | 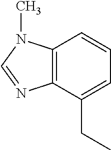 | 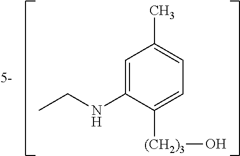 |
| 212 | 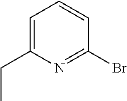 | 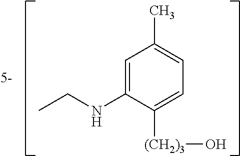 |
| 213 | 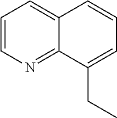 | 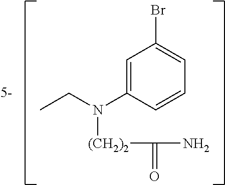 |
| 214 | 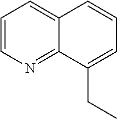 | 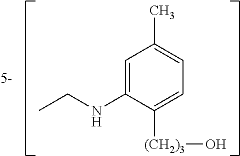 |
| 215 | 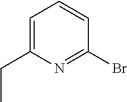 | 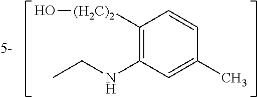 |
| 216 | 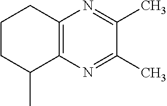 | 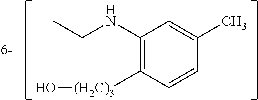 |
| 217 | 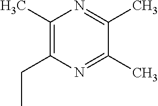 | 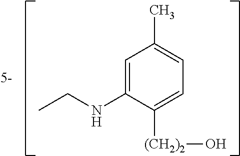 |
| 218 | 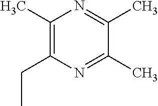 | 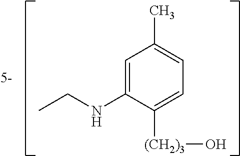 |
| 219 | 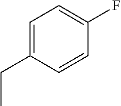 | 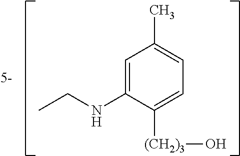 |
| 220 | 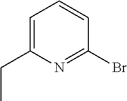 | 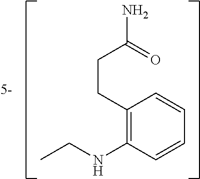 |
| 221 | 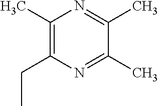 | 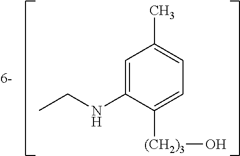 |
| 222 | 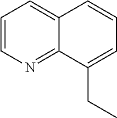 | 5-(CH₂—OH) |
| 223 | 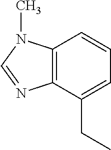 | 5-(CH₂—OH) |
| 224 | 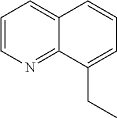 | 6-(CH₂—OH) |
| 225 | 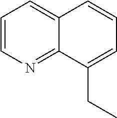 | 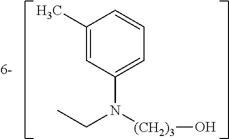 |
| 226 | 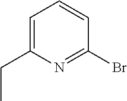 | 5-(CH₂—OH) |
| 227 | 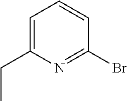 | 6-(CH₂—OH) |
| 228 | 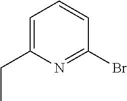 | 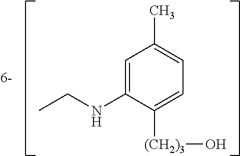 |
| 229 | 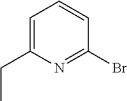 | 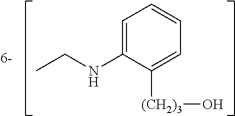 |
| 230 | 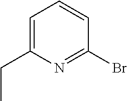 | 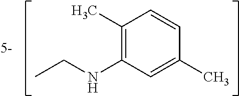 |
| 231 | 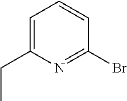 | 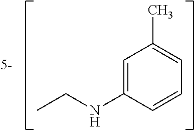 |
| 232 | 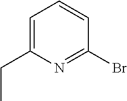 | 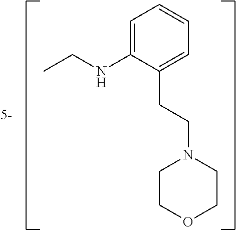 |
| 233 | 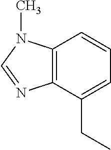 | 6-(CH₂—OH) |
| 234 | 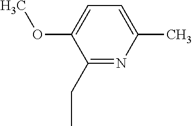 | 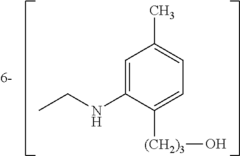 |
| 235 | 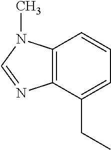 | 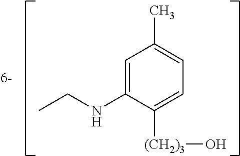 |
| 236 | 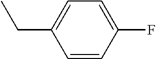 | 5-(CH₂—OH) |
| 237 | 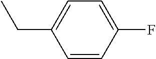 | 6-(CH₂—OH) |
| 238 | 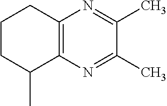 | 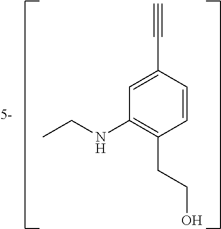 |
| 239 | 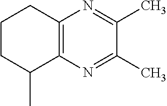 | 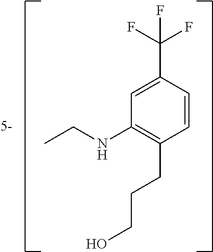 |
| 240 | 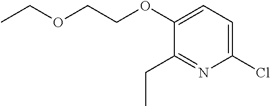 | 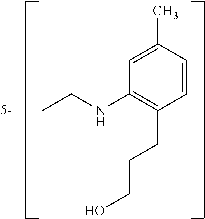 |

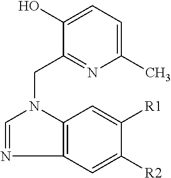

| Comp. | | |
|---|---|---|
| No. | R1 | R2 |
| 241 | H | 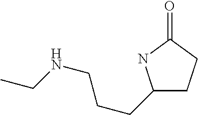 |
| 242 | H | 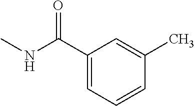 |
| 243 | H | 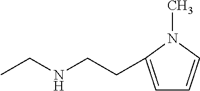 |
| 244 | H | 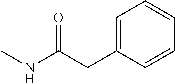 |
| 245 | H | 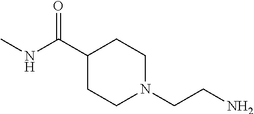 |
| 246 | H | —CH₂—OH |
| 247 | —CH₂—OH | H |
| 248 | 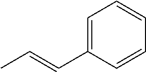 | H |
| 249 | H | 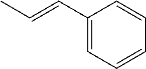 |
| 250 | H | 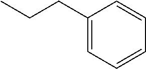 |
| 251 | 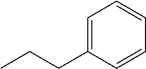 | H |
| 252 | H | —CH═CH—CN |
| 253 | H | 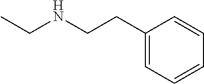 |
| 254 | 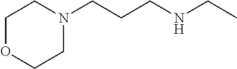 | H |
| 255 | H | 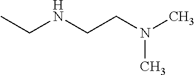 |
| 256 | H | 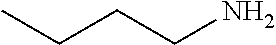 |
| 257 | 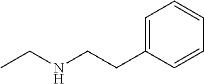 | H |
| 258 | H | 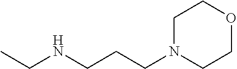 |
| 259 | H | —NH₂ |
| 260 | H | 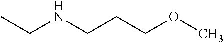 |
| 261 | —NH₂ | H |
| 262 | H | 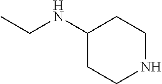 |
| 263 | 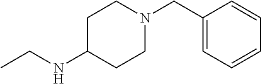 | H |
| 264 | 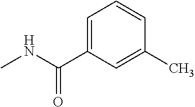 | H |
| 265 | H | 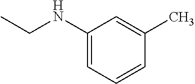 |
| 266 | H | 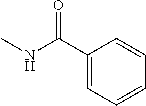 |
| 267 | H | 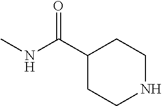 |
| 268 | 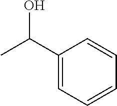 | H |
| 269 | H | 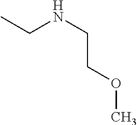 |
| 270 | H | 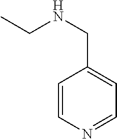 |
| 271 | H | 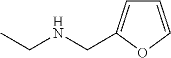 |
| 272 | H | 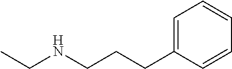 |
| 273 | H | 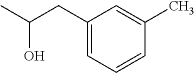 |
| 274 | H | 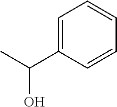 |
| 275 | 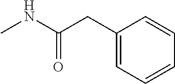 | H |
| 276 | 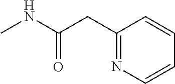 | H |
| 277 | 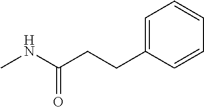 | H |
| 278 | 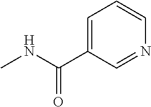 | H |
| 279 | H | 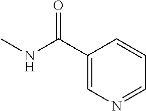 |
| 280 | H | 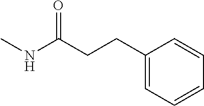 |
| 281 | H | 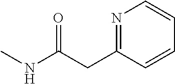 |
| 282 | 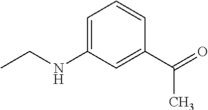 | H |
| 283 | 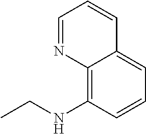 | H |
| 284 | 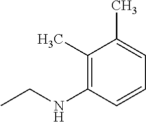 | H |
| 285 | 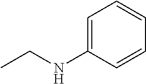 | H |
| 286 | H | 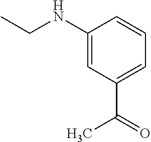 |
| 287 | H | 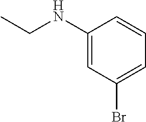 |
| 288 | H | 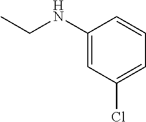 |
| 289 | H | 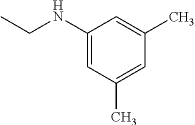 |
| 290 | H | 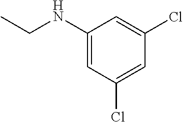 |
| 291 | H | 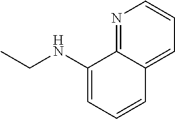 |
| 292 | H | 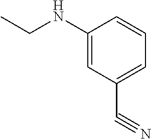 |
| 293 | H | 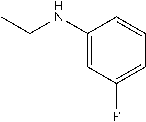 |
| 294 | H | 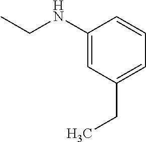 |
| 295 | H | 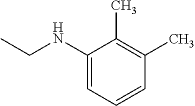 |
| 296 | H | 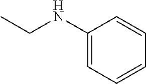 |

### Compounds of Formula (B15)

Compounds of the general Formula (B15) are described in U.S. Publication No. 2013/0090328, published Apr. 11, 2013, which is hereby incorporated by reference in its entirety. Formula (B15) has the structure:

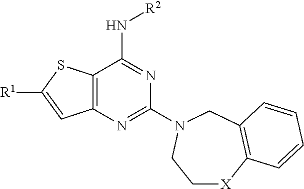

or a pharmaceutically acceptable salt or stereoisomer thereof, wherein: R¹ can be hydrogen or a C₁₋₆alkyl; R² can be (1) amino(CH₂)₂₋₆; (2) amino(CH₂)₁₋₆difluoromethyl(CH₂)₁₋₆; (3) amino(CH₂)₁₋₆fluoromethyl(CH₂)₁₋₆; (4) amino(CH₂)₀₋₆oxetanyl(CH₂)₁₋₆; (5) amino(CH₂)₁₋₆oxetanyl(CH₂)₀₋₆; or (6) pyrrolidin-3-yl, unsubstituted or 4-substituted by halogen; and X can be —O—, —S—, —S(═O)—, —S(O₂)—, —CH₂—, —CF₂— or —NH—.

Examples of Compounds of Formula (B15) include: N-[2-(1,1-dioxido-2,3-dihydro-1,4-benzothiazepin-4(5H)-yl)thieno[3,2-d]pyrimidin-4-yl]-2,2-difluoropropane-1,3-diamine; N-[2-(1-oxido-2,3-dihydro-1,4-benzothiazepin-4(5H)-yl)thieno[3,2-d]pyrimidin-4-yl]propane-1,3-diamine; N-[2-(2,3-dihydro-1,4-benzothiazepin-4(5H)-yl)thieno[3,2-d]pyrimidin-4-yl]propane-1,3-diamine; N-[2-(2,3-dihydro-1,4-benzothiazepin-4(5H)-yl)-6-methylthieno[3,2-d]pyrimidin-4-yl]propane-1,3-diamine; N-[2-(1,1-dioxido-2,3-dihydro-1,4-benzothiazepin-4(5H)-yl)-6-methylthieno[3,2-d]pyrimidin-4-yl]propane-1,3-diamine; N-[2-(1,1-dioxido-2,3-dihydro-1,4-benzothiazepin-4(5H)-yl)thieno[3,2-d]pyrimidin-4-yl]propane-1,3-diamine; N-[(3-aminooxetan-3-yl)methyl]-2-(1,1-dioxido-2,3-dihydro-1,4-benzothiazepin-4(5H)-yl)thieno[3,2-d]pyrimidin-4-amine; N-[3-(aminomethyl)oxetan-3-yl]-2-(1,1-dioxido-2,3-dihydro-1,4-benzothiazepin-4(5H)-yl)thieno[3,2-d]pyrimidin-4-amine; N-[(3-aminooxetan-3-yl)methyl]-2-[(1R)-1-oxido-2,3-dihydro-1,4-benzothiazepin-4(5H)-yl]thieno[3,2-d]pyrimidin-4-amine; N-[(3-aminooxetan-3-yl)methyl]-2-[(1S)-1-oxido-2,3-dihydro-1,4-benzothiazepin-4(5H)-yl]thieno[3,2-d]pyrimidin-4-amine; N-[(3-aminooxetan-3-yl)methyl]-6-methyl-2-(1-oxido-2,3-dihydro-1,4-benzothiazepin-4(5H)-yl)thieno[3,2-d]pyrimidin-4-amine; 2-fluoro-N-[6-methyl-2-(1-oxido-2,3-dihydro-1,4-benzothiazepin-4(5H)-yl)thieno[3,2-d]pyrimidin-4-yl]propane-1,3-diamine; N-[6-methyl-2-(1-oxido-2,3-dihydro-1,4-benzothiazepin-4(5H)-yl)thieno[3,2-d]pyrimidin-4-yl]ethane-1,2-diamine; N-{[3-(aminomethyl)oxetan-3-yl]methyl}-6-methyl-2-(1-oxido-2,3-dihydro-1,4-benzothiazepin-4(5H)-yl)thieno[3,2-d]pyrimidin-4-amine; N-[6-methyl-2-(1-oxido-2,3-dihydro-1,4-benzothiazepin-4(5H)-yl)thieno[3,2-d]pyrimidin-4-yl]propane-1,3-diamine; N-[trans-(±)-4-fluoropyrrolidin-3-yl]-6-methyl-2-(1-oxido-2,3-dihydro-1,4-benzothiazepin-4(5H)-yl)thieno[3,2-d]pyrimidin-4-amine; 6-methyl-2-(1-oxido-2,3-dihydro-1,4-benzothiazepin-4(5H)-yl)-N-(pyrrolidin-3-yl)thieno[3,2-d]pyrimidin-4-amine; N-[6-methyl-2-(1,2,3,5-tetrahydro-4H-1,4-benzodiazepin-4-yl)thieno[3,2-d]pyrimidin-4-yl]propane-1,3-diamine; N-[2-(1,2,3,5-tetrahydro-4H-1,4-benzodiazepin-4-yl)thieno[3,2-d]pyrimidin-4-yl]propane-1,3-diamine; N-[2-(2,3-dihydro-1,4-benzoxazepin-4(5H)-yl)thieno[3,2-d]pyrimidin-4-yl]propane-1,3-diamine; N-[2-(5,5-difluoro-1,3,4,5-tetrahydro-2H-2-benzazepin-2-yl)thieno[3,2-d]pyrimidin-4-yl]propane-1,3-diamine; N-{[3-(aminomethyl)oxetan-3-yl]methyl}-2-(5,5-difluoro-1,3,4,5-tetrahydro-2H-2-benzazepin-2-yl)thieno[3,2-d]pyrimidin-4-amine; N-[2-(1,3,4,5-tetrahydro-2H-2-benzazepin-2-yl)thieno[3,2-d]pyrimidin-4-yl]propane-1,3-diamine; N-[2-(2,3-dihydro-1,4-benzoxazepin-4(5H)-yl)-6-methylthieno[3,2-d]pyrimidin-4-yl]propane-1,3-diamine; N-[2-(5,5-difluoro-1,3,4,5-tetrahydro-2H-2-benzazepin-2-yl)-6-methylthieno[3,2-d]pyrimidin-4-yl]propane-1,3-diamine; and N-[6-methyl-2-(1,3,4,5-tetrahydro-2H-2-benzazepin-2-yl)thieno[3,2-d]pyrimidin-4-yl]propane-1,3-diamine.

### Compounds of Formula (B16)

Compounds of the general Formula (B16) are described in PCT Publication No. WO 2014/009302, published Jan. 16, 2014, which is hereby incorporated by reference in its entirety. Formula (B16) has the structure:

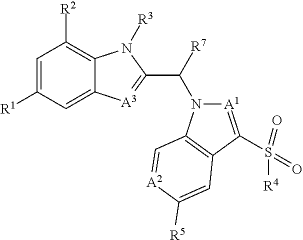

or pharmaceutically acceptable salts thereof, wherein: R¹ can be hydrogen or halogen; R² can be hydrogen or halogen; R³ can be azetidinyl; C₁₋₆alkoxypyridinyl; C₁₋₆alkylsulfonyl-CₓH₂ₓ—; carboxycycloalkyl; difluorocycloalkyl; 1,1-dioxo-tetrahydrothienyl; halopyridinyl; hydroxy-C_{y}H_{2y}—; hydroxy-CₓH₂ₓ-cycloalkyl; hydroxy-C_{y}H_{2y}—O—C_{y}H_{2y}—; hydroxycycloalkyl-C_{z}H_{2z}—, unsubstituted or substituted by C₁₋₃alkyl, hydroxy or hydroxy-CₓH₂ₓ—; 4-hydroxypiperidin-1-yl-C_{y}H_{2y}—; 3-hydroxy-pyrrolidin-1-yl-C_{y}H_{2y}—; morpholinyl-C_{y}H_{2y}—; oxetanyl; oxetanyl-CₓH₂ₓ—, unsubstituted or substituted by C₁₋₃alkyl; piperidinyl; oxo-piperidinyl; oxo-pyrrolidinyl; pyrrolidinyl, unsubstituted or substituted by C₁₋₆alkylcarbonyl, C₁₋₆alkylsulfonyl, hydroxy-C_{y}H_{2y}—, hydroxy-CₓH₂ₓ-carbonyl, amino-CₓH₂ₓ-carbonyl or trifluoromethyl-CₓH₂ₓ—; tetrahydrofuran-3-yl-C_{z}H_{2z}—; tetrahydropyranyl; trifluoromethyl-CₓH₂ₓ—;

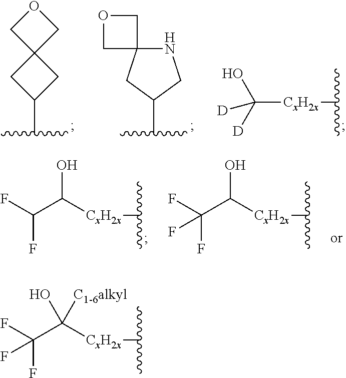

R⁴ can be C₁₋₆alkyl or cycloalkyl; R⁵ can be hydrogen or halogen; R⁷ can be hydrogen or C₁₋₆alkyl; A¹ can be —N— or —CH; A² can be —N—, —NO or —CH; A³ can be —N— or —CH; x can be 1-6; y can be 2-6; z can be 0-6.

Examples of Compounds of Formula (B16) include: 1-[2-(methylsulfonyl)ethyl]-2-{[3-(methylsulfonyl)-1H-indol-1-yl]methyl}-1H-benzimidazole; 5-chloro-2-{[3-(methylsulfonyl)-1H-indol-1-yl]methyl}-1-[3-(methylsulfonyl)propyl]-1H-benzimidazole; 5-chloro-2-{[5-fluoro-3-(methylsulfonyl)-1H-indol-1-yl]methyl}-1-[3-(methylsulfonyl)propyl]-1H-benzimidazole; 5-chloro-1-[3-(methylsulfonyl)propyl]-2-{[3-(methylsulfonyl)-1H-pyrrolo[2,3-c]pyridin-1-yl]methyl}-1H-benzimidazole; 5-chloro-2-{[3-(ethylsulfonyl)-1H-indol-1-yl]methyl}-1-[3-(methylsulfonyl)propyl]-1H-benzimidazole; 5-chloro-1-[3-(methylsulfonyl)propyl]-2-{[3-(propan-2-ylsulfonyl)-1H-indol-1-yl]methyl}-1H-benzimidazole; 5-chloro-2-{[3-(cyclopropylsulfonyl)-1H-indol-1-yl]methyl}-1-[3-(methylsulfonyl)propyl]-1H-benzimidazole; 1-({5-chloro-1-[3-(methylsulfonyl)propyl]-1H-benzimidazol-2-yl}methyl)-3-(methylsulfonyl)-1H-indazole; 1-({5-chloro-1-[3-(methylsulfonyl)propyl]-1H-benzimidazol-2-yl}methyl)-3-(propan-2-ylsulfonyl)-1H-indazole; 1-({5-chloro-1-[3-(methylsulfonyl)propyl]-1H-benzimidazol-2-yl}methyl)-3-(ethylsulfonyl)-1H-indazole; 1-({5-chloro-1-[3-(methylsulfonyl)propyl]-1H-benzimidazol-2-yl}methyl)-3-(methylsulfonyl)-1H-pyrazolo[3,4-c]pyridine; 1-({5-chloro-1-[2-(methylsulfonyl)ethyl]-1H-benzimidazol-2-yl}methyl)-3-(methylsulfonyl)-1H-indazole; 1-({5-chloro-1-[2-(methylsulfonyl)ethyl]-1H-benzimidazol-2-yl}methyl)-3-(propan-2-ylsulfonyl)-1H-indazole; 1-({5-chloro-1-[(3R)-1,1-dioxidotetrahydrothiophen-3-yl]-1H-benzimidazol-2-yl}methyl)-3-(methylsulfonyl)-1H-pyrazolo[3,4-c]pyridine; 1-{[5-chloro-1-(1,1-dioxidotetrahydrothiophen-3-yl)-1H-benzimidazol-2-yl]methyl}-3-(methylsulfonyl)-1H-indazole; 1-{[5-chloro-1-(oxetan-3-yl)-1H-benzimidazol-2-yl]methyl}-3-(methylsulfonyl)-1H-indazole; 4-(5-chloro-2-{[3-(methylsulfonyl)-1H-pyrazolo[3,4-c]pyridin-1-yl]methyl}-1H-benzimidazol-1-yl)piperidin-2-one; 1-{[5-chloro-1-(oxetan-3-yl)-1H-benzimidazol-2-yl]methyl}-3-(methylsulfonyl)-1H-pyrazolo[3,4-c]pyridine; 1-{[5-chloro-1-(tetrahydro-2H-pyran-4-yl)-1H-benzimidazol-2-yl]methyl}-3-(methylsulfonyl)-1H-indazole; 1-{[5-chloro-1-(tetrahydro-2H-pyran-4-yl)-1H-benzimidazol-2-yl]methyl}-3-(methylsulfonyl)-1H-pyrazolo[3,4-c]pyridine; 1-{[5-chloro-1-(tetrahydrofuran-3-yl)-1H-benzimidazol-2-yl]methyl}-3-(methylsulfonyl)-1H-indazole; 1-{[5-chloro-1-(3,3-difluorocyclopentyl)-1H-benzimidazol-2-yl]methyl}-3-(methylsulfonyl)-1H-indazole; 1-{[5-chloro-1-(3,3-difluorocyclopentyl)-1H-benzimidazol-2-yl]methyl}-3-(methylsulfonyl)-1H-pyrazolo[3,4-c]pyridine; 4-(5-chloro-2-{[3-(methylsulfonyl)-1H-pyrazolo[3,4-c]pyridin-1-yl]methyl}-1H-benzimidazol-1-yl)cyclohexanol; 3-(5-chloro-2-{[3-(methylsulfonyl)-6-oxido-1H-pyrazolo[3,4-c]pyridin-1-yl]methyl}-1H-benzimidazol-1-yl)cyclopentanol; 1-{[5-chloro-1-(pyrrolidin-3-yl)-1H-benzimidazol-2-yl]methyl}-3-(methylsulfonyl)-1H-pyrazolo[3,4-c]pyridine; 1-{[1-(azetidin-3-yl)-5-chloro-1H-benzimidazol-2-yl]methyl}-3-(methylsulfonyl)-1H-pyrazolo[3,4-c]pyridine; 1-{[5-chloro-1-(piperidin-4-yl)-1H-benzimidazol-2-yl]methyl}-3-(methylsulfonyl)-1H-indazole; 1-[3-(5-chloro-2-{[3-(methylsulfonyl)-1H-pyrazolo[3,4-c]pyridin-1-yl]methyl}-1H-benzimidazol-1-yl)pyrrolidin-1-yl]ethanone; 1-[3-(5-chloro-2-{[3-(methylsulfonyl)-1H-pyrazolo[3,4-c]pyridin-1-yl]methyl}-1H-benzimidazol-1-yl)pyrrolidin-1-yl]-2-hydroxyethanone; 2-amino-1-[3-(5-chloro-2-{[3-(methylsulfonyl)-1H-pyrazolo[3,4-c]pyridin-1-yl]methyl}-1H-benzimidazol-1-yl)pyrrolidin-1-yl]ethanone; 1-({5-chloro-1-[(35)-1-(2,2,2-trifluoroethyl)pyrrolidin-3-yl]-1H-benzimidazol-2-yl}methyl)-3-(methylsulfonyl)-1H-pyrazolo[3,4-c]pyridine; 1-({5-chloro-1-[(3R)-1-(2,2,2-trifluoroethyl)pyrrolidin-3-yl]-1H-benzimidazol-2-yl}methyl)-3-(methylsulfonyl)-1H-pyrazolo[3,4-c]pyridine; 1-{[5-chloro-1-(3,3,3-trifluoropropyl)-1H-benzimidazol-2-yl]methyl}-3-(methylsulfonyl)-1H-pyrazolo[3,4-c]pyridine; 1-{[5-chloro-1-(oxetan-3-ylmethyl)-1H-benzimidazol-2-yl]methyl}-3-(methylsulfonyl)-1H-pyrazolo[3,4-c]pyridine; 1-({5-chloro-1-[2-(oxetan-3-yl)ethyl]-1H-benzimidazol-2-yl}methyl)-3-(methylsulfonyl)-1H-pyrazolo[3,4-c]pyridine; 1-{[5-chloro-1-(6-fluoropyridin-3-yl)-1H-benzimidazol-2-yl]methyl}-3-(methylsulfonyl)-1H-indazole; 1-{[5-chloro-1-(6-fluoropyridin-3-yl)-1H-benzimidazol-2-yl]methyl}-3-(methylsulfonyl)-1H-pyrazolo[3,4-c]pyridine; 1-{[5-chloro-1-(6-fluoropyridin-3-yl)-1H-benzimidazol-2-yl]methyl}-3-(methylsulfonyl)-1H-pyrazolo[3,4-c]pyridine 6-oxide; 1-{[5-chloro-1-(6-methoxypyridin-3-yl)-1H-benzimidazol-2-yl]methyl}-3-(methylsulfonyl)-1H-indazole; 1-{[5-chloro-1-(6-chloropyridin-3-yl)-1H-benzimidazol-2-yl]methyl}-3-(methylsulfonyl)-1H-indazole; 1-{[5-chloro-1-(4,4,4-trifluorobutyl)-1H-benzimidazol-2-yl]methyl}-3-(methylsulfonyl)-1H-indazole; 1-{[5-chloro-1-(4,4,4-trifluorobutyl)-1H-benzimidazol-2-yl]methyl}-3-(methylsulfonyl)-1H-pyrazolo[3,4-c]pyridine 6-oxide; 1-{[5-chloro-1-(4,4,4-trifluorobutyl)-1H-benzimidazol-2-yl]methyl}-3-(methylsulfonyl)-1H-pyrazolo[3,4-c]pyridine; 1-{[5-chloro-7-fluoro-1-(3,3,3-trifluoropropyl)-1H-benzimidazol-2-yl]methyl}-3-(methylsulfonyl)-1H-pyrazolo[3,4-c]pyridine; 1-{[5-chloro-7-fluoro-1-(4,4,4-trifluorobutyl)-1H-benzimidazol-2-yl]methyl}-3-(methylsulfonyl)-1H-pyrazolo[3,4-c]pyridine; 1-{[5-chloro-1-(2-oxaspiro[33]hept-6-yl)-1H-benzimidazol-2-yl]methyl}-3-(methylsulfonyl)-1H-pyrazolo[3,4-c]pyridine; 1-({5-chloro-1-[2-(3-methyloxetan-3-yl)ethyl]-1H-benzimidazol-2-yl}methyl)-3-(methylsulfonyl)-1H-pyrazolo[3,4-c]pyridine; trans-3-(5-chloro-2-{[3-(methylsulfonyl)-1H-pyrazolo[3,4-c]pyridin-1-yl]methyl}-1H-benzimidazol-1-yl)-1-methylcyclobutanol; 3-(5-chloro-2-{[3-(methylsulfonyl)-1H-pyrazolo[3,4-c]pyridin-1-yl]methyl}-1H-benzimidazol-1-yl)propan-1-ol; 1-{[5-chloro-1-(tetrahydrofuran-3-yl)-1H-benzimidazol-2-yl]methyl}-3-(methylsulfonyl)-1H-pyrazolo[3,4-c]pyridine; 4-(5-chloro-2-{[3-(methylsulfonyl)-1H-pyrazolo[3,4-c]pyridin-1-yl]methyl}-1H-benzimidazol-1-yl)-2-methylbutan-2-ol; 4-(5-chloro-2-{[3-(methylsulfonyl)-1H-pyrazolo[3,4-c]pyridin-1-yl]methyl}-1H-benzimidazol-1-yl)butan-1-ol; 1-{[5-chloro-1-(tetrahydrofuran-3-ylmethyl)-1H-benzimidazol-2-yl]methyl}-3-(methylsulfonyl)-1H-pyrazolo[3,4-c]pyridine; trans-3-(5-chloro-2-{[3-(methylsulfonyl)-1H-pyrazolo[3,4-c]pyridin-1-yl]methyl}-1H-benzimidazol-1-yl)cyclobutanol; cis-3-(5-chloro-2-{[3-(methylsulfonyl)-1H-pyrazolo[3,4-c]pyridin-1-yl]methyl}-1H-benzimidazol-1-yl)-1-methylcyclobutanol; 1-[2-(5-chloro-2-{[3-(methylsulfonyl)-1H-pyrazolo[3,4-c]pyridin-1-yl]methyl}-1H-benzimidazol-1-yl)ethyl]cyclopropanol; 2-[2-(5-chloro-2-{[3-(methylsulfonyl)-1H-pyrazolo[3,4-c]pyridin-1-yl]methyl}-1H-benzimidazol-1-yl)ethoxy]ethanol; trans′-3-(5-chloro-2-{[3-(methylsulfonyl)-1H-pyrazolo[3,4-c]pyridin-1-yl]methyl}-1H-benzimidazol-1-yl)cyclopentanol; cis′-4-(5-chloro-2-{[3-(methylsulfonyl)-1H-pyrazolo[3,4-c]pyridin-1-yl]methyl}-1H-benzimidazol-1-yl)-1-methylcyclohexanol; 5-(5-chloro-2-{[3-(methylsulfonyl)-1H-pyrazolo[3,4-c]pyridin-1-yl]methyl}-1H-benzimidazol-1-yl)-2-methylpentan-2-ol; 2-[trans-3-(5-chloro-2-{[3-(methylsulfonyl)-1H-pyrazolo[3,4-c]pyridin-1-yl]methyl}-1H-benzimidazol-1-yl)cyclobutyl]propan-2-ol; 1-({5-chloro-1-[2-(morpholin-4-yl)ethyl]-1H-benzimidazol-2-yl}methyl)-3-(methylsulfonyl)-1H-pyrazolo[3,4-c]pyridine; trans-3-(5-chloro-2-{[3-(methylsulfonyl)-1H-pyrazolo[3,4-c]pyridin-1-yl]methyl}-1H-benzimidazol-1-yl)cyclobutanecarboxylic acid; 4-(5-chloro-2-{[3-(methylsulfonyl)-1H-pyrazolo[3,4-c]pyridin-1-yl]methyl}-1H-benzimidazol-1-yl)-1,1,1-trifluorobutan-2-ol; cis-3-(5-chloro-2-{[3-(methylsulfonyl)-1H-pyrazolo[3,4-c]pyridin-1-yl]methyl}-1H-benzimidazol-1-yl)-1-methylcyclopentanol; 4-(5-chloro-2-{[3-(methylsulfonyl)-1H-pyrazolo[3,4-c]pyridin-1-yl]methyl}-1H-benzimidazol-1-yl)-1,1-difluorobutan-2-ol; trans′-4-(5-chloro-2-{[3-(methylsulfonyl)-1H-pyrazolo[3,4-c]pyridin-1-yl]methyl}-1H-benzimidazol-1-yl)cyclopentane-1,2-diol; trans′-3-(5-chloro-2-{[3-(methylsulfonyl)-1H-pyrazolo[3,4-c]pyridin-1-yl]methyl}-1H-benzimidazol-1-yl)-1-(hydroxymethyl)cyclobutanol; 1-[(3R)-3-(5-chloro-2-{[3-(methylsulfonyl)-1H-pyrazolo[3,4-c]pyridin-1-yl]methyl}-1H-benzimidazol-1-yl)pyrrolidin-1-yl]ethanone; 1-[3-(5-chloro-2-{[3-(methylsulfonyl)-1H-indazol-1-yl]methyl}-1H-benzimidazol-1-yl)pyrrolidin-1-yl]ethanone; 1-[(3R)-3-(5-chloro-2-{[3-(methylsulfonyl)-1H-indazol-1-yl]methyl}-1H-benzimidazol-1-yl)pyrrolidin-1-yl]propan-1-one; 1-[(3R)-3-(5-chloro-2-{[3-(methylsulfonyl)-1H-pyrazolo[3,4-c]pyridin-1-yl]methyl}-1H-benzimidazol-1-yl)pyrrolidin-1-yl]-2-methylpropan-1-one; 1-[(3R)-3-(5-chloro-2-{[3-(methylsulfonyl)-1H-pyrazolo[3,4-c]pyridin-1-yl]methyl}-1H-benzimidazol-1-yl)pyrrolidin-1-yl]-2-hydroxy-2-methylpropan-1-one; 1-({5-chloro-1-[(3R)-1-(methylsulfonyl)pyrrolidin-3-yl]-1H-benzimidazol-2-yl}methyl)-3-(methylsulfonyl)-1H-pyrazolo[3,4-c]pyridine; 2-[(3R)-3-(5-chloro-2-{[3-(methylsulfonyl)-1H-pyrazolo[3,4-c]pyridin-1-yl]methyl}-1H-benzimidazol-1-yl)pyrrolidin-1-yl]ethanol; 4-(5-chloro-2-{[3-(methylsulfonyl)-1H-pyrazolo[3,4-c]pyridin-1-yl]methyl}-1H-benzimidazol-1-yl)pyrrolidin-2-one; 1-{[5-chloro-1-(2-oxa-5-azaspiro[3.4]oct-7-yl)-1H-benzimidazol-2-yl]methyl}-3-(methylsulfonyl)-1H-pyrazolo[3,4-c]pyridine; 1-({5-chloro-1-[2-(methylsulfonyl)ethyl]-1H-indol-2-yl}methyl)-3-(methylsulfonyl)-1H-pyrazolo[3,4-c]pyridine; 1-({5-chloro-1-[3-(methylsulfonyl)propyl]-1H-indol-2-yl}methyl)-3-(methylsulfonyl)-1H-pyrazolo[3,4-c]pyridine; 1-({5-chloro-7-fluoro-1-[2-(methylsulfonyl)ethyl]-1H-indol-2-yl}methyl)-3-(methylsulfonyl)-1H-pyrazolo[3,4-c]pyridine; 1-[2-(5-chloro-2-{[3-(methylsulfonyl)-1H-pyrazolo[3,4-c]pyridin-1-yl]methyl}-1H-benzimidazol-1-yl)ethyl]pyrrolidin-3-01; 1-[2-(5-chloro-2-{[3-(methylsulfonyl)-1H-pyrazolo[3,4-c]pyridin-1-yl]methyl}-1H-benzimidazol-1-yl)ethyl]piperidin-4-ol; [trans′-3-(5-chloro-2-{[3-(methylsulfonyl)-1H-pyrazolo[3,4-c]pyridin-1-yl]methyl}-1H-benzimidazol-1-yl)cyclobutyl]methanol; 1-({5-chloro-1-[(3R)-1,1-dioxidotetrahydrothiophen-3-yl]-7-fluoro-1H-benzimidazol-2-yl}methyl)-3-(methylsulfonyl)-1H-pyrazolo[3,4-c]pyridine; 3-(5-chloro-2-{[3-(methylsulfonyl)-1H-pyrazolo[3,4-c]pyridin-1-yl]methyl}-1H-benzimidazol-1-yl)(1,1-H₂)propan-1-ol; 4-(5-chloro-2-{[3-(methylsulfonyl)-1H-pyrazolo[3,4-c]pyridin-1-yl]methyl}-1H-benzimidazol-1-yl)-1,1,1-trifluoro-2-methylbutan-2-ol; 1-{(JR)-1-[5-chloro-1-(3,3,3-trifluoropropyl)-1H-benzimidazol-2-yl]ethyl}-3-(methylsulfonyl)-1H-pyrazolo[3,4-c]pyridine; and 1-{(1S)-1-[5-chloro-1-(3,3,3-trifluoropropyl)-1H-benzimidazol-2-yl]ethyl}-3-(methylsulfonyl)-1H-pyrazolo[3,4-c]pyridine.

### Compounds of Formula (B17)

Compounds of the general Formula (B17) are described in PCT Publication No. WO 2011/005842, published Jan. 13, 2011, which is hereby incorporated by reference in its entirety. Formula (B17) has the structure:

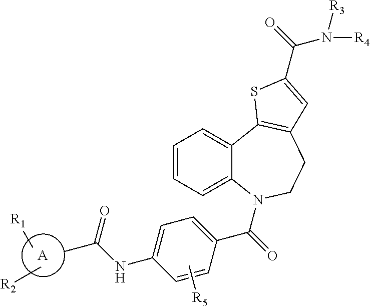

or a pharmaceutically acceptable salt thereof, wherein: A can be aryl or heteroaryl; R₁ can be alkyl, alkoxy, haloalkyl, aryl, heteroaryl, heterocyclyl, cycloalkyl, said heterocyclyl is optionally substituted by one to three substituents independently selected from halo, hydroxyl, haloalkyl, alkoxy, alkyl, alkoxy-alkyl-, hydroxyl-alkyl-, CN, alky-NH—; said aryl or heteroaryl can be optionally substituted by one to three substituents independently selected from halo, cyano, nitro, hydroxyl, alkyl, alkoxy, alkyl-NH—, with the proviso that when A is aryl, R₁ is not unsubstituted aryl; R₂ can be hydrogen, alkyl, alkoxy, amino, alkyl-NH—, CN, alkyl-SO₂—, or halo; R₃ can be hydrogen, alkyl, heterocyclyl, heteroaryl, heteroaryl-alkyl-, or cycloalkyl, said alkyl is optionally substituted by one substituent selected from NH₂—C(O)—, halo, hydroxyl, NH₂—SO₂—, alkoxy-alkyl-, heterocyclyl; aryl, heteroaryl, CN, alkyl-NH—; R₄ can be hydrogen, or alkyl; or haloalkyl; R₃ and R₄ can be taken together with the nitrogen atom to which they are attached optionally form a 3- to 7-membered ring; R₅ can be hydrogen, alkyl, alkoxy, haloalkyl, or halo.

Examples of Compounds of Formula (B17) include:

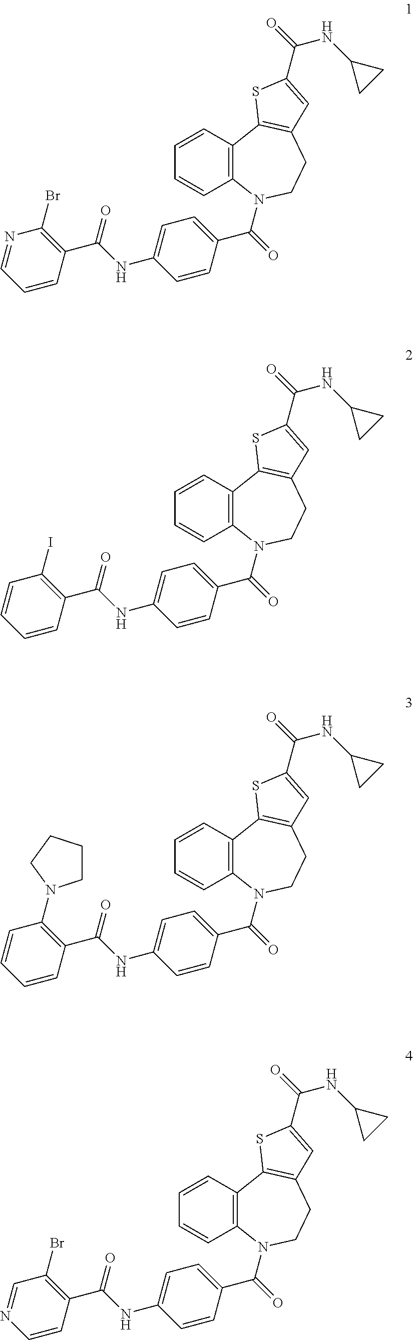 [SMILES: O=C(NC1CC1)c1cc2c(s1)-c1ccccc1N(C(=O)c1ccc(NC(=O)c3cccnc3Br)cc1)CC2 ; O=C(NC1CC1)c1cc2c(s1)-c1ccccc1N(C(=O)c1ccc(NC(=O)c3ccccc3I)cc1)CC2 ; O=C(NC1CC1)c1cc2c(s1)-c1ccccc1N(C(=O)c1ccc(NC(=O)c3ccccc3N3CCCC3)cc1)CC2 ; O=C(NC1CC1)c1cc2c(s1)-c1ccccc1N(C(=O)c1ccc(NC(=O)c3ccncc3Br)cc1)CC2]

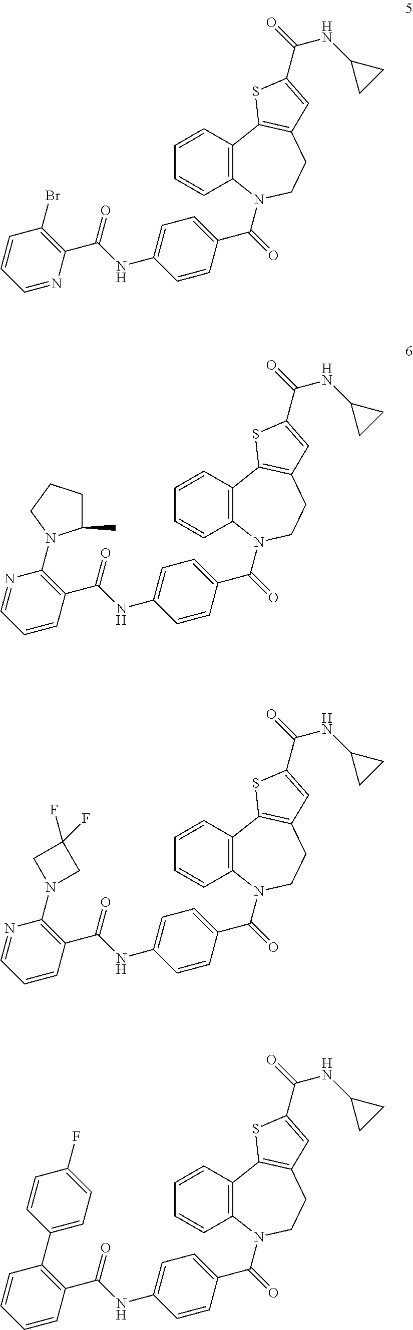 [SMILES: O=C(NC1CC1)c1cc2c(s1)-c1ccccc1N(C(=O)c1ccc(NC(=O)c3ncccc3Br)cc1)CC2 ; C[C@@H]1CCCN1c1ncccc1C(=O)Nc1ccc(C(=O)N2CCc3cc(C(=O)NC4CC4)sc3-c3ccccc32)cc1 ; O=C(NC1CC1)c1cc2c(s1)-c1ccccc1N(C(=O)c1ccc(NC(=O)c3cccnc3N3CC(F)(F)C3)cc1)CC2 ; O=C(NC1CC1)c1cc2c(s1)-c1ccccc1N(C(=O)c1ccc(NC(=O)c3ccccc3-c3ccc(F)cc3)cc1)CC2]

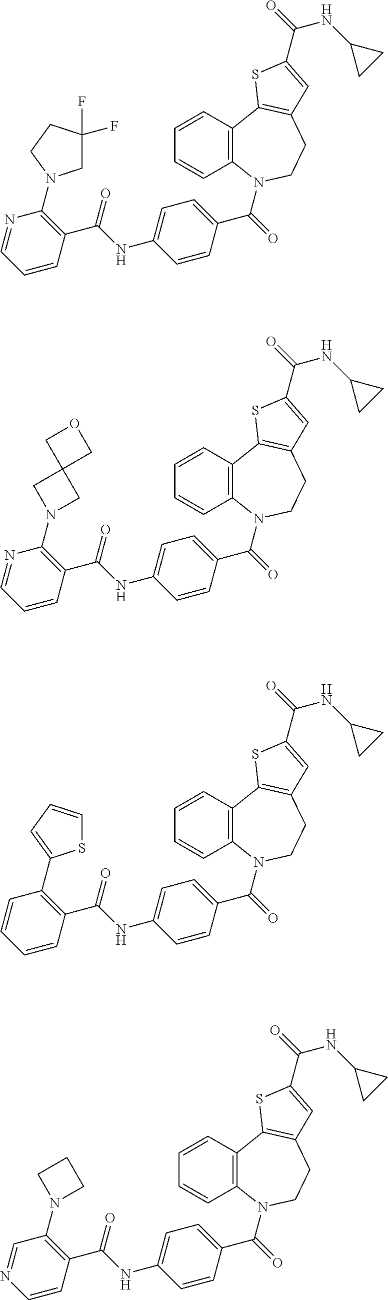 [SMILES: O=C(NC1CC1)c1cc2c(s1)-c1ccccc1N(C(=O)c1ccc(NC(=O)c3cccnc3N3CCC(F)(F)C3)cc1)CC2 ; O=C(NC1CC1)c1cc2c(s1)-c1ccccc1N(C(=O)c1ccc(NC(=O)c3cccnc3N3CC4(COC4)C3)cc1)CC2 ; O=C(NC1CC1)c1cc2c(s1)-c1ccccc1N(C(=O)c1ccc(NC(=O)c3ccccc3-c3cccs3)cc1)CC2 ; O=C(NC1CC1)c1cc2c(s1)-c1ccccc1N(C(=O)c1ccc(NC(=O)c3ccncc3N3CCC3)cc1)CC2]

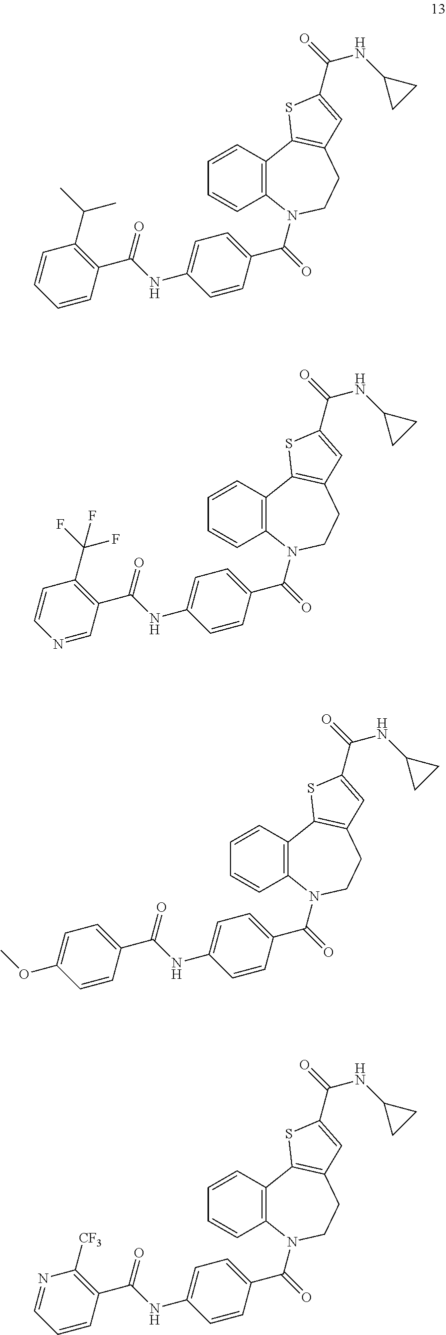 [SMILES: CC(C)c1ccccc1C(=O)Nc1ccc(C(=O)N2CCc3cc(C(=O)NC4CC4)sc3-c3ccccc32)cc1 ; O=C(NC1CC1)c1cc2c(s1)-c1ccccc1N(C(=O)c1ccc(NC(=O)c3cnccc3C(F)(F)F)cc1)CC2 ; COc1ccc(C(=O)Nc2ccc(C(=O)N3CCc4cc(C(=O)NC5CC5)sc4-c4ccccc43)cc2)cc1 ; O=C(NC1CC1)c1cc2c(s1)-c1ccccc1N(C(=O)c1ccc(NC(=O)c3cccnc3C(F)(F)F)cc1)CC2]

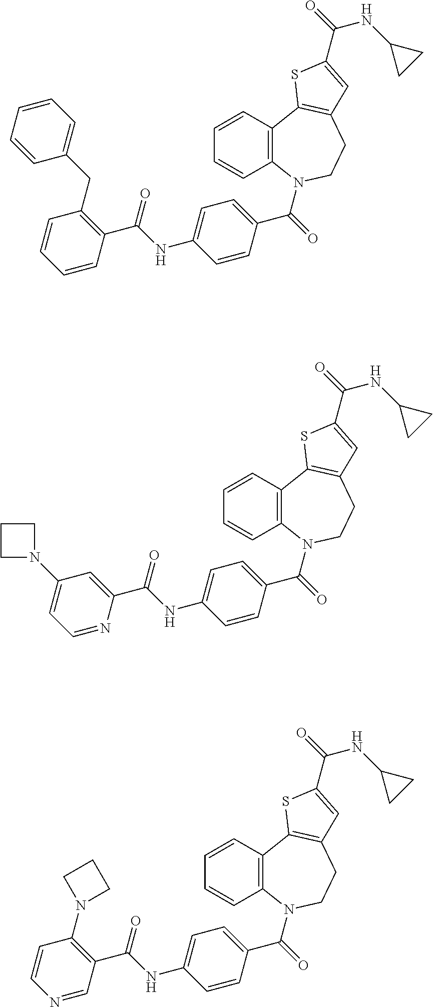 [SMILES: O=C(NC1CC1)c1cc2c(s1)-c1ccccc1N(C(=O)c1ccc(NC(=O)c3ccccc3Cc3ccccc3)cc1)CC2 ; O=C(Nc1ccc(C(=O)N2CCc3cc(C(=O)NC4CC4)sc3-c3ccccc32)cc1)c1cc(N2CCC2)ccn1 ; O=C(NC1CC1)c1cc2c(s1)-c1ccccc1N(C(=O)c1ccc(NC(=O)c3cnccc3N3CCC3)cc1)CC2]

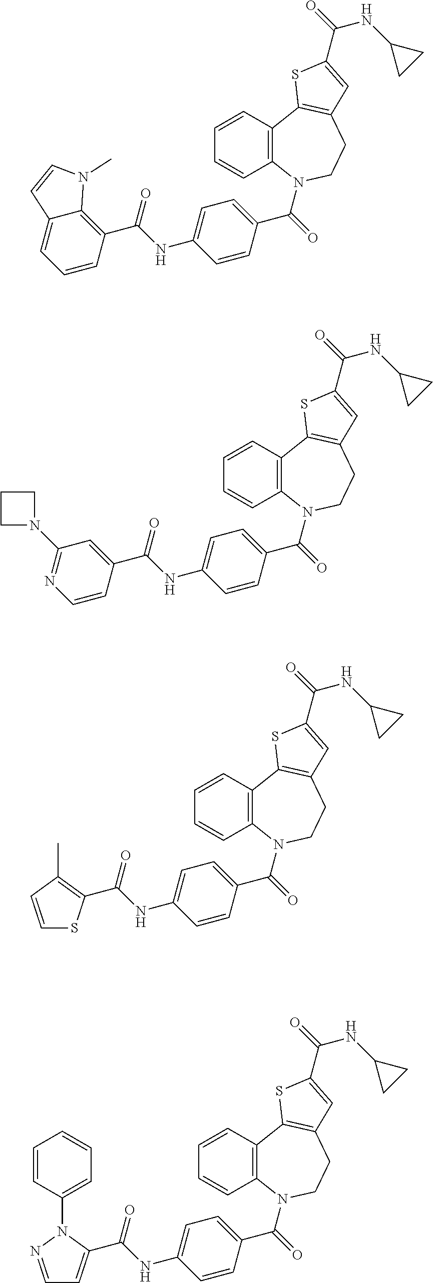 [SMILES: Cn1ccc2cccc(C(=O)Nc3ccc(C(=O)N4CCc5cc(C(=O)NC6CC6)sc5-c5ccccc54)cc3)c21 ; O=C(Nc1ccc(C(=O)N2CCc3cc(C(=O)NC4CC4)sc3-c3ccccc32)cc1)c1ccnc(N2CCC2)c1 ; Cc1ccsc1C(=O)Nc1ccc(C(=O)N2CCc3cc(C(=O)NC4CC4)sc3-c3ccccc32)cc1 ; O=C(NC1CC1)c1cc2c(s1)-c1ccccc1N(C(=O)c1ccc(NC(=O)c3ccnn3-c3ccccc3)cc1)CC2]

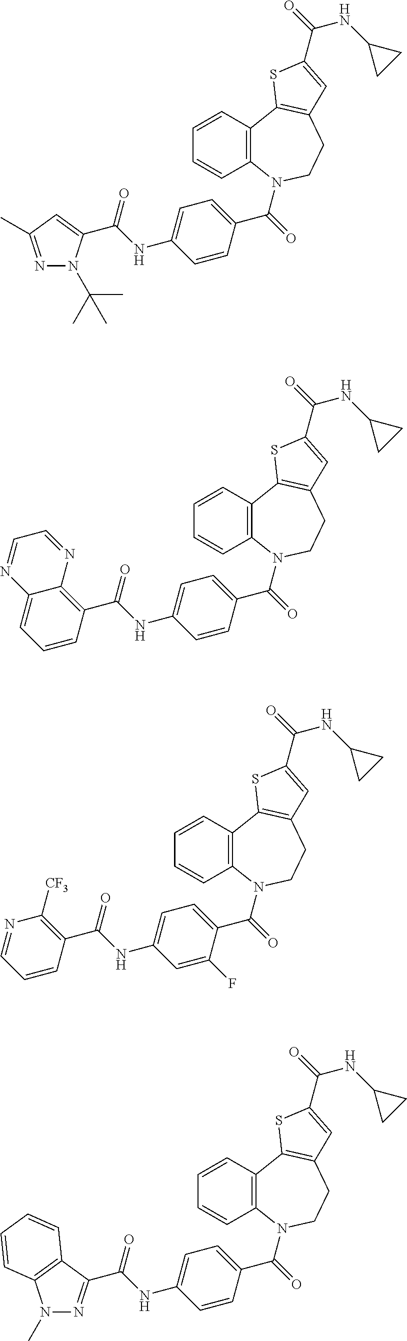 [SMILES: Cc1cc(C(=O)Nc2ccc(C(=O)N3CCc4cc(C(=O)NC5CC5)sc4-c4ccccc43)cc2)n(C(C)(C)C)n1 ; O=C(NC1CC1)c1cc2c(s1)-c1ccccc1N(C(=O)c1ccc(NC(=O)c3cccc4nccnc34)cc1)CC2 ; O=C(NC1CC1)c1cc2c(s1)-c1ccccc1N(C(=O)c1ccc(NC(=O)c3cccnc3C(F)(F)F)cc1F)CC2 ; Cn1nc(C(=O)Nc2ccc(C(=O)N3CCc4cc(C(=O)NC5CC5)sc4-c4ccccc43)cc2)c2ccccc21]

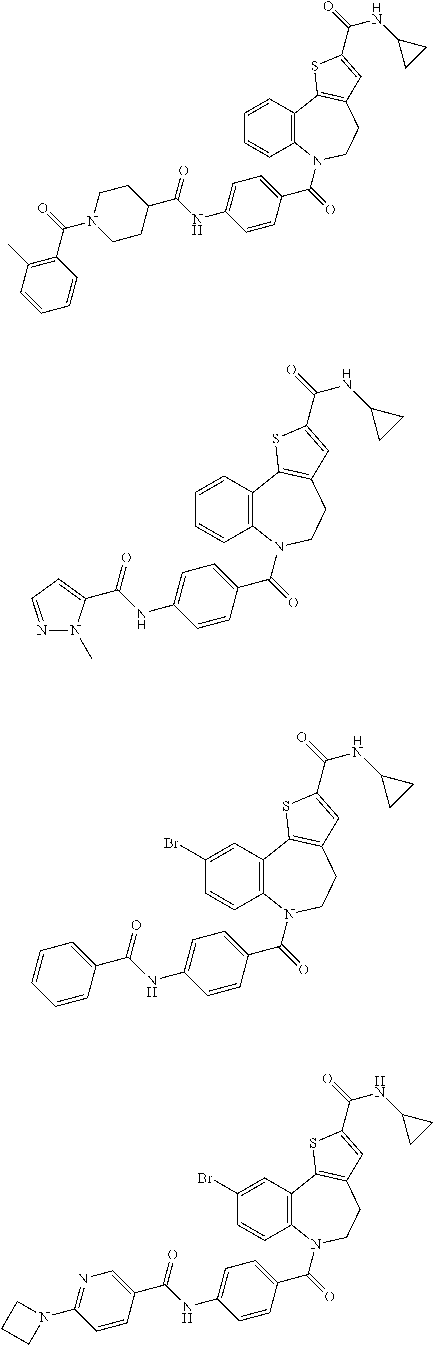 [SMILES: Cc1ccccc1C(=O)N1CCC(C(=O)Nc2ccc(C(=O)N3CCc4cc(C(=O)NC5CC5)sc4-c4ccccc43)cc2)CC1 ; Cn1nccc1C(=O)Nc1ccc(C(=O)N2CCc3cc(C(=O)NC4CC4)sc3-c3ccccc32)cc1 ; O=C(Nc1ccc(C(=O)N2CCc3cc(C(=O)NC4CC4)sc3-c3cc(Br)ccc32)cc1)c1ccccc1 ; O=C(Nc1ccc(C(=O)N2CCc3cc(C(=O)NC4CC4)sc3-c3cc(Br)ccc32)cc1)c1ccc(N2CCC2)nc1]

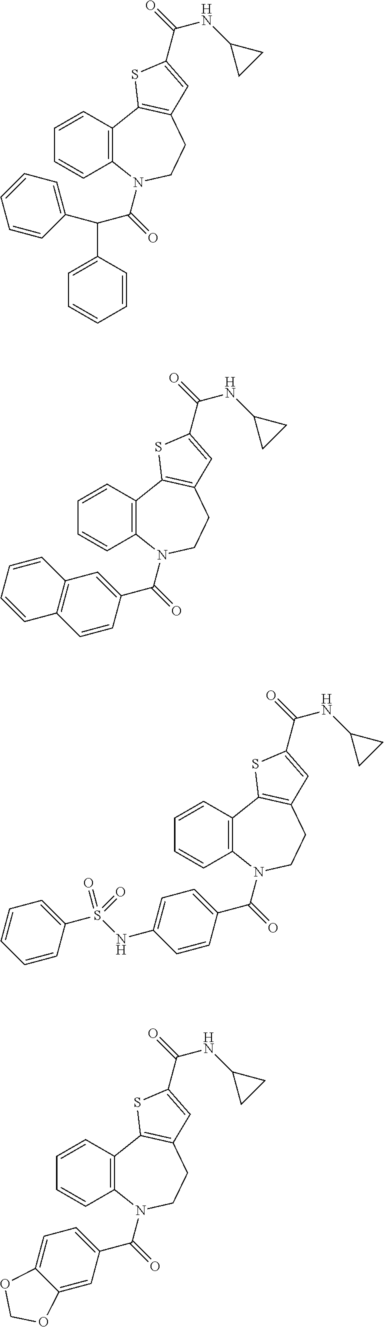 [SMILES: O=C(NC1CC1)c1cc2c(s1)-c1ccccc1N(C(=O)C(c1ccccc1)c1ccccc1)CC2 ; O=C(NC1CC1)c1cc2c(s1)-c1ccccc1N(C(=O)c1ccc3ccccc3c1)CC2 ; O=C(NC1CC1)c1cc2c(s1)-c1ccccc1N(C(=O)c1ccc(NS(=O)(=O)c3ccccc3)cc1)CC2 ; O=C(NC1CC1)c1cc2c(s1)-c1ccccc1N(C(=O)c1ccc3c(c1)OCO3)CC2]

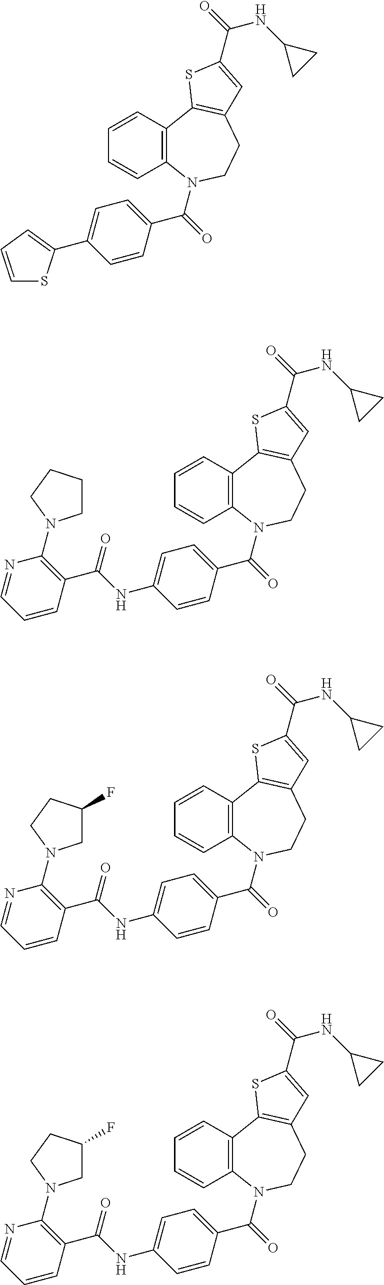 [SMILES: O=C(NC1CC1)c1cc2c(s1)-c1ccccc1N(C(=O)c1ccc(-c3cccs3)cc1)CC2 ; O=C(NC1CC1)c1cc2c(s1)-c1ccccc1N(C(=O)c1ccc(NC(=O)c3cccnc3N3CCCC3)cc1)CC2 ; O=C(NC1CC1)c1cc2c(s1)-c1ccccc1N(C(=O)c1ccc(NC(=O)c3cccnc3N3CC[C@@H](F)C3)cc1)CC2 ; O=C(NC1CC1)c1cc2c(s1)-c1ccccc1N(C(=O)c1ccc(NC(=O)c3cccnc3N3CC[C@H](F)C3)cc1)CC2]

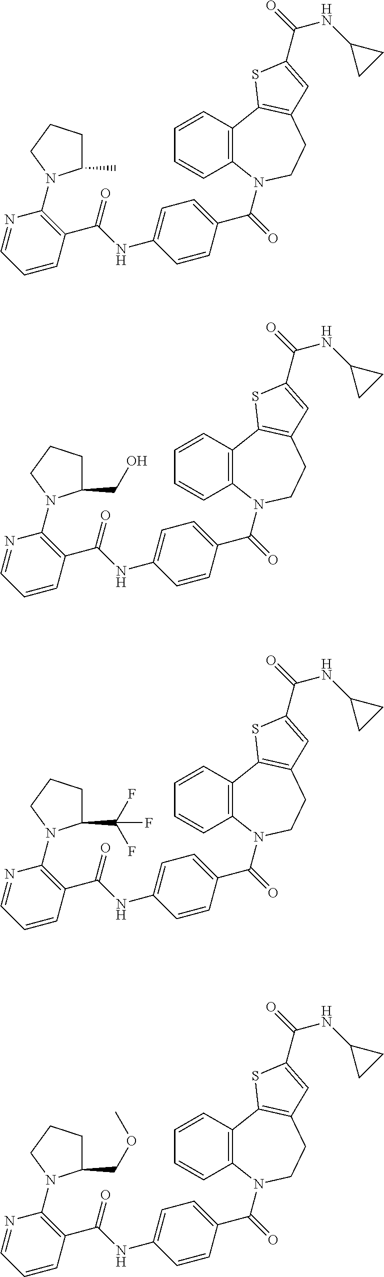 [SMILES: C[C@H]1CCCN1c1ncccc1C(=O)Nc1ccc(C(=O)N2CCc3cc(C(=O)NC4CC4)sc3-c3ccccc32)cc1 ; O=C(NC1CC1)c1cc2c(s1)-c1ccccc1N(C(=O)c1ccc(NC(=O)c3cccnc3N3CCC[C@H]3CO)cc1)CC2 ; O=C(NC1CC1)c1cc2c(s1)-c1ccccc1N(C(=O)c1ccc(NC(=O)c3cccnc3N3CCC[C@H]3C(F)(F)F)cc1)CC2 ; COC[C@@H]1CCCN1c1ncccc1C(=O)Nc1ccc(C(=O)N2CCc3cc(C(=O)NC4CC4)sc3-c3ccccc32)cc1]

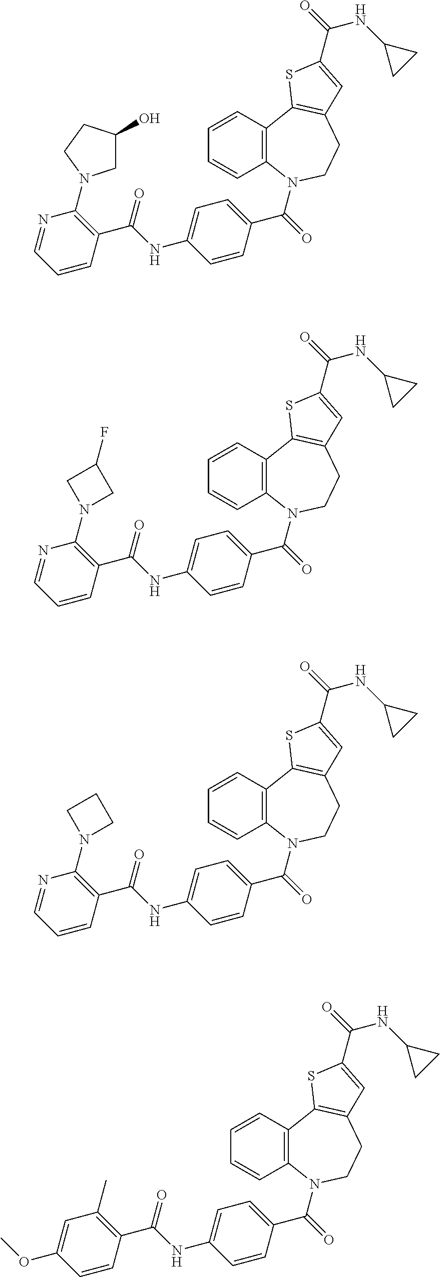 [SMILES: O=C(NC1CC1)c1cc2c(s1)-c1ccccc1N(C(=O)c1ccc(NC(=O)c3cccnc3N3CC[C@@H](O)C3)cc1)CC2 ; O=C(NC1CC1)c1cc2c(s1)-c1ccccc1N(C(=O)c1ccc(NC(=O)c3cccnc3N3CC(F)C3)cc1)CC2 ; O=C(NC1CC1)c1cc2c(s1)-c1ccccc1N(C(=O)c1ccc(NC(=O)c3cccnc3N3CCC3)cc1)CC2 ; COc1ccc(C(=O)Nc2ccc(C(=O)N3CCc4cc(C(=O)NC5CC5)sc4-c4ccccc43)cc2)c(C)c1]

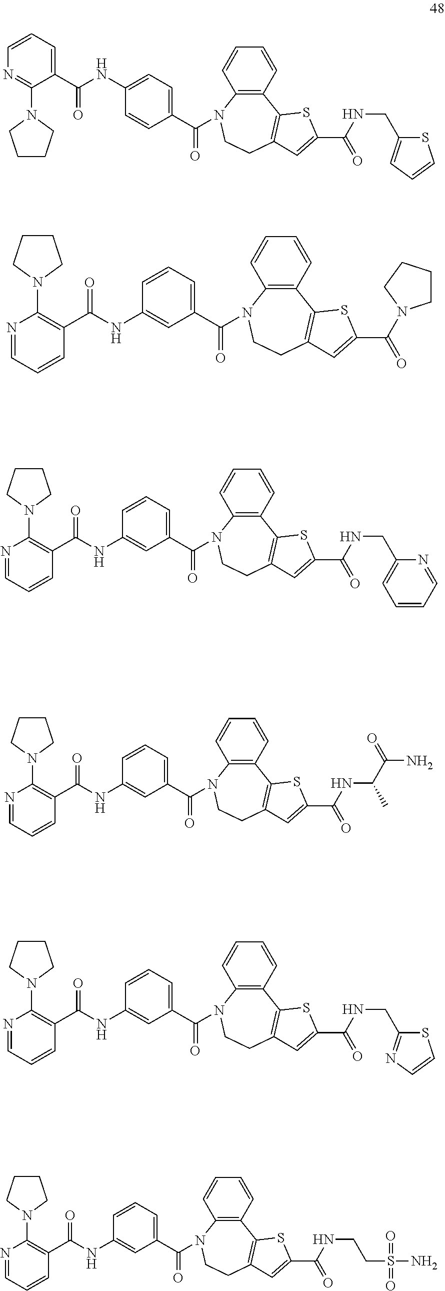 [SMILES: O=C(NCc1cccs1)c1cc2c(s1)-c1ccccc1N(C(=O)c1ccc(NC(=O)c3cccnc3N3CCCC3)cc1)CC2 ; O=C(Nc1cccc(C(=O)N2CCc3cc(C(=O)N4CCCC4)sc3-c3ccccc32)c1)c1cccnc1N1CCCC1 ; O=C(NCc1ccccn1)c1cc2c(s1)-c1ccccc1N(C(=O)c1cccc(NC(=O)c3cccnc3N3CCCC3)c1)CC2 ; C[C@H](NC(=O)c1cc2c(s1)-c1ccccc1N(C(=O)c1cccc(NC(=O)c3cccnc3N3CCCC3)c1)CC2)C(N)=O ; O=C(NCc1nccs1)c1cc2c(s1)-c1ccccc1N(C(=O)c1cccc(NC(=O)c3cccnc3N3CCCC3)c1)CC2 ; NS(=O)(=O)CCNC(=O)c1cc2c(s1)-c1ccccc1N(C(=O)c1cccc(NC(=O)c3cccnc3N3CCCC3)c1)CC2]

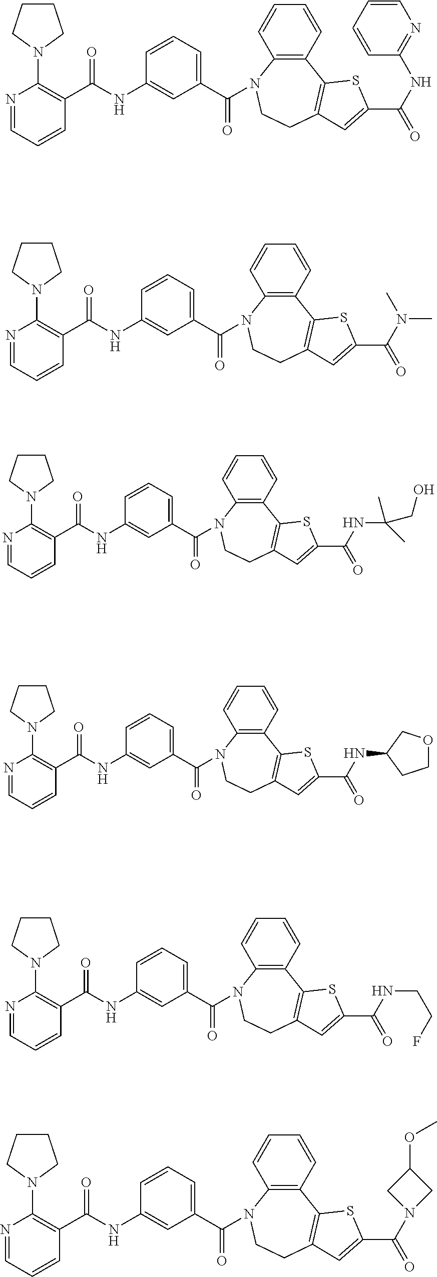 [SMILES: O=C(Nc1ccccn1)c1cc2c(s1)-c1ccccc1N(C(=O)c1cccc(NC(=O)c3cccnc3N3CCCC3)c1)CC2 ; CN(C)C(=O)c1cc2c(s1)-c1ccccc1N(C(=O)c1cccc(NC(=O)c3cccnc3N3CCCC3)c1)CC2 ; CC(C)(CO)NC(=O)c1cc2c(s1)-c1ccccc1N(C(=O)c1cccc(NC(=O)c3cccnc3N3CCCC3)c1)CC2 ; O=C(N[C@@H]1CCOC1)c1cc2c(s1)-c1ccccc1N(C(=O)c1cccc(NC(=O)c3cccnc3N3CCCC3)c1)CC2 ; O=C(NCCF)c1cc2c(s1)-c1ccccc1N(C(=O)c1cccc(NC(=O)c3cccnc3N3CCCC3)c1)CC2 ; COC1CN(C(=O)c2cc3c(s2)-c2ccccc2N(C(=O)c2cccc(NC(=O)c4cccnc4N4CCCC4)c2)CC3)C1]

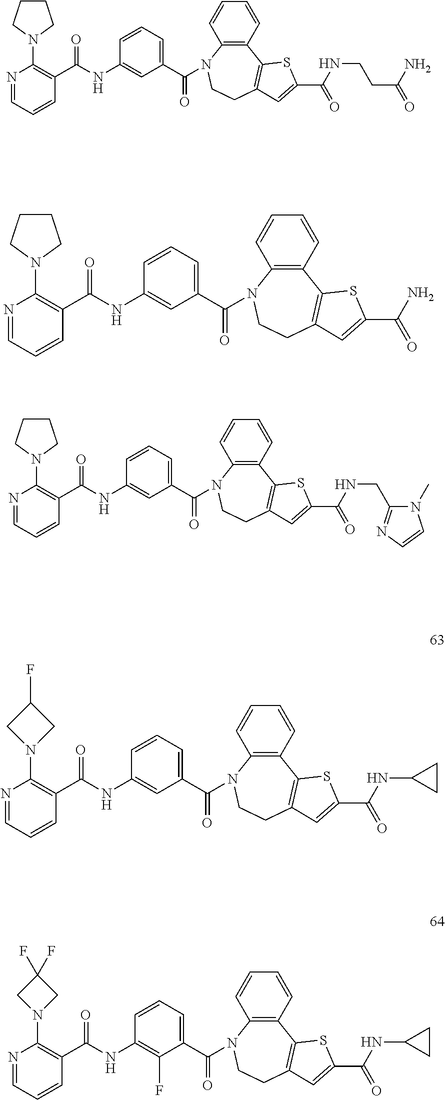 [SMILES: NC(=O)CCNC(=O)c1cc2c(s1)-c1ccccc1N(C(=O)c1cccc(NC(=O)c3cccnc3N3CCCC3)c1)CC2 ; NC(=O)c1cc2c(s1)-c1ccccc1N(C(=O)c1cccc(NC(=O)c3cccnc3N3CCCC3)c1)CC2 ; Cn1ccnc1CNC(=O)c1cc2c(s1)-c1ccccc1N(C(=O)c1cccc(NC(=O)c3cccnc3N3CCCC3)c1)CC2 ; O=C(NC1CC1)c1cc2c(s1)-c1ccccc1N(C(=O)c1cccc(NC(=O)c3cccnc3N3CC(F)C3)c1)CC2 ; O=C(NC1CC1)c1cc2c(s1)-c1ccccc1N(C(=O)c1cccc(NC(=O)c3cccnc3N3CC(F)(F)C3)c1F)CC2]

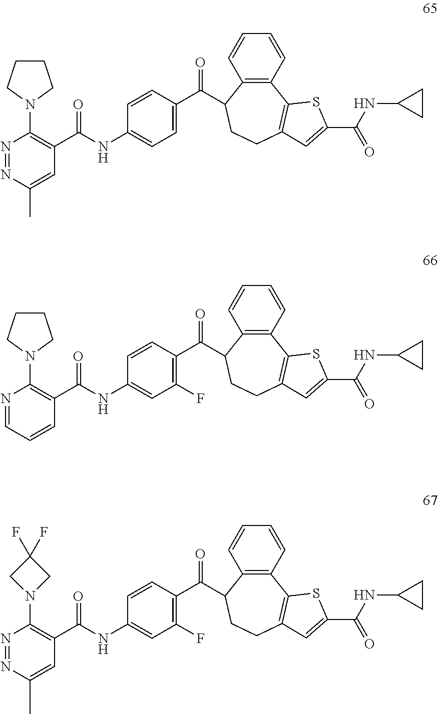 [SMILES: Cc1cc(C(=O)Nc2ccc(C(=O)C3CCc4cc(C(=O)NC5CC5)sc4-c4ccccc43)cc2)c(N2CCCC2)nn1 ; O=C(NC1CC1)c1cc2c(s1)-c1ccccc1C(C(=O)c1ccc(NC(=O)c3cccnc3N3CCCC3)cc1F)CC2 ; Cc1cc(C(=O)Nc2ccc(C(=O)C3CCc4cc(C(=O)NC5CC5)sc4-c4ccccc43)c(F)c2)c(N2CC(F)(F)C2)nn1]

### Compounds of Formula (B18)

Compounds of the general Formula (B18) are described in U.S. Publication No. 2013/0273037, published Oct. 17, 2013, which is hereby incorporated by reference in its entirety. Formula (B18) has the structure:

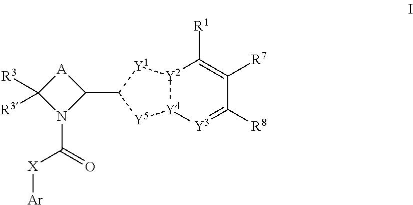

or a pharmaceutically acceptable salt thereof, wherein: a) Y¹ can be N, NH or CH, Y² is C, Y³ is N or CR^{8′}, Y⁴ is N or C and Y⁵ is N, NR^{2′} or CR², wherein at least two of Y¹, Y², Y³, Y⁴ and Y⁵ are independently N, NH or NR^{2′}; or b) Y¹ can be N, NH or CH, Y² is N or C, Y³ is N or CR^{8′}, Y⁴ is N or C, and Y⁵ is N or NR^{2′}, wherein at least two of Y¹, Y², Y³, Y⁴ and Y⁵ are independently N, NH or NR^{2′}; or c) Y¹ can be N, NH or CH, Y² is N or C, Y³ is CR^{8′}, Y⁴ is N or C, and Y⁵ is N, NR^{2′} or CR², wherein at least two of Y¹, Y², Y³, Y⁴ and Y⁵ are independently N, NH or NR^{2′}; the dashed bonds ---- can be selected from single bonds and double bonds so as to provide an aromatic ring system; A can be —(CR⁴R^{4′})ₙ— wherein any one CR⁴R^{4′} of said —(CR⁴R^{4′})ₙ— may be optionally replaced with —O—, —S—, —S(O)ₚ—, NH or NR^{a}; n can be 3, 4, 5 or 6; each p can be 1 or 2; Ar can be a C₂-C₂₀ heterocyclyl group or a C₆-C₂₀ aryl group, wherein the C₂-C₂₀ heterocyclyl group or the C₆-C₂₀ aryl group is optionally substituted with 1 to 5 R⁶; X can be —C(R¹³)(R¹⁴)—, —N(CH₂R¹⁴)— or —NH—, or X is absent; R¹ can be H, —OR¹¹, —NR¹¹R¹², —NR¹¹C(O)R¹¹, —NR¹¹C(O)OR¹¹, —NR¹¹C(O)NR¹¹R¹², N₃, CN, NO₂, —R¹¹, —S(O)ₚR^{a}, NR¹¹S(O)ₚR^{a}, —C(═O)R¹¹, —C(═O)OR¹¹, —C(═O)NR¹¹R¹², —C(═O)SR¹¹, —S(O)ₚ(OR¹¹), —SO₂NR¹¹R¹², —NR¹¹S(O)ₚ(OR¹¹), —NR¹¹SOₚNR¹¹R¹², —NR¹¹C(═NR¹¹)NR¹¹R¹², halogen, C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; R² can be H, CN, NO₂, halogen or (C₁-C₈)alkyl; R^{2′} can be H or (C₁-C₈)alkyl; R³ can be H, —OR¹¹, —NR¹¹R¹², —NR¹¹C(O)R¹¹, —NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(═O)OR¹¹, —C(═O)NR¹¹R¹², —C(═O)SR¹¹, —S(O)ₚ(OR¹¹), —SO₂NR¹¹R¹², —NR¹¹S(O)ₚ(OR¹¹), —NR¹¹SOₚNR¹¹R¹², —NR¹¹C(═NR¹¹)NR¹¹R¹², halogen, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; R^{3′} can be H, —OR¹¹, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; each R⁴ can be independently H, —OR¹¹, —NR¹¹R¹², —NR¹¹C(O)R¹¹, —NR¹¹C(O)OR¹¹, —NR¹¹C(O)NR¹¹R¹², N₃, CN, NO₂, SR¹¹, —S(O)ₚR^{a}, —NR¹¹S(O)ₚR^{a}, —C(═O)R¹¹, —C(═O)OR¹¹, —C(═O)NR¹¹R¹², —C(═O)SR¹¹—S(O)ₚ(OR¹¹), —SO₂NR¹¹R¹², —NR¹¹S(O)ₚ(OR¹¹), —NR¹¹SOₚNR¹¹R¹², NR¹¹C(═NR¹¹)NR¹¹R¹², halogen, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; and each R^{4′} can be independently H, OR¹¹, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; or two R⁴ on adjacent carbon atoms, when taken together, may form a double bond between the two carbons to which they are attached or may form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; or two R⁴ on non-adjacent carbon atoms, when taken together, may form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; or two R⁴ and two R^{4′} on adjacent carbon atoms, when taken together, may form an optionally substituted C₆ aryl ring; or one R⁴ and one R^{4′} on the same carbon atom, when taken together, may form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; each R⁵ can be independently H, —OR¹¹, —NR¹¹R¹², —NR¹¹C(O)R¹¹, —NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(═O)OR¹¹, —C(═O)NR¹¹R¹², —C(═O)SR¹¹, —S(O)ₚ(OR¹¹), —SO₂NR¹¹R¹², —NR¹¹S(O)ₚ(OR¹¹), —NR¹¹SOₚNR¹¹R¹², —NR¹¹C(═NR¹¹)NR¹¹R¹², halogen, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; each R^{5′} can be independently H, —OR¹¹, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; each R⁶ can be independently H, oxo, —OR¹¹, —NR¹¹R¹², NR¹¹C(O)R¹¹, NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(═O)OR¹¹, —C(═O)SR¹¹, —S(O)ₚ(OR¹¹), —SO₂NR¹¹R¹², —NR¹¹S(O)ₚ(OR¹¹), —NR¹¹SOₚNR¹¹R¹², —NR¹¹C(═NR¹¹)NR¹¹R¹², halogen, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; or two R⁶ on adjacent carbon atoms, when taken together, may form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; or any R⁶ adjacent to the obligate carbonyl group of said Ar, when taken together with R³, may form a bond or a —(CR⁵R^{5′})ₘ— group wherein m is 1 or 2; or any R⁶ adjacent to the obligate carbonyl group of said Ar, when taken together with R² or R^{2′} may form a bond; R⁷ can be H, —OR¹¹, —NR¹¹R¹², —NR¹¹C(O)R¹¹, —NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(═O)OR¹¹, —C(═O)NR¹¹R¹², —C(═O)SR¹¹, —S(O)ₚ(OR¹¹), —SO₂NR¹¹R¹², —NR¹¹S(O)ₚ(OR¹¹), —NR¹¹SOₚNR¹¹R¹², NR C(═NR¹¹)NR¹¹R¹², halogen, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; R⁸ can be H, —OR¹¹, —NR¹¹R¹², —NR¹¹C(O)R¹¹, NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(═O)OR¹¹, —C(═O)SR¹¹, —S(O)ₚ(OR¹¹), —SO₂NR¹¹R¹², —NR¹¹S(O)ₚ(OR¹¹), —NR¹¹SOₚNR¹¹R¹², NR¹¹C(═NR¹¹)NR¹¹R¹², halogen, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; R^{8′} can be H, —OR¹¹, —NR¹¹R¹², —NR¹¹C(O)R¹¹, —NR¹¹C(O)OR¹¹, —NR¹¹C(O)NR¹¹R¹², N₃, CN, NO₂, —SR¹¹, —S(O)ₚR^{a}, —NR¹¹S(O)ₚR^{a}, —C(═O)R¹¹, —C(C═O)OR¹¹, —C(═O)OR¹¹, —C(═O)SR¹¹, —S(O)ₚ(OR¹¹), —SO₂NR¹¹R¹², —NR¹¹S(O)ₚ(OR¹¹), —NR¹¹SOₚNR¹¹R¹², —NR¹¹C(═NR¹¹)NR¹¹R¹², halogen, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl; each R^{a} can be independently (C₁-C₈)alkyl, (C₁-C₈)haloalkyl, (C₂-C₈)alkenyl, (C₂-C₈)alkynyl, aryl(C₁-C₈)alkyl C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl wherein any (C₁-C₈)alkyl, (C₁-C₈)haloalkyl, (C₂-C₈)alkenyl or (C₂-C₈)alkynyl of R^{a} is optionally substituted with one or more OH, NH₂, CO₂H, C₂-C₂₀ heterocyclyl, and wherein any aryl(C₁-C₈)alkyl, C₆-C₂₀ aryl, C₂-C₂₀ heterocyclyl, (C₃-C₇)cycloalkyl or (C₃-C₇)cycloalkyl(C₁-C₈)alkyl of R^{a} is optionally substituted with one or more —OH, —NH₂, CO₂H, C₂-C₂₀ heterocyclyl or (C₁-C₈)alkyl; each R¹¹ or R¹² can be independently H, (C₁-C₈)alkyl, C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl, (C₃-C₇)cycloalkyl(C₁-C₈)alkyl, —C(═O)R^{a} or —S(O)ₚR^{a}; or when R¹¹ and R¹² are attached to a nitrogen they may optionally can be taken together with the nitrogen to which they are both attached to form a 3 to 7 membered heterocyclic ring wherein any one carbon atom of said heterocyclic ring can optionally be replaced with —O—, —S—, —S(O)ₚ—, —NH—, —NR^{a}— or —C(O)—; R¹³ can be H or (C₁-C₈)alkyl; R¹⁴ can be H, (C₁-C₈)alkyl, NR¹¹R¹², NR¹¹C(O)R¹¹, NR¹¹C(O)OR¹¹, NR¹¹C(O)NR¹¹R¹², NR¹¹S(O)ₚR^{a}, —NR¹¹S(O)ₚ(OR¹¹) or NR¹¹SOₚNR¹¹R¹²; and wherein each (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, C₂-C₂₀ heterocyclyl(C₁-C₈)alkyl, (C₃-C₇)cycloalkyl or (C₃-C₇)cycloalkyl(C₁-C₈)alkyl of each R′, R², R^{2′}, R³, R^{3′}, R⁴, R^{4′}, R⁵, R^{5′}, R⁶, R⁷, R⁸, R^{8′} or R¹² can be independently, optionally substituted with one or more oxo, halogen, hydroxy, —NH₂, CN, N₃, —N(R^{a})₂, —NHR^{a}, —SH, —SR^{a}, —S(O)ₚR^{a}, —OR^{a}, C₁-C₈)alkyl, (C₁-C₈)haloalkyl, —C(O)R^{a}, —C(O)H, —C(═O)OR^{a}, —C(═O)OH, —C(═O)N(R^{a})₂, —C(═O)NHR^{a}, —C(═O)NH₂, —NHS(O)ₚR^{a}, —NR^{a}S(O)ₚR^{a}, —NHC(O)R^{a}, —NR^{a}C(O)R^{a}, —NHC(O)OR^{a}, —NR^{a}C(O)OR^{a}, —NR^{a}C(O)NHR^{a}, —NR^{a}C(O)N(R^{a})₂, —NR^{a}C(O)NH₂, —NHC(O)NHR^{a}, —NHC(O)N(R^{a})₂, —NHC(O)NH₂, ═NH, ═NOH, ═NOR^{a}, —NR^{a}S(O)ₚNHR^{a}, —NR^{a}S(O)ₚN(R^{a})₂, NR^{a}S(O)ₚNH₂, —NHS(O)ₚNHR^{a}, —NHS(O)ₚN(R^{a})₂, —NHS(O)ₚNH₂, —OC(═O)R^{a}, —OP(O)(OH)₂ or R^{a}.

Examples of Compounds of Formula (B18) include:

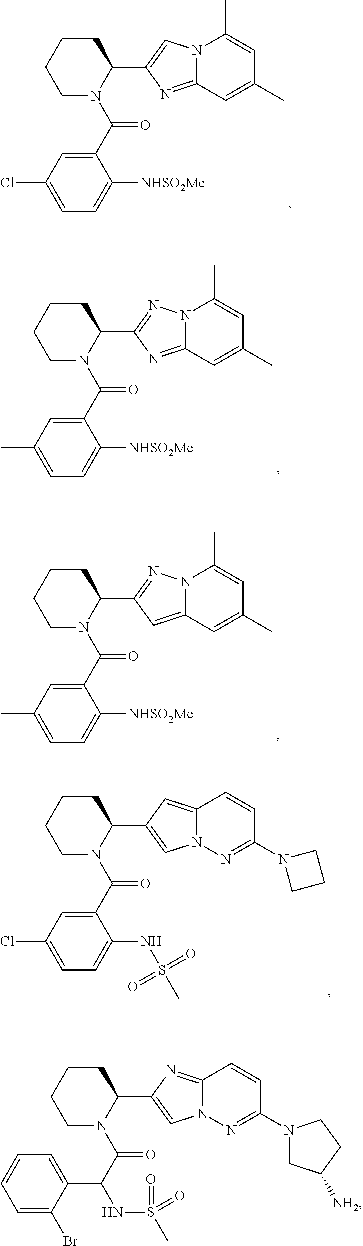

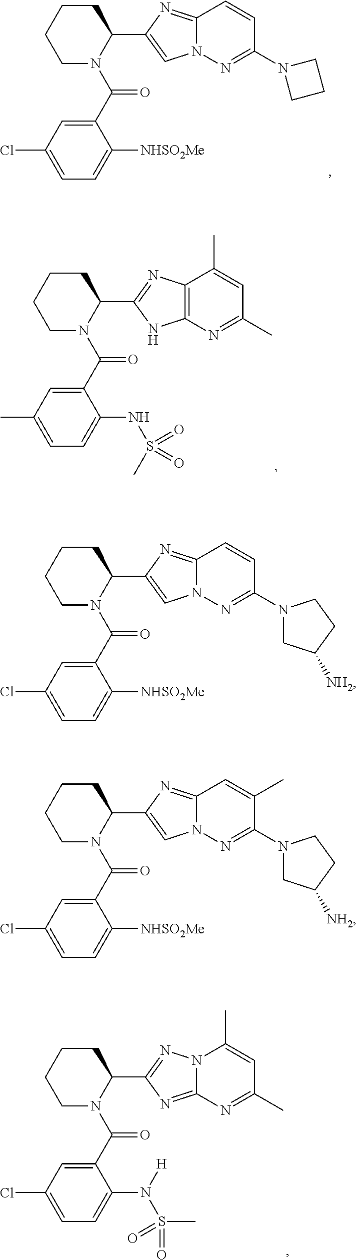

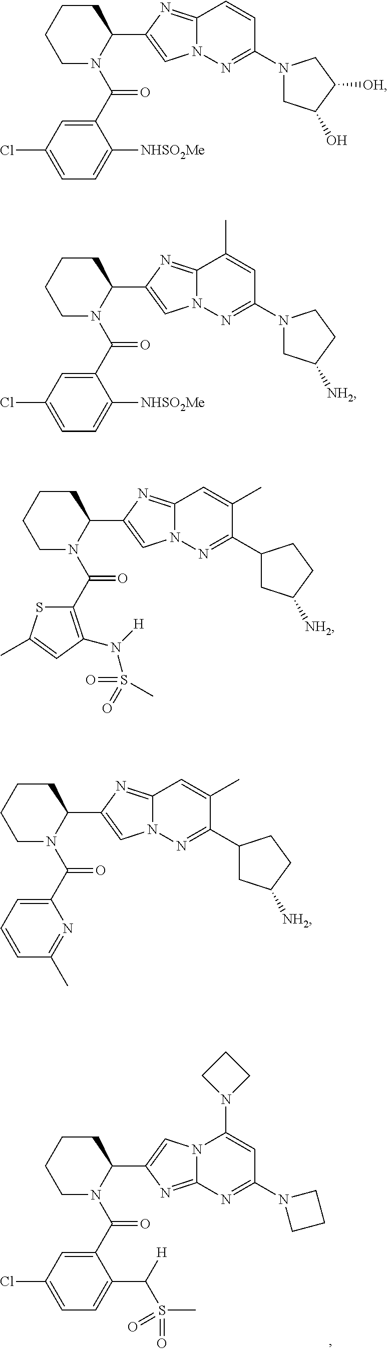

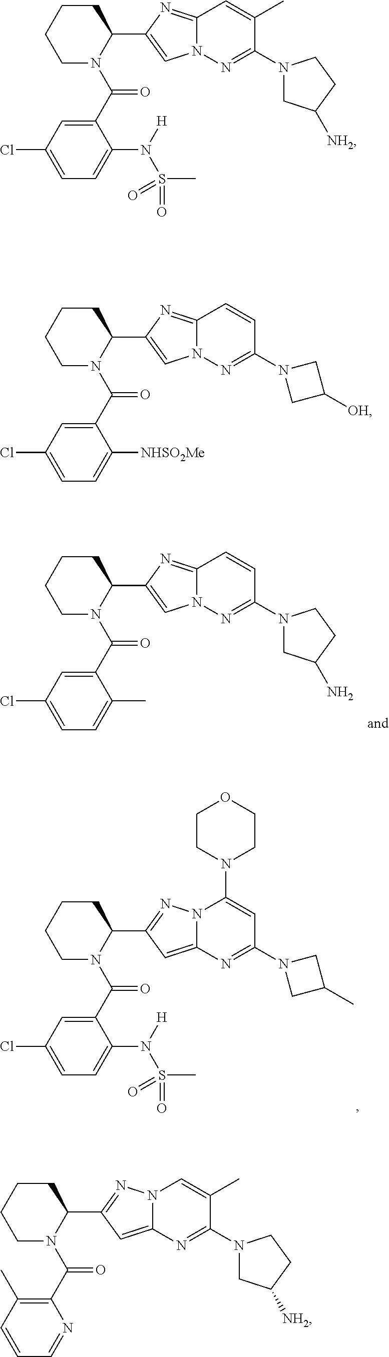

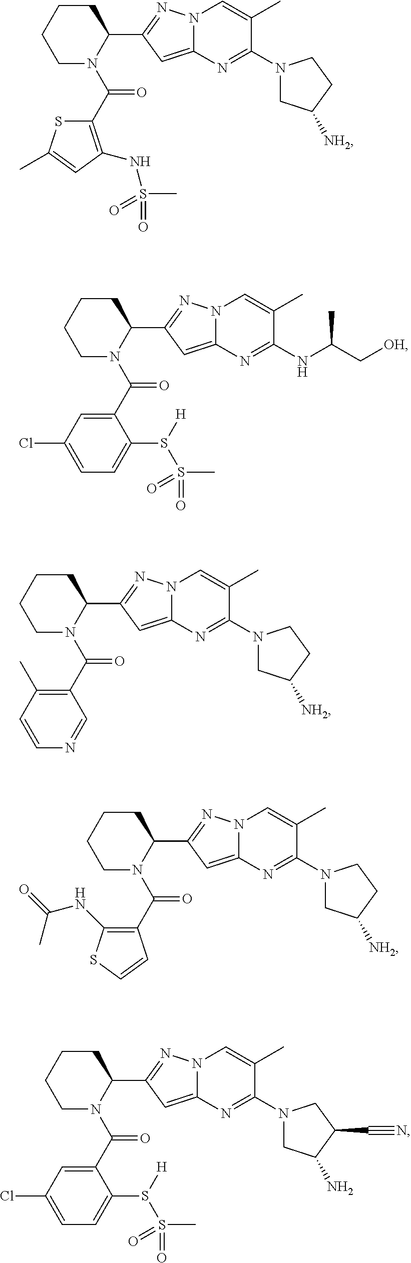

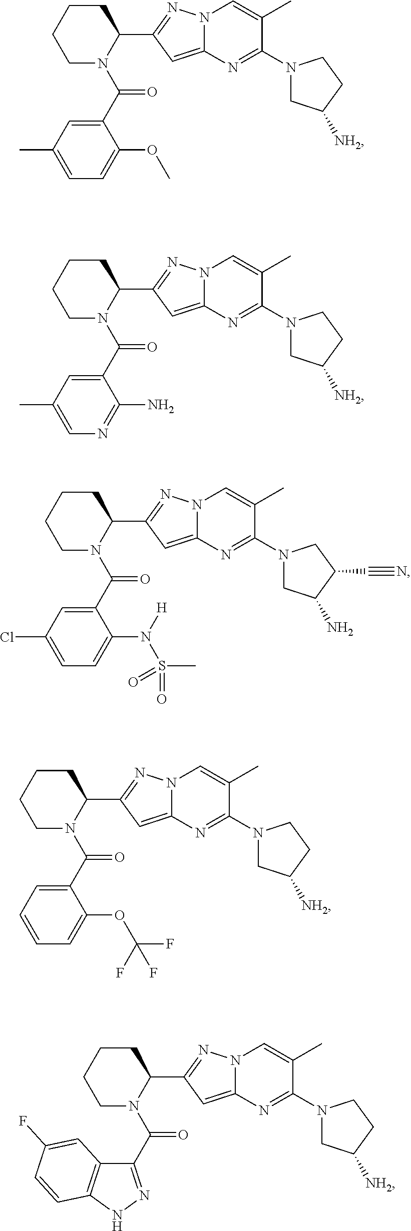 [SMILES: COc1ccc(C)cc1C(=O)N1CCCC[C@H]1c1cc2nc(N3CC[C@H](N)C3)c(C)cn2n1 ; Cc1cnc(N)c(C(=O)N2CCCC[C@H]2c2cc3nc(N4CC[C@H](N)C4)c(C)cn3n2)c1 ; Cc1cn2nc([C@@H]3CCCCN3C(=O)c3cc(Cl)ccc3NS(C)(=O)=O)cc2nc1N1C[C@@H](N)[C@@H](C#N)C1 ; Cc1cn2nc([C@@H]3CCCCN3C(=O)c3ccccc3OC(F)(F)F)cc2nc1N1CC[C@H](N)C1 ; Cc1cn2nc([C@@H]3CCCCN3C(=O)c3n[nH]c4ccc(F)cc34)cc2nc1N1CC[C@H](N)C1]

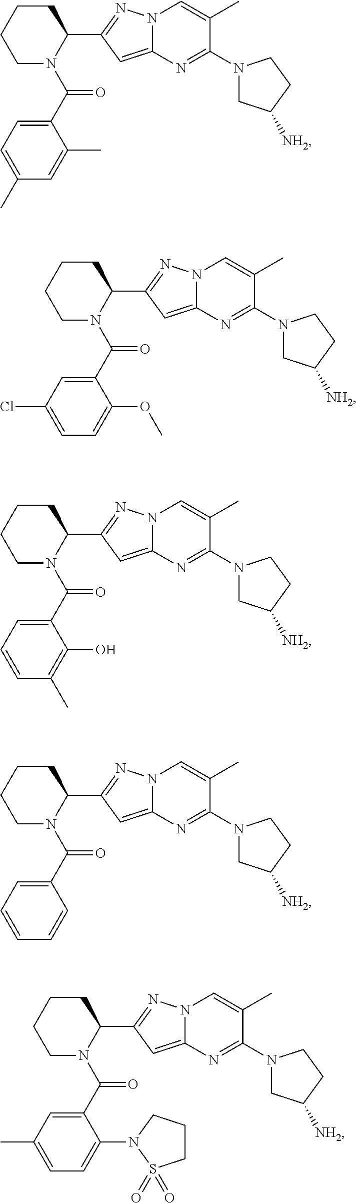 [SMILES: Cc1ccc(C(=O)N2CCCC[C@H]2c2cc3nc(N4CC[C@H](N)C4)c(C)cn3n2)c(C)c1 ; COc1ccc(Cl)cc1C(=O)N1CCCC[C@H]1c1cc2nc(N3CC[C@H](N)C3)c(C)cn2n1 ; Cc1cn2nc([C@@H]3CCCCN3C(=O)c3cccc(C)c3O)cc2nc1N1CC[C@H](N)C1 ; Cc1cn2nc([C@@H]3CCCCN3C(=O)c3ccccc3)cc2nc1N1CC[C@H](N)C1 ; Cc1ccc(N2CCCS2(=O)=O)c(C(=O)N2CCCC[C@H]2c2cc3nc(N4CC[C@H](N)C4)c(C)cn3n2)c1]

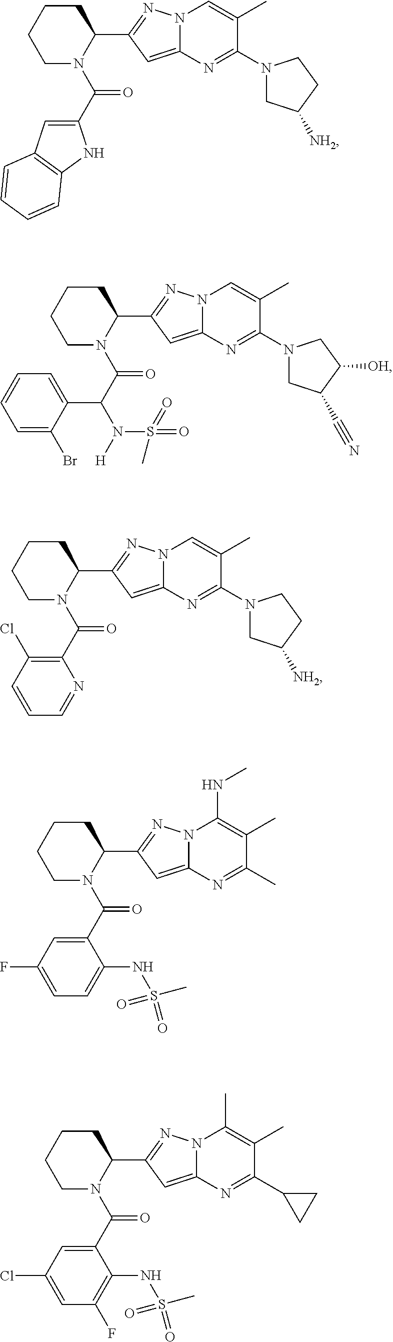 [SMILES: Cc1cn2nc([C@@H]3CCCCN3C(=O)c3cc4ccccc4[nH]3)cc2nc1N1CC[C@H](N)C1 ; Cc1cn2nc([C@@H]3CCCCN3C(=O)C(NS(C)(=O)=O)c3ccccc3Br)cc2nc1N1C[C@@H](C#N)[C@@H](O)C1 ; Cc1cn2nc([C@@H]3CCCCN3C(=O)c3ncccc3Cl)cc2nc1N1CC[C@H](N)C1 ; CNc1c(C)c(C)nc2cc([C@@H]3CCCCN3C(=O)c3cc(F)ccc3NS(C)(=O)=O)nn12 ; Cc1c(C2CC2)nc2cc([C@@H]3CCCCN3C(=O)c3cc(Cl)cc(F)c3NS(C)(=O)=O)nn2c1C]

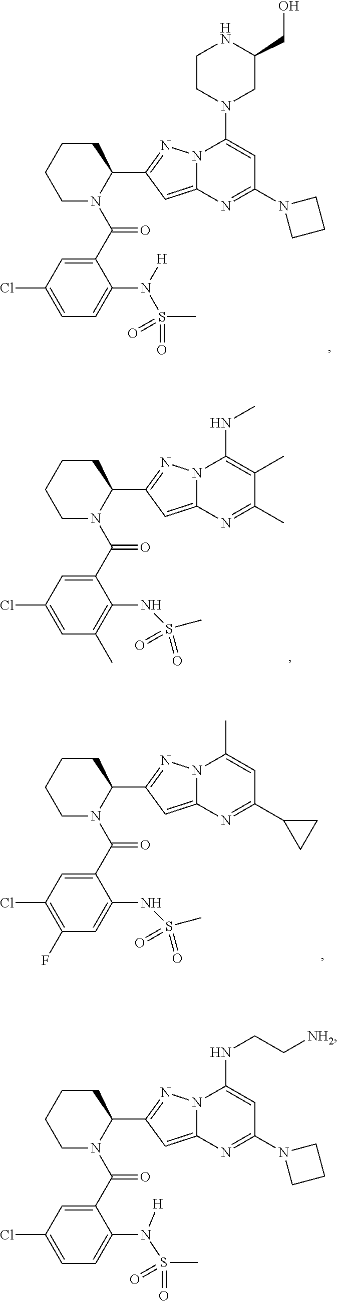 [SMILES: CS(=O)(=O)Nc1ccc(Cl)cc1C(=O)N1CCCC[C@H]1c1cc2nc(N3CCC3)cc(N3CCN[C@@H](CO)C3)n2n1 ; CNc1c(C)c(C)nc2cc([C@@H]3CCCCN3C(=O)c3cc(Cl)cc(C)c3NS(C)(=O)=O)nn12 ; Cc1cc(C2CC2)nc2cc([C@@H]3CCCCN3C(=O)c3cc(Cl)c(F)cc3NS(C)(=O)=O)nn12 ; CS(=O)(=O)Nc1ccc(Cl)cc1C(=O)N1CCCC[C@H]1c1cc2nc(N3CCC3)cc(NCCN)n2n1]

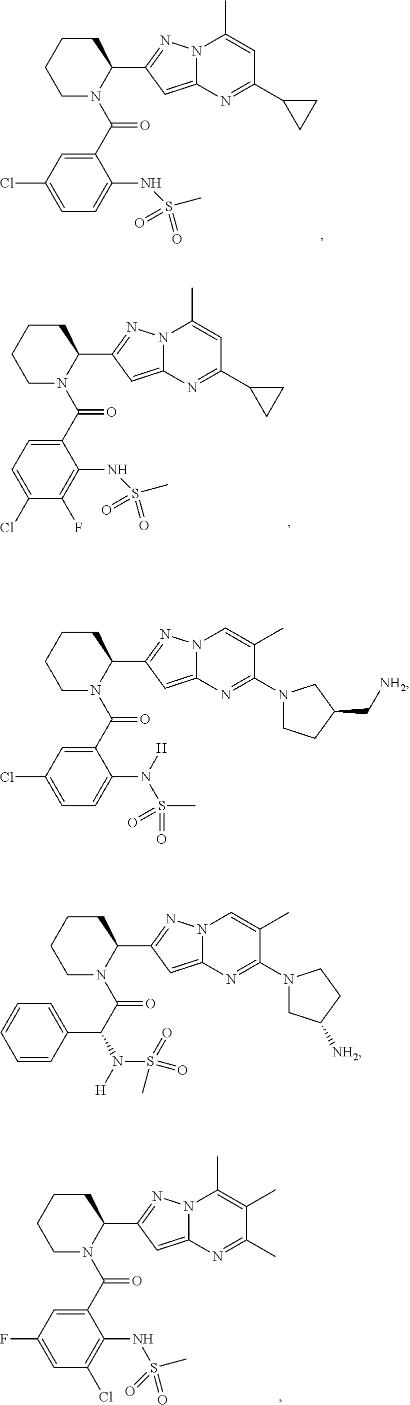 [SMILES: Cc1cc(C2CC2)nc2cc([C@@H]3CCCCN3C(=O)c3cc(Cl)ccc3NS(C)(=O)=O)nn12 ; Cc1cc(C2CC2)nc2cc([C@@H]3CCCCN3C(=O)c3ccc(Cl)c(F)c3NS(C)(=O)=O)nn12 ; Cc1cn2nc([C@@H]3CCCCN3C(=O)c3cc(Cl)ccc3NS(C)(=O)=O)cc2nc1N1CC[C@H](CN)C1 ; Cc1cn2nc([C@@H]3CCCCN3C(=O)[C@H](NS(C)(=O)=O)c3ccccc3)cc2nc1N1CC[C@H](N)C1 ; Cc1nc2cc([C@@H]3CCCCN3C(=O)c3cc(F)cc(Cl)c3NS(C)(=O)=O)nn2c(C)c1C]

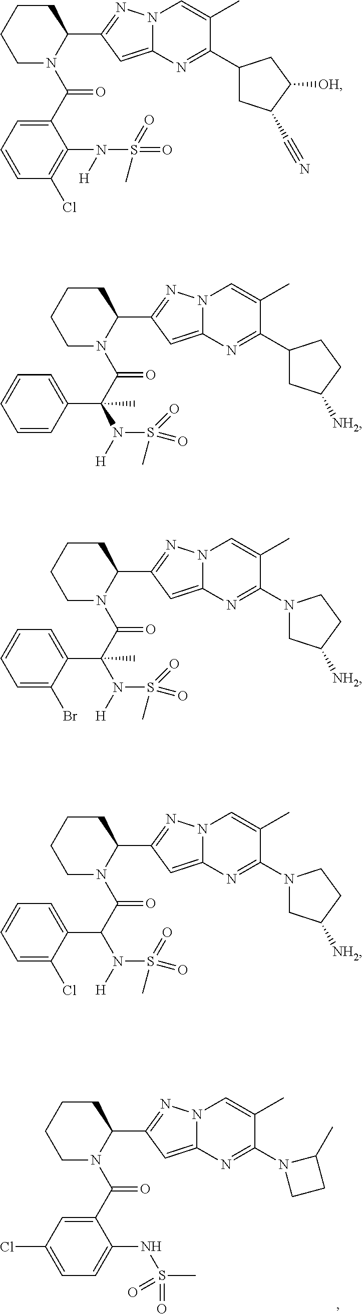 [SMILES: Cc1cn2nc([C@@H]3CCCCN3C(=O)c3cccc(Cl)c3NS(C)(=O)=O)cc2nc1C1C[C@@H](C#N)[C@@H](O)C1 ; Cc1cn2nc([C@@H]3CCCCN3C(=O)[C@@](C)(NS(C)(=O)=O)c3ccccc3)cc2nc1C1CC[C@H](N)C1 ; Cc1cn2nc([C@@H]3CCCCN3C(=O)[C@@](C)(NS(C)(=O)=O)c3ccccc3Br)cc2nc1N1CC[C@H](N)C1 ; Cc1cn2nc([C@@H]3CCCCN3C(=O)C(NS(C)(=O)=O)c3ccccc3Cl)cc2nc1N1CC[C@H](N)C1 ; Cc1cn2nc([C@@H]3CCCCN3C(=O)c3cc(Cl)ccc3NS(C)(=O)=O)cc2nc1N1CCC1C]

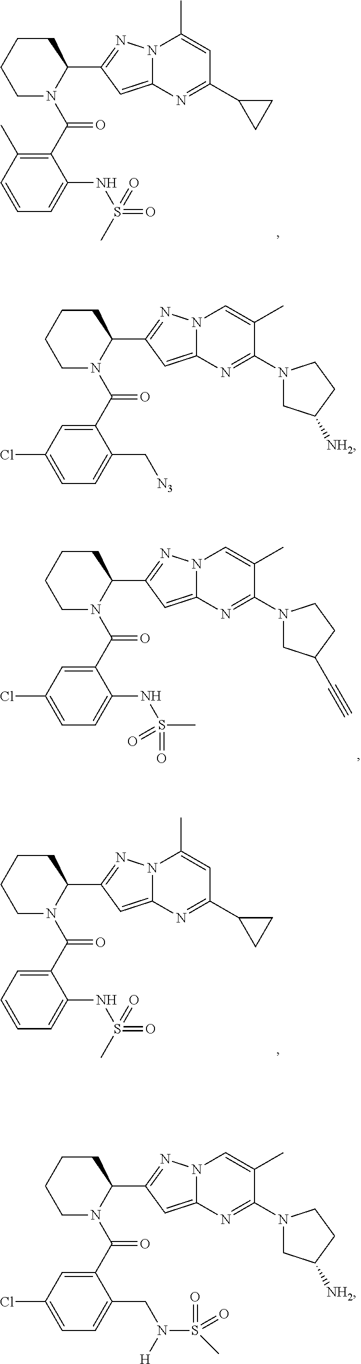 [SMILES: Cc1cccc(NS(C)(=O)=O)c1C(=O)N1CCCC[C@H]1c1cc2nc(C3CC3)cc(C)n2n1 ; Cc1cn2nc([C@@H]3CCCCN3C(=O)c3cc(Cl)ccc3CN=[N+]=[N-])cc2nc1N1CC[C@H](N)C1 ; C#CC1CCN(c2nc3cc([C@@H]4CCCCN4C(=O)c4cc(Cl)ccc4NS(C)(=O)=O)nn3cc2C)C1 ; Cc1cc(C2CC2)nc2cc([C@@H]3CCCCN3C(=O)c3ccccc3NS(C)(=O)=O)nn12 ; Cc1cn2nc([C@@H]3CCCCN3C(=O)c3cc(Cl)ccc3CNS(C)(=O)=O)cc2nc1N1CC[C@H](N)C1]

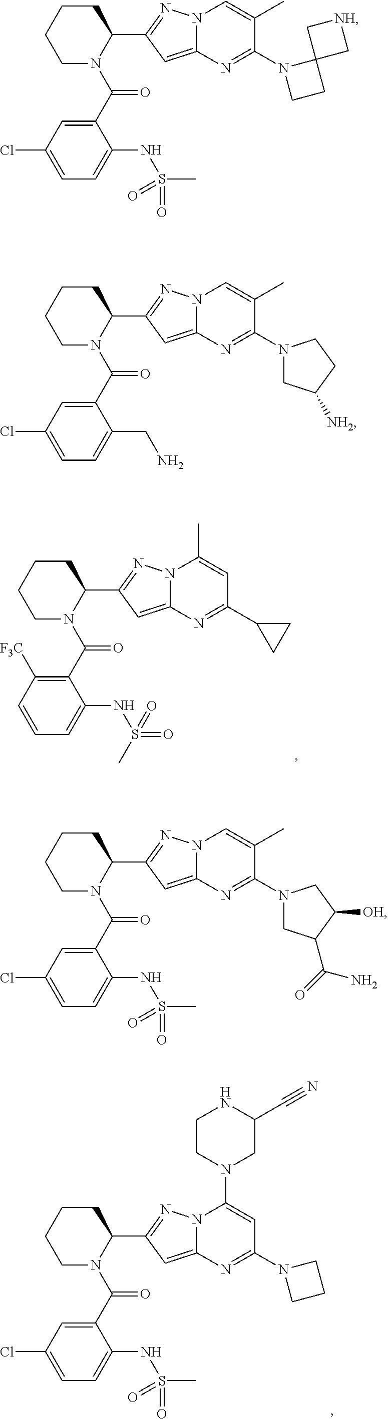 [SMILES: Cc1cn2nc([C@@H]3CCCCN3C(=O)c3cc(Cl)ccc3NS(C)(=O)=O)cc2nc1N1CCC12CNC2 ; Cc1cn2nc([C@@H]3CCCCN3C(=O)c3cc(Cl)ccc3CN)cc2nc1N1CC[C@H](N)C1 ; Cc1cc(C2CC2)nc2cc([C@@H]3CCCCN3C(=O)c3c(NS(C)(=O)=O)cccc3C(F)(F)F)nn12 ; Cc1cn2nc([C@@H]3CCCCN3C(=O)c3cc(Cl)ccc3NS(C)(=O)=O)cc2nc1N1CC(C(N)=O)[C@H](O)C1 ; CS(=O)(=O)Nc1ccc(Cl)cc1C(=O)N1CCCC[C@H]1c1cc2nc(N3CCC3)cc(N3CCNC(C#N)C3)n2n1]

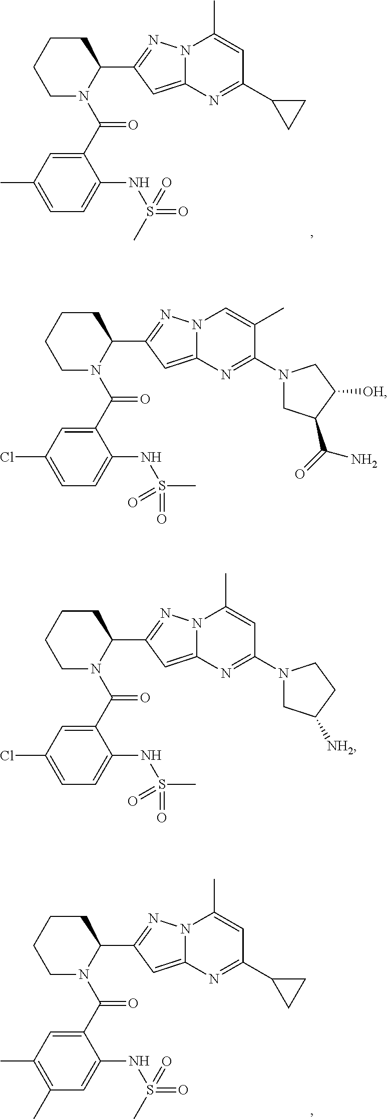 [SMILES: Cc1ccc(NS(C)(=O)=O)c(C(=O)N2CCCC[C@H]2c2cc3nc(C4CC4)cc(C)n3n2)c1 ; Cc1cn2nc([C@@H]3CCCCN3C(=O)c3cc(Cl)ccc3NS(C)(=O)=O)cc2nc1N1C[C@H](C(N)=O)[C@@H](O)C1 ; Cc1cc(N2CC[C@H](N)C2)nc2cc([C@@H]3CCCCN3C(=O)c3cc(Cl)ccc3NS(C)(=O)=O)nn12 ; Cc1cc(NS(C)(=O)=O)c(C(=O)N2CCCC[C@H]2c2cc3nc(C4CC4)cc(C)n3n2)cc1C]

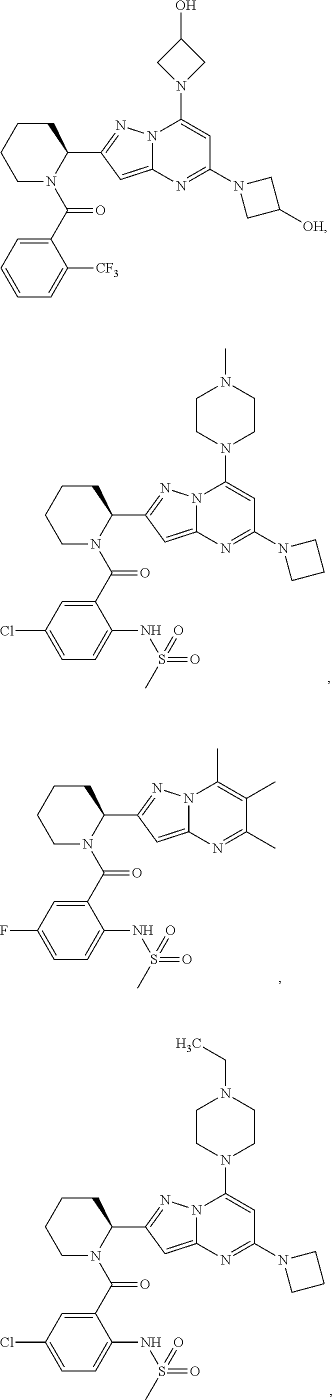 [SMILES: O=C(c1ccccc1C(F)(F)F)N1CCCC[C@H]1c1cc2nc(N3CC(O)C3)cc(N3CC(O)C3)n2n1 ; CN1CCN(c2cc(N3CCC3)nc3cc([C@@H]4CCCCN4C(=O)c4cc(Cl)ccc4NS(C)(=O)=O)nn23)CC1 ; Cc1nc2cc([C@@H]3CCCCN3C(=O)c3cc(F)ccc3NS(C)(=O)=O)nn2c(C)c1C ; CCN1CCN(c2cc(N3CCC3)nc3cc([C@@H]4CCCCN4C(=O)c4cc(Cl)ccc4NS(C)(=O)=O)nn23)CC1]

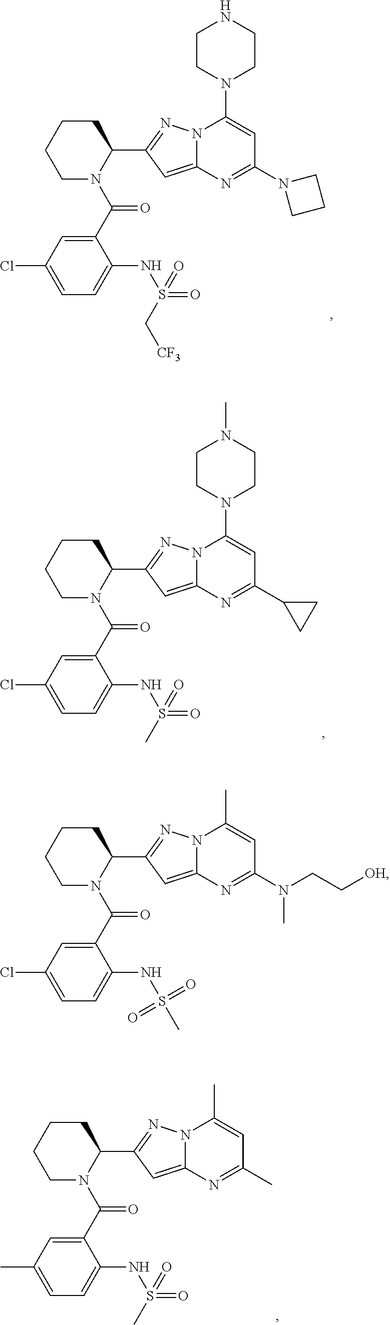

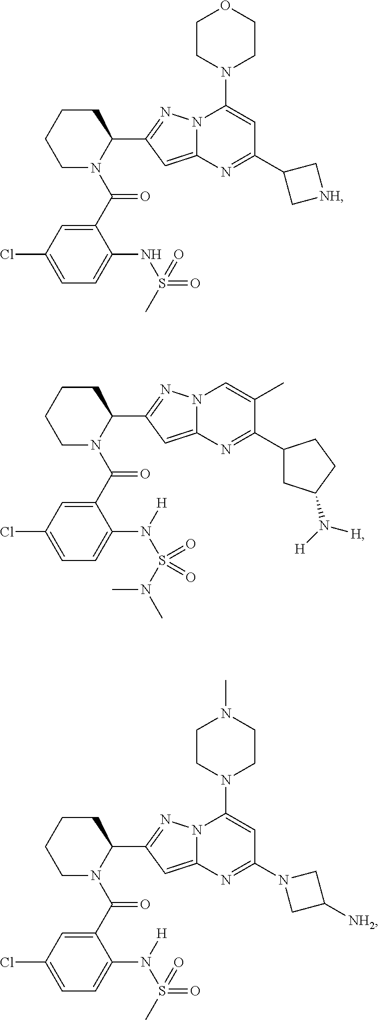

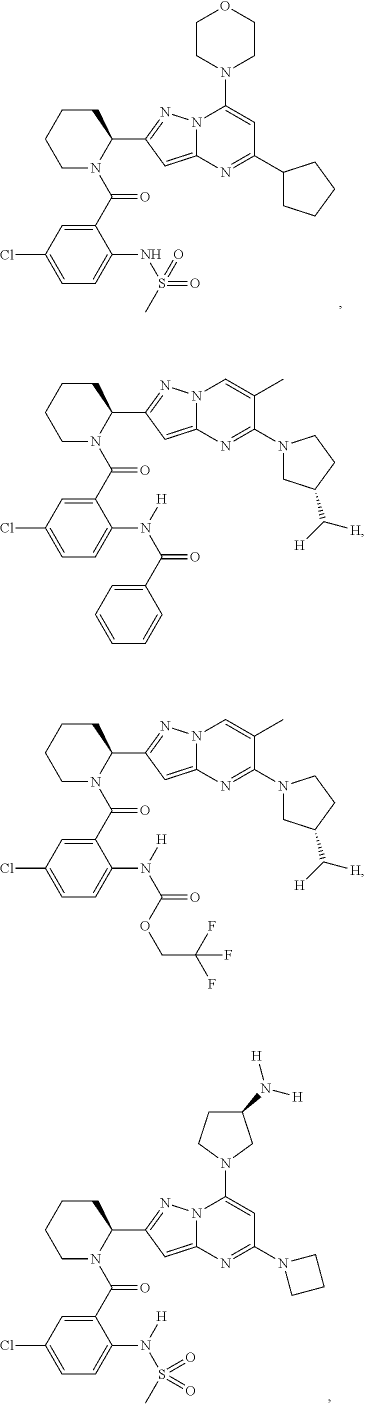

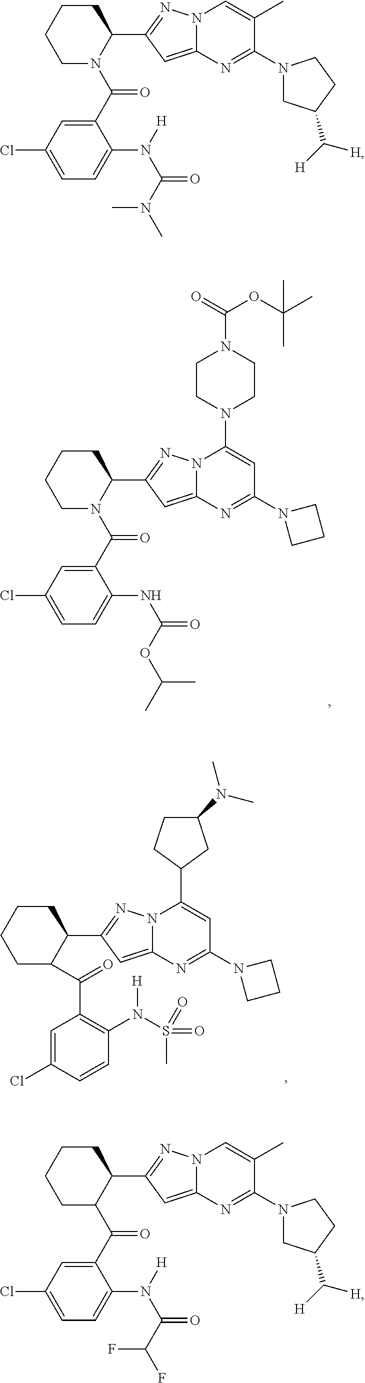

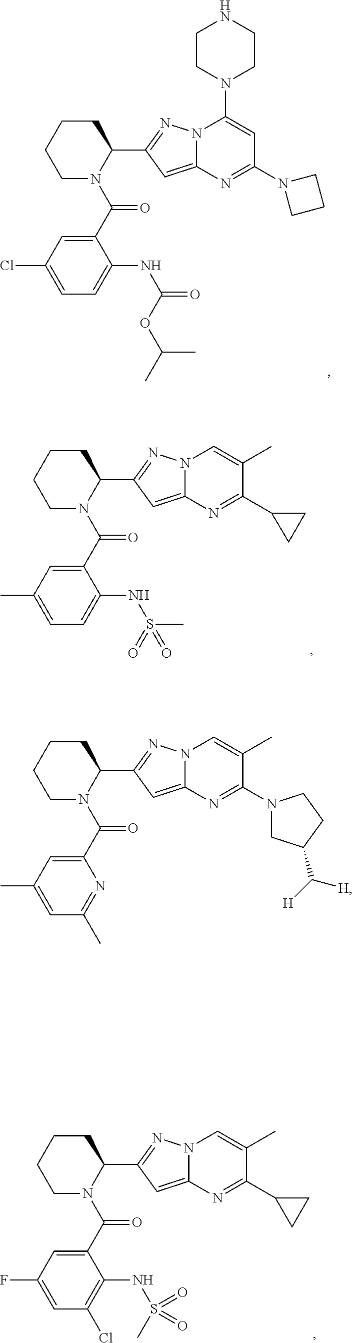

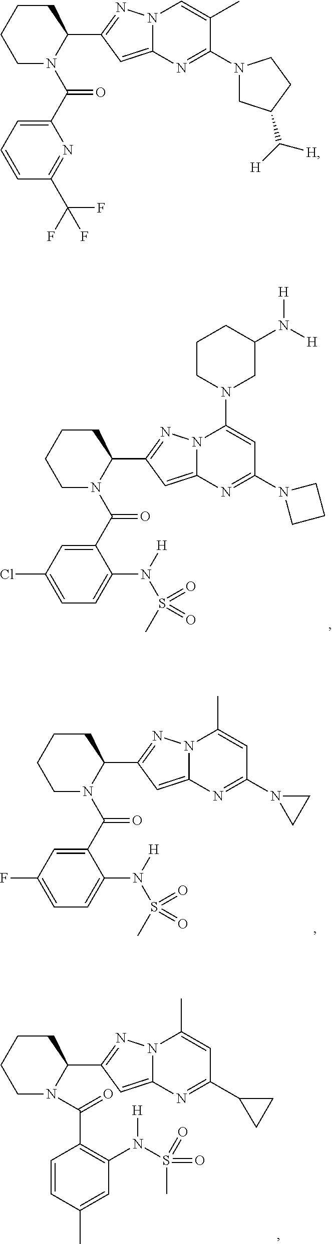

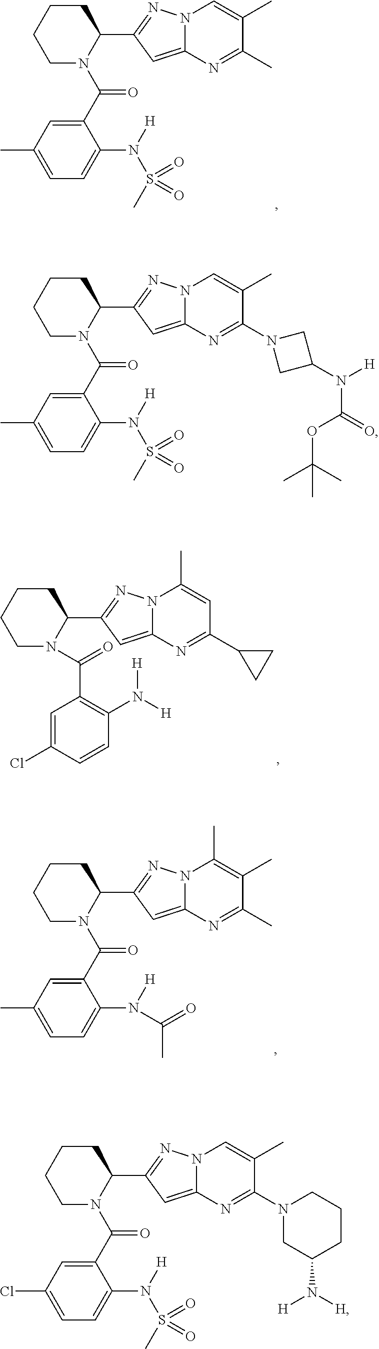

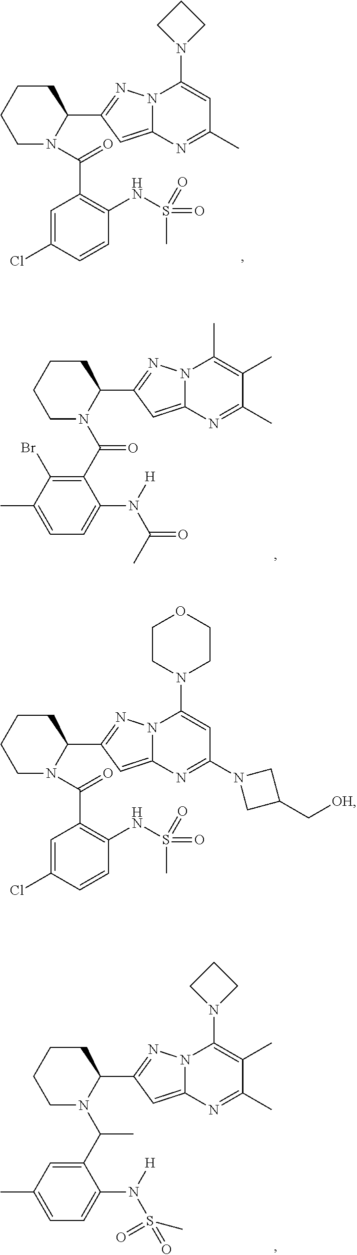

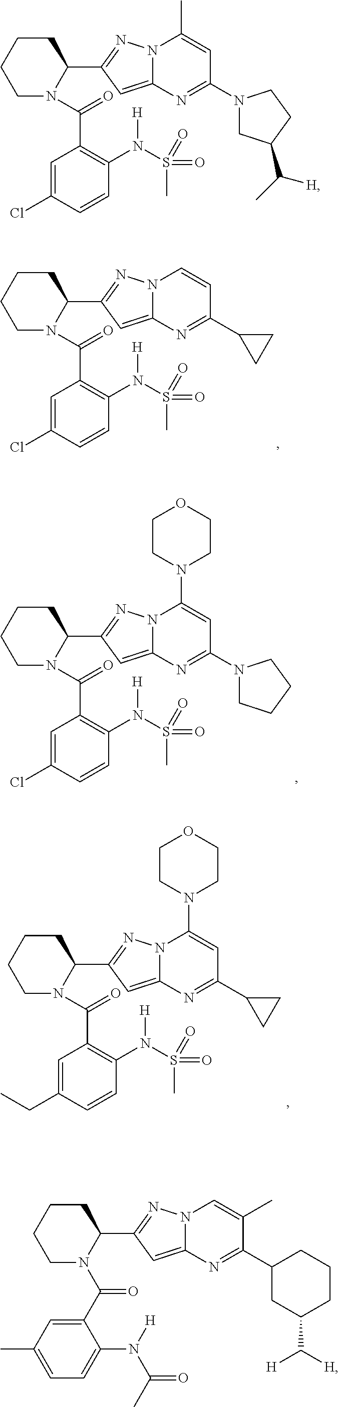

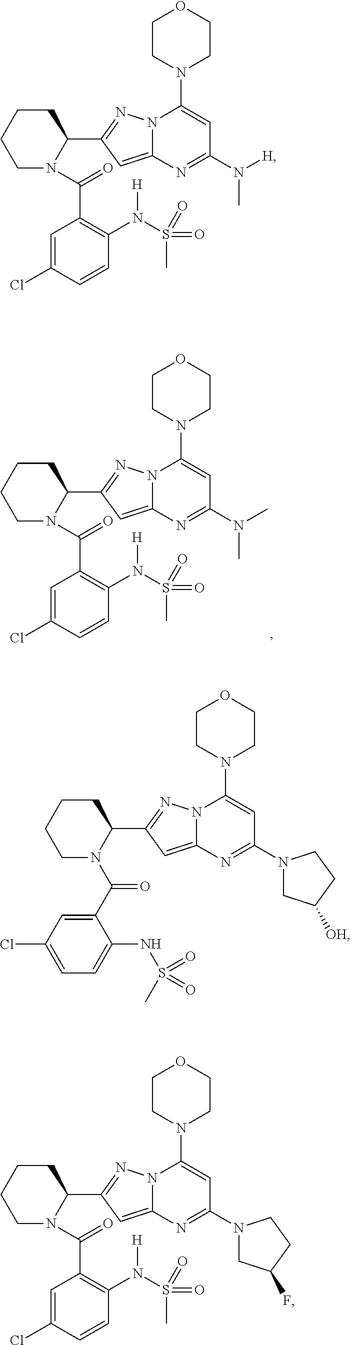

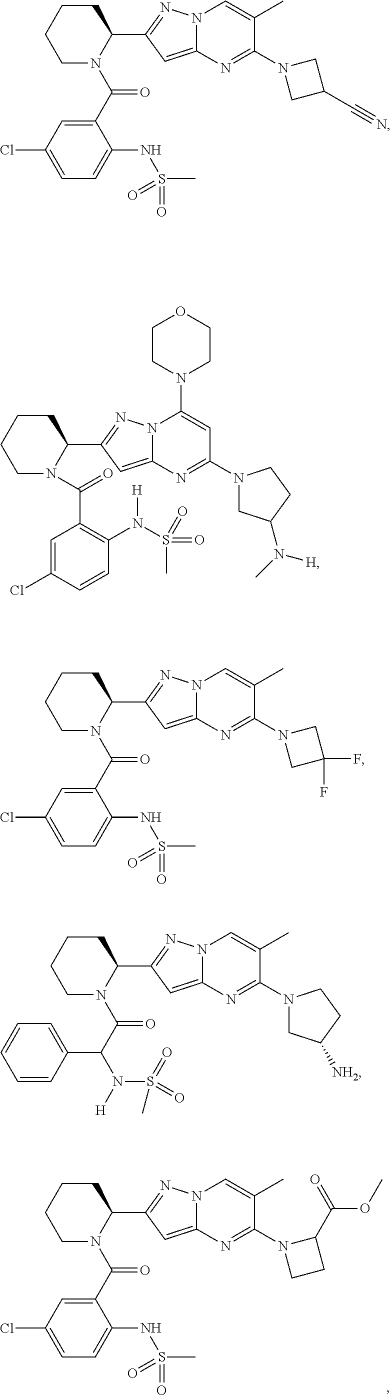

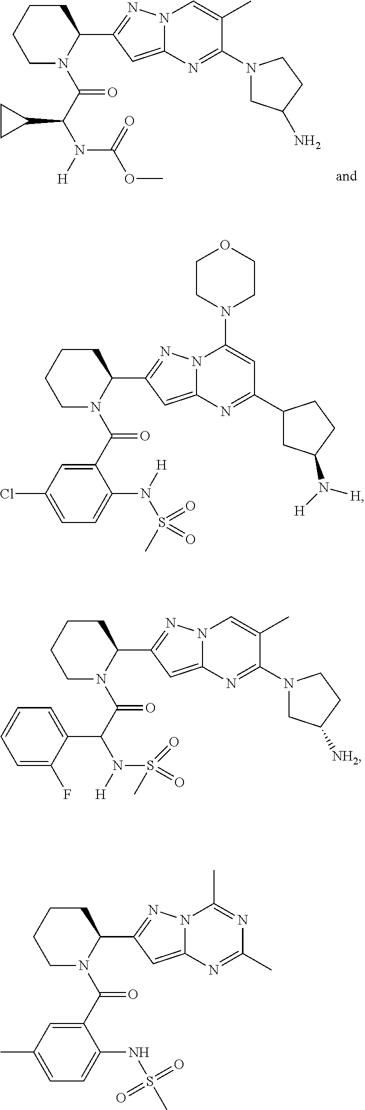

### Compounds of Formula (B19)

Compounds of the general Formula (B19) are described in U.S. Publication No. 2013/0164280, published Jun. 27, 2013, which is hereby incorporated by reference in its entirety. Formula (B19) has the structure:

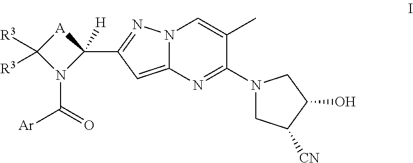

or a salt or ester thereof, wherein: A can be —(C(R⁴)₂)ₙ— wherein any one C(R⁴)₂ of said —(C(R⁴)₂)ₙ— may be optionally replaced with —O—, —S—, —S(O)_{P}—, NH or NR^{a}; n can be 3,4, 5 or 6; each p can be 1 or 2; Ar can be a C₂-C₂₀ heterocyclyl group or a C₆-C₂₀ aryl group, wherein the C₂-C₂₀ heterocyclyl group or the C₆-C₂₀ aryl group is optionally substituted with 1, 2, 3, 4 or 5 R⁶; each R³, R⁴ or R⁶ can be independently H, oxo, OR¹¹, NR¹¹R¹², NR¹¹C(O)R¹¹, NR¹¹C(O)OR¹¹, NR¹¹C(O)NR¹¹R¹², N₃, CN, NO₂, SR¹¹, S(O)ₚR^{a}, NR¹¹S(O)ₚR^{a}, —C(═O)R¹¹, —C(═O)OR¹¹, —C(═O)NR¹¹R¹², —C(═O)SR¹¹, —S(O)ₚ(OR¹¹), —SO₂NR¹¹R¹², —NR¹¹S(O)ₚ(OR¹¹), —NR¹¹SOₚNR¹¹R¹², NR¹¹C(═NR¹¹)NR¹¹R¹², halogen, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl; or two R⁴ on adjacent carbon atoms, when taken together, may optionally form a double bond between the two carbons to which they are attached or may form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; or four R⁴ on adjacent carbon atoms, when taken together, may optionally form an optionally substituted C₆ aryl ring; or two R⁴ on the same carbon atom, when taken together, may optionally form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; or two R⁶ on adjacent carbon atoms, when taken together, may optionally form a (C₃-C₇)cycloalkyl ring wherein one carbon atom of said (C₃-C₇)cycloalkyl ring may be optionally replaced by —O—, —S—, —S(O)_{P}—, —NH— or —NR^{a}—; each R^{a} can be independently (C₁-C₈)alkyl, (C₁-C₈)haloalkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl wherein any (C₁-C₈)alkyl, (C₁-C₈)haloalkyl, (C₂-C₈)alkenyl or (C₂-C₈)alkynyl of R^{a} is optionally substituted with one or more OH, NH₂, CO₂H, C₂-C₂₀ heterocyclyl, and wherein any aryl(C₁-C₈)alkyl, C₆-C₂₀ aryl, C₂-C₂₀ heterocyclyl, (C₃-C₇)cycloalkyl or (C₄-C₈)carbocyclylalkyl of R^{a} is optionally substituted with one or more OH, NH₂, CO₂H, C₂-C₂₀ heterocyclyl or (C₁-C₈)alkyl; each R¹¹ or R¹² can be independently H, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl, C₄-C₈)carbocyclylalkyl, C(═O)R^{a}, —S(O)ₚR^{a} or aryl(C₁-C₈)alkyl; or R¹¹ and R¹² can be taken together with a nitrogen to which they are both attached form a 3 to 7 membered heterocyclic ring wherein any one carbon atom of said heterocyclic ring can optionally be replaced with —O—, —S—, —S(O)ₚ—, —NH—, —NR^{a}—; or —C(O)—; and wherein each (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl of each R⁶, R¹¹ or R¹² can be, independently, optionally substituted with one or more oxo, halogen, hydroxy, NH₂, CN, N₃, N(R^{a})₂, NHR^{a}, SH, SR^{a}, S(O)ₚR^{a}, OR^{a}, (C₁-C₈)alkyl, (C₁-C₈)haloalkyl, —C(O)R^{a}, —C(O)H, —C(═O)OR^{a}, —C(═O)OH, —C(═O)N(R^{a})₂, —C(═O)NHR^{a}, —C(═O)NH₂, NHS(O)ₚR^{a}, NR^{a}S(O)ₚR^{a}, NHC(O)R^{a}, NR^{a}C(O)R^{a}, NHC(O)OR^{a}, NR^{a}C(O)OR^{a}, NR^{a}C(O)NHR^{a}, NR^{a}C(O)N(R^{a})₂, NR^{a}C(O)NH₂, NHC(O)NHR^{a}, NHC(O)N(R^{a})₂, NHC(O)NH₂, ═NH, ═NOH, ═NOR^{a}, NR^{a}S(O)ₚNHR^{a}, NR^{a}S(O)ₚN(R^{a})₂, NR^{a}S(O)ₚNH₂, NHS(O)ₚNHR^{a}, NHS(O)ₚN(Ra)₂, NHS(O)ₚNH₂, —OC(═O)R^{a}, —OP(O)(OH)₂ or R^{a}.

Examples of Compounds of Formula (B19) include:

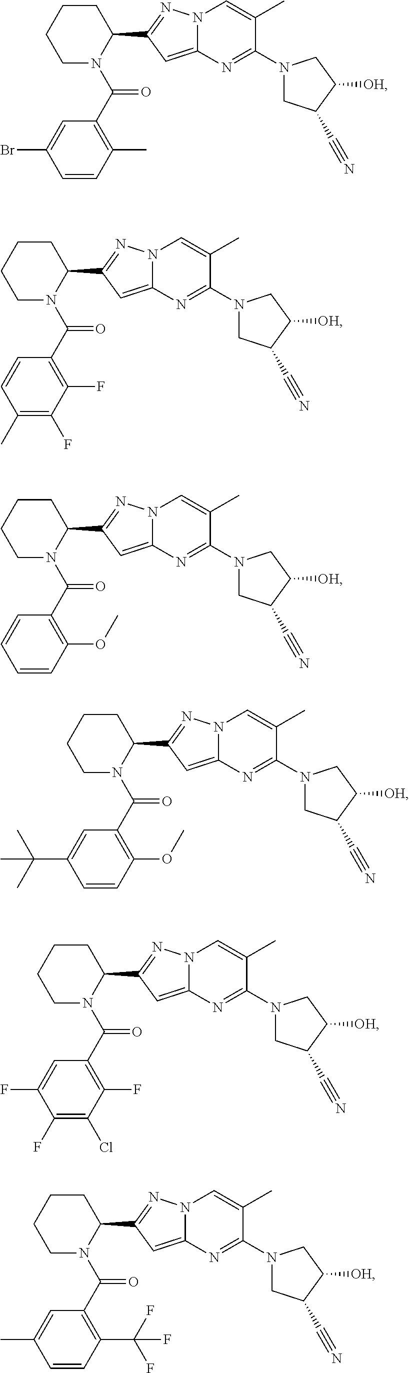 [SMILES: Cc1ccc(Br)cc1C(=O)N1CCCC[C@H]1c1cc2nc(N3C[C@@H](C#N)[C@@H](O)C3)c(C)cn2n1 ; Cc1cn2nc([C@@H]3CCCCN3C(=O)c3ccc(C)c(F)c3F)cc2nc1N1C[C@@H](C#N)[C@@H](O)C1 ; COc1ccccc1C(=O)N1CCCC[C@H]1c1cc2nc(N3C[C@@H](C#N)[C@@H](O)C3)c(C)cn2n1 ; COc1ccc(C(C)(C)C)cc1C(=O)N1CCCC[C@H]1c1cc2nc(N3C[C@@H](C#N)[C@@H](O)C3)c(C)cn2n1 ; Cc1cn2nc([C@@H]3CCCCN3C(=O)c3cc(F)c(F)c(Cl)c3F)cc2nc1N1C[C@@H](C#N)[C@@H](O)C1 ; Cc1ccc(C(F)(F)F)c(C(=O)N2CCCC[C@H]2c2cc3nc(N4C[C@@H](C#N)[C@@H](O)C4)c(C)cn3n2)c1]

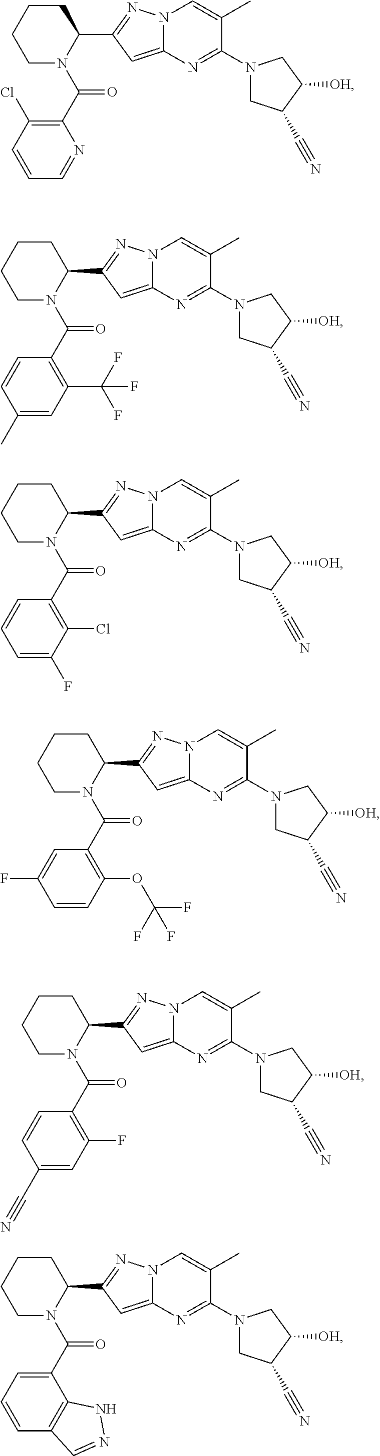

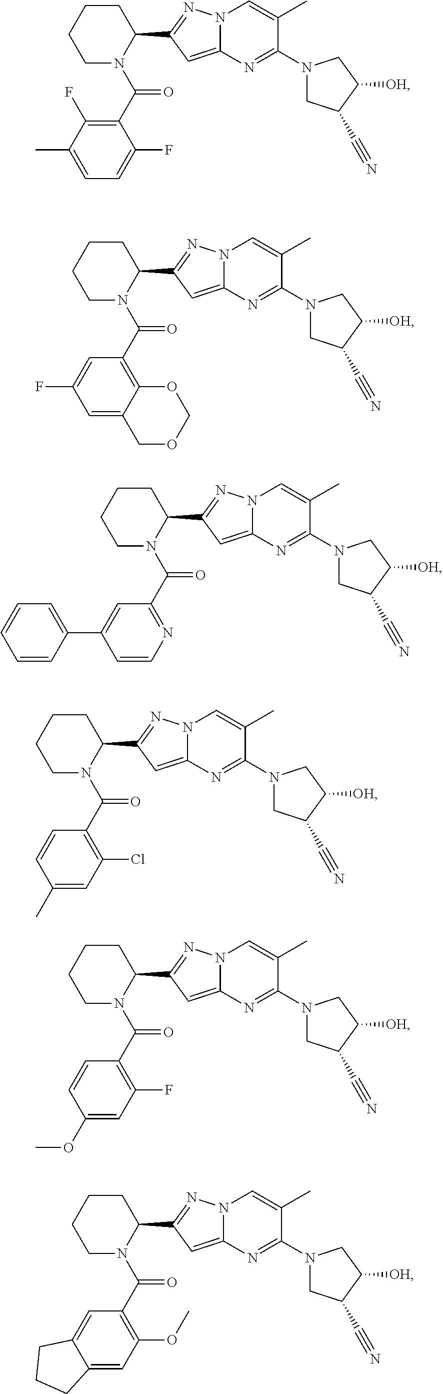

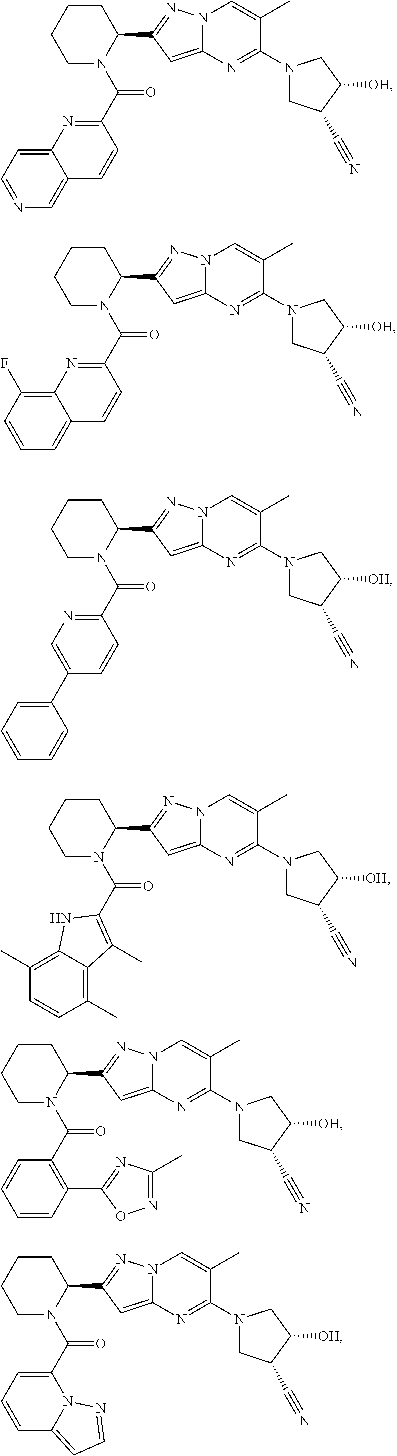

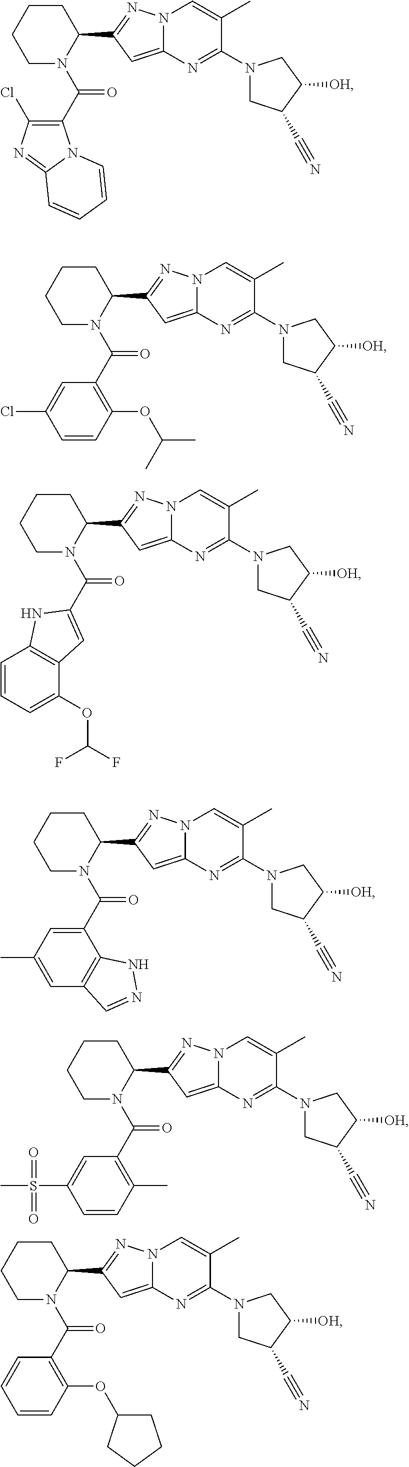

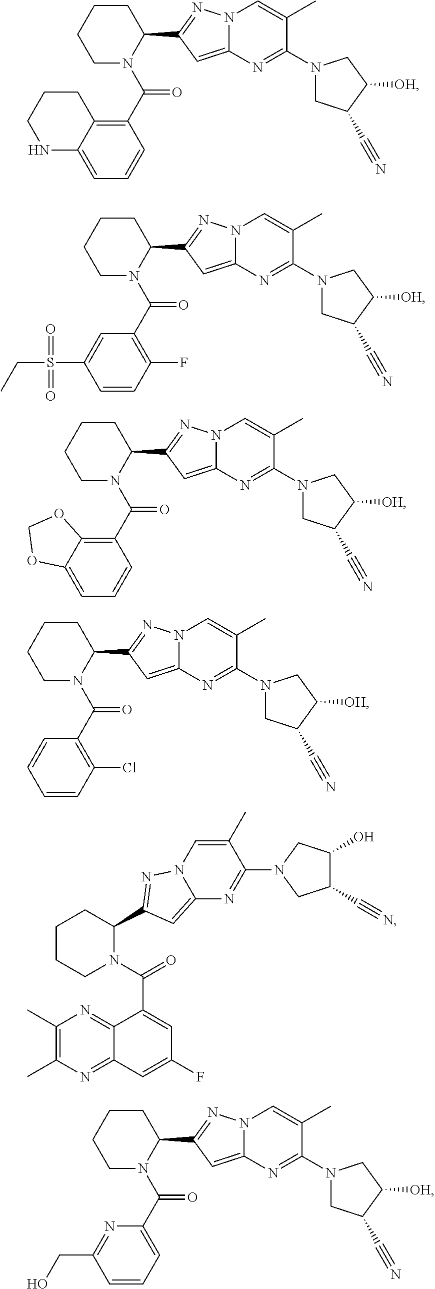

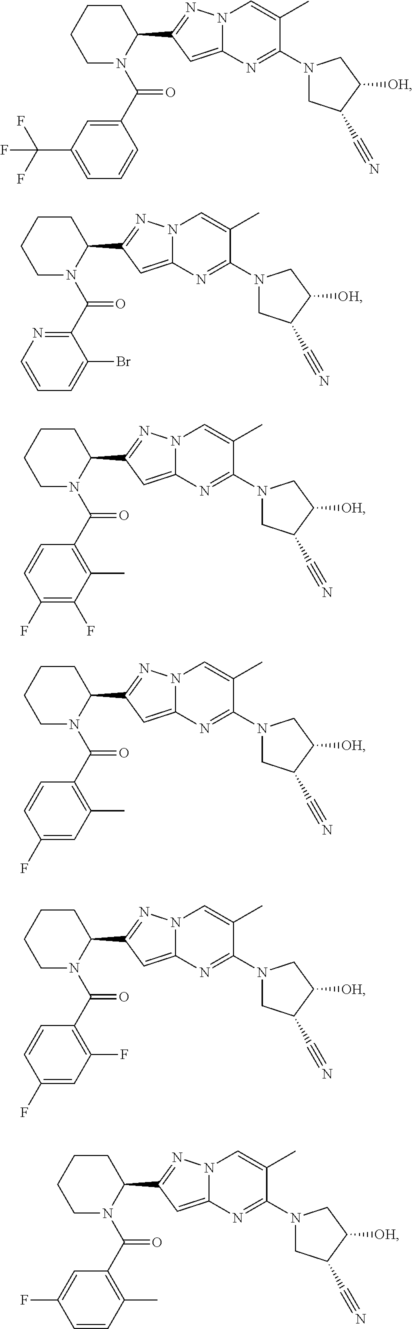

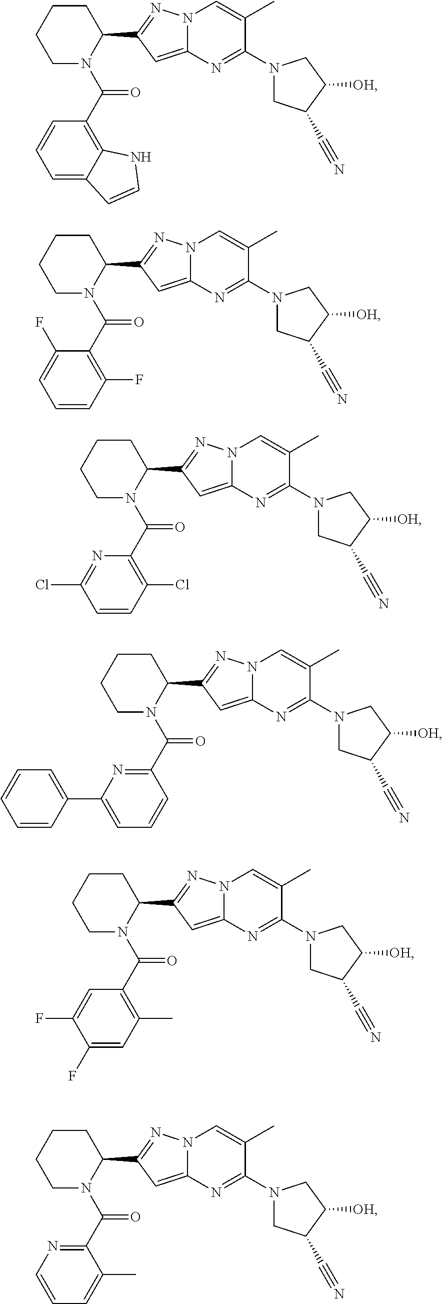

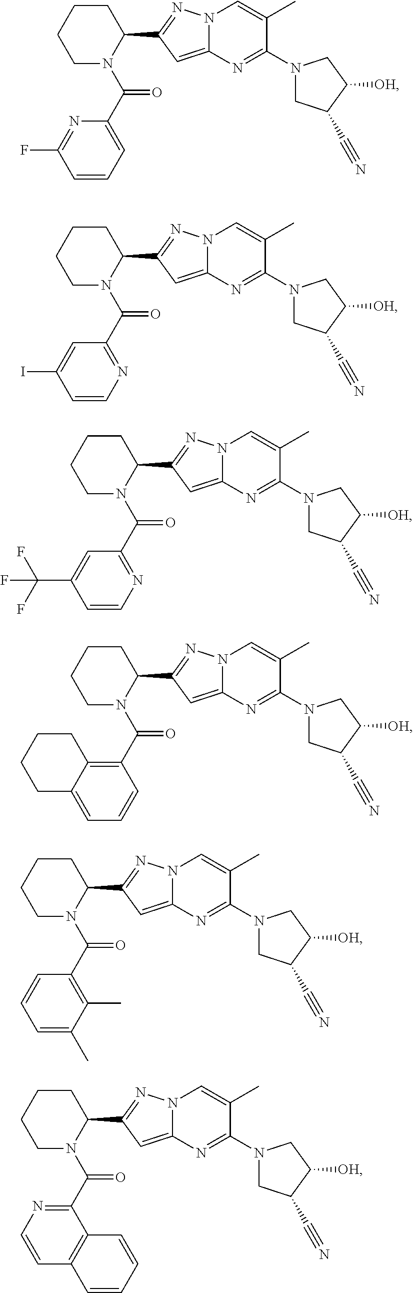

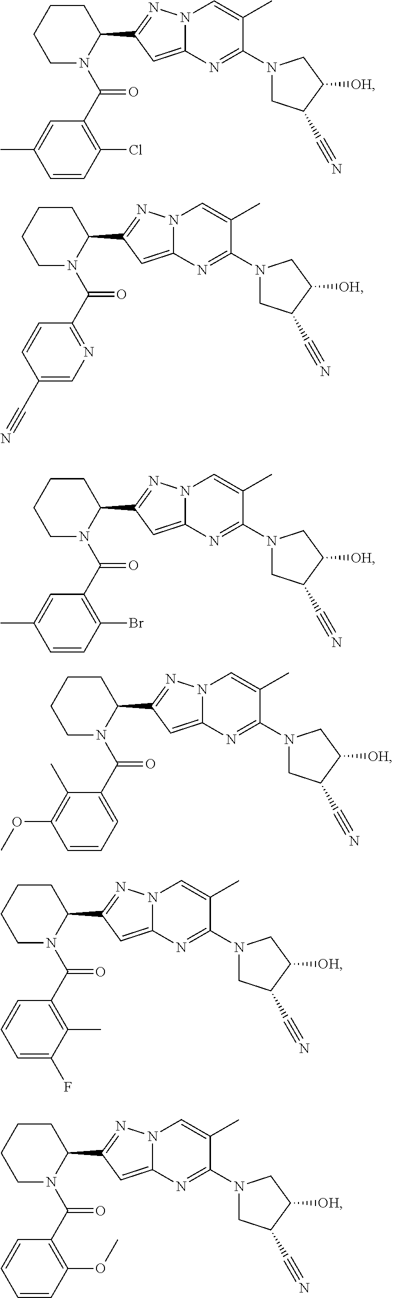

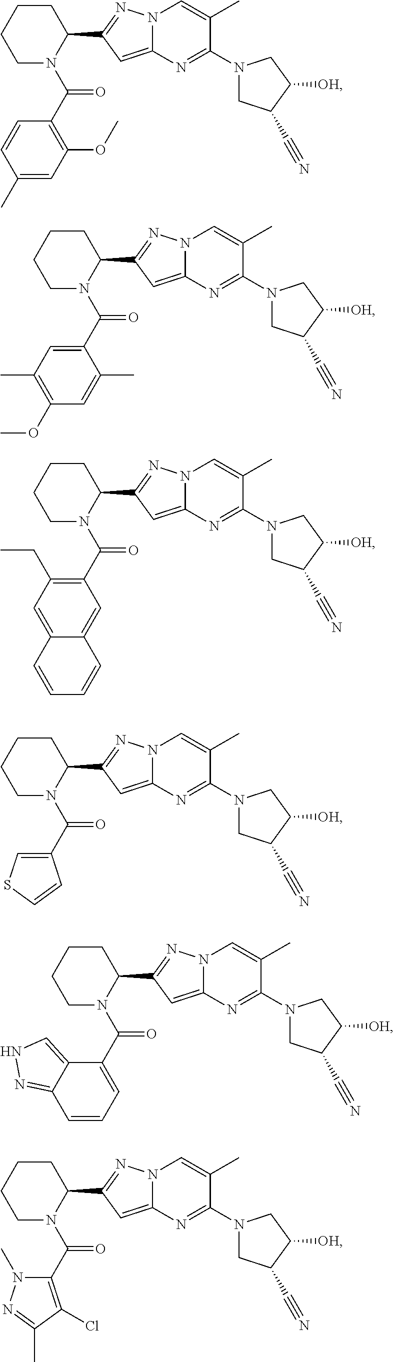

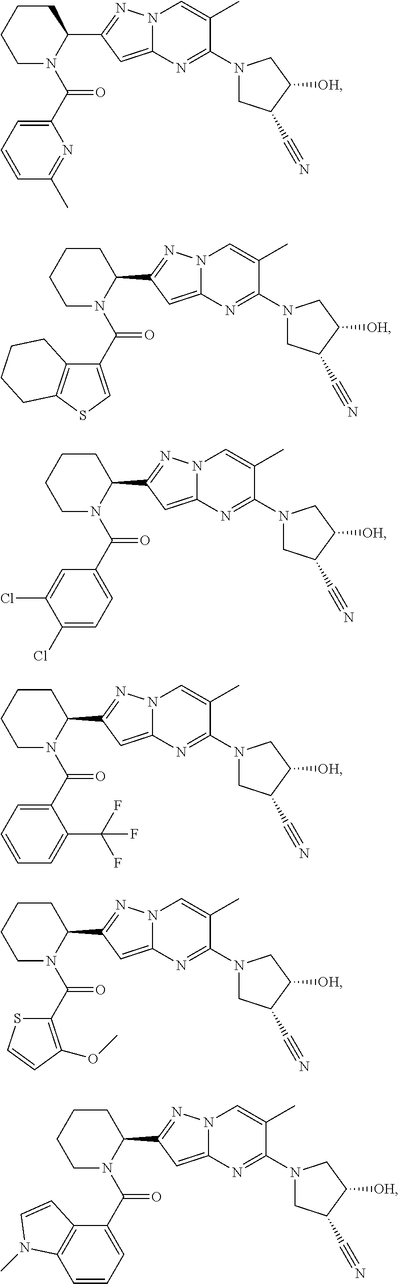

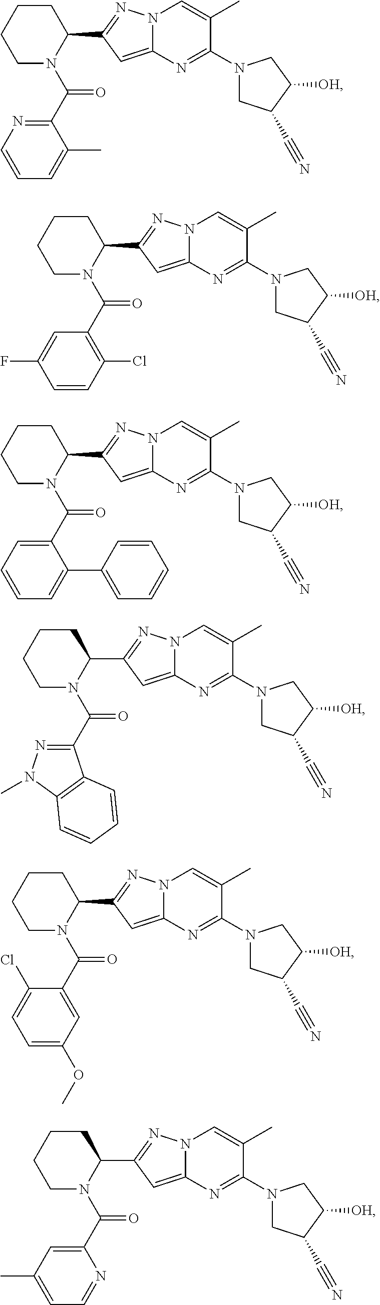

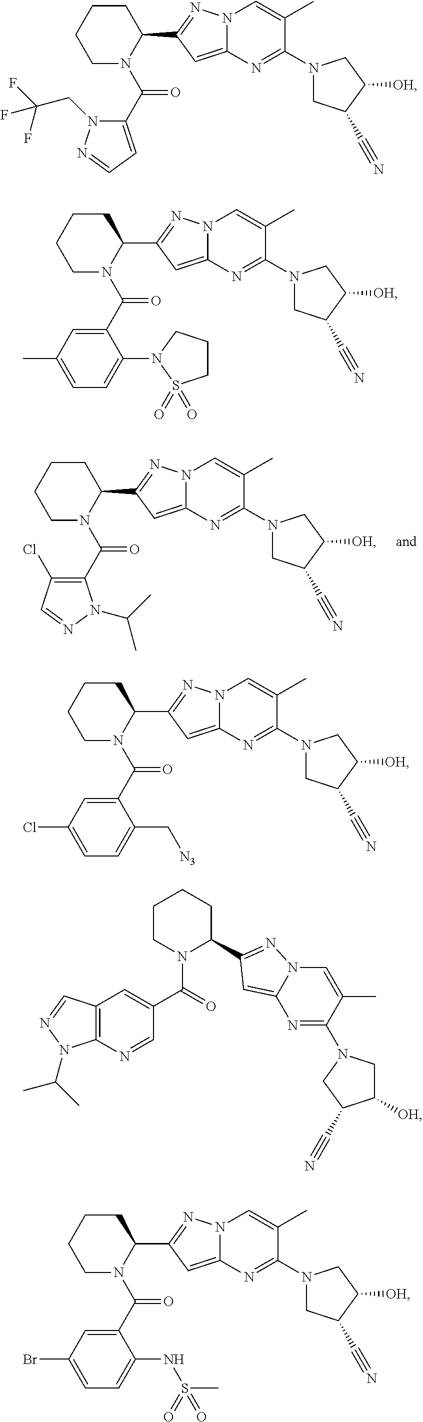

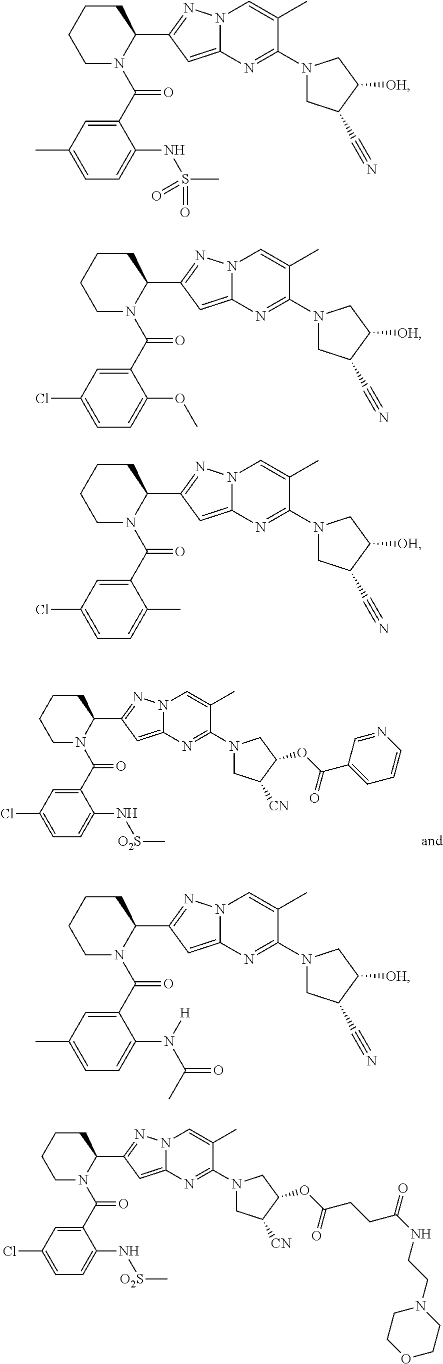

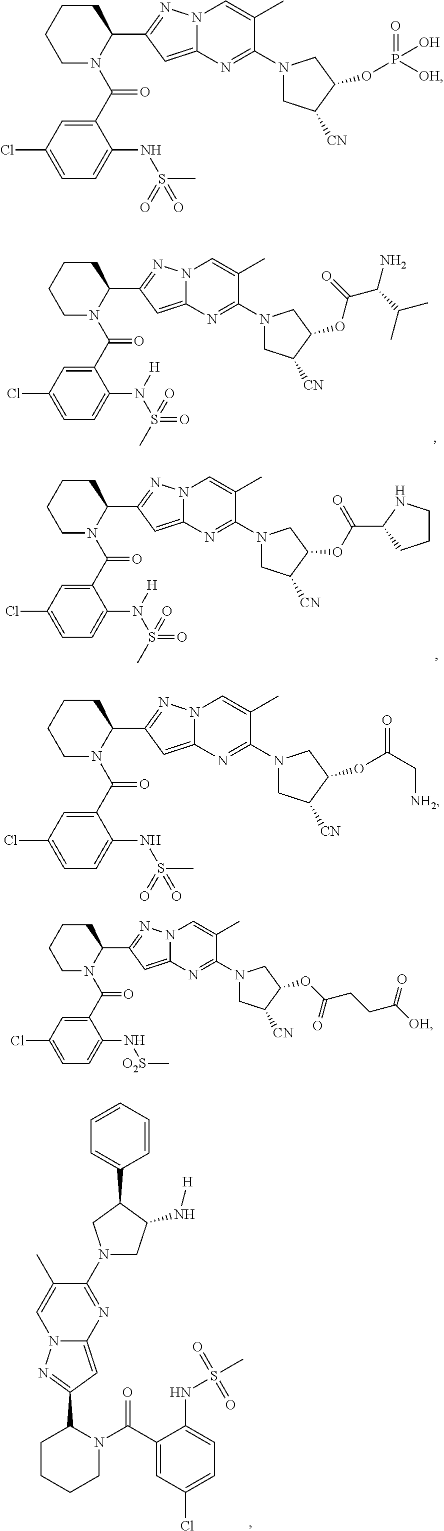

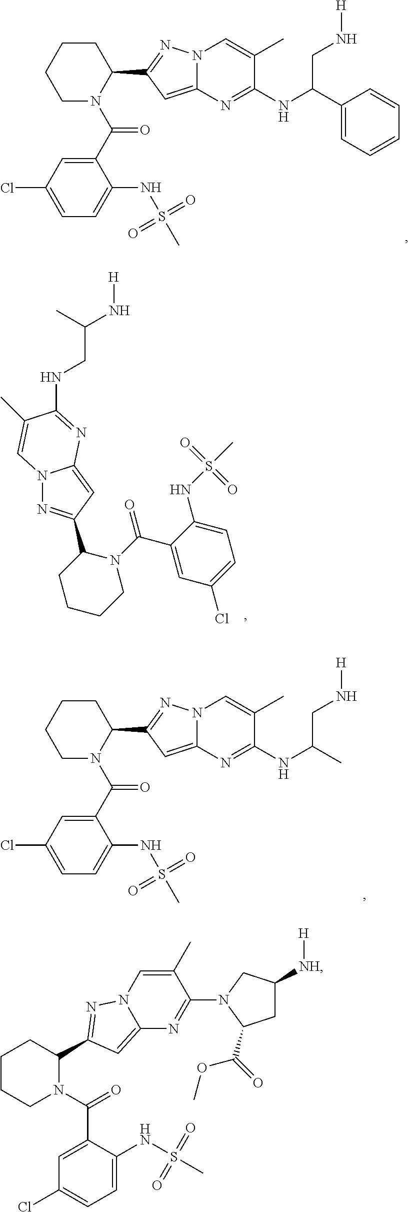

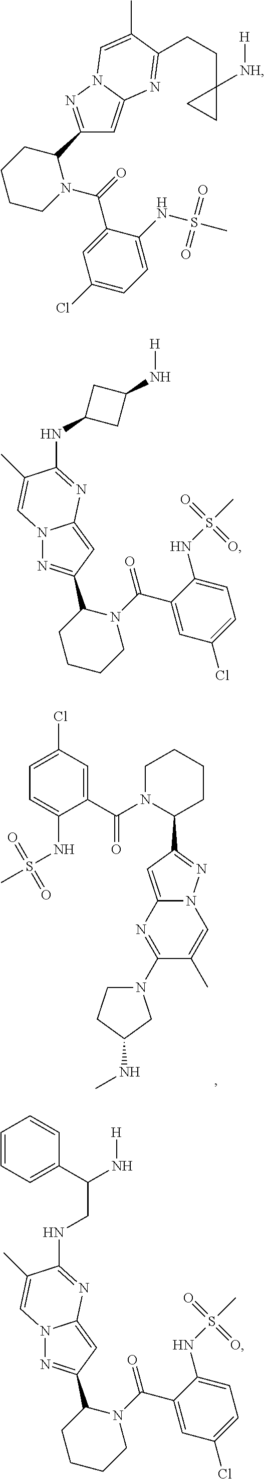

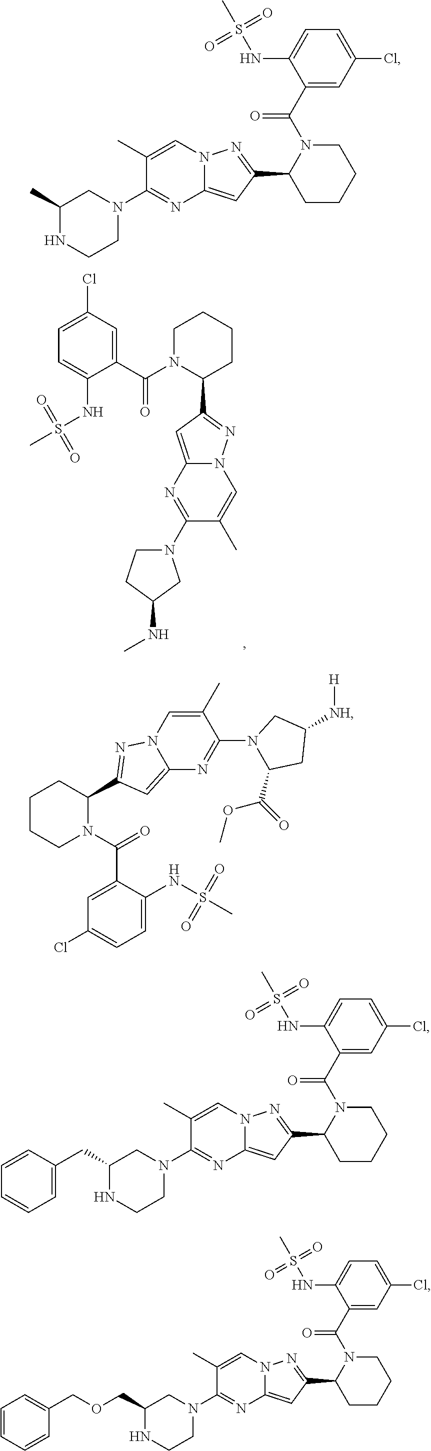

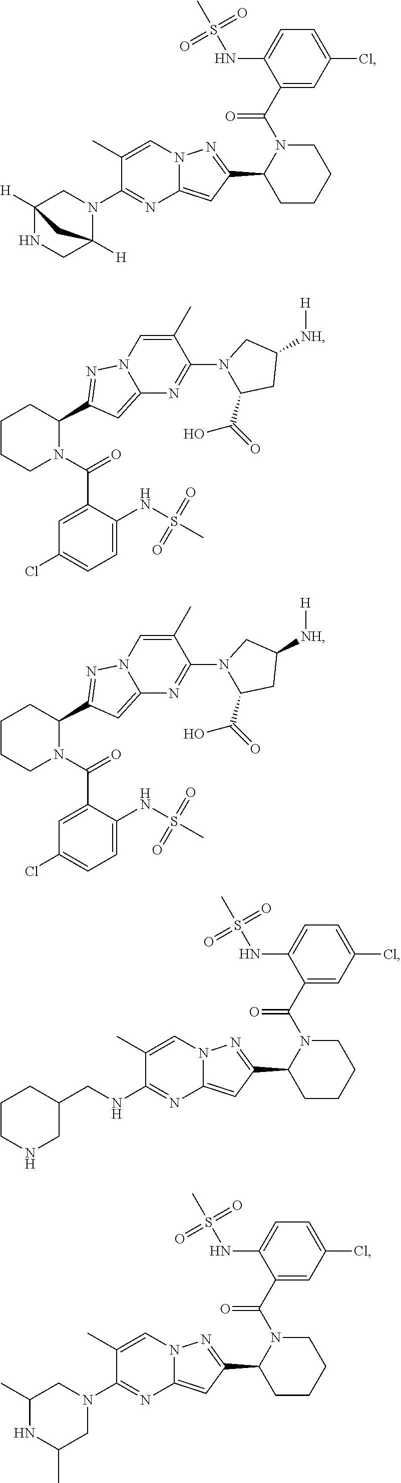 [SMILES: Cc1cn2nc([C@@H]3CCCCN3C(=O)c3cc(Cl)ccc3NS(C)(=O)=O)cc2nc1N1C[C@@H]2C[C@H]1CN2 ; Cc1cn2nc([C@@H]3CCCCN3C(=O)c3cc(Cl)ccc3NS(C)(=O)=O)cc2nc1N1C[C@H](N)C[C@@H]1C(=O)O ; Cc1cn2nc([C@@H]3CCCCN3C(=O)c3cc(Cl)ccc3NS(C)(=O)=O)cc2nc1N1C[C@@H](N)C[C@@H]1C(=O)O ; Cc1cn2nc([C@@H]3CCCCN3C(=O)c3cc(Cl)ccc3NS(C)(=O)=O)cc2nc1NCC1CCCNC1 ; Cc1cn2nc([C@@H]3CCCCN3C(=O)c3cc(Cl)ccc3NS(C)(=O)=O)cc2nc1N1CC(C)NC(C)C1]

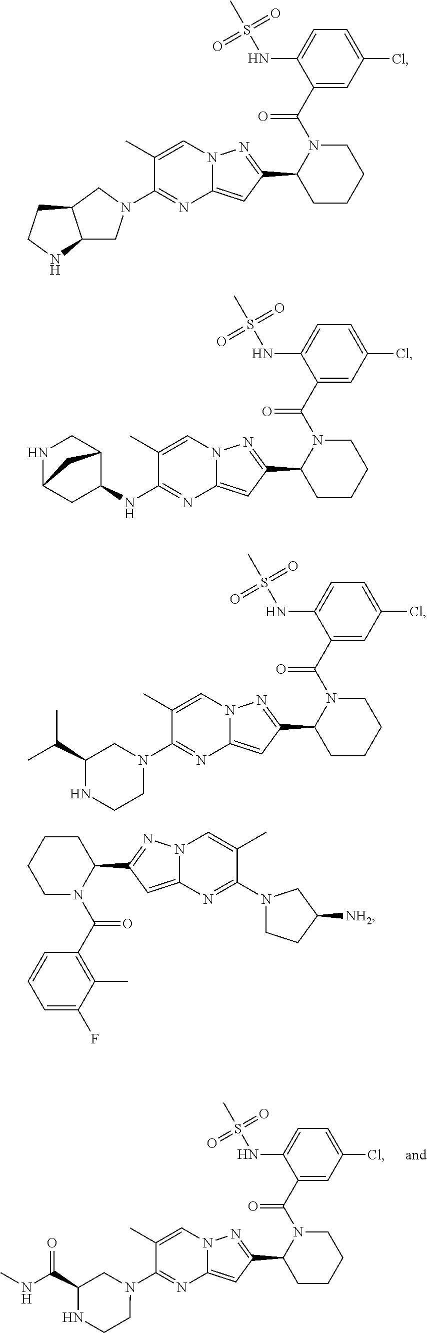

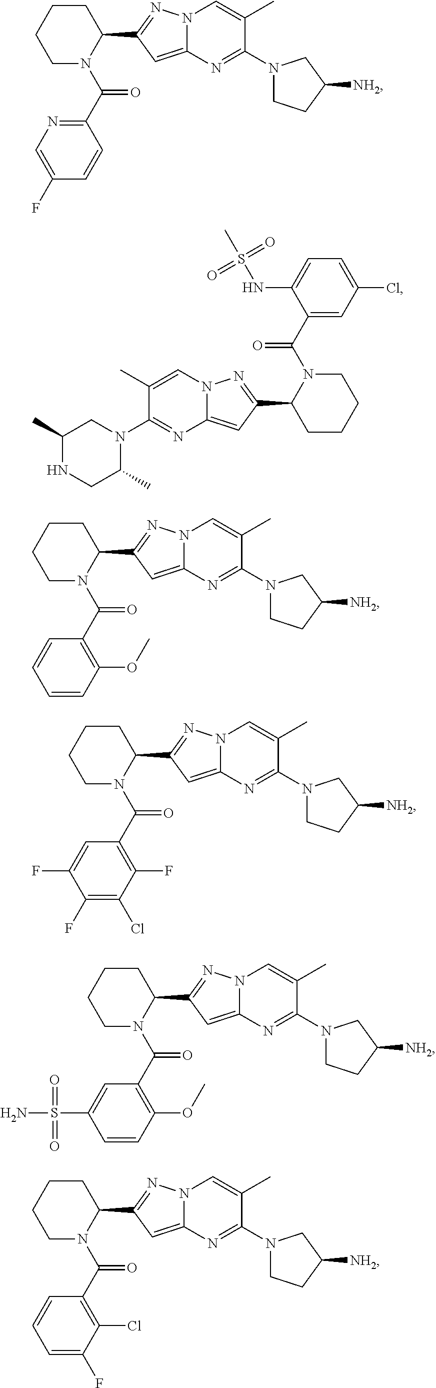

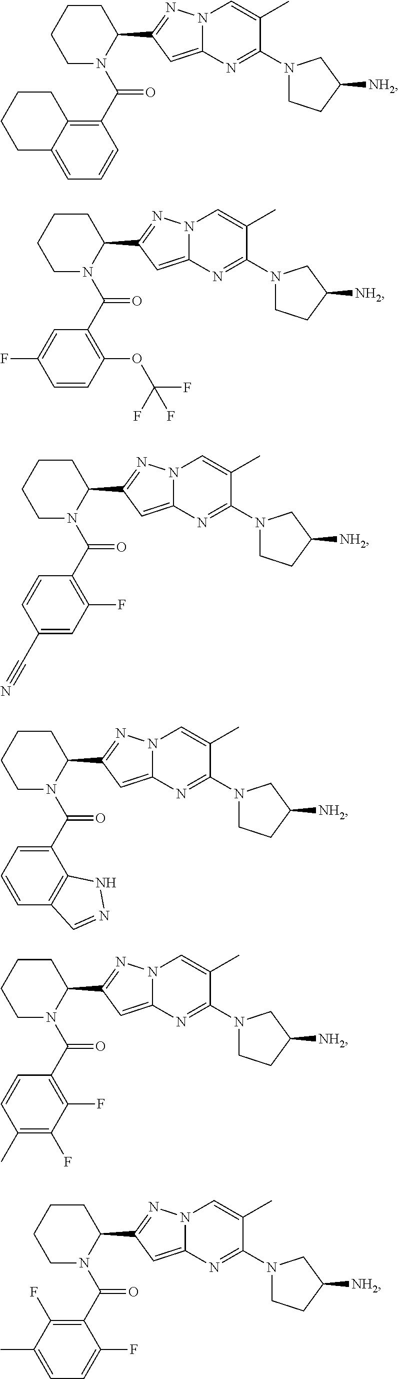

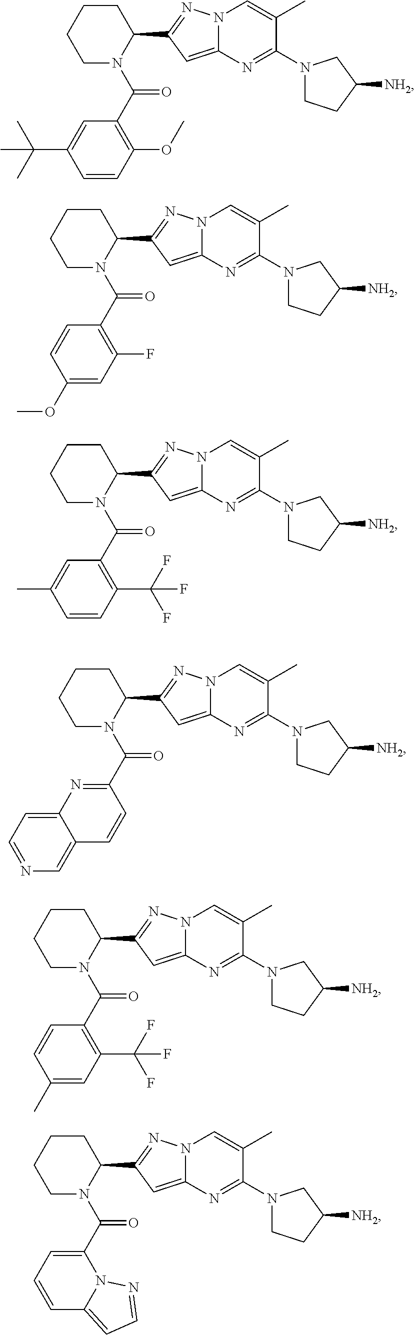

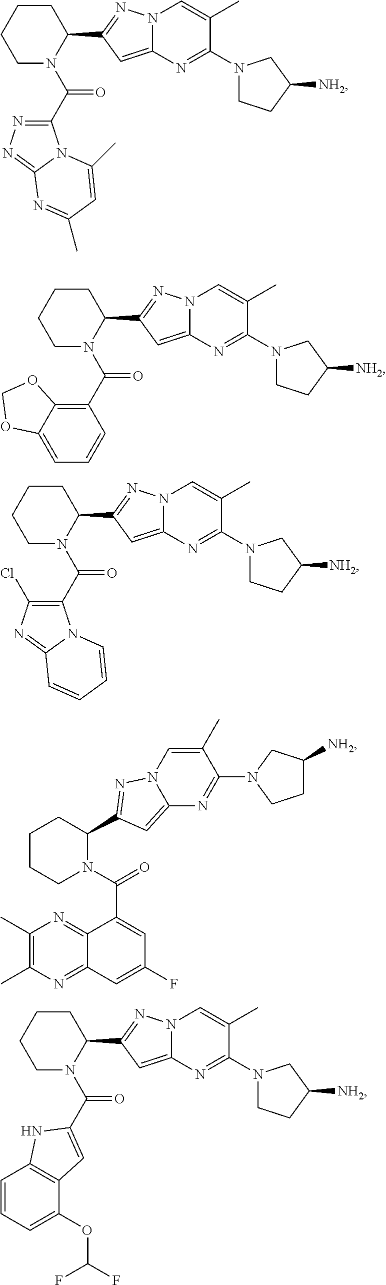

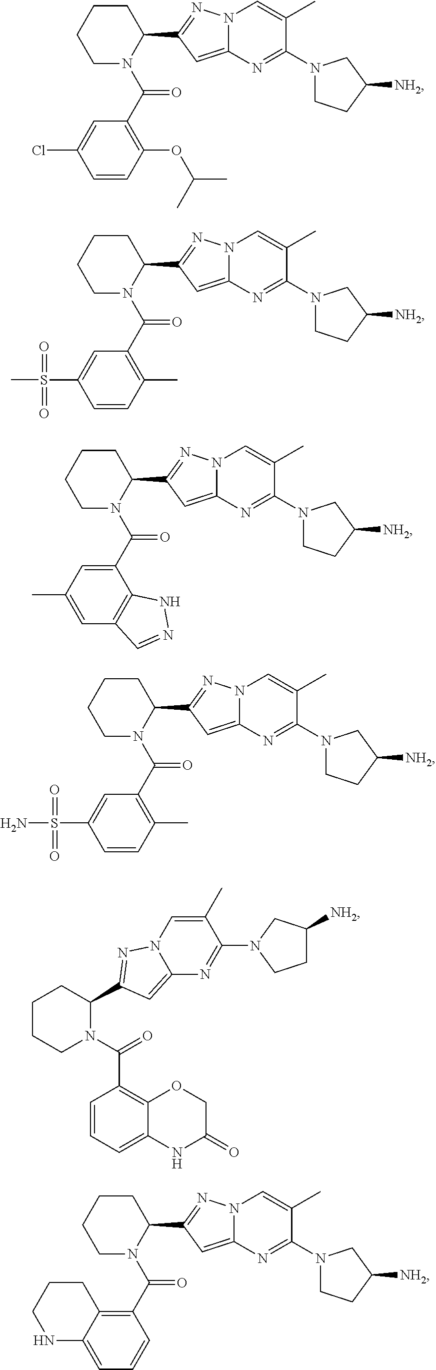

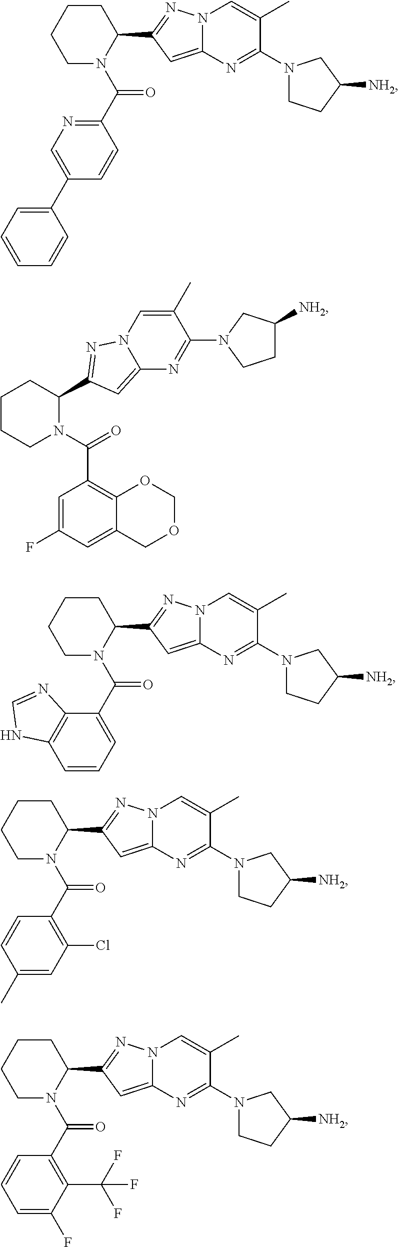

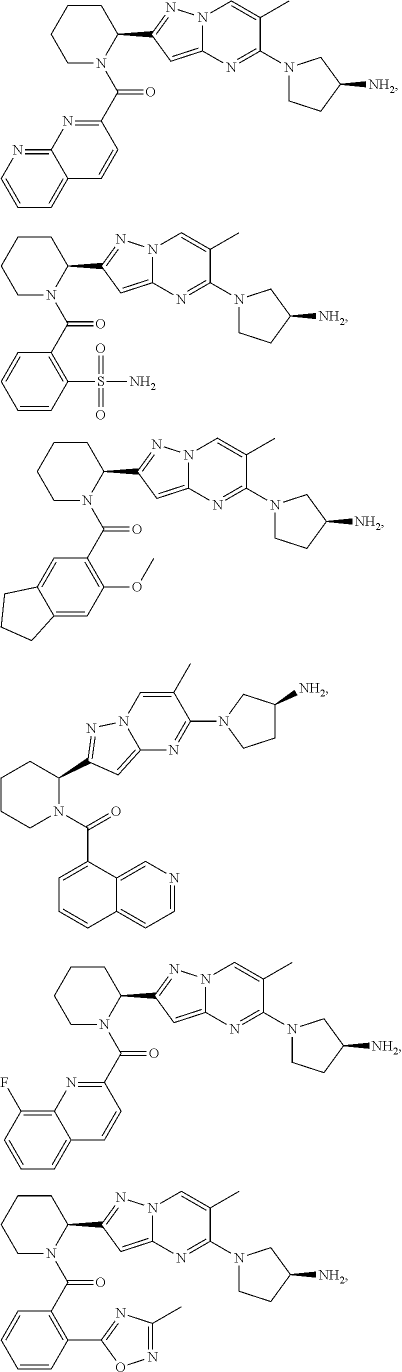

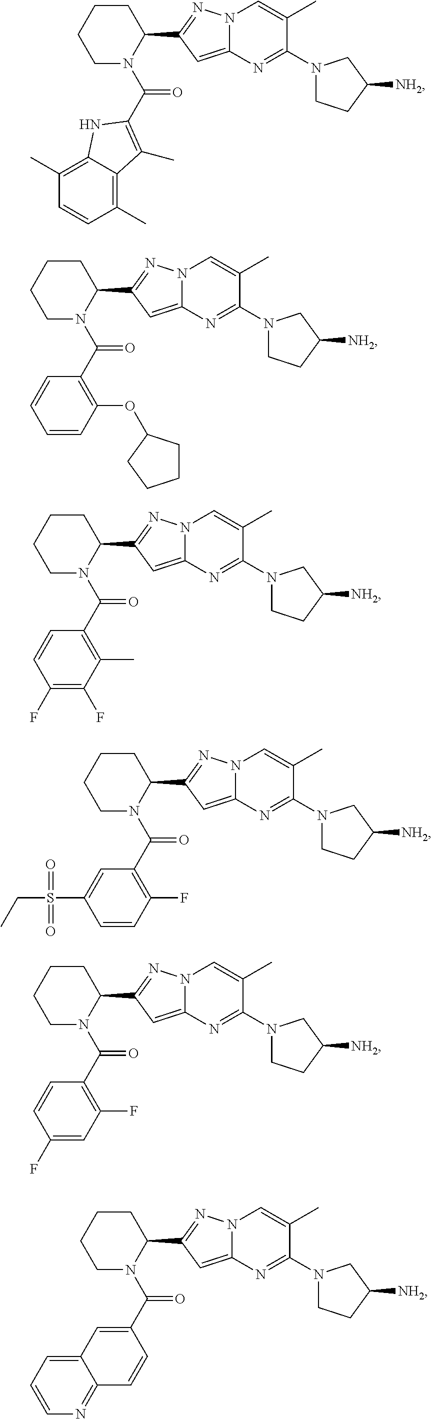

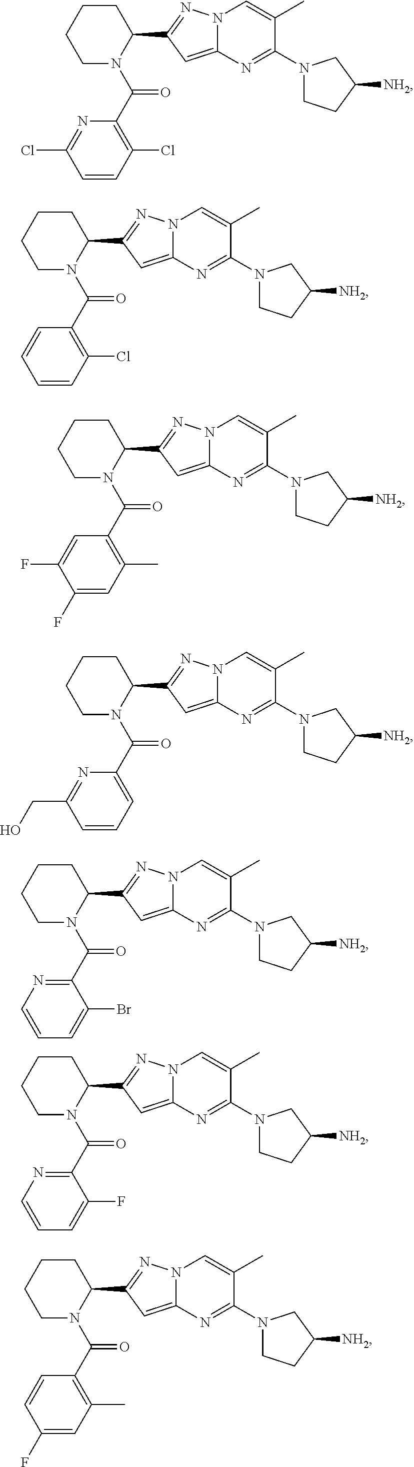

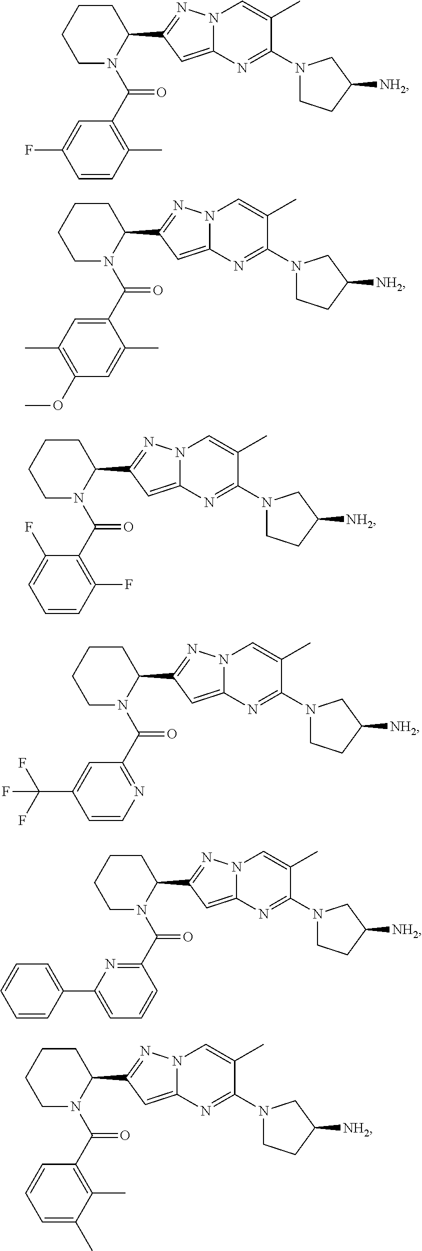

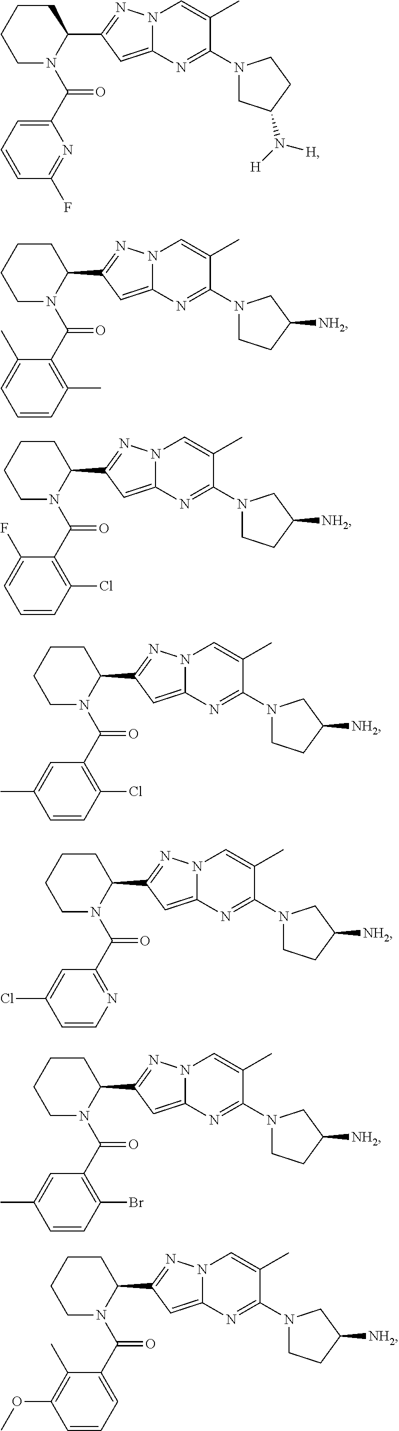

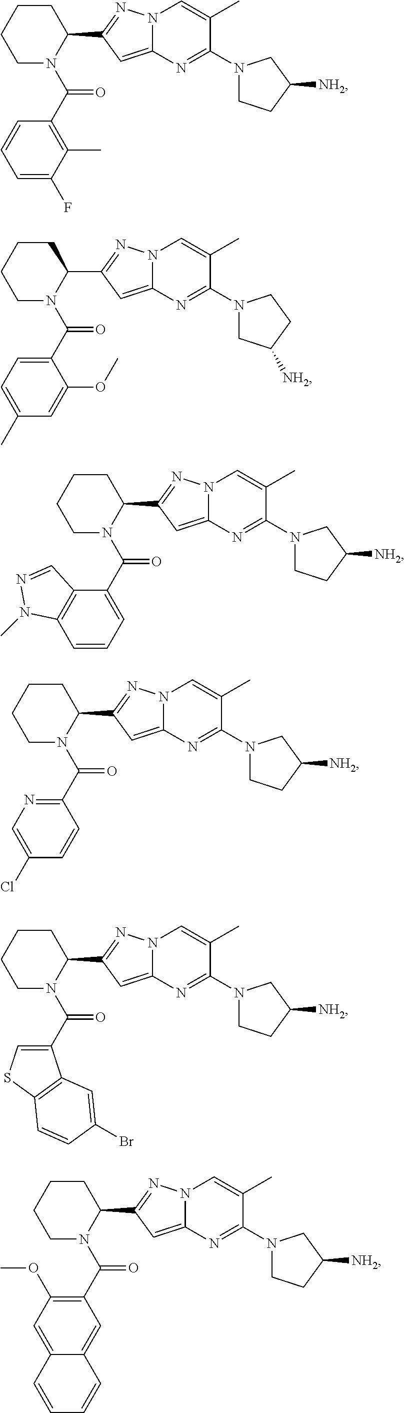

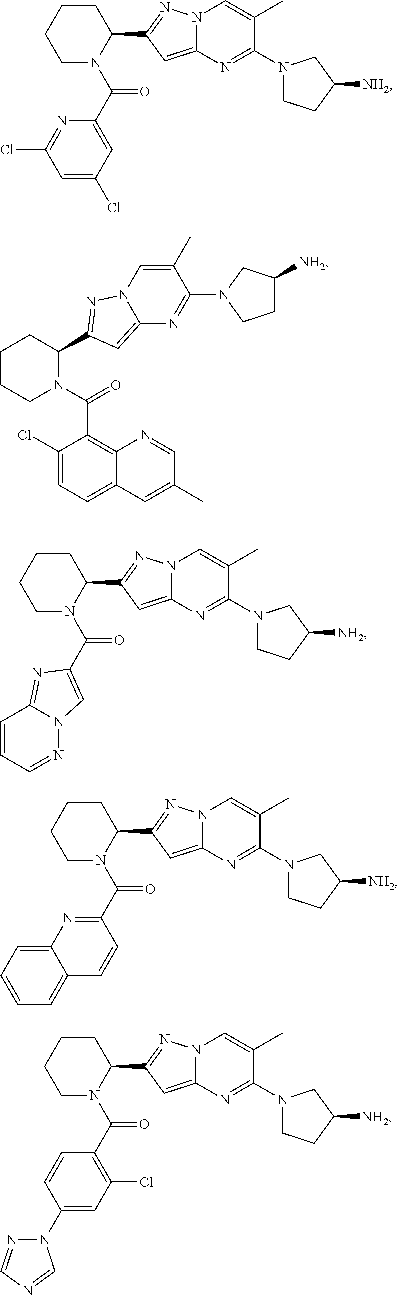

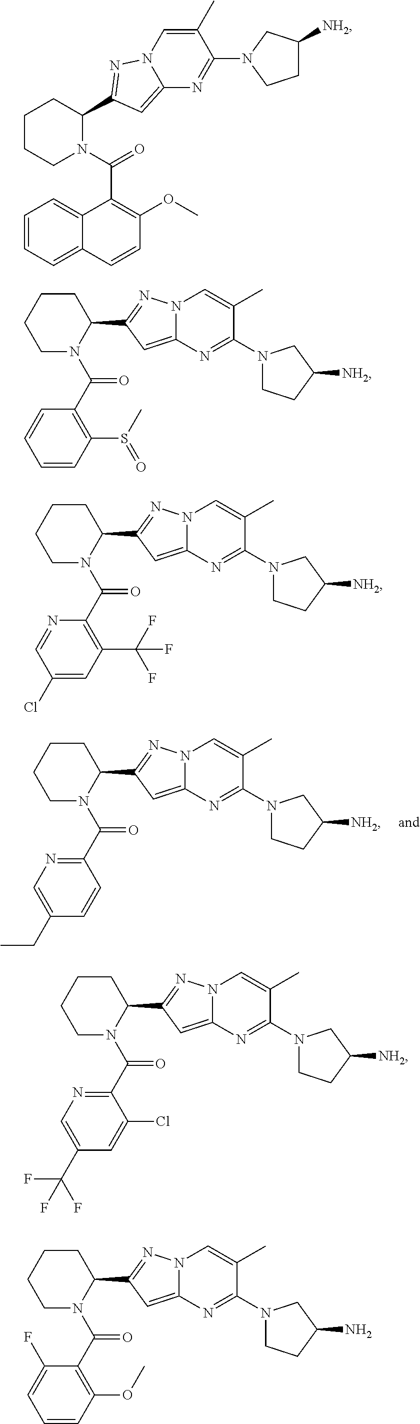

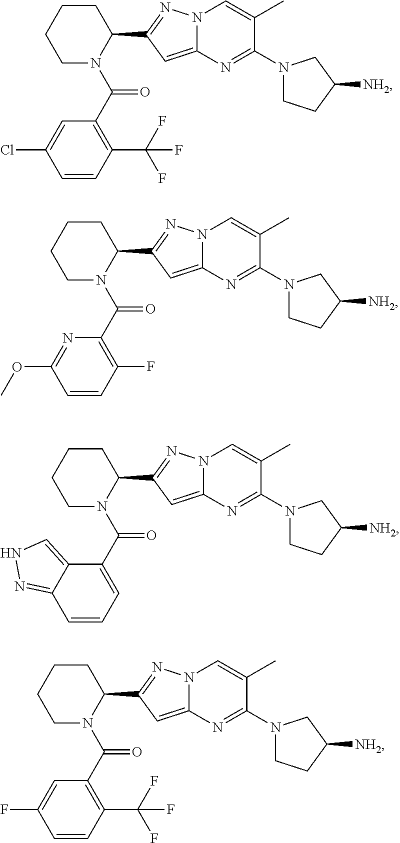

### Compounds of Formula (B20)

Compounds of the general Formula (B20) are described in U.S. Publication No. 2014/0072554, published Mar. 13, 2014, which is hereby incorporated by reference in its entirety. Formula (B20) has a structure selected from:

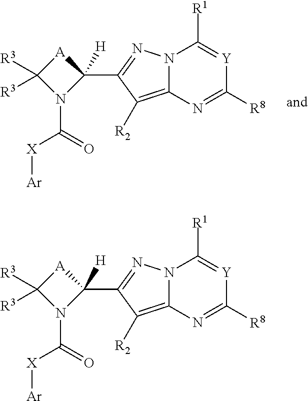

a pharmaceutically acceptable salt or ester, wherein: A can be —(C(R⁴)₂)ₙ— wherein any one C(R⁴)₂ of said —(C(R⁴)₂)ₙ— may be optionally replaced with —O—, —S—, —S(O)ₚ—, NH or NR^{a}; n can be 3, 4, 5 or 6; each p can be 1 or 2; Ar can be a C₂-C₂₀ heterocyclyl group or a C₆-C₂₀ aryl group, wherein the C₂-C₂₀ heterocyclyl group or the C₆-C₂₀ aryl group is optionally substituted with 1 to 5 R⁶; X can be —C(R¹³)(R¹⁴)—, —N(CH₂R¹⁴)— or X is absent; Y can be N or CR⁷; each R¹, R², R³, R⁴, R⁵, R⁶, R⁷ or R⁸ can be independently H, oxo, OR¹¹, NR¹¹R¹², NR¹¹C(O)R¹¹, NR¹¹C(O)OR¹¹, NR¹¹C(O)NR¹¹R¹², N₃, CN, NO₂, SR¹¹, S(O)ₚR^{a}, NR¹¹S(O)_{P}R^{a}, —C(═O)R¹¹, —C(═O)OR¹¹, —C(═O)NR¹¹R¹², C(═O)SR¹¹, —S(O)ₚ(OR¹¹), —SO₂NR¹¹R¹², NR¹¹S(O)ₚ(OR¹¹), —NR¹¹ SOₚNR¹¹R¹², NR¹¹C(═NR¹¹)NR¹¹R¹², halogen, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl; two R⁴ on adjacent carbon atoms, when taken together, may form a double bond between the two carbons to which they are attached or may form a (C₃-C₇)cycloalkyl ring wherein one carbon atom of said (C₃-C₇)cycloalkyl ring may be optionally replaced by —O—, —S—, —S(O)ₚ—, —NH— or —NR^{a}—; four R⁴ on adjacent carbon atoms, when taken together, may form an optionally substituted C₆ aryl ring; two R⁴ on the same carbon atom, when taken together, may form a (C₃-C₇)cycloalkyl ring wherein one carbon atom of said (C₃-C₇)cycloalkyl ring may be optionally replaced by —O—, —S—, —S(O)ₚ—, —NH— or —NR^{a}—; two R⁶ on adjacent carbon atoms, when taken together, may form a (C₃-C₇)cycloalkyl ring wherein one carbon atom of said (C₃-C₇)cycloalkyl ring may be optionally replaced by —O—, —S—, —S(O)ₚ—, —NH— or —NR^{a}—; any R⁶ adjacent to the obligate carbonyl group of said Ar, when taken together with R³, may form a bond or a —(C(R⁵)₂)ₘ— group wherein m is 1 or 2; any R⁶ adjacent to the obligate carbonyl group of said Ar, when taken together with R², may form a bond; each R^{a} can be independently (C₁-C₈)alkyl, (C₁-C₈)haloalkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl wherein any (C₁-C₈)alkyl, (C₁-C₈)haloalkyl, (C₂-C₈)alkenyl or (C₂-C₈)alkynyl of R^{a} is optionally substituted with one or more OH, NH₂, CO₂H, C₂-C₂₀ heterocyclyl, and wherein any aryl(C₃-C₈)alkyl, C₆-C₂₀ aryl, C₂-C₂₀ heterocyclyl, (C₃-C₇)cycloalkyl or (C₄-C₈)carbocyclylalkyl of R^{a} is optionally substituted with one or more OH, NH₂, CO₂H, C₂-C₂₀ heterocyclyl or (C₁-C₈)alkyl; each R¹¹ or R¹² can be independently H, (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl, C₄-C₈)carbocyclylalkyl, —C(═O)R^{a}, —S(O)ₚR^{a}, or aryl(C₁-C₈)alkyl; or R¹¹ and R¹² can be taken together with a nitrogen to which they are both attached form a 3 to 7 membered heterocyclic ring wherein any one carbon atom of said heterocyclic ring can optionally be replaced with —O—, —S—, —S(O)ₚ—, —NH—, —NR^{a}— or —C(O)—; R¹³ can be H or (C₁-C₈)alkyl; R¹⁴ can be H, (C₁-C₈)alkyl, NR¹¹R¹², NR¹¹C(O)R¹¹, NR¹¹C(O)OR¹¹, NR¹¹C(O)NR¹¹R¹², —NR¹¹S(O)ₚR^{a}, NR¹¹S(O)ₚ(OR¹¹) or NR¹¹SOₚNR¹¹R¹²; and wherein each (C₁-C₈)alkyl, (C₂-C₈)alkenyl, (C₂-C₈)alkynyl, aryl(C₁-C₈)alkyl, C₆-C₂₀ aryl, C₂-C₂₀ heterocyclyl, (C₃-C₇)cycloalkyl or (C₄-C₈)carbocyclylalkyl of each R¹, R², R³, R⁴, R⁵, R⁶, R⁷, R⁸, R¹¹ or R¹² can be, independently, optionally substituted with one or more oxo, halogen, hydroxy, NH₂, CN, N₃, N(R^{a})₂, NHR^{a}, SH, SR^{a}, S(O)ₚR^{a}, OR^{a}, (C₁-C₈)alkyl, (C₁-C₈)haloalkyl, —C(O)R^{a}, —C(O)H, —C(═O)OR^{a}, —C(═O)OH, —C(═O)N(R^{a})₂, —C(═O)NHR^{a}, —C(═O)NH₂, NHS(O)ₚR^{a}, NR^{a}S(O)ₚR^{a}, NHC(O)R^{a}, NR^{a}C(O)R^{a}, NHC(O)OR^{a}, NR^{a}C(O)OR^{a}, NR^{a}C(O)NHR^{a}, NR^{a}C(O)N(R^{a})₂, NR^{a}C(O)NH₂, NHC(O)NHR^{a}, NHC(O)N(R^{a})₂, NHC(O)NH₂, ═NH, ═NOH, ═NOR^{a}, NR^{a}S(O)ₚNHR^{a}, NR^{a}S(O)ₚN(R^{a})₂, NR^{a}S(O)ₚNH₂, NHS(O)ₚNHR^{a}, NHS(O)ₚN(R^{a})₂, NHS(O)ₚNH₂, —OC(═O)R^{a}, —OP(O)(OH)₂ or R^{a}.

Examples of Compounds of Formula (B20) include:

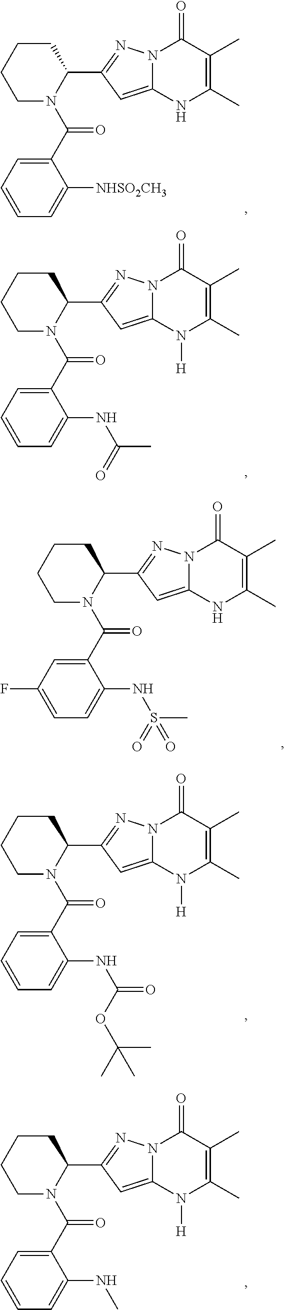

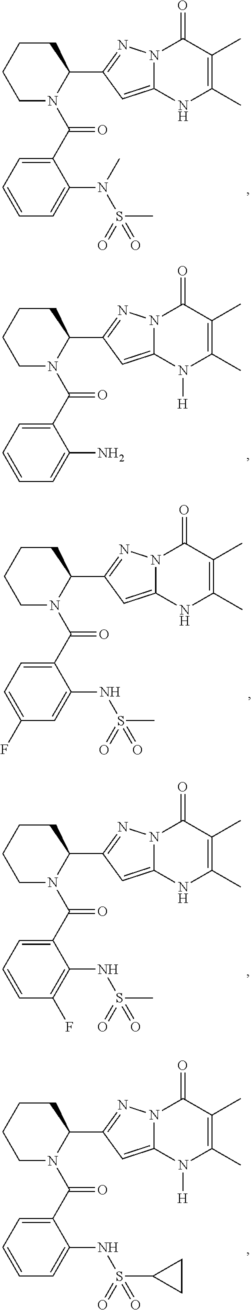 [SMILES: Cc1[nH]c2cc([C@@H]3CCCCN3C(=O)c3ccccc3N(C)S(C)(=O)=O)nn2c(=O)c1C ; Cc1[nH]c2cc([C@@H]3CCCCN3C(=O)c3ccccc3N)nn2c(=O)c1C ; Cc1[nH]c2cc([C@@H]3CCCCN3C(=O)c3ccc(F)cc3NS(C)(=O)=O)nn2c(=O)c1C ; Cc1[nH]c2cc([C@@H]3CCCCN3C(=O)c3cccc(F)c3NS(C)(=O)=O)nn2c(=O)c1C ; Cc1[nH]c2cc([C@@H]3CCCCN3C(=O)c3ccccc3NS(=O)(=O)C3CC3)nn2c(=O)c1C]

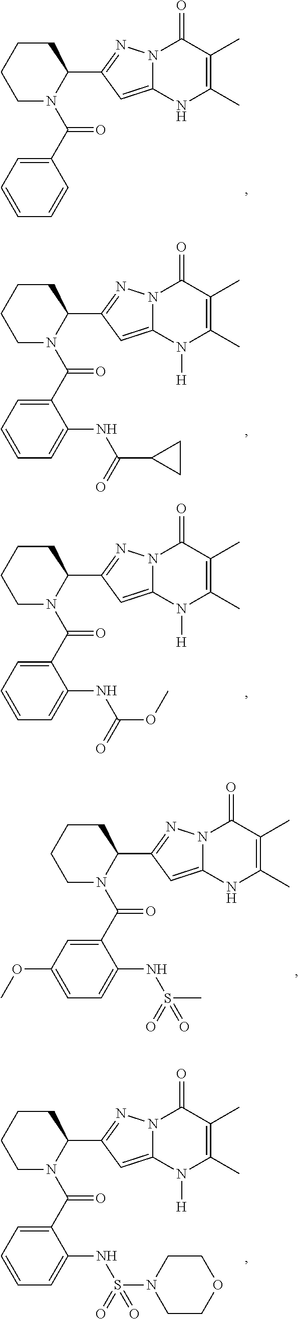

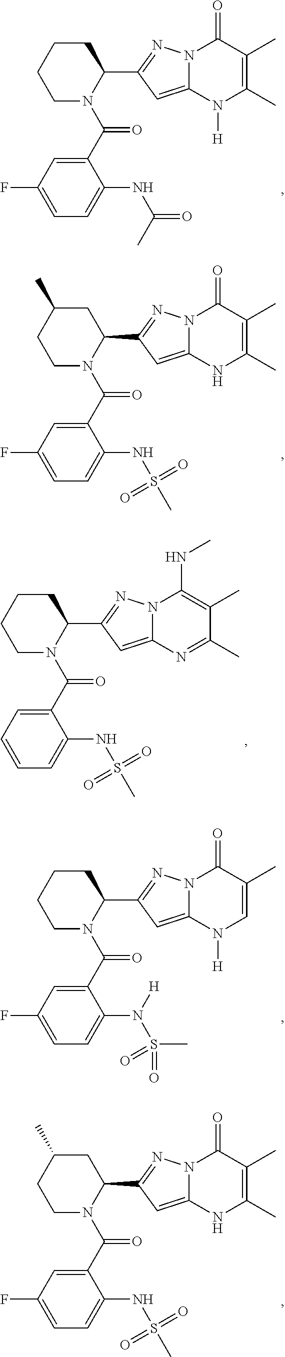 [SMILES: CC(=O)Nc1ccc(F)cc1C(=O)N1CCCC[C@H]1c1cc2[nH]c(C)c(C)c(=O)n2n1 ; Cc1[nH]c2cc([C@@H]3C[C@H](C)CCN3C(=O)c3cc(F)ccc3NS(C)(=O)=O)nn2c(=O)c1C ; CNc1c(C)c(C)nc2cc([C@@H]3CCCCN3C(=O)c3ccccc3NS(C)(=O)=O)nn12 ; Cc1c[nH]c2cc([C@@H]3CCCCN3C(=O)c3cc(F)ccc3NS(C)(=O)=O)nn2c1=O ; Cc1[nH]c2cc([C@@H]3C[C@@H](C)CCN3C(=O)c3cc(F)ccc3NS(C)(=O)=O)nn2c(=O)c1C]

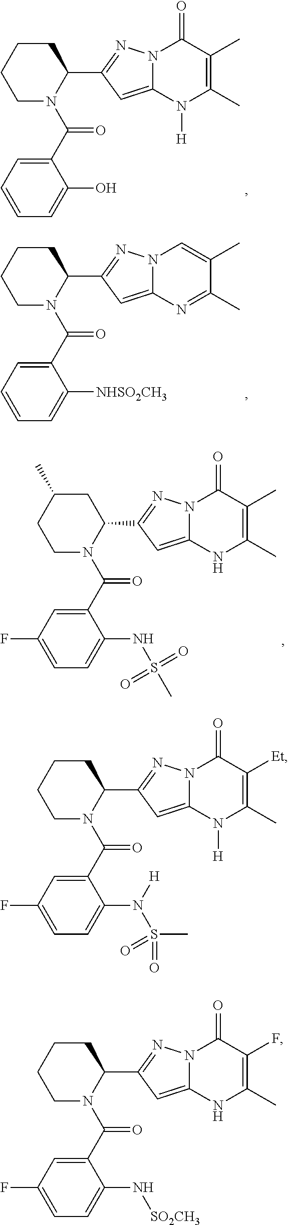

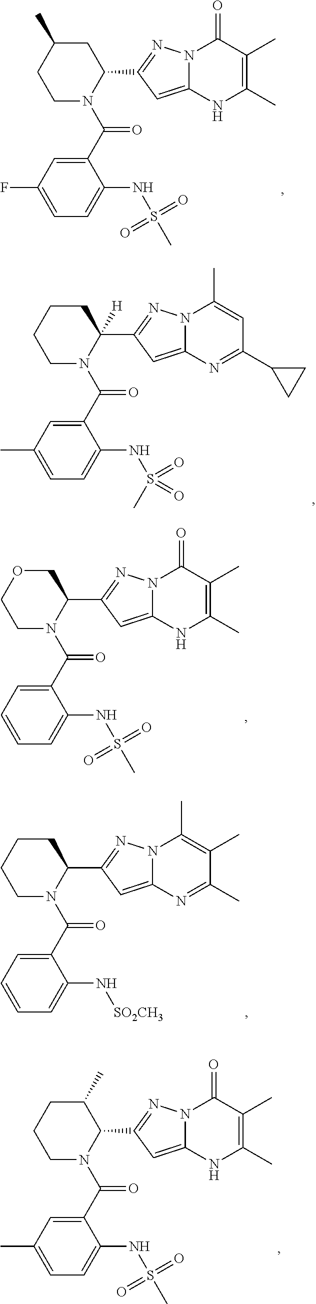

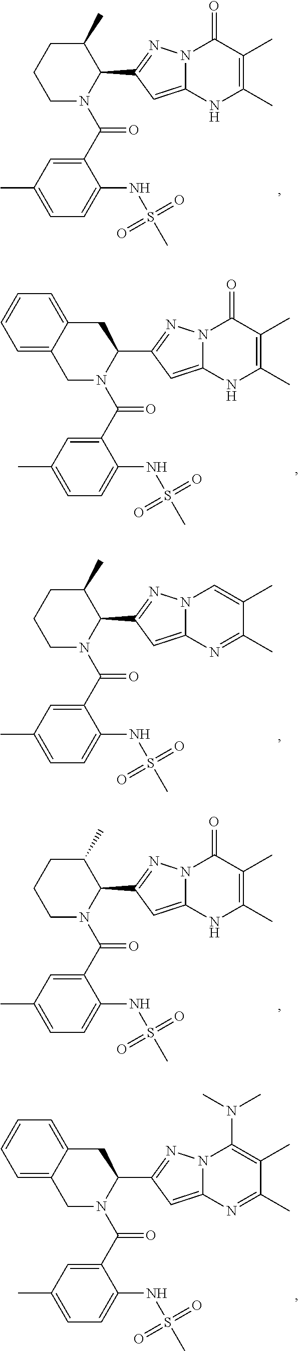

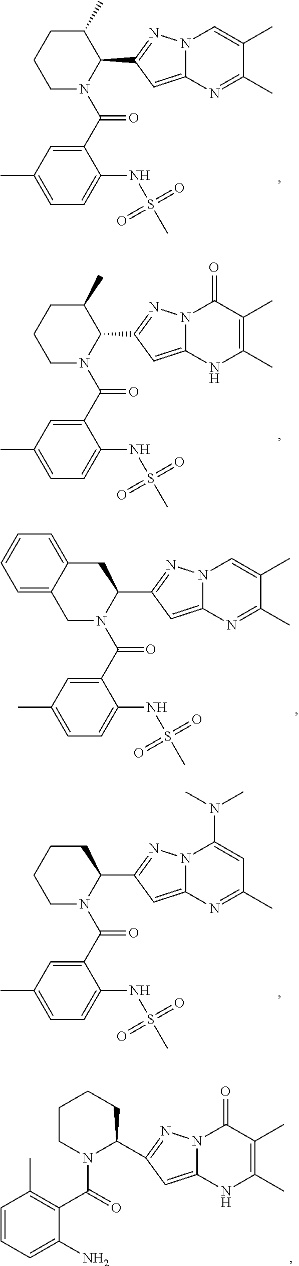

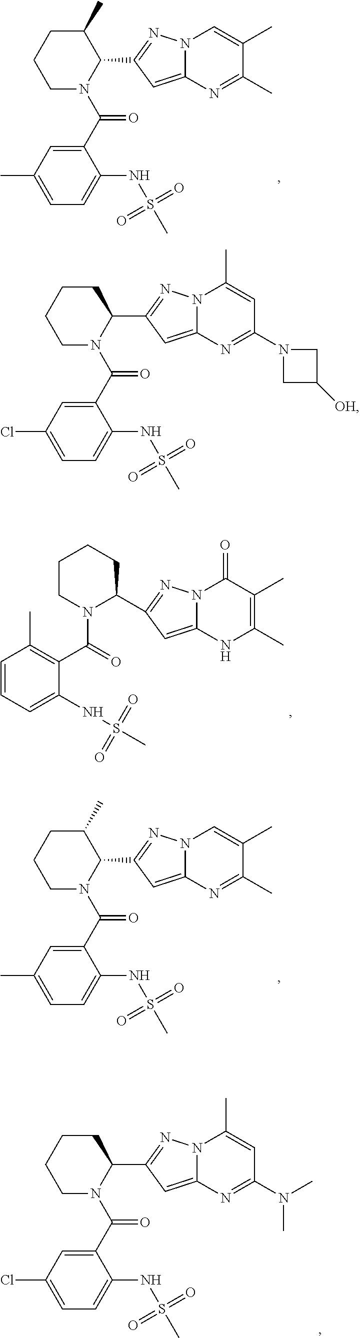

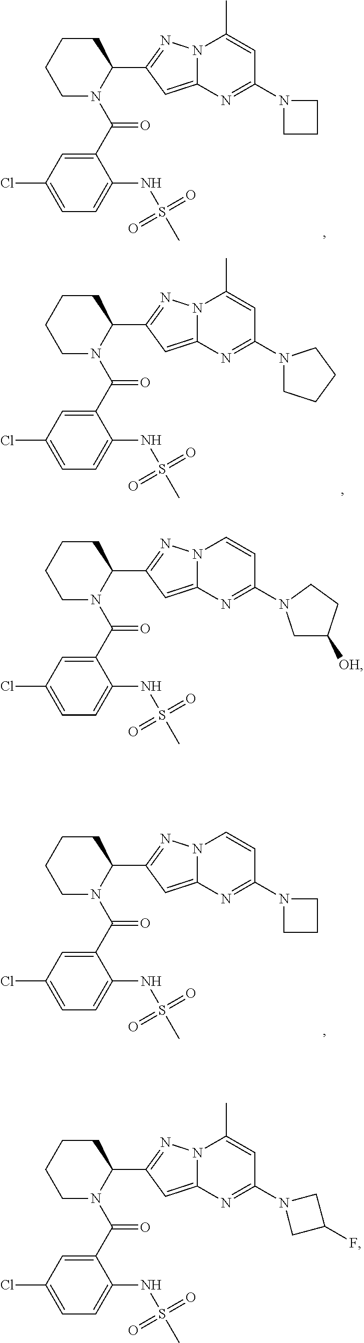 [SMILES: Cc1cc(N2CCC2)nc2cc([C@@H]3CCCCN3C(=O)c3cc(Cl)ccc3NS(C)(=O)=O)nn12 ; Cc1cc(N2CCCC2)nc2cc([C@@H]3CCCCN3C(=O)c3cc(Cl)ccc3NS(C)(=O)=O)nn12 ; CS(=O)(=O)Nc1ccc(Cl)cc1C(=O)N1CCCC[C@H]1c1cc2nc(N3CC[C@@H](O)C3)ccn2n1 ; CS(=O)(=O)Nc1ccc(Cl)cc1C(=O)N1CCCC[C@H]1c1cc2nc(N3CCC3)ccn2n1 ; Cc1cc(N2CC(F)C2)nc2cc([C@@H]3CCCCN3C(=O)c3cc(Cl)ccc3NS(C)(=O)=O)nn12]

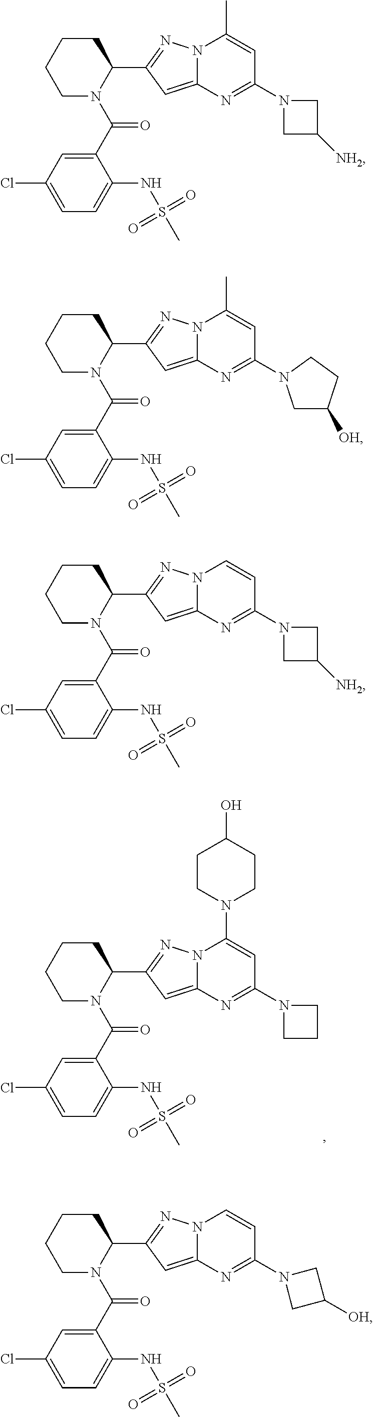 [SMILES: Cc1cc(N2CC(N)C2)nc2cc([C@@H]3CCCCN3C(=O)c3cc(Cl)ccc3NS(C)(=O)=O)nn12 ; Cc1cc(N2CC[C@@H](O)C2)nc2cc([C@@H]3CCCCN3C(=O)c3cc(Cl)ccc3NS(C)(=O)=O)nn12 ; CS(=O)(=O)Nc1ccc(Cl)cc1C(=O)N1CCCC[C@H]1c1cc2nc(N3CC(N)C3)ccn2n1 ; CS(=O)(=O)Nc1ccc(Cl)cc1C(=O)N1CCCC[C@H]1c1cc2nc(N3CCC3)cc(N3CCC(O)CC3)n2n1 ; CS(=O)(=O)Nc1ccc(Cl)cc1C(=O)N1CCCC[C@H]1c1cc2nc(N3CC(O)C3)ccn2n1]

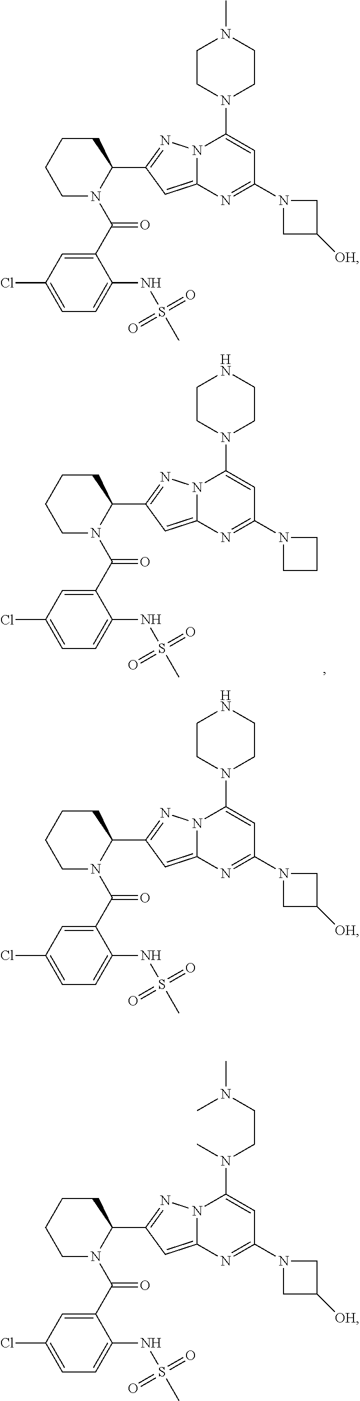 [SMILES: CN1CCN(c2cc(N3CC(O)C3)nc3cc([C@@H]4CCCCN4C(=O)c4cc(Cl)ccc4NS(C)(=O)=O)nn23)CC1 ; CS(=O)(=O)Nc1ccc(Cl)cc1C(=O)N1CCCC[C@H]1c1cc2nc(N3CCC3)cc(N3CCNCC3)n2n1 ; CS(=O)(=O)Nc1ccc(Cl)cc1C(=O)N1CCCC[C@H]1c1cc2nc(N3CC(O)C3)cc(N3CCNCC3)n2n1 ; CN(C)CCN(C)c1cc(N2CC(O)C2)nc2cc([C@@H]3CCCCN3C(=O)c3cc(Cl)ccc3NS(C)(=O)=O)nn12]

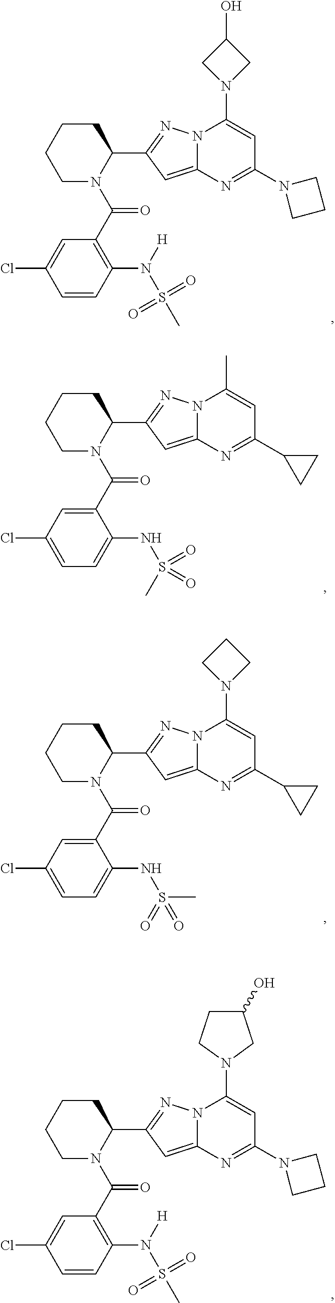 [SMILES: CS(=O)(=O)Nc1ccc(Cl)cc1C(=O)N1CCCC[C@H]1c1cc2nc(N3CCC3)cc(N3CC(O)C3)n2n1 ; Cc1cc(C2CC2)nc2cc([C@@H]3CCCCN3C(=O)c3cc(Cl)ccc3NS(C)(=O)=O)nn12 ; CS(=O)(=O)Nc1ccc(Cl)cc1C(=O)N1CCCC[C@H]1c1cc2nc(C3CC3)cc(N3CCC3)n2n1 ; CS(=O)(=O)Nc1ccc(Cl)cc1C(=O)N1CCCC[C@H]1c1cc2nc(N3CCC3)cc(N3CCC(O)C3)n2n1]

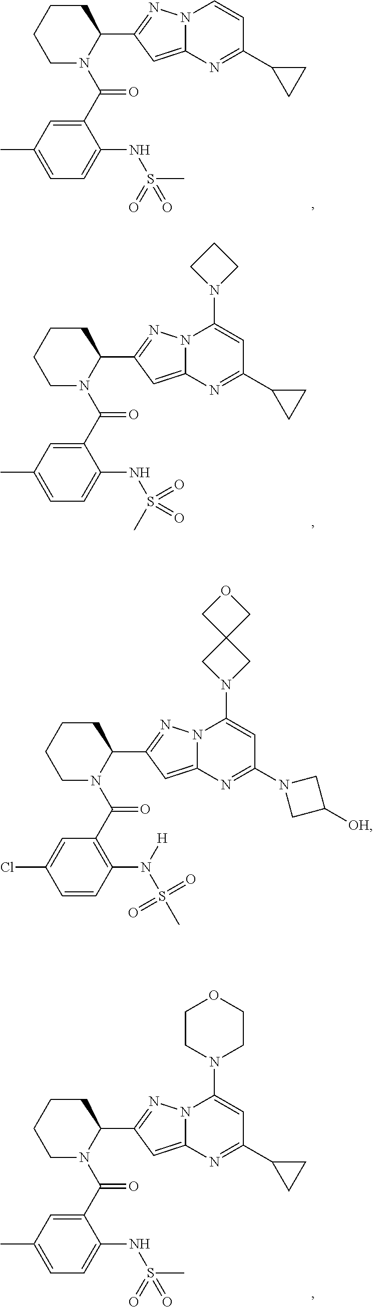 [SMILES: Cc1ccc(NS(C)(=O)=O)c(C(=O)N2CCCC[C@H]2c2cc3nc(C4CC4)ccn3n2)c1 ; Cc1ccc(NS(C)(=O)=O)c(C(=O)N2CCCC[C@H]2c2cc3nc(C4CC4)cc(N4CCC4)n3n2)c1 ; CS(=O)(=O)Nc1ccc(Cl)cc1C(=O)N1CCCC[C@H]1c1cc2nc(N3CC(O)C3)cc(N3CC4(COC4)C3)n2n1 ; Cc1ccc(NS(C)(=O)=O)c(C(=O)N2CCCC[C@H]2c2cc3nc(C4CC4)cc(N4CCOCC4)n3n2)c1]

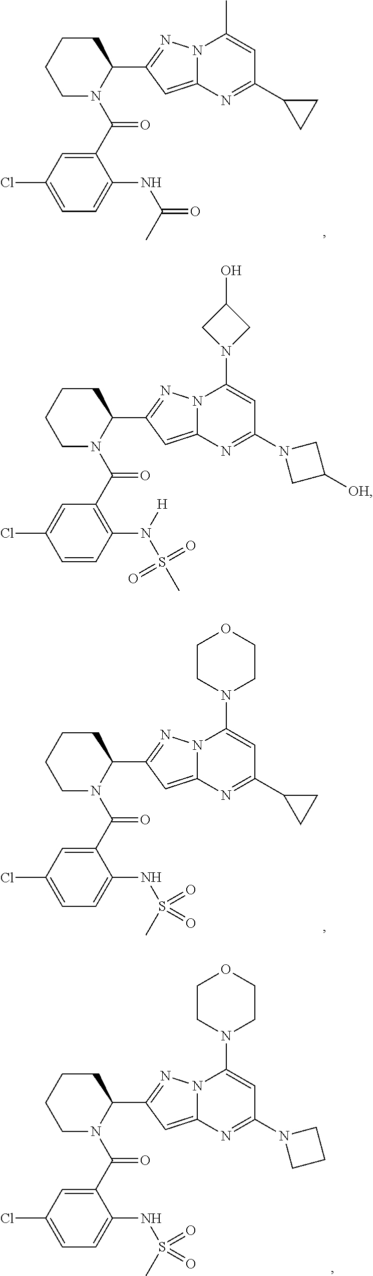 [SMILES: CC(=O)Nc1ccc(Cl)cc1C(=O)N1CCCC[C@H]1c1cc2nc(C3CC3)cc(C)n2n1 ; CS(=O)(=O)Nc1ccc(Cl)cc1C(=O)N1CCCC[C@H]1c1cc2nc(N3CC(O)C3)cc(N3CC(O)C3)n2n1 ; CS(=O)(=O)Nc1ccc(Cl)cc1C(=O)N1CCCC[C@H]1c1cc2nc(C3CC3)cc(N3CCOCC3)n2n1 ; CS(=O)(=O)Nc1ccc(Cl)cc1C(=O)N1CCCC[C@H]1c1cc2nc(N3CCC3)cc(N3CCOCC3)n2n1]

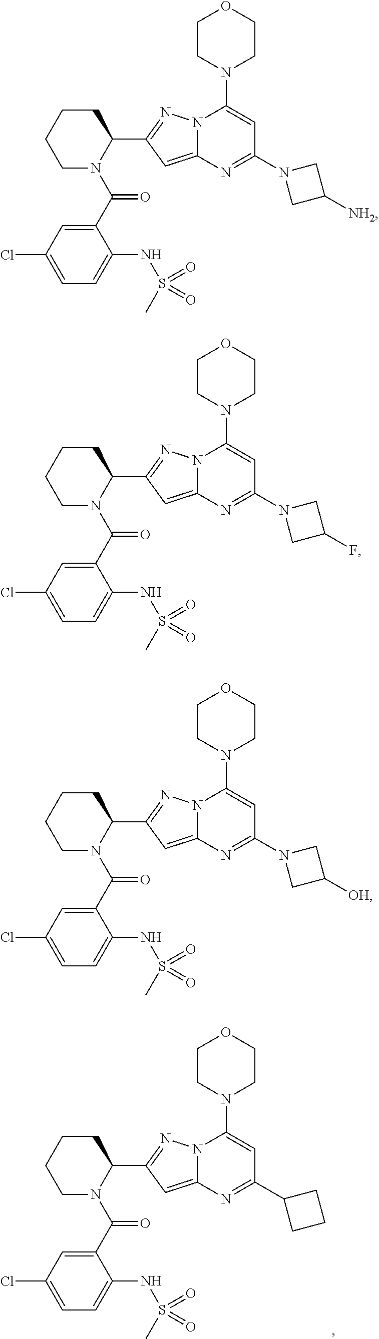 [SMILES: CS(=O)(=O)Nc1ccc(Cl)cc1C(=O)N1CCCC[C@H]1c1cc2nc(N3CC(N)C3)cc(N3CCOCC3)n2n1 ; CS(=O)(=O)Nc1ccc(Cl)cc1C(=O)N1CCCC[C@H]1c1cc2nc(N3CC(F)C3)cc(N3CCOCC3)n2n1 ; CS(=O)(=O)Nc1ccc(Cl)cc1C(=O)N1CCCC[C@H]1c1cc2nc(N3CC(O)C3)cc(N3CCOCC3)n2n1 ; CS(=O)(=O)Nc1ccc(Cl)cc1C(=O)N1CCCC[C@H]1c1cc2nc(C3CCC3)cc(N3CCOCC3)n2n1]

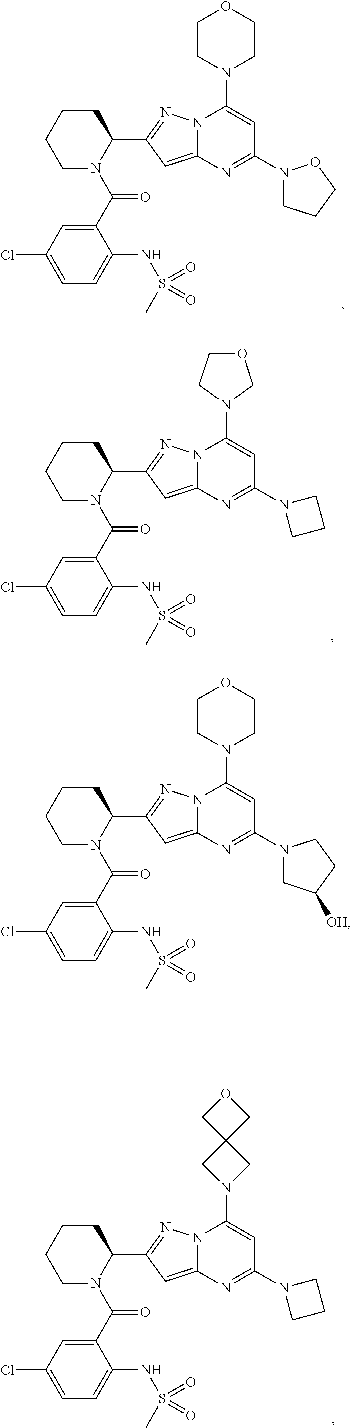 [SMILES: CS(=O)(=O)Nc1ccc(Cl)cc1C(=O)N1CCCC[C@H]1c1cc2nc(N3CCCO3)cc(N3CCOCC3)n2n1 ; CS(=O)(=O)Nc1ccc(Cl)cc1C(=O)N1CCCC[C@H]1c1cc2nc(N3CCC3)cc(N3CCOC3)n2n1 ; CS(=O)(=O)Nc1ccc(Cl)cc1C(=O)N1CCCC[C@H]1c1cc2nc(N3CC[C@@H](O)C3)cc(N3CCOCC3)n2n1 ; CS(=O)(=O)Nc1ccc(Cl)cc1C(=O)N1CCCC[C@H]1c1cc2nc(N3CCC3)cc(N3CC4(COC4)C3)n2n1]

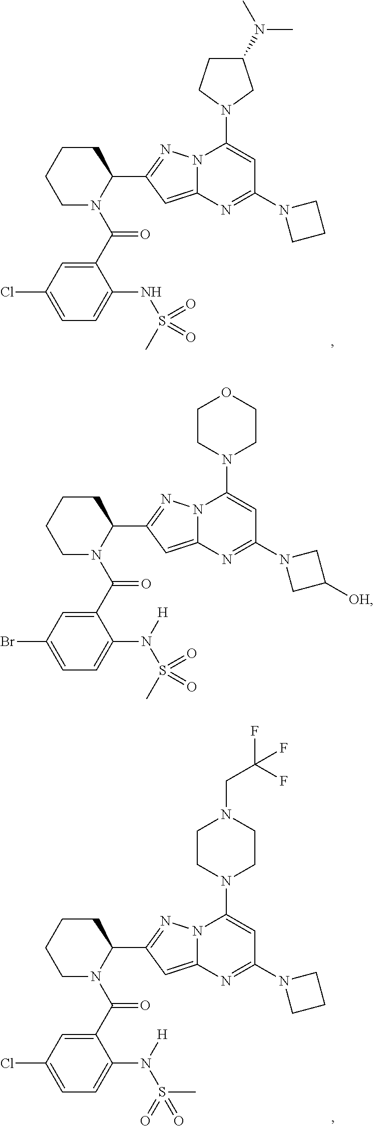 [SMILES: CN(C)[C@H]1CCN(c2cc(N3CCC3)nc3cc([C@@H]4CCCCN4C(=O)c4cc(Cl)ccc4NS(C)(=O)=O)nn23)C1 ; CS(=O)(=O)Nc1ccc(Br)cc1C(=O)N1CCCC[C@H]1c1cc2nc(N3CC(O)C3)cc(N3CCOCC3)n2n1 ; CS(=O)(=O)Nc1ccc(Cl)cc1C(=O)N1CCCC[C@H]1c1cc2nc(N3CCC3)cc(N3CCN(CC(F)(F)F)CC3)n2n1]

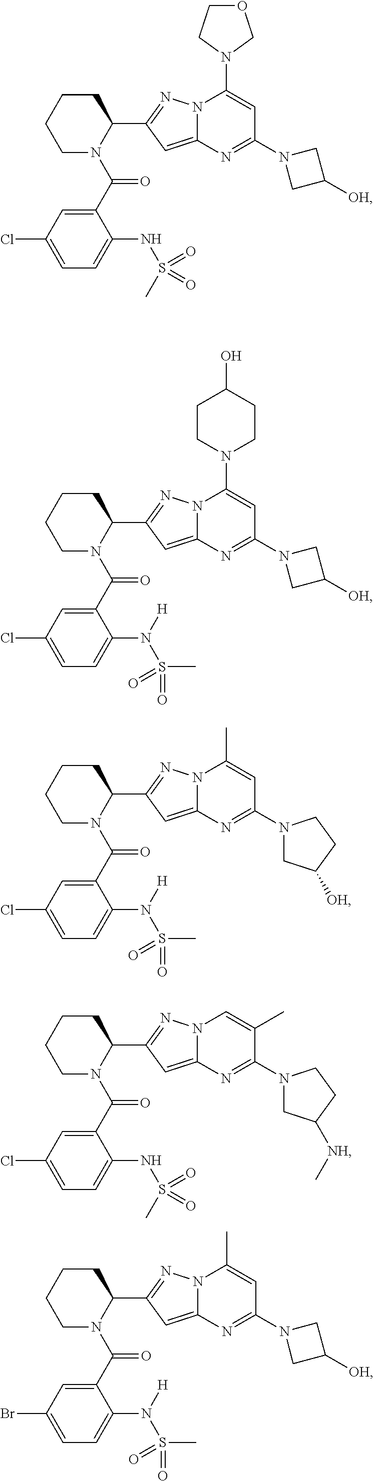

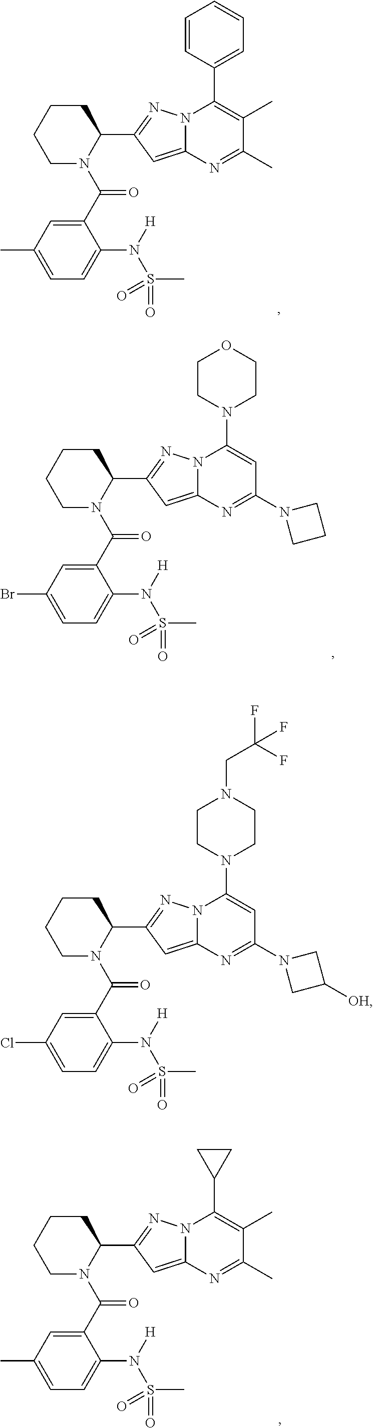

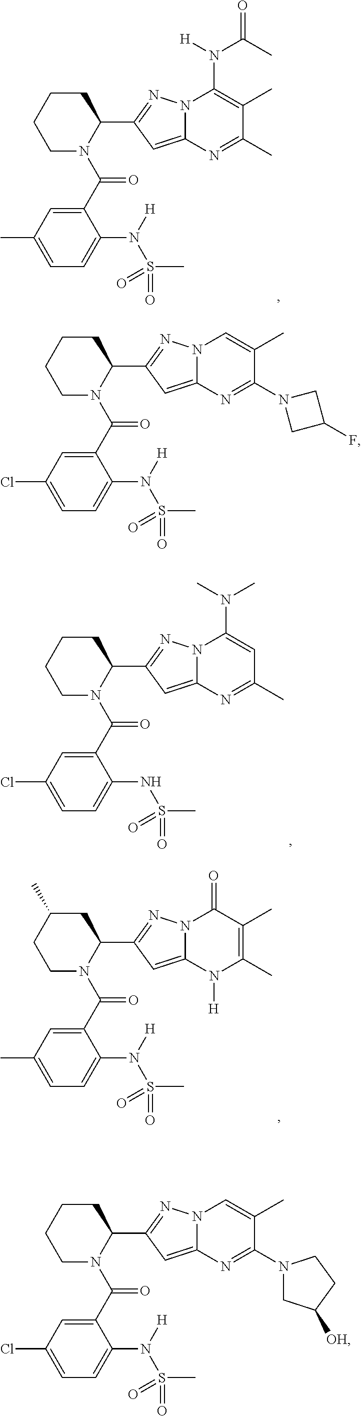

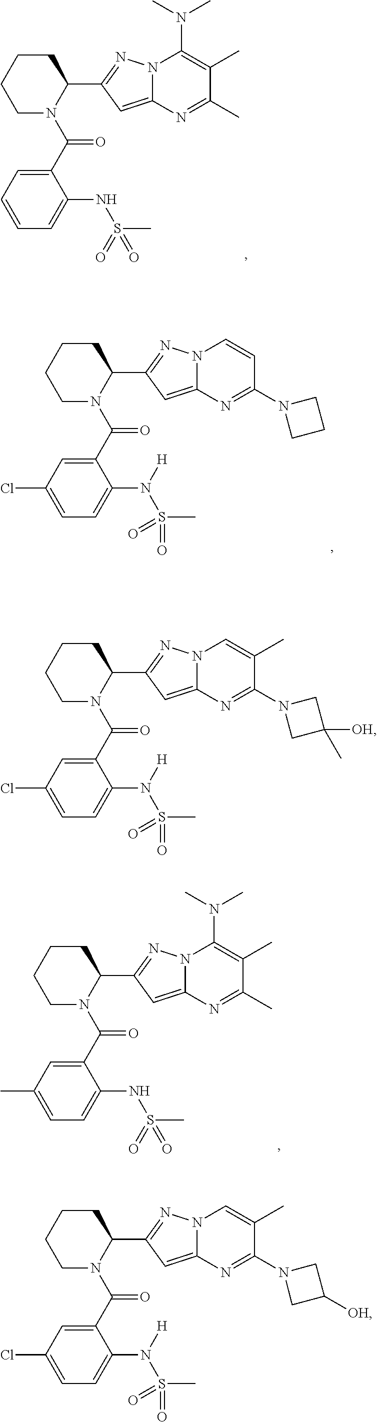

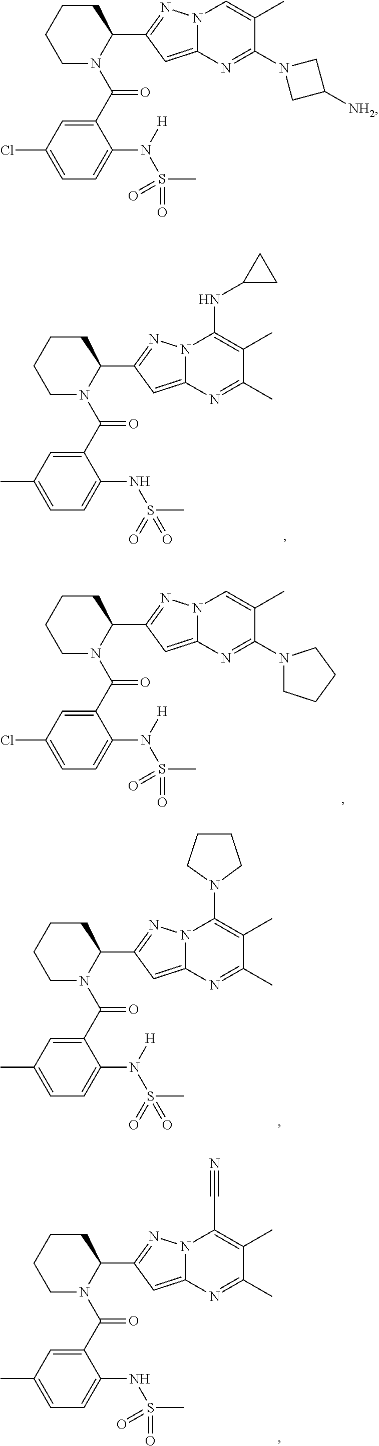

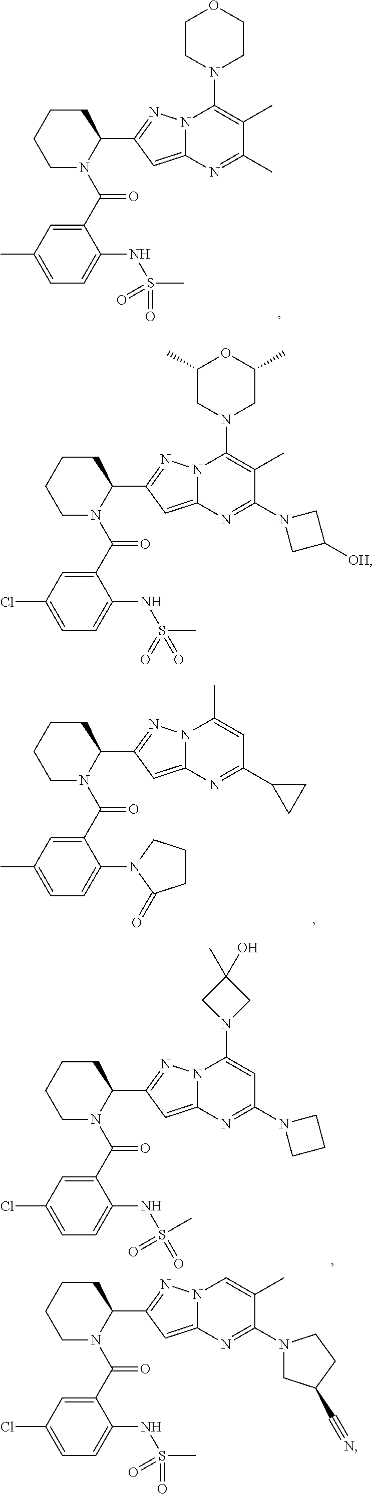

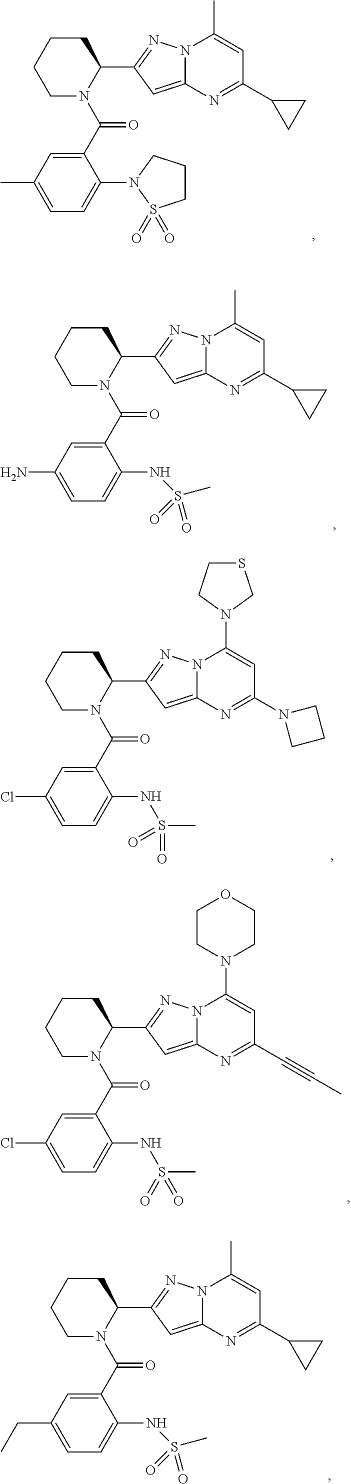

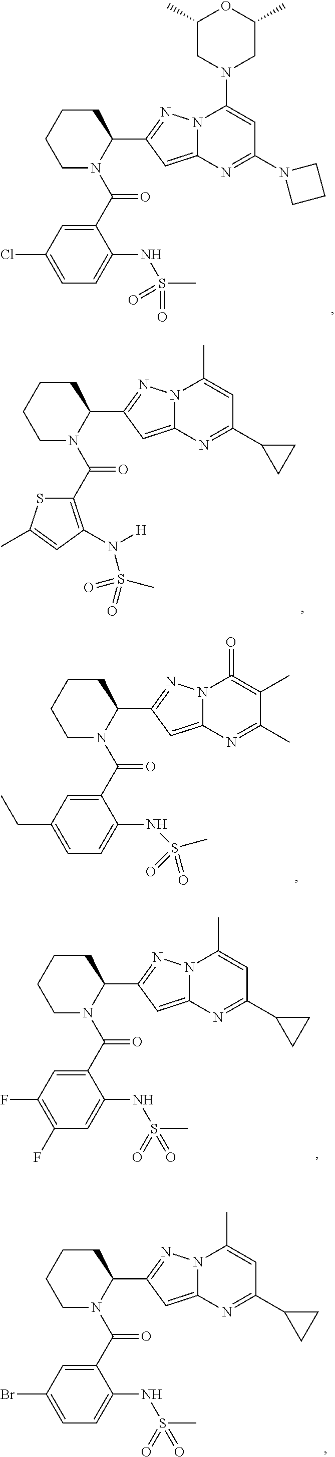

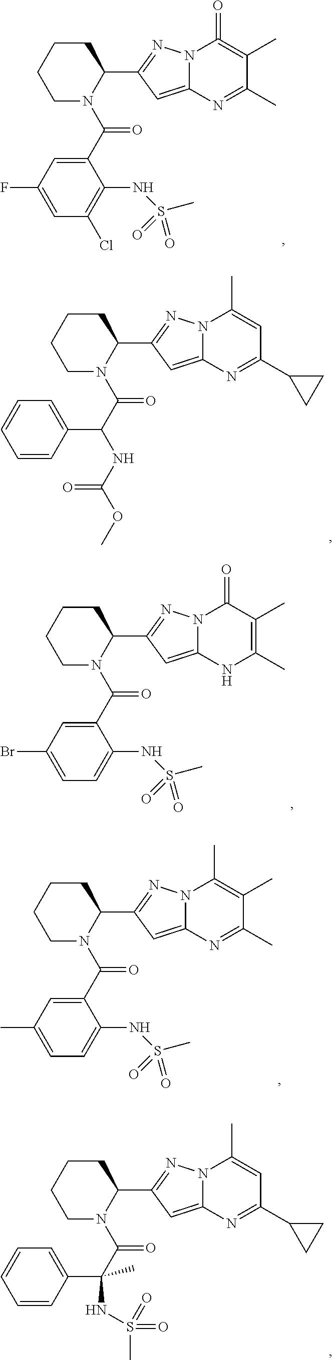

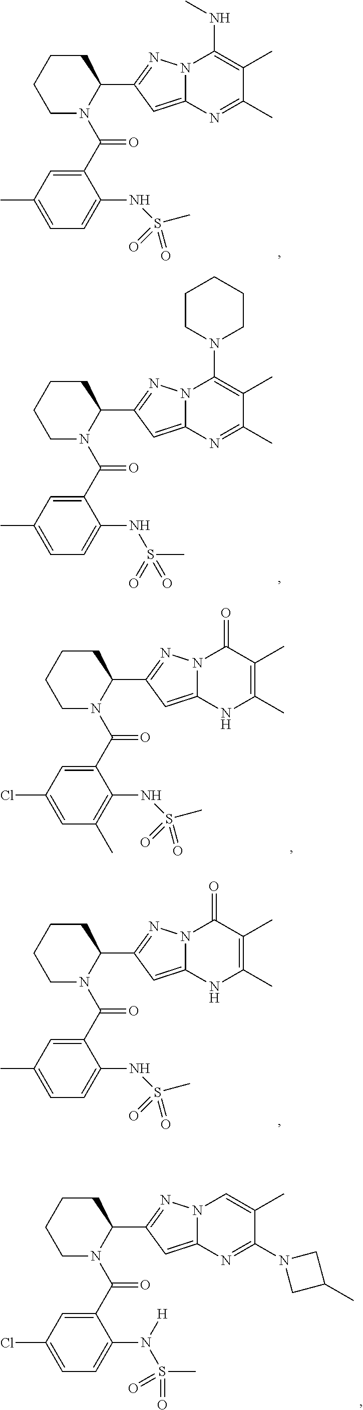

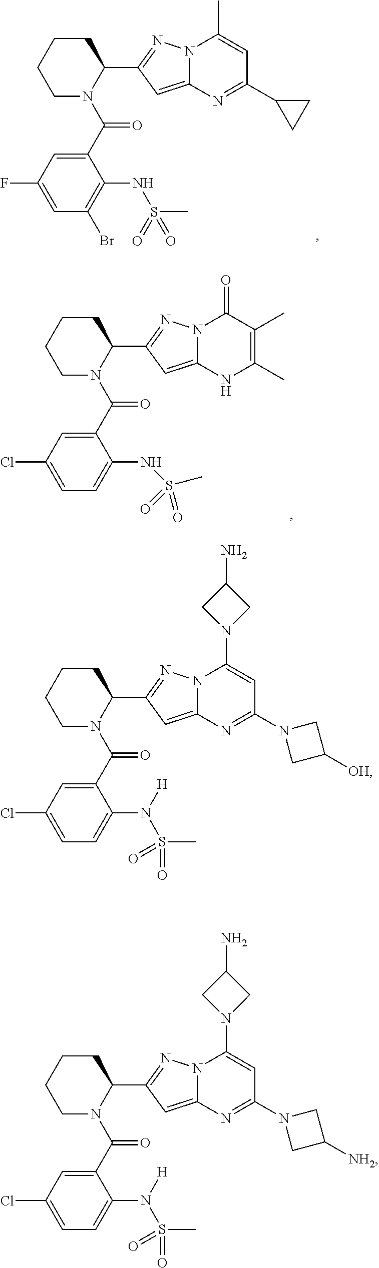 [SMILES: Cc1cc(C2CC2)nc2cc([C@@H]3CCCCN3C(=O)c3cc(F)cc(Br)c3NS(C)(=O)=O)nn12 ; Cc1[nH]c2cc([C@@H]3CCCCN3C(=O)c3cc(Cl)ccc3NS(C)(=O)=O)nn2c(=O)c1C ; CS(=O)(=O)Nc1ccc(Cl)cc1C(=O)N1CCCC[C@H]1c1cc2nc(N3CC(O)C3)cc(N3CC(N)C3)n2n1 ; CS(=O)(=O)Nc1ccc(Cl)cc1C(=O)N1CCCC[C@H]1c1cc2nc(N3CC(N)C3)cc(N3CC(N)C3)n2n1]

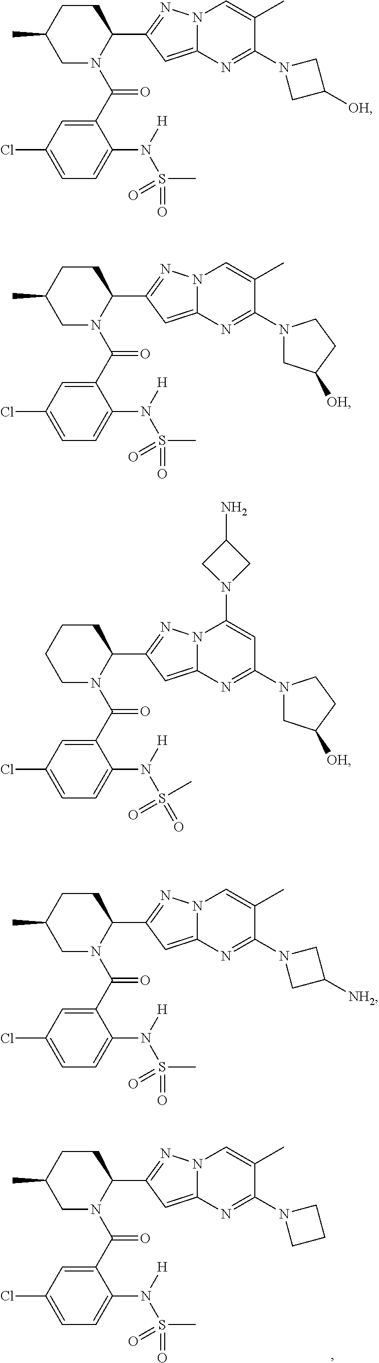 [SMILES: Cc1cn2nc([C@@H]3CC[C@H](C)CN3C(=O)c3cc(Cl)ccc3NS(C)(=O)=O)cc2nc1N1CC(O)C1 ; Cc1cn2nc([C@@H]3CC[C@H](C)CN3C(=O)c3cc(Cl)ccc3NS(C)(=O)=O)cc2nc1N1CC[C@@H](O)C1 ; CS(=O)(=O)Nc1ccc(Cl)cc1C(=O)N1CCCC[C@H]1c1cc2nc(N3CC[C@@H](O)C3)cc(N3CC(N)C3)n2n1 ; Cc1cn2nc([C@@H]3CC[C@H](C)CN3C(=O)c3cc(Cl)ccc3NS(C)(=O)=O)cc2nc1N1CC(N)C1 ; Cc1cn2nc([C@@H]3CC[C@H](C)CN3C(=O)c3cc(Cl)ccc3NS(C)(=O)=O)cc2nc1N1CCC1]

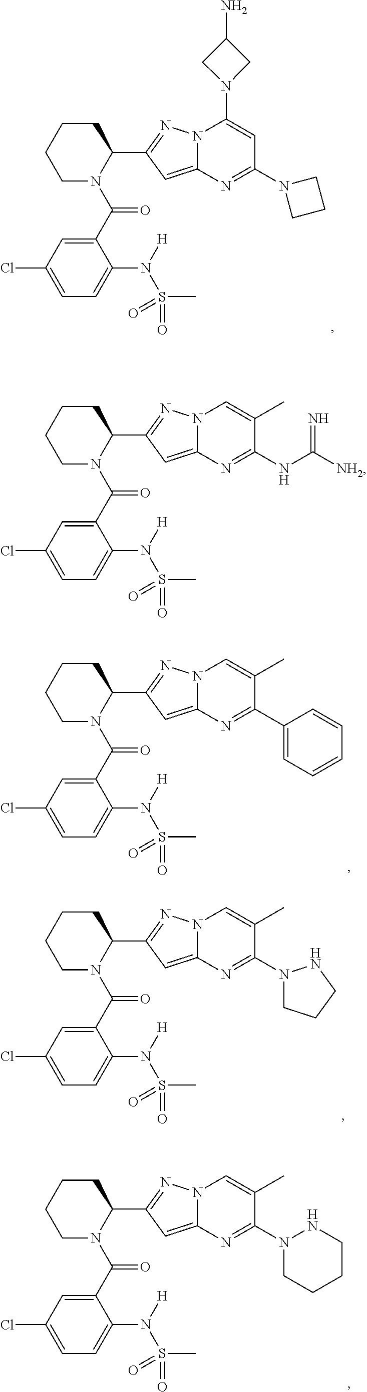

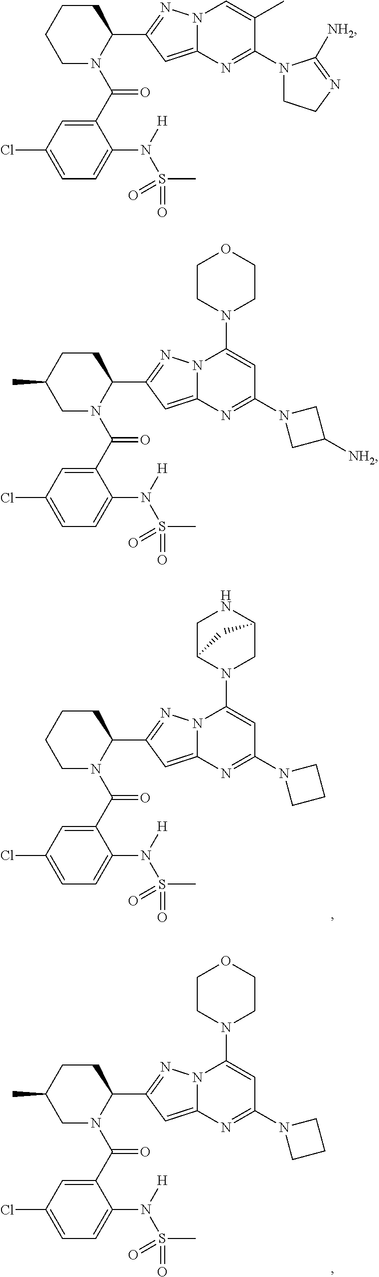 [SMILES: Cc1cn2nc([C@@H]3CCCCN3C(=O)c3cc(Cl)ccc3NS(C)(=O)=O)cc2nc1N1CCN=C1N ; C[C@H]1CC[C@@H](c2cc3nc(N4CC(N)C4)cc(N4CCOCC4)n3n2)N(C(=O)c2cc(Cl)ccc2NS(C)(=O)=O)C1 ; CS(=O)(=O)Nc1ccc(Cl)cc1C(=O)N1CCCC[C@H]1c1cc2nc(N3CCC3)cc(N3C[C@H]4C[C@@H]3CN4)n2n1 ; C[C@H]1CC[C@@H](c2cc3nc(N4CCC4)cc(N4CCOCC4)n3n2)N(C(=O)c2cc(Cl)ccc2NS(C)(=O)=O)C1]

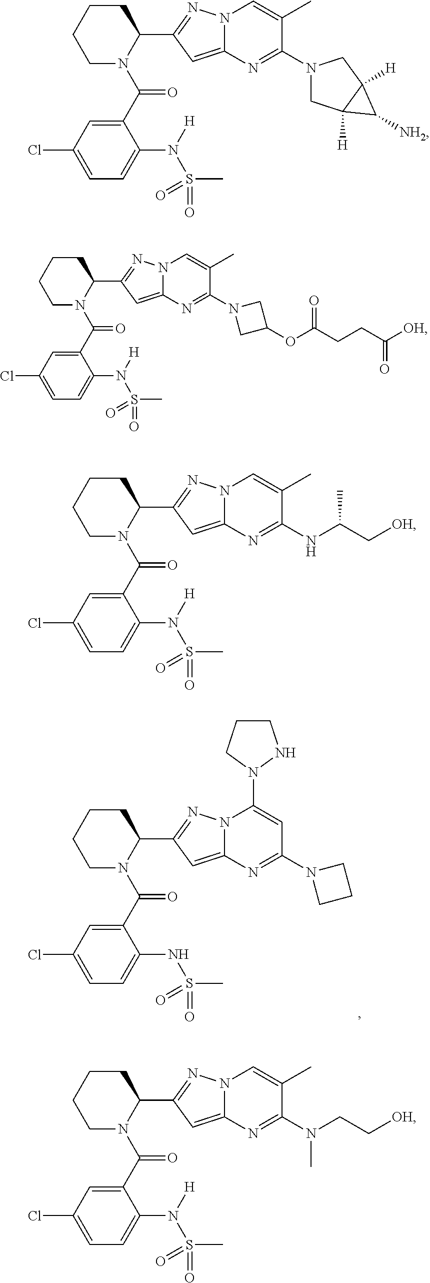

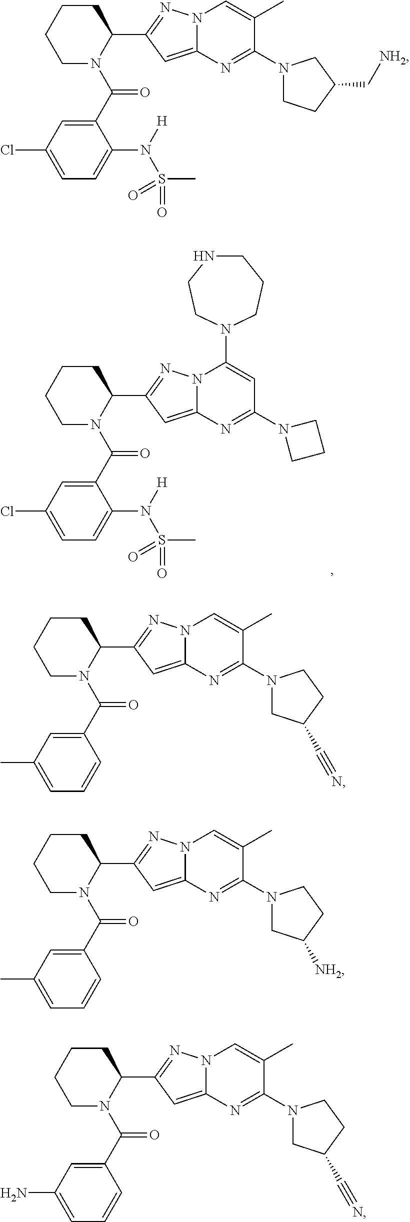

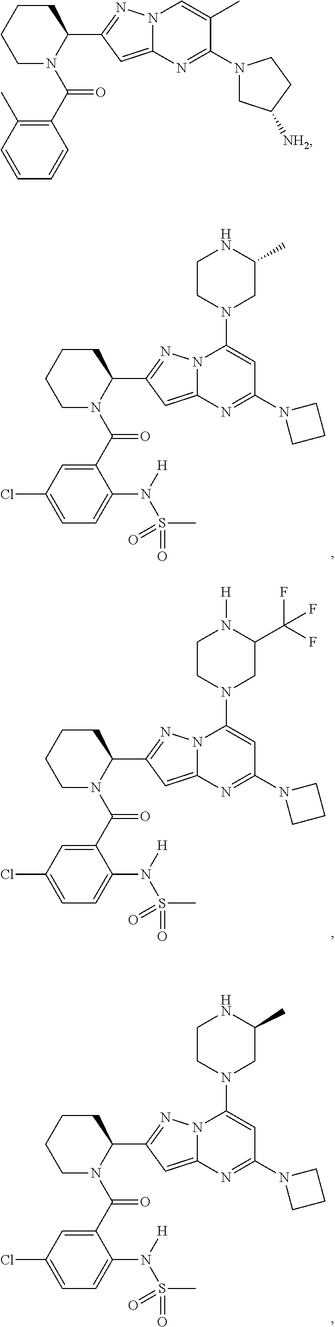 [SMILES: Cc1ccccc1C(=O)N1CCCC[C@H]1c1cc2nc(N3CC[C@H](N)C3)c(C)cn2n1 ; C[C@@H]1CN(c2cc(N3CCC3)nc3cc([C@@H]4CCCCN4C(=O)c4cc(Cl)ccc4NS(C)(=O)=O)nn23)CCN1 ; CS(=O)(=O)Nc1ccc(Cl)cc1C(=O)N1CCCC[C@H]1c1cc2nc(N3CCC3)cc(N3CCNC(C(F)(F)F)C3)n2n1 ; C[C@H]1CN(c2cc(N3CCC3)nc3cc([C@@H]4CCCCN4C(=O)c4cc(Cl)ccc4NS(C)(=O)=O)nn23)CCN1]

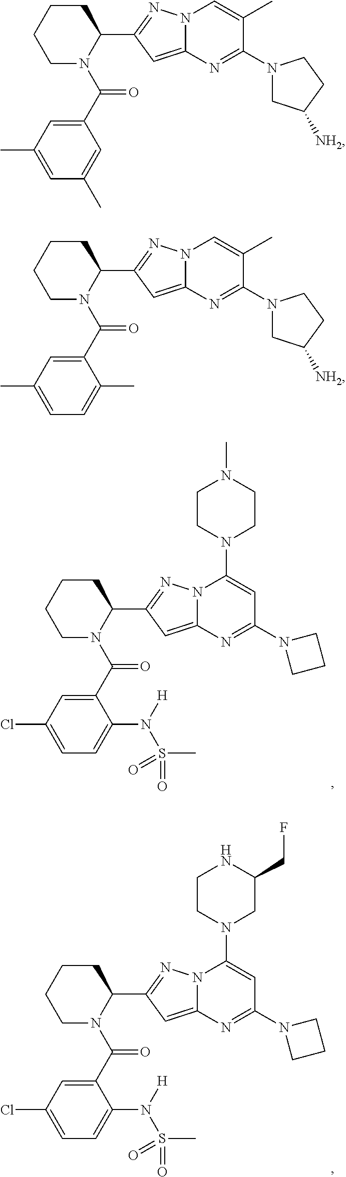

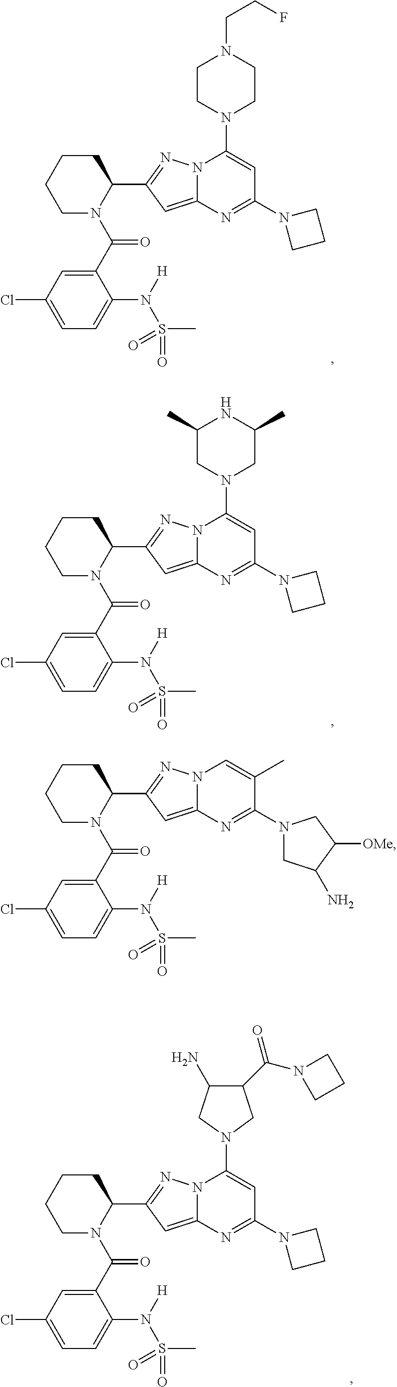

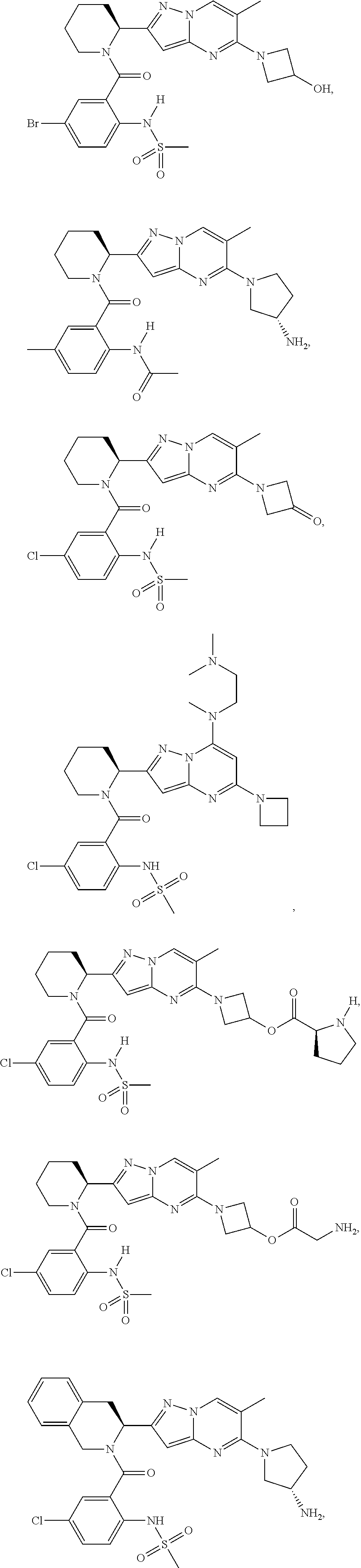

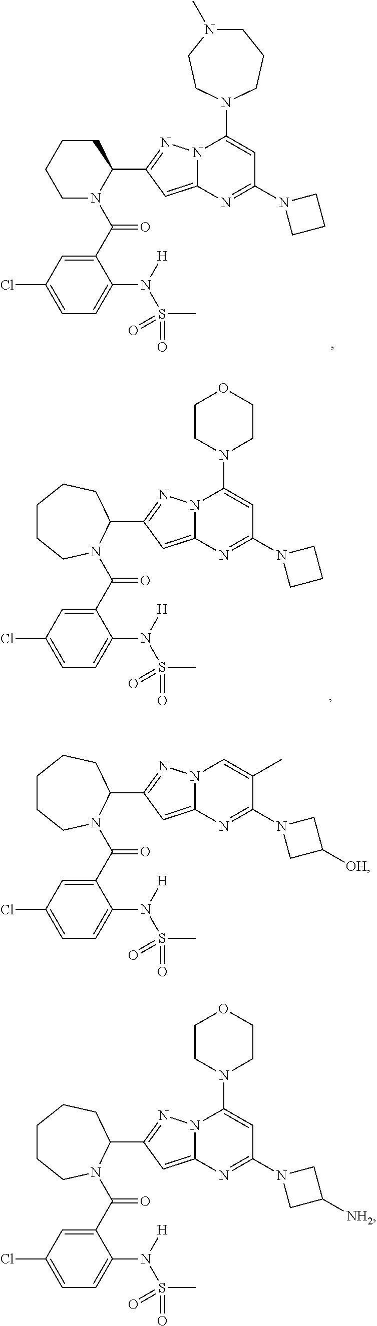 [SMILES: CN1CCCN(c2cc(N3CCC3)nc3cc([C@@H]4CCCCN4C(=O)c4cc(Cl)ccc4NS(C)(=O)=O)nn23)CC1 ; CS(=O)(=O)Nc1ccc(Cl)cc1C(=O)N1CCCCCC1c1cc2nc(N3CCC3)cc(N3CCOCC3)n2n1 ; Cc1cn2nc(C3CCCCCN3C(=O)c3cc(Cl)ccc3NS(C)(=O)=O)cc2nc1N1CC(O)C1 ; CS(=O)(=O)Nc1ccc(Cl)cc1C(=O)N1CCCCCC1c1cc2nc(N3CC(N)C3)cc(N3CCOCC3)n2n1]

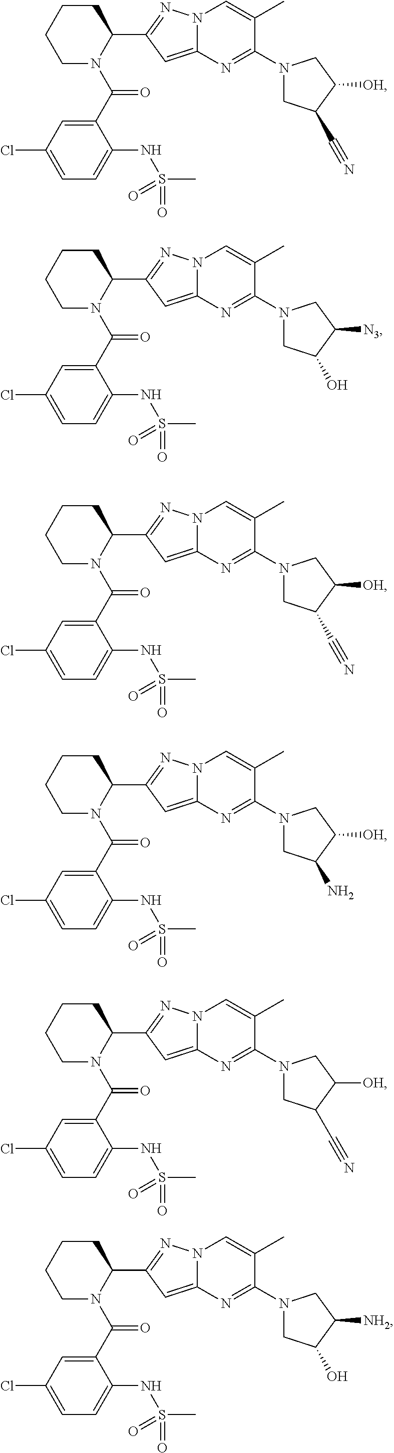

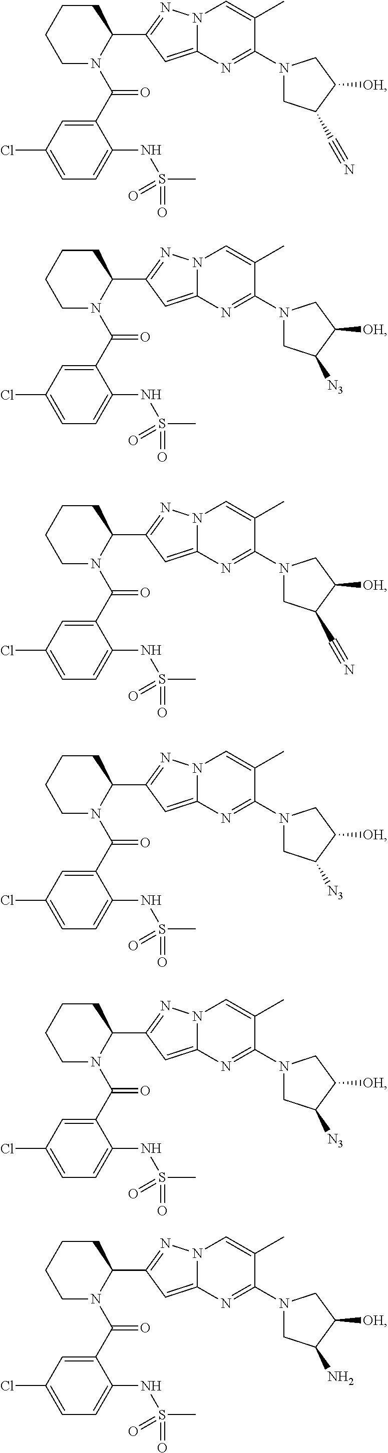

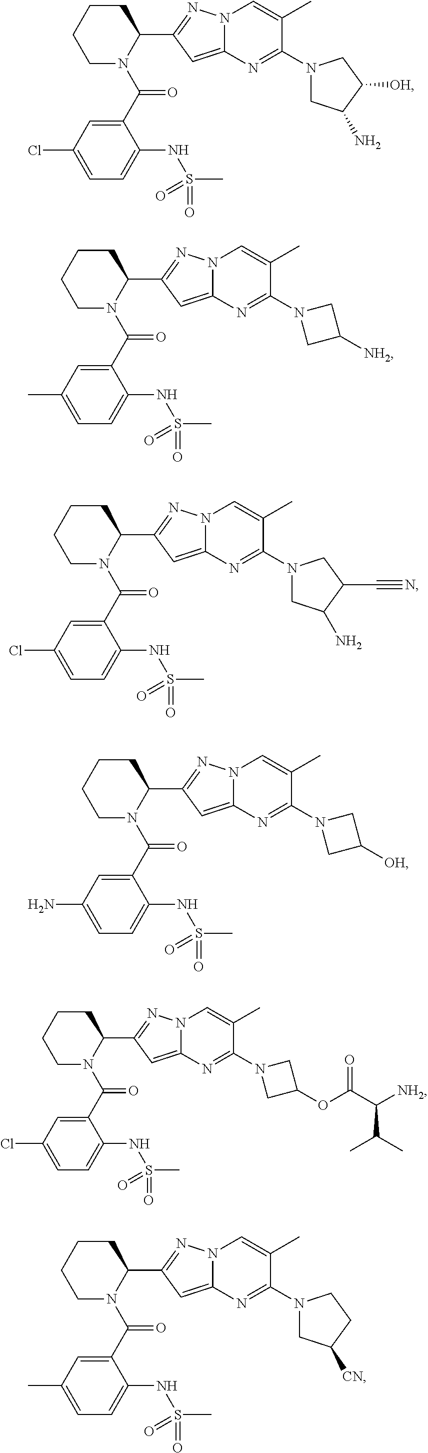

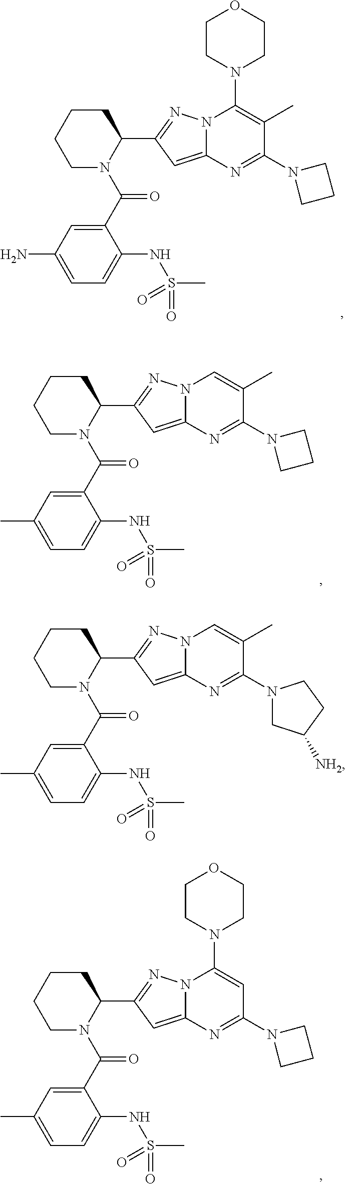 [SMILES: Cc1c(N2CCC2)nc2cc([C@@H]3CCCCN3C(=O)c3cc(N)ccc3NS(C)(=O)=O)nn2c1N1CCOCC1 ; Cc1ccc(NS(C)(=O)=O)c(C(=O)N2CCCC[C@H]2c2cc3nc(N4CCC4)c(C)cn3n2)c1 ; Cc1ccc(NS(C)(=O)=O)c(C(=O)N2CCCC[C@H]2c2cc3nc(N4CC[C@H](N)C4)c(C)cn3n2)c1 ; Cc1ccc(NS(C)(=O)=O)c(C(=O)N2CCCC[C@H]2c2cc3nc(N4CCC4)cc(N4CCOCC4)n3n2)c1]

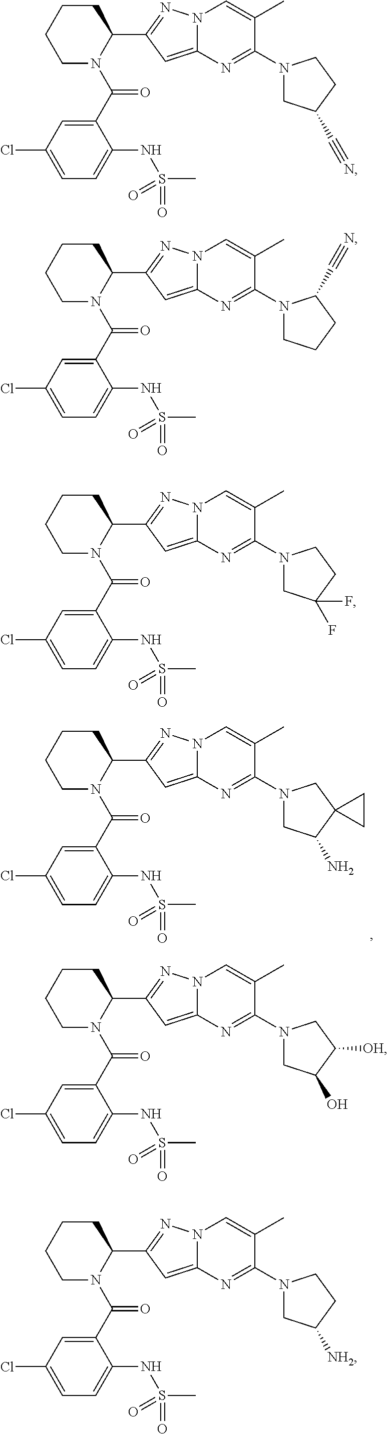

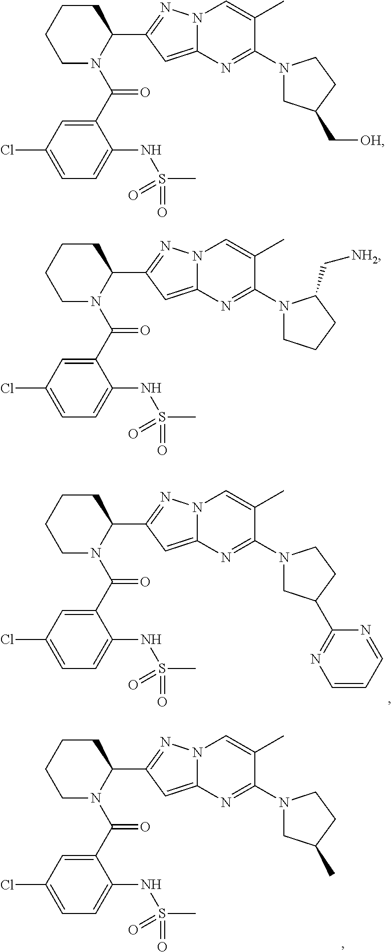

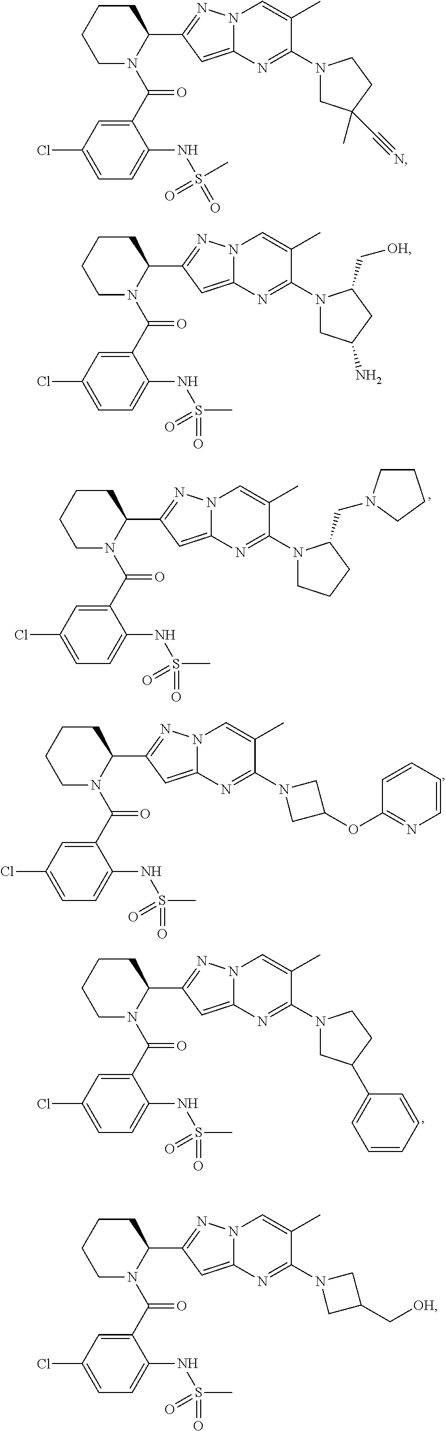

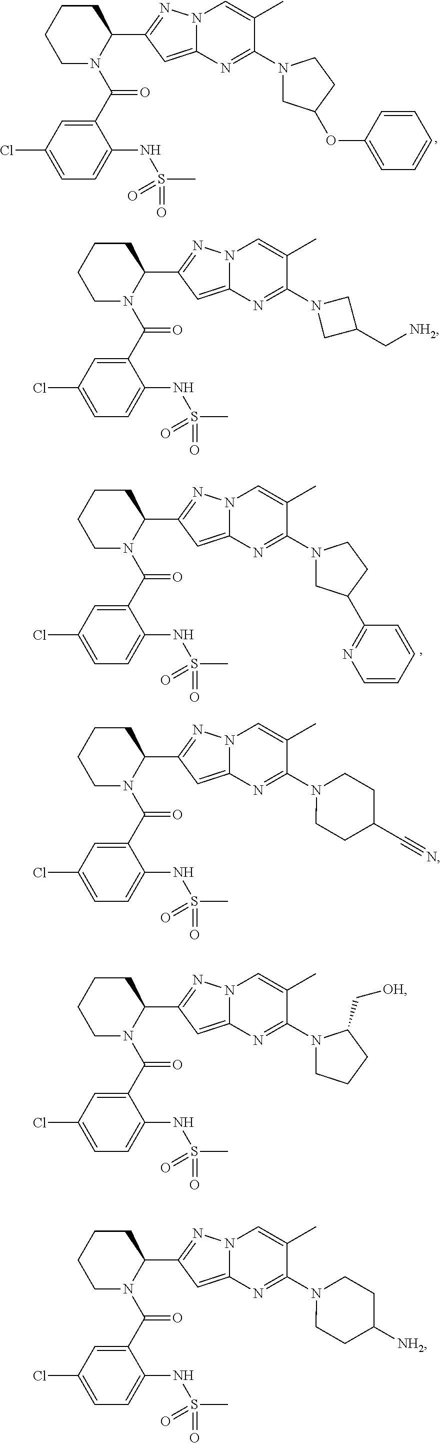

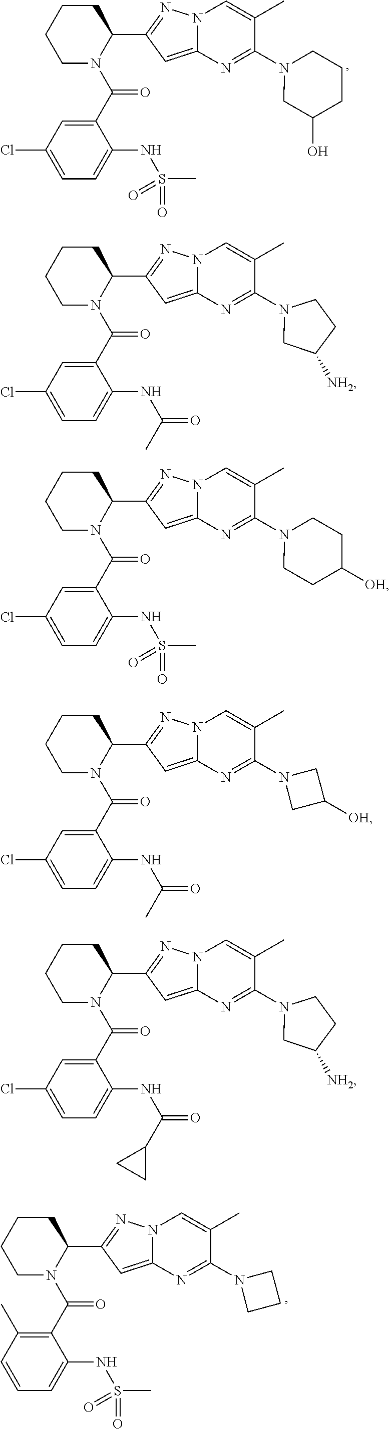

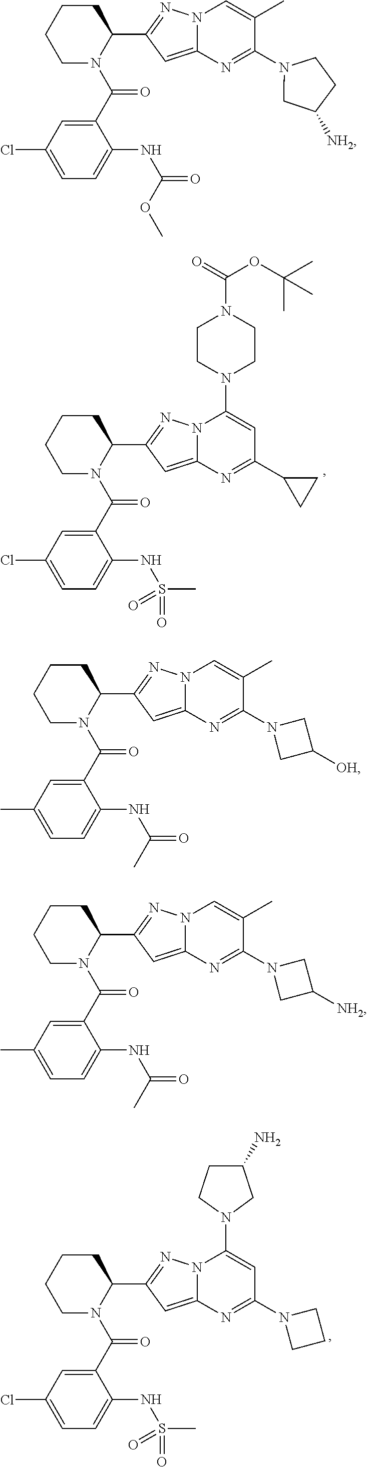

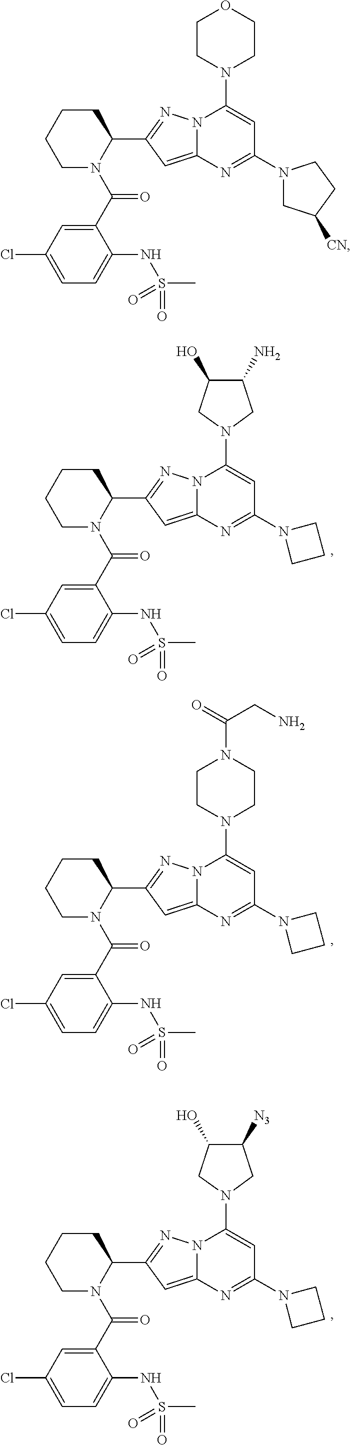

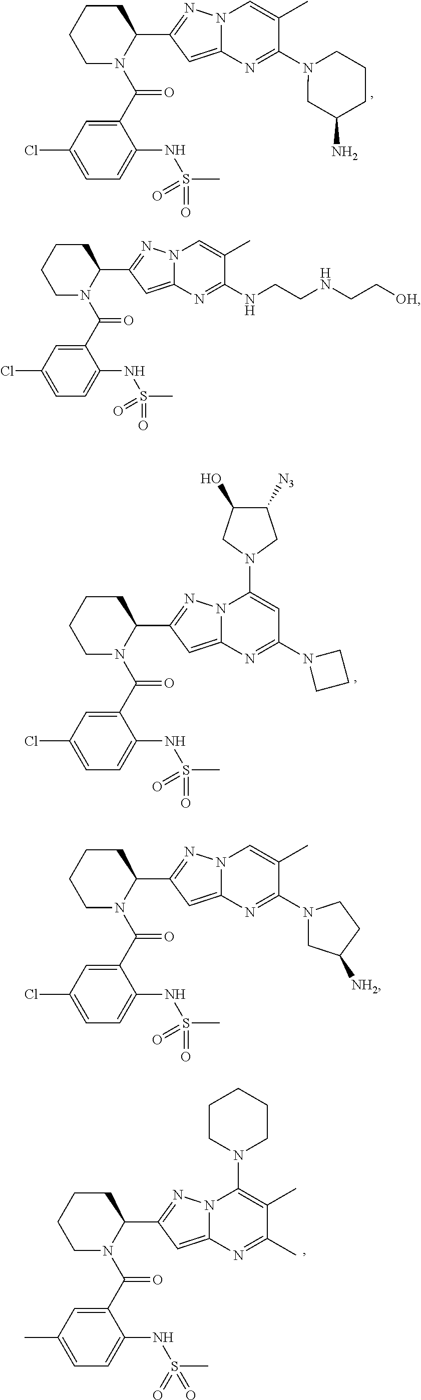

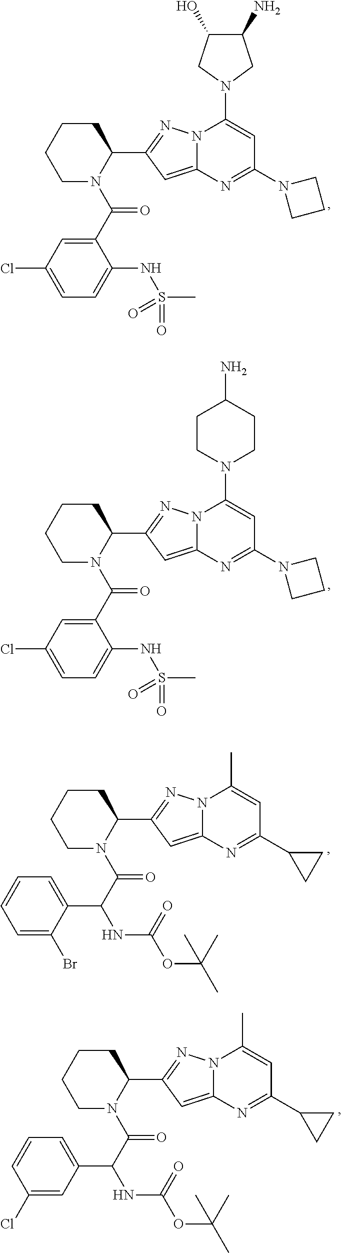

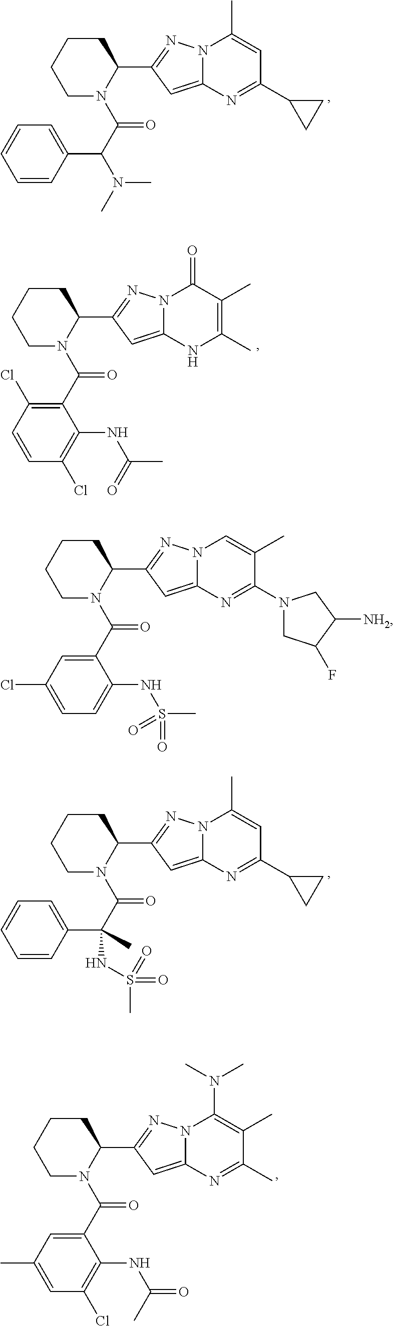

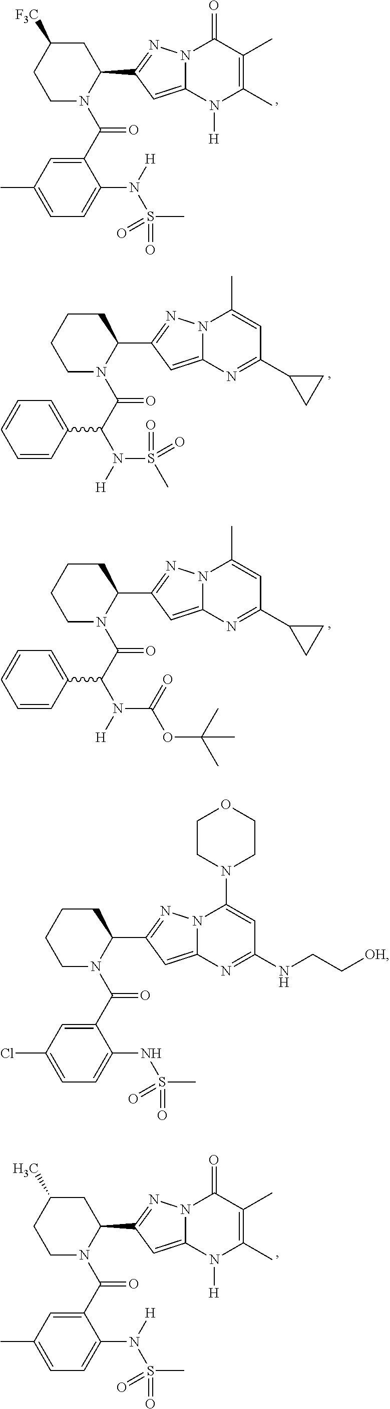

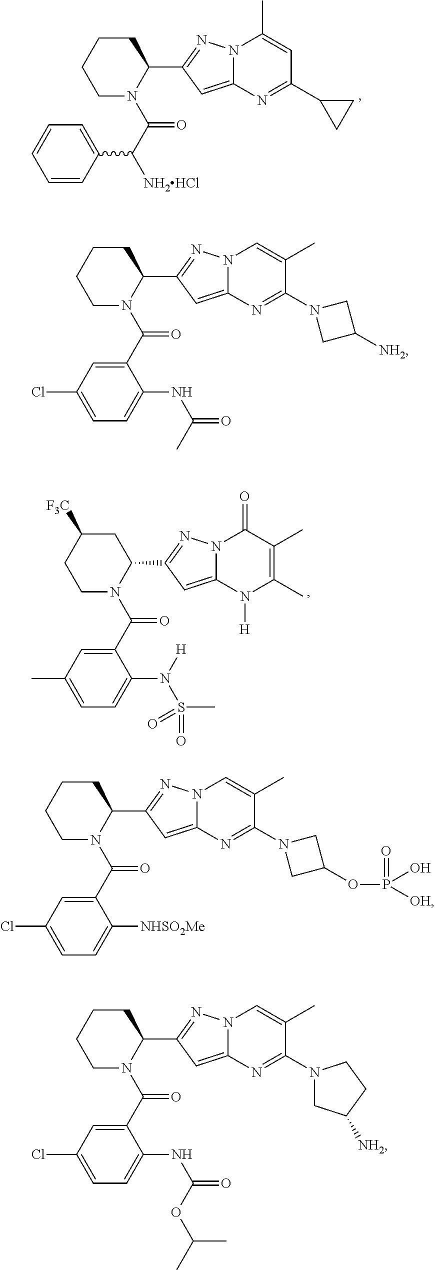

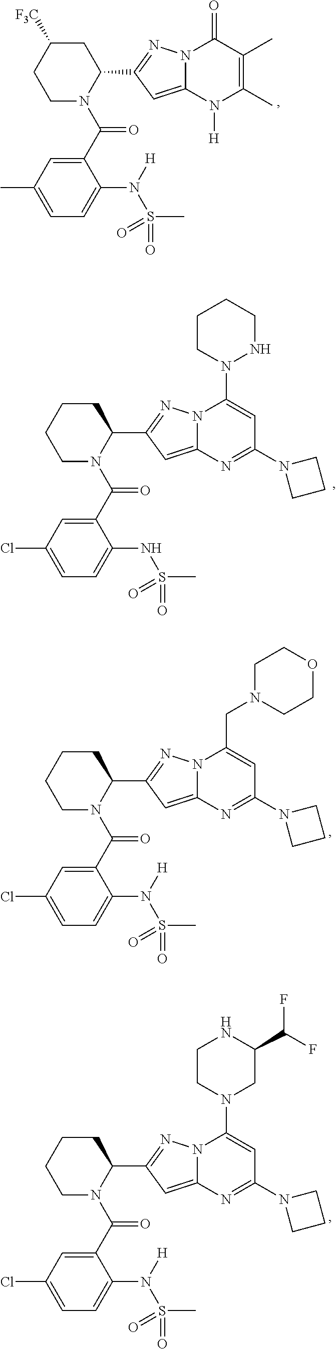

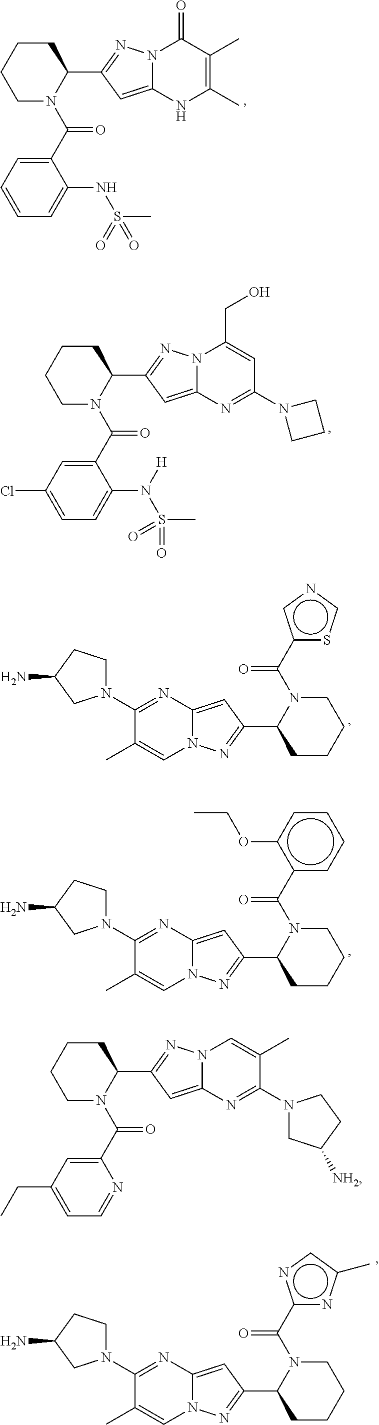

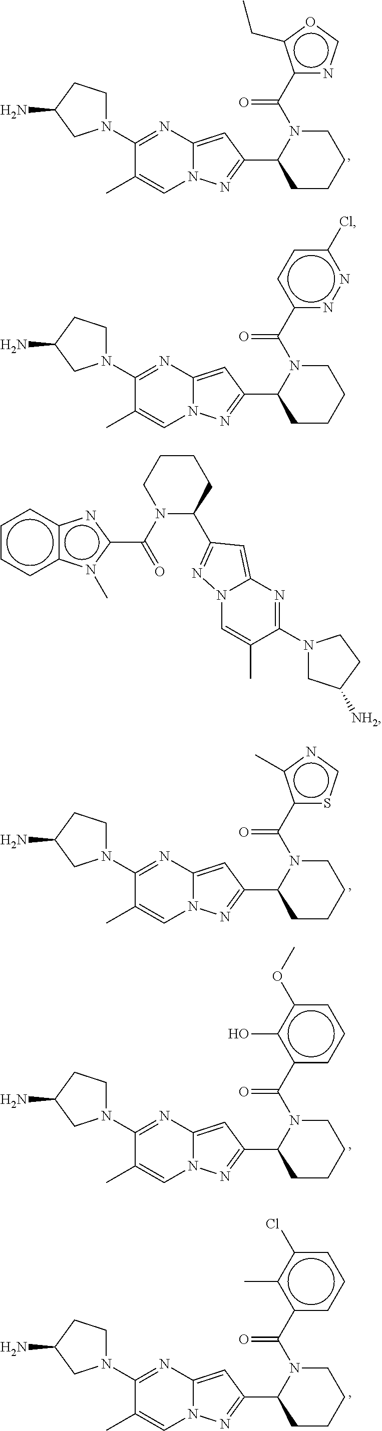

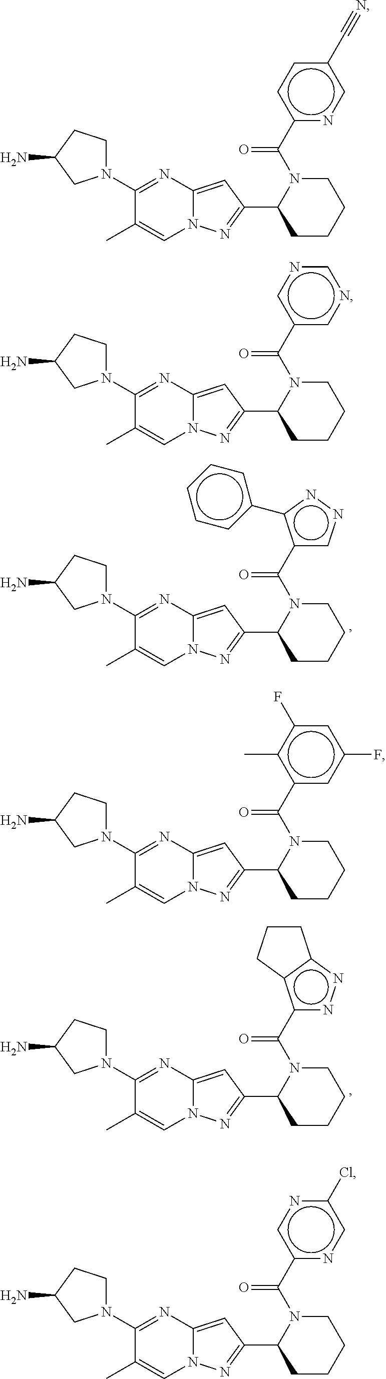

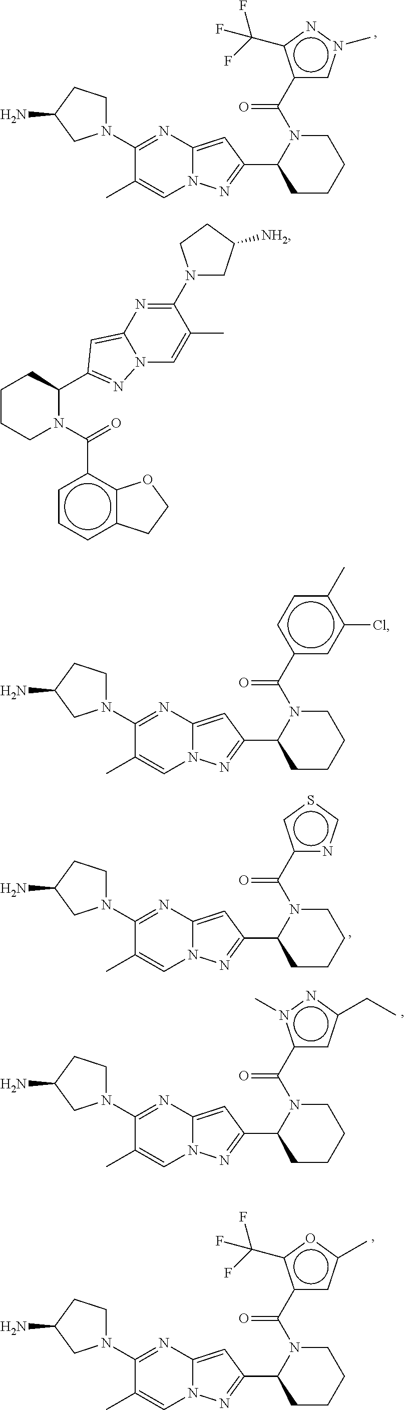

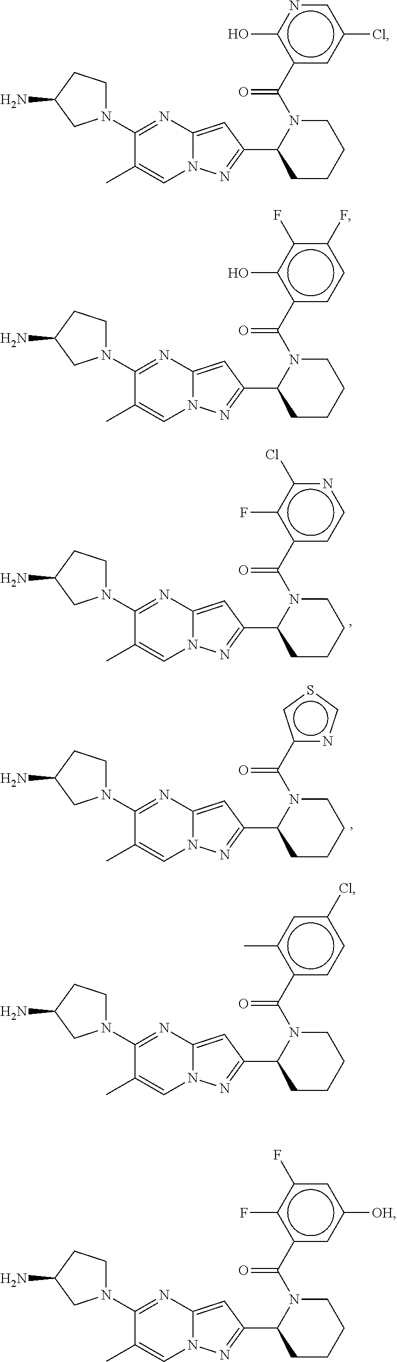

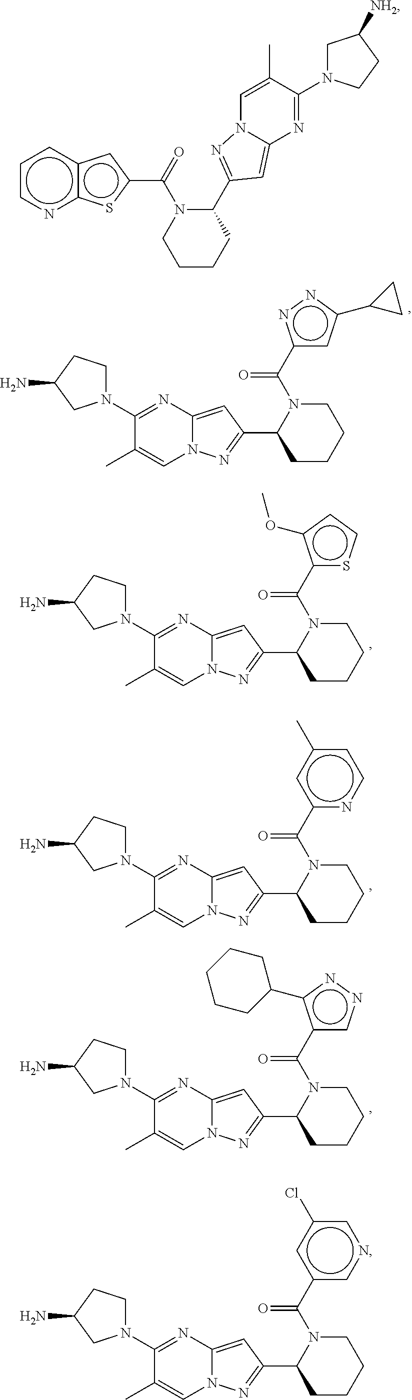

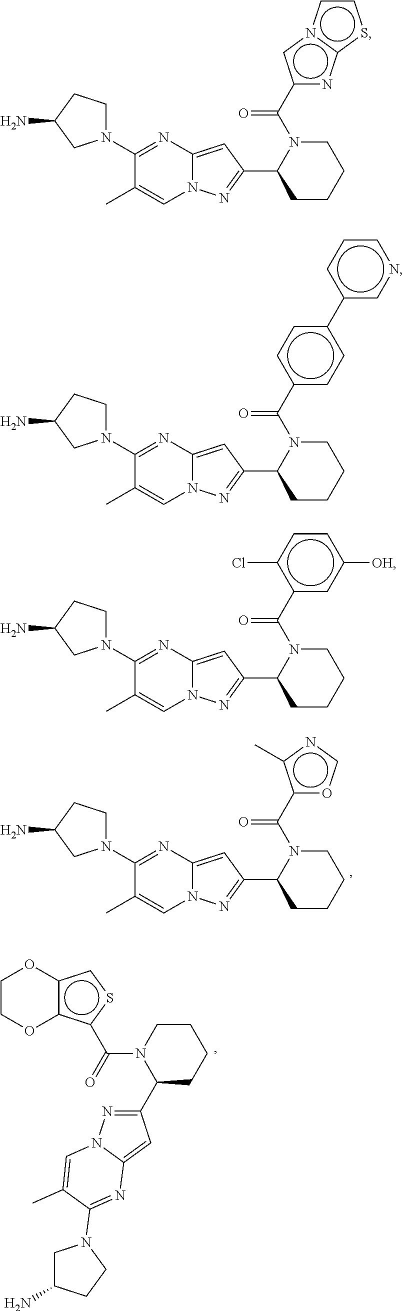

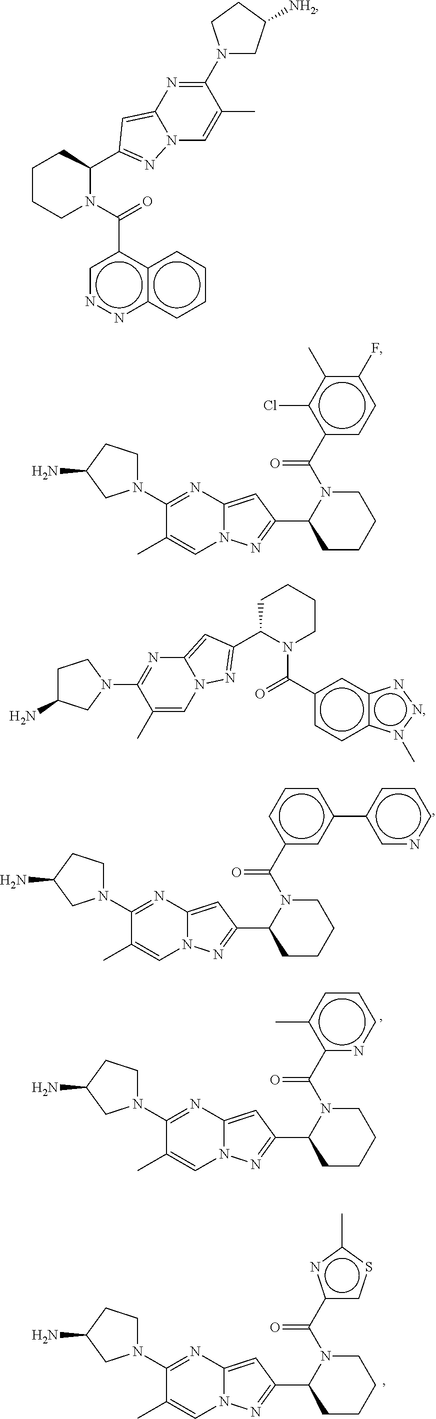

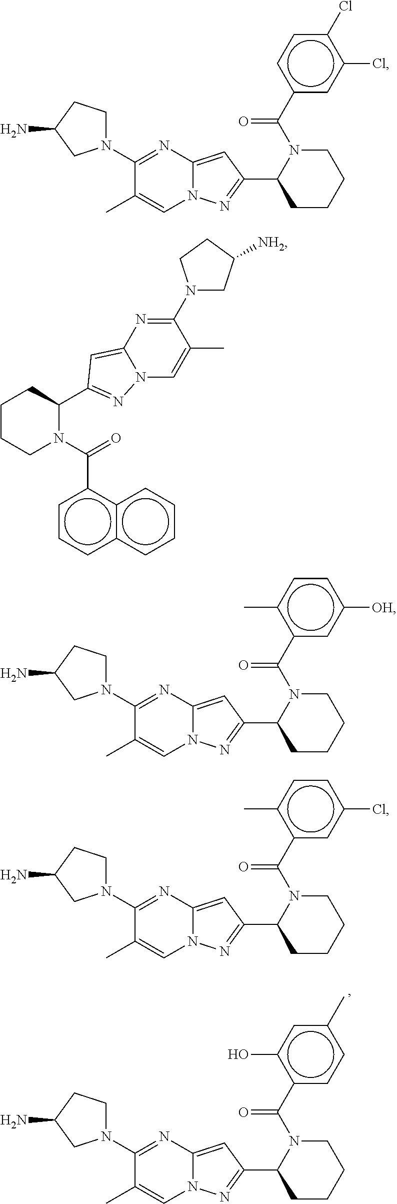

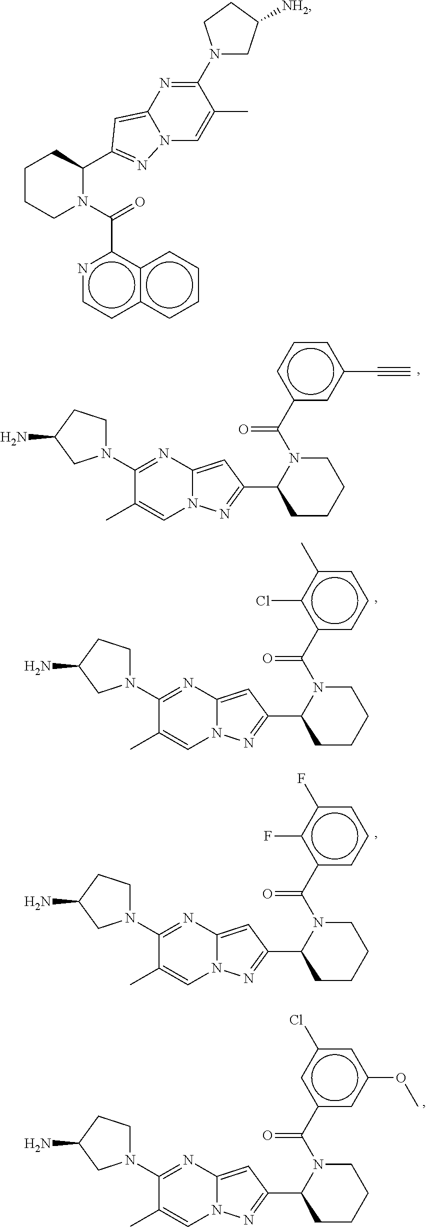

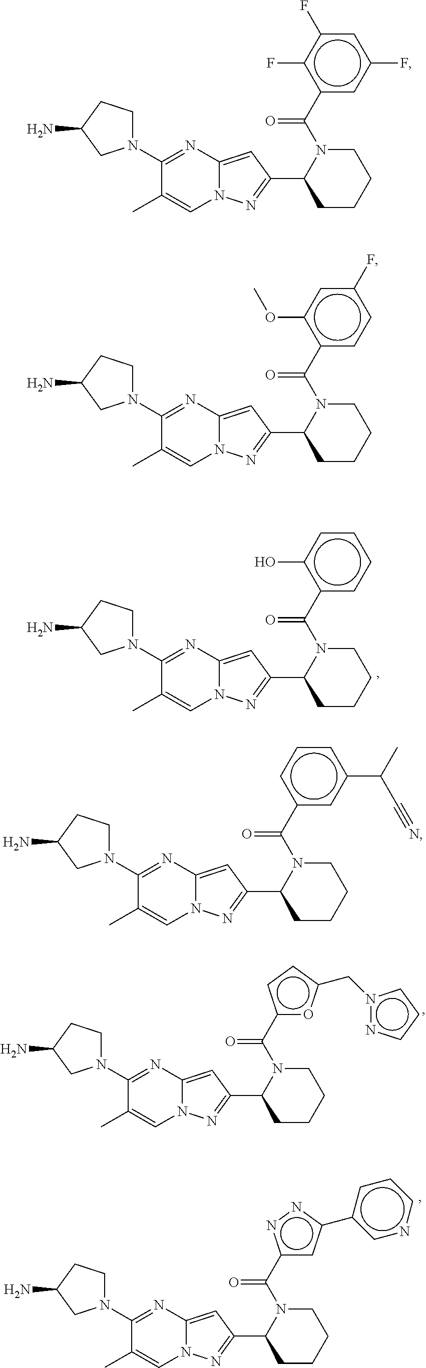

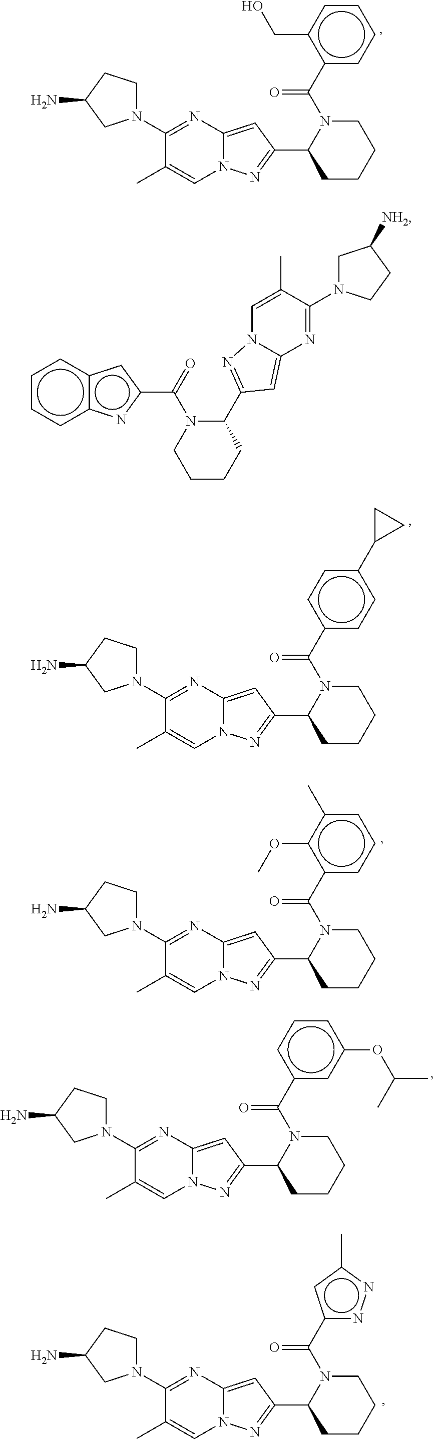

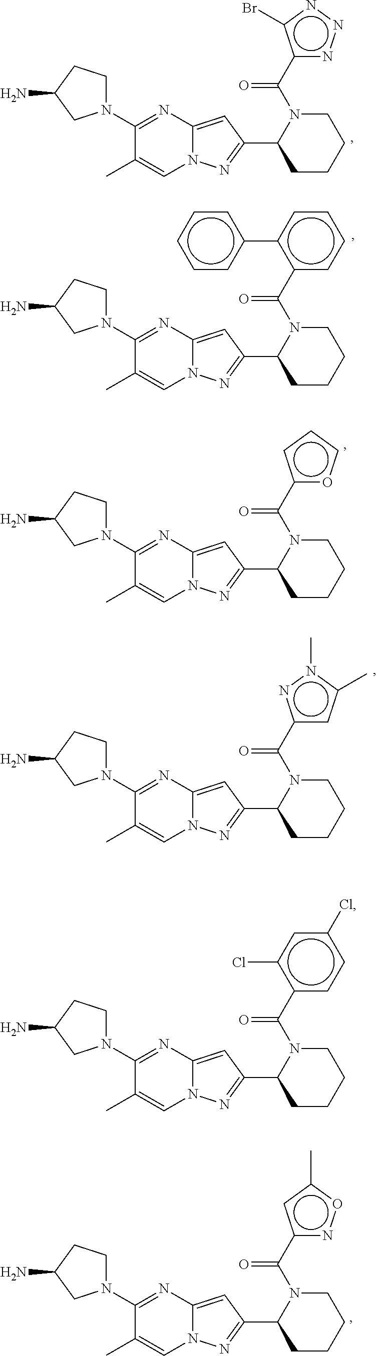

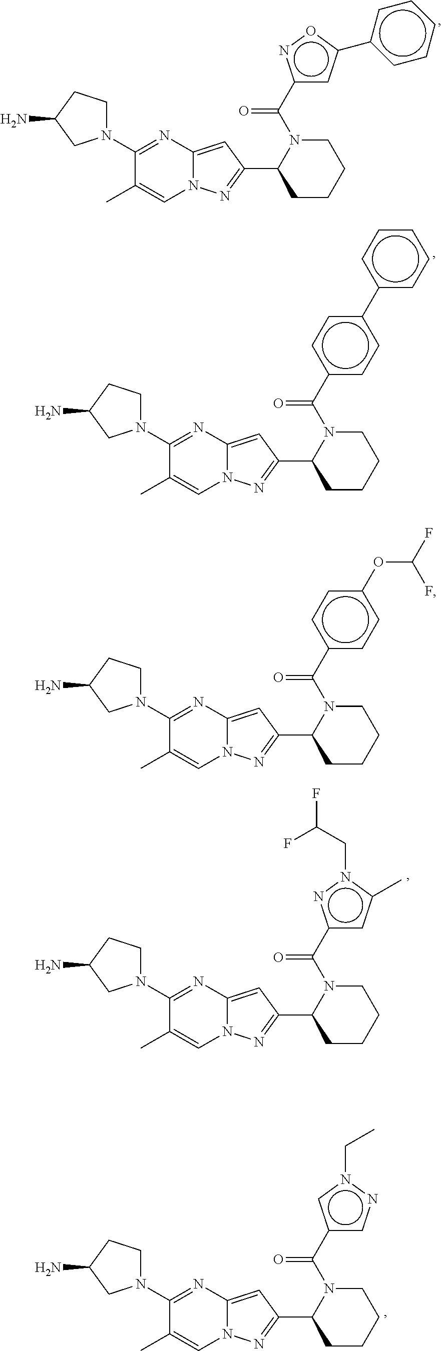

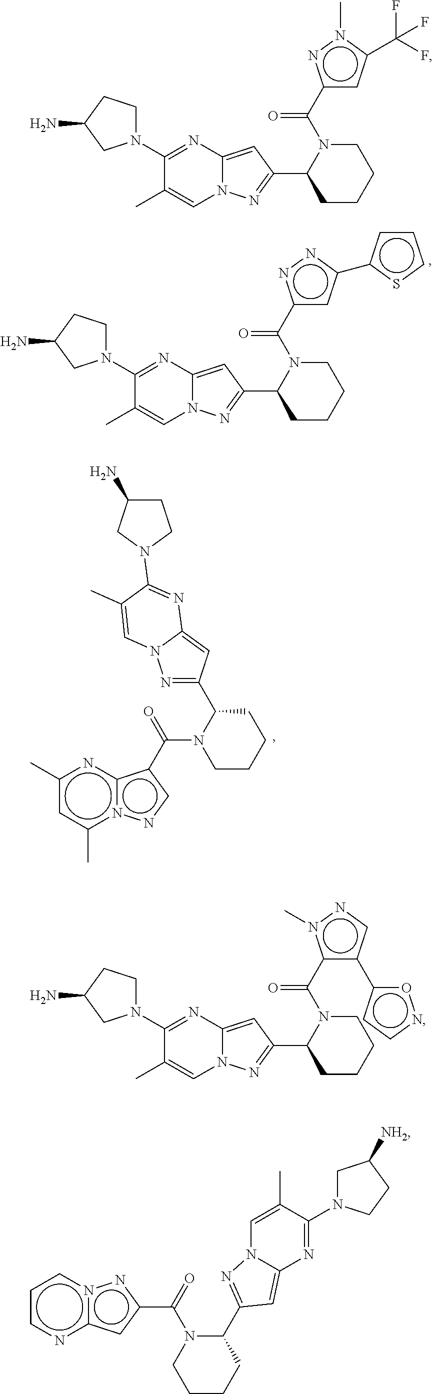

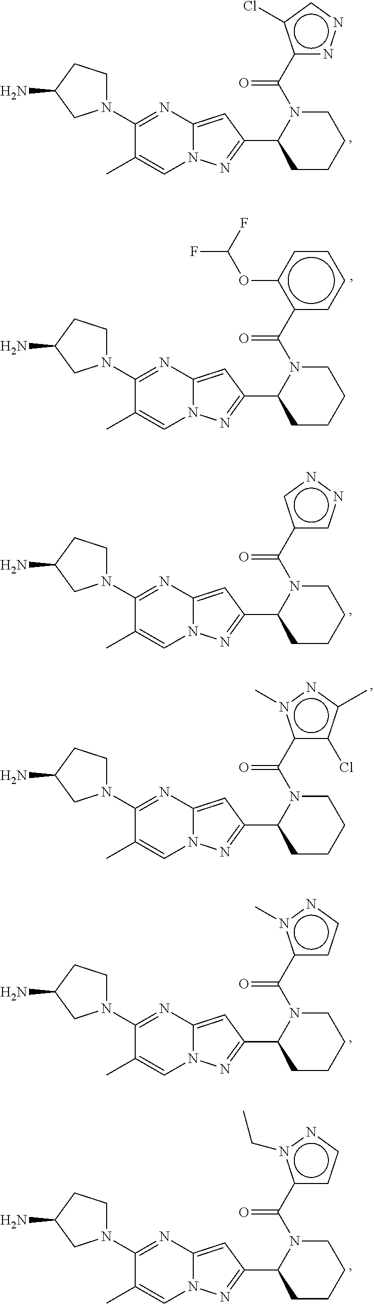

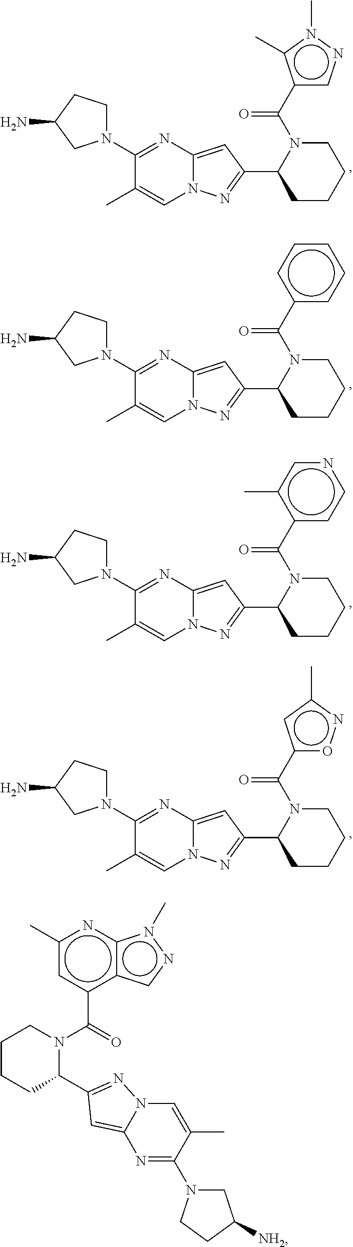

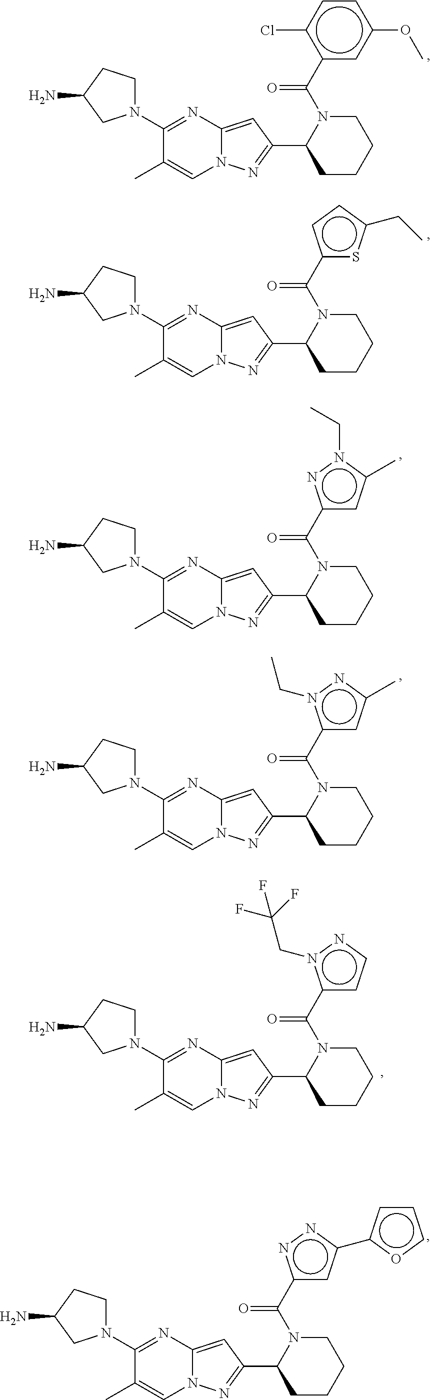

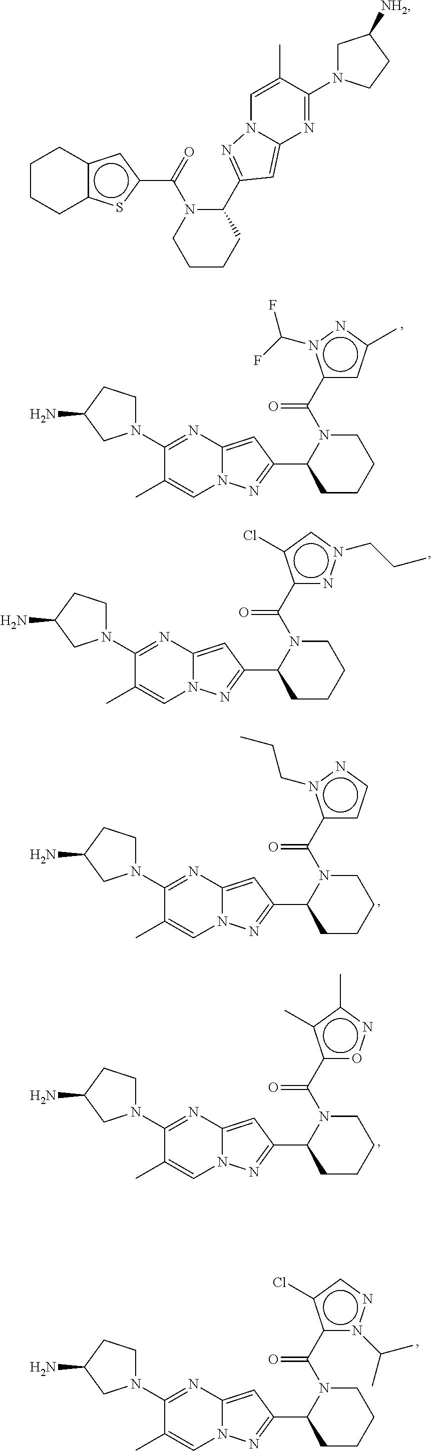

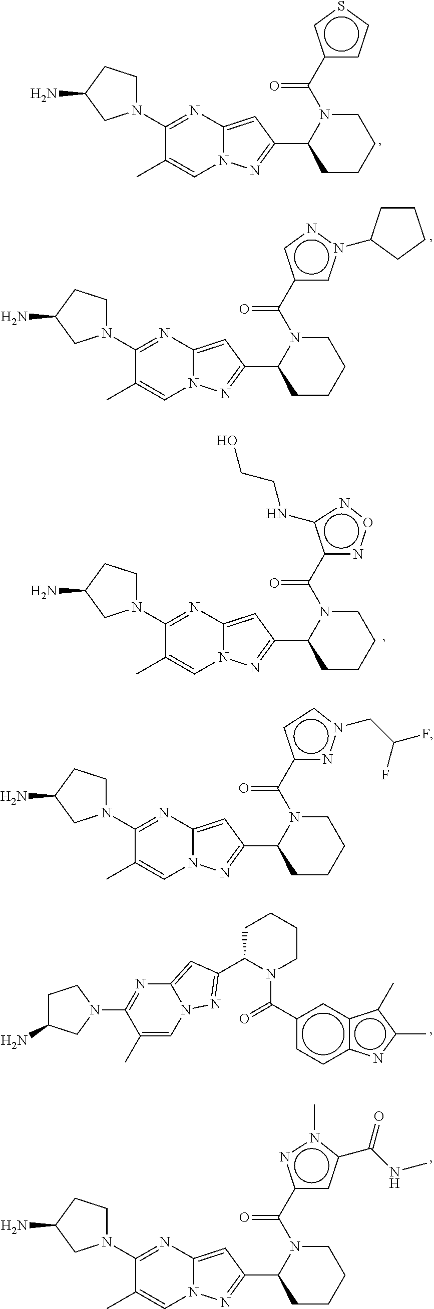

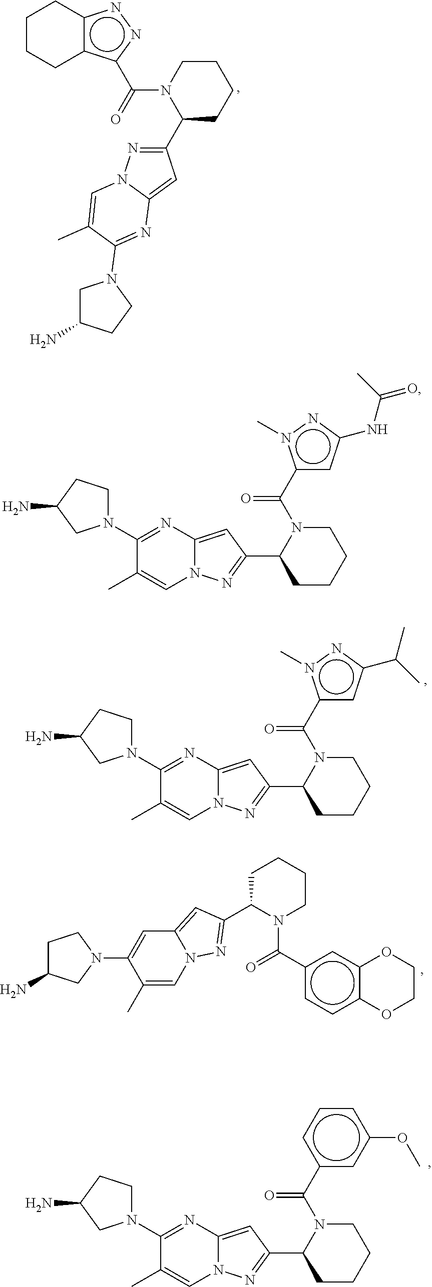

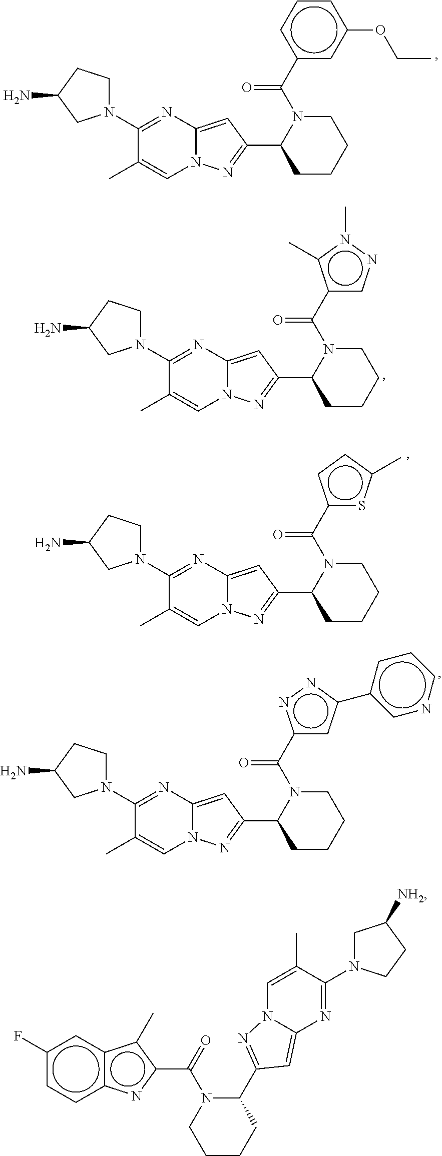

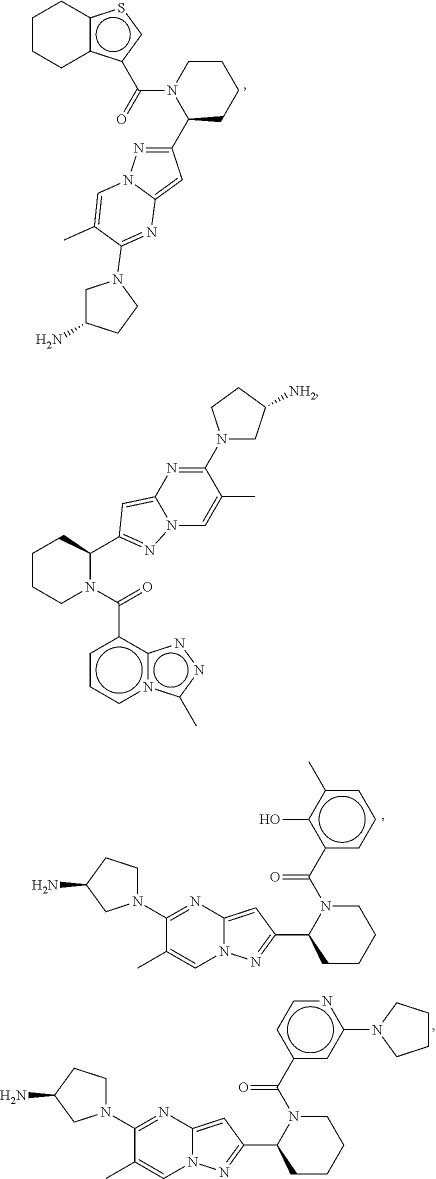

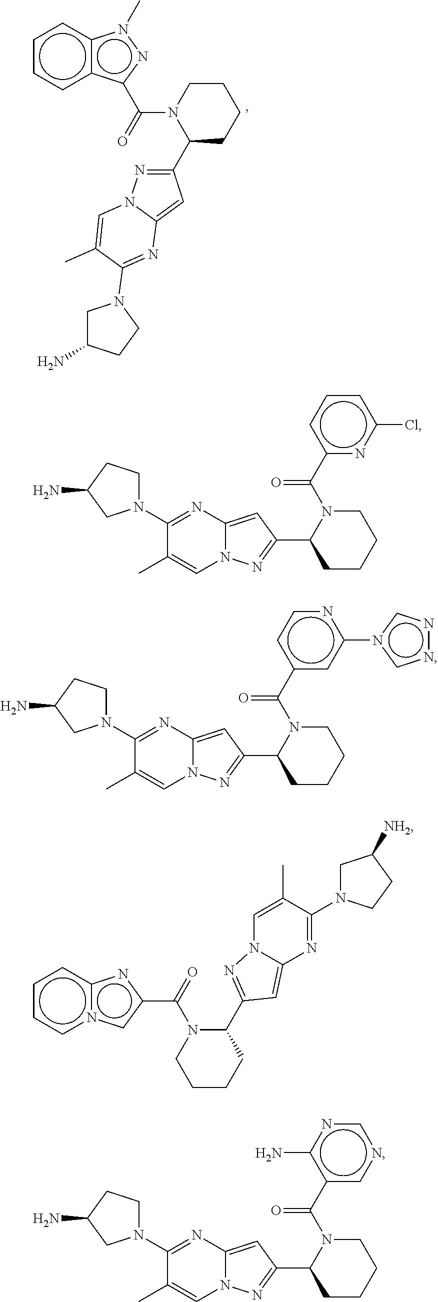

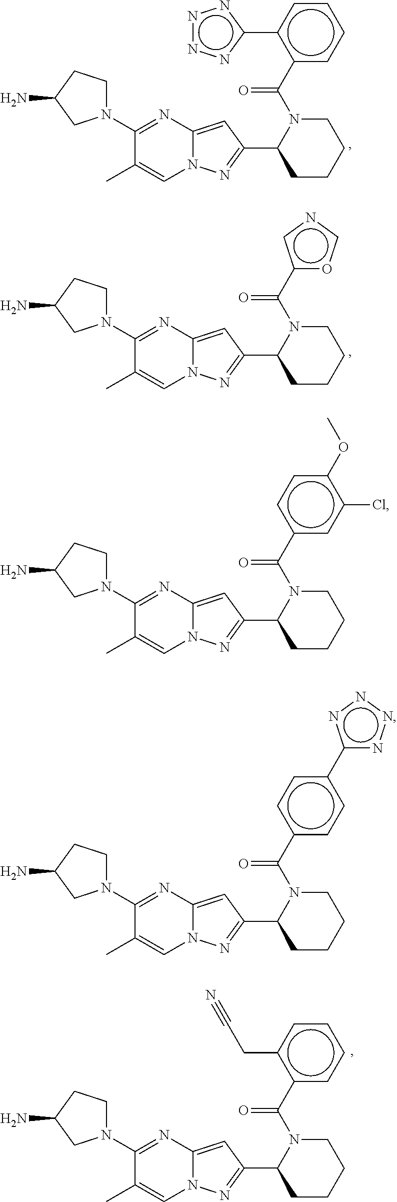

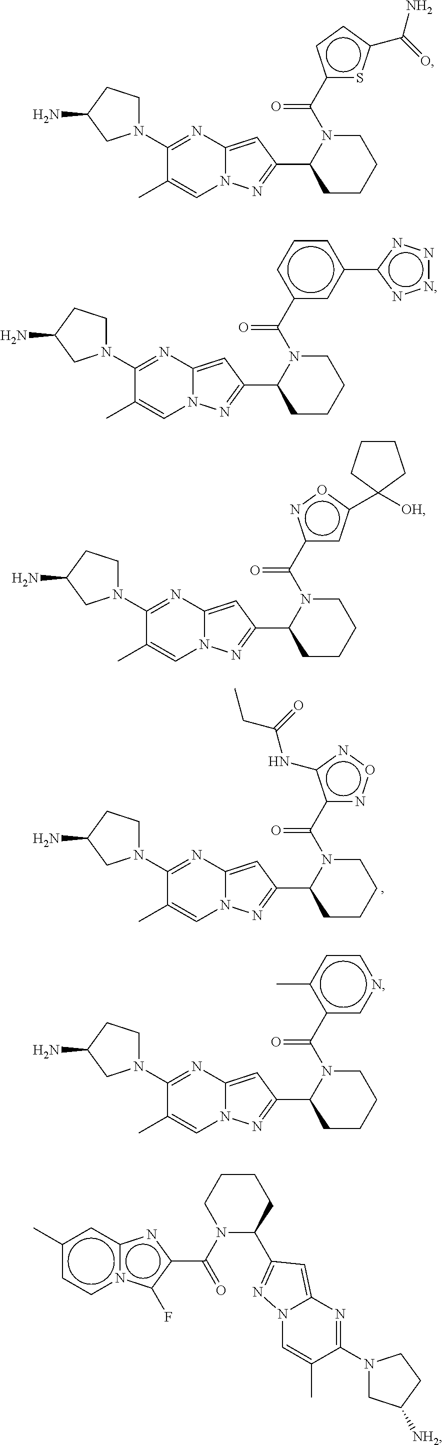

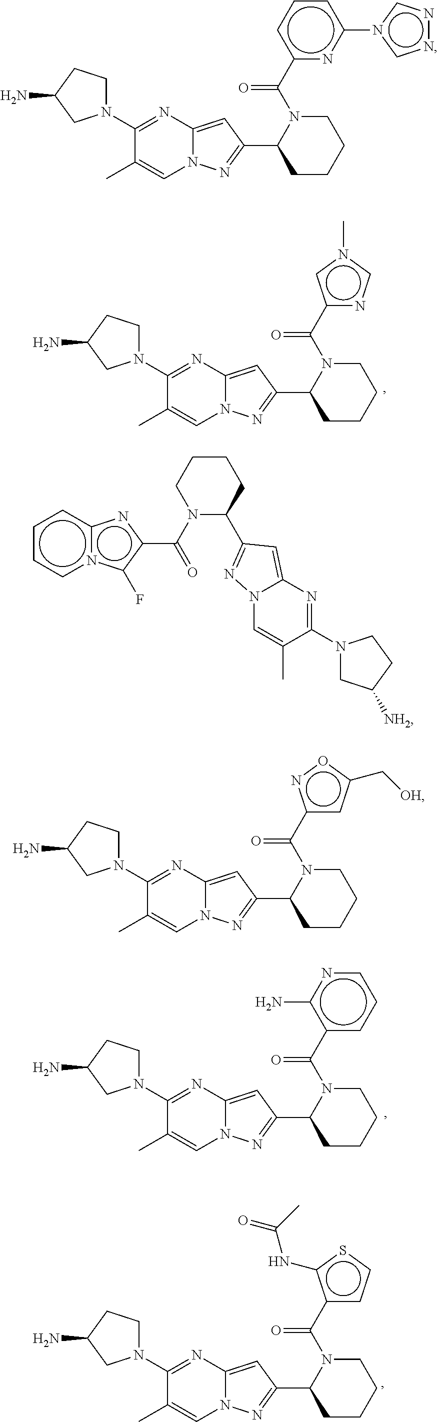

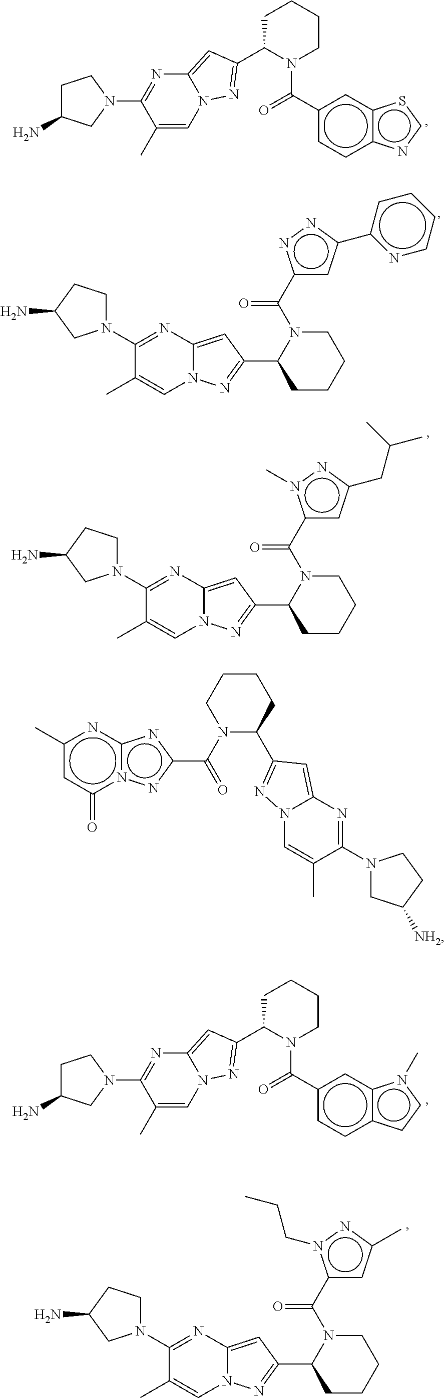

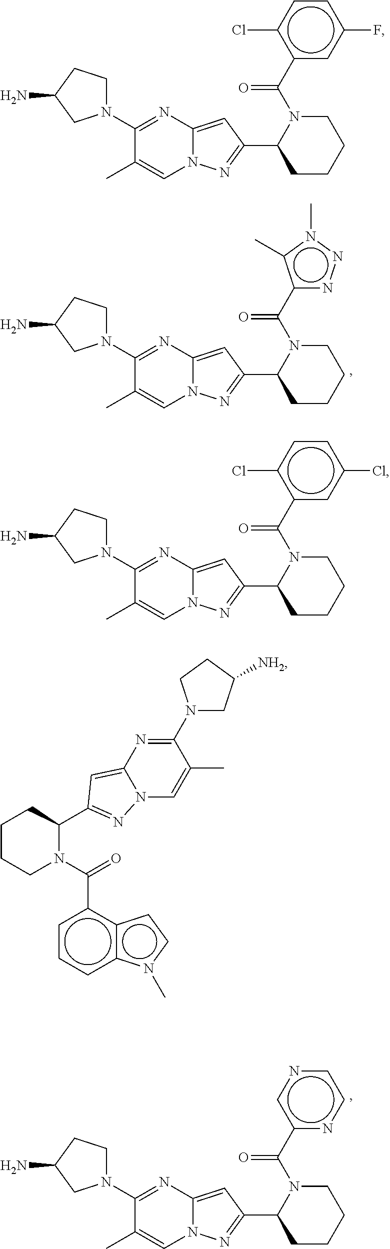

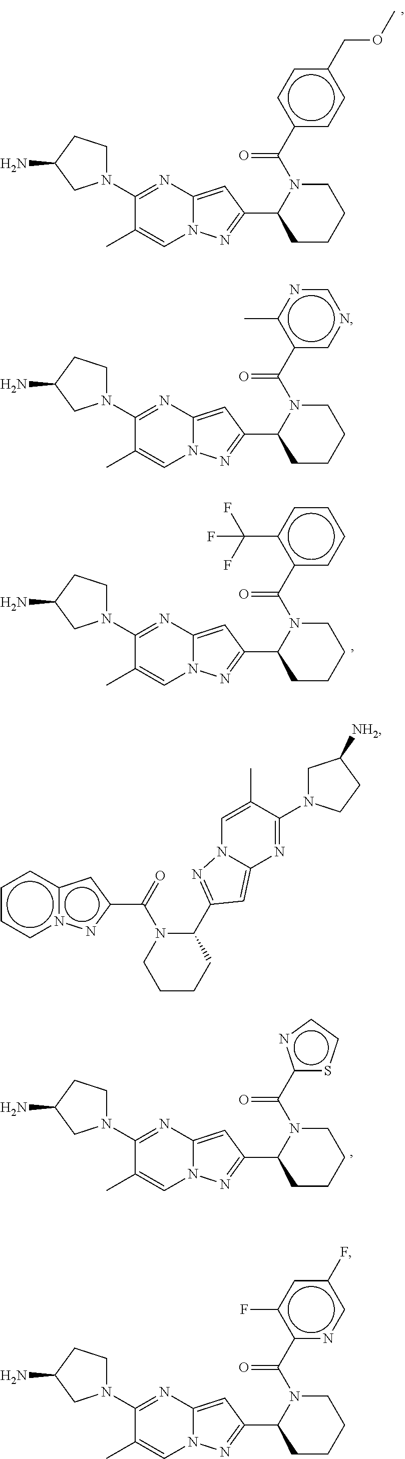

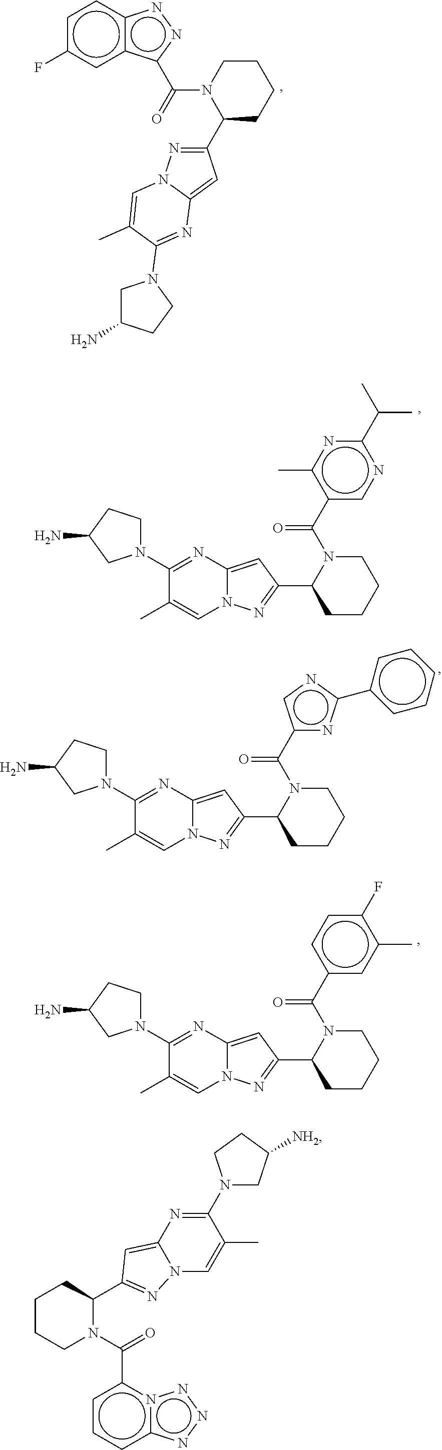

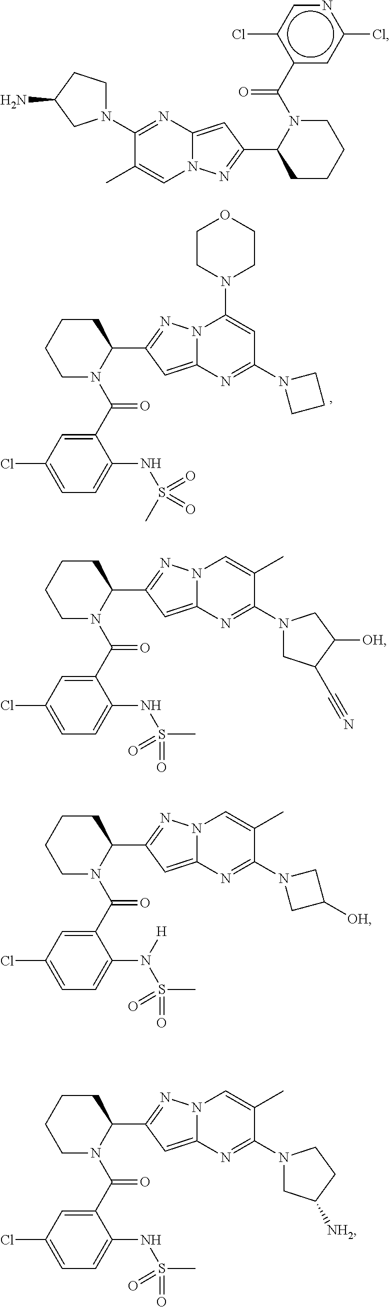

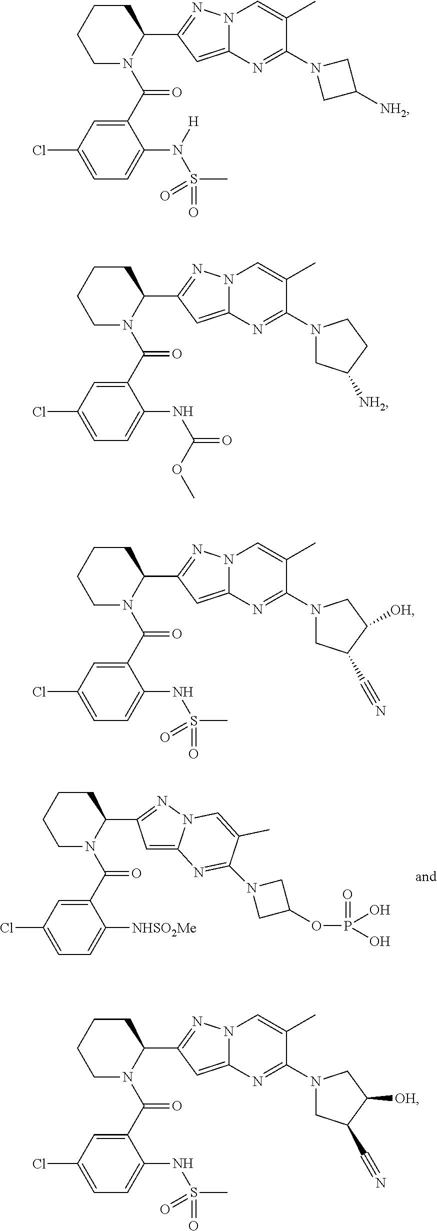

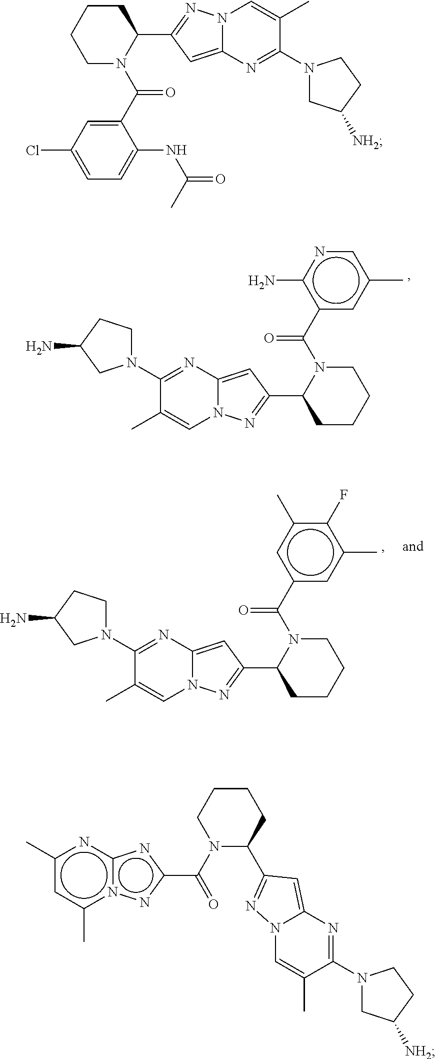

### Compounds of Formula (B21)

Compounds of the general Formula (B21) are described in PCT Publication No. WO 2014/031784, published Feb. 27, 2014, which is hereby incorporated by reference in its entirety. Formula (B21) has the structure:

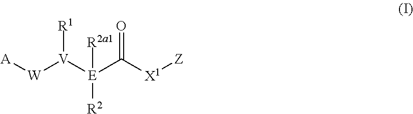

or a pharmaceutically acceptable salt thereof, wherein: A can be selected from an optionally substituted cycloalkyl, an optionally substituted cycloalkenyl, an optionally substituted aryl, an optionally substituted aryl(C₁₋₂ alkyl), an optionally substituted heteroaryl and an optionally substituted heterocyclyl; W can be O, S, C═O, C═S, NR^{3a3}, S═O, S(═O)₂ or —C(R^{1a1})(R^{1a2})—; V can be N or CH; E can be C or N; provided that when E is N, then R^{3a1} is absent; Z can be selected from

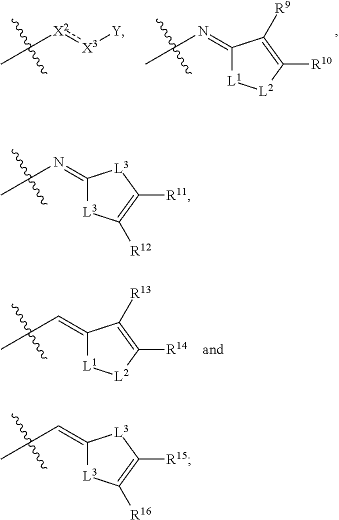

Y can be selected from an optionally substituted acylalkyl, an optionally substituted cycloalkyl, an optionally substituted cycloalkenyl, an optionally substituted aryl, an optionally substituted heteroaryl and an optionally substituted heterocyclyl; [symbol] between X² and X³ can represent a single or double bond between X² and X³; wherein when [symbol] is a double bond, then X¹ can be NR^{3a1} or CR^{3a2}R⁶; X² is N (nitrogen) or CR^{7a1}, and X³ can be N (nitrogen) or CR⁴; and when [symbol] is a single bond, then X¹ can be NR^{3a1} or CR^{3a2}R⁶, X² can be O, NR⁷, C(═O) or C(R^{7a2})(R^{7a3}), and X³ can be NR⁴, C(═O), CR⁴R⁸ or CH₂CH₂C(═O); or X¹, X² and X³ can be each independently C (carbon), N (nitrogen), O (oxygen) or C(═O), and form a mono-cyclic ring selected from an optionally substituted mono-cyclic heteroaryl and an optionally substituted mono-cyclic heterocyclyl by joining X¹ and X³ together; and provided that at least one of X¹, X² and X³ comprises a nitrogen atom, with the proviso that the valencies of X¹, X² and X³ are satisfied with a substituent selected from hydrogen and an optionally substituted C₁₋₄ alkyl; and X¹, X² and X³ are uncharged; L¹ can be —C(R¹⁷)₂—, —C(R¹⁸)₂C(R^{18a1})₂—, —C(R^{18a2})═C(R^{18a3})— or —C(R¹⁹)₂N(R^{19a1})—; L² can be —C(R²⁰)₂—, —N(R²¹)—, S, or O; each L³ can be independently —C(R²²)₂—, —C(R²³)₂C(R^{23a1})₂— or —C(R^{23a2})═C(R^{23a3})—; provided that when L is —C(R¹⁹)₂N(R^{19a1})—, then L² is —C(R²⁰)₂—; R¹ can be hydrogen or an unsubstituted C₁₋₄ alkyl; R^{1a1} and R^{1a2} can be each independently hydrogen, hydroxy or an unsubstituted C₁₋₄ alkyl; R² and R^{2a1} can be each independently selected from hydrogen, an optionally substituted C₁₋₄ alkyl, alkoxyalkyl, aminoalkyl, hydroxyalkyl, hydroxy, an optionally substituted aryl(C₁₋₆ alkyl), an optionally substituted heteroaryl(C₁₋₆ alkyl) and an optionally substituted heterocyclyl(C₁₋₆ alkyl); or R¹ and R², together with the atoms to which they are attached, can be joined to form an optionally substituted 5-membered heterocyclic ring or an optionally substituted 6-membered heterocyclic ring, R^{2a1} can be selected from hydrogen, an optionally substituted C₁₋₄ alkyl, alkoxyalkyl, aminoalkyl, hydroxyalkyl, hydroxy, an optionally substituted aryl(C₁₋₆ alkyl), an optionally substituted heteroaryl(C₁₋₆ alkyl) and an optionally substituted heterocyclyl(C₁₋₆ alkyl); R^{3a1}, R^{3a2} and R^{3a3} can be each independently hydrogen or an unsubstituted C₁₋₄ alkyl; R⁴ can be selected from hydrogen, an optionally substituted C₁₋₈ alkyl, an optionally substituted C₂₋₈ alkenyl, an optionally substituted C₂₋₈ alkynyl, an optionally substituted C₃₋₆ cycloalkyl, an optionally substituted aryl, an optionally substituted heteroaryl, an optionally substituted heterocyclyl, an optionally substituted C₃₋₆ cycloalkyl(C₁₋₆ alkyl), an optionally substituted aryl(C₁₋₆ alkyl), an optionally substituted heteroaryl(C₁₋₆ alkyl), an optionally substituted heterocyclyl(C₁₋₆ alkyl), halo(C₁₋₈ alkyl), an optionally substituted hydroxyalkyl, an optionally substituted alkoxyalkyl and cyano; R⁶, R⁷, and R^{7a1} can be each independently hydrogen or an unsubstituted C₁₋₄ alkyl; R^{7a2} and R^{7a3} can be each independently hydrogen or an unsubstituted C₁₋₄ alkyl; R⁸ can be hydrogen or optionally substituted C₁₋₄ alkyl; R⁹, R¹⁰, R¹¹, R¹², R¹³, R¹⁴, R¹⁵ and R¹⁶ can be each independently hydrogen or an unsubstituted C₁₋₄ alkyl; or R⁹ and R¹⁰, R¹¹ and R¹², R¹³ and R¹⁴, and R¹⁵ and R¹⁶, are each independently taken together form an optionally substituted cycloalkyl, an optionally substituted aryl, an optionally substituted heteroaryl or an optionally substituted heterocyclyl; and each R¹⁷, each R¹⁸, each R^{18a1}, R^{18a2}, R^{18a3}, each R¹⁹, R^{19a1}, each R²⁰, R²¹, each R²², each R²³, each R^{23a1}, R^{23a2} and R^{23a3} can be each independently hydrogen or an unsubstituted C₁₋₄ alkyl;

In some embodiments, Formula (B21) includes the following: provided that when X¹ is NR^{3a1}, X²═X³ is N═CR⁴, Y is an optionally substituted indolyl, then R⁴ is selected from of hydrogen, cyano, an optionally substituted C₂₋₆ alkyl, an optionally substituted acylalkyl, an optionally substituted hydroxyalkyl, an optionally substituted alkoxy(alkyl), an optionally substituted C₂₋₆ alkenyl, an optionally substituted C₂₋₆ alkynyl, haloalkyl, an optionally substituted C₃₋₆ cycloalkyl, an optionally substituted C₃₋₆ cycloalkyl(C₁₋₆ alkyl), an optionally substituted aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl).

In some embodiments, Formula (B21) includes the following: provided that when X¹ is NR^{3a1}, X²═X³ is N═CR⁴, Y is

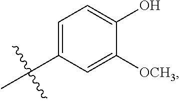 [SMILES: *c1ccc(O)c(OC)c1]

then R⁴ is selected from cyano, halo(C₁₋₈alkyl), an optionally substituted acylalkyl, an optionally substituted C₁₋₈ alkyl, an optionally substituted hydroxyalkyl, an optionally substituted alkoxy(alkyl), an optionally substituted C₂₋₈ alkenyl, an optionally substituted C₂₋₈ alkynyl, an optionally substituted C₃₋₆ cycloalkyl, an optionally substituted C₃₋₆ cycloalkyl(C₁₋₆ alkyl), an optionally substituted aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl).

In some embodiments, a compound of Formula (B21) can be selected from the following: 100, 101, 102, 103, 104, 105, 106, 107, 108, 109, 110, 111,112, 113, 114, 115, 116, 117, 118, 119, 120, 121,122, 123, 124, 125, 126, 127, 128, 129, 130, 131,132, 133, 134, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 242, 244, 245, 246A, 246B, 247, 300, 400, 401, 402, 403, 404, 405, 406, 407, 408, 409, 410, 411, 412, 413, 415, 416, 417, 419, 422, 423, 426, 427, 428, 429, 430, 431, 432, 433, 434, 435, 437, 438, 439, 440, 441, 442, 443, 444, 445, 448A, 448B, 449, 450, 453, 454, 455A, 455B, 456, 457, 458A, 458B, 459, 460, 461, 462A, 462B, 463A, 463B, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 400-1, 400-2, 400-3, 400-4, 400-5, 400-6, 400-7, 400-8, 400-9, 400-10, 400-11, 400-12, 400-13, 400-14, 400-15, 400-16, 400-17, 400-18, 400-19, 400-20, 400-21, 400-22, 400-24, 400-25, 400-26, 400-27, 400-28, 500, 501, 502, 503, 504, 505, 506, 507, 508, 509, 510, 511, 512, 513, 514A, 514B, 600, 601, 602, 603A, 603B, 604, 605, 606, 650, 651, 700, 701, 702, 703, 704, 705, 706, 707, 708, 709, 901,1206, 1352, 2300, 2301, 2302, 2303, 2304, 2400, 2401, 4105, 4304, 4305, 4306, 4307, 4308, 4309, 4310, 4311, 4312, 4313 and 4314.

In some embodiments, a compound of Formula (B21) can be selected from the following: 1200, 1202, 1204, 1209, 1211,1213, 1214, 1216, 1217, 1220, 1221,1223, 1224, 1225, 1226, 1227, 1230, 1231,1232, 1233, 1234, 1235, 1236, 1237, 1238, 1239, 1242, 1243, 1244, 1245, 1246, 1247, 1248, 1249, 1250, 1251,1252, 1253, 1255, 1256, 1257, 1258, 1300, 1301,1302, 1303, 1304, 1307, 1308, 1309, 1310, 1311,1312, 1313, 1314, 1315, 1316, 1317, 1318, 1319, 1320, 1321, 1322, 1323, 1325, 1326, 1327, 1328, 1329, 1330, 1331,1332, 1333, 1334, 1335, 1336, 1340, 1341, 1343, 1344, 1345, 1346, 1359, 1360, 1401,1402, 1403, 1404, 1405, 1501,1502, 1503, 1504, 1505, 1506, 1507, 1508, 1509, 1510, 1511,1512, 1513, 1514, 1515, 1516, 1517, 1518, 1519, 1520, 1521, 1522, 1523, 1524, 1525, 1526, 1527, 1528, 1529, 1530, 1531,1532, 1533, 1534, 1535, 1536, 1537, 1538, 1539, 1540, 1541,1601,1602, 1603, 1604, 1605, 1606, 1607, 1608, 1609, 1610, 1611,1612, 1613, 1614, 1615, 1616, 1617, 1618, 1619, 1620, 1621,1622, 1623, 1800, 1802, 1803, 1804, 1805, 1806, 1807, 1808, 1809, 1810, 1811,1812, 1813, 1814, 1815, 1816, 1817, 1818, 1819, 1820, 1821, 1822, 1823, 1824, 1825, 1826, 1829, 1830, 1831,1832, 1833, 1834, 1835, 1836, 1837, 1838, 1839, 1900, 1901,1902, 1903, 2000, 2100, 2101, 2103, 2104, 2105, 2106, 2107, 2108, 2109, 2111, 2112, 2113, 2114, 2115, 2504, 2506, 2507, 2508, 2601, 2602, 2603, 2604, 2605, 2613, 2615, 2617, 2618, 2619, 2620, 2621, 2622, 2624, 2626, 2627, 2638, 2641, 2642, 2643, 2644, 2645, 2646, 2647, 2648, 2649, 2650, 2651, 2652, 2654, 3302, 3800, 3903, 4002, 4201, 4202, 4203, 4204, 4205, 4206, 4207, 4208, 4209, 4210, 4212 and 4216.

In some embodiments, a compound of Formula (B21) can be selected from the following: 840, 1100, 1101,1201,1205, 1210, 1215, 1219, 1222, 1228, 1240, 1241, 2204, 2205, 2800, 2801, 3200, 3401, 3500, 3501, 3900 and 4303.

In some embodiments, a compound of Formula (B21) can be selected from the following: 900, 902, 903, 904, 908, 910, 917, 1000, 2803, 3300 and 4302.

In some embodiments, a compound of Formula (B21) can be selected from the following: 239, 240, 241, 2305, 2306 and 2802.

### Compounds of Formula (B22)

Compounds of the general Formula (B22) are described in PCT Publication No. WO 2015/026792, filed Aug. 19, 2014, which is hereby incorporated by reference in its entirety. Formula (B22) has the structure:

A-L-Y (I)

or a pharmaceutically acceptable salt thereof, wherein: L can be selected from:

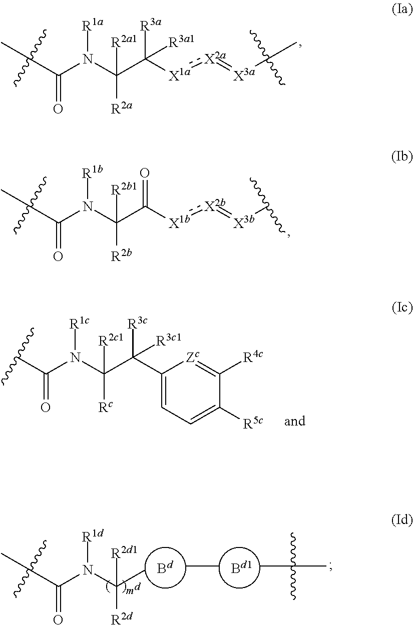

A can be selected from an optionally substituted cycloalkyl, an optionally substituted cycloalkenyl, an optionally substituted aryl, an optionally substituted aryl(C₁₋₂ alkyl), an optionally substituted heteroaryl and an optionally substituted heterocyclyl; Y can be selected from an optionally substituted cycloalkyl, an optionally substituted cycloalkenyl, an optionally substituted aryl, an optionally substituted heteroaryl and an optionally substituted heterocyclyl; R^{1a}, R^{1b}, R^{1c} and R^{1d} can be each independently hydrogen or an unsubstituted C₁₋₄ alkyl; R^{2a}, R^{2a1}, R^{2b}, R^{2b1}, R^{2c}R^{2c1}, R^{2d} and R^{2d1} can be each independently selected from can be hydrogen, an optionally substituted C₁₋₄ alkyl, an optionally substituted aryl(C₁₋₆ alkyl), an optionally substituted heterocyclyl(C₁₋₆ alkyl), an alkoxyalkyl, an aminoalkyl, a hydroxyalkyl and hydroxy; or R^{2a1} can be hydrogen, and R^{1a} and R^{2a} can be joined together with the atoms to which they are attached to form an optionally substituted 5 membered heterocyclyl or an optionally substituted 6 membered heterocyclyl, R^{2b1} can be hydrogen, and R^{1b} and R^{2b} can be joined together with the atoms to which they are attached to form an optionally substituted 5 membered heterocyclyl or an optionally substituted 6 membered heterocyclyl; [symbol] between X^{1a} and X^{2a} can represent a single or double bond between X^{1a} and X^{2a}; [symbol] between X^{2a} and X^{3a} can represent a single or double bond between X^{2a} and X^{3a}; provided that [symbol] between X^{1a} and X^{2a} and [symbol] between X^{2a} and X^{2a} cannot be both double bonds and at least one of [symbol] is a double bond; when [symbol] between X^{1a} and X^{2a} represents a double bond and [symbol] between X^{2a} and X^{3a} represents a single bond, then X^{1a} can be N or CR^{4a1}, X^{2a} can be N or CR^{5a} and X^{3a} can be NR^{6aa}, C(═O) or CR^{6a2}R^{6a3}; and when [symbol] between X^{1a} and X^{2a} represents a single bond and [symbol] between X^{2a} and X^{3a} represents a double bond, then X^{1a} can be NR^{4a} or CR^{4a2}R^{4a3}; X^{2a} can be N or CR^{5a} and X^{3a} can be N or CR^{6a}; or X^{1a}, X^{2a} and X^{3a} can be each independently C, N, O or C(═O), and form a ring or ring system selected from an optionally substituted aryl, an optionally substituted heteroaryl and an optionally substituted heterocyclyl by joining X^{1a} and X^{3a} together; with the proviso that the valencies of X^{1a}, X^{2a} and X^{3a} can be each independently satisfied with a substituent selected from hydrogen and an optionally substituted C₁₋₄ alkyl, and X^{1a}, X^{2a} and X^{3a} a are uncharged; R^{3a} and R^{3a1} can be each independently selected from hydrogen, hydroxy, halogen, amino, an optionally substituted C₁₋₄ alkyl, an optionally substituted C₂₋₄ alkenyl, an optionally substituted C₂₋₄ alkynyl, an optionally substituted C₃₋₆ cycloalkyl, an optionally substituted C₁₋₄ alkoxy, —O-carboxy, an optionally substituted heteroaryl, an optionally substituted heterocyclyl, CHF₂, CF₃ and

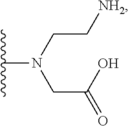 [SMILES: *N(CCN)CC(=O)O]

provided that R^{3a} and R^{3a1} cannot be both hydrogen; or R^{3a} and R^{3a1} can together form ═N—OR^{a}; or R^{3a} and R^{3a1} together with the atom to which they are attached can be joined to form an optionally substituted 3 membered ring, an optionally substituted 4 membered ring, an optionally substituted 5 membered ring or an optionally substituted 6 membered ring; R^{4a}, R^{4a1}; R^{4a2} and R^{4a3} can be each independently hydrogen or an unsubstituted C₁₋₄ alkyl; R^{5a} and R^{5a1} can be each independently be hydrogen or an unsubstituted C₁₋₄ alkyl; R^{6a} and R^{6a1} can be each independently hydrogen, an optionally substituted C₁₋₄ alkyl or an optionally substituted alkoxyalkyl; R^{6a2} and R^{6a3} can be each independently hydrogen or an unsubstituted C₁₋₄ alkyl; X^{1b}, X^{2b} and X^{3b} can be each independently C, N, O or C(═O), and form indicates a bi-cyclic ring selected from an optionally substituted bi-cyclic heteroaryl and an optionally substituted bi-cyclic heterocyclyl by joining X^{1b} and X^{3b} together, wherein [symbol] between X^{1b} and X^{2b} represents a single or double bond between X^{1b} and X^{2b}; [symbol] between X^{2b} and X^{3b} represents a single or double bond between X^{2b} and X^{3b}; and provided that at least one of X^{1b}, X^{2b} and X^{3b} A comprises a nitrogen atom and both [symbol] cannot be double bonds; with the proviso that the valencies of X^{1b}, X^{2b} and X^{3b} can be each independently satisfied with a substituent selected from hydrogen and an optionally substituted C₁₋₄ alkyl; and X^{1b}, X^{2b} and X^{3b} are uncharged; R^{3c} and R^{3c1} can be each independently selected from hydrogen, hydroxy, halogen, amino, an optionally substituted C₁₋₄ alkyl, an optionally substituted C₂₋₄ alkenyl, an optionally substituted C₂₋₄ alkynyl, an optionally substituted C₃₋₆ cycloalkyl, an optionally substituted C₁₋₄ alkoxy, —O-carboxy, an optionally substituted heteroaryl, an optionally substituted heterocyclyl, CHF₂, CF₃ and

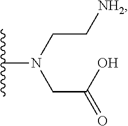 [SMILES: *N(CCN)CC(=O)O]

provided that R^{3c} and R^{3c1} cannot be both hydrogen; or R^{3c} and R^{3c1} together form ═N—OR^{c}; or R^{3c} and R^{3c1} together with the atom to which they are attached can be joined to form an optionally substituted 3 membered ring, an optionally substituted 4 membered ring, an optionally substituted 5 membered ring or an optionally substituted 6 membered ring; R^{a} and R^{c} can be each independently hydrogen or an unsubstituted C₁₋₄ alkyl; R^{4c} and R^{5c} can be taken together to form an unsubstituted aryl, an unsubstituted heteroaryl or an optionally substituted heterocyclyl; Z^{c} can be N or CH; m^{d} can be 0 or 1; ring B^{d} can be an optionally substituted C₅ cycloalkyl; ring B^{d1} can be an optionally substituted pyridinyl; and provided that when L is Formula (IIc), then Y is absent.

In some embodiments, Formula (B22) is not

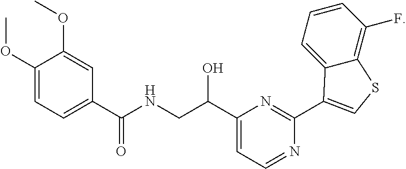 [SMILES: COc1ccc(C(=O)NCC(O)c2ccnc(-c3csc4c(F)cccc34)n2)cc1OC]

In some embodiments, a compound of Formula (B22) can be selected from the following: 1,13-1,100, 101,102, 103, 105, 106, 107, 108, 109, 110, 111,112, 113, 114, 115, 116, 116a, 116b, 117, 117a, 117b, 118, 118a, 118b, 119, 120, 120a, 120b, 121,122, 122a, 122b, 123, 124, 125, 126, 127, 128, 129, 131,132, 133, 134, 138, 139, 142, 143, 144, 145, 146, 147, 148, 151, 152, 153, 154, 155, 158, 159, 162, 163, 164, 165, 166, 167, 168, 169, 170, 171,172, 173, 174, 175, 176, 177, 178, 179, 180, 181,182, 183, 184, 185, 186, 187, 188, 189, 190, 191,192, 193, 194, 195, 196, 197, 198, 199, 200, 201, 202, 203, 204, 205, 206, 207, 208, 209, 210, 211, 212, 213, 214, 215, 216, 218, 219, 221, 223, 224, 225, 226, 227, 228, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 306, 307, 308, 309, 310, 312, 313, 314, 315, 316, 317, 318, 319, 320, 321, 322, 323, 324, 325, 326, 327, 328, 329, 330, 331, 332, 333, 334, 335, 336, 337, 338, 339, 340, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 475, 476, 477, 478, 479, 480, 481, 482, 483, 484, 485, 486, 487, 488, 489, 490, 491, 492, 493, 494, 495, 496, 497, 498a, 498b, 498c, 498d, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604a, 604b, 604c, 604d, 605a, 605b, 605c, 605d, 606, 607, 608, 609, 610, 611, 612, 613, 614, 615, 616, 617, 618, 619, 620, 621, 622, 623a, 623b, 624a, 624b, 625, 626, 627, 628, 629, 630, 631, 632, 633a, 633b, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 680, 681 and 682, or a pharmaceutically acceptable salt of the foregoing.

In some embodiments, a compound of Formula (B22) can be selected from the following: 629, 630, 631 and 632, or a pharmaceutically acceptable salt of the foregoing.

In some embodiments, a compound of Formula (B22) can be selected from the following: 149, 150, 156, 157, 160, 217, 220, 222, 229, 287, 302, 303, 304, 305, 311, 401, 473 and 474, or a pharmaceutically acceptable salt of the foregoing.

In some embodiments, a compound of Formula (B22) can be selected from the following: 130, 135, 140 and 141, or a pharmaceutically acceptable salt of the foregoing.

In some embodiments, a compound of Formula (B22) can be 104 or 161, or a pharmaceutically acceptable salt of the foregoing, as provided in (B22).

In some embodiments, a compound of Formula (B22) can be 136 or 137, or a pharmaceutically acceptable salt of the foregoing, as provided in (B22).

### Methods of Use

In some embodiments, a combination of compounds described herein (for example, a combination of one or more of compound (A) and one or more of compound (B), or a pharmaceutical acceptable salt of the foregoing) can be used to treat and/or ameliorate a paramyxovirus infection. In some embodiments, a combination of compounds described herein can be used to prevent a paramyxovirus infection. In some embodiments, a combination of compounds described herein can be used to inhibit the replication of a paramyxovirus. In some embodiments, a combination of compounds described herein can be used to inhibit the paramyxovirus polymerase complex.

In some embodiments, a combination of compounds described herein (for example, a combination of one or more of compound (A) and one or more of compound (B), or a pharmaceutical acceptable salt of the foregoing) can be used to treat and/or ameliorate a respiratory syncytial viral (RSV) infection. In some embodiments, a combination of compounds described herein can be used to prevent a respiratory syncytial viral infection. In some embodiments, a combination of compounds described herein can be used to inhibit the replication of a respiratory syncytial virus. In some embodiments, a combination of compounds described herein can be used to inhibit the RSV polymerase complex. In some embodiments, the RSV can be Type A. In other embodiments, the RSV can be Type B. In still other embodiments, the RSV can be Type A and B.

In some embodiments, a combination of compounds described herein (for example, a combination of one or more of compound (A) and one or more of compound (B), or a pharmaceutical acceptable salt of the foregoing) can be used to treat and/or ameliorate a HPIV-1 infection and/or HPIV-3 infection. In some embodiments, a combination of compounds described herein can be used to prevent a HPIV-1 infection and/or HPIV-3 infection. In some embodiments, a combination of compounds described herein can be used to inhibit the replication of HPIV-1 and/or HPIV-3. In some embodiments, a combination of compounds described herein can be used to inhibit the HPIV-1 polymerase complex and/or HPIV-3 polymerase complex.

In some embodiments, a combination of compounds described herein (for example, a combination of one or more of compound (A) and one or more of compound (B), or a pharmaceutical acceptable salt of the foregoing) can be used to treat and/or ameliorate a HPIV-2 infection and/or HPIV-4 infection. In some embodiments, a combination of compounds described herein can be used to prevent a HPIV-2 infection and/or HPIV-4 infection. In some embodiments, a combination of compounds described herein can be used to inhibit the replication of HPIV-2 and/or HPIV-4. In some embodiments, a combination of compounds described herein can be used to inhibit the HPIV-2 polymerase complex and/or HPIV-4 polymerase complex.

In some embodiments, a combination of compounds described herein (for example, a combination of one or more of compound (A) and one or more of compound (B), or a pharmaceutical acceptable salt of the foregoing) can be used to treat and/or ameliorate a metapneumoviral infection. In some embodiments, a combination of compounds described herein can be used to prevent a metapneumoviral infection. In some embodiments, a combination of compounds described herein can be used to inhibit the replication of a metapneumovirus. In some embodiments, a combination of compounds described herein can be used to inhibit the metapneumovirus polymerase complex. In some embodiments, including those of this paragraph, the metapneumovirus can be a human metapneumovirus.

In some embodiments, a combination of compounds described herein (for example, a combination of one or more of compound (A) and one or more of compound (B), or a pharmaceutical acceptable salt of the foregoing) can be used treat and/or ameliorate an upper respiratory viral infection caused by a paramyxovirus infection. In some embodiments, a combination of compounds described herein can be used treat and/or ameliorate a lower respiratory viral infection caused by a paramyxovirus infection. In some embodiments, a combination of compounds described herein can be used treat and/or ameliorate one or more symptoms of an infection caused by a paramyxovirus infection (such as those described herein). Respiratory infections include colds, croup, pneumonia, bronchitis and bronchiolitis. Symptoms can include a cough, runny nose, nasal congestion, sore throat, fever, difficulty breathing, abnormally rapid breathing, wheezing vomiting, diarrhea and ear infections. In some embodiments, a combination described herein can be used treat and/or ameliorate one or more symptoms of an infection caused by a virus selected from a RSV virus, a parainfluenza virus and a metapneumovirus (such as those described herein).

In some embodiments, a combination of compounds described herein (for example, a combination of one or more of compound (A) and one or more of compound (B), or a pharmaceutical acceptable salt of the foregoing) can be used treat and/or ameliorate bronchiolitis and/or tracheobronchitis due to a paramyxovirus infection. In some embodiments, a combination described herein can be used treat and/or ameliorate pneumonia due to a paramyxovirus infection. In some embodiments, a combination described herein can be used treat and/or ameliorate croup due to a paramyxovirus infection.

As used herein, the terms “prevent” and “preventing,” mean lowering the efficiency of viral replication and/or inhibiting viral replication to a greater degree in a subject who receives the compound compared to a subject who does not receive the compound. Examples of forms of prevention include prophylactic administration to a subject who has been or may be exposed to an infectious agent, such as a paramyxovirus (e.g., RSV).

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

Various indicators for determining the effectiveness of a method for treating a paramyxovirus viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator of disease response.

In some embodiments, a combination of compounds described herein (for example, a combination of one or more of compound (A) and one or more of compound (B), or a pharmaceutical acceptable salt of the foregoing) can reduce viral titers to undetectable levels, for example, less than 1.7 log₁₀ plaque forming units equivalents (PFUe)/mL, or less than 0.3 log₁₀ plaque forming units equivalents (PFUe)/mL. In some embodiments, a combination of compounds described herein can reduce the viral load compared to the viral load before administration of the combination (for example, 60 hours after receiving the initial dosage of the combination). In some embodiments, a combination of compounds described herein can reduce the viral load to lower than 1.7 log₁₀ (PFUe)/mL, or lower than 0.3 log₁₀ (PFUe)/mL. In some embodiments, a combination of compounds described herein can achieve a reduction in viral titer in the serum of the subject in the range of about 1.5-log to about a 2.5-log reduction, about a 3-log to about a 4-log reduction, or a greater than about 5-log reduction compared to the viral load before administration of the combination. For example, the viral load is measure before administration of the combination, and several hours after receiving the initial dosage of the combination (for example, 60 hours after receiving the initial dosage of the combination).

In some embodiments, a combination of compounds described herein (for example, a combination of one or more of compound (A) and one or more of compound (B), or a pharmaceutical acceptable salt of the foregoing) can result in at least a 1, 2, 3, 4, 5, 10, 15, 20, 25, 50, 75, 100-fold or more reduction in the replication of a paramyxovirus relative to pre-treatment levels in a subject, as determined several hours after receiving the initial dosage of the combination (for example, 60 hours after receiving the initial dosage of the combination). In some embodiments, a combination of compounds described herein can result in a reduction of the replication of a paramyxovirus relative to pre-treatment levels in the range of about 2 to about 5 fold, about 10 to about 20 fold, about 15 to about 40 fold, or about 50 to about 100 fold. In some embodiments, a combination of compounds described herein can result in a reduction of a paramyxovirus replication in the range of 1 to 1.5 log, 1.5 log to 2 log, 2 log to 2.5 log, 2.5 to 3 log, 3 log to 3.5 log or 3.5 to 4 log more reduction of a paramyxovirus replication compared to the reduction of a paramyxovirus reduction achieved by ribavirin (Virazole®), or may achieve the same reduction as that of ribavirin (Virazole®) therapy in a shorter period of time, for example, in one day, two days, three days, four days, or five days, as compared to the reduction achieved after 5 days of ribavirin (Virazole®) therapy.

After a period of time, infectious agents can develop resistance to one or more therapeutic agents. The term “resistance” as used herein refers to a viral strain displaying a delayed, lessened and/or null response to a therapeutic agent(s). For example, after treatment with an antiviral agent, the viral load of a subject infected with a resistant virus may be reduced to a lesser degree compared to the amount in viral load reduction exhibited by a subject infected with a non-resistant strain. In some embodiments, a combination of compounds described herein (for example, a combination of one or more of compound (A) and one or more of compound (B), or a pharmaceutical acceptable salt of the foregoing) can be administered to a subject infected with RSV that is resistant to one or more different anti-RSV agents (for example, ribavirin). In some embodiments, development of resistant RSV strains can be delayed when subjects are treated with combination of compounds described herein compared to the development of RSV strains resistant to other anti-RSV drugs administered as monotherapy.

In some embodiments, a combination of compounds described herein (for example, a combination of one or more of compound (A) and one or more of compound (B), or a pharmaceutical acceptable salt of the foregoing) can decrease the percentage of subjects that experience complications from a RSV viral infection compared to the percentage of subjects that experience complication being treated with ribavirin. For example, the percentage of subjects being treated with a combination of compounds described herein that experience complications can be 10%, 25%, 40%, 50%, 60%, 70%, 80% and 90% less compared to subjects being treated with ribavirin.

In some embodiments, a combination of compounds can include one or more of compound (A), or a pharmaceutically acceptable salt thereof. In some embodiments, a combination of compounds can include one or more of compound (B), or a pharmaceutically acceptable salt thereof. In some embodiments, one or more of compound (A), or a pharmaceutically acceptable salt thereof, can be administered with one or more of compound (B), or a pharmaceutically acceptable salt thereof, in a single pharmaceutical composition. In some embodiments, one or more of compound (A), or a pharmaceutically acceptable salt thereof, can be administered with one or more of compound (B), or a pharmaceutically acceptable salt thereof, as two or more separate pharmaceutical compositions. For example, compound (A), or a pharmaceutically acceptable salt thereof, can be administered in one pharmaceutical composition, and compound (B), or a pharmaceutically acceptable salt thereof, can be administered in a second pharmaceutical composition. In some embodiments, one or more of compound (A), or a pharmaceutically acceptable salt thereof, can be administered with at least one of compound (B), or a pharmaceutically acceptable salt thereof.

The order of administration of compound (A), or a pharmaceutically acceptable salt thereof, with compound (B), or a pharmaceutically acceptable salt thereof, can vary. In some embodiments, one or more of compound (A), or a pharmaceutically acceptable salt thereof, can be administered prior all of compound (B), or a pharmaceutically acceptable salt thereof. In other embodiments, one or more of compound (A), or a pharmaceutically acceptable salt thereof, can be administered prior to at least one compound (B), or a pharmaceutically acceptable salt thereof. In still other embodiments, one or more of compound (A), or a pharmaceutically acceptable salt thereof, can be administered concomitantly with one or more of compound (B), or a pharmaceutically acceptable salt thereof. In yet still other embodiments, one or more of compound (A), or a pharmaceutically acceptable salt thereof, can be administered subsequent to the administration of at least one compound (B), or a pharmaceutically acceptable salt thereof. In some embodiments, one or more of compound (A), or a pharmaceutically acceptable salt thereof, can be administered subsequent to the administration of all of compound (B), or a pharmaceutically acceptable salt thereof.

A potential advantage of utilizing a combination of compounds described herein (for example, a combination of one or more of compound (A) and one or more of compound (B), or a pharmaceutical acceptable salt of the foregoing) may be a reduction in the required amount(s) of one or more of compound (A), or a pharmaceutically acceptable salt thereof, and/or one or more of compound (B), or a pharmaceutically acceptable salt thereof, that is effective in treating a disease condition disclosed herein (for example, RSV), as compared to the amount required to achieve same therapeutic result when one or more of compound (B), or a pharmaceutically acceptable salt thereof, and/or one or more of compound (A), or a pharmaceutically acceptable salt thereof. For example, the amount of a one or more of compound (A), or a pharmaceutically acceptable salt thereof, and/or one or more of compound (B), or a pharmaceutically acceptable salt thereof, can be less compared to the amount of the aforementioned compounds needed to achieve the same viral load reduction when administered as a monotherapy. Another potential advantage of utilizing a combination described herein is that the use of two or more compounds having different mechanism of actions can create a higher barrier to the development of resistant viral strains compared to the barrier when a compound is administered as monotherapy. Additional advantages of utilizing a combination described herein may include little to no cross resistance between the compounds of the combination; different routes for elimination of the compounds of the combination; little to no overlapping toxicities between the compounds of the combination; little to no significant effects on cytochrome P450; and/or little to no pharmacokinetic interactions between the compounds of the combination.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Pharmaceutical Compositions

Some embodiments described herein relates to one or more pharmaceutical compositions, that can include one or more of compound (A), or a pharmaceutically acceptable salt thereof and/or one or more of compound (B), or a pharmaceutically acceptable salt thereof, and a pharmaceutically acceptable carrier, diluent, excipient or combination thereof.

The term “pharmaceutical composition” refers to a mixture of one or more of compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### EXAMPLES

Additional embodiments are disclosed in further detail in the following examples, which are not in any way intended to limit the scope of the claims.

### Example 1

### Preparation of Compounds 1-17

| Compound No. | Structure | Analytical Data |
|---|---|---|
| 1 | 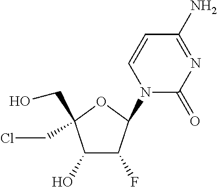 [SMILES: Nc1ccn([C@@H]2O[C@@](CO)(CCl)[C@@H](O)[C@H]2F)c(=O)n1] | 294 [M + H]⁺, 587 [2M + H]⁺ |
| 2 | 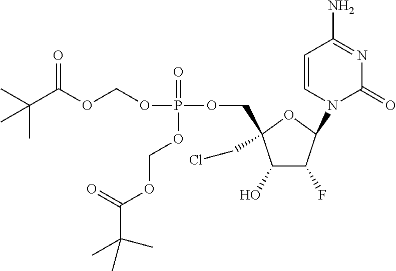 [SMILES: CC(C)(C)C(=O)OCOP(=O)(OCOC(=O)C(C)(C)C)OC[C@@]1(CCl)O[C@@H](n2ccc(N)nc2=O)[C@H](F)[C@@H]1O] | 646 [M + 46 − 1]. |
| 3 | 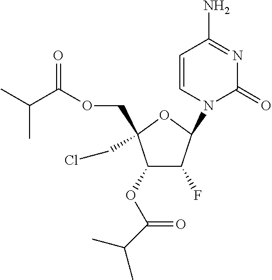 [SMILES: CC(C)C(=O)OC[C@@]1(CCl)O[C@@H](n2ccc(N)nc2=O)[C@H](F)[C@@H]1OC(=O)C(C)C] | 434 [M + 1] |
| 4 | 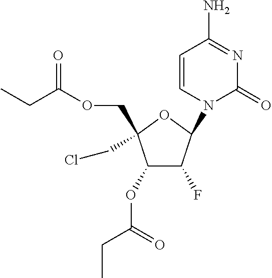 [SMILES: CCC(=O)OC[C@@]1(CCl)O[C@@H](n2ccc(N)nc2=O)[C@H](F)[C@@H]1OC(=O)CC] | 404 [M − 1] |
| 5 | 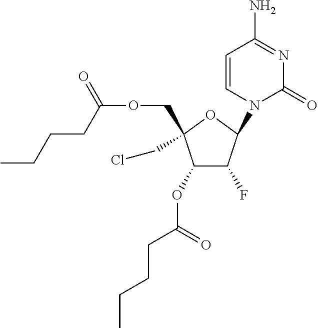 [SMILES: CCCCC(=O)OC[C@@]1(CCl)O[C@@H](n2ccc(N)nc2=O)[C@H](F)[C@@H]1OC(=O)CCCC] | 462 [M + 1] |
| 6 | 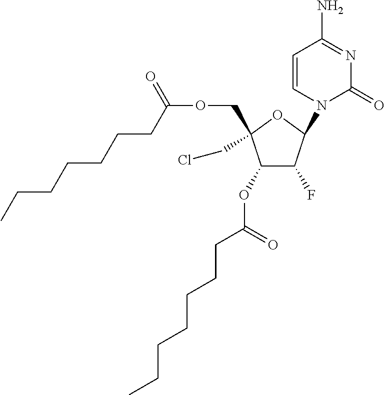 [SMILES: CCCCCCCC(=O)OC[C@@]1(CCl)O[C@@H](n2ccc(N)nc2=O)[C@H](F)[C@@H]1OC(=O)CCCCCCC] | 544 [M − 1] |
| 7 | 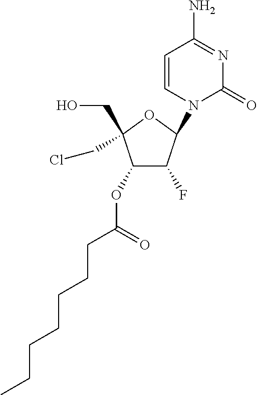 [SMILES: CCCCCCCC(=O)O[C@H]1[C@@H](F)[C@H](n2ccc(N)nc2=O)O[C@@]1(CO)CCl] | 418 [M − 1] |
| 8 | 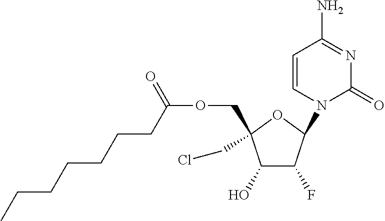 [SMILES: CCCCCCCC(=O)OC[C@@]1(CCl)O[C@@H](n2ccc(N)nc2=O)[C@H](F)[C@@H]1O] | 418 [M − 1] |
| 9 | 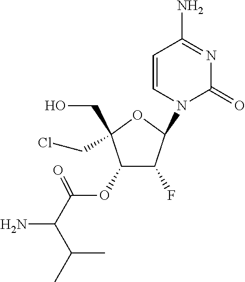 [SMILES: CC(C)C(N)C(=O)O[C@H]1[C@@H](F)[C@H](n2ccc(N)nc2=O)O[C@@]1(CO)CCl] | 391 [M − 1] |
| 10 | 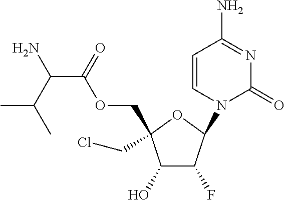 [SMILES: CC(C)C(N)C(=O)OC[C@@]1(CCl)O[C@@H](n2ccc(N)nc2=O)[C@H](F)[C@@H]1O] | 391 [M − 1] |
| 11 | 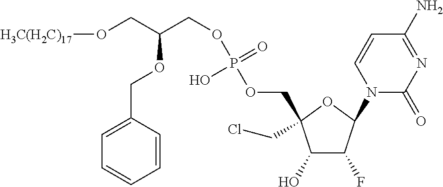 | 788.3 [M − H]⁺ |
| 12 | 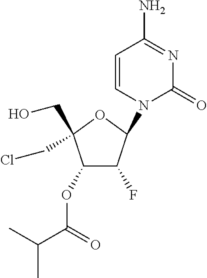 [SMILES: CC(C)C(=O)O[C@H]1[C@@H](F)[C@H](n2ccc(N)nc2=O)O[C@@]1(CO)CCl] | 362.1 [M + 1] |
| 13 | 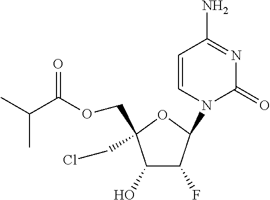 [SMILES: CC(C)C(=O)OC[C@@]1(CCl)O[C@@H](n2ccc(N)nc2=O)[C@H](F)[C@@H]1O] | 364 [M + 1] |
| 14 | 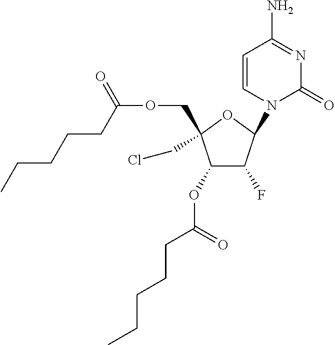 [SMILES: CCCCCC(=O)OC[C@@]1(CCl)O[C@@H](n2ccc(N)nc2=O)[C@H](F)[C@@H]1OC(=O)CCCCC] | 488 [M − 1] |
| 15 | 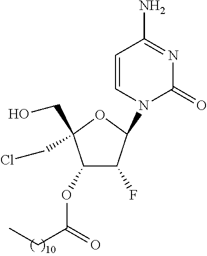 [SMILES: CCC(=O)O[C@H]1[C@@H](F)[C@H](n2ccc(N)nc2=O)O[C@@]1(CO)CCl] | 474 [M − 1] |
| 16 | 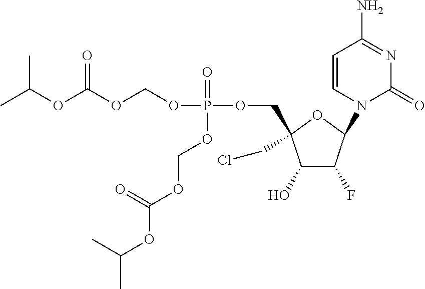 [SMILES: CC(C)OC(=O)OCOP(=O)(OCOC(=O)OC(C)C)OC[C@@]1(CCl)O[C@@H](n2ccc(N)nc2=O)[C@H](F)[C@@H]1O] | 606 [M + 1] |
| 17 | 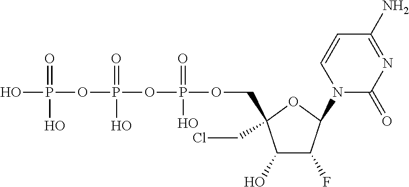 [SMILES: Nc1ccn([C@@H]2O[C@](CCl)(COP(=O)(O)OP(=O)(O)OP(=O)(O)O)[C@@H](O)[C@H]2F)c(=O)n1] | 532.1 [M − 1] |

Compounds 1-17 were prepared as described in U.S. Publication No. 2013/0165400, filed Dec. 20, 2012, CT Publication WO 2013/096679, filed Dec. 20, 2012 and Publication No. WO 2013/142525, Mar. 19, 2013, which are hereby incorporated by reference in their entireties.

### Example 2

### Preparation of Compound 18

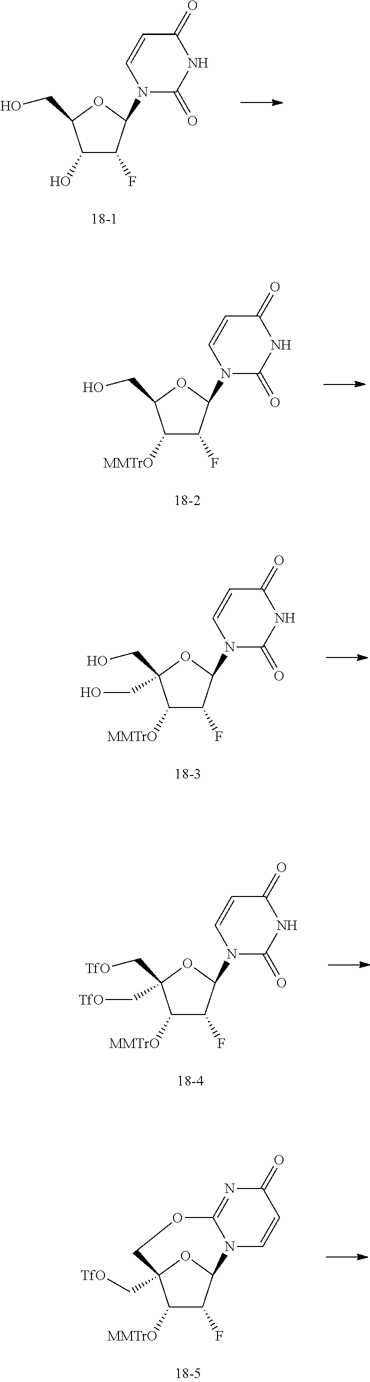

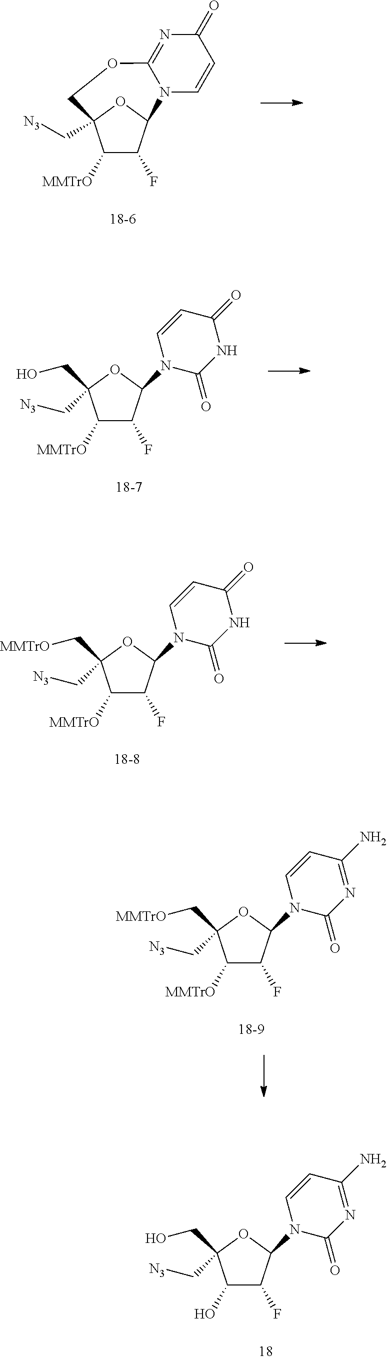

Preparation of (18-2):

To a solution of 18-1 (50 g, 203 mmol) in anhydrous pyridine (200 mL) was added TBDPS-Cl (83.7 g, 304 mmol). The reaction was allowed to proceed overnight at R.T. The solution was concentrated under low pressure to give a residue, which was partitioned between ethyl acetate and water. The organic layer was separated, washed with brine, dried over magnesium sulfate and concentrated under reduced pressure to give 5′-OTBDPS ether as a white foam (94 g).

To a solution of the 5′-OTBDPS ether (94.0 g, 194.2 mmol) in anhydrous DCM (300 mL) were added silver nitrate (66.03 g, 388.4 mmol) and collidine (235 mL, 1.94 mol). The mixture was stirred at R.T. After 15 mins, the mixture was cooled to 0° C., and monomethoxytrityl chloride (239.3 g, 776.8 mmol) was added as a single portion. After being stirred overnight at R.T., the mixture was filtered through Celite and the filtrate was diluted with TBME. The solution was washed successively with 1M citric acid, diluted brine and 5% sodium bicarbonate. The organic solution was dried over sodium sulfate and concentrated under vacuum to give the fully protected intermediate as a yellow foam.

This fully protected intermediate was dissolved in toluene (100 mL) and the solution was concentrated under reduced pressure. The residue was dissolved in anhydrous THF (250 mL) and treated with TBAF (60 g, 233 mmol). The mixture was stirred for 2 h at R.T., and the solvent was removed under reduced pressure. The residue was taken into ethyl acetate and the solution was washed first with saturated sodium bicarbonate and then with brine. After being dried over magnesium sulfate, the solvent was removed in vacuum and the residue was purified by column chromatography (50% EA in PE) to give 18-2 (91 g, 86.4%) as a white foam.

Preparation of (18-3):

To a solution of 18-2 (13.5 g, 26 mmol) in DCM (100 mL) was added pyridine (6.17 mL, 78 mmol). The solution was cooled to 0° C., and Dess-Martin periodinane (33.8 g, 78 mmol) was added as a single portion. The reaction mixture was stirred for 4 h at R.T., and quenched by the addition of Na₂S₂O₃ solution (4%) and sodium bicarbonate aqueous solution (4%) (the solution was adjusted to pH 6, ˜150 mL). The mixture was stirred for 15 mins. The organic layer was separated, washed with diluted brine and concentrated under reduced pressure. The residue was dissolved in dioxane (100 mL) and the solution was treated with 37% aqueous formaldehyde (21.2 g, 10 eq.) and 2N aqueous sodium hydroxide (10 eq.). The reaction mixture was stirred at R.T., overnight. After stirring for 0.5 h at R.T., the excess of aqueous sodium hydroxide was removed with saturated NH₄Cl (˜150 mL). The mixture was concentrated under reduced pressure, and the residue was partitioned between ethyl acetate and 5% sodium bicarbonate. The organic phase was separated, washed with brine, dried over magnesium sulfate and concentrated. The residue was purified by column chromatography (2% MeOH in DCM) to give the diol 18-3 (9.2 g, 83.6%) as a white foam.

Preparation of (18-4):

Compound 18-3 (23 g, 42.0 mmol) was co-evaporated with toluene twice. The residue was dissolved in anhydrous DCM (250 mL) and pyridine (20 mL). The solution was cooled to 0° C., and triflic anhydride (24.9 g, 88.1 mmol) was added dropwise over 10 mins. At this temperature, the reaction was stirred for 40 mins. The reaction was monitored by TLC (PE:EA=2:1 and DCM:MeOH=15:1). After completion, the reaction mixture was quenched with water (50 mL) at 0° C. The mixture was stirred for 30 mins, and extracted with EA. The organic phase was dried over Na₂SO₄ and filtered through a silica gel pad. The filtrate was concentrated under reduced pressure, and the residue was purified by column chromatography (50% EA in PE) to give 18-4 (30.0 g, 88.3%) as a brown foam.

Preparation of (18-5):

To a stirred solution of 18-4 (4.4 g, 5.42 mmol) in anhydrous DMF (50 mL) was added NaH (260 mg, 6.5 mmol) at 0° C. under nitrogen atmosphere. The solution was stirred at R.T., for 1.5 h. The solution was used for the next step without any further workup.

Preparation of (18-6):

To the stirred solution was added NaN₃ (1.5 g, 21.68 mmol) at 0° C. under nitrogen atmosphere, and the resulting solution was stirred at R.T. for 1.5 h. The reaction was quenched with water, extracted with EA, washed with brine, and dried over MgSO₄. The concentrated organic phase was used for the next step without further purification.

Preparation of (18-7):

To a solution of 18-6 (3.0 g, 5.4 mmol) in anhydrous 1,4-dioxane (18 mL) was added NaOH (5.4 mL, 2M in water) at R.T. The reaction mixture was stirred at R.T. for 3 h. The reaction was diluted with EA, washed with brine, and dried over MgSO₄. The concentrated organic phase was purified on a silica gel column (30% EA in PE) to give 1-7 (2.9 g, 93%) as a white foam.

Preparation of (18-8):

To a stirred solution of 18-7 (1.1 g, 2.88 mmol) in anhydrous DCM (10 mL) was added MMTrCl (1.77 g, 5.76 mmol), AgNO₃ (1.47 g, 8.64 mmol) and collidine (1.05 g, 8.64 mmol) at 25° C. under a N₂ atmosphere. The reaction was refluxed for 12 h. MeOH (20 mL) was added and the solvent was removed to dryness. The residue was purified on a silica gel column (20% EA in PE) to give 18-8 (1.6 g, 85.1%) as a white foam.

Preparation of (18-9):

To a stirred solution of 18-8 (800 mg, 0.947 mmol) in anhydrous MeCN (10 mL) were added TPSCl (570 mg, 1.89 mmol), DMAP (230 mg, 1.89 mmol) and TEA (190 mg, 1.89 mmol) at R.T. The mixture was stirred for 12 h. NH₄OH (25 mL) was added and the mixture was stirred for 2 h. The solvent was removed, and the residue was purified on a silica gel column as a yellow foam. Further purification by prep-TLC gave 18-9 (700 mg, 87.1%) as a white solid.

Preparation of (18):

Compound 18-9 (300 mg, 0.355 mmol) was dissolved in 80% of HCOOH (5 mL) at R.T. The mixture was stirred for 3 h, and monitored by TLC. The solvent was then removed and the residue was treated with MeOH and toluene (3 times). NH₃/MeOH was added and the mixture was stirred at R.T., for 5 mins. The solvent was removed and the residue was purified by column chromatography to give 18 (124 mg, 82.6%) as a white solid. ESI-LCMS: m/z 301.0 [M+H]⁺, 601.0 [2M+H]⁺.

### Example 3

### Preparation of Compound 19

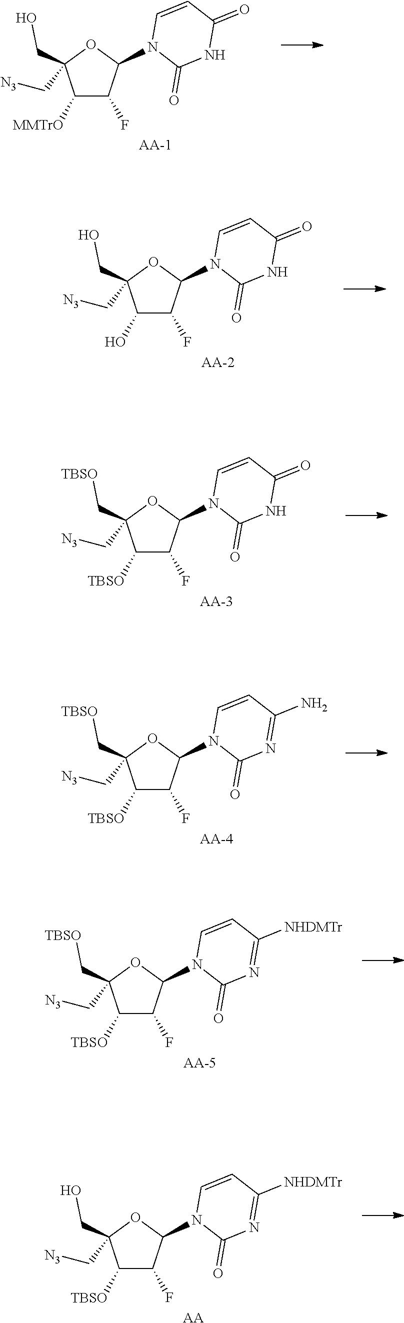

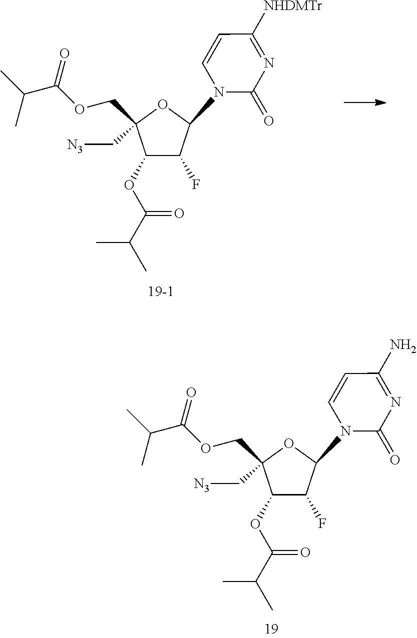

Preparation of (AA-2):

AA-1 (2.20 g, 3.84 mmol) was dissolved in 80% HCOOH (40 mL) at R.T. (18° C.). The mixture was stirred at R.T. for 12 h. The solvent was removed at low pressure. The residue was purified by column chromatography using 50% EA in Hexane to give AA-2 (1.05 g, 91.3%) as a white solid.

Preparation of (AA-3):

To a stirred solution of AA-2 (1 g, 3.32 mmol) in anhydrous pyridine (20 mL) was added TBSCl (747 mg, 4.98 mmol) and imidazole (451 mg, 6.64 mmol) at R.T. (16° C.) under N₂ atmosphere. The mixture was stirred at R.T. for 4 h. The resulting solution was concentrated to dryness under reduced pressure, and the residue was dissolved in EA (100 mL). The solution was washed with sat. NaHCO₃ solution and brine, and dried over anhydrous MgSO₄. The solution was concentrated to dryness, and the residue was purified on a silica gel column using 20% EA in Hexane to give AA-3 (1.4 g, 79.5%) as a white solid.

Preparation of (AA-4):

To a stirred solution of AA-3 (1.50 g, 2.83 mmol, 1.00 eq.) in anhydrous CH₃CN (28 mL) was added TPSCl (1.71 g, 5.80 mmol, 2.05 eq.), DMAP (691.70 mg, 5.66 mmol, 2.00 eq.) and TEA (573.00 mg, 5.66 mmol, 2.00 eq.) at R.T. (15° C.). The mixture was stirred for 2 h. NH₃.H₂O (20 mL) was added, and the mixture was stirred for 3 h. The mixture was extracted with EA (3×60 mL). The organic phase was washed with brine, dried over anhydrous Na₂SO₄ and concentrated at low pressure. The residue was purified on a silica gel column (30% EA in PE) to give AA-4 (2.3 g, crude) as a yellow foam.

Preparation of (AA-5):

To a stirred solution of AA-4 (1.90 g, 2.34 mmol) in anhydrous DCM (20 mL) was added DMTrCl (1.82 g, 3.49 mmol) and 2,4,6-trimethylpyridine (1.00 g, 8.25 mmol) at R.T. (15° C.) under N₂ atmosphere. The mixture was stirred at R.T. for 12 h. MeOH (20 mL) was added. The mixture was filtered, and the filtrate was concentrated to dryness. The residue was dissolved in EA (80 mL). The solution was washed with brine, dried over anhydrous Na₂SO₄ and concentrated at low pressure. The residue was purified on a silica gel column (5% MeOH in DCM) to give AA-5 (1.4 g, crude) as a white solid.

Preparation of (AA):

AA-5 (2.40 g, 2.60 mmol) was dissolved in TBAF (10 mL, 1M in THF). The mixture was stirred at R.T. (15° C.) for 30 mins. The mixture was concentrated to dryness, and the residue was dissolved in EA (60 mL). The solution was washed with brine, dried over MgSO₄ and concentrated under reduced pressure. The residue was purified on a silica gel column (5% MeOH in DCM) to give AA (1.50 g, 95.8%) as a white solid. ESI-MS: m/z 625.3 [M+Na]⁺.

Preparation of (19-1):

To a solution of AA (60.0 mg, 99.57 μmol, 1.00 eq.) in pyridine (1 mL) was added isobutyric anhydride (31.50 mg, 199.13 μmol, 2.00 eq.) in 1 portion at R.T. (15° C.) under N₂ atmosphere. The mixture was stirred at R.T. for 12 h. The mixture was concentrated, and the residue was partitioned between EA and water. The combined organic phases were washed with water and brine, and dried over anhydrous Na₂SO₄. The mixture was filtered, and the filtrate was concentrated to dryness. The residue was purified by silica gel chromatography (30% EA in PE) to afford 19-1 (59.00 mg, 79.77%) as a white solid.

Preparation of (19):

19-1 (57.00 mg, 76.74 μmol, 1.00 eq.) was dissolved in 80% CH₃COOH (8 mL). The solution was stirred at R.T. (15° C.) for 12 h. The mixture was concentrated to dryness. The residue was purified on a silica gel column (2.5% MeOH in DCM) to give 19 (23.00 mg, 68.05%) as a white foam. ESI-MS: m/z 441.2 [M+H]⁺, 463.2 [M+Na]⁺.

### Example 4

### Preparation of Compound 20

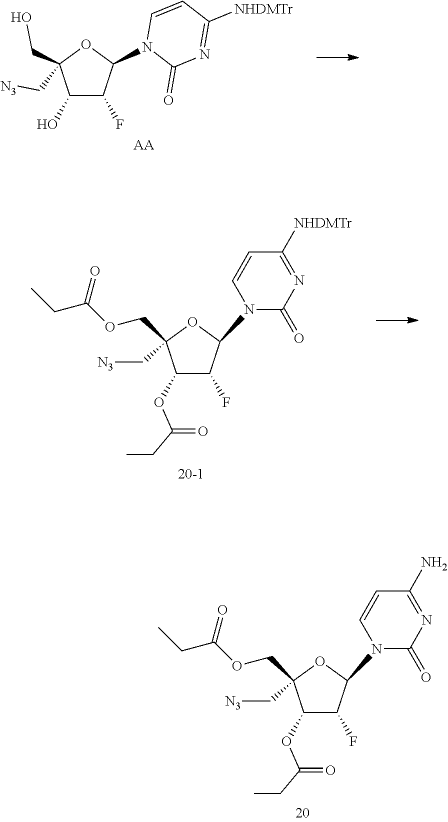

Preparation of (20-1):

20-1 was prepared in similar manner as 19-1 using AA (60.00 mg, 99.57 μmol, 1.00 eq.) in pyridine (1 mL) and propionic anhydride (25.92 mg, 199.13 μmol, 2.00 eq.). 20-1 (white solid, 56.00 mg, 78.69%).

Preparation of (20):

Compound 20 was prepared in similar manner as 19 using 20-1 (54.00 mg, 75.55 μmol, 1.00 eq.) 20 (white foam, 18.00 mg, 57.78%). ESI-MS: m/z 413.1 [M+H]⁺.

### Example 5

### Preparation of Compound 21

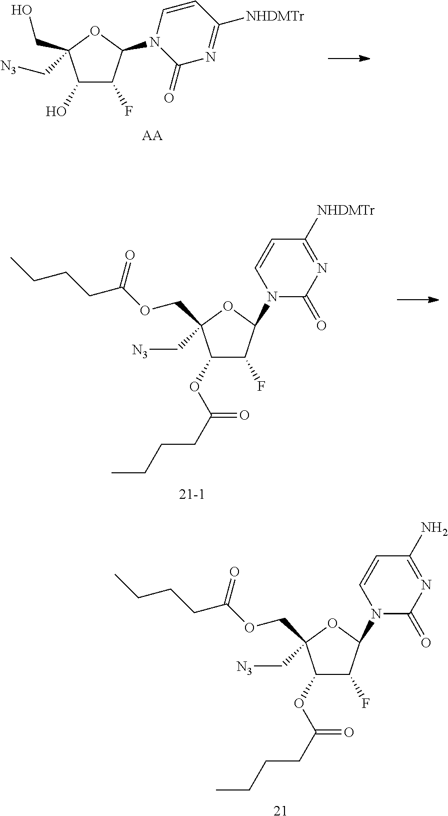

Preparation of (21-1):

21-1 was prepared in similar manner as 19-1 using AA (62.00 mg, 102.89 μmol, 1.00 eq.) in pyridine (1 mL) and pentanoic anhydride (38.32 mg, 205.77 μmol, 2.00 eq.). 21-1 (white solid, 60.00 mg, 75.65%).

Preparation of (21):

Compound 21 was prepared in similar manner as 19 using 21-1 (75.00 mg, 97.30 μmol, 1.00 eq.) 21 (white foam, 28.00 mg, 61.43%). ESI-MS: m/z 469.2 [M+H]⁺.

### Example 6

### Preparation of Compound 22

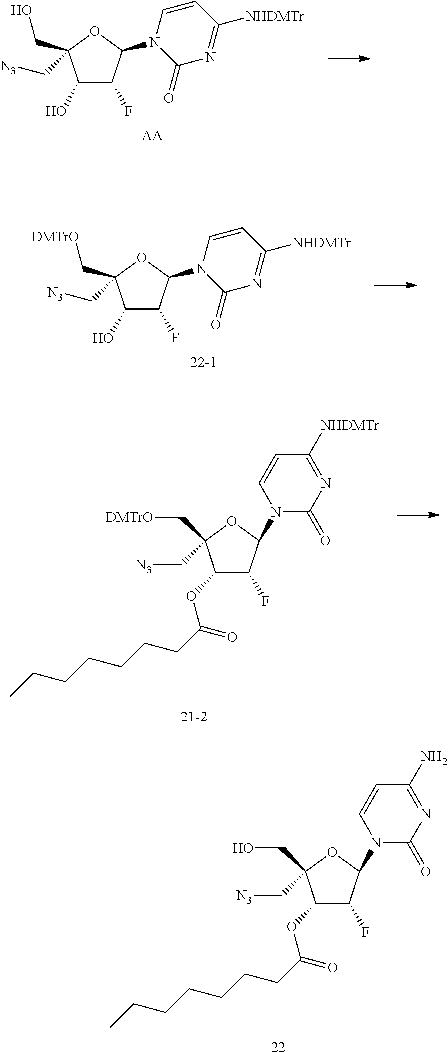

Preparation of (22-1):

To a stirred solution of AA-1 (300.0 mg, 497.83 μmol) in anhydrous pyridine (0.5 mL) was added DMTrCl (337.36 mg, 995.66 μmol) at R.T. (17° C.) under N₂ atmosphere. The solution was stirred at 50° C.˜60° C. for 12 h. The mixture was concentrated to dryness under reduced pressure, and the residue was dissolved in EA (40 mL). The solution was washed with brine, dried over anhydrous MgSO₄, and concentrated to dryness at low pressure. The residue was purified on a silica gel column using 20% EA in PE to give 22-1 (300 mg, 66.59%) as a white solid.

Preparation of (22-2):

To a stirred solution of 22-1 (100.00 mg, 110.50 μmol) in anhydrous pyridine (0.5 mL) was added DMAP (6.75 mg, 55.25 μmol), DCC (22.80 mg, 110.50 μmol) and n-actanoic acid (31.87 mg, 221.00 μmol) at R.T. (18° C.) under N₂ atmosphere. The solution was stirred at R.T. for 12 h. The solution was concentrated to dryness under reduced pressure. The residue was purified on a silica gel column using 15% EA in PE to give 22-2 (98.00 mg, 86.0%) as a white foam.

Preparation of (22):

22-2 (90.00 mg, 87.28 μmol) was dissolved in 80% CH₃COOH (20 mL) at R.T. (16° C.). The mixture was stirred R.T. for 12 h. The reaction was quenched with MeOH, and the mixture was concentrated to dryness. The residue was purified on a silica gel column (5% MeOH in DCM) to give 22 (33.00 mg, 88.7%) as a white solid. ESI-MS: m/z 427.2 [M+H]⁺.

### Example 7

### Preparation of Compound 23

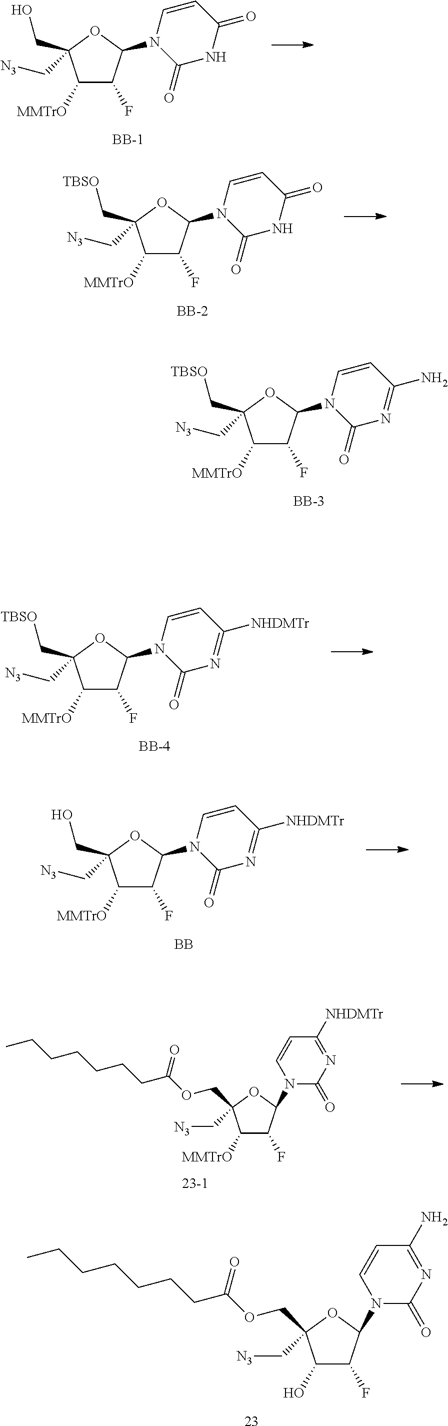

Preparation of (BB-2):

To a stirred solution of BB-1 (500.00 mg, 0.87 mmol) in anhydrous pyridine (1 mL) was added TBSCl (236.5 mg, 1.57 mmol) at 20° C. under N₂. The solution was stirred at 50° C.˜60° C. for 12 h. The solution was concentrated to dryness under reduced pressure. The residue was dissolved in EA (50 mL). The solution was washed with sat. NaHCO₃ solution and brine, and dried over anhydrous MgSO₄. The solution was filtered, and the filtrate was concentrated to dryness. The residue was purified on a silica gel column to give BB-2 (510.00 mg, 85.06%) as a white solid.

Preparation of (BB-3):

To a stirred solution of BB-2 (430.00 mg, 625.15 mmol) in anhydrous MeCN (6 mL) was added TPSCl (368.65 mg, 1.25 mmol), DMAP (152.75 mg, 1.25 mmol) and TEA (126.52 mg, 1.25 mmol) at R.T. The mixture was stirred for 2 h. NH₄OH (8 mL) was added, and the mixture stirred for 3 h. The mixture was extracted with EA (3×40 mL). The organic phase was washed with brine, dried over anhydrous Na₂SO₄ and concentrated at low pressure. The residue was purified on a silica gel column (25% EA in PE) to give BB-3 (500 mg of crude) as a yellow foam.

Preparation of (BB-4):

To a stirred solution of BB-3 (500 mg of crude, 0.72 mmol) in anhydrous DCM (7 mL) was added DMTrCl (365 mg, 1.0 mmol) and collidine (305 mg, 2.5 mmol) and AgNO₃ (184 mg, 1.08 mmol) at R.T. (15° C.) under N₂ atmosphere. The mixture was stirred at R.T. for 12 h. MeOH (5 mL) was added. The mixture was filtered, and the filtrate was concentrated to dryness. The residue was dissolved in EA (50 mL). The solution was washed with brine, dried over anhydrous Na₂SO₄ and concentrated at low pressure. The residue was purified on a silica gel column (5% MeOH in DCM) to give BB-4 (500 mg, 70.3%) as a white solid.

Preparation of (BB):

BB-4 (1.00 g, 1.01 mmol) was dissolved in TBAF (5 mL, 1M in THF) and stirred at R.T. for 30 mins. The mixture was diluted with EA (100 mL). The mixture was washed with water and brine, and dried over anhydrous MgSO₄. The organic phase was concentrated to dryness. The residue was purified on the silica gel column (30% EA in PE) to give BB (0.80 g, 91.5%) as a white solid. ESI-MS: m/z 873.7 [M+1]′.

Preparation of (23-1):

To a solution of BB (100.00 mg, 114.29 μmol) in anhydrous pyridine (1.5 mL) was added DMAP (2.79 mg, 22.86 μmol), DCC (70.75 mg, 342.88 μmol) and n-octanoic acid (49.45 mg, 342.88 μmol) at R.T. (18° C.) under N₂ atmosphere. The solution was stirred at R.T. for 12 h. The solution was concentrated to dryness under reduced pressure. The residue was purified on a silica gel column using 15% EA in PE to give 23-1 (95.00 mg, 83.03%) as a white foam.

Preparation of (23):

23-1 (110.00 mg, 109.87 μmol) was dissolved in 80% CH₃COOH (25 mL) at R.T. (15° C.). The mixture was stirred for 12 h. The reaction was quenched with MeOH, and the solution was concentrated to dryness. The residue was purified on a silica gel column (5% MeOH in DCM) to give 23 (30.00 mg, 64.03%) as a white solid. ESI-MS: m/z 427.2 [M+H]⁺.

### Example 8

### Preparation of Compound 24

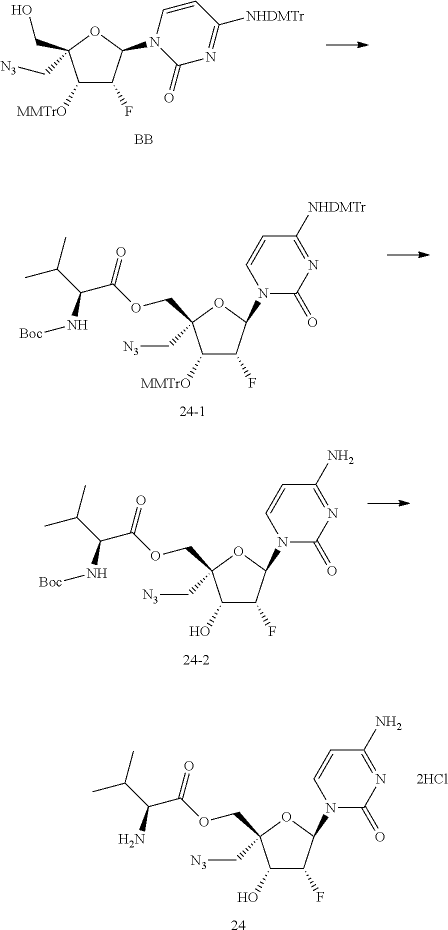

Preparation of (24-1):

To a solution of N-Boc-L-Valine (620.78 mg, 2.86 mmol) and TEA (144.57 mg, 1.43 mmol) in anhydrous THF (2.5 mL) was added BB (250.00 mg, 285.73 μmol). The mixture was co-evaporated with pyridine and toluene to remove water. The residue was dissolved in THF (2.5 mL). DIPEA (369.28 mg, 2.86 mmol) was added, followed by addition of BOP-Cl (363.68 mg, 1.43 mmol) and 3-nitro-1H-1,2,4-triazole (162.95 mg, 1.43 mmol) at R.T. (18° C.). The mixture was stirred at R.T. for 12 h and then diluted with EA (40 mL). The solution was washed with brine, dried over anhydrous Na₂SO₄ and concentrated to dryness at low pressure. The residue was purified on a silica gel column (30% EA in PE) to give 24-1 (220 mg, crude) as a white foam.

Preparation of (24-2):

24-1 (250.0 mg, 232.73 μmol) was dissolved in 80% CH₃COOH (30 mL). The solution was heated to 50° C. and stirred for 12 h. The reaction was quenched with MeOH, and the solution was concentrated to dryness. The residue was purified on a silica gel column (5% MeOH in DCM) to give 24-2 (80.00 mg, 68.82%) as a white foam.

Preparation of (24):

24-2 (78.00 mg, 156.16 μmol) was dissolved in HCl/dioxane (1.5 mL) and EA (1.5 mL) at R.T. (19° C.). The mixture was stirred at R.T. for 30 mins. The solution was concentrated to dryness at low pressure The residue was purified by prep-HPLC to give 24 (23 mg, 31.25%) as a white solid. ESI-MS: m/z 400.20 [M+H]⁺, 799.36 [2M+H]⁺.

### Example 9

### Preparation of Compound 25

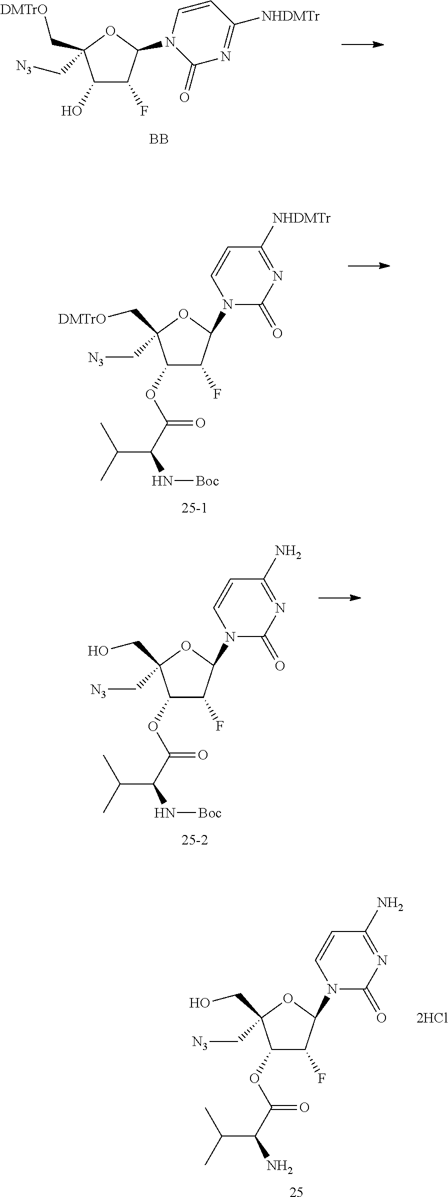

Preparation of (25-1):

25-1 was prepared in similar manner as 24-1 using BB (250.0 mg, 276.25 μmol), (2S)-2-(tert-butoxycarbonylamino)-3-methyl-butanoic acid (360.11 mg, 1.66 mmol) and TEA (83.86 mg, 828.75 μmol). 25-1 (white foam, 220.0 mg, 72.12%).

Preparation of (25-2):

25-2 was prepared in similar manner as 24-2 using 25-1 (230.00 mg, 208.29 μmol, 1.00 eq.). 25-2 (white foam, 80.00 mg, 77.66%).

Preparation of (25):

25 was prepared in similar manner as 24 using 25-2 (100.00 mg, 200.20 μmol, 1.00 eq.). 25 (white solid, 56 mg, 59.57%). ESI-MS: m/z 400.0 [M+H]⁺, 422.1 [M+Na]⁺; 799.1 [2M+H]⁺, 821.2 [2M+Na]⁺.

### Example 10

### Preparation of Compound 27

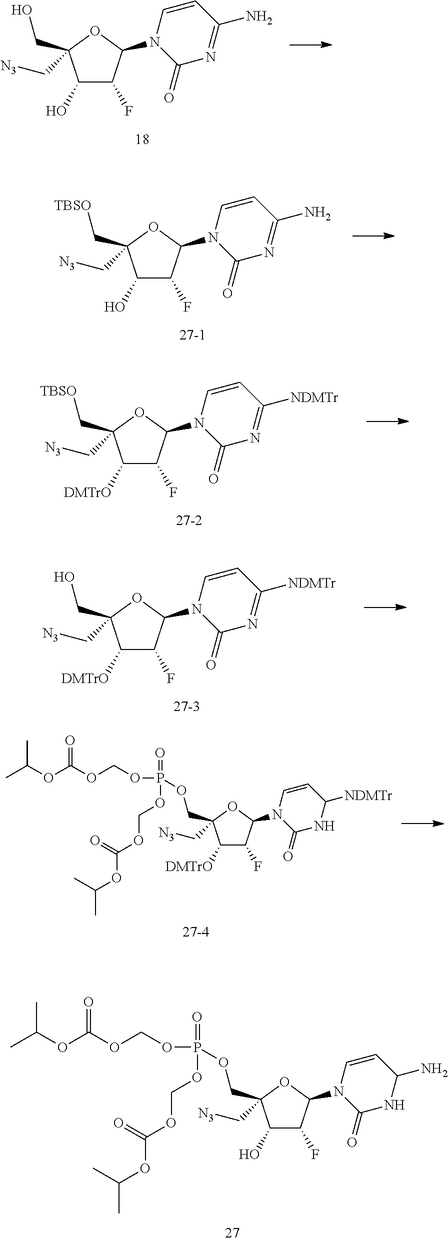

Preparation of (27-1):

To a solution of 18 (200 mg, 0.67 mmol) in anhydrous pyridine (5 mL) was added TBSCl (120 mg, 0.8 mmol) at R.T. The mixture was stirred overnight, and the reaction mixture was diluted with EA. The mixture was washed with NaHCO₃ aq. solution and brine. The organic layer was dried, filtered and concentrated to give residue, which was purified by silica gel column chromatography (5% MeOH in DCM to 25% MeOH in DCM to give 27-1 (153 mg, 55%) as a white solid.

Preparation of (27-2):

To a solution of 27-1 (54 mg, 0.13 mmol) in anhydrous DCM (2 mL) was added collidine (95 μL, 0.78 mmol), DMTrCl (262 mg, 0.78 mmol) and AgNO₃ (66 mg, 0.39 mmol) at R.T. The mixture was stirred overnight, and then diluted with DCM (5 mL). The mixture was filtered through a pre-packed celite funnel, and the filtrate was washed with NaHCO₃ aq. solution, 1.0 M citric acid solution and then brine. The organic layer was dried over Na₂SO₄, and concentrated at low pressure to give a residue. The residue was purified by silica gel column chromatography (25% EA in PE to 100% EA) to give 27-2 (83.5 mg, 63.6%).

Preparation of (27-3):

To a solution of 27-2 (83 mg, 0.081 mmol) in THF (1 mL), was added a 1M solution of TBAF in THF (0.122 mL, 0.122 mmol) at ice bath temperature. The mixture was stirred for 1.5 h. The mixture was diluted with EA, and washed with water and brine. The organic layer was dried and concentrated to give the crude product, which was purified by silica gel column chromatography (CM to 5% MeOH in DCM) to give 27-3 (66.6 mg, 91%) as a white foam.

Preparation of (27-4):

Compound 27-3 (66.6 mg, 0.074 mmol) was co-evaporated with toluene and THF (3×). Bis(POC)phosphate (33 mg, 0.96 mmol) was added, and then co-evaporated with toluene (3×). The mixture was dissolved in anhydrous THF (1.5 mL) and cooled in an ice bath (0 to 50 C). 3-nitro-1,2,4-triazole (13 mg, 0.11 mmol), diisopropylethyl amine (54 μL, 0.3 mmol), and BOP-Cl (28 mg, 0.11 mmol) were added successively. The mixture was stirred 2 h at 0 to 5° C., diluted with EtOAc, washed with 1.0M citric acid, sat. aq. NaHCO₃ and brine, and dried with Na₂SO₄. The residue was purified on silica (10 g column) with CH₂Cl₂:i-PrOH (4-10% gradient) to give 27-4 (68 mg, 76%) as a white solid.

Preparation of (27):

27-4 (68 mg, 0.07 mmol) was dissolved in 80% HCOOH. The mixture was stirred at R.T. for 2 h. The solvents were evaporated at R.T. and co-evaporated with toluene (3×). The residue was dissolved in 50% CH₃CN/H₂O, was purified on a reverse-phase HPLC (C18) using CH₃CN and H₂O. The product was lyophilization to give 27 (4.8 mg, 14%) as a white foam. ESI-LCMS: m/z=613.1 [M+H]⁺, 1225.2 [2M+H]⁺.

### Example 11

### Preparation of Compound 28

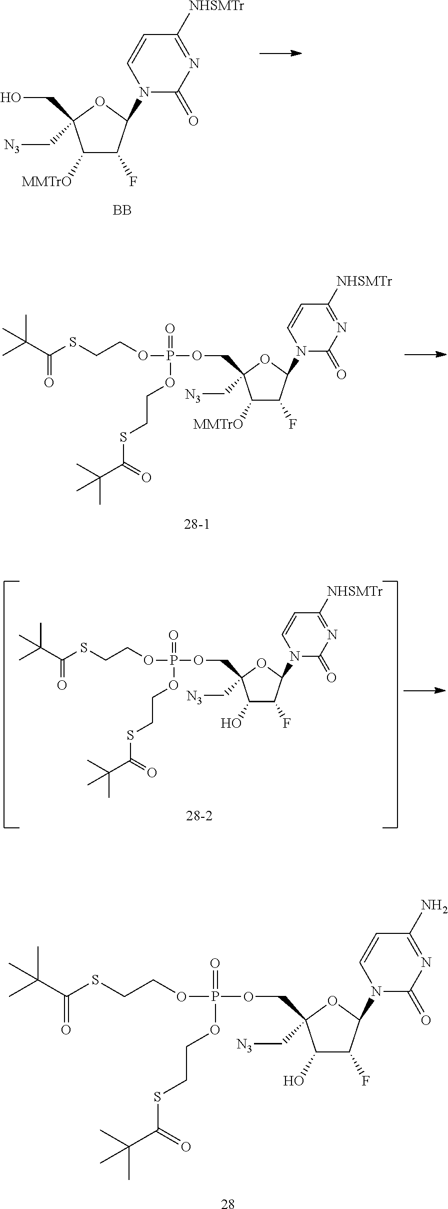

Preparation of (28-1):

To a solution of BB (100 mg, 0.114 mmol) in anhydrous CH₃CN (2 mL) were added a solution of bis-SATE-phosphoramidate (62.2 mg, 0.14 mmol) in CH₃CN (1 mL) followed by 5-ethylthio-1H-tetrazole in CH₃CN (0.25M; 0.56 mL, 0.14 mmol) at 0 to 5° C. dropwise. The mixture was stirred 2 h at 0 to 5° C. under Ar. A solution of 77% m-CPBA (49 mg, 0.22 mmol) in DCM (1 mL) was added, and the mixture was stirred 2 h at 0 to 5° C. under Ar. The mixture was diluted with EtOAc (50 mL), washed with 1.0M citric acid, sat. NaHCO₃, and brine, and dried with MgSO₄. The mixture was filtered and the solvents were evaporated in vacuo. The residue was purified on silica (10 g column) with EA/hexanes (10-100% gradient) to give 28-1 (72 mg, 50.8%) as a white solid.

Preparation of (28):

28-1 (72 mg, 0.056 mmol) was dissolved in anhydrous CH₃CN (1.0 mL), and 4N HCl in dioxane (87 μL, 0.35 mmol) was added at 0 to 5° C. The mixture was stirred at R.T. for 2 h. Intermediate 28-2 was observed by LCMS. The solvents were evaporated at R.T. and co-evaporated with toluene (3×). The residue obtained was re-dissolved in 80% HCOOH (2 mL). The mixture was stirred at R.T. for 4.5 h. The solvents were evaporated at R.T. and co-evaporated with toluene (3×). Anhydrous EtOH (3×5 mL) was added. The residue was dissolved in 50% CH₃CN/H₂O, purified on a reverse-phase HPLC (C18) using CH₃CN and H₂O, and lyophilized to give 28 (19.2 mg) as a white foam. ESI-LCMS: m/z=669.2 [M+H]⁺, 1337.25 [2M+H]⁺.

### Example 12

### Preparation of Compound 29

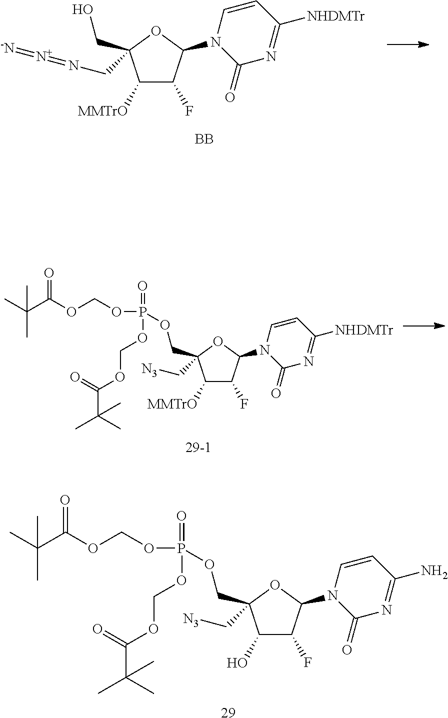

Preparation of (29-1):

29-1 (98 mg, 72.6%) was prepared in the same manner from BB (100 mg, 0.114 mmol) and bis(tert-butoxycarbonyloxymethyl)phosphate (83 mg, 0.35 mmol) with DIPEA (126 μL, 0.69 mmol), BOP-Cl (87 mg, 0.34 mmol), and 3-nitro-1,2,4-triazole (39 mg, 0.34 mmol) in THF (1.5 mL) in the same manner as 27-4.

Preparation of (29):

29-1 (98 mg, 0.083 mmol) was dissolved in anhydrous CH₃CN (0.5 mL), and 4N HCl in dioxane (34 μL, 0.135 mmol) was added at 0 to 5° C. The mixture was stirred at R.T. for 3 h. Anhydrous EtOH (200 μL) was added. The solvents were evaporated at R.T. and co-evaporated with toluene (3×). The residue was purified on silica (10 g column) with MeOH/CH₂Cl₂ (5-7% gradient) and lypholized give 29 (30.2 mg, 60%). ESI-LCMS: m/z=609.15 [M+H]⁺, 1217.3 [2M+H]⁺.

### Example 13

### Preparation of Compound 30

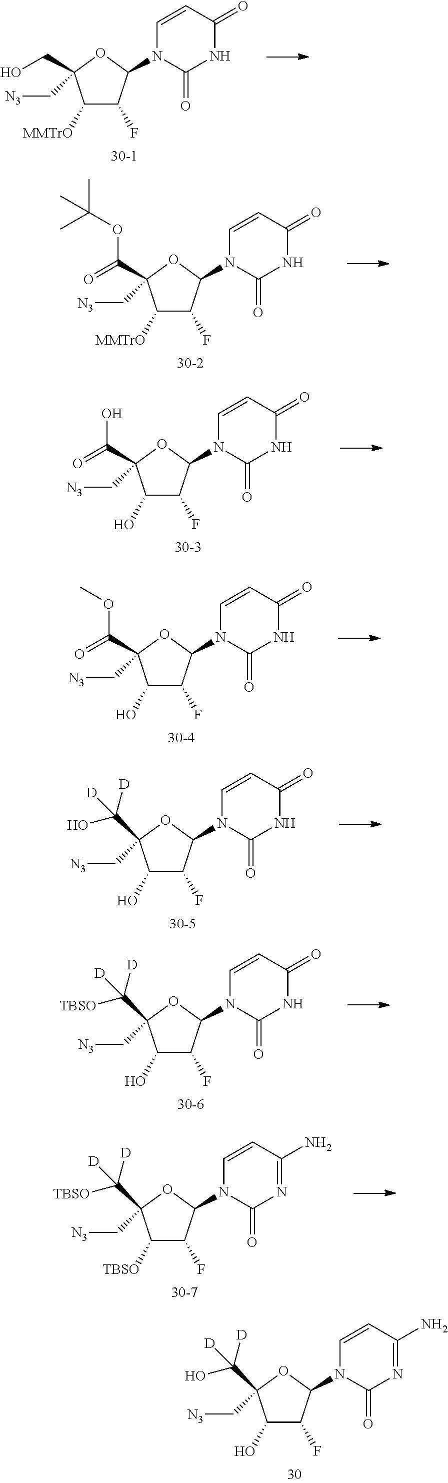

To a stirred solution of 30-1 (3.00 g, 5.23 mmol) in anhydrous DCM (36 mL) was added PDC (3.94 g, 10.46 mmol), Ac₂O (5.34 g, 52.30 mmol) and 2-methylpropan-2-ol (7.75 g, 104.60 mmol) at RT. The mixture was stirred at RT for 15 h. The mixture was loaded on a very short silica gel column and eluted with EA. The fractions containing the product were combined and concentrated under reduced pressure. The residue was purified by column chromatography (20% EA in PE) to give 30-2 (2.40 g, 71.3%) as a white foam.

To a stirred solution of 30-2 (2.00 g, 3.26 mmol) in DCM (30 mL) was added TFA (15 mL). The mixture was stirred at RT for 1.5 h. The mixture was concentrated under reduced pressure to give 30-3 (1.00 g, crude), which was used in the next step without further purification.

Crude 30-3 (1.00 g, crude) was dissolved in a mixture of toluene (25 mL) and MeOH (20 mL). TMS-diazomethane (2 M, 3.17 mL) was added. After stirring for 2 h, the mixture was concentrated under reduced pressure at RT. The residue was diluted with EA (25 mL), washed with water (25 mL), dried over anhydrous MgSO₄, filtered and concentrated under reduced pressure. The residue was purified by column chromatography (2% MeOH in DCM) to give 30-4 (451 mg, 43.2%) as a white solid. The aqueous phase was concentrated to give 30-3 (500 mg, 50.0%) as a white solid.

To a solution of 30-4 (451 mg, 1.37 mmol) in anhydrous CD₃OD (18 mL) was added NaBD₄ (344 mg, 8.22 mmol) at RT. The mixture was stirred at RT for 1 h. The reaction was quenched with CD₃OD (0.2 mL) and neutralized with AcOH (0.2 mL). The mixture was concentrated under reduced pressure. The residue was purified by column chromatography (4% MeOH in DCM) to give 30-5 (410 mg, 98.7%) as a white solid.

To a stirred solution of 30-5 (410 mg, 1.35 mmol) in pyridine (2.5 mL) was added imidazole (459 mg, 6.75 mmol) and TBSCl (610 mg, 4.05 mmol) at RT. The mixture was stirred at 60° C. for 10 h. The mixture was concentrated under reduced pressure. The residue was diluted with EA (20 mL) and washed with brine (20 mL). The organic layer was dried over MgSO₄ and filtered. The filtrate was concentrated under reduced pressure. The residue was purified by column chromatography (10% EA in PE) to give 30-6 (440 mg, 61.3%) as a white solid.

To a solution of 30-6 (440 mg, 827 μmol) in anhydrous MeCN (4 mL) were added DMAP (253 mg, 2.07 mmol), Et₃N (209.32 mg, 2.07 mmol) and 2,4,6-triisopropylbenzene-1-sulfonyl chloride (626.50 mg, 2.07 mmol) at RT. The mixture was stirred at RT for 16 h. NH₃.H₂O (2 mL) was added, and the mixture was stirred for 1 h. The mixture was diluted with EA (20 mL) and washed with sat. aq. NH₄Cl (20 mL). The organic layer was dried over anhydrous Na₂SO₄ and filtered. The filtrate was concentrated under reduced pressure. The residue was purified by column chromatography (2% MeOH in DCM) to give the crude product. The crude product was purified by TLC (10% MeOH in DCM) to give 30-7 (420 mg, 95.63%) as a white solid.

To a solution of 30-7 (420 mg, 791 μmol) in MeOH (4 mL) was added NH₄F (586 mg, 15.83 mmol) at RT. The mixture was stirred at 90-100° C. for 10 h. The mixture was filtered, and the filtrate was concentrated under reduced pressure. The residue was purified by column chromatography (10% MeOH in DCM) to give the crude product. The crude product was purified by prep-HPLC (neutral condition) to give 30 (201 mg, 61.8% yield, 100% deuterium) as a white solid. ESI-TOF-MS: m/z 303.1 [M+H]⁺, 605.2 [2M+H]⁺.

### Example 14

### Preparation of Compound 31

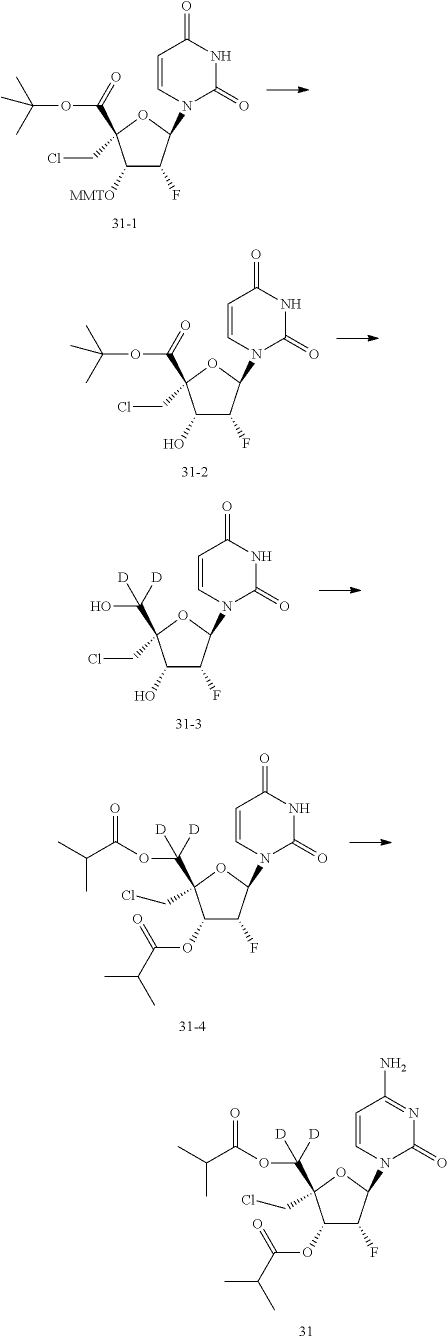

A solution of 31-1 (0.68 g, 1.07 mmol) in AcOH (10 mL) and TFA (0.25 mL) was stirred 1 h at RT. The mixture was evaporated, and the residue coevaporated with MeCN and toluene. Purification on silica column with MeOH:CH₂Cl₂ solvent system (2-12% gradient) afforded 31-1 (0.32 g, 82%).

A mixture of 31-1 (0.32 g, 0.9 mmol) in THF (9 mL) and LiBH₄ (94 mg, 3.6 mmol) was stirred 2 d at RT. The reaction was quenched with AcOH:EtOH, and the mixture evaporated. Purification on silica column with MeOH:CH₂Cl₂ solvent system (4-15% gradient) afforded 31-2 (80 mg, 30%).

A mixture of 31-2 (80 mg, 0.27 mmol) in pyridine (3 mL) and isobutyric anhydride (90 μL, 0.55 mmol) was stirred overnight at RT. The mixture was evaporated, and the residue coevaporated with toluene. Purification on silica column with EtOAc:hexanes solvent system (30-100% gradient) yielded 31-3 (72 mg, 61%) as a white solid.

To a solution of 31-3 (72 mg, 0.17 mmol) in MeCN (2 mL) were added triisopropylphenylsulfonyl chloride (102 mg, 0.34 mmol), DMAP (41 mg, 0.34 mmol) and Et₃N (47 μL, 0.34 mmol). The mixture was stirred at RT for 90 mins, and then ammonia was quickly bubbled (<1 min) through. The mixture was stirred for 10 mins. The mixture was diluted with CH₂Cl₂, washed with 0.1 N HCl, sat. aq. NaHCO₃, and brine, and dried with Na₂SO₄. Purification on silica column with MeOH:CH₂Cl₂ solvent system (4-12% gradient) afforded 31 (46 mg, 60%). MS: m/z=434.00 [M-1].

### Example 15

### Preparation of Compound 32

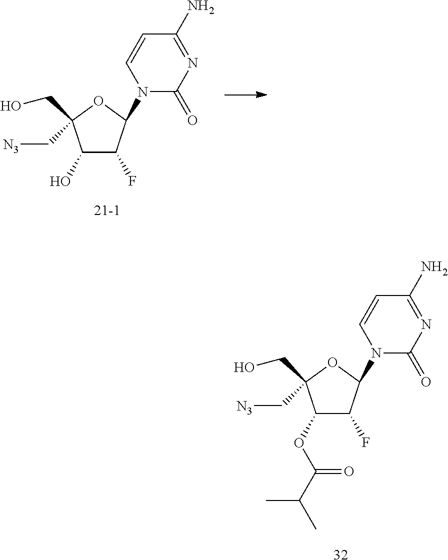

To a solution of isobutiric acid (278 μL, 3 mmol) in THF (5 mL) was added CDI (486 mg, 3 mmol). After 1 h the solution of isobutiric acid imidazolide was added to a stirred solution of 18 (600 mg, 2 mmol), triethylamine (560 μL, 4 mmol) and DMAP (0.2 mmol) in DMF (5 mL). The solution was left overnight at RT. The reaction was partitioned between isopropyl acetate and sat. aq. ammonium chloride. The organic phase was washed with water and concentrated under reduced pressure. 32 (500 mg, 67%) was isolated by column chromatography (10 to 15% MeOH in DCM) followed by crystallization from isopropanol:hexane (1:2) as a white solid. MS: m/z 371 [M+H]⁺.

### Example 16

### Preparation of Compound 33

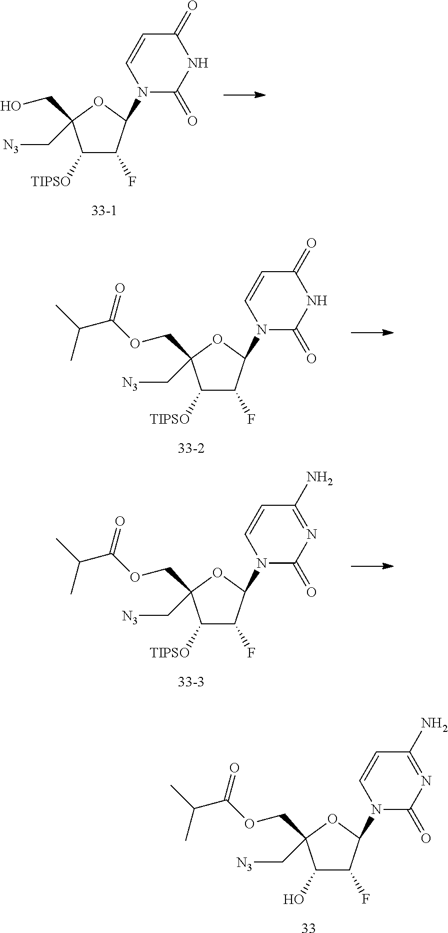

To a stirred solution of 33-1 (2.16 g, 4.73 mmol) in ACN (20 mL) were added triethylamine (1.9 mL, 15 mmol), DMAP (60 mg, 0.5 mmol) and isobutyric anhydride (1.08 mL, 6.5 mmol). The mixture was stirred at RT for 1 h, and then partitioned between isopropyl acetate and sat. aq. sodium bicarbonate solution. The organic phase was separated, washed with water and concentrated. 33-2 (2.1 g, 84%) was isolated by column chromatography using 25 to 50% EA in hexane as a white foam. MS: m/z 528 [M+H]⁺.

33-2 (2.1 g, 3.98 mmol) was dissolved in ACN (15 mL) and the solution was cooled to 0° C. Triethylamine (1.1 mL, 8 mmol) and DMAP (537 mg, 4.4 mmol) were added to the solution followed by addition of triisopropylbenzenesulfonil chloride (1.33 g, 4.4 mmol). The mixture was warmed to RT and then stirred for 1 h. The reaction was quenched with ammonium hydroxide (1 mL). The mixture was stirred for 2 h at RT, diluted with isopropyl acetate and filtered from ammonium salts. The filtrate was washed with water and aq. sodium bicarbonate and then concentrated under reduced pressure. 33-3 (2.1 g, ˜100%) was isolated by column chromatography using 4-10% MeOH in CH₂Cl₂ as a yellowish foam. MS: m/z 527 [M+H]⁺.

33-3 (1.10 g, 2.09 mmol) was dissolved in THF (6 mL). The solution was cooled to 0° C. and treated with 1M TBAF solution in THF (2.1 mL, 2.1 mmol). The reaction was allowed to proceed for 1 h, and then quenched by the addition of a sat. aq. ammonium chloride solution. 33 (450 mg, 58%) was extracted with isopropyl acetate and isolated by column chromatography in 5-15% MeOH in CH₂Cl₂ as an off-white foam, MS: m/z 371 [M+H]⁺.

### Example 184

### Preparation of Compound 34

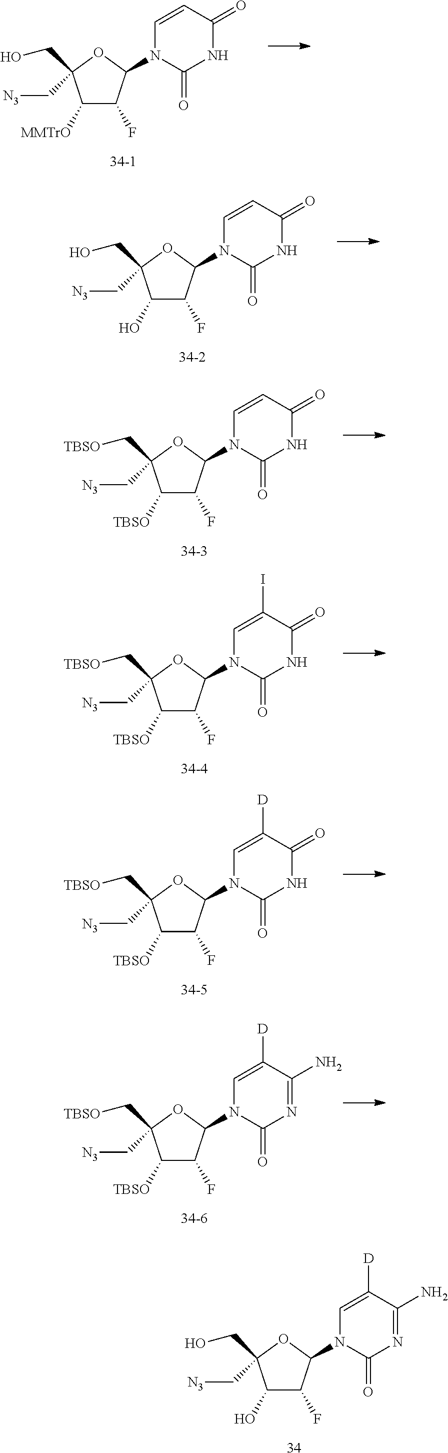

To a solution of 34-1 (1.2 g, 2.09 mmol) in DCE (40 mL) was added TFA (2 mL). The mixture was stirred at RT for 1 h. The mixture was concentrated under reduced pressure, and the residue was purified by column chromatography (3% MeOH in DCM) to give 34-2 (600 mg, 95.3%) as a white solid.

To a solution of 34-2 (600 mg, 1.99 mmol) in pyridine (4 mL) was added imidazole (677 mg, 9.95 mmol) and TBSCl (900 mg, 5.97 mmol) at RT. The mixture was stirred at 60° C. for 16 h, and then concentrated under reduced pressure. The residue was diluted with EA (40 mL) and washed with brine (20 mL). The organic layer was dried over anhydrous MgSO₄ and filtered. The filtrate was concentrated under reduced pressure, and the residue was purified by column chromatography (10% EA in PE) to give 34-3 (700 mg, 65.7%) as a white solid.

To a solution of 34-3 (700 mg, 1.32 mmol) in DCM (52 mL) was added NIS (356 mg, 1.58 mmol) and TFA (1.3 mL). The mixture was stirred at 60° C. for 3 h. After cooling to RT, the solution was extracted with DCM (30 mL), washed with sat. aq. NaHCO₃ and brine (20 mL), dried over anhydrous Na₂SO₄ and filtered. The filtrate was concentrated under reduced pressure. The residue was purified by column chromatography (10% EA in PE) to give 34-4 (400 mg, 46.2%) as a white solid.

A mixture of 34-4 (327 mg, 498 μmol), Bu₃SnH (174 mg, 598 μmol) and 2,2′-azobis(2,4-dimethylvaleronitrile) (25 mg, 100 μmol) in THF-d₈ (10 mL) was stirred at 90-100° C. for 3 h. The mixture was concentrated under reduced pressure. and the residue was purified by column chromatography (10% EA in PE) to give 34-5 (180 mg, 68.00%) as a white solid.

To a solution of 34-5 (210 mg, 395 μmol) in anhydrous MeCN (2 mL) were added DMAP (121 mg, 989 μmol), Et₃N (100 mg, 989 μmol) and 2,4,6-triisopropylbenzene-1-sulfonyl chloride (299 mg, 989 μmol) at RT. The mixture was stirred at RT for 16 h. NH₃.H₂O (1 mL) was added, and the mixture was stirred for 1 h. The mixture was diluted with EA (15 mL) and washed with sat. aq. NH₄Cl (15 mL). The organic layer was dried over anhydrous Na₂SO₄ and filtered. The filtrate was concentrated under reduced pressure, and the residue was purified by column chromatography (2% MeOH in DCM) to give the crude product. The crude product was purified by prep-TLC (10% MeOH in DCM) to give 34-6 (200 mg, 95.42%) as a white solid.

To a solution of 34-6 (200 mg, 0.38 mmol) in MeOH (2 mL) was added NH₄F (210 mg, 5.66 mmol) at RT. The mixture was stirred at 90-100° C. for 16 h. The mixture was filtered, and the filtrate was concentrated under reduced pressure. The residue was purified by column chromatography (10% MeOH in DCM) to give the crude product. The crude product was purified by prep-HPLC (neutral condition) to give 34 (70 mg, 61.8% yield, 78.4% deuterium) as a white solid. ESI-TOF-MS: m/z=302.1 [M+H]⁺, 603.2 [2M+H]⁺.

### Example 18

### Preparation of Compound 35

Dry nucleoside (0.05 mmol) was dissolved in a mixture of PO(OMe)₃ (0.7 mL) and pyridine (0.3 mL). The mixture was evaporated in vacuum for 15 mins at a bath temperature of 42° C., and then cooled down to R.T. N-Methylimidazole (0.009 mL, 0.11 mmol) was added followed by POCl₃ (9 ul, 0.11 mmol), and the mixture was kept at R.T. for 40 mins. The reaction was controlled by LCMS and monitored by the appearance of the corresponding nucleoside 5′-monophosphate. After more than 50% of transformation was achieved, tetrabutylammonium salt of pyrophosphate (150 mg) was added, followed by DMF (0.5 mL) to get a homogeneous solution. After 1.5 hours at ambient temperature, the reaction was diluted with water (10 mL) and loaded on the column HiLoad 16/10 with Q Sepharose High Performance. Separation was done in a linear gradient of NaCl from 0 to 1N in 50 mM TRIS-buffer (pH7.5). Triphosphate was eluted at 75-80% B. Corresponding fractions were concentrated. Desalting was achieved by RP HPLC on Synergy 4 micron Hydro-RP column (Phenominex). A linear gradient of methanol from 0 to 30% in 50 mM triethylammonium acetate buffer (pH 7.5) was used for elution. The corresponding fractions were combined, concentrated and lyophilized 3 times to remove excess of buffer.

| | ³¹P NMR | ³¹P NMR | ³¹P NMR | MS |
|---|---|---|---|---|
| Compound | Pα | Pβ | Pγ | (M − 1) |
| 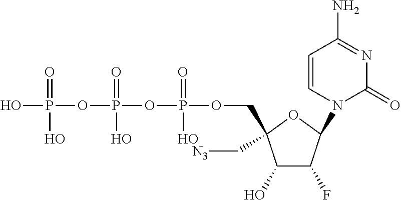 [SMILES: [N-]=[N+]=NC[C@]1(COP(=O)(O)OP(=O)(O)OP(=O)(O)O)O[C@@H](n2ccc(N)nc2=O)[C@H](F)[C@@H]1O] 35 | −5.36(d) | −20.72(t) | −11.40(d) | 539.3 |

### Example 19

### Preparation of Compound 36

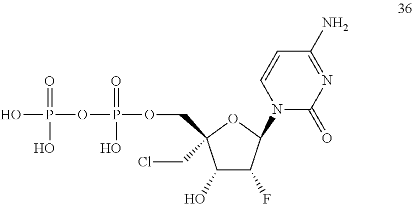 [SMILES: Nc1ccn([C@@H]2O[C@](CCl)(COP(=O)(O)OP(=O)(O)O)[C@@H](O)[C@H]2F)c(=O)n1]

The diphosphate, 36, can be prepared using a similar procedure to preparing the triphosphate of Example 18 with the replacement of tetrabutylammonium salt of pyrophosphate with tetrabutylammonium phosphate (75 mg) and using 0.3 mL of DMF to get the homogeneous solution.

### Example 20

### RSV Assay

The RSV subgenomic replicon 395 HeLa was licensed from Apath (Brooklyn, N.Y.) and was originally developed by Dr. Mark Meeples of Center for Vaccines & Immunity, the Research Institute at Nationwide Children's Hospital in Columbus, Ohio. To generate subgenomic RSV replicon, three glycoprotein genes, those for SH, G, and F, from a full-length recombinant GFP-expressing (rg) RSV antigenomic cDNA were deleted. In their place, a blasticidin S deaminase (bsd) gene was inserted. Through multiple steps, the RSV replicon was established in HeLa cells. The 395 HeLa cells were cultured in Dulbecco's Modified Eagle Medium (DMEM) containing 4500 mg/L D-glucose, L-glutamine, and 110 mg/L sodium pyruvate (Invitrogen, Cat. #11995-040). The medium was further supplemented with 10% (v/v) fetal bovine serum (FBS) (Mediatech, Cat. #35-010-CV), 1% (v/v) penicillin/streptomycin (Mediatech, Cat. #30-002-CI), and 10 μg/mL of Blasticidin (BSD) (Invivogen, Cat. code ant-b1-1). Cells were maintained at 37° C. in a humidified 5% CO₂ atmosphere.

Determination of 50% inhibitory concentration (C₅₀), 90% inhibitory concentration (C₉₀) and 50% cytotoxic concentration (C₅₀) in RSV replicon cells were performed by the following procedure. On the first day, 5000 RSV replicon cells per well were plated in a 96-well plate. On the following day, compounds to be tested were solubilized in 100% DMSO to 100× the desired final testing concentration. Each compound was serially diluted (1:3) up to 9 distinct concentrations. Compounds in 100% DMSO were reduced to 10% (v/v) DMSO by diluting 1:10 in cell culture media. A 10 μL sample of the compounds diluted to 10% (v/v) DMSO with cell culture media was used to treat the RSV replicon cells in 96-well format. The final DMSO concentration was 1% (v/v). Cells were incubated with compounds for 7 days at 37° C. in a 5% CO₂ atmosphere. In each assay, positive control that was previously characterized in the RSV replicon assay was included.

The Renilla Luciferase Assay System (Promega, Cat. #E2820) was used to measure anti-RSV replicon activity. Assay plates were set up as stated above. Luminescence was recorded using a Perkin Elmer multilabel counter Victor3V. EC₅₀, the concentration of the drug required for reducing RSV replicon RNA by 50% in relation to the untreated cell control value, was calculated from the plot of percentage reductions of the optical density (OD) value against the drug concentrations using the Microsoft Excel forecast function.

395 HeLa cell proliferation assay (Promega; CellTiter-Glo Luminescent Cell Viability Assay, Cat. #G7572) was used to measure cell viability. The CellTiter-Glo® Luminescent Cell Viability Assay is a homogeneous method to determine the number of viable cells in culture based on quantitation of the ATP present, which signals the presence of metabolically active cells. Assay plates were set up in the same format as noted above for the replicon assay. CellTiter-Glo reagent (100 μL) was added to each well and incubated at room temperature for 8 minutes. Luminescence was recorded using a Perkin Elmer multilabel counter Victor3V. The CC₅₀, the concentration of the drug required for reducing viable cells by 50% in relation to the untreated cell control value, was calculated from the plot of percentage reductions of the luminescence value against the drug concentrations using the Microsoft Excel forecast function.

Compounds 31 and 34 each had an EC₅₀ value less than 1 μM.

### Example 21

### Combination Studies

RSV with Renilla Reporter

RSV expressing Renilla luciferase (A2-RL-line19F) was generated by Dr. Martin Moore of Emory University, Atlanta, Ga., USA. The in vitro viral kinetics of A2-RL-line19F is similar to that of wild type RSV (See Hotard, A. L., Virology (2012) 434(1):129-136).

Host cell HEp-2 was purchased from ATCC (Cat. #CCL-23) and cells were cultured in DMEM/Ham's F-12 50/50 1× containing L-glutamine and 15 mM HEPES (Mediatech, Cat. #10-092-CM). The medium was further supplemented with 5% (v/v) FBS (Mediatech, Cat. #35-010-CV) and 1% (v/v) penicillin/streptomycin (Mediatech, Cat. #30-002-0). HEp-2 cells were maintained at 37° C. in a humidified 5% CO₂ atmosphere.

### Drug Treatment and Viral Dosing

To determine the effect of a combination of compounds, the following procedure was followed. On the first day, 20,000 HEp-2 cells were plated per well in a 96-well plate. On the following day, test articles were solubilized in 100% DMSO (for chemicals) or 1×PBS (for biologics) to 200× the desired final testing concentration. Subsequently, Compound (A), or a pharmaceutically acceptable salt thereof, was serially diluted (1:3) to 9 distinct concentrations “horizontally” in a 96-well plate, and Compound (B), or a pharmaceutically acceptable salt thereof, was serially diluted (1:3) to 7 distinct concentrations “vertically” in 96-well plate. The serially diluted 200× test articles were then diluted 1:10 into cell culture media to generate 20× test articles. A 5 μL aliquot of the 20× test articles was added in a checkerboard fashion to the cells with 90 μL existing media. Space was also allotted for titrations of each of the compounds alone to be used as reference controls. After 12 hour pre-incubation of test articles, A2-RL-line19F at an MOI of 0.5 was added to the plate and further incubated for 2 days at 37° C. in a 5% CO₂.

### Determination of Anti-RSV Activity

The Renilla Luciferase Assay System (Promega, Cat. # E2820) was used to measure anti-RSV replicon activity. Assay plates were set up as stated above. Luminescence was recorded using a Perkin Elmer multilabel counter Victor3V.

### Cell Viability Assay

Promega CellTiter-Glo Luminescent Cell Viability Assay, Cat. #G7572) was used to measure cell viability. The CellTiter-Glo® Luminescent Cell Viability Assay is a homogeneous method to determine the number of viable cells in culture based on quantitation of the adenosine triphosphate (ATP) present, which signals the presence of metabolically active cells. Assay plates were set up in the same format the anti-RSV assay, except that no virus was added to the cell viability assay. A 100-μL aliquot of CellTiter-Glo reagent was added to each well and incubated at room temperature for 8 minutes. Luminescence was recorded using a Perkin Elmer multilabel counter Victor3V.

### Data Analysis

Each experiment was performed at N=5 for both anti-RSV activity and cell viability. Mean percent inhibition of the replicon values from the 5 experiments was generated and for anti-RSV activity, it was analyzed using two drug interaction analysis models, Isobologram Analysis and/or Prichard's Model.

### Isobologram Analysis

The effects of drug-drug combinations were evaluated by the Loewe additivity model in which the experimental data were analyzed using CalcuSyn (Biosoft, Ferguson, Mo.), a computer program based on the method of Chou and Talalay. The combination index (CI) value and the isobologram for each experimental combination were calculated. CI values of <1,1, and >1 indicate synergy, additive effect, and antagonism, respectively. Under the synergy category, CI<0.1 is considered very strong synergism; CI 0.1-0.3 strong synergism; CI 0.3-0.7 synergism and CI 0.7-0.85 moderate synergism. The isobologram analysis, which graphically represents additive, synergistic, and antagonistic drug effects, was also used to model the interaction of antiviral activities. In this representation, an effective concentration (C) value of one drug is plotted on one axis and corresponding EC value of a second drug is plotted on the second axis; the line connecting these two points represents the amount of each drug in a combination that would be required to reach the equivalent EC value, given that their effects are additive.

### Prichard's Model (MacSynergy II)

MacSynergy II software was kindly provided by Dr. M. Prichard (University of Michigan). This program allows the three-dimensional examination of drug interactions of all data points generated from the checkerboard combination of two inhibitors with Bliss-Independence model. Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB=faA+faB−faA·faB with faA and faB representing the fraction of possible responses, e.g. % inhibition, of compounds A and B at amounts dA and dB, respectively, and describes the % inhibition of a combination of compounds A and B at amount (dA+dB). If faAB>faA+faB−faA·faB then we have Bliss synergy; if faAB<faA+faB−faA·faB then we have Bliss antagonism. The 95% synergy/antagonism volumes are the summation of the differences between the observed inhibition and the 95% confidence limit on the prediction of faAB under the Bliss independence model. Table 1 shows the volumes and corresponding volume descriptions for the results of the Bliss Independence Analysis. MacSynergy II was used for data analysis.

The synergy volume results for the combinations are provided in Table 2.

TABLE 1
MacSynergy II Volume Descriptions

| Volume (μM² %) | Volume Description |
|---|---|
| <25 | Additive |
| 25-50 | Minor synergism |
| 50-100 | Significant synergism |
| >100 | Strong synergism |

TABLE 2

| | | Class of | Synergy | |
|---|---|---|---|---|
| Compound | | Compound | Volume | Observed |
| (A) | Compound (B) | (B) | (μM² %) | Result |
| 1 | Palivizumab | Anti-RSV | 115 | strongly |
| | | Antibody | | synergistic |
| 1 | BMS-433771 | Fusion | 320 | strongly |
| | | Inhibitor | | synergistic |
| 1 | 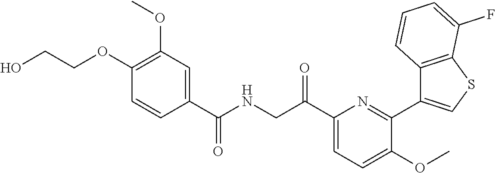 [SMILES: COc1cc(C(=O)NCC(=O)c2ccc(OC)c(-c3csc4c(F)cccc34)n2)ccc1OCCO] | Polymerase Inhibitor | 12.2 | Additive |
| 1 | Ribavirin | IMPDH | 4.7 | Additive |
| | | Inhibitor | | |
| 1 | interferon alfacon-1 | interferon | 81 | Significant |
| | | | | synergism |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A method for ameliorating or treating a paramyxovirus virus infection comprising administering to a subject infected with the paramyxovirus virus an effective amount of a combination of Compound (A) and one or more of Compound (B), or a pharmaceutical acceptable salt of any of the foregoing, wherein:
the Compound (A) has the structure: 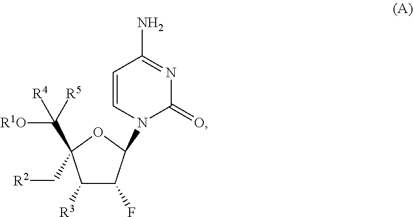
wherein:
R¹ is selected from the group consisting of H, an optionally substituted acyl, an optionally substituted O-linked amino acid and 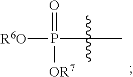
R² is chloro or azido;
R³ is selected from the group consisting of OH, —OC(═O)R^{A1} and an optionally substituted O-linked amino acid;
R⁴ and R⁵ are independently H or D;
R⁶ and R⁷ is independently absent, H, 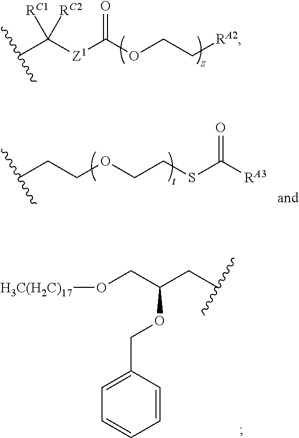
R^{A1} is an optionally substituted C₁₋₂₄ alkyl;
R^{A2} is independently selected from the group consisting of H, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl, 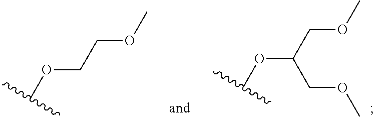
R^{A3} is selected from the group consisting of H, an optionally substituted C₁₋₂₄ alkyl and an optionally substituted aryl;
R^{C1} and R^{C2} are independently selected from the group consisting of H, an optionally substituted C₁₋₂₄ alkyl and an optionally substituted aryl;
s is 0, 1, 2 or 3;
t is 0 or 1; and
Z¹ is O or S;
one or more of Compound (B) is selected from the group consisting of an anti-RSV antibody, a fusion protein inhibitor, an N-protein inhibitor, a RSV polymerase inhibitor, an IMPDH inhibitor, an interferon and an other compound that inhibits the RSV virus, or a pharmaceutically acceptable salt of any of the foregoing; and
the paramyxovirus virus infection is selected from the group consisting of a respiratory syncytial virus infection, a parainfluenza virus infection and a metapneumovirus infection.

2. A method for ameliorating or treating a paramyxovirus virus infection comprising contacting a cell infected with the paramyxovirus virus with an effective amount of a combination of Compound (A) and one or more of Compound (B), or a pharmaceutical acceptable salt of any of the foregoing, wherein:
the Compound (A) has the structure: 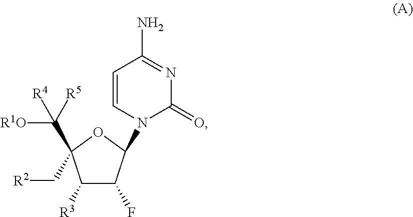
wherein:
R¹ is selected from the group consisting of H, an optionally substituted acyl, an optionally substituted O-linked amino acid, 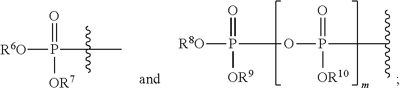
R² is chloro or azido;
R³ is selected from the group consisting of OH, —OC(═O)R^{A1} and an optionally substituted O-linked amino acid;
R⁴ and R⁵ are independently H or D;
R⁶ and R⁷ is independently absent, H, 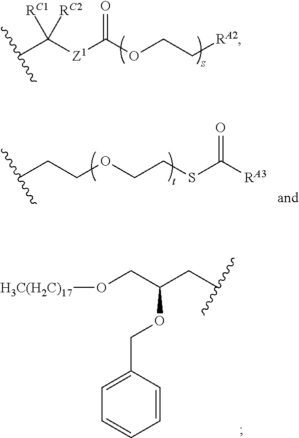
R⁸, R⁹ and each R¹⁰ are independently absent or H;
R^{A1} is an optionally substituted C₁₋₂₄ alkyl;
R^{A2} is independently selected from the group consisting of H, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl, 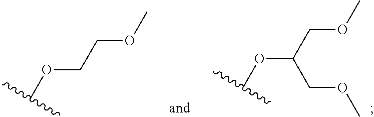
R^{A3} is selected from the group consisting of H, an optionally substituted C₁₋₂₄ alkyl and an optionally substituted aryl;
R^{C1} and R^{C2} are independently selected from the group consisting of H, an optionally substituted C₁₋₂₄ alkyl and an optionally substituted aryl;
m is 1 or 2;
s is 0, 1, 2 or 3;
t is 0 or 1; and
Z¹ is O or S;
one or more of Compound (B) is selected from the group consisting of an anti-RSV antibody, a fusion protein inhibitor, an N-protein inhibitor, a RSV polymerase inhibitor, an IMPDH inhibitor, an interferon and an other compound that inhibits the RSV virus, or a pharmaceutically acceptable salt of any of the foregoing; and
the paramyxovirus virus infection is selected from the group consisting of a respiratory syncytial virus infection, a parainfluenza virus infection and a metapneumovirus infection.

3. (canceled)

4. The method of claim 2, wherein the paramyxovirus virus infection is a respiratory syncytial virus infection.

5. (canceled)

6. (canceled)

7. The method of claim 2, wherein the paramyxovirus virus infection is a parainfluenza virus infection.

8. The method of claim 2, wherein the paramyxovirus virus infection is a metapneumovirus infection.

9. The method of claim 2, wherein one or more of Compound (B) is an anti-RSV antibody.

10. The method of claim 9, wherein the anti-RSV antibody is selected from the group consisting of
RSV-IGIV (RespiGam®)
palivizumab (Synagis®, a chimeric humanized IgG monoclonal antibody) and
motavizumab (MEDI-524, humanized monoclonal antibody).

11. The method of claim 2, wherein one or more of Compound (B) is a fusion protein inhibitor.

12. The method of claim 11, wherein the fusion protein inhibitor is selected from the group consisting of
1-cyclopropyl-3-[[1-(4-hydroxybutyl)benzimidazol-2-yl]methyl]imidazo[4,5-c]pyridin-2-one (BMS-433771),
4,4″-bis-{4,6-bis-[3-(bis-carbamoylmethyl-sulfamoyl)-phenylamino]-(1,3,5)triazin-2-ylamino}-biphenyl-2,2″-disulfonic-acid (RFI-641),
4,4′-Bis[4,6-di[3-aminophenyl-N,N-bis(2-carbamoylethyl)-sulfonilimino]-1,3,5-triazine-2-ylamino]-biphenyl-2,2′-disulfonic acid, disodium salt (CL387626),
2-[[2-[[1-(2-aminoethyl)-4-piperidinyl]amino]-4-methyl-1H-benzimidazol-1-yl]-6-methyl-3-pyridinol (JNJ-2408068),
2-[[6-[[[2-(3-Hydroxypropyl)-5-methylphenyl]amino]methyl]-2-[[3-(morpholin-4-yl)propyl]amino]benzimidazol-1-yl]methyl]-6-methylpyridin-3-ol (TMC-353121),
5,5′-bis[1-(((5-amino-1H-tetrazolyl)imino)methyl)]2,2′,4″-methylidynetrisphenol (VP-14637, MDT-637),
N-(2-hydroxyethyl)-4-methoxy-N-methyl-3-(6-methyl-[1,2,4]triazolo[3,4-a]phthalazin-3-yl)benzene sulfonamide (P13),
2-((2-((1-(2-aminoethyl)piperidin-4-yl)amino)-4-methyl-1H-benzo[d]imidazol-1-yl)methyl)-6-methylpyridin-3-ol (R170591),
1,4-bis(3-methylpyridin-4-yl)-1,4-diazepane (C15),
(R)-9b-(4-chlorophenyl)-1-(4-fluorobenzoyl)-2,3-dihydro-1H-imidazo[1′,2′:1,2]pyrrolo[3,4-c]pyridin-5(9bH)-one (BTA9981),
[2,2-bis(docosyloxy-oxymethyl)propyl-5-acetaoamido-3,5-dideoxy-4,7,8,9-tetra-O-(sodium-oxysulfonyl)-D-glycero-D-galacto-2-nonulopyranosid]onate (MBX-300), BTA-C286,
N-(2-((S)-2-(5-((S)-3-aminopyrrolidin-1-yl)-6-methylpyrazolo[1,5-a]pyrimidin-2-yl)piperidine-1-carbonyl)-4-chlorophenyl)methanesulfonamide (GS-5806),
an anti-RSV nanobody and
a peptide fusion inhibitor selected from the group consisting of a peptide having the sequence DEFDASISQVNEKINQSLAFIRKSDELL (T-67), and a peptide having the sequence FDASISQVNEKINQSLAFIRKSDELLHNVNAGKST (T-118),
or a pharmaceutically acceptable salt of any of the foregoing.

13. (canceled)

14. The method of claim 2, wherein one or more of Compound (B) is an N-protein inhibitor.

15. The method of claim 14, wherein the N-protein inhibitor is selected from the group consisting of (S)-1-(2-fluorophenyl)-3-(2-oxo-5-phenyl-2,3-dihydro-1H-benzo[e][1,4]diazepin-3-yl)urea (RSV-604), STP-92 (siRNA delivered through nanoparticle based delivery systems, Sirnaomics) and iKT-041 (Inhibikase), or a pharmaceutically acceptable salt thereof.

16. The method of claim 2, wherein one or more of Compound (B) is a RSV polymerase inhibitor.

17. The method of claim 16, wherein the RSV polymerase inhibitor is selected from the group consisting of
6-{4-[(biphenyl-2-ylcarbonyl)amino]benzoyl}-N-cyclopropyl-5,6-dihydro-4H-thieno[3,2-d][1]benzazepine-2-carboxamide (YM-53403),
N-cyclopropyl-5-(4-(2-(pyrrolidin-1-yl)benzamido)benzoyl)-5,6,7,10-tetrahydrobenzo[b]cyclopenta[d]azepine-9-carboxamide,
6-(4-(2-(2-oxa-7-azaspiro[3.5]nonan-7-yl)nicotinamido)benzoyl)-N-cyclopropyl-5,6-dihydro-4H-benzo[b]thieno[2,3-d]azepine-2-carboxamide,
4-amino-8-(3-{[2-(3,4-dimethoxyphenyl)ethyl]amino}propyl)-6,6-dimethyl-2-(4-methyl-3-nitrophenyl)-1H-imidazo[4,5-h]isoquinoline-7,9(6H,8H)-dione and
6-(4-(2-(2-oxa-7-azaspiro[3.5]nonan-7-yl)nicotinamido)benzoyl)-N-cyclopropyl-5,6-dihydro-4H-benzo[b]thieno[2,3-d]azepine-2-carboxamide (AZ27),
or a pharmaceutically acceptable salt of any of the foregoing.

18. The method of claim 2, wherein one or more of Compound (B) is selected from the group consisting of an IMPDH inhibitor, an interferon and an other compound that inhibits the RSV virus.

19. The method of claim 18, wherein the IMPDH inhibitor is selected from the group consisting of
ribavirin,
5-ethynyl-1-beta-D-ribofuranosylimidazole-4-carboxamide (EICAR),
4-hydroxy-3-beta-D-ribofuranosylpyrazole-5-carboxamide (pyrazofurin),
1-((2R,3R,4S,5R)-3,4-dihydroxy-5-(hydroxymethyl)tetrahydrofuran-2-yl)-1H-1,2,4-triazole-3-carboximidamide (Taribavirin, viramidine),
1,3,4-thiadiazol-2-ylcyanamide (LY253963),
tetrahydrofuran-3-yl-3-(3-(3-methoxy-4-(oxazol-5-yl)phenyl)ureido)benzylcarbamate (VX-497),
(4E)-6-(4-Hydroxy-6-methoxy-7-methyl-3-oxo-1,3-dihydro-2-benzofuran-5-yl)-4-methylhex-4-enoic acid (Mycophenolic acid) and
2-morpholin-4-ylethyl-(E)-6-(4-hydroxy-6-methoxy-7-methyl-3-oxo-1H-2-benzofuran-5-yl)-4-methylhex-4-enoate (Mycophenolate Mofetil),
or a pharmaceutically acceptable salt of any of the foregoing.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 18, wherein the other compound is selected from the group consisting of
a double stranded RNA oligonucleotide,
5-methyl-N-[4-(trifluoromethyl)phenyl]-isoxazole-4-carboxamide (leflumomide),
N-(2-chloro-4-methylphenyl)-2-((1-(4-methoxyphenyl)-1H-benzo[d]imidazol-2-yl)thio)propanamide (JMN3-003),
Medi-559,
Medi-534,
Medi-557,
an intratracheal formulation of recombinant human CC10 (CG-100),
high titer, human immunoglobulin (RI-001, ADMA Biologics Inc.) and
a non-neutralizing mAb against the G protein (mAb 131-2G),
or a pharmaceutically acceptable salt of any of the foregoing.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The of claim 2, wherein R² is chloro.

58. The method of claim 2, wherein R² is azido.

59. The method or of claim 2, wherein R³ is OH or R³ is —OC(═O)R^{A1}.

60. (canceled)

61. (canceled)

62. (canceled)

63. (canceled)

64. (canceled)

65. The method of claim 2, wherein R¹ is hydrogen.

66. The method of claim 2, wherein R¹ is an optionally substituted acyl.

67. (canceled)

68. (canceled)

69. (canceled)

70. (canceled)

71. (canceled)

72. The method of claim 2, wherein R¹ is 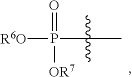
wherein R⁶ and R⁷ are independently absent or H.

73. (canceled)

74. (canceled)

75. (canceled)

76. (canceled)

77. (canceled)

78. (canceled)

79. The method of claim 2, wherein R¹ is 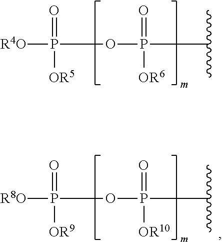
wherein m is 1, and R⁸, R⁹ and each R¹⁰ are independently absent or H.

80. (canceled)

81. The method of claim 2, wherein R¹ is 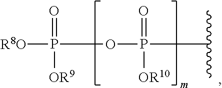
wherein m is 2, and R⁸, R⁹ and each R¹⁰ are independently absent or H.

82. The method of claim 2, wherein Compound (A) is selected from the group consisting of: 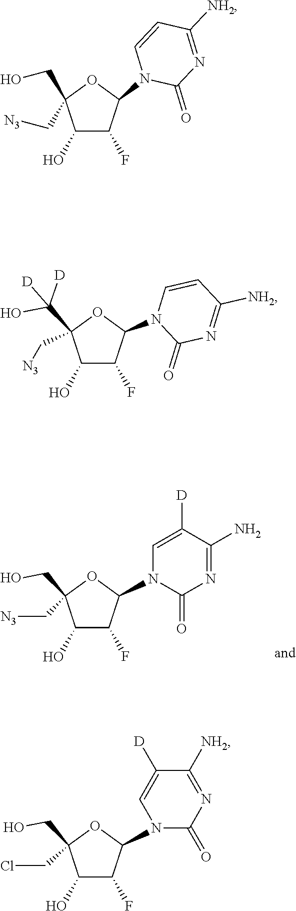 [SMILES: [N-]=[N+]=NC[C@]1(CO)O[C@@H](n2ccc(N)nc2=O)[C@H](F)[C@@H]1O ; [2H]C([2H])(O)[C@@]1(CN=[N+]=[N-])O[C@@H](n2ccc(N)nc2=O)[C@H](F)[C@@H]1O ; [2H]c1cn([C@@H]2O[C@@](CO)(CN=[N+]=[N-])[C@@H](O)[C@H]2F)c(=O)nc1N ; [2H]c1cn([C@@H]2O[C@@](CO)(CCl)[C@@H](O)[C@H]2F)c(=O)nc1N]
or a pharmaceutically acceptable salt of any of the foregoing.

83. The method of claim 2, wherein Compound (A) is selected from the group consisting of: 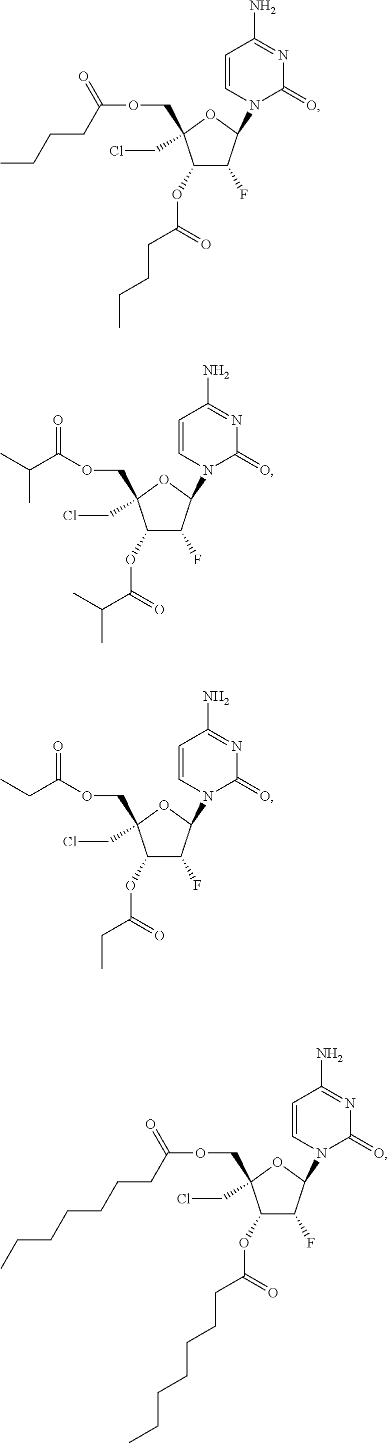 [SMILES: CCCCC(=O)OC[C@@]1(CCl)O[C@@H](n2ccc(N)nc2=O)[C@H](F)[C@@H]1OC(=O)CCCC ; CC(C)C(=O)OC[C@@]1(CCl)O[C@@H](n2ccc(N)nc2=O)[C@H](F)[C@@H]1OC(=O)C(C)C ; CCC(=O)OC[C@@]1(CCl)O[C@@H](n2ccc(N)nc2=O)[C@H](F)[C@@H]1OC(=O)CC ; CCCCCCCC(=O)OC[C@@]1(CCl)O[C@@H](n2ccc(N)nc2=O)[C@H](F)[C@@H]1OC(=O)CCCCCCC] 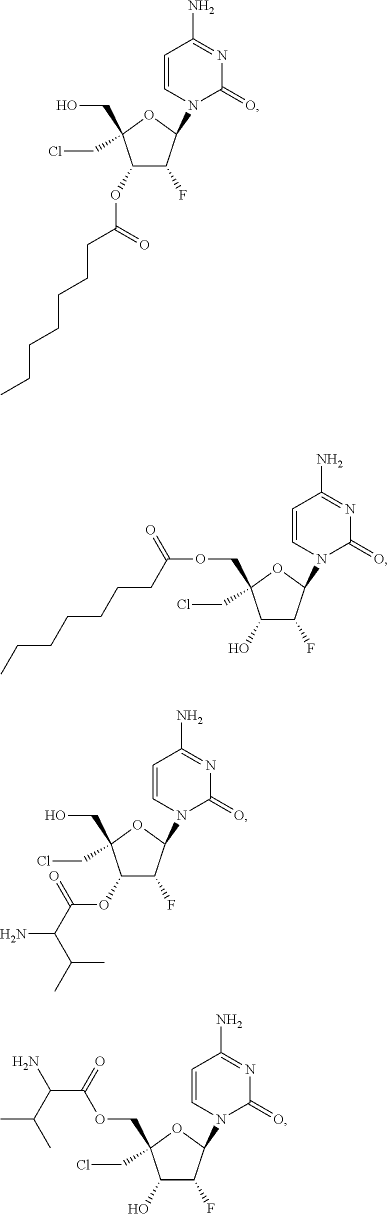 [SMILES: CCCCCCCC(=O)O[C@H]1[C@@H](F)[C@H](n2ccc(N)nc2=O)O[C@@]1(CO)CCl ; CCCCCCCC(=O)OC[C@@]1(CCl)O[C@@H](n2ccc(N)nc2=O)[C@H](F)[C@@H]1O ; CC(C)C(N)C(=O)O[C@H]1[C@@H](F)[C@H](n2ccc(N)nc2=O)O[C@@]1(CO)CCl ; CC(C)C(N)C(=O)OC[C@@]1(CCl)O[C@@H](n2ccc(N)nc2=O)[C@H](F)[C@@H]1O] 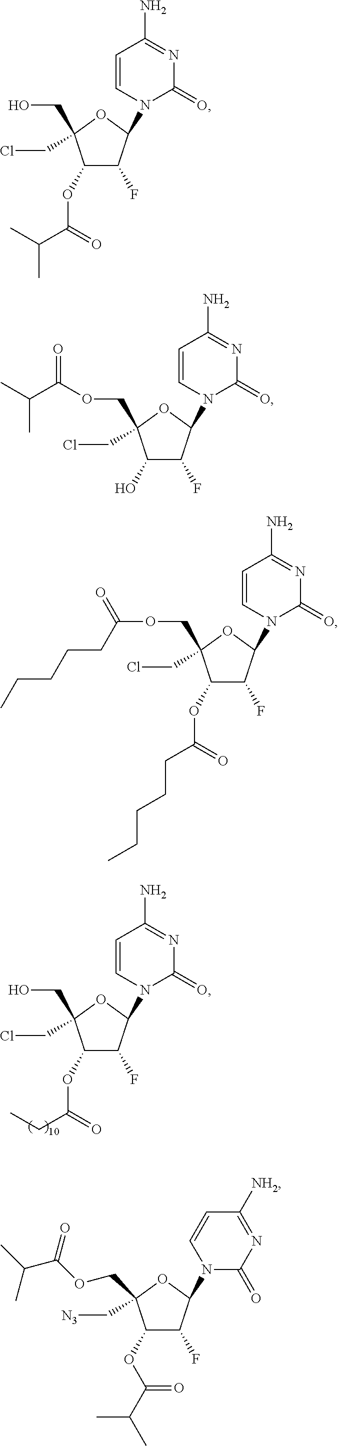 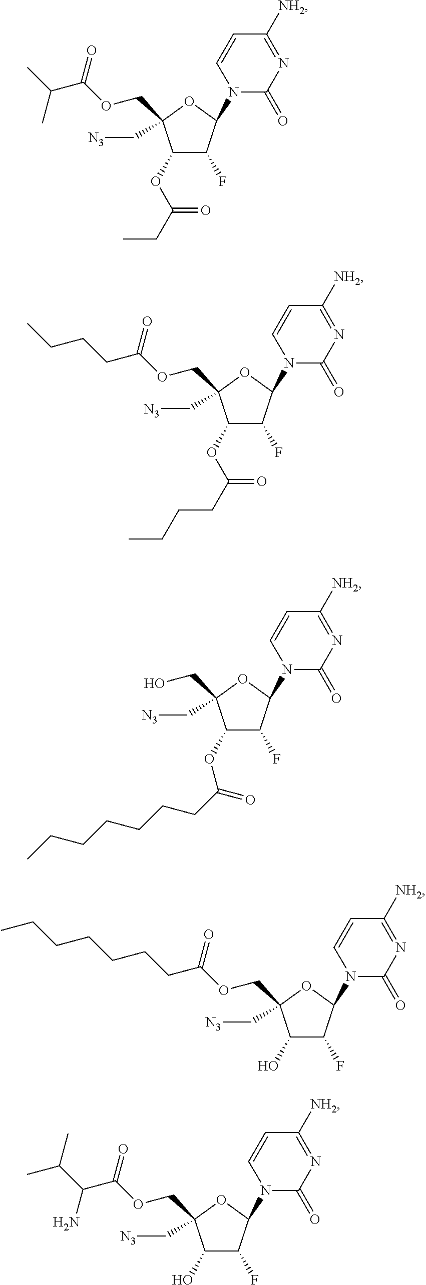 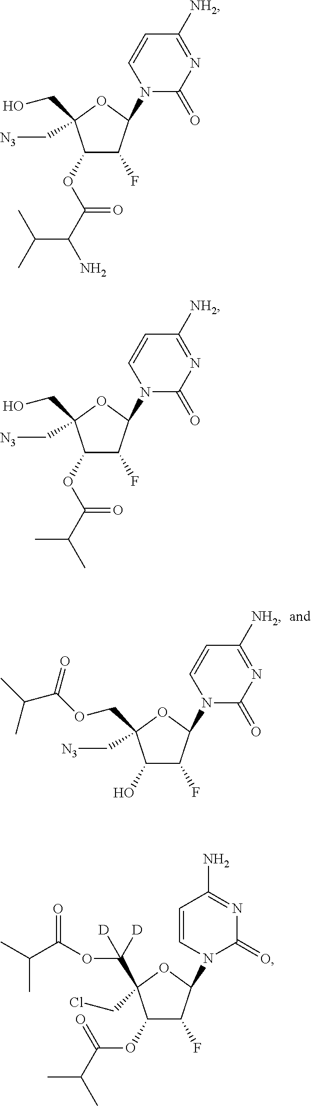 [SMILES: CC(C)C(N)C(=O)O[C@H]1[C@@H](F)[C@H](n2ccc(N)nc2=O)O[C@@]1(CO)CN=[N+]=[N-] ; CC(C)C(=O)O[C@H]1[C@@H](F)[C@H](n2ccc(N)nc2=O)O[C@@]1(CO)CN=[N+]=[N-] ; CC(C)C(=O)OC[C@@]1(CN=[N+]=[N-])O[C@@H](n2ccc(N)nc2=O)[C@H](F)[C@@H]1O ; [2H]C([2H])(OC(=O)C(C)C)[C@@]1(CCl)O[C@@H](n2ccc(N)nc2=O)[C@H](F)[C@@H]1OC(=O)C(C)C]
or a pharmaceutically acceptable salt of any of the foregoing.

84. The method of claim 2, wherein Compound (A) is selected from the group consisting of: 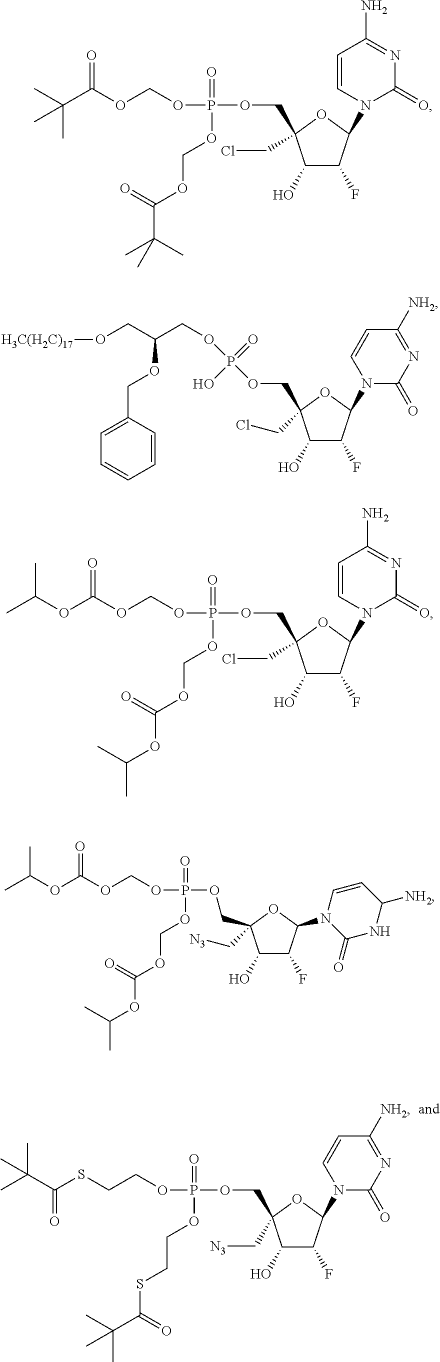 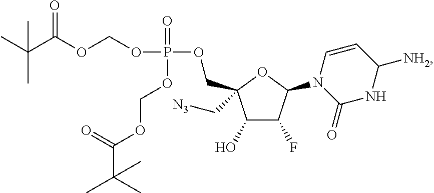 [SMILES: CC(C)(C)C(=O)OCOP(=O)(OCOC(=O)C(C)(C)C)OC[C@@]1(CN=[N+]=[N-])O[C@@H](N2C=CC(N)NC2=O)[C@H](F)[C@@H]1O]
or a pharmaceutically acceptable salt of any of the foregoing.

85. The method of claim 2, wherein Compound (A) is selected from the group consisting of: 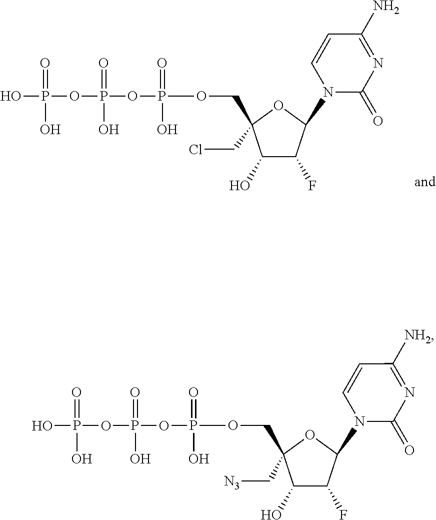 [SMILES: Nc1ccn([C@@H]2O[C@](CCl)(COP(=O)(O)OP(=O)(O)OP(=O)(O)O)[C@@H](O)[C@H]2F)c(=O)n1 ; [N-]=[N+]=NC[C@]1(COP(=O)(O)OP(=O)(O)OP(=O)(O)O)O[C@@H](n2ccc(N)nc2=O)[C@H](F)[C@@H]1O]
or a pharmaceutically acceptable salt of any of the foregoing.


SEQUENCE LISTING (4 sequences)

SEQ ID NO: 1 - protein, 28 aa, Artificial Sequence
  Core polypeptide
DEFDASISQVNEKINQSLAFIRKSDELL

SEQ ID NO: 2 - protein, 35 aa, Artificial Sequence
  Core polypeptide
FDASISQVNEKINQSLAFIRKSDELLHNVNAGKST

SEQ ID NO: 3 - DNA, 21 bp, Artificial Sequence
  Combined DNA/RNA Molecule Synthetic oligonucleotide
ggcucuuagcaaagucaagtt

SEQ ID NO: 4 - DNA, 21 bp, Artificial Sequence
  Combined DNA/RNA Molecule Synthetic oligonucleotide
cuugacuuugcuaagagcctt